(19) United States
(12) Patent Application Publication
(10) Pub. No.: US 2022/0218732 A1
(43) Pub. Date: Jul. 14, 2022

(54) Selective Inhibitors Of Protein Arginine Methyltransferase 5 (PRMT5)

(71) Applicant: Prelude Therapeutics, Incorporated, Wilimington, DE (US)

(72) Inventors: Juan Luengo, Phoenixville, PA (US); Raul A. Leal, Newark, DE (US); Hong Lin, Exton, PA (US); Rupa Shetty, Blue Bell, PA (US); Krishna Vaddi, Chadds Ford, PA (US)

(21) Appl. No.: 17/601,471

(22) PCT Filed: Apr. 3, 2020
(86) PCT No.: PCT/US2020/026650

§ 371 Date: Oct. 5, 2021

Related U.S. Application Data
(60) Provisional application No. 62/830,077, filed on Apr. 5, 2019.

(51) Int. Cl. A61K 31/7064; A61P 37/00

(52) U.S. Cl. CPC A61K 31/7064; A61P 37/00

(57) ABSTRACT

The disclosure is directed to methods of treating disease using compounds of Formula I, Formula II, Formula III, Formula IV, Formula V, or Formula VI:

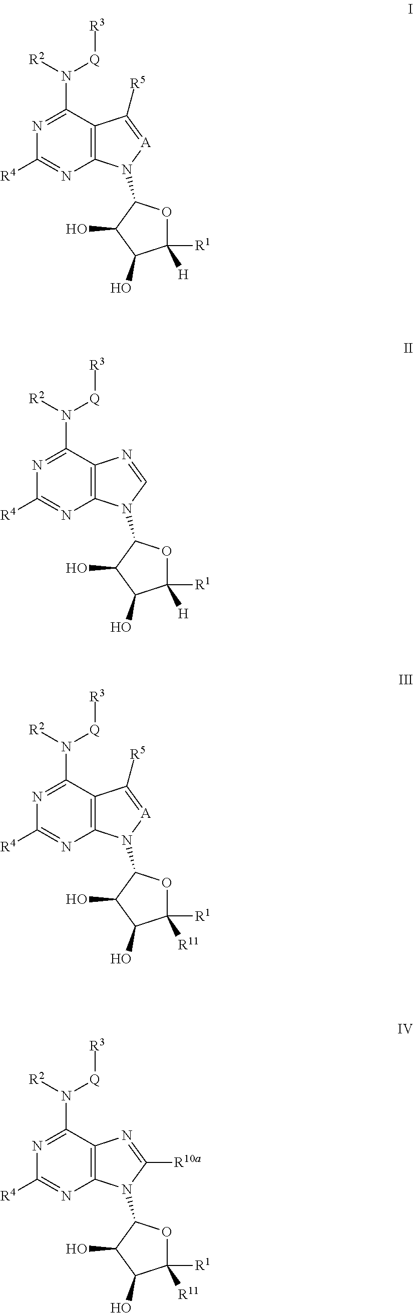

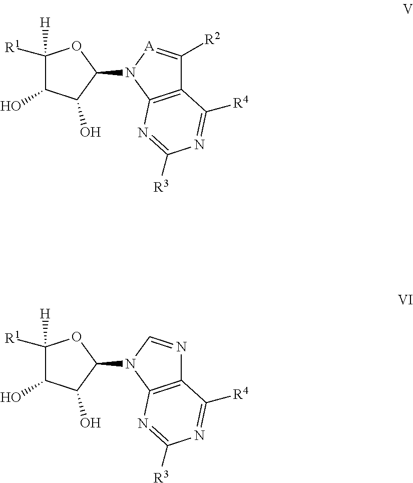

### CROSS-REFERENCE TO RELATED APPLICATION

The application claims priority to U.S. Provisional Patent Application No. 62/830,077, filed on Apr. 5, 2019, the entirety of which is incorporated by reference herein.

### TECHNICAL FIELD

The disclosure is directed to PRMT5 inhibitors and methods of their use.

### BACKGROUND

Protein arginine methylation is a common post-translational modification that regulates numerous cellular processes, including gene transcription, mRNA splicing, DNA repair, protein cellular localization, cell fate determination, and signaling. Three types of methyl-arginine species exist: ω NG monomethylarginine (MMA), ω NG, NG asymmetric dimethylarginine (ADMA) and ω NG, N′G symmetric dimethylarginine (SDMA). The formation of methylated arginines is catalyzed by the protein arginine methyl transferases (PRMTs) family of methyltransferases. Currently, there are nine PRMTs annotated in the human genome The majority of these enzymes are Type I enzymes (PRMT1, -2, -3, -4, -6, -8) that are capable of mono- and asymmetric dimethylation of arginine, with S-adenosylmethionine (SAM) as the methyl donor. PRMT-5, -7 and -9 are considered to be Type II enzymes that catalyze symmetric dimethylation of arginines. Each PRMT species harbors the characteristic motifs of seven beta strand methyltransferases (Katz et al., 2003), as well as additional “double E” and “THW” sequence motifs particular to the PRMT subfamily.

PRMT5 is as a general transcriptional repressor that functions with numerous transcription factors and repressor complexes, including BRG1 and hBRM, Blimp1, and Snail. This enzyme, once recruited to a promoter, symmetrically dimethylates H3R8 and H4R3. Importantly, the H4R3 site is a major target for PRMT1 methylation (ADMA) and is generally regarded as a transcriptional activating mark. Thus, both H4R3me2s (repressive; me2s indicates SDMA modification) and H4R3me2a (active; me2a indicates ADMA modification) marks are produced in vivo. The specificity of PRMT5 for H3R8 and H4R3 can be altered by its interaction with COPR5 and this could perhaps play an important role in determining PRMT5 corepressor status.

PRMTs have been implicated in a number of diseases and disorders. Compounds that inhibit PRMTs, including PRMT5, are needed.

### SUMMARY

The disclosure is directed to methods of treating diseases or disorders, including rejection of transplanted organs or tissue; graft-versus-host diseases brought about by transplantation; autoimmune syndromes, multiple sclerosis, myasthenia gravis; pollen allergies; type I diabetes; prevention of psoriasis; Crohn's disease; ulcerative colitis, acute respiratory distress syndrome; adult respiratory distress syndrome; influenza; COVID-19 (coronavirus disease); or post-infectious autoimmune diseases including rheumatic fever and post-infectious glomerulonephritis in a patient by administering to the patient an effective amount of a compounds of Formula I or Formula II.

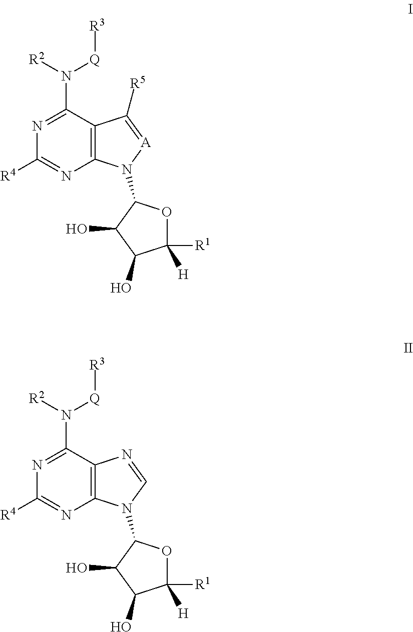

or a pharmaceutically acceptable salt or solvate thereof;
wherein
A is CH or N;
Q is NH, NR⁶ or O;
R¹ is —C₀-C₆alk-C₃-C₆cycloalkyl, —C₀-C₆alk-C₃-C₆halocycloalkyl; —C₂-C₆alkenyl, —C₂-C₆haloalkenyl, —C₀-C₆alk-C₁-C₆alkyl, —C₀-C₆alk-C₁-C₆haloalkyl, —C₀-C₆alk-C≡CH, —C₀-C₆alk-C≡C—C₁-C₆alkyl, —C₀-C₆alk-C≡C—C₁-C₆haloalkyl, —C₀-C₆alk-C≡C—C₃-C₆cycloalkyl, —C₁-C₆alk-aryl, —C₁-C₆alk-S—C₁-C₆alkyl, —C₁-C₆alk-S—C₁-C₆haloalkyl, —C₁-C₆alk-S—C₃-C₆cycloalkyl; —C₁-C₆alk-S—C₃-C₆halocycloalkyl; —C₁-C₆alk-O—C₁-C₆alkyl, —C₁-C₆alk-O—C₃-C₆cycloalkyl, —C₁-C₆alk-S—CH₂-aryl, or —C₁-C₆alk-C(O)NH-aryl, —C₀-C₆alk-heteroaryl, —C₁-C₆alk-O-heteroaryl, —C₁-C₆alk-S-heteroaryl, or —C₁-C₆alk-NH-heteroaryl;
R² is H, —C₁-C₆alkyl, —C₁-C₆haloalkyl, or —C₀-C₆alk-C₃-C₆cycloalkyl;
R³ is H, —C₁-C₆alkyl, —C₁-C₆haloalkyl, —C₀-C₆alk-C₃-C₆cycloalkyl, —C(O)R⁷, —C(O)OR⁷, or —C(O)NR^{8a}R^{8b};
R⁴ is H, halo, —C₁-C₆alkyl, or NH₂;
R⁵ is H, halo, CN, —C₁-C₆alkyl, —C₂-C₄alkenyl, —C₂-C₄haloalkenyl, C₂-C₄cyanoalkenyl, —C₀-C₆alk-C≡CH, —C₀-C₆alk-C≡C—C₁-C₆alkyl, —C₁-C₄haloalkyl, —C₂-C₆heterocycloalkyl, oxo-substituted-C₂-C₆heterocycloalkyl, —C₃-C₆cycloalkyl, —C₀-C₃-alk-C(O)R⁹, —CR⁸R^{8′}CN, —CH₂NR⁸R^{8′}, —C₀-C₆alk-OH, —NR⁸R^{8′}, —N(R⁹)CN, —O—C₁-C₄alkyl, —NR⁹CONR⁸R^{8′}, —OCONR⁸R^{8′} or —NR⁹C(O)OR^{9a};
R⁶ is —C₁-C₆alkyl or C₀-C₆alk-C₃-C₆cycloalkyl;
R⁷ is H, C₁-C₆alkyl, or C₀-C₆alk-C₃-C₆cycloalkyl;
R^{8a} and R^{8b} are each independently H, C₁-C₆alkyl, or —C₀-C₆alk-OC₁-C₆alkyl, or R^{8a} and R^{8b}, together with the atom to which they are attached, form a C₂-C₆heterocycloalkyl ring;
R⁸ and R^{8′} are each independently H, C₁-C₆alkyl, or —C₀-C₆alk-OC₁-C₆alkyl;
or R⁸ and R^{8′} together with the atom to which they are attached, form a C₃-C₆cycloalkyl ring or a C₂-C₆heterocycloalkyl ring;
R⁹ is H, —C₁-C₆alkyl, or —C₀-C₆alk-C₃-C₆cycloalkyl; and
R^{9a} is —C₁-C₆alkyl, or —C₀-C₆alk-C₃-C₆cycloalkyl.

Stereoisomers of the compounds of Formula I or Formula II, and the pharmaceutical salts and solvates thereof, are also described. Methods of using compounds of Formula I or Formula II are described, as well as pharmaceutical compositions including the compounds of Formula I or Formula II.

The disclosure is also directed to methods of treating diseases or disorders, including rejection of transplanted organs or tissue; graft-versus-host diseases brought about by transplantation; autoimmune syndromes, multiple sclerosis, myasthenia gravis; pollen allergies; type I diabetes; prevention of psoriasis; Crohn's disease; ulcerative colitis, acute respiratory distress syndrome; adult respiratory distress syndrome; influenza; COVID-19 (coronavirus disease); or post-infectious autoimmune diseases including rheumatic fever and post-infectious glomerulonephritis in a patient by administering to the patient an effective amount of a compounds of Formula III or IV:

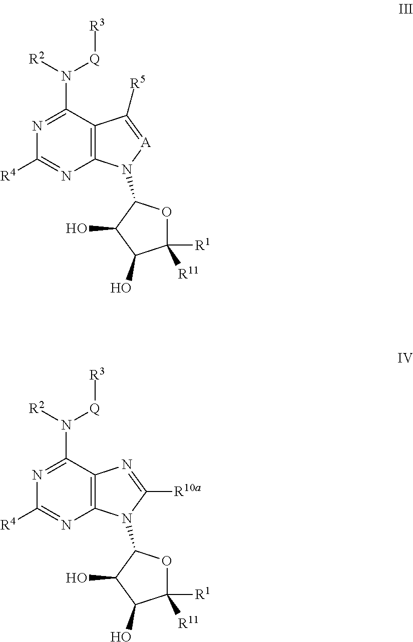

or a pharmaceutically acceptable salt or solvate thereof;

wherein
A is CH, CR¹⁰, or N;
Q is NH, NR⁶, or O;
R¹ is —C₀-C₆alk-C₃-C₆cycloalkyl, —C₀-C₆alk-C₃-C₆halocycloalkyl; —C₂-C₆alkenyl, —C₂-C₆haloalkenyl, —C₀-C₆alk-C₁-C₆alkyl, —C₀-C₆alk-C₁-C₆haloalkyl, —C₀-C₆alk-C≡CH, —C₀-C₆alk-C≡C—C₁-C₆alkyl, —C₀-C₆alk-C≡C—C₁-C₆haloalkyl, —C₀-C₆alk-C≡C—C₃-C₆cycloalkyl, —C₁-C₆alk-aryl, —C₁-C₆alk-S—C₁-C₆alkyl, —C₁-C₆alk-S—C₁-C₆haloalkyl, —C₁-C₆alk-S—C₃-C₆cycloalkyl; —C₁-C₆alk-S—C₃-C₆halocycloalkyl, —C₁-C₆alk-O—C₁-C₆alkyl, —C₁-C₆alk-O—C₃-C₆cycloalkyl, —C₁-C₆alk-S—CH₂-aryl, —C₁-C₆alk-C(O)NH-aryl, —C₀-C₆alk-S-aryl, —C₀-C₆alk-S(O)aryl, —C₀-C₆alk-S(O)₂aryl, —C₀-C₆alk-Oaryl, —C₀-C₆alk-heteroaryl, —C₁-C₆alk-O-heteroaryl, —C₁-C₆alk-S-heteroaryl, or —C₁-C₆alk-NH-heteroaryl;
R² is H, —C₁-C₆alkyl, —C₁-C₆haloalkyl, or —C₀-C₆alk-C₃-C₆cycloalkyl;
R³ is H, —C₁-C₆alkyl, —C₁-C₆haloalkyl, —C₀-C₆alk-C₃-C₆cycloalkyl, —C(O)R⁷, —C(O)OR⁷, or —C(O)NR^{8a}R^{8b};
R⁴ is H, halo, —C₁-C₆alkyl, or NH₂;
R⁵ is H, halo, CN, —C₁-C₆alkyl, —C₂-C₄alkenyl, —C₂-C₄haloalkenyl, C₂-C₄cyanoalkenyl, —C₀-C₆alk-C≡CH, —C₀-C₆alk-C≡C—C₁-C₆alkyl, —C₁-C₄haloalkyl, —C₂-C₆heterocycloalkyl, oxo-substituted-C₂-C₆heterocycloalkyl, —C₃-C₆cycloalkyl, —C₀-C₃alk-C(O)R⁹, —CR⁸R^{8′}CN, —CH₂NR⁸R^{8′}, —C₀-C₆alk-OH, —NR⁸R^{8′}, —N(R⁹)CN, —O—C₁-C₄alkyl, —NR⁹CONR⁸R^{8′}, —OCONR⁸R^{8′}, or —NR⁹C(O)OR^{9a};
R⁶ is C₁-C₆alkyl, or C₀-C₆alk-C₃-C₆cycloalkyl
R⁷ is H, C₁-C₆alkyl, or C₀-C₆alk-C₃-C₆cycloalkyl;
R^{8a} and R^{8b} are each independently H, C₁-C₆alkyl, or —C₀-C₆alk-OC₁-C₆alkyl, or R^{8a} and R^{8b}, together with the atom to which they are attached, form a C₂-C₆heterocycloalkyl ring;
R⁸ and R^{8′} are each independently H, C₁-C₆alkyl, or —C₀-C₆alk-OC₁-C₆alkyl;
or R⁸ and R^{8′}, together with the atom to which they are attached, form a C₃-C₆cycloalkyl ring or a C₂-C₆heterocycloalky ring;
R⁹ is H, —C₁-C₆alkyl, or C₀-C₆alk-C₃-C₆cycloalkyl;
R^{9a} is —C₁-C₆alkyl, or C₀-C₆alk-C₃-C₆cycloalkyl;
R¹⁰ is halo or —C₁-C₆alkyl;
R^{10a} is H, halo or —C₁-C₆alkyl;
R¹¹ is H, —C₁-C₆alkyl, —C₁-C₆haloalkyl, —C₀-C₆alk-C₃-C₆cycloalkyl, —C₀-C₆alk-C₃-C₆halocycloalkyl, —C₀-C₆alk-OH, —C₀-C₆alk-NH₂, —C₀-C₆alk-NH—C₁-C₆alkyl, —C₀-C₆alk-N(C₁-C₆alkyl)-C₁-C₆alkyl, —C₀-C₆alk-NH—C₃-C₆cycloalkyl, or —C₀-C₆alk-N(C₁-C₆alkyl)-C₃-C₆cycloalkyl;
or R¹¹ and R¹, together with the atom to which they are attached, form a C₃-C₆cycloalkyl ring or a heterocycloalkyl ring.

Stereoisomers of the compounds of Formula III or Formula IV, and the pharmaceutical salts and solvates thereof, are also described. Methods of using compounds of Formula III or Formula IV are described, as well as pharmaceutical compositions including the compounds of Formula III or Formula IV.

The disclosure is also directed to methods of treating diseases or disorders, including rejection of transplanted organs or tissue; graft-versus-host diseases brought about by transplantation; autoimmune syndromes, multiple sclerosis, myasthenia gravis; pollen allergies; type I diabetes; prevention of psoriasis; Crohn's disease; ulcerative colitis, acute respiratory distress syndrome; adult respiratory distress syndrome; influenza; COVID-19 (coronavirus disease); or post-infectious autoimmune diseases including rheumatic fever and post-infectious glomerulonephritis in a patient by administering to the patient an effective amount of a compounds of Formula V or Formula VI:

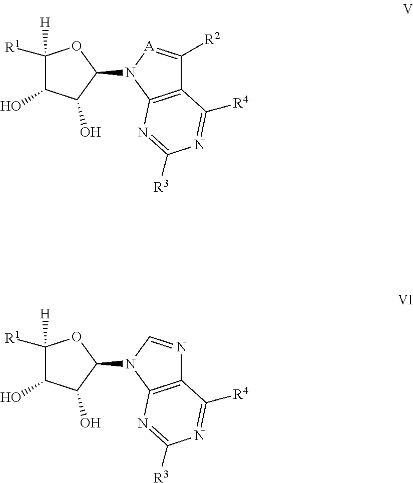

or a pharmaceutically acceptable salt or solvate thereof;

wherein
A is CH or N;
R¹ is —C₁-C₆alk-aryl, —C₁-C₆alk-heteroaryl, —C₁-C₆alk-C≡CH, —C₁-C₆alk-C≡C—C₁-C₆alkyl, —C₁-C₆alk-C≡C—C₁-C₆haloalkyl, or —C₁-C₆alk-C≡C—C₃-C₆cycloalkyl;
R² is H, or halo;
R³ is H, halo, NH₂, or C₁-C₆alkyl; and
R⁴ is NH₂ or CH₃.

Stereoisomers of the compounds of Formula V or Formula VI, and the pharmaceutical salts and solvates thereof, are also described. Methods of using compounds of Formula V or Formula VI, are described, as well as pharmaceutical compositions including the compounds of Formula V or Formula VI.

### BRIEF DESCRIPTION OF THE DRAWINGS

FIG. 1 is an ORTEP representation of Example 69.

FIG. 2 is an XRPD scan of Example 92A.

FIG. 3 is a Differential Scanning Calorimetry (“DSC”) profile of Example 92A.

FIG. 4 is a thermogravimetric analysis (“TGA”) scan of Example 92A.

FIG. 5 is a Dynamic Vapor Sorption (“DVS”) profile of Example 92A.

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any subcombination.

The term “alkyl,” when used alone or as part of a substituent group, refers to a straight- or branched-chain hydrocarbon group having from 1 to 12 carbon atoms (“C₁-C₁₂”), preferably 1 to 6 carbons atoms (“C₁-C₆”), in the group. Examples of alkyl groups include methyl (Me, C₁alkyl), ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), isohexyl (C₆alkyl), and the like.

The term “halo” when used alone or as part of a substituent group refers to chloro, fluoro, bromo, or iodo.

The term “haloalkyl” when used alone or as part of a substituent group refers to refers to an alkyl group wherein one or more of the hydrogen atoms has been replaced with one or more halogen atoms. Halogen atoms include chlorine, fluorine, bromine, and iodine. Examples of haloalkyl groups of the disclosure include, for example, trifluoromethyl (—CF₃), chloromethyl (—CH₂Cl), and the like.

The term “cycloalkyl” when used alone or as part of a substituent group refers to cyclic-containing, non-aromatic hydrocarbon groups having from 3 to 10 carbon atoms (“C₃-C₁₀”), preferably from 3 to 6 carbon atoms (“C₃-C₆”). Examples of cycloalkyl groups include, for example, cyclopropyl (C₃), cyclobutyl (C₄), cyclopropylmethyl (C₄), cyclopentyl (C₅), cyclohexyl (C₆), 1-methylcyclopropyl (C₄), 2-methylcyclopentyl (C₄), adamantanyl (C₁₀), and the like.

The term “halocycloalkyl” when used alone or as part of a substituent group refers to a cycloalkyl group wherein one or more of the hydrogen atoms has been replaced with one or more halogen atoms. Halogen atoms include chlorine, fluorine, bromine, and iodine. Examples of cycloalkyl groups include, for example, chlorocyclopropyl (C₃), fluorocyclobutyl (C₄), bromocyclopentyl (C₅), iodocyclohexyl (C₆), and the like.

The term “heterocycloalkyl” when used alone or as part of a substituent group refers to any three to ten membered monocyclic or bicyclic, saturated ring structure containing at least one heteroatom selected from the group consisting of O, N and S. Where N is a heteroatom in the heterocycloalkyl group, the N may be substituted with H, —C₁-C₃alkyl, —C₁-C₃haloalkyl, or C₃-C₆cycloalkyl. The heterocycloalkyl group may be attached at any heteroatom or carbon atom of the ring such that the result is a stable structure. Examples of suitable heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahydrofuranyl, tetrahydropyranyl, piperazinyl, and the like.

The term “oxo-substituted-heterocycloalkyl” when used alone or as part of a substituent group refers to a heterocycloalkyl group wherein at least one of the carbon atoms in the ring is substituted with an oxo group. Examples of oxo-substituted heterocycloalkyl groups include, but are not limited to, 2-aziridinonyl, 2-azetidinonyl, pyrrolidinonyl, dioxolanonyl, imidazolidinonyl, pyrazolidinonyl, piperazinonyl, piperidinonyl, dioxanonyl, dithianonyl, thiomorpholinonyl, oxazepanonyl, oxiranonyl, oxetanonyl, quinuclidinonyl, tetrahydrofuranonyl, tetrahydropyranonyl, piperazinonyl, and the like.

The term “alkenyl” when used alone or as part of a substituent group refers to a straight- or branched-chain group having from 2 to 12 carbon atoms (“C₂-C₁₂”), preferably 2 to 4 carbons atoms (“C₂-C₄”), in the group, wherein the group includes at least one carbon-carbon double bond. Examples of alkenyl groups include vinyl (—CH═CH₂; C₂alkenyl) allyl (—CH₂—CH═CH₂; C₃alkenyl), propenyl (—CH═CHCH₃; C₃alkenyl); isopropenyl (—C(CH₃)═CH₂; C₃alkenyl), butenyl (—CH═CHCH₂CH₃; C₄alkenyl), sec-butenyl (—C(CH₃)═CHCH₃; C₄alkenyl), iso-butenyl (—CH═C(CH₃)₂; C₄alkenyl), 2-butenyl (—CH₂CH═CHCH₃; C₄alkyl), pentenyl (—CH═CHCH₂CH₂CH₃; C₅alkenyl), and the like.

The term “haloalkenyl” when used alone or as part of a substituent group refers to an alkenyl group wherein at least one carbon atom in the group is substituted by one or more halogen atoms. Halogen atoms include chlorine, fluorine, bromine, and iodine.

The term “cyanoalkenyl” when used alone or as part of a substituent group refers to an alkenyl group wherein at least one carbon atom in the group is substituted by one or more cyano groups.

The term “cycloalkenyl,” when used alone or as part of a substituent group refers to cyclic, non-aromatic hydrocarbon groups having from 3 to 10 carbon atoms (“C₃-C₁₀”), preferably from 3 to 6 carbon atoms (“C₃-C₆”) and containing at least one carbon-carbon double bond. For example, cycloalkenyl groups include, but are not limited to cyclopropenyl, cyclobutenyl, and the like.

The term “aryl” when used alone or as part of a substituent group refers to a mono- or bicyclic-aromatic hydrocarbon ring structure having 6 to 10 carbon atoms in the ring, wherein one or more of the carbon atoms in the ring is optionally substituted with a halogen (halo) atom, a —C₁-C₃ alkyl group, an amino-substituted —C₁-C₃alkyl group, a —C₁-C₃haloalkyl group, an amino group (i.e., NH₂), or a substituted amino group. Amino-substituted —C₁-C₃ alkyl groups include —CH₂—NH₂, —CH₂CH₂—NH₂, and the like. C₁-C₃haloalkyl groups include, for example, —CF₃, —CH₂CF₃, and the like. Substituted amino groups include, for example, —NH—C(O)—NH₂. Halogen atoms include chlorine, fluorine, bromine, and iodine. Examples of aryl groups (substituted and unsubstituted) include phenyl, naphtyl, fluorophenyl, difluorophenyl, chlorophenyl, dichlorophenyl, bromophenyl, iodophenyl, chlorofluorophenyl, fluoronaphthyl, difluoronaphthyl, chloronaphthyl, bromonaphthyl, iodonaphthyl, methylphenyl, ethylphenyl, (trifluoromethyl)phenyl, methyl-trifluoromethylphenyl, fluoro-trifluoromethylphenyl and the like. The term “aryl” also includes a mono- or bicyclic-aromatic hydrocarbon ring structure having 6 or 10 carbon atoms in the ring, wherein two adjacent carbon atoms in the ring are optionally substituted such that said two adjacent carbon atoms and their respective substituents form a heterocyclic ring. Thus, aryl groups include, for example, 2,3-dihydrobenzofuran and 1,3-benzodioxole.

The term “heteroaryl” when used alone or as part of a substituent group refers to a mono- or bicyclic-aromatic ring structure including carbon atoms as well as up to four heteroatoms selected from nitrogen, oxygen, and sulfur. Heteroaryl rings can include a total of 5, 6, 9, or 10 ring atoms. The heteroaryl moiety can be unsubstituted or one or more of the carbon atoms in the ring can be substituted with a halogen atom; an amino group; a substituted amino group, including an amino group substituted with a —C₁-C₆ cycloalkyl group or a —C₁-C₆ alkyl group; —C₁-C₃ alkyl group, or a —C₁-C₃ haloalkyl group. Halogen atoms include chlorine, fluorine, bromine, and iodine. Examples of heteroaryl groups include but are not limited to, pyrrolyl, furyl, thiophenyl (thienyl), 5-chlorothiophen-2-yl, oxazolyl, imidazolyl, purazolyl, isoxazolyl, isothiazolyl, triazolyl, thiadiazolyl, pyrazolyl, pyridyl, pyridazinyl, pyrimidinyl, pyrazinyl, pyranyl, furazanyl, indolizinyl, indolyl, isoindolinyl, indazolyl, benzofuranyl, benzothiophenyl, benzimidazolyl, benzthiazolyl, purinyl, quinolizinyl, quinolinyl, 2-amino-3-bromoquinolin-7-yl, 2-amino-3-chloroquinolin-7-yl, 2-((cyclopropylmethyl)amino)quinolin-7-yl, 2-(methylamino)quinolin-7-yl, 2-aminoquinolin-7-yl, isoquinolinyl, isothiazolyl, cinnolinyl, phthalazinyl, quinazolinyl, quinoxalinyl, naphthyridinyl, pteridinyl, and the like.

When a range of carbon atoms is used herein, for example, C₁-C₆, all ranges, as well as individual numbers of carbon atoms are encompassed. For example, “C₁-C₃” includes C₁-C₃, C₁-C₂, C₂-C₃, C₁, C₂, and C₃.

The term “C₁-C₆alk” when used alone or as part of a substituent group refers to an aliphatic linker having 1, 2, 3, 4, 5, or 6 carbon atoms and includes, for example, —CH₂—, —CH(CH₃)—, —CH(CH₃)—CH₂—, and —C(CH₃)₂—. The term “—C₀alk-” refers to a bond. In some aspects, the C₁-C₆alk can be substituted with one or more —OH, —O—C₁-C₆alkyl (e.g., —OCH₃), —NH₂, or halo (e.g., —F, —Cl, —Br, with —F being preferred) substituents. Thus, C₁-C₆alk encompasses, for example, —CH(Me)-, —CH(OH)—, —CH(CH₂OH)—, —CH(Me)(OH)—, —CH(NH₂)—, —CH(Me)(NH₂)—, —CH(F)—, —CH(Me)(F)—, and the like. C¹alk groups, for example, include:

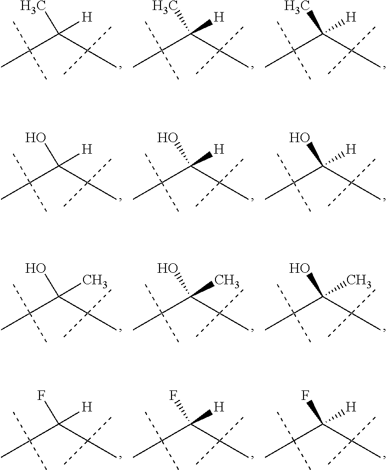

and the like.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I or Formula II with one or more solvent molecules.

“Subject” includes mammals, for example, humans. The terms “human,” “patient,” and “subject” are used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder. In some embodiments, “treating” or “treatment” refers to prophylactic treatment, i.e, preventing the onset of the disease or disorder.

“Compounds of the present disclosure,” and equivalent expressions, are meant to embrace compounds of Formula I, Formula II, Formula III, Formula IV, Formula V, and/or Formula VI as described herein, as well as their subgenera, which expression includes the stereoisomers of compounds of Formula I, Formula II, Formula III, Formula IV, Formula V, and/or Formula VI, as well as the pharmaceutically acceptable salts and solvates, where the context so permits.

As used herein, the term “isotopic variant” refers to a compound that contains proportions of isotopes at one or more of the atoms that constitute such compound that is greater than natural abundance. For example, an “isotopic variant” of a compound can be radiolabeled, that is, contain one or more radioactive isotopes, or can be labeled with non-radioactive isotopes such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. Where a chiral center exists in a structure, but no specific stereochemistry is shown for that center, both enantiomers, individually or as a mixture of enantiomers, are encompassed by that structure. The methods for the determination of stereochemistry and the separation of stereoisomers are well-known in the art.

The disclosure is directed to compounds of Formula I or Formula II. In some aspects, the disclosure is directed to compounds of Formula I.

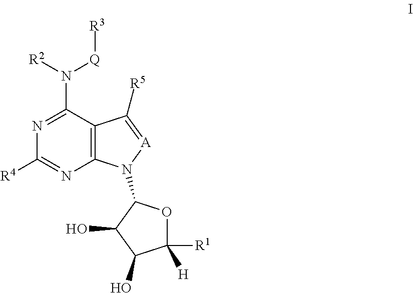

In other aspects, the disclosure is directed to compounds of Formula II:

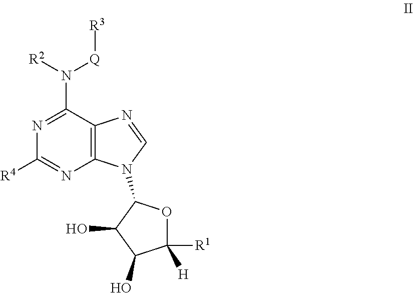

According to the disclosure, A in Formula I is N or CH. In some aspects, A is N and the compounds of Formula I are of Formula IA:

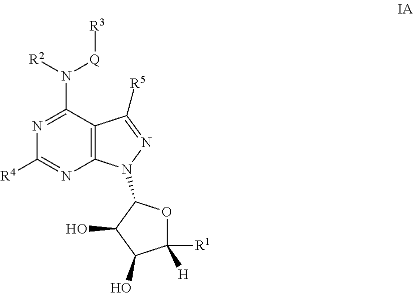

In other aspects, A is CH and the compounds of Formula I are of Formula IB:

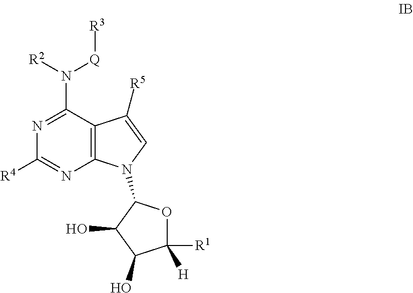

According to the disclosure, Q in Formula I or Formula II is NH, NR⁶ or O. In some embodiments of Formula I or II, Q is NH. In other embodiments, Q is O. In yet other embodiments, Q is NR⁶.

According to the disclosure, R¹ in Formula I or Formula II is —C₀-C₆alk-C₃-C₆cycloalkyl, —C₀-C₆alk-C₃-C₆halocycloalkyl; —C₂-C₆alkenyl, —C₂-C₆haloalkenyl, —C₀-C₆alk-C₁-C₆alkyl, —C₀-C₆alk-C₁-C₆haloalkyl, —C₀-C₆alk-C≡CH, —C₀-C₆alk-C≡C—C₁-C₆alkyl, —C₀-C₆alk-C≡C—C₁-C₆haloalkyl, —C₀-C₆alk-C≡C—C₃-C₆cycloalkyl, —C₁-C₆alk-aryl, —C₁-C₆alk-S—C₁-C₆alkyl, —C₁-C₆alk-S—C₁-C₆haloalkyl, —C₁-C₆alk-S—C₃-C₆cycloalkyl; —C₁-C₆alk-S—C₃-C₆halocycloalkyl; —C₁-C₆alk-O—C₁-C₆alkyl, —C₁-C₆alk-O—C₃-C₆cycloalkyl, —C₁-C₆alk-S—CH₂-aryl, —C₁-C₆alk-C(O)NH-aryl, —C₀-C₆alk-heteroaryl, —C₁-C₆alk-O-heteroaryl, —C₁-C₆alk-S-heteroaryl, or —C₁-C₆alk-NH-heteroaryl.

In some aspects, R¹ in Formula I or Formula II is —C₀-C₆alk-C₁-C₆alkyl, —C₀-C₆alk-C₁-C₆haloalkyl, —C₀-C₆alk-C≡CH, —C₀-C₆alk-C≡C—C₁-C₆alkyl, —C₀-C₆alk-C≡C—C₁-C₆haloalkyl, —C₀-C₆alk-C≡C—C₃-C₆cycloalkyl, or —C₁-C₆alk-aryl.

In other aspects, R¹ in Formula I or Formula II is —C₀-C₆alk-C₃-C₆cycloalkyl, for example, —C₀alk-C₃cycloalkyl, —C₁alk-C₃cycloalkyl, —C₂alk-C₃cycloalkyl, —C₃alk-C₃cycloalkyl, —C₄alk-C₃cycloalkyl, —C₅alk-C₃cycloalkyl, —C₆alk-C₃cycloalkyl, —C₀alk-C₄cycloalkyl, —C₁alk-C₄cycloalkyl, —C₂alk-C₄cycloalkyl, —C₃alk-C₄cycloalkyl, —C₄alk-C₄cycloalkyl, —C₅alk-C₄cycloalkyl, —C₆alk-C₄cycloalkyl, —C₀alk-C₅cycloalkyl, —C₁alk-C₅cycloalkyl, —C₂alk-C₅cycloalkyl, —C₃alk-C₅cycloalkyl, —C₄alk-C₅cycloalkyl, —C₅alk-C₅cycloalkyl, —C₆alk-C₅cycloalkyl, —C₀alk-C₆cycloalkyl, —C₁alk-C₆cycloalkyl, —C₂alk-C₆cycloalkyl, —C₃alk-C₆cycloalkyl, —C₄alk-C₆cycloalkyl, —C₅alk-C₆cycloalkyl, or —C₆alk-C₆cycloalkyl. Thus, in some aspects, R¹ is —CH₂-cyclopropyl.

In some aspects, R¹ in Formula I or Formula II is —C₀-C₆alk-C₃-C₆halocycloalkyl, for example, —C₀alk-C₃halocycloalkyl, —C₁alk-C₃halocycloalkyl, —C₂alk-C₃halocycloalkyl, —C₃alk-C₃halocycloalkyl, —C₄alk-C₃halocycloalkyl, —C₅alk-C₃halocycloalkyl, —C₆alk-C₃halocycloalkyl, —C₀alk-C₄halocycloalkyl, —C₁alk-C₄halocycloalkyl, —C₂alk-C₄halocycloalkyl, —C₃alk-C₄halocycloalkyl, —C₄alk-C₄halocycloalkyl, —C₅alk-C₄halocycloalkyl, —C₆alk-C₄halocycloalkyl, —C₀alk-C₅halocycloalkyl, —C₁alk-C₅halocycloalkyl, —C₂alk-C₅halocycloalkyl, —C₃alk-C₅halocycloalkyl, —C₄alk-C₅halocycloalkyl, —C₅alk-C₅halocycloalkyl, —C₆alk-C₅halocycloalkyl, —C₀alk-C₆halocycloalkyl, —C₁alk-C₆halocycloalkyl, —C₂alk-C₆halocycloalkyl, —C₃alk-C₆halocycloalkyl, —C₄alk-C₆halocycloalkyl, —C₅alk-C₆halocycloalkyl, or —C₆alk-C₆halocycloalkyl.

In some aspects, R¹ in Formula I or Formula II is —C₂-C₆alkenyl, for example, vinyl, allyl, and the like.

In some aspects, R¹ in Formula I or Formula II is —C₂-C₆haloalkenyl, for example, —C(F)═CHMe, —C(F)═CH₂, and the like.

In some aspects, R¹ in Formula I or Formula II is —C₀-C₆alk-C₁-C₆alkyl, for example, —C₀alk-C₁alkyl, —C₁alk-C₁alkyl, —C₂alk-C₁alkyl, —C₃alk-C₁alkyl, —C₄alk-C₁alkyl, —C₄alk-C₁alkyl, —C₆alk-C₁alkyl, —C₀alk-C₂alkyl, —C₁alk-C₂alkyl, —C₂alk-C₂alkyl, —C₃alk-C₂alkyl, —C₄alk-C₂alkyl, —C₅alk-C₂alkyl, —C₆alk-C₂alkyl, —C₀alk-C₃alkyl, —C₁alk-C₃alkyl, —C₂alk-C₃alkyl, —C₃alk-C₃alkyl, —C₄alk-C₃alkyl, —C₅alk-C₃alkyl, —C₆alk-C₃alkyl, —C₀alk-C₄alkyl, —C₁alk-C₄alkyl, —C₂alk-C₄alkyl, —C₃alk-C₄alkyl, —C₄alk-C₄alkyl, —C₅alk-C₄alkyl, —C₆alk-C₄alkyl, —C₀alk-C₅alkyl, —C₁alk-C₅alkyl, —C₂alk-C₅alkyl, —C₃alk-C₅alkyl, —C₄alk-C₅alkyl, —C₅alk-C₅alkyl, —C₆alk-C₅alkyl, —C₀alk-C₆alkyl, —C₁alk-C₆alkyl, —C₂alk-C₆alkyl, —C₃alk-C₆alkyl, —C₄alk-C₆alkyl, —C₅alk-C₆alkyl, —C₀alk-C₆alkyl, methyl, ethyl, propyl, isopropyl, butyl, isobutyl, s-butyl, t-butyl, pentyl, —CH(OH)—C₁-C₆alkyl, —CH(F)—C₁-C₆alkyl, —CH(NH₂)—C₁-C₆alkyl, —CH(Me)-C₁-C₆alkyl, —C(Me)(OH)—C₁-C₆alkyl, and the like.

In other aspects, R¹ in Formula I or Formula II is —C₀-C₆alk-C₁-C₆haloalkyl, for example, —C₀alk-C₁haloalkyl, —C₁alk-C₁haloalkyl, —C₂alk-C₁haloalkyl, —C₃alk-C₁haloalkyl, —C₄alk-C₁haloalkyl, —C₅alk-C₁haloalkyl, —C₀alk-C₁haloalkyl, —C₀alk-C₂haloalkyl, —C₁alk-C₂haloalkyl, —C₂alk-C₂haloalkyl, —C₃alk-C₂haloalkyl, —C₄alk-C₂haloalkyl, —C₅alk-C₂haloalkyl, —C₆alk-C₂haloalkyl, —C₀alk-C₃haloalkyl, —C₁alk-C₃haloalkyl, —C₂alk-C₃haloalkyl, —C₃alk-C₃haloalkyl, —C₄alk-C₃haloalkyl, —C₅alk-C₃haloalkyl, —C₆alk-C₃haloalkyl, —C₀alk-C₄haloalkyl, —C₁alk-C₄haloalkyl, —C₂alk-C₄haloalkyl, —C₃alk-C₄haloalkyl, —C₄alk-C₄haloalkyl, —C₅alk-C₄haloalkyl, —C₆alk-C₄haloalkyl, —C₀alk-C₅haloalkyl, —C₁alk-C₅haloalkyl, —C₂alk-C₅haloalkyl, —C₃alk-C₅haloalkyl, —C₄alk-C₅haloalkyl, —C₅alk-C₅haloalkyl, —C₆alk-C₅haloalkyl, —C₀alk-C₆haloalkyl, —C₁alk-C₆haloalkyl, —C₂alk-C₆haloalkyl, —C₃alk-C₆haloalkyl, —C₄alk-C₆haloalkyl, —C₅alk-C₆haloalkyl, —C₆alk-C₆haloalkyl, fluoromethyl, fluoroethyl, fluoropropyl, fluorobutyl, fluoropentyl, chloromethyl, chloroethyl, chloropropyl, chlorobutyl, chloropentyl, bromomethyl, bromoethyl, bromopropyl, bromobutyl, bromopentyl, iodomethyl, iodoethyl, iodopropyl, iodobutyl, iodopentyl, —CH(OH)—C₁-C₆haloalkyl, —CH(F)—C₁-C₆haloalkyl, —CH(NH₂)—C₁-C₆haloalkyl, —CH(Me)-C₁-C₆haloalkyl, —C(Me)(OH)—C₁-C₆haloalkyl, and the like.

In some aspects, R¹ in Formula I or Formula II is —C₀-C₆alk-C≡CH, for example, —C₀alk-C≡CH, —C₁alk-C≡CH, —C₂alk-C≡CH, —C₃alk-C≡CH, —C₄alk-C≡CH, —C₅alk-C≡CH, —C₆alk-C≡CH, ethynyl, propargyl, —CH(OH)—C≡CH, —CH(F)—C≡CH, —CH(NH₂)—C≡CH, —CH(Me)-C≡CH, —C(Me)(OH)—C≡CH, and the like.

In some aspects, R¹ in Formula I or Formula II is —C₀-C₆alk-C≡C—C₁-C₆alkyl, for example, —C₀alk-C≡C—C₁alkyl, —C₁alk-C≡C—C₁alkyl, —C₂alk-C≡C—C₁alkyl, —C₃alk-C≡C—C₁alkyl, —C₄alk-C≡C—C₁alkyl, —C₅alk-C≡C—C₁alkyl, —C₀alk-C≡C—C₁alkyl, —C₀alk-C≡C—C₂alkyl, —C₁alk-C≡C—C₂alkyl, —C₂alk-C≡C—C₂alkyl, —C₃alk-C≡C—C₂alkyl, —C₄alk-C≡C—C₂alkyl, —C₅alk-C≡C—C₂alkyl, —C₆alk-C≡C—C₂alkyl, —C₀alk-C≡C—C₃alkyl, —C₁alk-C≡C—C₃alkyl, —C₂alk-C≡C—C₃alkyl, —C₃alk-C≡C—C₃alkyl, —C₄alk-C≡C—C₃alkyl, —C₅alk-C≡C—C₃alkyl, —C₆alk-C≡C—C₃alkyl, —C₀alk-C≡C—C₄alkyl, —C₁alk-C≡C—C₄alkyl, —C₂alk-C≡C—C₄alkyl, —C₃alk-C≡C—C₄alkyl, —C₄alk-C≡C—C₄alkyl, —C₅alk-C≡C—C₄alkyl, —C₆alk-C≡C—C₄alkyl, —C₀alk-C≡C—C₅alkyl, —C₁alk-C≡C—C₅alkyl, —C₂alk-C≡C—C₅alkyl, —C₃alk-C≡C—C₅alkyl, —C₄alk-C≡C—C₅alkyl, —C₅alk-C≡C—C₅alkyl, —C₆alk-C≡C—C₅alkyl, —C₀alk-C≡C—C₆alkyl, —C₁alk-C≡C—C₆alkyl, —C₂alk-C≡C—C₆alkyl, —C₃alk-C≡C—C₆alkyl, —C₄alk-C≡C—C₆alkyl, —C₅alk-C≡C—C₆alkyl, —C₆alk-C≡C—C₆alkyl, propynyl, butynyl, —CH(OH)—C≡C—C₁-C₆alkyl, —CH(F)—C≡C—C₁-C₆alkyl, —CH(NH₂)—C≡C—C₁-C₆alkyl, —CH(Me)-C≡C—C₁-C₆alkyl, —C(Me)(OH)—C≡C—C₁-C₆alkyl, and the like. In some embodiments wherein —C₀-C₆alk-C≡C—C₁-C₆alkyl is —C₀-C₆alk-C≡C—CH₃, R¹ is —CH(OH)—C≡C—CH₃, —CH(F)—C≡C—CH₃, —CH(NH₂)—C≡C—CH₃, —CH(Me)-C≡C—CH₃, or —C(Me)(OH)—C≡C—CH₃. Thus, in some embodiments, R¹ is —CH(OH)—C≡C—CH₃.

In some aspects, R¹ in Formula I or Formula II is —C₀-C₆alk-C≡C—C₁-C₆haloalkyl, for example, —C₀alk-C≡C—C₁haloalkyl, —C₁alk-C≡C—C₁haloalkyl, —C₂alk-C≡C—C₁haloalkyl, —C₃alk-C≡C—C₁haloalkyl, —C₄alk-C≡C—C₁haloalkyl, —C₅alk-C≡C—C₁haloalkyl, —C₀alk-C≡C—C₁haloalkyl, —C₀alk-C≡C—C₂haloalkyl, —C₁alk-C≡C—C₂haloalkyl, —C₂alk-C≡C—C₂haloalkyl, —C₃alk-C≡C—C₂haloalkyl, —C₄alk-C≡C—C₂haloalkyl, —C₅alk-C≡C—C₂haloalkyl, —C₆alk-C≡C—C₂haloalkyl, —C₀alk-C≡C—C₃haloalkyl, —C₁alk-C≡C—C₃haloalkyl, —C₂alk-C≡C—C₃haloalkyl, —C₃alk-C≡C—C₃haloalkyl, —C₄alk-C≡C—C₃haloalkyl, —C₅alk-C≡C—C₃haloalkyl, —C₆alk-C≡C—C₃haloalkyl, —C₀alk-C≡C—C₄haloalkyl, —C₁alk-C≡C—C₄haloalkyl, —C₂alk-C≡C—C₄haloalkyl, —C₃alk-C≡C—C₄haloalkyl, —C₄alk-C≡C—C₄haloalkyl, —C₅alk-C≡C—C₄haloalkyl, —C₆alk-C≡C—C₄haloalkyl, —C₀alk-C≡C—C₅haloalkyl, —C₁alk-C≡C—C₅haloalkyl, —C₂alk-C≡C—C₅haloalkyl, —C₃alk-C≡C—C₅haloalkyl, —C₄alk-C≡C—C₅haloalkyl, —C₅alk-C≡C—C₅haloalkyl, —C₆alk-C≡C—C₅haloalkyl, —C₀alk-C≡C—C₆haloalkyl, —C₁alk-C≡C—C₆haloalkyl, —C₂alk-C≡C—C₆haloalkyl, —C₃alk-C≡C—C₆haloalkyl, —C₄alk-C≡C—C₆haloalkyl, —C₅alk-C≡C—C₆haloalkyl, —C₆alk-C≡C—C₆haloalkyl, —CH(OH)—C≡C—C₁-C₆ haloalkyl, —CH(F)—C≡C—C₁-C₆ haloalkyl, —CH(NH₂)—C≡C—C₁-C₆ haloalkyl, —CH(Me)-C≡C—C₁-C₆ haloalkyl, —C(Me)(OH)—C≡C—C₁-C₆ haloalkyl, and the like. In some embodiments wherein —C₀-C₆alk-C≡C—C₁-C₆haloalkyl is —C₀-C₆alk-C—C—CF₃, R¹ is —CH(OH)—C≡C—CF₃, —CH(F)—C≡C—CF₃, —CH(NH₂)—C≡C—CF₃, —CH(Me)-C≡C—CF₃, —C(Me)(OH)—C≡C—CF₃, and the like. Thus, in some embodiments, R¹ is —CH(OH)—C≡C—CF₃.

In some aspects, R¹ in Formula I or Formula II is —C₀-C₆alk-C≡C—C₃-C₆cycloalkyl, for example, —C₀alk-C≡C—C₃cycloalkyl, —C₀alk-C≡C—C₄cycloalkyl, —C₀alk-C≡C—C₅cycloalkyl, —C₀alk-C≡C—C₆cycloalkyl, —C₁alk-C≡C—C₃cycloalkyl, —C₁alk-C≡C—C₄cycloalkyl, —C₁alk-C≡C—C₅-cycloalkyl, —C₁alk-C≡C—C₆cycloalkyl, —C₂alk-C≡C—C₃cycloalkyl, —C₂alk-C≡C—C₄cycloalkyl, —C₂alk-C≡C—C₅cycloalkyl, —C₂alk-C≡C—C₆cycloalkyl, —C₃alk-C≡C—C₃cycloalkyl, —C₃alk-C≡C—C₄cycloalkyl, —C₃alk-C≡C—C₅cycloalkyl, —C₃alk-C≡C—C₆cycloalkyl, —C₄alk-C≡C—C₃cycloalkyl, —C₄alk-C≡C—C₄cycloalkyl, —C₄alk-C≡C—C₅cycloalkyl, —C₄alk-C≡C—C₆cycloalkyl, —C₅alk-C≡C—C₃cycloalkyl, —C₅alk-C≡C—C₄cycloalkyl, —C₅alk-C≡C—C₅cycloalkyl, —C₅alk-C≡C—C₆cycloalkyl, —C₆alk-C≡C—C₃cycloalkyl, —C₆alk-C≡C—C₄cycloalkyl, —C₆alk-C≡C—C₅cycloalkyl, —C₆alk-C≡C—C₆cycloalkyl, —CH(OH)—C≡C—C₃-C₆cycloalkyl, —CH(F)—C≡C—C₃-C₆cycloalkyl, —CH(NH₂)—C≡C—C₃-C₆cycloalkyl, —CH(Me)-C≡C—C₃-C₆cycloalkyl, or —C(Me)(OH)—C≡C—C₃-C₆cycloalkyl. In some embodiments wherein —C₀-C₆alk-C≡C—C₃-C₆cycloalkyl is —C₀-C₆alk-C≡C-cyclopropyl, R¹ is —CH(OH)—C≡C-cyclopropyl, —CH(F)—C≡C— cyclopropyl, —CH(NH₂)—C≡C-cyclopropyl, —CH(Me)-C≡C-cyclopropyl, —C(Me)(OH)—C≡C— cyclopropyl, and the like. Thus, in some embodiments, R¹ is —CH(OH)—C≡C-cyclopropyl.

In some aspects, R¹ in Formula I or Formula II is —C₁-C₆alk-aryl, for example, —C₁alk-aryl, —C₂alk-aryl, —C₃alk-aryl, —C₄alk-aryl, —C₅alk-aryl, —C₀alk-aryl, —CH(OH)-aryl, —C(OCH₃)-aryl, —CH(F)-aryl, —CH(NH₂)-aryl, —CH(Me)-aryl, —C(Me)(OH)-aryl, —C(CF₃)(OH)-aryl, and the like. In some embodiments wherein R¹ is —C₁-C₆alk-aryl, the -aryl is -4-chlorophenyl, -3,4-dichlorophenyl, -3,4-difluorophenyl, -3-fluoro-4-chlorophenyl, -3-chloro-4-fluorophenyl, 4-(trifluoromethyl)phenyl, 3-fluoro-4-(trifluoromethyl)phenyl, -3-methyl-4-chlorophenyl, 2,3-dihydrobenzofuran-5-yl, 3-methyl-4-(trifluoromethyl)phenyl, or -benzo[d][1,3]dioxol-5-yl. Thus in some embodiments, R¹ is —CH(OH)-4-chlorophenyl, —CH(OH)-3,4-dichlorophenyl, —CH(OH)-3,4-difluorophenyl, —CH(OH)-3-fluoro-4-chlorophenyl, —CH(OH)-3-chloro-4-fluorophenyl, —CH(OH)-4-(trifluoromethyl)phenyl, —CH(OH)-3-fluoro-4-(trifluoromethyl)phenyl, —CH(OH)-2,3-dihydrobenzofuran-5-yl, —CH(OH)-3-methyl-4-(trifluoromethyl)phenyl, —CH(OH)-benzo[d][1,3]dioxol-5-yl, —C(CF₃)(OH)-4-chlorophenyl, —CH(OH)-3-methyl-4-chlorophenyl, —CH(F)-4-chlorophenyl, —CH(F)-3,4-dichlorophenyl, —CH(F)-3,4-difluorophenyl, —CH(F)-3-fluoro-4-chlorophenyl, —CH(F)-3-chloro-4-fluorophenyl, —CH(F)-4-(trifluoromethyl)phenyl, —CH(F)-3-fluoro-4-(trifluoromethyl)phenyl, —C(CF₃)(F)-4-chlorophenylphenyl, —CH(F)-3-methyl-4-chlorophenyl, —CH(F)-2,3-dihydrobenzofuran-5-yl, —CH(F)-3-methyl-4-(trifluoromethyl)phenyl, —CH(F)-benzo[d][1,3]dioxol-5-yl, —CH(NH₂)-4-chlorophenyl, —CH(NH₂)-3,4-dichlorophenyl, —CH(NH₂)-3,4-difluorophenyl, —CH(NH₂)-3-fluoro-4-chlorophenyl, —CH(NH₂)-3-chloro-4-fluorophenyl, —CH(NH₂)-4-(trifluoromethyl)phenyl, —CH(NH₂)-3-fluoro-4-(trifluoromethyl)phenyl, —C(CF₃)(NH₂)-4-chlorophenylphenyl, CH(NH₂)-3-methyl-4-chlorophenyl, —CH(NH₂)-2,3-dihydrobenzofuran-5-yl, —CH(NH₂)-3-methyl-4-(trifluoromethyl)phenyl, —CH(NH₂)-benzo[d][1,3]dioxol-5-yl, —CH(Me)-4-chlorophenyl, —CH(Me)-3,4-dichlorophenyl, —CH(Me)-3,4-difluorophenyl, —CH(Me)-3-fluoro-4-chlorophenyl, —CH(Me)-3-chloro-4-fluorophenyl, —CH(Me)-4-(trifluoromethyl)phenyl, —CH(Me)-3-fluoro-4-(trifluoromethyl)phenyl, —CH(Me)-3-methyl-4-chlorophenyl, —CH(Me)-2,3-dihydrobenzofuran-5-yl, —CH(Me)-3-methyl-4-(trifluoromethyl)phenyl, —CH(Me)-benzo[d][1,3]dioxol-5-yl, —C(CF₃)(Me)-4-chlorophenylphenyl, —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, or —C(Me)(OH)-3-chloro-4-fluorophenyl, —C(Me)(OH)-4-(trifluoromethyl)phenyl, —C(Me)(OH)-3-fluoro-4-(trifluoromethyl)phenyl, —C(Me)(OH)-3-methyl-4-chlorophenyl, —C(Me)(OH)-2,3-dihydrobenzofuran-5-yl, —C(Me)(OH)-benzo[d][1,3]dioxol-5-yl, —C(Me)(OH)-3-methyl-4-(trifluoromethyl)phenyl, —CH(OCH₃)-4-chlorophenyl, —CH(OCH₃)-3,4-dichlorophenyl, —CH(OCH₃)-3,4-difluorophenyl, —CH(OCH₃)-3-fluoro-4-chlorophenyl, —CH(OCH₃)-3-chloro-4-fluorophenyl, —CH(OCH₃)-4-(trifluoromethyl)phenyl, —CH(OCH₃)-3-fluoro-4-(trifluoromethyl)phenyl, —CH(OCH₃)-2,3-dihydrobenzofuran-5-yl, —CH(OCH₃)-benzo[d][1,3]dioxol-5-yl, —CH(OCH₃)-3-methyl-4-(trifluoromethyl)phenyl, —C(CF₃)(OCH₃)-4-chlorophenyl, —CH(OCH₃)-3-methyl-4-chlorophenyl.

In some aspects, R¹ in Formula I or Formula II is —C₁-C⁶alk-S—C₁-C₆alkyl, for example —C₁alk-S—C₁alkyl, —C₂alk-S—C₁alkyl, —C₃alk-S—C₁alkyl, —C₄alk-S—C₁alkyl, —C₅alk-S—C₁alkyl, —C₆alk-S—C₁alkyl, —C₁alk-S—C₂alkyl, —C₂alk-S—C₂alkyl, —C₃alk-S—C₂alkyl, —C₄alk-S—C₂alkyl, —C₅alk-S—C₂alkyl, —C₆alk-S—C₂alkyl, —C₁alk-S—C₃alkyl, —C₂alk-S—C₃alkyl, —C₃alk-S—C₃alkyl, —C₄alk-S—C₃alkyl, —C₅alk-S—C₃alkyl, —C₆alk-S—C₃alkyl, —C₁alk-S—C₄alkyl, —C₂alk-S—C₄alkyl, —C₃alk-S—C₄alkyl, —C₄alk-S—C₄alkyl, —C₅alk-S—C₄alkyl, —C₆alk-S—C₄alkyl, —C₁alk-S—C₅alkyl, —C₂alk-S—C₅alkyl, —C₃alk-S—C₅alkyl, —C₄alk-S—C₅alkyl, —C₅alk-S—C₅alkyl, —C₆alk-S—C₅alkyl, —C₁alk-S—C₆alkyl, —C₂alk-S—C₆alkyl, —C₃alk-S—C₆alkyl, —C₄alk-S—C₆alkyl, —C₅alk-S—C₆alkyl, —C₆alk-S—C₆alkyl, —CH₂S—C₂alkyl, —CH₂S—C₃alkyl, —CH₂S—C₄alkyl, —CH₂S—C₅alkyl, —CH₂S—C₆alkyl, and the like. Thus, in some aspects R¹ is —CH₂S—C₁alkyl. In some aspects, R¹ is —CH₂—S—CH₃.

In some aspects, R¹ in Formula I or Formula II is —C₁-C₆alk-S—C₁-C₆haloalkyl, for example —C₁alk-S—C₁haloalkyl, —C₂alk-S—C₁haloalkyl, —C₃alk-S—C₁haloalkyl, —C₄alk-S—C₁haloalkyl, —C₅alk-S—C₁haloalkyl, —C₆alk-S—C₁haloalkyl, —C₁alk-S—C₂haloalkyl, —C₂alk-S—C₂haloalkyl, —C₃alk-S—C₂haloalkyl, —C₄alk-S—C₂haloalkyl, —C₅alk-S—C₂haloalkyl, —C₆alk-S—C₂haloalkyl, —C₁alk-S—C₃haloalkyl, —C₂alk-S—C₃haloalkyl, —C₃alk-S—C₃haloalkyl, —C₄alk-S—C₃haloalkyl, —C₅alk-S—C₃haloalkyl, —C₆alk-S—C₃haloalkyl, —C₁alk-S—C₄haloalkyl, —C₂alk-S—C₄haloalkyl, —C₃alk-S—C₄haloalkyl, —C₄alk-S—C₄haloalkyl, —C₅alk-S—C₄haloalkyl, —C₆alk-S—C₄haloalkyl, —C₁alk-S—C₅haloalkyl, —C₂alk-S—C₅haloalkyl, —C₃alk-S—C₅haloalkyl, —C₄alk-S—C₅haloalkyl, —C₅alk-S—C₅haloalkyl, —C₆alk-S—C₅haloalkyl, —C₁alk-S—C₆haloalkyl, —C₂alk-S—C₆haloalkyl, —C₃alk-S—C₆haloalkyl, —C₄alk-S—C₆haloalkyl, —C₅alk-S—C₆haloalkyl, —C₆alk-S—C₆haloalkyl, —CH₂S—C₁haloalkyl, —CH₂S—C₂haloalkyl, —CH₂S—C₃haloalkyl, —CH₂S—C₄haloalkyl, —CH₂S—C₅haloalkyl, and —CH₂S—C₆haloalkyl.

In some aspects, R¹ in Formula I or Formula II is —C₁-C₆alk-S—C₃-C₆cycloalkyl, for example —C₁alk-S—C₃cycloalkyl, —C₂alk-S—C₃cycloalkyl, —C₃alk-S—C₃cycloalkyl, —C₄alk-S—C₃cycloalkyl, —C₅alk-S—C₃cycloalkyl, —C₆alk-S—C₃cycloalkyl, —C₁alk-S—C₄cycloalkyl, —C₂alk-S—C₄cycloalkyl, —C₃alk-S—C₄cycloalkyl, —C₄alk-S—C₄cycloalkyl, —C₅alk-S—C₄cycloalkyl, —C₆alk-S—C₄cycloalkyl, —C₁alk-S—C₅cycloalkyl, —C₂alk-S—C₅cycloalkyl, —C₃alk-S—C₅cycloalkyl, —C₄alk-S—C₅cycloalkyl, —C₅alk-S—C₅cycloalkyl, —C₆alk-S—C₅cycloalkyl, —C₁alk-S—C₆cycloalkyl, —C₂alk-S—C₆cycloalkyl, —C₃alk-S—C₆cycloalkyl, —C₄alk-S—C₆cycloalkyl, —C₅alk-S—C₆cycloalkyl, —C₆alk-S—C₆cycloalkyl, —CH₂S—C₃cycloalkyl, —CH₂S—C₄cycloalkyl, —CH₂S—C₅cycloalkyl, —CH₂S—C₆cycloalkyl, and the like.

In some aspects, R¹ in Formula I or Formula II is —C₁-C₆alk-S—C₃-C₆halocycloalkyl, for example —C₁alk-S—C₃halocycloalkyl, —C₂alk-S—C₃halocycloalkyl, —C₃alk-S—C₃halocycloalkyl, —C₄alk-S—C₃halocycloalkyl, —C₅alk-S—C₃halocycloalkyl, —C₆alk-S—C₃halocycloalkyl, —C₁alk-S—C₄halocycloalkyl, —C₂alk-S—C₄halocycloalkyl, —C₃alk-S—C₄halocycloalkyl, —C₄alk-S—C₄halocycloalkyl, —C₅alk-S—C₄halocycloalkyl, —C₆alk-S—C₄halocycloalkyl, —C₁alk-S—C₅halocycloalkyl, —C₂alk-S—C₅halocycloalkyl, —C₃alk-S—C₅halocycloalkyl, —C₄alk-S—C₅halocycloalkyl, —C₅alk-S—C₅halocycloalkyl, —C₆alk-S—C₅halocycloalkyl, —C₁alk-S—C₆halocycloalkyl, —C₂alk-S—C₆halocycloalkyl, —C₃alk-S—C₆halocycloalkyl, —C₄alk-S—C₆halocycloalkyl, —C₅alk-S—C₆halocycloalkyl, —C₆alk-S—C₆halocycloalkyl, —CH₂S—C₃halocycloalkyl, —CH₂S—C₄halocycloalkyl, —CH₂S—C₅halocycloalkyl, —CH₂S—C₆halocycloalkyl, and the like.

In some aspects, R¹ in Formula I or Formula II is —C₁-C₆alk-OC₁-C₆alkyl, for example, —C₁alk-O—C₁alkyl, —C₂alk-O—C₁alkyl, —C₃alk-O—C₁alkyl, —C₄alk-O—C₁alkyl, —C₅alk-O—C₁alkyl, —C₆alk-O—C₁alkyl, —C₁alk-O—C₂alkyl, —C₂alk-O—C₂alkyl, —C₃alk-O—C₂alkyl, —C₄alk-O—C₂alkyl, —C₅alk-O—C₂alkyl, —C₆alk-O—C₂alkyl, —C₁alk-O—C₃alkyl, —C₂alk-O—C₃alkyl, —C₃alk-O—C₃alkyl, —C₄alk-O—C₃alkyl, —C₅alk-O—C₃alkyl, —C₆alk-O—C₃alkyl, —C₁alk-O—C₄alkyl, —C₂alk-O—C₄alkyl, —C₃alk-O—C₄alkyl, —C₄alk-O—C₄alkyl, —C₅alk-O—C₄alkyl, —C₆alk-O—C₄alkyl, —C₁alk-O—C₅alkyl, —C₂alk-O—C₅alkyl, —C₃alk-O—C₅alkyl, —C₄alk-O—C₅alkyl, —C₅alk-O—C₅alkyl, —C₆alk-O—C₅alkyl, —C₁alk-O—C₆alkyl, —C₂alk-O—C₆alkyl, —C₃alk-O—C₆alkyl, —C₄alk-O—C₆alkyl, —C₅alk-O—C₆alkyl, —C₆alk-O—C₆alkyl, —CH₂OC₁alkyl, —CH₂OC₂alkyl, —CH₂OC₃alkyl, —CH₂OC₄alkyl, —CH₂OC₅alkyl, —CH₂OC₆alkyl, and the like.

In some aspects, R¹ in Formula I or Formula II is —C₁-C₆alk-O—C₃-C₆cycloalkyl, for example, —C₁alk-O—C₃cycloalkyl, —C₂alk-O—C₃cycloalkyl, —C₃alk-O—C₃cycloalkyl, —C₄alk-O—C₃cycloalkyl, —C₅alk-O—C₃cycloalkyl, —C₆alk-O—C₃cycloalkyl, —C₁alk-O—C₄cycloalkyl, —C₂alk-O—C₄cycloalkyl, —C₃alk-O—C₄cycloalkyl, —C₄alk-O—C₄cycloalkyl, —C₅alk-O—C₄cycloalkyl, —C₆alk-O—C₄cycloalkyl, —C₁alk-O—C₅cycloalkyl, —C₂alk-O—C₅cycloalkyl, —C₃alk-O—C₅cycloalkyl, —C₄alk-O—C₅cycloalkyl, —C₅alk-O—C₅cycloalkyl, —C₆alk-O—C₅cycloalkyl, —C₁alk-O—C₆cycloalkyl, —C₂alk-O—C₆cycloalkyl, —C₃alk-O—C₆cycloalkyl, —C₄alk-O—C₆cycloalkyl, —C₅alk-O—C₆cycloalkyl, —C₆alk-O—C₆cycloalkyl, —CH₂O—C₆cycloalkyl, —CH₂O—C₅cycloalkyl, —CH₂O—C₄cycloalkyl, —CH₂O—C₃cycloalkyl, and the like.

In some aspects, R¹ in Formula I or Formula II is —C₁-C₆alk-SCH₂-aryl, for example —C₁alk-SCH₂-aryl, —C₂alk-SCH₂-aryl, —C₃alk-SCH₂-aryl, —C₄alk-SCH₂-aryl, —C₅alk-SCH₂-aryl, —C₆alk-SCH₂-aryl, —CH₂SCH₂-phenyl, —CH₂SCH₂-naphthyl, —CH₂SCH₂-fluorophenyl, —CH₂SCH₂-difluorophenyl, —CH₂SCH₂-fluoronaphthyl, —CH₂SCH₂-chlorophenyl, —CH₂SCH₂-bromophenyl, —CH₂SCH₂-iodophenyl, —CH₂SCH₂-methylphenyl, —CH₂SCH₂-4-chlorophenyl, —CH₂SCH₂-3,4-dichlorophenyl, —CH₂SCH₂-3,4-difluorophenyl, —CH₂SCH₂-3-fluoro-4-chlorophenyl, —CH₂SCH₂-3-chloro-4-fluorophenyl, and the like. Thus, in some aspects R¹ is —CH₂SCH₂-phenyl.

In some aspects, R¹ in Formula I or Formula II is —C₁-C₆alk-C(O)NH-aryl, for example, —C₁alk-C(O)NH-aryl, —C₂alk-C(O)NH-aryl, —C₃alk-C(O)NH-aryl, —C₄alk-C(O)NH-aryl, —C₅alk-C(O)NH-aryl, —C₆alk-C(O)NH-aryl, —CH₂C(O)NH-phenyl, —CH₂C(O)NH-naphthyl, —CH₂C(O)NH— fluorophenyl, —CH₂C(O)NH-difluorophenyl, —CH₂C(O)NH-fluoronaphthyl, —CH₂C(O)NH— chlorophenyl, —CH₂C(O)NH-bromophenyl, —CH₂C(O)NH-iodophenyl, —CH₂C(O)NH-methylphenyl, —CH₂C(O)NH-4-chlorophenyl, —CH₂C(O)NH-3,4-dichlorophenyl, —CH₂C(O)NH-3,4-difluorophenyl, —CH₂C(O)NH-3-fluoro-4-chlorophenyl, —CH₂C(O)NH-3-chloro-4-fluorophenyl and the like. Thus, in some aspects R¹ is —CH₂C(O)NH-phenyl.

In some aspects, R¹ in Formula I or Formula II is —C₀-C₆alk-heteroaryl, for example, —C₀alk-heteroaryl, —C₁alk-heteroaryl, —C₂alk-heteroaryl, —C₃alk-heteroaryl, —C₄alk-heteroaryl, —C₅alk-heteroaryl, and —C₀alk-heteroaryl. In some aspects, R¹ is 2-(2-amino-3-bromoquinolin-7-yl)ethyl, 2-(2-amino-3-chloroquinolin-7-yl)ethyl, 2-(2-((cyclopropylmethyl)amino)quinolin-7-yl)ethyl, 2-(2-(methylamino)quinolin-7-yl)ethyl, or 2-(2-aminoquinolin-7-yl)ethyl.

In some aspects, R¹ in Formula I or Formula II is —C₁-C₆alk-O-heteroaryl, for example, —C₁alk-O-heteroaryl, —C₂alk-O-heteroaryl, —C₃alk-O-heteroaryl, —C₄alk-O-heteroaryl, —C₅alk-O-heteroaryl, and —C₀alk-O-heteroaryl. In some aspects, R¹ is ((2-amino-3-bromoquinolin-7-yl)oxy)methyl.

In some aspects, R¹ in Formula I or Formula II is —C₁-C₆alk-S-heteroaryl, for example, —C₁alk-S-heteroaryl, —C₂alk-S-heteroaryl, —C₃alk-S-heteroaryl, —C₄alk-S-heteroaryl, —C₅alk-S-heteroaryl, and —C₀alk-S-heteroaryl. In some aspects, R¹ is ((2-amino-3-bromoquinolin-7-yl)thio)methyl.

In some aspects, R¹ in Formula I or Formula II is —C₁-C₆alk-NH-heteroaryl, for example, —C₁alk-NH-heteroaryl, —C₂alk-NH-heteroaryl, —C₃alk-NH-heteroaryl, —C₄alk-NH-heteroaryl, —C₅alk-NH-heteroaryl, and —C₀alk-NH-heteroaryl. In some aspects, R¹ is ((2-amino-3-bromoquinolin-7-yl)amino)methyl.

In some embodiments of Formula I or Formula II, R² is H, —C₁-C₆alkyl, —C₁-C₆haloalkyl, or —C₀-C₆alk-C₃-C₆cycloalkyl. Thus, in some embodiments, R² is H.

It will be apparent that when R² is H, the compounds of Formula I or Formula II may exist as tautomers having (E)- or (Z)-geometry at the exocyclic carbon-nitrogen double bond The compounds of Formula I and Formula II described and claimed herein are meant to encompass all such tautomers and geometric isomers. The depiction of a particular tautomer or geometric isomer is not intended to be limiting. Thus, when R² is H, compounds of Formula I may be represented by any of the following equivalent structures:

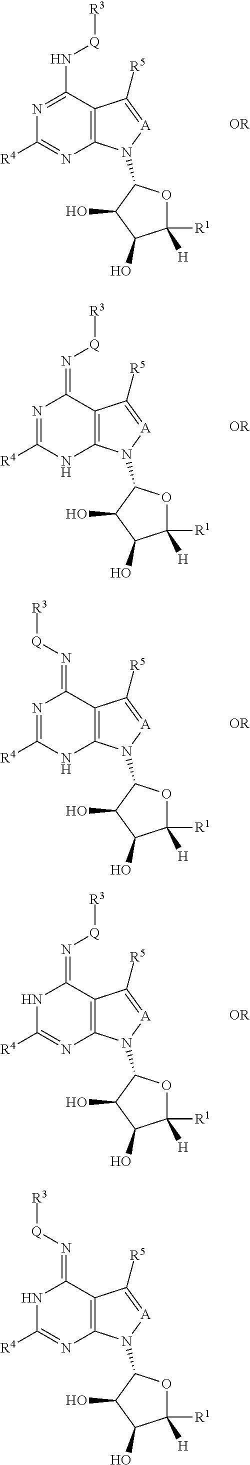

Similarly, when R² is H, compounds of Formula II may be represented by any of the following equivalent structures:

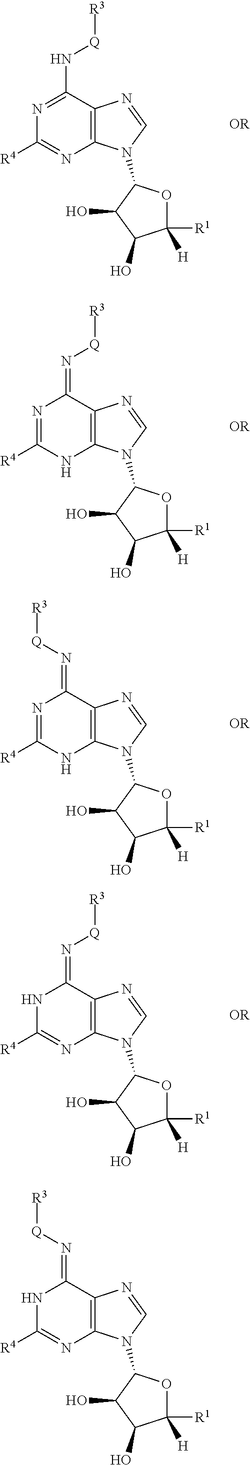

In some embodiments, R² in Formula I or Formula II is —C₁-C₆alkyl, for example, methyl, ethyl, propyl, isopropyl, butyl, isobutyl, s-butyl, t-butyl, pentyl, and the like. Thus, in some embodiments, R² is methyl (i.e., —CH₃, or Me).

In some aspects, R² in Formula I or Formula II is —C₁-C₆haloalkyl, for example, —CF₃ or —CHF₂ and the like.

In some aspects, R² in Formula I or Formula II is —C₀-C₆alk-C₃-C₆cycloalkyl, for example, —C₀alk-C₃cycloalkyl, —C₁alk-C₃cycloalkyl, —C₂alk-C₃cycloalkyl, —C₃alk-C₃cycloalkyl, —C₄alk-C₃cycloalkyl, —C₅alk-C₃cycloalkyl, —C₆alk-C₃cycloalkyl, —C₀alk-C₄cycloalkyl, —C₁alk-C₄cycloalkyl, —C₂alk-C₄cycloalkyl, —C₃alk-C₄cycloalkyl, —C₄alk-C₄cycloalkyl, —C₅alk-C₄cycloalkyl, —C₆alk-C₄cycloalkyl, —C₀alk-C⁵cycloalkyl, —C₁alk-C₅cycloalkyl, —C₂alk-C₅cycloalkyl, —C₃alk-C₅cycloalkyl, —C₄alk-C₅cycloalkyl, —C₅alk-C₅cycloalkyl, —C₆alk-C₅cycloalkyl, —C₀alk-C₆cycloalkyl, —C₁alk-C₆cycloalkyl, —C₂alk-C₆cycloalkyl, —C₃alk-C₆cycloalkyl, —C₄alk-C₆cycloalkyl, —C₅alk-C₆cycloalkyl, and —C₆alk-C₆cycloalkyl. In some aspects wherein R² is —C₀-C₆alk-C₃-C₆cycloalkyl, the cycloalkyl is unsubstituted. In other aspects wherein R² is —C₀-C₆alk-C₃-C₆cycloalkyl, the cycloalkyl is substituted with one, two, or three R substituents independently selected from C₁-C₆alkyl, (e.g., methyl, ethyl, propyl, isopropyl, butyl), —OC₁-C₆alkyl (e.g., —Omethyl, —Oethyl, —Opropyl, —Oisopropyl, —Obutyl), and halo (e.g., F or Cl).

In some aspects of Formula I and Formula II, R³ is H, —C₁-C₆alkyl, —C₁-C₆haloalkyl, —C₀-C₆alk-C₃-C₆cycloalkyl, —C(O)R⁷, —C(O)OR⁷, or —C(O)NR^{8a}R^{8b}. Thus, in some embodiments of Formula I or Formula II, R³ is H.

In some aspects, R³ in Formula I or Formula II is —C₁-C₆alkyl, for example, methyl, ethyl, propyl, isopropyl, butyl, isobutyl, s-butyl, t-butyl, pentyl, and the like. Thus, in some embodiments, R³ is methyl. In other embodiments, R³ is ethyl.

In some aspects, R³ in Formula I or Formula II is —C₁-C₆haloalkyl, for example, —CF₃, —CH₂CF₃, —CH₂CHF₂ or —CH₂ and the like.

In some aspects, R³ in Formula I or Formula II is —C₀-C₆alk-C₃-C₆cycloalkyl, for example, —C₀alk-C₃cycloalkyl, —C₁alk-C₃cycloalkyl, —C₂alk-C₃cycloalkyl, —C₃alk-C₃cycloalkyl, —C₄alk-C₃cycloalkyl, —C₅alk-C₃cycloalkyl, —C₆alk-C₃cycloalkyl, —C₀alk-C₄cycloalkyl, —C₁alk-C₄cycloalkyl, —C₂alk-C₄cycloalkyl, —C₃alk-C₄cycloalkyl, —C₄alk-C₄cycloalkyl, —C₅alk-C₄cycloalkyl, —C₆alk-C₄cycloalkyl, —C₀alk-C₅cycloalkyl, —C₁alk-C₅cycloalkyl, —C₂alk-C₅cycloalkyl, —C₃alk-C₅cycloalkyl, —C₄alk-C₅cycloalkyl, —C₅alk-C₅cycloalkyl, —C₆alk-C₅cycloalkyl, —C₀alk-C₆cycloalkyl, —C₁alk-C₆cycloalkyl, —C₂alk-C₆cycloalkyl, —C₃alk-C₆cycloalkyl, —C₄alk-C₆cycloalkyl, —C₅alk-C₆cycloalkyl, and —C₆alk-C₆cycloalkyl. In some aspects wherein R³ is —C₀-C₆alk-C₃-C₆cycloalkyl, the cycloalkyl is unsubstituted. In other aspects wherein R³ is —C₀-C₆alk-C₃-C₆cycloalkyl, the cycloalkyl is substituted with one, two, or three R substituents independently selected from C₁-C₆alkyl, (e.g., methyl, ethyl, propyl, isopropyl, butyl), —OC₁-C₆alkyl (e.g., —Omethyl, —Oethyl, —Opropyl, —Oisopropyl, —Obutyl), and halo (e.g., F or Cl).

In some embodiments, R³ in Formula I or Formula II is —C(O)R⁷ or —C(O)OR⁷. In these embodiments, R⁷ is H, C₁-C₆alkyl, or C₀-C₆alk-C₃-C₆cycloalkyl.

In some aspects, R⁷ in Formula I or Formula II is H, C₁-C₆alkyl, or C₀-C₆alk-C₃-C₆cycloalkyl. Thus, in some aspects, R⁷ is H.

In other aspects, R⁷ in Formula I or Formula II is C₁-C₆alkyl, for example, methyl, ethyl, propyl, isopropyl, butyl, isobutyl, s-butyl, t-butyl, pentyl, and the like. Thus, in some embodiments, R⁷ is methyl.

In other aspects, R⁷ in Formula I or Formula II is —C₀-C₆alk-C₃-C₆cycloalkyl, for example, —C₀alk-C₃cycloalkyl, —C₁alk-C₃cycloalkyl, —C₂alk-C₃cycloalkyl, —C₃alk-C₃cycloalkyl, —C₄alk-C₃cycloalkyl, —C₅alk-C₃cycloalkyl, —C₆alk-C₃cycloalkyl, —C₀alk-C₄cycloalkyl, —C₁alk-C₄cycloalkyl, —C₂alk-C₄cycloalkyl, —C₃alk-C₄cycloalkyl, —C₄alk-C₄cycloalkyl, —C₅alk-C₄cycloalkyl, —C₆alk-C₄cycloalkyl, —C₀alk-C₅cycloalkyl, —C₁alk-C₅cycloalkyl, —C₂alk-C₅cycloalkyl, —C₃alk-C₅cycloalkyl, —C₄alk-C₅cycloalkyl, —C₅alk-C₅cycloalkyl, —C₆alk-C₅cycloalkyl, —C₀alk-C₆cycloalkyl, —C₁alk-C₆cycloalkyl, —C₂alk-C₆cycloalkyl, —C₃alk-C₆cycloalkyl, —C₄alk-C₆cycloalkyl, —C₅alk-C₆cycloalkyl, and —C₆alk-C₆cycloalkyl.

In some aspects, R³ in Formula I or Formula II is —C(O)R⁷. In some embodiments wherein R⁷ is —C₁-C₆alkyl, R³ is —C(O)C₁-C₆alkyl. Thus, in some embodiments wherein R⁷ is methyl, R³ is acetyl (i.e., —C(O)CH₃).

In some aspects, R³ in Formula I or Formula II is —C(O)OR⁷. In some embodiments wherein R⁷ is —C₁-C₆alkyl, R³ is —C(O)OC₁-C₆alkyl. Thus, in some embodiments wherein R⁷ is methyl, R³ is —C(O)OCH₃.

In some aspects, R³ in Formula I or Formula II is or —C(O)NR^{8a}R^{8b}.

In some aspects, R^{8a} and R^{8b} in Formula I or Formula II are each independently H, C₁-C₆alkyl (e.g., methyl, ethyl, propyl, isopropyl, butyl, isobutyl, s-butyl, t-butyl, pentyl, and the like), or —C₀-C₆alk-O—C₁-C₆alkyl (e.g., —C₀alk-O—C₁alkyl, —C₁alk-O—C₁alkyl, —C₂alk-O—C₁alkyl, —C₃alk-O—C₁alkyl, —C₄alk-O—C₁alkyl, —C₅alk-O—C₁alkyl, —C₀alk-O—C₁alkyl, —C₀alk-O—C₂alkyl, —C₁alk-O—C₂alkyl, —C₂alk-O—C₂alkyl, —C₃alk-O—C₂alkyl, —C₄alk-O—C₂alkyl, —C₅alk-O—C₂alkyl, —C₆alk-O—C₂alkyl, —C₀alk-O—C₃alkyl, —C₁alk-O—C₃alkyl, —C₂alk-O—C₃alkyl, —C₃alk-O—C₃alkyl, —C₄alk-O—C₃alkyl, —C₅alk-O—C₃alkyl, —C₆alk-O—C₃alkyl, —C₀alk-O—C₄alkyl, —C₁alk-O—C₄alkyl, —C₂alk-O—C₄alkyl, —C₃alk-O—C₄alkyl, —C₄alk-O—C₄alkyl, —C₅alk-O—C₄alkyl, —C₆alk-O—C₄alkyl, —C₀alk-O—C₅alkyl, —C₁alk-O—C₅alkyl, —C₂alk-O—C₅alkyl, —C₃alk-O—C₅alkyl, —C₄alk-O—C₅alkyl, —C₅alk-O—C₅alkyl, —C₆alk-O—C₅alkyl, —C₀alk-O—C₆alkyl, —C₁alk-O—C₆alkyl, —C₂alk-O—C₆alkyl, —C₃alk-O—C₆alkyl, —C₄alk-O—C₆alkyl, —C₅alk-O—C₆alkyl, —C₆alk-O—C₆alkyl). In some embodiments, R^{8a} is C₁-C₆alkyl or —C₀-C₆alk-OC₁-C₆alkyl and R^{8b} is H, C₁-C₆alkyl, and —C₀-C₆alk-OC₁-C₆alkyl.

In some embodiments, R^{8a} in Formula I or Formula II is H or C₁-C₆alkyl. In some embodiments, R^{8b} is H or C₁-C₆alkyl. In some embodiments, R^{8a} and R^{8b} are each H. In other embodiments, R^{8a} and R^{8b} are each independently C₁-C₆alkyl. In some aspects, R^{8a} is C₁-C₆alkyl and R^{8b} is H.

In other aspects, R^{8a} and R^{8b} in Formula I or Formula II are each independently —C₀-C₆alk-OC₁-C₆alkyl.

In other aspects, R^{8a} in Formula I or Formula II is —C₀-C₆alk-OC₁-C₆alkyl and R^{8b} is H.

In yet other aspects, R^{8a} and R^{8b} in Formula I or Formula II, together with the atom to which they are attached, form a C₂-C₆heterocycloalkyl ring, for example, aziridinyl, azetidinyl, pyrrolidinyl, piperidinyl, and the like.

In some aspects of Formula I or Formula II, R⁴ is H, halo, C₁-C₆alkyl, or NH₂. Thus in some embodiments, R⁴ is H. In other embodiments, R⁴ is halo, for example, F, Cl, Br, or I, with Cl being preferred. In other embodiments, R⁴ is —C₁-C₆alkyl, for example, methyl, ethyl, propyl, isopropyl, butyl, isobutyl, s-butyl, t-butyl, pentyl, and the like. Thus, in some embodiments, R⁴ is methyl. In yet other embodiments, R⁴ is NH₂.

In embodiments of the disclosure wherein the compounds are of Formula I, R⁵ is H, halo, CN, —C₁-C₆alkyl, —C₂-C₄alkenyl, —C₂-C₄haloalkenyl, C₂-C₄cyanoalkenyl, —C₀-C₆alk-C≡CH, —C₀-C₆alk-C≡C—C₁-C₆alkyl, —C₁-C₄haloalkyl, —C₂-C₆heterocycloalkyl, oxo-substituted-C₂-C₆heterocycloalkyl, —C₃-C₆cycloalkyl, —C₀-C₃-alk-C(O)R⁹, —CR⁸R^{8′}CN, —CH₂NR′R^{8′}, —C₀-C₆alk-OH, —NR⁸R^{8′}, —N(R⁹)CN, —O—C₁-C₄alkyl, —NR⁹CONR⁸R^{8′}, —OCONR⁸R^{8′}, or —NR⁹C(O)OR^{9a}.

In some emodiments, R⁵ in the compounds of Formula I is H.

In some embodiments, R⁵ in the compounds of Formula I is halo, for example, F, Cl, Br, or I. Thus, in some embodiments, R⁵ is F.

In some emodiments, R⁵ in the compounds of Formula I is CN.

In other embodiments, R⁵ in the compounds of Formula I is —C₁-C₆alkyl, for example, methyl, ethyl, propyl, isopropyl, butyl, isobutyl, s-butyl, t-butyl, pentyl, and the like. Thus, in some aspects, R⁵ is methyl.

In some aspects, R⁵ in the compounds of Formula I is —C₂-C₄alkenyl, for example, vinyl, allyl, and the like. Thus, in some embodiments, R⁵ is vinyl (—CH═CH₂).

In some aspects, R⁵ in the compounds of Formula I is —C₂-C₄haloalkenyl, for example, —C(F)═CH₂, C(CF₃)═CH₂, and the like. Thus, in some embodiments, R⁵ is —C(F)═CH₂.

In other aspects, R⁵ is —C₂-C₄cyanoalkenyl, for example, —C(CN)═CH₂, —CH═CHCN, and the like. Thus, in some embodiments, R⁵ is —C(CN)═CH₂.

In other embodiments, R⁵ in the compounds of Formula I is —C₀-C₆alk-C≡CH, for example, —C₀alk-C≡CH, —C₁alk-C≡CH, —C₂alk-C≡CH, —C₃alk-C≡CH, —C₄alk-C≡CH, —C₅alk-C≡CH, —C₆alk-C≡CH, ethynyl, propargyl, and the like. Thus, in some embodiments, R⁵ is ethynyl (—C≡CH).

In some aspects, R⁵ in the compounds of Formula I is —C₀-C₆alk-C≡C—C₁-C₆alkyl, for example, —C₀alk-C≡C—C₁alkyl, —C₁alk-C≡C—C₁alkyl, —C₂alk-C≡C—C₁alkyl, —C₃alk-C≡C—C₁alkyl, —C₄alk-C≡C—C₁alkyl, —C₅alk-C≡C—C₁alkyl, —C₀alk-C≡C—C₁alkyl, —C₀alk-C≡C—C₂alkyl, —C₁alk-C≡C—C₂alkyl, —C₂alk-C≡C—C₂alkyl, —C₃alk-C≡C—C₂alkyl, —C₄alk-C≡C—C₂alkyl, —C₅alk-C≡C—C₂alkyl, —C₆alk-C≡C—C₂alkyl, —C₀alk-C≡C—C₃alkyl, —C₁alk-C≡C—C₃alkyl, —C₂alk-C≡C—C₃alkyl, —C₃alk-C≡C—C₃alkyl, —C₄alk-C≡C—C₃alkyl, —C₅alk-C≡C—C₃alkyl, —C₆alk-C≡C—C₃alkyl, —C₀alk-C≡C—C₄alkyl, —C₁alk-C≡C—C₄alkyl, —C₂alk-C≡C—C₄alkyl, —C₃alk-C≡C—C₄alkyl, —C₄alk-C≡C—C₄alkyl, —C₅alk-C≡C—C₄alkyl, —C₆alk-C≡C—C₄alkyl, —C₀alk-C≡C—C₅alkyl, —C₁alk-C≡C—C₅alkyl, —C₂alk-C≡C—C₅alkyl, —C₃alk-C≡C—C₅alkyl, —C₄alk-C≡C—C₅alkyl, —C₅alk-C≡C—C₅alkyl, —C₆alk-C≡C—C₅alkyl, —C₀alk-C≡C—C₆alkyl, —C₁alk-C≡C—C₆alkyl, —C₂alk-C≡C—C₆alkyl, —C₃alk-C≡C—C₆alkyl, —C₄alk-C≡C—C₆alkyl, —C₅alk-C≡C—C₆alkyl, and —C₆alk-C≡C—C₆alkyl.

In some embodiments, R⁵ in the compounds of Formula I is —C₁-C₄haloalkyl, for example, —CF₃ or —CH₂.

In some embodiments, R⁵ in the compounds of Formula I is —C₂-C₆heterocycloalkyl, for example C₂heterocycloalkyl, C₃heterocycloalkyl, C₄heterocycloalkyl, C₅heterocycloalkyl, and C₆heterocycloalkyl, including azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, tetrahydrofuranyl, tetrahydropyranyl, piperazinyl, and the like. Thus, in some embodiments, R⁵ is 2-oxiranyl. In other embodiments, R⁵ is 1-azetidinyl.

In some embodiments, R⁵ in the compounds of Formula I is oxo-substituted-C₂-C₆heterocycloalkyl, for example, oxo-substituted-C₂heterocycloalkyl, oxo-substituted-C₃heterocycloalkyl, oxo-substituted-C₄heterocycloalkyl, oxo-substituted-C₅heterocycloalkyl, oxo-substituted-C₆heterocycloalkyl, including aziridinonyl, azetidinonyl, pyrrolidinonyl, dioxolanonyl, imidazolidinonyl, pyrazolidinonyl, piperazinonyl, piperidinonyl, dioxanonyl, dithianonyl, thiomorpholinonyl, oxazepanonyl, oxiranonyl, oxetanonyl, quinuclidinonyl, tetrahydrofuranonyl, tetrahydropyranonyl, piperazinonyl, and the like. Thus, in some embodiments, R⁵ is azetidin-2-one-1-yl.

In some embodiments, R⁵ in the compounds of Formula I is —C₃-C₆cycloalkyl, for example —C₃cycloalkyl, —C₄cycloalkyl, —C₅cycloalkyl, —C₆cycloalkyl, and the like. In some embodiments, R⁵ is —C₃cycloalkyl. Thus, in some embodiments, R⁵ is cyclopropyl.

In other embodiments, R⁵ in the compounds of Formula I is —C₀-C₃-alk-C(O)R⁹, for example —C₀-alk-C(O)R⁹, —C₁-alk-C(O)R⁹, —C₂-alk-C(O)R⁹, and —C₃-alk-C(O)R⁹. In some embodiments wherein R⁹ is C₁-C₆alkyl, R⁵ is —C₀-C₃-alk-C(O)C₁-C₆alkyl. Thus, in some embodiments wherein R⁹ is methyl, R⁵ is acetyl (i.e., —C(O)CH₃). In some embodiments wherein R⁹ is —C₀-C₆alk-C₃-C₆cycloalkyl, R⁵ is —C(O)C₀-C₆alk-C₃-C₆cycloalkyl. In some embodiments wherein R⁹ is H, R⁵ is —CHO.

In some embodiments, R⁵ in the compounds of Formula I is —CR⁸R^{8′}CN. Thus, in some embodiments wherein R⁸ and R^{8′} are both H, R⁵ is cyanomethyl (i.e., —CH₂CN). In some embodiments wherein R⁸ is —C₁-C₆alkyl and R^{8′} is H, R⁵ is —CH(—C₁-C₆alkyl)CN. In some embodiments wherein R⁸ and R^{8′} are both —C₁-C₆alkyl, R⁵ is —C(C₁-C₆alkyl)(C₁-C₆alkyl)CN. In some embodiments wherein R⁸ is —C₀-C₆alk-OC₁-C₆alkyl and R^{8′} is H, R⁵ is —CH(—C₀-C₆alk-OC₁-C₆alkyl)CN. In some embodiments wherein R⁸ and R^{8′} are both —C₀-C₆alk-OC₁-C₆alkyl, R⁵ is —C(—C₀-C₆alk-OC₁-C₆alkyl)(—C₀-C₆alk-OC₁-C₆alkyl)CN.

In some embodiments, R⁵ in the compounds of Formula I is CH₂NR⁸R^{8′}. Thus, in some embodiments wherein R⁸ and R^{8′} are both H, R⁵ is aminomethyl (i.e., —CH₂NH₂). In some embodiments wherein R⁸ is —C₁-C₆alkyl and R^{8′} is H, R⁵ is —CH₂NH(C₁-C₆alkyl). In some embodiments wherein R⁸ and R^{8′} are both —C₁-C₆alkyl, R⁵ is —CH₂N(C₁-C₆alkyl)(C₁-C₆alkyl). In some embodiments wherein R⁸ is —C₀-C₆alk-OC₁-C₆alkyl and R^{8′} is H, R⁵ is —CH₂NH(—C₀-C₆alk-OC₁-C₆alkyl). In some embodiments wherein R⁸ and R^{8′} are both —C₀-C₆alk-OC₁-C₆alkyl, R⁵ is —CH₂N(—C₀-C₆alk-OC₁-C₆alkyl)(—C₀-C₆alk-OC₁-C₆alkyl).

In some embodiments, R⁵ in the compounds of Formula I is —C₀-C₆alk-OH, for example, —C₀alk-OH, —C₁alk-OH, —C₂alk-OH, —C₃alk-OH, —C₄alk-OH, —C₅alk-OH, —C₆alk-OH, and the like. In some embodiments R⁵ is —C₁alk-OH. In some embodiments, R⁵ is hydroxymethyl (i.e., CH₂OH). In other embodiments, R⁵ is hydroxyethyl (i.e., —CH₂CH₂OH).

In some embodiments, R⁵ in the compounds of Formula I is —NR⁸R^{8′}. Thus, in some embodiments wherein R⁸ and R^{8′} are both H, R⁵ is amino (i.e., —NH₂). In some embodiments wherein R⁸ is —C₁-C₆alkyl and R^{8′} is H, R⁵ is —NH(C₁-C₆alkyl). Thus, in some embodiments wherein R⁸ is methyl and R^{8′} is H, R⁵ is methylamino (i.e., —NHCH₃). In some embodiments wherein R⁸ and R^{8′} are both —C₁-C₆alkyl, R⁵ is —N(—C₁-C₆alkyl)(—C₁-C₆alkyl). In some embodiments wherein R⁸ is —C₀-C₆alk-OC₁-C₆alkyl and R^{8′} is H, R⁵ is —NH(—C₀-C₆alk-OC₁-C₆alkyl). In some embodiments wherein R⁸ and R^{8′} are both —C₀-C₆alk-OC₁-C₆alkyl, R⁵ is —N(—C₀-C₆alk-OC₁-C₆alkyl)(—C₀-C₆alk-OC₁-C₆alkyl).

In some embodiments, R⁵ in the compounds of Formula I is —N(R⁹)CN. In some embodiments wherein R⁹ is —C₁-C₆alkyl, R⁵ is —N(C₁-C₆alkyl)CN. Thus, in some embodiments wherein R⁹ is methyl, R⁵ is —N(CH₃)CN. In some embodiments wherein R⁹ is —C₀-C₆alk-C₃-C₆cycloalkyl, R⁵ is —N(—C₀-C₆alk-C₃-C₆cycloalkyl)CN. In some embodiments wherein R⁹ is H, R⁵ is —NH—CN.

In some embodiments, R⁵ in the compounds of Formula I is —O—C₁-C₄ alkyl, for example —O—C₁alkyl, —O—C₂alkyl, —O—C₃alkyl, —O—C₄alkyl.

In some embodiments, R⁵ in the compounds of Formula I is —NR⁹C(O)NR⁸R^{8′}. In some embodiments wherein R⁹ is H, R⁵ is —NHC(O)NR⁸R^{8′}. In some embodiments wherein R⁹ is —C₁-C₆alkyl, R⁵ is —N(—C₁-C₆alkyl)C(O)NR⁸R^{8′}. In some embodiments wherein R⁹ is C₀-C₆alk-C₃-C₆cycloalkyl, R⁵ is —N(C₀-C₆alk-C₃-C₆cycloalkyl)C(O)NR⁸R^{8′}. In some embodiments wherein R⁸ is H, R⁵ is —NR⁹C(O)NHR^{8′}. In some embodiments wherein R⁸ is H and R^{8′} is H, R⁵ is —NR⁹C(O)NH₂. Thus, in some embodiments wherein R⁹ is H and R⁸ and R^{8′} are both H, R⁵ is urea-1-yl (i.e., —NHC(O)NH₂). In some embodiments wherein R⁹ is —C₁-C₆alkyl and R^{8′} is H, R⁵ is —NR⁹C(O)NH(C₁-C₆alkyl). In some embodiments wherein R⁸ and R^{8′} are both —C₁-C₆alkyl, R⁵ is —NR⁹C(O)N(C₁-C₆alkyl)(C₁-C₆alkyl). In some embodiments wherein R⁸ is —C₀-C₆alk-OC₁-C₆alkyl and R^{8′} is H, R⁵ is —NR⁹C(O)NH(—C₀-C₆alk-OC₁-C₆alkyl). In some embodiments wherein R⁸ and R^{8′} are both —C₀-C₆alk-OC₁-C₆alkyl, R⁵ is —NR⁹C(O)N(—C₀-C₆alk-OC₁-C₆alkyl)(—C₀-C₆alk-OC₁-C₆alkyl).

In some embodiments, R⁵ in the compounds of Formula I is —OC(O)NR⁸R^{8′}. In some embodiments wherein R⁸ is H, R⁵ is —OC(O)NHR^{8′}. In some embodiments wherein R⁸ is H and R^{8′} is H, R⁵ is —OC(O)NH₂. In some embodiments wherein R⁸ is —C₁-C₆alkyl and R^{8′} is H, R⁵ is —OC(O)NH(C₁-C₆alkyl). In some embodiments wherein R⁸ and R^{8′} are both —C₁-C₆alkyl, R⁵ is —OC(O)N(C₁-C₆alkyl)(C₁-C₆alkyl). In some embodiments wherein R⁸ is —C₀-C₆alk-OC₁-C₆alkyl and R^{8′} is H, R⁵ is —OC(O)NH(—C₀-C₆alk-OC₁-C₆alkyl). In some embodiments wherein R⁸ and R^{8′} are both —C₀-C₆alk-OC₁-C₆alkyl, R⁵ is —OC(O)N(—C₀-C₆alk-OC₁-C₆alkyl)(—C₀-C₆alk-OC₁-C₆alkyl).

In some embodiments, R⁵ in the compounds of Formula I is —NR⁹C(O)OR^{9a}. In some embodiments wherein R⁹ is H, R⁵ is —NHC(O)OR^{9a}. In some embodiments wherein R⁹ is —C₁-C₆alkyl, R⁵ is —N(—C₁-C₆alkyl)C(O)OR^{9a}. In some embodiments wherein R⁹ is C₀-C₆alk-C₃-C₆cycloalkyl, R⁵ is —N(C₀-C₆alk-C₃-C₆cycloalkyl)C(O)OR^{9a}. In some embodiments wherein R^{9a} is —C₁-C₆alkyl, R⁵ is —NR⁹C(O)O—C₁-C₆alkyl. In some embodiments wherein R^{9a} is —C₀-C₆alk-C₃-C₆cycloalkyl, R⁵ is —NR⁹C(O)O—C₀-C₆alk-C₃-C₆cycloalkyl. In some embodiments wherein R⁹ is H and R^{9a} is —C₁-C₆alkyl, R⁵ is —NHC(O)O—C₁-C₆alkyl. Thus, in some embodiments wherein R⁹ is H and R^{9a} is methyl, R⁵ is —NHC(O)OCH₃.

In embodiments of the disclosure wherein the compounds are of Formula I or Formula II, R⁶ is —C₁-C₆alkyl or C₀-C₆alk-C₃-C₆cycloalkyl. In some embodiments, R⁶ is —C₁-C₆alkyl, for example, methyl, ethyl, propyl, isopropyl, butyl, isobutyl, s-butyl, t-butyl, pentyl, and the like. Thus, in some embodiments, R⁶ is methyl (i.e., —CH₃, or Me). In other embodiments, R⁶ is —C₀-C₆alk-C₃-C₆cycloalkyl, for example, —C₀alk-C₃cycloalkyl, —C₁alk-C₃cycloalkyl, —C₂alk-C₃cycloalkyl, —C₃alk-C₃cycloalkyl, —C₄alk-C₃cycloalkyl, —C₅alk-C₃cycloalkyl, —C₆alk-C₃cycloalkyl, —C₀alk-C₄cycloalkyl, —C₁alk-C₄cycloalkyl, —C₂alk-C₄cycloalkyl, —C₃alk-C₄cycloalkyl, —C₄alk-C₄cycloalkyl, —C₅alk-C₄cycloalkyl, —C₆alk-C₄cycloalkyl, —C₀alk-C₅cycloalkyl, —C₁alk-C₅cycloalkyl, —C₂alk-C₅cycloalkyl, —C₃alk-C₅cycloalkyl, —C₄alk-C₅cycloalkyl, —C₅alk-C₅cycloalkyl, —C₆alk-C₅cycloalkyl, —C₀alk-C₆cycloalkyl, —C₁alk-C₆cycloalkyl, —C₂alk-C₆cycloalkyl, —C₃alk-C₆cycloalkyl, —C₄alk-C₆cycloalkyl, —C₅alk-C₆cycloalkyl, and —C₆alk-C₆cycloalkyl.

In embodiments of the disclosure wherein the compounds are of Formula I, R⁸ and R^{8′} are each independently H, C₁-C₆alkyl, or —C₀-C₆alk-OC₁-C₆alkyl, or R⁸ and R^{8′}, together with the atom to which they are attached, form a C₂-C₆heterocycloalkyl ring or a C₃-C₆cycloalkyl ring.

In some aspects, R⁸ and R^{8′} in the compounds are of Formula I are each independently H, C₁-C₆alkyl (e.g., methyl, ethyl, propyl, isopropyl, butyl, isobutyl, s-butyl, t-butyl, pentyl, and the like), or —C₀-C₆alk-OC₁-C₆alkyl (e.g., —C₀alk-O—C₁alkyl, —C₁alk-O—C₁alkyl, —C₂alk-O—C₁alkyl, —C₃alk-O—C₁alkyl, —C₄alk-O—C₁alkyl, —C₅alk-O—C₁alkyl, —C₀alk-O—C₁alkyl, —C₀alk-O—C₂alkyl, —C₁alk-O—C₂alkyl, —C₂alk-O—C₂alkyl, —C₃alk-O—C₂alkyl, —C₄alk-O—C₂alkyl, —C₅alk-O—C₂alkyl, —C₆alk-O—C₂alkyl, —C₀alk-O—C₃alkyl, —C₁alk-O—C₃alkyl, —C₂alk-O—C₃alkyl, —C₃alk-O—C₃alkyl, —C₄alk-O—C₃alkyl, —C₅alk-O—C₃alkyl, —C₀alk-O—C₃alkyl, —C₀alk-O—C₄alkyl, —C₁alk-O—C₄alkyl, —C₂alk-O—C₄alkyl, —C₃alk-O—C₄alkyl, —C₄alk-O—C₄alkyl, —C₅alk-O—C₄alkyl, —C₀alk-O—C₄alkyl, —C₀alk-O—C₅alkyl, —C₁alk-O—C₅alkyl, —C₂alk-O—C₅alkyl, —C₃alk-O—C₅alkyl, —C₄alk-O—C₅alkyl, —C₅alk-O—C₅alkyl, —C₆alk-O—C₅alkyl, —C₀alk-O—C₆alkyl, —C₁alk-O—C₆alkyl, —C₂alk-O—C₆alkyl, —C₃alk-O—C₆alkyl, —C₄alk-O—C₆alkyl, —C₅alk-O—C₆alkyl, and —C₀alk-O—C₆alkyl). In some embodiments, R⁸ is C₁-C₆alkyl or —C₀-C₆alk-OC₁-C₆alkyl and R^{8′} is H, C₁-C₆alkyl, or —C₀-C₆alk-OC₁-C₆alkyl.

In some embodiments, R⁸ in the compounds of Formula I is H or C₁-C₆alkyl. In some embodiments, R^{8′} in the compounds of Formula I is H or C₁-C₆alkyl. In some embodiments, R¹ and R^{8′} are each H. In other embodiments, R⁸ and R^{8′} are each independently C₁-C₆alkyl. In some aspects, R⁸ is C₁-C₆alkyl and R^{8′} is H.

In other aspects, R⁸ and R^{8′} in the compounds of Formula I are each independently —C₀-C₆alk-OC₁-C₆alkyl.

In other aspects, R⁸ in the compounds of Formula I is —C₀-C₆alk-OC₁-C₆alkyl and R^{8′} is H.

In yet other aspects, R⁸ and R^{8′} in the compounds of Formula I, together with the atom to which they are attached, form a C₃-C₆cycloalkyl ring, for example, cyclopropyl, cyclobutyl, cyclopentyl, or cyclohexyl, and the like, or a C₂-C₆heterocycloalkyl ring, for example, aziridinyl, azetidinyl, pyrrolidinyl, piperidinyl, and the like.

In embodiments of the disclosure wherein the compounds are of Formula I, R⁹ is H, —C₁-C₆alkyl (e.g., methyl, ethyl, propyl, isopropyl, butyl, isobutyl, s-butyl, t-butyl, pentyl, and the like), or C₀-C₆alk-C₃-C₆cycloalkyl (e.g., —C₀alk-C₃cycloalkyl, —C₁alk-C₃cycloalkyl, —C₂alk-C₃cycloalkyl, —C₃alk-C₃cycloalkyl, —C₄alk-C₃cycloalkyl, —C₅alk-C₃cycloalkyl, —C₆alk-C₃cycloalkyl, —C₀alk-C₄cycloalkyl, —C₁alk-C₄cycloalkyl, —C₂alk-C₄cycloalkyl, —C₃alk-C₄cycloalkyl, —C₄alk-C₄cycloalkyl, —C₅alk-C₄cycloalkyl, —C₆alk-C₄cycloalkyl, —C₀alk-C₅cycloalkyl, —C₁alk-C₅cycloalkyl, —C₂alk-C₅cycloalkyl, —C₃alk-C₅cycloalkyl, —C₄alk-C₅cycloalkyl, —C₅alk-C₅cycloalkyl, —C₆alk-C₅cycloalkyl, —C₀alk-C₆cycloalkyl, —C₁alk-C₆cycloalkyl, —C₂alk-C₆cycloalkyl, —C₃alk-C₆cycloalkyl, —C₄alk-C₆cycloalkyl, —C₅alk-C₆cycloalkyl, and —C₀alk-C₆cycloalkyl).

In embodiments of the disclosure wherein the compounds are of Formula I, R^{9a} is —C₁-C₆alkyl, or C₀-C₆alk-C₃-C₆cycloalkyl. In some embodiments, R^{9a} is —C₁-C₆alkyl (e.g., methyl, ethyl, propyl, isopropyl, butyl, isobutyl, s-butyl, t-butyl, pentyl, and the like), or C₀-C₆alk-C₃-C₆cycloalkyl (e.g., —C₀alk-C₃cycloalkyl, —C₁alk-C₃cycloalkyl, —C₂alk-C₃cycloalkyl, —C₃alk-C₃cycloalkyl, —C₄alk-C₃cycloalkyl, —C₅alk-C₃cycloalkyl, —C₆alk-C₃cycloalkyl, —C₀alk-C₄cycloalkyl, —C₁alk-C₄cycloalkyl, —C₂alk-C₄cycloalkyl, —C₃alk-C₄cycloalkyl, —C₄alk-C₄cycloalkyl, —C₅alk-C₄cycloalkyl, —C₆alk-C₄cycloalkyl, —C₀alk-C₅cycloalkyl, —C₁alk-C₅cycloalkyl, —C₂alk-C₅cycloalkyl, —C₃alk-C₅cycloalkyl, —C₄alk-C₅cycloalkyl, —C₅alk-C₅cycloalkyl, —C₆alk-C₅cycloalkyl, —C₀alk-C₆cycloalkyl, —C₁alk-C₆cycloalkyl, —C₂alk-C₆cycloalkyl, —C₃alk-C₆cycloalkyl, —C₄alk-C₆cycloalkyl, —C₅alk-C₆cycloalkyl, and —C₆alk-C₆cycloalkyl).

In preferred embodiments of the compounds of Formula I or Formula II, R¹ is —C₀-C₆alk-C₁-C₆alkyl, —C₀-C₆alk-C₁-C₆haloalkyl, —C₀-C₆alk-C≡CH, —C₀-C₆alk-C≡C—C₁-C₆alkyl, —C₀-C₆alk-C≡C—C₁-C₆haloalkyl, —C₀-C₆alk-C≡C—C₃-C₆cycloalkyl, or —C₁-C₆alk-aryl.

More preferred embodiments of the compounds of Formula I or Formula II are those wherein R¹ is —CH(OH)—C₁-C₆alkyl, —CH(F)—C₁-C₆alkyl, —CH(NH₂)—C₁-C₆alkyl, —CH(Me)-C₁-C₆alkyl, —C(Me)(OH)—C₁-C₆alkyl, —CH(OH)—C₁-C₆ haloalkyl, —CH(F)—C₁-C₆ haloalkyl, —CH(NH₂)—C₁-C₆ haloalkyl, —CH(Me)-C₁-C₆ haloalkyl, —C(Me)(OH)—C₁-C₆ haloalkyl, —CH(OH)—C≡CH, —CH(F)—C≡CH, —CH(NH₂)—C≡CH, —CH(Me)-C≡CH, —C(Me)(OH)—C≡CH, —CH(OH)—C≡C—C₁-C₆alkyl, —CH(F)—C≡C—C₁-C₆alkyl, —CH(NH₂)—C≡C—C₁-C₆alkyl, —CH(Me)-C≡C—C₁-C₆alkyl, —C(Me)(OH)—C≡C—C₁-C₆alkyl, CH(OH)—C≡C—C₁-C₆haloalkyl, —CH(F)—C≡C—C₁-C₆haloalkyl, —CH(NH₂)—C≡C—C₁-C₆haloalkyl, —CH(Me)-C≡C— C₁-C₆haloalkyl, —C(Me)(OH)—C≡C—C₁-C₆haloalkyl, —CH(OH)—C≡C—C₃-C₆cycloalkyl, —CH(F)—C≡C—C₃-C₆cycloalkyl, —CH(NH₂)—C≡C—C₃-C₆cycloalkyl, —CH(Me)-C≡C—C₃-C₆cycloalkyl, —C(Me)(OH)—C≡C—C₃-C₆cycloalkyl, —CH(OH)-aryl, —CH(F)-aryl, —CH(NH₂)-aryl, —CH(Me)-aryl, or —C(Me)(OH)-aryl.

Most preferred embodiments of the compounds of Formula I or Formula II are those wherein R¹ is —CH(OH)—C≡C—CH₃, —CH(F)—C≡C—CH₃, —CH(NH₂)—C≡C—CH₃, —CH(Me)-C≡C—CH₃, —C(Me)(OH)—C≡C—CH₃, —CH(OH)—C≡C—CH₃, —CH(OH)—C≡C—CF₃, —CH(F)—C≡C—CF₃, —CH(NH₂)—C≡C—CF₃, —CH(Me)-C≡C—CF₃, —C(Me)(OH)—C≡C—CF₃, —CH(OH)—C≡C-cyclopropyl, —CH(F)—C≡C-cyclopropyl, —CH(NH₂)—C≡C-cyclopropyl, —CH(Me)-C≡C-cyclopropyl, —C(Me)(OH)—C≡C-cyclopropyl, —CH(OH)-4-chlorophenyl, —CH(OH)-3,4-dichlorophenyl, —CH(OH)-3,4-difluorophenyl, —CH(OH)-3-fluoro-4-chlorophenyl, —CH(OH)-3-chloro-4-fluorophenyl, —CH(F)-4-chlorophenyl, —CH(F)-3,4-dichlorophenyl, —CH(F)-3,4-difluorophenyl, —CH(F)-3-fluoro-4-chlorophenyl, —CH(F)-3-chloro-4-fluorophenyl, —CH(NH₂)-4-chlorophenyl, —CH(NH₂)-3,4-dichlorophenyl, —CH(NH₂)-3,4-difluorophenyl, —CH(NH₂)-3-fluoro-4-chlorophenyl, —CH(NH₂)-3-chloro-4-fluorophenyl, —CH(Me)-4-chlorophenyl, —CH(Me)-3,4-dichlorophenyl, —CH(Me)-3,4-difluorophenyl, —CH(Me)-3-fluoro-4-chlorophenyl, —CH(Me)-3-chloro-4-fluorophenyl, —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, or —C(Me)(OH)-3-chloro-4-fluorophenyl

Some aspects of the disclosure are directed to compounds of Formula IC and IIC:

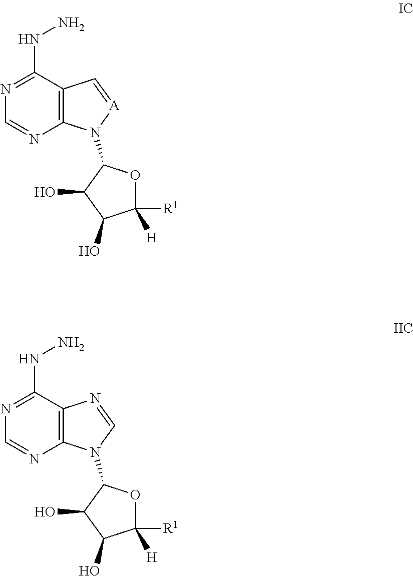

Some aspects of the disclosure are directed to compounds of Formula ID and IID:

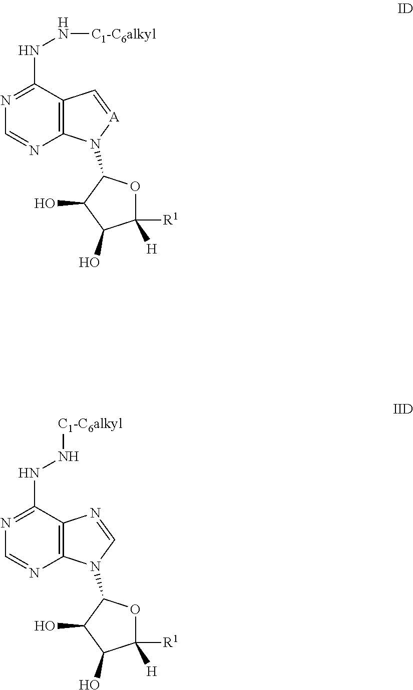

Some aspects of the disclosure are directed to compounds of Formula IE and IIE:

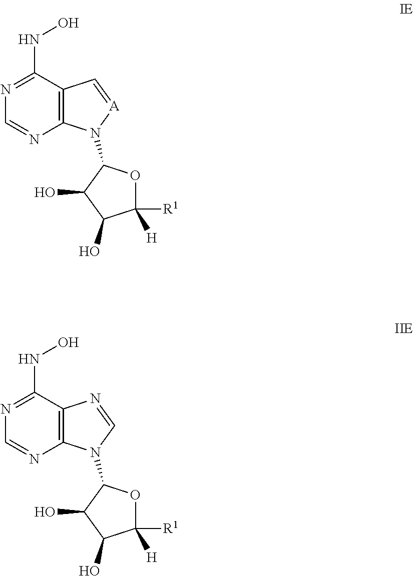

Some aspects of the disclosure are directed to compounds of Formula IF and IIF:

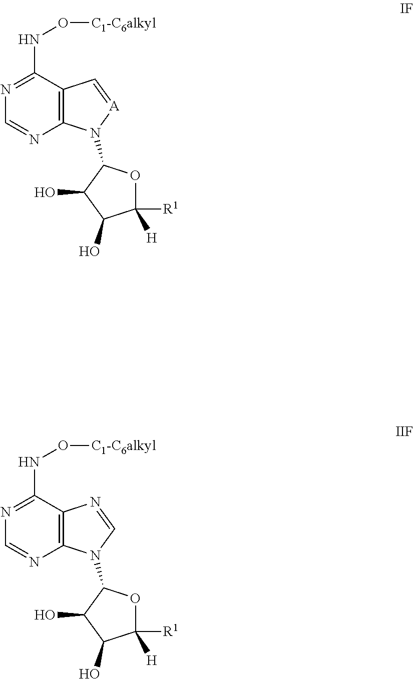

References to Formula I herein include subgenera of Formula I, for example, Formula IA. IA-1, IA-2, IA-3, IA-4, IB, IB-1, IB-2, IB-3, IB-4, IB-5, IB-6, IC, ID, IE, and IF. Similarly, references to Formula II herein include subgenera of Formula II, for example, Formula IIA, IIB, IIC, IID, IIE, IIF, IIG, IIG-1, IIG-2, IIG-3, and IIG-4.

Stereoisomers of compounds of Formula I and Formula II are also contemplated.

Pharmaceutically acceptable salts and solvates of the compounds of Formula I and Formula II are also within the scope of the disclosure.

Isotopic variants of the compounds of Formula I and Formula II are also contemplated by the present disclosure.

In some embodiments, the compounds of Formula I or Formula II are solvates. In preferred embodiments, the compounds of Formula I are ethanolates (i.e., ethanol solvates). In other preferred embodiments, the compounds of Formula II are ethanolates.

In some aspects, the present disclosure is directed to compounds of Formula IB:

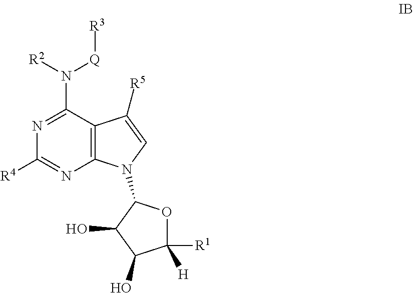

wherein Q is NH or O; R¹ is —C₀-C₆alk-heteroaryl or —C₁-C₆alk-aryl; R² is H; R³ is H, —C₁-C₆alkyl, or —C₁-C₆haloalkyl; R⁴ is H or —C₁-C₆alkyl, and R⁵ is H, halo, or —C₁-C₆alkyl.

In some aspects, the present disclosure is directed to compounds of Formula IB-1

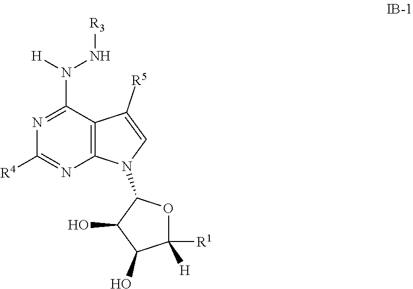

wherein R¹ is —C₀-C₆alk-heteroaryl; R³ is H, —C₁-C₆alkyl, or —C₁-C₆haloalkyl; R⁴ is H or —C₁-C₆alkyl, and R⁵ is H, halo, or —C₁-C₆alkyl.

In other aspects, the disclosure is directed to compounds of Formula IB-1 wherein R¹ is —C₁-C₆alk-aryl; R³ is H, —C₁-C₆alkyl, or —C₁-C₆haloalkyl; R⁴ is H or —C₁-C₆alkyl, and R⁵ is H, halo, or —C₁-C₆alkyl.

Some preferred embodiments are compounds of Formula IB-2

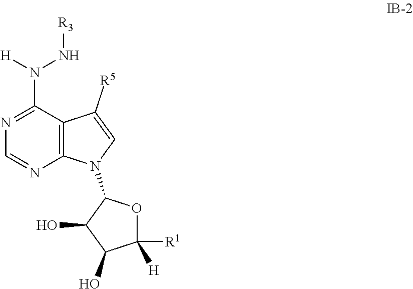

wherein R¹ is 2-(2-amino-3-bromoquinolin-7-yl)ethyl, 2-(2-amino-3-chloroquinolin-7-yl)ethyl, 2-(2-((cyclopropylmethyl)amino)quinolin-7-yl)ethyl, 2-(2-(methylamino)quinolin-7-yl)ethyl, or 2-(2-aminoquinolin-7-yl)ethyl; R³ is H or methyl; and R⁵ is H or F.

Other preferred embodiments are compounds of Formula IB-2 wherein R¹ is —CH(OH)-aryl or —C(Me)(OH)-aryl; H; R³ is H or —C₁-C₆alkyl; and R⁵ is H or halo.

Other preferred embodiments are compounds of Formula IB-2 wherein R¹ is —CH(OH)-4-chlorophenyl, —CH(OH)-3,4-dichlorophenyl, —CH(OH)-3,4-difluorophenyl, —CH(OH)-3-fluoro-4-chlorophenyl, —CH(OH)-3-chloro-4-fluorophenyl, —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, CH(OH)-4-(trifluoromethyl)phenyl, —CH(OH)-3-fluoro-4-(trifluoromethyl)phenyl, —C(CF₃)(OH)-4-chlorophenyl, or —CH(OH)-3-methyl-4-chlorophenyl; R³ is H or methyl; and R⁵ is H or F.

Yet other preferred embodiments are compounds of Formula IB-2 wherein R¹ is —CH(OH)-4-chlorophenyl, —CH(OH)-3,4-dichlorophenyl, or —CH(OH)-4-(trifluoromethyl)phenyl; R³ is H or methyl; and R⁵ is H or F.

Other preferred embodiments are compounds are compounds of Formula IB-2 wherein R¹ is —C(Me)(OH)-3,4-dichlorophenyl or —CH(OH)-3,4-dichlorophenyl, R³ is methyl; and R⁵ is H.

In some aspects, the disclosure is directed to compounds of Formula IB-3

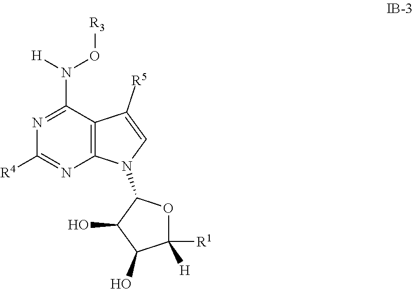

wherein R¹ is —C₀-C₆alk-heteroaryl; R³ is H, —C₁-C₆alkyl, or —C₁-C₆haloalkyl; R⁴ is H or —C₁-C₆alkyl, and R⁵ is H, halo, or —C₁-C₆alkyl.

In other aspects, the disclosure is directed to compounds of Formula IB-3 wherein R¹ is —C₁-C₆alk-aryl; R³ is H, —C₁-C₆alkyl, or —C₁-C₆haloalkyl; R⁴ is H or —C₁-C₆alkyl, and R⁵ is H, halo, or —C₁-C₆alkyl.

Some preferred embodiments are compounds of Formula IB-4

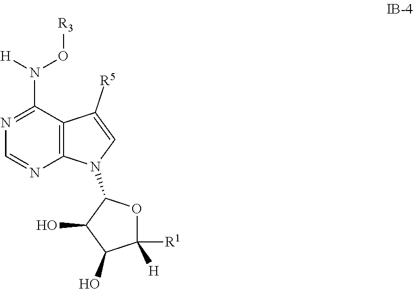

wherein R¹ is 2-(2-amino-3-bromoquinolin-7-yl)ethyl, 2-(2-amino-3-chloroquinolin-7-yl)ethyl, 2-(2-((cyclopropylmethyl)amino)quinolin-7-yl)ethyl, 2-(2-(methylamino)quinolin-7-yl)ethyl, or 2-(2-aminoquinolin-7-yl)ethyl; R³ is H or methyl; and R⁵ is H or F.

Other preferred embodiments are compounds of Formula IB-4 wherein R¹ is —CH(OH)-aryl or —C(Me)(OH)-aryl; R³ is H or —C₁-C₆alkyl; and R⁵ is H or halo.

Other preferred embodiments are compounds of Formula IB-4 wherein R¹ is —CH(OH)-4-chlorophenyl, —CH(OH)-3,4-dichlorophenyl, —CH(OH)-3,4-difluorophenyl, —CH(OH)-3-fluoro-4-chlorophenyl, —CH(OH)-3-chloro-4-fluorophenyl, —CH(OH)-3-methyl-4-(trifluoromethyl)phenyl, —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, CH(OH)-4-(trifluoromethyl)phenyl, —CH(OH)-3-fluoro-4-(trifluoromethyl)phenyl, —C(CF₃)(OH)-4-chlorophenyl, or —CH(OH)-3-methyl-4-chlorophenyl; R³ is H, methyl, or ethyl; and R⁵ is H or F.

Yet other preferred embodiments are compounds of Formula IB-4 wherein R¹ is —CH(OH)-4-chlorophenyl, —CH(OH)-3,4-dichlorophenyl, or —CH(OH)-4-(trifluoromethyl)phenyl; R³ is H or methyl; and R⁵ is H or F.

Other preferred embodiments are compounds of Formula IB-4 wherein R¹ is —C(Me)(OH)-3,4-dichlorophenyl or —CH(OH)-3,4-dichlorophenyl, R³ is methyl; and R⁵ is H.

Other preferred embodiments are compounds of Formula IB-4 wherein R¹ is —CH(OH)-4-chlorophenyl, —CH(OH)-3,4-dichlorophenyl, —CH(OH)-3,4-difluorophenyl, —CH(OH)-3-fluoro-4-chlorophenyl, —CH(OH)-3-chloro-4-fluorophenyl, —CH(OH)-3-methyl-4-(trifluoromethyl)phenyl, —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, CH(OH)-4-(trifluoromethyl)phenyl, —CH(OH)-3-fluoro-4-(trifluoromethyl)phenyl, —C(CF₃)(OH)-4-chlorophenyl, or —CH(OH)-3-methyl-4-chlorophenyl; R³ is —CH₂CF₃ or —CH₂CHF₂; and R⁵ is H or F.

Other preferred embodiments are compounds of Formula IB-4 wherein R¹ is —CH(OH)-4-chlorophenyl, —CH(OH)-3,4-dichlorophenyl, —CH(OH)-3,4-difluorophenyl, —CH(OH)-3-fluoro-4-chlorophenyl, —CH(OH)-3-chloro-4-fluorophenyl, —CH(OH)-3-methyl-4-(trifluoromethyl)phenyl, —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, CH(OH)-4-(trifluoromethyl)phenyl, —CH(OH)-3-fluoro-4-(trifluoromethyl)phenyl, —C(CF₃)(OH)-4-chlorophenyl, or —CH(OH)-3-methyl-4-chlorophenyl; R³ is H, methyl, ethyl. —CH₂CF₃, or —CH₂CHF₂; and R⁵ is hydroxymethyl (i.e., CH₂OH), hydroxyethyl (i.e., —CH₂CH₂OH), or ethynyl.

Some preferred embodiments are compounds of Formula IB-5

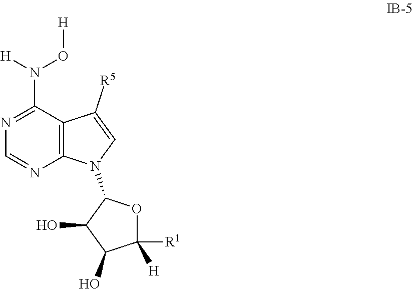

wherein R¹ is R¹ is —CH(OH)-4-chlorophenyl, —CH(OH)-3,4-dichlorophenyl, —CH(OH)-3,4-difluorophenyl, —CH(OH)-3-fluoro-4-chlorophenyl, —CH(OH)-3-chloro-4-fluorophenyl, —C(Me)(OH)-4-chlorophenyl, or —C(Me)(OH)-3,4-dichlorophenyl, and R⁵ is H or F.

Other preferred embodiments are compounds of Formula IB-6

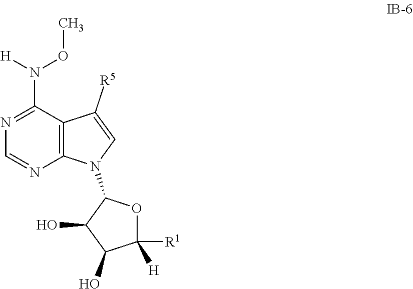

wherein R¹—CH(OH)-3,4-dichlorophenyl, —CH(OH)-3-chloro-4-fluorophenyl, —C(Me)(OH)-3,4-dichlorophenyl, —CH(OH)-3-fluoro-4-(trifluoromethyl)phenyl, —CH(OH)-3-methyl-4-(trifluoromethyl)phenyl, or —CH(OH)-3-methyl-4-chlorophenyl; and R⁵ is H.

In other aspects, the disclosure is directed to compounds of Formula II-G:

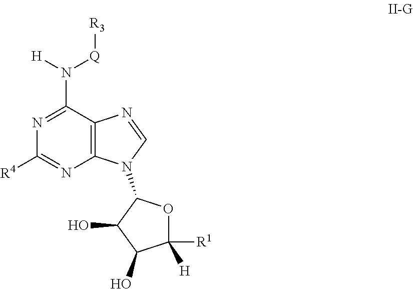

wherein Q is NH or O, R¹ is —C₀-C₆alk-heteroaryl or —C₁-C₆alk-aryl; R³ is H, —C₁-C₆alkyl, or —C₁-C₆haloalkyl; and R⁴ is H or —C₁-C₆alkyl.

Some preferred embodiments are compounds of Formula II-G-1

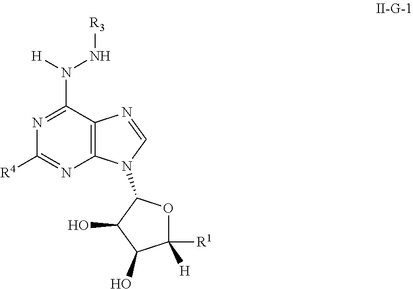

wherein R¹ is —C₀-C₆alk-heteroaryl; R³ is H, —C₁-C₆alkyl, or —C₁-C₆haloalkyl; and R⁴ is H or —C₁-C₆alkyl.

Other preferred embodiments are compounds of Formula II-G-1 wherein R¹ is 2-(2-amino-3-bromoquinolin-7-yl)ethyl, 2-(2-amino-3-chloroquinolin-7-yl)ethyl, 2-(2-((cyclopropylmethyl)amino)quinolin-7-yl)ethyl, 2-(2-(methylamino)quinolin-7-yl)ethyl, or 2-(2-aminoquinolin-7-yl)ethyl; R³ is H or methyl; and R⁴ is H.

Other preferred embodiments are compounds of Formula II-G-1 wherein R¹ is —C₁-C₆alk-aryl; R³ is H, —C₁-C₆alkyl, or —C₁-C₆haloalkyl; and R⁴ is H or —C₁-C₆alkyl.

Other preferred embodiments are compounds of Formula II-G-1 wherein R¹ is —CH(OH)-aryl or —C(Me)(OH)-aryl; R³ is H, —C₁-C₆alkyl, or —C₁-C₆haloalkyl; and R⁴ is H or —C₁-C₆alkyl.

Other preferred embodiments are compounds of Formula II-G-1 wherein R¹ is —CH(OH)-4-chlorophenyl, —CH(OH)-3,4-dichlorophenyl, —CH(OH)-3,4-difluorophenyl, —CH(OH)-3-fluoro-4-chlorophenyl, —CH(OH)-3-chloro-4-fluorophenyl, —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, CH(OH)-4-(trifluoromethyl)phenyl, —CH(OH)-3-fluoro-4-(trifluoromethyl)phenyl, —C(CF₃)(OH)-4-chlorophenyl, or —CH(OH)-3-methyl-4-chlorophenyl; R³ is H or methyl; and R⁴ is H or methyl.

In some aspects, the present disclosure is directed to compounds of Formula II-G-2

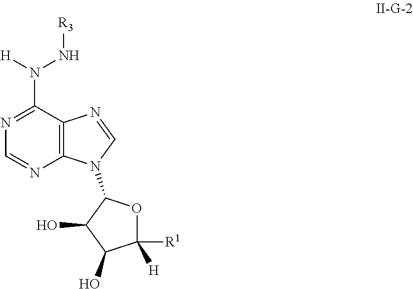

wherein R¹ is 2-(2-amino-3-bromoquinolin-7-yl)ethyl, 2-(2-amino-3-chloroquinolin-7-yl)ethyl, 2-(2-((cyclopropylmethyl)amino)quinolin-7-yl)ethyl, 2-(2-(methylamino)quinolin-7-yl)ethyl, or 2-(2-aminoquinolin-7-yl)ethyl; and R³ is H or methyl.

In other aspects, the present disclosure is directed to compounds of Formula II-G-2 wherein R¹ is —CH(OH)-4-chlorophenyl, and R³ is H or methyl.

In some aspects, the present disclosure is directed to compounds of Formula II-G-3

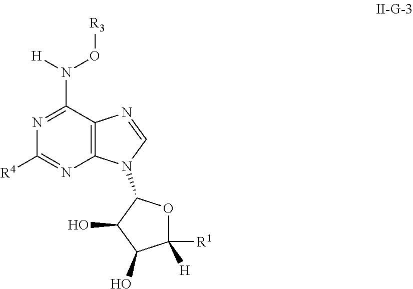

wherein R¹ is —C₀-C₆alk-heteroaryl; R³ is H, —C₁-C₆alkyl, or —C₁-C₆haloalkyl; and R⁴ is H or -C₁-C₆alkyl.

Some preferred embodiments are compounds of Formula II-G-3 wherein R¹ is 2-(2-amino-3-bromoquinolin-7-yl)ethyl, 2-(2-amino-3-chloroquinolin-7-yl)ethyl, 2-(2-((cyclopropylmethyl)amino)quinolin-7-yl)ethyl, 2-(2-(methylamino)quinolin-7-yl)ethyl, or 2-(2-aminoquinolin-7-yl)ethyl; R³ is H or methyl; and R⁴ is H.

Other preferred embodiments are compounds of Formula II-G-3 wherein R¹ is —C₁-C₆alk-aryl; R³ is H, —C₁-C₆alkyl, or —C₁-C₆haloalkyl; and R⁴ is H or —C₁-C₆alkyl.

Other preferred embodiments are compounds of Formula II-G-3 wherein R¹ is —CH(OH)-aryl or —C(Me)(OH)-aryl; R³ is H, —C₁-C₆alkyl, or —C₁-C₆haloalkyl; and R⁴ is H or —C₁-C₆alkyl.

Other preferred embodiments are compounds of Formula II-G-3 wherein R¹ is —CH(OH)-4-chlorophenyl, —CH(OH)-3,4-dichlorophenyl, —CH(OH)-3,4-difluorophenyl, —CH(OH)-3-fluoro-4-chlorophenyl, —CH(OH)-3-chloro-4-fluorophenyl, —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, CH(OH)-4-(trifluoromethyl)phenyl, —CH(OH)-3-fluoro-4-(trifluoromethyl)phenyl, —C(CF₃)(OH)-4-chlorophenyl, or —CH(OH)-3-methyl-4-chlorophenyl; R³ is H, methyl, or ethyl; and R⁴ is H or methyl.

In some aspects, the present disclosure is directed to compounds of Formula II-G-4

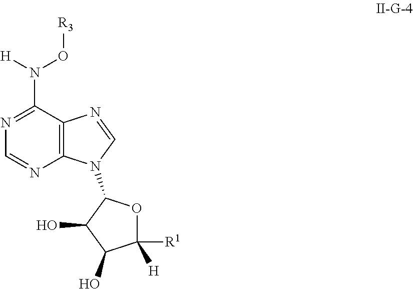

wherein R¹ is 2-(2-amino-3-bromoquinolin-7-yl)ethyl, 2-(2-amino-3-chloroquinolin-7-yl)ethyl, 2-(2-((cyclopropylmethyl)amino)quinolin-7-yl)ethyl, 2-(2-(methylamino)quinolin-7-yl)ethyl, or 2-(2-aminoquinolin-7-yl)ethyl; and R³ is H or methyl.

In other aspects, the present disclosure is directed to compounds of Formula II-G-4 wherein R¹ is —CH(OH)-4-chlorophenyl, —CH(OH)-3,4-dichlorophenyl, —CH(OH)-3,4-difluorophenyl, —CH(OH)-3-fluoro-4-chlorophenyl, —CH(OH)-3-chloro-4-fluorophenyl, —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, CH(OH)-4-(trifluoromethyl)phenyl, —CH(OH)-3-fluoro-4-(trifluoromethyl)phenyl, —C(CF₃)(OH)-4-chlorophenyl, or —CH(OH)-3-methyl-4-chlorophenyl; and R³ is H or methyl.

In yet other aspects, the present disclosure is directed to compounds of Formula II-G-4 wherein R¹ is —CH(OH)-4-chlorophenyl, and R³ is H or methyl.

In some aspects, the present disclosure is directed to compounds of Formula IA:

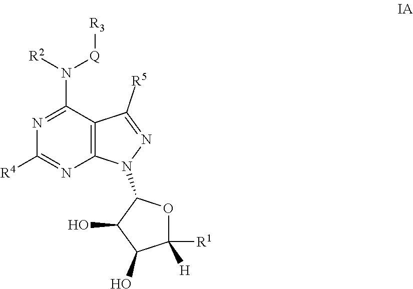

wherein Q is NH or O; R¹ is —C₀-C₆alk-heteroaryl or —C₁-C₆alk-aryl; R² is H; R³ is H, —C₁-C₆alkyl, or —C₁-C₆haloalkyl; R⁴ is H or —C₁-C₆alkyl, and R⁵ is H, halo, or —C₁-C₆alkyl.

In some aspects, the present disclosure is directed to compounds of Formula IA-1

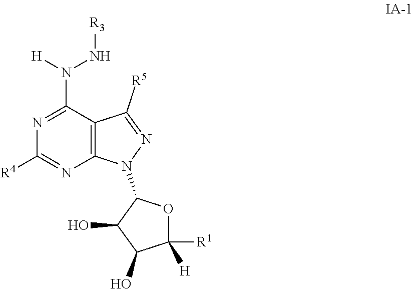

wherein R¹ is —C₀-C₆alk-heteroaryl; R³ is H, —C₁-C₆alkyl, or —C₁-C₆haloalkyl; R⁴ is H or —C₁-C₆alkyl, and R⁵ is H, halo, or —C₁-C₆alkyl.

In other aspects, the disclosure is directed to compounds of Formula IA-1 wherein R¹ is —C₁-C₆alk-aryl; R³ is H, —C₁-C₆alkyl, or —C₁-C₆haloalkyl; R⁴ is H or —C₁-C₆alkyl, and R⁵ is H, halo, or —C₁-C₆alkyl.

Some preferred embodiments are compounds of Formula IA-2

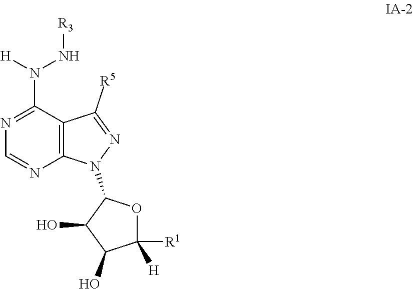

wherein R¹ is 2-(2-amino-3-bromoquinolin-7-yl)ethyl, 2-(2-amino-3-chloroquinolin-7-yl)ethyl, 2-(2-((cyclopropylmethyl)amino)quinolin-7-yl)ethyl, 2-(2-(methylamino)quinolin-7-yl)ethyl, or 2-(2-aminoquinolin-7-yl)ethyl; R³ is H or methyl; and R⁵ is H or F.

Other preferred embodiments are compounds of Formula IA-2 wherein R¹ is —CH(OH)-aryl or —C(Me)(OH)-aryl; R³ is H or —C₁-C₆alkyl; and R⁵ is H or halo.

Other preferred embodiments are compounds of Formula IA-2 wherein R¹ is —CH(OH)-4-chlorophenyl, —CH(OH)-3,4-dichlorophenyl, —CH(OH)-3,4-difluorophenyl, —CH(OH)-3-fluoro-4-chlorophenyl, —CH(OH)-3-chloro-4-fluorophenyl, —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, CH(OH)-4-(trifluoromethyl)phenyl, —CH(OH)-3-fluoro-4-(trifluoromethyl)phenyl, —C(CF₃)(OH)-4-chlorophenyl, or —CH(OH)-3-methyl-4-chlorophenyl; R³ is H or methyl; and R⁵ is H or F.

Yet other preferred embodiments are compounds of Formula IA-2 wherein R¹ is —CH(OH)-4-chlorophenyl, —CH(OH)-3,4-dichlorophenyl, or —CH(OH)-4-(trifluoromethyl)phenyl; R³ is H or methyl; and R⁵ is H or F.

In some aspects, the disclosure is directed to compounds of Formula IA-3

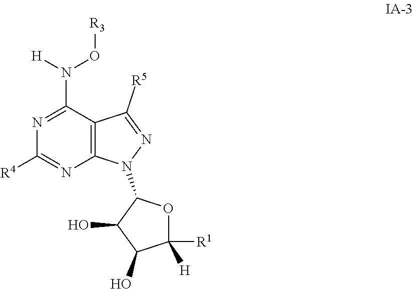

wherein R¹ is —C₀-C₆alk-heteroaryl; R³ is H, —C₁-C₆alkyl, or —C₁-C₆haloalkyl; R⁴ is H or —C₁-C₆alkyl, and R⁵ is H, halo, or —C₁-C₆alkyl.

In other aspects, the disclosure is directed to compounds of Formula IA-3 wherein R¹ is —C₁-C₆alk-aryl; R³ is H, —C₁-C₆alkyl, or —C₁-C₆haloalkyl; R⁴ is H or —C₁-C₆alkyl, and R⁵ is H, halo, or —C₁-C₆alkyl.

Some preferred embodiments are compounds of Formula IA-4

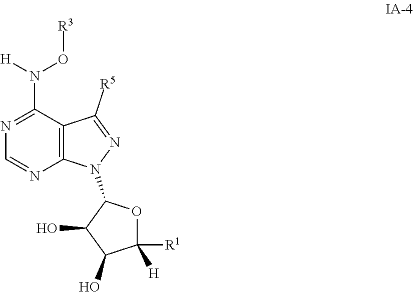

wherein R¹ is 2-(2-amino-3-bromoquinolin-7-yl)ethyl, 2-(2-amino-3-chloroquinolin-7-yl)ethyl, 2-(2-((cyclopropylmethyl)amino)quinolin-7-yl)ethyl, 2-(2-(methylamino)quinolin-7-yl)ethyl, or 2-(2-aminoquinolin-7-yl)ethyl; R³ is H or methyl; and R⁵ is H or F.

Other preferred embodiments are compounds of Formula IA-4 wherein R¹ is —CH(OH)-aryl or —C(Me)(OH)-aryl; H; R³ is H or —C₁-C₆alkyl; and R⁵ is H or halo.

Other preferred embodiments are compounds of Formula IA-4 wherein R¹ is —CH(OH)-4-chlorophenyl, —CH(OH)-3,4-dichlorophenyl, —CH(OH)-3,4-difluorophenyl, —CH(OH)-3-fluoro-4-chlorophenyl, —CH(OH)-3-chloro-4-fluorophenyl, —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, CH(OH)-4-(trifluoromethyl)phenyl, —CH(OH)-3-fluoro-4-(trifluoromethyl)phenyl, —C(CF₃)(OH)-4-chlorophenyl, or —CH(OH)-3-methyl-4-chlorophenyl; R³ is H or methyl; and R⁵ is H or F.

Yet other preferred embodiments are compounds of Formula IA-4 wherein R¹ is —CH(OH)-4-chlorophenyl, —CH(OH)-3,4-dichlorophenyl, or —CH(OH)-4-(trifluoromethyl)phenyl; R³ is H or methyl; and R⁵ is H or F.

In some aspects, the present disclosure is directed to a crystalline form of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol (Example 92A).

A crystal form may be referred to herein as being characterized by graphical data “as shown in” or “as characterized by” a Figure. Such data include, for example, powder X-ray diffractograms (XRPD), Differential Scanning Calorimetry (DSC) thermograms, thermogravimetric analysis (TGA) profiles, and differential vapor sorption profiles (DVS). As is well-known in the art, the graphical data potentially provides additional technical information to further define the respective solid state form which can not necessarily be described by reference to numerical values or peak positions alone. Thus, the term “substantially as shown in” when referring to graphical data in a Figure herein means a pattern that is not necessarily identical to those depicted herein, but that falls within the limits of experimental error or deviations, when considered by one of ordinary skill in the art. The skilled person would readily be able to compare the graphical data in the Figures herein with graphical data generated for an unknown crystal form and confirm whether the two sets of graphical data are characterizing the same crystal form or two different crystal forms.

A solid, crystalline form may be referred to herein as “polymorphically pure” or as “substantially free of any other form.” As used herein in this context, the expression “substantially free of any other forms” will be understood to mean that the solid form contains about 20% or less, about 10% or less, about 5% or less, about 2% or less, about 1% or less, or 0% of any other forms of the subject compound as measured, for example, by XRPD. Thus, a solid form of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol described herein as substantially free of any other solid forms would be understood to contain greater than about 80% (w/w), greater than about 90% (w/w), greater than about 95% (w/w), greater than about 98% (w/w), greater than about 99% (w/w), or about 100% of the subject solid form of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol. Accordingly, in some embodiments of the disclosure, the described solid form of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol may contain from about 1% to about 20% (w/w), from about 5% to about 20% (w/w), or from about 5% to about 10% (w/w) of one or more other solid forms of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol.

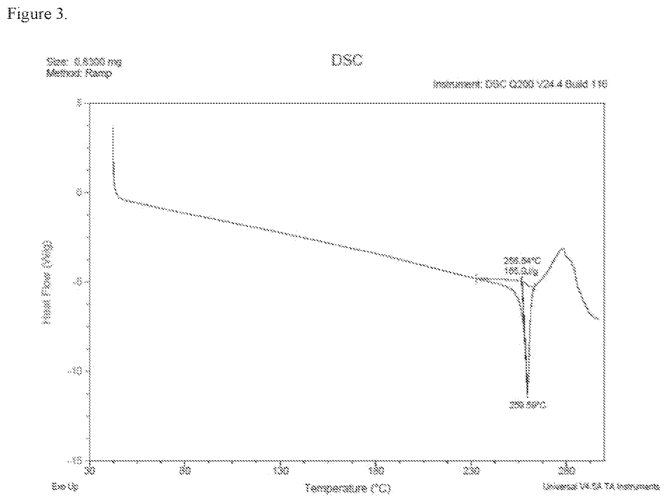

In some aspects of the present disclosure, the solid form of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol is crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol. In other aspects, the solid form is crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol substantially free of any other solid form of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol. Crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol exhibits an XRPD substantially as shown in FIG. 2.

The XRPD of crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol shown in FIG. 2 comprises reflection angles (degrees 2-theta±0.2 degrees 2-theta), line spacings (d Values), and relative intensities as shown in Table 6 (below).

In some embodiments of the present disclosure, crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol is characterized by an XRPD pattern comprising a peak at one of the angles listed in Table 6. In other aspects, crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol is characterized by an XRPD pattern comprising more than one peak at one of the angles listed in Table 6 below. In other aspects, crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol is characterized by an XRPD pattern comprising two peaks selected from the angles listed in Table 6 below. In other aspects, crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol is characterized by an XRPD pattern comprising three peaks selected from the angles listed in Table 6 below. In other aspects, crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol is characterized by an XRPD pattern comprising four peaks selected from the angles listed in Table 6 below. In other aspects, crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol is characterized by an XRPD pattern comprising five peaks selected from the angles listed in Table 6 below. In other aspects, crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol is characterized by an XRPD pattern comprising six peaks selected from the angles listed in Table 6 below. In other aspects, crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol is characterized by an XRPD pattern comprising seven peaks selected from the angles listed in Table 6 below. In other aspects, crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol is characterized by an XRPD pattern comprising eight peaks selected from the angles listed in Table 6 below. In other aspects, crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol is characterized by an XRPD pattern comprising nine peaks selected from the angles listed in Table 6 below. In other aspects, crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol is characterized by an XRPD pattern comprising ten peaks selected from the angles listed in Table 6 below. In other aspects, crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol is characterized by an XRPD pattern comprising more than ten peaks selected from the angles listed in Table 6 below.

Crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol can be characterized by a DSC thermogram substantially as shown in FIG. 3. As FIG. 3 shows, crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol produced an endothermic peak at about 259.59° C. In some embodiments of the present disclosure, crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol is characterized by a DSC thermogram comprising an endothermic peak at about 259° C.

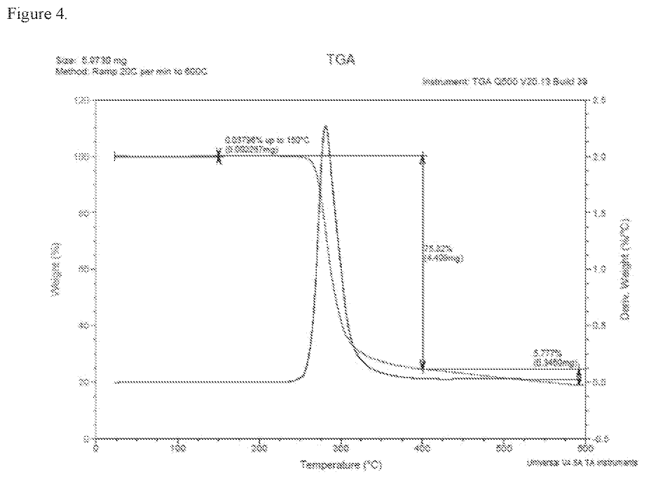

Crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol can be characterized by a TGA profile substantially as shown in FIG. 4.

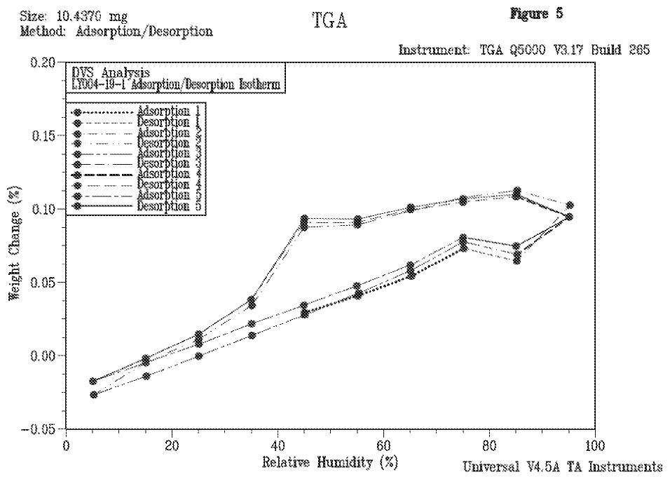

Crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol can be characterized by a DVS profile substantially as shown in FIG. 5.

In some embodiments of the present disclosure, crystalline Form I of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol is characterized by an XRPD pattern comprising peaks at three or more of the angles listed in Table 6 below, and a DSC thermogram comprising an endothermic peak at about 259° C.

The disclosure is also directed to compounds of Formula III or Formula IV. In some aspects, the disclosure is directed to compounds of Formula III:

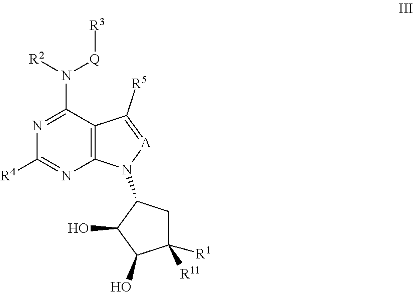

In other aspects, the disclosure is directed to compounds of Formula IV:

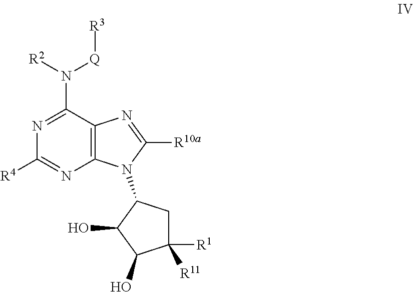

According to the disclosure, A in Formula III is CH, CR¹⁰, or N. In some aspects, A is N and the compounds of Formula III are of Formula IIIA:

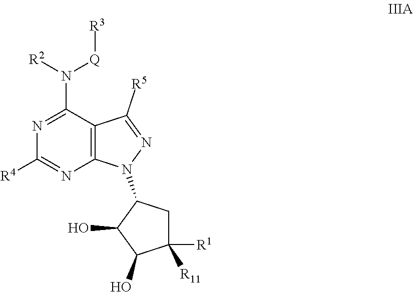

In other aspects, A is CH and the compounds of Formula III are of Formula IIIB:

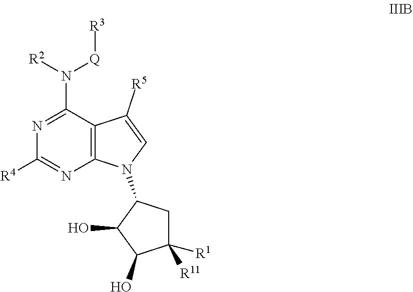

In yet other aspects, A is CR¹⁰ and the compounds of Formula III are of Formula IIIC:

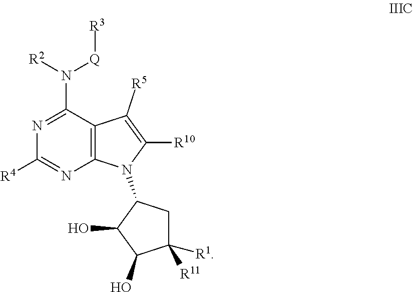

According to the disclosure, Q in Formula III or Formula IV is NH, NR⁶, or O. In some embodiments, Q is NH. In other embodiments, Q is O. In yet other embodiments, Q is NR⁶.

According to the disclosure, R¹ in Formula III or Formula IV is —C₀-C₆alk-C₃-C₆cycloalkyl, —C₀-C₆alk-C₃-C₆halocycloalkyl, —C₂-C₆alkenyl, —C₂-C₆haloalkenyl, —C₀-C₆alk-C₁-C₆alkyl, —C₀-C₆alk-C₁-C₆haloalkyl, —C₀-C₆alk-C≡CH, —C₀-C₆alk-C≡C—C₁-C₆alkyl, —C₀-C₆alk-C≡C—C₁-C₆haloalkyl, —C₀-C₆alk-C≡C—C₃-C₆cycloalkyl, —C₁-C₆alk-aryl, —C₁-C₆alk-S—C₁-C₆alkyl, —C₁-C₆alk-S—C₁-C₆haloalkyl, —C₁-C₆alk-S—C₃-C₆cycloalkyl; —C₁-C₆alk-S—C₃-C₆halocycloalkyl; —C₁-C₆alk-O—C₁-C₆alkyl, —C₁-C₆alk-O—C₃-C₆cycloalkyl, —C₁-C₆alk-S—CH₂-aryl, —C₁-C₆alk-C(O)NH-aryl, —C₀-C₆alk-S-aryl, —C₀-C₆alk-S(O)aryl, —C₀-C₆alk-S(O)₂aryl, —C₀-C₆alk-Oaryl, —C₀-C₆alk-heteroaryl, —C₁-C₆alk-O-heteroaryl, —C₁-C₆alk-S-heteroaryl, or —C₁-C₆alk-NH-heteroaryl.

In some aspects, R¹ in Formula III or Formula IV is —C₀-C₆alk-C₁-C₆alkyl, —C₀-C₆alk-C₁-C₆haloalkyl, —C₀-C₆alk-C≡CH, —C₀-C₆alk-C≡C—C₁-C₆alkyl, —C₀-C₆alk-C≡C—C₁-C₆haloalkyl, —C₀-C₆alk-C≡C—C₃-C₆cycloalkyl, —C₁-C₆alk-aryl, —C₀-C₆alk-S-aryl, —C₀-C₆alk-S(O)aryl, —C₀-C₆alk-S(O)₂aryl, or —C₀-C₆alk-Oaryl.

In other aspects, R¹ in Formula III or Formula IV is —C₀-C₆alk-C₃-C₆cycloalkyl, for example, —C₀alk-C₃cycloalkyl, —C₁alk-C₃cycloalkyl, —C₂alk-C₃cycloalkyl, —C₃alk-C₃cycloalkyl, —C₄alk-C₃cycloalkyl, —C₅alk-C₃cycloalkyl, —C₆alk-C₃cycloalkyl, —C₀alk-C₄cycloalkyl, —C₁alk-C₄cycloalkyl, —C₂alk-C₄cycloalkyl, —C₃alk-C₄cycloalkyl, —C₄alk-C₄cycloalkyl, —C₅alk-C₄cycloalkyl, —C₆alk-C₄cycloalkyl, —C₀alk-C₅cycloalkyl, —C₁alk-C₅cycloalkyl, —C₂alk-C₅cycloalkyl, —C₃alk-C₅cycloalkyl, —C₄alk-C₅cycloalkyl, —C₅alk-C₅cycloalkyl, —C₆alk-C₅cycloalkyl, —C₀alk-C₆cycloalkyl, —C₁alk-C₆cycloalkyl, —C₂alk-C₆cycloalkyl, —C₃alk-C₆cycloalkyl, —C₄alk-C₆cycloalkyl, —C₅alk-C₆cycloalkyl, or —C₆alk-C₆cycloalkyl. Thus, in some aspects, R¹ is —CH₂-cyclopropyl.

In some aspects, R¹ in Formula III or Formula IV is —C₀-C₆alk-C₃-C₆halocycloalkyl, for example, —C₀alk-C₃halocycloalkyl, —C₁alk-C₃halocycloalkyl, —C₂alk-C₃halocycloalkyl, —C₃alk-C₃halocycloalkyl, —C₄alk-C₃halocycloalkyl, —C₅alk-C₃halocycloalkyl, —C₆alk-C₃halocycloalkyl, —C₀alk-C₄halocycloalkyl, —C₁alk-C₄halocycloalkyl, —C₂alk-C₄halocycloalkyl, —C₃alk-C₄halocycloalkyl, —C₄alk-C₄halocycloalkyl, —C₅alk-C₄halocycloalkyl, —C₆alk-C₄halocycloalkyl, —C₀alk-C₅halocycloalkyl, —C₁alk-C₅halocycloalkyl, —C₂alk-C₅halocycloalkyl, —C₃alk-C₅halocycloalkyl, —C₄alk-C₅halocycloalkyl, —C₅alk-C₅halocycloalkyl, —C₆alk-C₅halocycloalkyl, —C₀alk-C₆halocycloalkyl, —C₁alk-C₆halocycloalkyl, —C₂alk-C₆halocycloalkyl, —C₃alk-C₆halocycloalkyl, —C₄alk-C₆halocycloalkyl, —C₅alk-C₆halocycloalkyl, or —C₆alk-C₆halocycloalkyl.

In some aspects, R¹ in Formula III or Formula IV is —C₂-C₆alkenyl, for example, vinyl, allyl, and the like.

In some aspects, R¹ in Formula III or Formula IV is —C₂-C₆haloalkenyl, for example, —C(F)═CHMe, —C(F)═CH₂, and the like.

In some aspects, R¹ in Formula III or Formula IV is —C₀-C₆alk-C₁-C₆alkyl, for example, —C₀alk-C₁alkyl, —C₁alk-C₁alkyl, —C₂alk-C₁alkyl, —C₃alk-C₁alkyl, —C₄alk-C₁alkyl, —C₅alk-C₁alkyl, —C₀alk-C₁alkyl, —C₀alk-C₂alkyl, —C₁alk-C₂alkyl, —C₂alk-C₂alkyl, —C₃alk-C₂alkyl, —C₄alk-C₂alkyl, —C₅alk-C₂alkyl, —C₆alk-C₂alkyl, —C₀alk-C₃alkyl, —C₁alk-C₃alkyl, —C₂alk-C₃alkyl, —C₃alk-C₃alkyl, —C₄alk-C₃alkyl, —C₅alk-C₃alkyl, —C₆alk-C₃alkyl, —C₀alk-C₄alkyl, —C₁alk-C₄alkyl, —C₂alk-C₄alkyl, —C₃alk-C₄alkyl, —C₄alk-C₄alkyl, —C₅alk-C₄alkyl, —C₆alk-C₄alkyl, —C₀alk-C₅alkyl, —C₁alk-C₅alkyl, —C₂alk-C₅alkyl, —C₃alk-C₅alkyl, —C₄alk-C₅alkyl, —C₅alk-C₅alkyl, —C₆alk-C₅alkyl, —C₀alk-C₆alkyl, —C₁alk-C₆alkyl, —C₂alk-C₆alkyl, —C₃alk-C₆alkyl, —C₄alk-C₆alkyl, —C₅alk-C₆alkyl, —C₆alk-C₆alkyl, methyl, ethyl, propyl, isopropyl, butyl, isobutyl, s-butyl, t-butyl, pentyl, —CH(OH)—C₁-C₆alkyl, —CH(F)—C₁-C₆alkyl, —CH(NH₂)—C₁-C₆alkyl, —CH(Me)-C₁-C₆alkyl, —C(Me)(OH)—C₁-C₆alkyl, and the like.

In other aspects, R¹ in Formula III or Formula IV is —C₀-C₆alk-C₁-C₆haloalkyl, for example, —C₀alk-C₁haloalkyl, —C₁alk-C₁haloalkyl, —C₂alk-C₁haloalkyl, —C₃alk-C₁haloalkyl, —C₄alk-C₁haloalkyl, —C₅alk-C₁haloalkyl, —C₀alk-C₁haloalkyl, —C₀alk-C₂haloalkyl, —C₁alk-C₂haloalkyl, —C₂alk-C₂haloalkyl, —C₃alk-C₂haloalkyl, —C₄alk-C₂haloalkyl, —C₅alk-C₂haloalkyl, —C₆alk-C₂haloalkyl, —C₀alk-C₃haloalkyl, —C₁alk-C₃haloalkyl, —C₂alk-C₃haloalkyl, —C₃alk-C₃haloalkyl, —C₄alk-C₃haloalkyl, —C₅alk-C₃haloalkyl, —C₆alk-C₃haloalkyl, —C₀alk-C₄haloalkyl, —C₁alk-C₄haloalkyl, —C₂alk-C₄haloalkyl, —C₃alk-C₄haloalkyl, —C₄alk-C₄haloalkyl, —C₅alk-C₄haloalkyl, —C₆alk-C₄haloalkyl, —C₀alk-C₅haloalkyl, —C₁alk-C₅haloalkyl, —C₂alk-C₅haloalkyl, —C₃alk-C₅haloalkyl, —C₄alk-C₅haloalkyl, —C₅alk-C₅haloalkyl, —C₆alk-C₅haloalkyl, —C₀alk-C₆haloalkyl, —C₁alk-C₆haloalkyl, —C₂alk-C₆haloalkyl, —C₃alk-C₆haloalkyl, —C₄alk-C₆haloalkyl, —C₅alk-C₆haloalkyl, —C₆alk-C₆haloalkyl, fluoromethyl, fluoroethyl, fluoropropyl, fluorobutyl, fluoropentyl, chloromethyl, chloroethyl, chloropropyl, chlorobutyl, chloropentyl, bromomethyl, bromoethyl, bromopropyl, bromobutyl, bromopentyl, iodomethyl, iodoethyl, iodopropyl, iodobutyl, iodopentyl, —CH(OH)—C₁-C₆ haloalkyl, —CH(F)—C₁-C₆ haloalkyl, —CH(NH₂)—C₁-C₆ haloalkyl, —CH(Me)-C₁-C₆ haloalkyl, —C(Me)(OH)—C₁-C₆ haloalkyl, and the like.

In some aspects, R¹ in Formula III or Formula IV is —C₀-C₆alk-C≡CH, for example, —C₀alk-C≡CH, —C₁alk-C≡CH, —C₂alk-C≡CH, —C₃alk-C≡CH, —C₄alk-C≡CH, —C₅alk-C≡CH, —C₆alk-C—CH, ethynyl, propargyl, —CH(OH)—C≡CH, —CH(F)—C≡CH, —CH(NH₂)—C≡CH, —CH(Me)-C≡CH, —C(Me)(OH)—C≡CH, and the like.

In some aspects, R¹ in Formula III or Formula IV is —C₀-C₆alk-C≡C—C₁-C₆alkyl, for example, —C₀alk-C≡C—C₁alkyl, —C₁alk-C≡C—C₁alkyl, —C₂alk-C≡C—C₁alkyl, —C₃alk-C≡C—C₁alkyl, —C₄alk-C≡C—C₁alkyl, —C₅alk-C≡C—C₁alkyl, —C₀alk-C≡C—C₁alkyl, —C₀alk-C≡C—C₂alkyl, —C₁alk-C≡C—C₂alkyl, —C₂alk-C≡C—C₂alkyl, —C₃alk-C≡C—C₂alkyl, —C₄alk-C≡C—C₂alkyl, —C₅alk-C≡C—C₂alkyl, —C₆alk-C≡C—C₂alkyl, —C₀alk-C≡C—C₃alkyl, —C₁alk-C≡C—C₃alkyl, —C₂alk-C≡C—C₃alkyl, —C₃alk-C≡C—C₃alkyl, —C₄alk-C≡C—C₃alkyl, —C₅alk-C≡C—C₃alkyl, —C₆alk-C≡C—C₃alkyl, —C₀alk-C≡C—C₄alkyl, —C₁alk-C≡C—C₄alkyl, —C₂alk-C≡C—C₄alkyl, —C₃alk-C≡C—C₄alkyl, —C₄alk-C≡C—C₄alkyl, —C₅alk-C≡C—C₄alkyl, —C₆alk-C≡C—C₄alkyl, —C₀alk-C≡C—C₅alkyl, —C₁alk-C≡C—C₅alkyl, —C₂alk-C≡C—C₅alkyl, —C₃alk-C≡C—C₅alkyl, —C₄alk-C≡C—C₅alkyl, —C₅alk-C≡C—C₅alkyl, —C₆alk-C≡C—C₅alkyl, —C₀alk-C≡C—C₆alkyl, —C₁alk-C≡C—C₆alkyl, —C₂alk-C≡C—C₆alkyl, —C₃alk-C≡C—C₆alkyl, —C₄alk-C≡C—C₆alkyl, —C₅alk-C≡C—C₆alkyl, —C₀alk-C≡C—C₆alkyl, propynyl, butynyl, —CH(OH)—C≡C—C₁-C₆alkyl, —CH(F)—C≡C—C₁-C₆alkyl, —CH(NH₂)—C≡C—C₁-C₆alkyl, —CH(Me)-C≡C—C₁-C₆alkyl, —C(Me)(OH)—C≡C—C₁-C₆alkyl, and the like. In some embodiments wherein —C₀-C₆alk-C≡C—C₁-C₆alkyl is —C₀-C₆alk-C≡C—CH₃, R¹ is —CH(OH)—C≡C—CH₃, —CH(F)—C≡C—CH₃, —CH(NH₂)—C≡C—CH₃, —CH(Me)-C≡C—CH₃, or —C(Me)(OH)—C≡C—CH₃. Thus, in some embodiments, R¹ is —CH(OH)—C≡C—CH₃.

In some aspects, R¹ in Formula III or Formula IV is —C₀-C₆alk-C≡C—C₁-C₆haloalkyl, for example, —C₀alk-C≡C—C₁haloalkyl, —C₁alk-C≡C—C₁haloalkyl, —C₂alk-C≡C—C₁haloalkyl, —C₃alk-C≡C—C₁haloalkyl, —C₄alk-C≡C—C₁haloalkyl, —C₅alk-C≡C—C₁haloalkyl, —C₆alk-C≡C—C₁haloalkyl, —C₀alk-C≡C—C₂haloalkyl, —C₁alk-C≡C—C₂haloalkyl, —C₂alk-C≡C—C₂haloalkyl, —C₃alk-C≡C—C₂haloalkyl, —C₄alk-C≡C—C₂haloalkyl, —C₅alk-C≡C—C₂haloalkyl, —C₆alk-C≡C—C₂haloalkyl, —C₀alk-C≡C—C₃haloalkyl, —C₁alk-C≡C—C₃haloalkyl, —C₂alk-C≡C—C₃haloalkyl, —C₃alk-C≡C—C₃haloalkyl, —C₄alk-C≡C—C₃haloalkyl, —C₅alk-C≡C—C₃haloalkyl, —C₆alk-C≡C—C₃haloalkyl, —C₀alk-C≡C—C₄haloalkyl, —C₁alk-C≡C—C₄haloalkyl, —C₂alk-C≡C—C₄haloalkyl, —C₃alk-C≡C—C₄haloalkyl, —C₄alk-C≡C—C₄haloalkyl, —C₅alk-C≡C—C₄haloalkyl, —C₆alk-C≡C—C₄haloalkyl, —C₀alk-C≡C—C₅haloalkyl, —C₁alk-C≡C—C₅haloalkyl, —C₂alk-C≡C—C₅haloalkyl, —C₃alk-C≡C—C₅haloalkyl, —C₄alk-C≡C—C₅haloalkyl, —C₅alk-C≡C—C₅haloalkyl, —C₆alk-C≡C—C₅haloalkyl, —C₀alk-C≡C—C₆haloalkyl, —C₁alk-C≡C—C₆haloalkyl, —C₂alk-C≡C—C₆haloalkyl, —C₃alk-C≡C—C₆haloalkyl, —C₄alk-C≡C—C₆haloalkyl, —C₅alk-C≡C—C₆haloalkyl, —C₆alk-C≡C—C₆haloalkyl, —CH(OH)—C≡C—C₁-C₆haloalkyl, —CH(F)—C≡C—C₁-C₆haloalkyl, —CH(NH₂)—C≡C—C₁-C₆haloalkyl, —CH(Me)-C≡C—C₁-C₆haloalkyl, —C(Me)(OH)—C≡C—C₁-C₆haloalkyl, and the like. In some embodiments wherein —C₀-C₆alk-C≡C—C₁-C₆haloalkyl is —C₀-C₆alk-C≡C—CF₃, R¹ is —CH(OH)—C≡C—CF₃, —CH(F)—C≡C—CF₃, —CH(NH₂)—C≡C—CF₃, —CH(Me)-C≡C—CF₃, —C(Me)(OH)—C≡C—CF₃, and the like. Thus, in some embodiments, R¹ is —CH(OH)—C≡C—CF₃.

In some aspects, R¹ in Formula III or Formula IV is —C₀-C₆alk-C≡C—C₃-C₆cycloalkyl, for example, —C₀alk-C≡C—C₃cycloalkyl, —C₀alk-C≡C—C₄cycloalkyl, —C₀alk-C≡C—C₅cycloalkyl, —C₀alk-C≡C—C₆cycloalkyl, —C₁alk-C≡C—C₃cycloalkyl, —C₁alk-C≡C—C₄cycloalkyl, —C₁alk-C≡C—C₅-cycloalkyl, —C₁alk-C≡C—C₆cycloalkyl, —C₂alk-C≡C—C₃cycloalkyl, —C₂alk-C≡C—C₄cycloalkyl, —C₂alk-C≡C—C₅cycloalkyl, —C₂alk-C≡C—C₆cycloalkyl, —C₃alk-C≡C—C₃cycloalkyl, —C₃alk-C≡C—C₄cycloalkyl, —C₃alk-C≡C—C₅cycloalkyl, —C₃alk-C≡C—C₆cycloalkyl, —C₄alk-C≡C—C₃cycloalkyl, —C₄alk-C≡C—C₄cycloalkyl, —C₄alk-C≡C—C₅cycloalkyl, —C₄alk-C≡C—C₆cycloalkyl, —C₅alk-C≡C—C₃cycloalkyl, —C₅alk-C≡C—C₄cycloalkyl, —C₅alk-C≡C—C₅cycloalkyl, —C₅alk-C≡C—C₆cycloalkyl, —C₆alk-C≡C—C₃cycloalkyl, —C₆alk-C≡C—C₄cycloalkyl, —C₆alk-C≡C—C₅cycloalkyl, —C₆alk-C≡C—C₆cycloalkyl, —CH(OH)—C≡C—C₃-C₆cycloalkyl, —CH(F)—C≡C—C₃-C₆cycloalkyl, —CH(NH₂)—C≡C—C₃-C₆cycloalkyl, —CH(Me)-C≡C—C₃-C₆cycloalkyl, or —C(Me)(OH)—C≡C—C₃-C₆cycloalkyl. In some embodiments wherein —C₀-C₆alk-C≡C—C₃-C₆cycloalkyl is —C₀-C₆alk-C≡C-cyclopropyl, R¹ is —CH(OH)—C≡C-cyclopropyl, —CH(F)—C≡C-cyclopropyl, —CH(NH₂)—C≡C-cyclopropyl, —CH(Me)-C≡C-cyclopropyl, —C(Me)(OH)—C≡C-cyclopropyl, and the like. Thus, in some embodiments, R¹ is —CH(OH)—C≡C-cyclopropyl.

In some aspects, R¹ in Formula III or Formula IV is —C₁-C₆alk-aryl, for example, —C₁alk-aryl, —C₂alk-aryl, —C₃alk-aryl, —C₄alk-aryl, —C₅alk-aryl, —C₀alk-aryl, —CH₂aryl, —CH(OH)-aryl, —CH(F)-aryl, —CH(NH₂)-aryl, —CH(Me)-aryl, —C(Me)(OH)-aryl, and the like. In some embodiments wherein R¹ is —C₁-C₆alk-aryl, the -aryl is -4-chlorophenyl, -3,4-dichlorophenyl, -3,4-difluorophenyl, -3-fluoro-4-chlorophenyl, or -3-chloro-4-fluorophenyl. Thus in some embodiments, R¹ is —CH₂-difluorophenyl, —CH₂-3,4-difluorophenyl, —CH₂-4-chlorophenyl, —CH₂-3-chloro-4-fluorophenyl, —CH₂-4-chloro-3-fluorophenyl, —CH₂-dichlorophenyl, —CH₂-3,4-dichlorophenyl, —CH(OH)-4-chlorophenyl, —CH(OH)-3,4-dichlorophenyl, —CH(OH)-3,4-difluorophenyl, —CH(OH)-3-fluoro-4-chlorophenyl, —CH(OH)-3-chloro-4-fluorophenyl, —CH(F)-4-chlorophenyl, —CH(F)-3,4-dichlorophenyl, —CH(F)-3,4-difluorophenyl, —CH(F)-3-fluoro-4-chlorophenyl, —CH(F)-3-chloro-4-fluorophenyl, —CH(NH₂)-4-chlorophenyl, —CH(NH₂)-3,4-dichlorophenyl, —CH(NH₂)-3,4-difluorophenyl, —CH(NH₂)-3-fluoro-4-chlorophenyl, —CH(NH₂)-3-chloro-4-fluorophenyl, —CH(Me)-4-chlorophenyl, —CH(Me)-3,4-dichlorophenyl, —CH(Me)-3,4-difluorophenyl, —CH(Me)-3-fluoro-4-chlorophenyl, —CH(Me)-3-chloro-4-fluorophenyl, —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, or —C(Me)(OH)-3-chloro-4-fluorophenyl.

In some aspects, R¹ in Formula III or Formula IV is —C₁-C₆alk-S—C₁-C₆alkyl, for example —C₁alk-S—C₁alkyl, —C₂alk-S—C₁alkyl, —C₃alk-S—C₁alkyl, —C₄alk-S—C₁alkyl, —C₅alk-S—C₁alkyl, —C₆alk-S—C₁alkyl, —C₁alk-S—C₂alkyl, —C₂alk-S—C₂alkyl, —C₃alk-S—C₂alkyl, —C₄alk-S—C₂alkyl, —C₅alk-S—C₂alkyl, —C₆alk-S—C₂alkyl, —C₁alk-S—C₃alkyl, —C₂alk-S—C₃alkyl, —C₃alk-S—C₃alkyl, —C₄alk-S—C₃alkyl, —C₅alk-S—C₃alkyl, —C₆alk-S—C₃alkyl, —C₁alk-S—C₄alkyl, —C₂alk-S—C₄alkyl, —C₃alk-S—C₄alkyl, —C₄alk-S—C₄alkyl, —C₅alk-S—C₄alkyl, —C₆alk-S—C₄alkyl, —C₁alk-S—C₅alkyl, —C₂alk-S—C₅alkyl, —C₃alk-S—C₅alkyl, —C₄alk-S—C₅alkyl, —C₅alk-S—C₅alkyl, —C₆alk-S—C₅alkyl, —C₁alk-S—C₆alkyl, —C₂alk-S—C₆alkyl, —C₃alk-S—C₆alkyl, —C₄alk-S—C₆alkyl, —C₅alk-S—C₆alkyl, —C₆alk-S—C₆alkyl, —CH₂S—C₂alkyl, —CH₂S—C₃alkyl, —CH₂S—C₄alkyl, —CH₂S—C₅alkyl, —CH₂S—C₆alkyl, and the like. Thus, in some aspects R¹ is —CH₂S—C₁alkyl. In some aspects, R¹ is —CH₂—S—CH₃.

In some aspects, R¹ in Formula III or Formula IV is —C₁-C₆alk-S—C₁-C₆haloalkyl, for example —C₁alk-S—C₁haloalkyl, —C₂alk-S—C₁haloalkyl, —C₃alk-S—C₁haloalkyl, —C₄alk-S—C₁haloalkyl, —C₅alk-S—C₁haloalkyl, —C₆alk-S—C₁haloalkyl, —C₁alk-S—C₂haloalkyl, —C₂alk-S—C₂haloalkyl, —C₃alk-S—C₂haloalkyl, —C₄alk-S—C₂haloalkyl, —C₅alk-S—C₂haloalkyl, —C₆alk-S—C₂haloalkyl, —C₁alk-S—C₃haloalkyl, —C₂alk-S—C₃haloalkyl, —C₃alk-S—C₃haloalkyl, —C₄alk-S—C₃haloalkyl, —C₅alk-S—C₃haloalkyl, —C₆alk-S—C₃haloalkyl, —C₁alk-S—C₄haloalkyl, —C₂alk-S—C₄haloalkyl, —C₃alk-S—C₄haloalkyl, —C₄alk-S—C₄haloalkyl, —C₅alk-S—C₄haloalkyl, —C₆alk-S—C₄haloalkyl, —C₁alk-S—C₅haloalkyl, —C₂alk-S—C₅haloalkyl, —C₃alk-S—C₅haloalkyl, —C₄alk-S—C₅haloalkyl, —C₅alk-S—C₅haloalkyl, —C₆alk-S—C₅haloalkyl, —C₁alk-S—C₆haloalkyl, —C₂alk-S—C₆haloalkyl, —C₃alk-S—C₆haloalkyl, —C₄alk-S—C₆haloalkyl, —C₅alk-S—C₆haloalkyl, —C₆alk-S—C₆haloalkyl, —CH₂S—C₁haloalkyl, —CH₂S—C₂haloalkyl, —CH₂S—C₃haloalkyl, —CH₂S—C₄haloalkyl, —CH₂S—C₅haloalkyl, and —CH₂S—C₆haloalkyl.

In some aspects, R¹ in Formula III or Formula IV is —C₁-C₆alk-S—C₃-C₆cycloalkyl, for example —C₁alk-S—C₃cycloalkyl, —C₂alk-S—C₃cycloalkyl, —C₃alk-S—C₃cycloalkyl, —C₄alk-S—C₃cycloalkyl, —C₅alk-S—C₃cycloalkyl, —C₆alk-S—C₃cycloalkyl, —C₁alk-S—C₄cycloalkyl, —C₂alk-S—C₄cycloalkyl, —C₃alk-S—C₄cycloalkyl, —C₄alk-S—C₄cycloalkyl, —C₅alk-S—C₄cycloalkyl, —C₆alk-S—C₄cycloalkyl, —C₁alk-S—C₅cycloalkyl, —C₂alk-S—C₅cycloalkyl, —C₃alk-S—C₅cycloalkyl, —C₄alk-S—C₅cycloalkyl, —C₅alk-S—C₅cycloalkyl, —C₆alk-S—C₅cycloalkyl, —C₁alk-S—C₆cycloalkyl, —C₂alk-S—C₆cycloalkyl, —C₃alk-S—C₆cycloalkyl, —C₄alk-S—C₆cycloalkyl, —C₅alk-S—C₆cycloalkyl, —C₆alk-S—C₆cycloalkyl, —CH₂S—C₃cycloalkyl, —CH₂S—C₄cycloalkyl, —CH₂S—C₅cycloalkyl, —CH₂S—C₆cycloalkyl, and the like.

In some aspects, R¹ in Formula III or Formula IV is —C₁-C₆alk-S—C₃-C₆halocycloalkyl, for example —C₁alk-S—C₃halocycloalkyl, —C₂alk-S—C₃halocycloalkyl, —C₃alk-S—C₃halocycloalkyl, —C₄alk-S—C₃halocycloalkyl, —C₅alk-S—C₃halocycloalkyl, —C₆alk-S—C₃halocycloalkyl, —C₁alk-S—C₄halocycloalkyl, —C₂alk-S—C₄halocycloalkyl, —C₃alk-S—C₄halocycloalkyl, —C₄alk-S—C₄halocycloalkyl, —C₅alk-S—C₄halocycloalkyl, —C₆alk-S—C₄halocycloalkyl, —C₁alk-S—C₅halocycloalkyl, —C₂alk-S—C₅halocycloalkyl, —C₃alk-S—C₅halocycloalkyl, —C₄alk-S—C₅halocycloalkyl, —C₅alk-S—C₅halocycloalkyl, —C₆alk-S—C₅halocycloalkyl, —C₁alk-S—C₆halocycloalkyl, —C₂alk-S—C₆halocycloalkyl, —C₃alk-S—C₆halocycloalkyl, —C₄alk-S—C₆halocycloalkyl, —C₅alk-S—C₆halocycloalkyl, —C₆alk-S—C₆halocycloalkyl, —CH₂S—C₃halocycloalkyl, —CH₂S—C₄halocycloalkyl, —CH₂S—C₅halocycloalkyl, —CH₂S—C₆halocycloalkyl, and the like.

In some aspects, R¹ in Formula III or Formula IV is —C₁-C₆alk-O—C₁-C₆alkyl, for example, —C₁alk-O—C₁alkyl, —C₂alk-O—C₁alkyl, —C₃alk-O—C₁alkyl, —C₄alk-O—C₁alkyl, —C₅alk-O—C₁alkyl, —C₆alk-O—C₁alkyl, —C₁alk-O—C₂alkyl, —C₂alk-O—C₂alkyl, —C₃alk-O—C₂alkyl, —C₄alk-O—C₂alkyl, —C₅alk-O—C₂alkyl, —C₆alk-O—C₂alkyl, —C₁alk-O—C₃alkyl, —C₂alk-O—C₃alkyl, —C₃alk-O—C₃alkyl, —C₄alk-O—C₃alkyl, —C₅alk-O—C₃alkyl, —C₆alk-O—C₃alkyl, —C₁alk-O—C₄alkyl, —C₂alk-O—C₄alkyl, —C₃alk-O—C₄alkyl, —C₄alk-O—C₄alkyl, —C₅alk-O—C₄alkyl, —C₆alk-O—C₄alkyl, —C₁alk-O—C₅alkyl, —C₂alk-O—C₅alkyl, —C₃alk-O—C₅alkyl, —C₄alk-O—C₅alkyl, —C₅alk-O—C₅alkyl, —C₆alk-O—C₅alkyl, —C₁alk-O—C₆alkyl, —C₂alk-O—C₆alkyl, —C₃alk-O—C₆alkyl, —C₄alk-O—C₆alkyl, —C₅alk-O—C₆alkyl, —C₀alk-O—C₆alkyl, —CH₂OC₁alkyl, —CH₂OC₂alkyl, —CH₂OC₃alkyl, —CH₂OC₄alkyl, —CH₂OC₅alkyl, —CH₂OC₆alkyl, and the like.

In some aspects, R¹ in Formula III or Formula IV is —C₁-C₆alk-O—C₃-C₆cycloalkyl, for example, —C₁alk-O—C₃cycloalkyl, —C₂alk-O—C₃cycloalkyl, —C₃alk-O—C₃cycloalkyl, —C₄alk-O—C₃cycloalkyl, —C₅alk-O—C₃cycloalkyl, —C₆alk-O—C₃cycloalkyl, —C₁alk-O—C₄cycloalkyl, —C₂alk-O—C₄cycloalkyl, —C₃alk-O—C₄cycloalkyl, —C₄alk-O—C₄cycloalkyl, —C₅alk-O—C₄cycloalkyl, —C₆alk-O—C₄cycloalkyl, —C₁alk-O—C₅cycloalkyl, —C₂alk-O—C₅cycloalkyl, —C₃alk-O—C₅cycloalkyl, —C₄alk-O—C₅cycloalkyl, —C₅alk-O—C₅cycloalkyl, —C₆alk-O—C₅cycloalkyl, —C₁alk-O—C₆cycloalkyl, —C₂alk-O—C₆cycloalkyl, —C₃alk-O—C₆cycloalkyl, —C₄alk-O—C₆cycloalkyl, —C₅alk-O—C₆cycloalkyl, —C₆alk-O—C₆cycloalkyl, —CH₂O—C₆cycloalkyl, —CH₂O—C₅cycloalkyl, —CH₂O—C₄cycloalkyl, —CH₂O—C₃cycloalkyl, —CH₂O—C₆cycloalkyl, and the like.

In some aspects, R¹ in Formula III or Formula IV is —C₁-C₆alk-SCH₂-aryl, for example —C₁alk-SCH₂-aryl, —C₂alk-SCH₂-aryl, —C₃alk-SCH₂-aryl, —C₄alk-SCH₂-aryl, —C₅alk-SCH₂-aryl, —C₆alk-SCH₂-aryl, —CH₂SCH₂-phenyl, —CH₂SCH₂-naphthyl, —CH₂SCH₂-fluorophenyl, —CH₂SCH₂-difluorophenyl, —CH₂SCH₂-fluoronaphthyl, —CH₂SCH₂-chlorophenyl, —CH₂SCH₂-bromophenyl, —CH₂SCH₂-iodophenyl, —CH₂SCH₂-methylphenyl, —CH₂SCH₂-4-chlorophenyl, —CH₂SCH₂-3,4-dichlorophenyl, —CH₂SCH₂-3,4-difluorophenyl, —CH₂SCH₂-3-fluoro-4-chlorophenyl, —CH₂SCH₂-3-chloro-4-fluorophenyl, and the like. Thus, in some aspects R¹ is —CH₂SCH₂-phenyl.

In some aspects, R¹ in Formula III or Formula IV is —C₁-C₆alkC(O)NH-aryl, for example, —C₁alk-C(O)NH-aryl, —C₂alk-C(O)NH-aryl, —C₃alk-C(O)NH-aryl, —C₄alk-C(O)NH-aryl, —C₅alk-C(O)NH-aryl, —C₆alk-C(O)NH-aryl, —CH₂C(O)NH-phenyl, —CH₂C(O)NH-naphthyl, —CH₂C(O)NH-fluorophenyl, —CH₂C(O)NH-difluorophenyl, —CH₂C(O)NH-fluoronaphthyl, —CH₂C(O)NH-chlorophenyl, —CH₂C(O)NH-bromophenyl, —CH₂C(O)NH-iodophenyl, —CH₂C(O)NH— methylphenyl, —CH₂C(O)NH-4-chlorophenyl, —CH₂C(O)NH-3,4-dichlorophenyl, —CH₂C(O)NH-3,4-difluorophenyl, —CH₂C(O)NH-3-fluoro-4-chlorophenyl, —CH₂C(O)NH-3-chloro-4-fluorophenyl and the like. Thus, in some aspects R¹ is —CH₂C(O)NH-phenyl.

In some aspects, R¹ in Formula III or Formula IV is —C₀-C₆alk-S-aryl, for example, —C₀alk-S-aryl, —C₁alk-S-aryl, —C₂alk-S-aryl, —C₃alk-S-aryl, —C₄alk-S-aryl, —C₅alk-S-aryl, —C₀alk-S-aryl, —S-phenyl, —S-naphthyl, —S-fluorophenyl, —S-difluorophenyl, —S-fluoronaphthyl, —S-chlorophenyl, —S— bromophenyl, —S-iodophenyl, —S-methylphenyl, and the like. In some aspects R¹ is —S— difluorophenyl. In some aspects R¹ is —S-3,4-difluorophenyl. In other aspects, R¹ is —S-chlorophenyl. In other aspects, R¹ is —S-4-chlorophenyl. In other aspects, R¹ is —S-chlorofluorophenyl. In other aspects, R¹ is —S-3-chloro-4-fluorophenyl. In other aspects, R¹ is —S-4-chloro-3-fluorophenyl. In other aspects, R¹ is —S-dichlorophenyl. In other aspects, R¹ is —S-3,4-dichlorophenyl.

In some aspects, R¹ in Formula III or Formula IV is —C₀-C₆alk-S(O)aryl, for example, —C₀alk-S(O)aryl, —C₁alk-S(O)aryl, —C₂alk-S(O)aryl, —C₃alk-S(O)aryl, —C₄alk-S(O)aryl, —C₅alk-S(O)aryl, —C₀alk-S(O)aryl, —S(O)-phenyl, —S(O)-naphthyl, —S(O)-fluorophenyl, —S(O)— difluorophenyl, —S(O)-fluoronaphthyl, —S(O)-chlorophenyl, —S(O)-bromophenyl, —S(O)-iodophenyl, —S(O)-methylphenyl, and the like. In some aspects R¹ is —S(O)-difluorophenyl. In some aspects R¹ is —S(O)-3,4-difluorophenyl. In other aspects, R¹ is —S(O)-chlorophenyl. In other aspects, R¹ is —S(O)-4-chlorophenyl. In other aspects, R¹ is —S(O)-chlorofluorophenyl. In other aspects, R¹ is —S(O)-3-chloro-4-fluorophenyl. In other aspects, R¹ is —S(O)-4-chloro-3-fluorophenyl. In other aspects, R¹ is —S(O)-dichlorophenyl. In other aspects, R¹ is —S(O)-3,4-dichlorophenyl.

In some aspects, R¹ in Formula III or Formula IV is —C₀-C₆alk-S(O)₂aryl, for example, —C₀alk-S(O)₂aryl, —C₁alk-S(O)₂aryl, —C₂alk-S(O)₂aryl-C₃alk-S(O)₂aryl, —C₄alk-S(O)₂aryl, —C₅alk-S(O)₂aryl, —C₆alk-S(O)₂aryl, —S(O)₂-phenyl, —S(O)₂-naphthyl, —S(O)₂-fluorophenyl, —S(O)₂-difluorophenyl, —S(O)₂-fluoronaphthyl, —S(O)₂-chlorophenyl, —S(O)₂-bromophenyl, —S(O)₂-iodophenyl, —S(O)₂-methylphenyl, and the like. In some aspects R¹ is —S(O)₂-difluorophenyl. In some aspects R¹ is —S(O)₂-3,4-difluorophenyl. In other aspects, R¹ is —S(O)₂-chlorophenyl. In other aspects, R¹ is —S(O)₂-4-chlorophenyl. In other aspects, R¹ is —S(O)₂-chlorofluorophenyl. In other aspects, R¹ is —S(O)₂-3-chloro-4-fluorophenyl. In other aspects, R¹ is —S(O)₂-4-chloro-3-fluorophenyl. In other aspects, R¹ is —S(O)₂-dichlorophenyl. In other aspects, R¹ is —S(O)₂-3,4-dichlorophenyl.

In some aspects, R¹ in Formula III or Formula IV is —C₀-C₆alk-Oaryl, for example —C₀alk-Oaryl, —C₁alk-Oaryl, —C₂alk-Oaryl —C₃alk-Oaryl, —C₄alk-Oaryl, —C₅alk-Oaryl, —C₀alk-Oaryl, —O-phenyl, —O-naphthyl, —O-fluorophenyl, —O-difluorophenyl, —O-fluoronaphthyl, —O-chlorophenyl, —O-bromophenyl, —O-iodophenyl, —O-methylphenyl, and the like. In some aspects R¹ is —O-difluorophenyl. In some aspects R¹ is —O-3,4-difluorophenyl. In other aspects, R¹ is —O-chlorophenyl. In other aspects, R¹ is —O-4-chlorophenyl. In other aspects, R¹ is —O-chlorofluorophenyl. In other aspects, R¹ is —O-3-chloro-4-fluorophenyl. In other aspects, R¹ is —O-4-chloro-3-fluorophenyl. In other aspects, R¹ is —O-dichlorophenyl. In other aspects, R¹ is —O-3,4-dichlorophenyl.

In some aspects, R¹ in Formula III or Formula IV is —C₀-C₆alk-heteroaryl, for example, —C₀alk-heteroaryl, —C₁alk-heteroaryl, —C₂alk-heteroaryl, —C₃alk-heteroaryl, —C₄alk-heteroaryl, —C₅alk-heteroaryl, and —C₀alk-heteroaryl. In some aspects, R¹ is 2-(2-amino-3-bromoquinolin-7-yl)ethyl, 2-(2-amino-3-chloroquinolin-7-yl)ethyl, 2-(2-((cyclopropylmethyl)amino)quinolin-7-yl)ethyl, 2-(2-(methylamino)quinolin-7-yl)ethyl, or 2-(2-aminoquinolin-7-yl)ethyl.

In some aspects, R¹ in Formula III or Formula IV is —C₁-C₆alk-O-heteroaryl, for example, —C₁alk-O-heteroaryl, —C₂alk-O-heteroaryl, —C₃alk-O-heteroaryl, —C₄alk-O-heteroaryl, —C₅alk-O-heteroaryl, and —C₆alk-O-heteroaryl. In some aspects, R¹ is 2-amino-3-bromoquinolin-7-yl)oxy)methyl.

In some aspects, R¹ in Formula III or Formula IV is —C₁-C₆alk-S-heteroaryl, for example, —C₁alk-S-heteroaryl, —C₂alk-S-heteroaryl, —C₃alk-S-heteroaryl, —C₄alk-S-heteroaryl, —C₅alk-S-heteroaryl, and —C₀alk-S-heteroaryl. In some aspects, R¹ is 2-amino-3-bromoquinolin-7-yl)thio)methyl.

In some aspects, R¹ in Formula III or Formula IV is —C₁-C₆alk-NH-heteroaryl, for example, —C₁alk-NH-heteroaryl, —C₂alk-NH-heteroaryl, —C₃alk-NH-heteroaryl, —C₄alk-NH-heteroaryl, —C₅alk-NH-heteroaryl, and —C₀alk-NH-heteroaryl. In some aspects, R¹ is 2-amino-3-bromoquinolin-7-yl)amino)methyl.

In some aspects of Formula III or Formula IV, R² is H, —C₁-C₆alkyl, —C₁-C₆haloalkyl, or —C₀-C₆alk-C₃-C₆cycloalkyl. Thus, in some embodiments, R² is H.

It will be apparent that when R² is H, the compounds of Formula III or Formula IV may exist in equivalent, tautomeric forms. Thus, when R² is H, compounds of Formula III may be represented by either of the following equivalent structures:

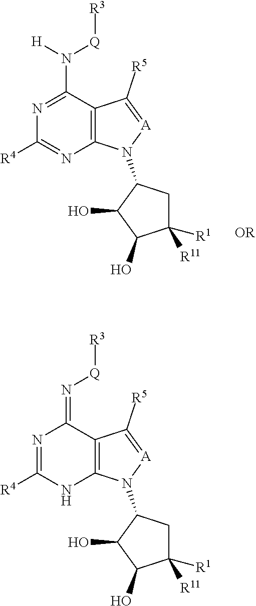

Similarly, when R² is H, compounds of Formula IV may be represented by the following equivalent structures:

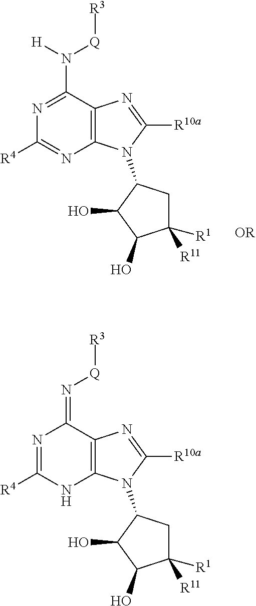

It will be apparent that in some tautomeric forms of the compounds of Formula III or Formula IV, geometric isomerism in the exocyclic carbon-nitrogen double bond can result in E- and Z-isomers, as shown below:

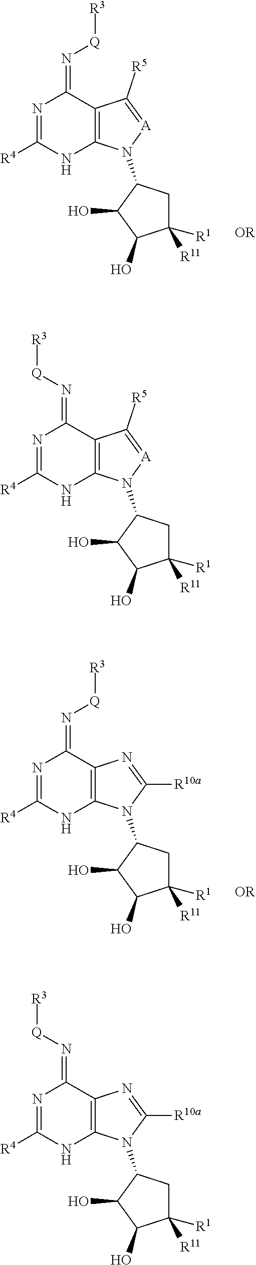

The compounds of Formula III or Formula IV described and claimed herein are meant to encompass both E- and Z-geometric isomers. The depiction of a particular geometric isomer is not intended to be limiting.

In some embodiments, R² in Formula III or Formula IV is —C₁-C₆alkyl, for example, methyl, ethyl, propyl, isopropyl, butyl, isobutyl, s-butyl, t-butyl, pentyl, and the like. Thus, in some embodiments, R² is methyl (i.e., —CH₃, or Me).

In some aspects, R² in Formula III or Formula IV is —C₁-C₆haloalkyl, for example, —CF₃ or —CH₂ and the like.

In some aspects, R² in Formula III or Formula IV is —C₀-C₆alk-C₃-C₆cycloalkyl, for example, —C₀alk-C₃cycloalkyl, —C₁alk-C₃cycloalkyl, —C₂alk-C₃cycloalkyl, —C₃alk-C₃cycloalkyl, —C₄alk-C₃cycloalkyl, —C₅alk-C₃cycloalkyl, —C₆alk-C₃cycloalkyl, —C₀alk-C₄cycloalkyl, —C₁alk-C₄cycloalkyl, —C₂alk-C₄cycloalkyl, —C₃alk-C₄cycloalkyl, —C₄alk-C₄cycloalkyl, —C₅alk-C₄cycloalkyl, —C₆alk-C₄cycloalkyl, —C₀alk-C₅cycloalkyl, —C₁alk-C₅cycloalkyl, —C₂alk-C₅cycloalkyl, —C₃alk-C₅cycloalkyl, —C₄alk-C₅cycloalkyl, —C₅alk-C₅cycloalkyl, —C₆alk-C₅cycloalkyl, —C₀alk-C₆cycloalkyl, —C₁alk-C₆cycloalkyl, —C₂alk-C₆cycloalkyl, —C₃alk-C₆cycloalkyl, —C₄alk-C₆cycloalkyl, —C₅alk-C₆cycloalkyl, or —C₆alk-C₆cycloalkyl. In some aspects wherein R² is —C₀-C₆alk-C₃-C₆cycloalkyl, the cycloalkyl is unsubstituted. In other aspects wherein R² is —C₀-C₆alk-C₃-C₆cycloalkyl, the cycloalkyl is substituted with one, two, or three R substituents independently selected from C₁-C₆alkyl, (e.g., methyl, ethyl, propyl, isopropyl, butyl), —OC₁-C₆alkyl (e.g., —Omethyl, —Oethyl, —Opropyl, —Oisopropyl, —Obutyl), and halo (e.g., F or Cl).

In some aspects of Formula III or Formula IV, R³ is H, —C₁-C₆alkyl, —C₁-C₆haloalkyl, —C₀-C₆alk-C₃-C₆cycloalkyl, —C(O)R⁷, —C(O)OR⁷, or —C(O)NR^{8a}R^{8b}. Thus, in some embodiments, R³ is H.

In some aspects, R³ in Formula III or Formula IV is —C₁-C₆alkyl, for example, methyl, ethyl, propyl, isopropyl, butyl, isobutyl, s-butyl, t-butyl, pentyl, and the like. Thus, in some embodiments, R³ is methyl. In other embodiments, R³ is ethyl.

In some aspects, R³ in Formula III or Formula IV is —C₁-C₆haloalkyl, for example, —CF₃, —CH₂CF₃, —CH₂CHF₂ or —CH₂ and the like.

In some aspects, R³ in Formula III or Formula IV is —C₀-C₆alk-C₃-C₆cycloalkyl, for example, —C₀alk-C₃cycloalkyl, —C₁alk-C₃cycloalkyl, —C₂alk-C₃cycloalkyl, —C₃alk-C₃cycloalkyl, —C₄alk-C₃cycloalkyl, —C₅alk-C₃cycloalkyl, —C₆alk-C₃cycloalkyl, —C₀alk-C₄cycloalkyl, —C₁alk-C₄cycloalkyl, —C₂alk-C₄cycloalkyl, —C₃alk-C₄cycloalkyl, —C₄alk-C₄cycloalkyl, —C₅alk-C₄cycloalkyl, —C₆alk-C₄cycloalkyl, —C₀alk-C₅cycloalkyl, —C₁alk-C₅cycloalkyl, —C₂alk-C₅cycloalkyl, —C₃alk-C₅cycloalkyl, —C₄alk-C₅cycloalkyl, —C₅alk-C₅cycloalkyl, —C₆alk-C₅cycloalkyl, —C₀alk-C₆cycloalkyl, —C₁alk-C₆cycloalkyl, —C₂alk-C₆cycloalkyl, —C₃alk-C₆cycloalkyl, —C₄alk-C₆cycloalkyl, —C₅alk-C₆cycloalkyl, —C₆alk-C₆cycloalkyl. In some aspects wherein R³ is —C₀-C₆alk-C₃-C₆cycloalkyl, the cycloalkyl is unsubstituted. In other aspects wherein R³ is —C₀-C₆alk-C₃-C₆cycloalkyl, the cycloalkyl is substituted with one, two, or three R substituents independently selected from C₁-C₆alkyl, (e.g., methyl, ethyl, propyl, isopropyl, butyl), —OC₁-C₆alkyl (e.g., —Omethyl, —Oethyl, —Opropyl, —Oisopropyl, —Obutyl), and halo (e.g., F or Cl).

In some aspects, R³ in Formula III or Formula IV is —C(O)R⁷ or —C(O)OR⁷. In these embodiments, R⁷ is H, C₁-C₆alkyl, or C₀-C₆alk-C₃-C₆cycloalkyl.

In some aspects, R⁷ in Formula III or Formula IV is H, C₁-C₆alkyl, or C₀-C₆alk-C₃-C₆cycloalkyl. Thus, in some aspects, R⁷ is H.

In other aspects, R⁷ in Formula III or Formula IV is C₁-C₆alkyl, for example, methyl, ethyl, propyl, isopropyl, butyl, isobutyl, s-butyl, t-butyl, pentyl, and the like. Thus, in some embodiments, R⁷ is methyl.

In other aspects, R⁷ in Formula III or Formula IV is —C₀-C₆alk-C₃-C₆cycloalkyl, for example, —C₀alk-C₃cycloalkyl, —C₁alk-C₃cycloalkyl, —C₂alk-C₃cycloalkyl, —C₃alk-C₃cycloalkyl, —C₄alk-C₃cycloalkyl, —C₅alk-C₃cycloalkyl, —C₆alk-C₃cycloalkyl, —C₀alk-C₄cycloalkyl, —C₁alk-C₄cycloalkyl, —C₂alk-C₄cycloalkyl, —C₃alk-C₄cycloalkyl, —C₄alk-C₄cycloalkyl, —C₅alk-C₄cycloalkyl, —C₆alk-C₄cycloalkyl, —C₀alk-C₅cycloalkyl, —C₁alk-C₅cycloalkyl, —C₂alk-C₅cycloalkyl, —C₃alk-C₅cycloalkyl, —C₄alk-C₅cycloalkyl, —C₅alk-C₅cycloalkyl, —C₆alk-C₅cycloalkyl, —C₀alk-C₆cycloalkyl, —C₁alk-C₆cycloalkyl, —C₂alk-C₆cycloalkyl, —C₃alk-C₆cycloalkyl, —C₄alk-C₆cycloalkyl, —C₅alk-C₆cycloalkyl, and —C₆alk-C₆cycloalkyl.

In some aspects, R³ in Formula III or Formula IV is —C(O)R⁷. In some embodiments wherein R⁷ is —C₁-C₆alkyl, R³ is —C(O)C₁-C₆alkyl. Thus, in some embodiments wherein R⁷ is methyl, R³ is acetyl (i.e., —C(O)CH₃).

In some aspects, R³ in Formula III or Formula IV is —C(O)OR⁷. In some embodiments wherein R⁷ is —C₁-C₆alkyl, R³ is —C(O)OC₁-C₆alkyl. Thus, in some embodiments wherein R⁷ is methyl, R³ is —C(O)OCH₃.

In some aspects, R³ in Formula III or Formula IV is or —C(O)NR^{8a}R^{8b}.

In some aspects, R^{8a} and R^{8b} in Formula III or Formula IV are each independently H, C₁-C₆alkyl (e.g., methyl, ethyl, propyl, isopropyl, butyl, isobutyl, s-butyl, t-butyl, pentyl, and the like), or —C₀-C₆alk-O—C₁-C₆alkyl (e.g., —C₀alk-O—C₁alkyl, —C₁alk-O—C₁alkyl, —C₂alk-O—C₁alkyl, —C₃alk-O—C₁alkyl, —C₄alk-O—C₁alkyl, —C₅alk-O—C₁alkyl, —C₀alk-O—C₁alkyl, —C₀alk-O—C₂alkyl, —C₁alk-O—C₂alkyl, —C₂alk-O—C₂alkyl, —C₃alk-O—C₂alkyl, —C₄alk-O—C₂alkyl, —C₅alk-O—C₂alkyl, —C₆alk-O—C₂alkyl, —C₀alk-O—C₃alkyl, —C₁alk-O—C₃alkyl, —C₂alk-O—C₃alkyl, —C₃alk-O—C₃alkyl, —C₄alk-O—C₃alkyl, —C₅alk-O—C₃alkyl, —C₆alk-O—C₃alkyl, —C₀alk-O—C₄alkyl, —C₁alk-O—C₄alkyl, —C₂alk-O—C₄alkyl, —C₃alk-O—C₄alkyl, —C₄alk-O—C₄alkyl, —C₅alk-O—C₄alkyl, —C₆alk-O—C₄alkyl, —C₀alk-O—C₅alkyl, —C₁alk-O—C₅alkyl, —C₂alk-O—C₅alkyl, —C₃alk-O—C₅alkyl, —C₄alk-O—C₅alkyl, —C₅alk-O—C₅alkyl, —C₆alk-O—C₅alkyl, —C₀alk-O—C₆alkyl, —C₁alk-O—C₆alkyl, —C₂alk-O—C₆alkyl, —C₃alk-O—C₆alkyl, —C₄alk-O—C₆alkyl, —C₅alk-O—C₆alkyl, —C₀alk-O—C₆alkyl). In some embodiments, R^{8a} is C₁-C₆alkyl or —C₀-C₆alk-OC₁-C₆alkyl and R^{8b} is H, C₁-C₆alkyl, and —C₀-C₆alk-OC₁-C₆alkyl.

In some embodiments, R^{8a} in Formula III or Formula IV is H or C₁-C₆alkyl. In some embodiments, R^{8b} is H or C₁-C₆alkyl. In some embodiments, R^{8a} and R^{8b} are each H. In other embodiments, R^{8a} and R^{8b} are each independently C₁-C₆alkyl. In some aspects, R^{8a} is C₁-C₆alkyl and R^{8b} is H.

In other aspects, R^{8a} and R^{8b} in Formula III or Formula IV are each independently —C₀-C₆alk-OC₁-C₆alkyl.

In other aspects, R^{8a} in Formula III or Formula IV is —C₀-C₆alk-OC₁-C₆alkyl and R^{8b} is H.

In yet other aspects, R^{8a} and R^{8b} in Formula III or Formula IV, together with the atom to which they are attached, form a C₂-C₆heterocycloalkyl ring, for example, aziridinyl, azetidinyl, pyrrolidinyl, piperidinyl, and the like.

In some aspects of Formula III or Formula IV, R⁴ is H, halo, —C₁-C₆alkyl, or NH₂. Thus in some embodiments, R⁴ is H. In other embodiments, R⁴ is halo, for example F, Cl, Br, or I, with —Cl being preferred. In other embodiments, R⁴ is —C₁-C₆alkyl, for example, methyl, ethyl, propyl, isopropyl, butyl, isobutyl, s-butyl, t-butyl, pentyl, and the like. In yet other embodiments, R⁴ is NH₂.

In some aspects of Formula III, R⁵ is H, halo, CN, —C₁-C₆alkyl, —C₂-C₄alkenyl, —C₂-C₄haloalkenyl, C₂-C₄cyanoalkenyl, —C₀-C₆alk-C≡CH, —C₀-C₆alk-C≡C—C₁-C₆alkyl, —C₁-C₄haloalkyl, —C₂-C₆heterocycloalkyl, oxo-substituted-C₂-C₆heterocycloalkyl, —C₃-C₆cycloalkyl, —CHO, —C(O)R⁹, —CR⁸R^{8′}CN, —CH₂NR⁸R^{8′}, —C₀-C₆alk-OH, —NR⁸R^{8′}, —NH—CN, —N(R⁹)CN, —O—C₁-C₄alkyl, —NR⁹CONR⁸R^{8′}, —OCONR⁸R^{8′}, or —NR⁹C(O)OR^{9a}.

In some emodiments of Formula III, R⁵ is H.

In some embodiments of Formula III, R⁵ is halo, for example, F, Cl, Br, or I. Thus, in some embodiments, R⁵ is F.

In some emodiments of Formula III, R⁵ is CN.

In other embodiments of Formula III, R⁵ is —C₁-C₆alkyl, for example, methyl, ethyl, propyl, isopropyl, butyl, isobutyl, s-butyl, t-butyl, pentyl, and the like. Thus, in some aspects, R⁵ is methyl.

In some aspects, R⁵ in Formula III is —C₂-C₄alkenyl, for example, vinyl, allyl, and the like. Thus, in some embodiments, R⁵ is vinyl (—CH═CH₂).

In some aspects, R⁵ in Formula III is —C₂-C₄haloalkenyl, for example, —C(F)═CH₂, C(CF₃)═CH₂, and the like. Thus, in some embodiments, R⁵ is —C(F)═CH₂.

In other aspects, R⁵ in Formula III is —C₂-C₄cyanoalkenyl, for example, —C(CN)═CH₂, —CH═CHCN, and the like. Thus, in some embodiments, R⁵ is —C(CN)═CH₂.

In other embodiments, R⁵ in Formula III is —C₀-C₆alk-C≡CH, for example, —C₀alk-C≡CH, —C₁alk-C≡CH, —C₂alk-C≡CH, —C₃alk-C≡CH, —C₄alk-C≡CH, —C₅alk-C≡CH, —C₀alk-C≡CH, ethynyl, propargyl, and the like. Thus, in some embodiments, R⁵ is ethynyl (—C≡CH).

In some aspects, R⁵ in Formula III is —C₀-C₆alk-C≡C—C₁-C₆alkyl, for example, —C₀alk-C≡C—C₁alkyl, —C₁alk-C≡C—C₁alkyl, —C₂alk-C≡C—C₁alkyl, —C₃alk-C≡C—C₁alkyl, —C₄alk-C≡C—C₁alkyl, —C₅alk-C≡C—C₁alkyl, —C₀alk-C≡C—C₁alkyl, —C₀alk-C≡C—C₂alkyl, —C₁alk-C≡C—C₂alkyl, —C₂alk-C≡C—C₂alkyl, —C₃alk-C≡C—C₂alkyl, —C₄alk-C≡C—C₂alkyl, —C₅alk-C≡C—C₂alkyl, —C₀alk-C≡C—C₂alkyl, —C₀alk-C≡C—C₃alkyl, —C₁alk-C≡C—C₃alkyl, —C₂alk-C≡C—C₃alkyl, —C₃alk-C≡C—C₃alkyl, —C₄alk-C≡C—C₃alkyl, —C₅alk-C≡C—C₃alkyl, —C₆alk-C≡C—C₃alkyl, —C₀alk-C≡C—C₄alkyl, —C₁alk-C≡C—C₄alkyl, —C₂alk-C≡C—C₄alkyl, —C₃alk-C≡C—C₄alkyl, —C₄alk-C≡C—C₄alkyl, —C₅alk-C≡C—C₄alkyl, —C₆alk-C≡C—C₄alkyl, —C₀alk-C≡C—C₅alkyl, —C₁alk-C≡C—C₅alkyl, —C₂alk-C≡C—C₅alkyl, —C₃alk-C≡C—C₅alkyl, —C₄alk-C≡C—C₅alkyl, —C₅alk-C≡C—C₅alkyl, —C₆alk-C≡C—C₅alkyl, —C₀alk-C≡C—C₆alkyl, —C₁alk-C≡C—C₆alkyl, —C₂alk-C≡C—C₆alkyl, —C₃alk-C≡C—C₆alkyl, —C₄alk-C≡C—C₆alkyl, —C₅alk-C≡C—C₆alkyl, or —C₀alk-C≡C—C₆alkyl.

In some embodiments, R⁵ in Formula III is —C₁-C₄haloalkyl, for example, —CF₃ or —CHF₂.

In some embodiments, R⁵ in Formula III is —C₂-C₆heterocycloalkyl, for example C₂heterocycloalkyl, C₃heterocycloalkyl, C₄ heterocycloalkyl, C₅ heterocycloalkyl, and C₆ heterocycloalkyl, including azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, tetrahydrofuranyl, tetrahydropyranyl, piperazinyl, and the like. Thus, in some embodiments, R⁵ is 2-oxiranyl. In other embodiments, R⁵ is 1-azetidinyl.

In some embodiments, R⁵ in Formula III is oxo-substituted-C₂-C₆heterocycloalkyl, for example, oxo-substituted-C₂heterocycloalkyl, oxo-substituted-C₃heterocycloalkyl, oxo-substituted-C₄heterocycloalkyl, oxo-substituted-C₅heterocycloalkyl, oxo-substituted-C₆heterocycloalkyl, including aziridinonyl, azetidinonyl, pyrrolidinonyl, dioxolanonyl, imidazolidinonyl, pyrazolidinonyl, piperazinonyl, piperidinonyl, dioxanonyl, dithianonyl, thiomorpholinonyl, oxazepanonyl, oxiranonyl, oxetanonyl, quinuclidinonyl, tetrahydrofuranonyl, tetrahydropyranonyl, piperazinonyl, and the like. Thus, in some embodiments, R⁵ is azetidin-2-one-1-yl.

In some embodiments, R⁵ in Formula III is —C₃-C₆cycloalkyl, for example —C₃cycloalkyl, —C₄cycloalkyl, —C₅cycloalkyl, —C₆cycloalkyl, and the like. In some embodiments, R⁵ is —C₃cycloalkyl. Thus, in some embodiments, R⁵ is cyclopropyl.

In other embodiments, R⁵ in Formula III is —C(O)R⁹. In some embodiments wherein R⁹ is C₁-C₆alkyl, R⁵ is —C(O)C₁-C₆alkyl. Thus, in some embodiments wherein R⁹ is methyl, R⁵ is acetyl (i.e., —C(O)CH₃). In some embodiments wherein R⁹ is C₀-C₆alk-C₃-C₆cycloalkyl, R⁵ is —C(O)C₀-C₆alk-C₃-C₆cycloalkyl. In some embodiments wherein R⁹ is H, R⁵ is —CHO.

In some embodiments, R⁵ in Formula III is —CR⁸R^{8′}CN. Thus, in some embodiments wherein R⁸ and R^{8′} are both H, R⁵ in Formula III is cyanomethyl (i.e., —CH₂CN). In some embodiments wherein R⁸ is —C₁-C₆alkyl and R^{8′} is H, R⁵ is —CH(—C₁-C₆alkyl)CN. In some embodiments wherein R⁸ and R^{8′} are both —C₁-C₆alkyl, R⁵ is —C(C₁-C₆alkyl)(C₁-C₆alkyl)CN. In some embodiments wherein R⁸ is —C₀-C₆alk-OC₁-C₆alkyl and R^{8′} is H, R⁵ is —CH(—C₀-C₆alk-OC₁-C₆alkyl)CN. In some embodiments wherein R⁸ and R^{8′} are both —C₀-C₆alk-OC₁-C₆alkyl, R⁵ is —C(—C₀-C₆alk-OC₁-C₆alkyl)(—C₀-C₆alk-OC₁-C₆alkyl)CN.

In some embodiments, R⁵ in Formula III is CH₂NR⁸R^{8′}. Thus, in some embodiments wherein R⁸ and R^{8′} are both H, R⁵ in Formula III is aminomethyl (i.e., —CH₂NH₂). In some embodiments wherein R⁸ is —C₁-C₆alkyl and R^{8′} is H, R⁵ is —CH₂NH(C₁-C₆alkyl). In some embodiments wherein R⁸ and R^{8′} are both —C₁-C₆alkyl, R⁵ is —CH₂NH(C₁-C₆alkyl)(C₁-C₆alkyl). In some embodiments wherein R⁸ is —C₀-C₆alk-OC₁-C₆alkyl and R^{8′} is H, R⁵ is —CH₂NH(—C₀-C₆alk-OC₁-C₆alkyl). In some embodiments wherein R⁸ and R^{8′} are both —C₀-C₆alk-OC₁-C₆alkyl, R⁵ is —CH₂NH(—C₀-C₆alk-OC₁-C₆alkyl)(—C₀-C₆alk-OC₁-C₆alkyl).

In some embodiments, R⁵ in Formula III is —C₀-C₆alk-OH, for example, —C₀alk-OH, —C₁alk-OH, —C₂alk-OH, —C₃alk-OH, —C₄alk-OH, —C₅alk-OH, —C₆alk-OH, and the like. In some embodiments R⁵ is —C₁alk-OH. In some embodiments, R⁵ is hydroxymethyl (i.e., CH₂OH).

In some embodiments, R⁵ in Formula III is —NR⁸R^{8′}. Thus, in some embodiments wherein R⁸ and R^{8′} are both H, R⁵ is amino (i.e., —NH₂). In some embodiments wherein R⁸ is —C₁-C₆alkyl and R^{8′} is H, R⁵ is —NH(C₁-C₆alkyl). Thus, in some embodiments wherein R⁸ is methyl and R^{8′} is H, R⁵ is methylamino (i.e., —NHCH₃). In some embodiments wherein R⁸ and R^{8′} are both —C₁-C₆alkyl, R⁵ is —N(—C₁-C₆alkyl)(—C₁-C₆alkyl). In some embodiments wherein R⁸ is —C₀-C₆alk-OC₁-C₆alkyl and R^{8′} is H, R⁵ is —NH(—C₀-C₆alk-OC₁-C₆alkyl). In some embodiments wherein R⁸ and R^{8′} are both —C₀-C₆alk-OC₁-C₆alkyl, R⁵ is —N(—C₀-C₆alk-OC₁-C₆alkyl)(—C₀-C₆alk-OC₁-C₆alkyl).

In some embodiments, R⁵ in Formula III is —N(R⁹)CN. In some embodiments wherein R⁹ is —C₁-C₆alkyl, R⁵ is —N(C₁-C₆alkyl)CN. Thus, in some embodiments wherein R⁹ is methyl, R⁵ is —N(CH₃)CN. In some embodiments wherein R⁹ is —C₀-C₆alk-C₃-C₆cycloalkyl, R⁵ is —N(—C₀-C₆alk-C₃-C₆cycloalkyl)CN. In some embodiments wherein R⁹ is H, R⁵ is —NH—CN.

In some embodiments, R⁵ in Formula III is —O—C₁-C₄ alkyl, for example —O—C₁alkyl, —O—C₂alkyl, —O—C₃alkyl, and —O—C₄alkyl.

In some embodiments, R⁵ in Formula III is —NR⁹C(O)NR⁸R^{8′}. In some embodiments wherein R⁹ is H, R⁵ in Formula III is —NHC(O)NR⁸R^{8′}. In some embodiments wherein R⁹ is —C₁-C₆alkyl, R⁵ is —N(—C₁-C₆alkyl)C(O)NR⁸R^{8′}. In some embodiments wherein R⁹ is C₀-C₆alk-C₃-C₆cycloalkyl, R⁵ is —N(C₀-C₆alk-C₃-C₆cycloalkyl)C(O)NR⁸R^{8′}. In some embodiments wherein R⁸ is H, R⁵ is —NR⁹C(O)NHR^{8′}. In some embodiments wherein R⁸ is H and R^{8′} is H, R⁵ is —NR⁹C(O)NH₂. Thus, in some embodiments wherein R⁹ is H and R⁸ and R^{8′} are both H, R⁵ is urea-1-yl (i.e., —NHC(O)NH₂). In some embodiments wherein R⁹ is —C₁-C₆alkyl and R^{8′} is H, R⁵ is —NR⁹C(O)NH(C₁-C₆alkyl). In some embodiments wherein R⁸ and R^{8′} are both —C₁-C₆alkyl, R⁵ is —NR⁹C(O)N(C₁-C₆alkyl)(C₁-C₆alkyl). In some embodiments wherein R⁸ is —C₀-C₆alk-O—C₁-C₆alkyl and R^{8′} is H, R⁵ is —NR⁹C(O)NH(C₀-C₆alk-O—C₁-C₆alkyl). In some embodiments wherein R⁸ and R^{8′} are both —C₀-C₆alk-O—C₁-C₆alkyl, R⁵ is —NR⁹C(O)N(C₀-C₆alk-O—C₁-C₆alkyl)(C₀-C₆alk-O—C₁-C₆alkyl).

In some embodiments, R⁵ in Formula III is —OC(O)NR⁸R^{8′}. In some embodiments wherein R⁸ is H, R⁵ is —OC(O)NHR^{8′}. In some embodiments wherein R⁸ is H and R^{8′} is H, R⁵ is —OC(O)NH₂. In some embodiments wherein R⁸ is —C₁-C₆alkyl and R^{8′} is H, R⁵ is —OC(O)NH(C₁-C₆alkyl). In some embodiments wherein R⁸ and R^{8′} are both —C₁-C₆alkyl, R⁵ is —OC(O)N(C₁-C₆alkyl)(C₁-C₆alkyl). In some embodiments wherein R⁸ is —C₀-C₆alk-O—C₁-C₆alkyl and R^{8′} is H, R⁵ is —OC(O)NH(C₀-C₆alk-O—C₁-C₆alkyl). In some embodiments wherein R⁸ and R^{8′} are both —C₀-C₆alk-O—C₁-C₆alkyl, R⁵ is —OC(O)N(C₀-C₆alk-O—C₁-C₆alkyl)(C₀-C₆alk-O—C₁-C₆alkyl).

In some embodiments, R⁵ in Formula III is —NR⁹C(O)OR^{9a}. In some embodiments wherein R⁹ is H, R⁵ is —NHC(O)OR^{9a}. In some embodiments wherein R⁹ is —C₁-C₆alkyl, R⁵ is —N(—C₁-C₆alkyl)C(O)OR^{9a}. In some embodiments wherein R⁹ is C₀-C₆alk-C₃-C₆cycloalkyl, R⁵ is —N C₀-C₆alk-C₃-C₆cycloalkyl)C(O)OR^{9a}. In some embodiments wherein R^{9a} is —C₁-C₆alkyl, R⁵ is —NR⁹C(O)O—C₁-C₆alkyl. In some embodiments wherein R^{9a} is —C₀-C₆alk-C₃-C₆cycloalkyl, R⁵ is —NR⁹C(O)O—C₀-C₆alk-C₃-C₆cycloalkyl. In some embodiments wherein R⁹ is H and R^{9a} is —C₁-C₆alkyl, R⁵ is —NHC(O)O—C₁-C₆alkyl. Thus, in some embodiments wherein R⁹ is H and R^{9a} is methyl, R⁵ is —NHC(O)OCH₃.

In some aspects of Formula III, R⁸ and R^{8′} are each independently H, C₁-C₆alkyl, or —C₀-C₆alk-OC₁-C₆alkyl, or R⁸ and R^{8′}, together with the atom to which they are attached, form a C₃-C₆cycloalkyl ring or a C₂-C₆heterocycloalkyl ring.

In some aspects of Formula III, R⁸ and R^{8′} are each independently H, C₁-C₆alkyl (e.g., methyl, ethyl, propyl, isopropyl, butyl, isobutyl, s-butyl, t-butyl, pentyl, and the like), or —C₀-C₆alk-OC₁-C₆alkyl, (for example, —C₀alk-O—C₁alkyl, —C₁alk-O—C₁alkyl, —C₂alk-O—C₁alkyl, —C₃alk-O—C₁alkyl, —C₄alk-O—C₁alkyl, —C₅alk-O—C₁alkyl, —C₀alk-O—C₁alkyl, —C₀alk-O—C₂alkyl, —C₁alk-O—C₂alkyl, —C₂alk-O—C₂alkyl, —C₃alk-O—C₂alkyl, —C₄alk-O—C₂alkyl, —C₅alk-O—C₂alkyl, —C₆alk-O—C₂alkyl, —C₀alk-O—C₃alkyl, —C₁alk-O—C₃alkyl, —C₂alk-O—C₃alkyl, —C₃alk-O—C₃alkyl, —C₄alk-O—C₃alkyl, —C₅alk-O—C₃alkyl, —C₆alk-O—C₃alkyl, —C₀alk-O—C₄alkyl, —C₁alk-O—C₄alkyl, —C₂alk-O—C₄alkyl, —C₃alk-O—C₄alkyl, —C₄alk-O—C₄alkyl, —C₅alk-O—C₄alkyl, —C₆alk-O—C₄alkyl, —C₀alk-O—C₅alkyl, —C₁alk-O—C₅alkyl, —C₂alk-O—C₅alkyl, —C₃alk-O—C₅alkyl, —C₄alk-O—C₅alkyl, —C₅alk-O—C₅alkyl, —C₆alk-O—C₅alkyl, —C₀alk-O—C₆alkyl, —C₁alk-O—C₆alkyl, —C₂alk-O—C₆alkyl, —C₃alk-O—C₆alkyl, —C₄alk-O—C₆alkyl, —C₅alk-O—C₆alkyl, and —C₆alk-O—C₆alkyl). In some embodiments, R⁸ is C₁-C₆alkyl or —C₀-C₆alk-OC₁-C₆alkyl and R^{8′} is H, C₁-C₆alkyl, or —C₀-C₆alk-OC₁-C₆alkyl.

In some embodiments of Formula III, R⁸ is H or C₁-C₆alkyl. In some embodiments, R^{8′} is H or C₁-C₆alkyl. In some embodiments, R⁸ and R^{8′} are each H. In other embodiments, R⁸ and R^{8′} are each independently C₁-C₆alkyl. In some aspects, R⁸ is C₁-C₆alkyl and R^{8′} is H.

In other aspects of Formula III, R⁸ and R^{8′} are each independently —C₀-C₆alk-OC₁-C₆alkyl.

In other aspects of Formula III, R⁸ is —C₀-C₆alk-OC₁-C₆alkyl and R^{8′} is H.

In yet other aspects of Formula III, R⁸ and R^{8′}, together with the atom to which they are attached, form a C₂-C₆cycloalkyl ring, for example, cyclopropyl, cyclobutyl, cyclopentyl, or cyclohexyl, and the like, or a C₂-C₆heterocycloalkyl ring, for example, aziridinyl, azetidinyl, pyrrolidinyl, piperidinyl, and the like.

In some aspects of Formula III, R⁹ is H, —C₁-C₆alkyl (e.g., methyl, ethyl, propyl, isopropyl, butyl, isobutyl, s-butyl, t-butyl, pentyl, and the like), or C₀-C₆alk-C₃-C₆cycloalkyl (e.g., —C₀alk-C₃cycloalkyl, —C₁alk-C₃cycloalkyl, —C₂alk-C₃cycloalkyl, —C₃alk-C₃cycloalkyl, —C₄alk-C₃cycloalkyl, —C₅alk-C₃cycloalkyl, —C₆alk-C₃cycloalkyl, —C₀alk-C₄cycloalkyl, —C₁alk-C₄cycloalkyl, —C₂alk-C₄cycloalkyl, —C₃alk-C₄cycloalkyl, —C₄alk-C₄cycloalkyl, —C₅alk-C₄cycloalkyl, —C₆alk-C₄cycloalkyl, —C₀alk C₅cycloalkyl, —C₁alk-C₅cycloalkyl, —C₂alk-C₅cycloalkyl, —C₃alk-C₅cycloalkyl, —C₄alk-C₅cycloalkyl, —C₅alk-C₅cycloalkyl, —C₆alk-C₅cycloalkyl, —C₀alk-C₆cycloalkyl, —C₁alk-C₆cycloalkyl, —C₂alk-C₆cycloalkyl, —C₃alk-C₆cycloalkyl, —C₄alk-C₆cycloalkyl, —C₅alk-C₆cycloalkyl, and —C₆alk-C₆cycloalkyl).

In some aspects of Formula III, R^{9a} is —C₁-C₆alkyl, or C₀-C₆alk-C₃-C₆cycloalkyl.

In some embodiments, R^{9a} in Formula III is —C₁-C₆alkyl (e.g., methyl, ethyl, propyl, isopropyl, butyl, isobutyl, s-butyl, t-butyl, pentyl, and the like), or C₀-C₆alk-C₃-C₆cycloalkyl (e.g., —C₀alk-C₃cycloalkyl, —C₁alk-C₃cycloalkyl, —C₂alk-C₃cycloalkyl, —C₃alk-C₃cycloalkyl, —C₄alk-C₃cycloalkyl, —C₅alk-C₃cycloalkyl, —C₆alk-C₃cycloalkyl, —C₀alk-C₄cycloalkyl, —C₁alk-C₄cycloalkyl, —C₂alk-C₄cycloalkyl, —C₃alk-C₄cycloalkyl, —C₄alk-C₄cycloalkyl, —C₅alk-C₄cycloalkyl, —C₆alk-C₄cycloalkyl, —C₀alk-C₅cycloalkyl, —C₁alk-C₅cycloalkyl, —C₂alk-C₅cycloalkyl, —C₃alk-C₅cycloalkyl, —C₄alk-C₅cycloalkyl, —C₅alk-C₅cycloalkyl, —C₆alk-C₅cycloalkyl, —C₀alk-C₆cycloalkyl, —C₁alk-C₆cycloalkyl, —C₂alk-C₆cycloalkyl, —C₃alk-C₆cycloalkyl, —C₄alk-C₆cycloalkyl, —C₅alk-C₆cycloalkyl, and —C₆alk-C₆cycloalkyl).

In some aspects of Formula III and Formula IV, R⁶ is C₁-C₆alkyl or C₀-C₆alk-C₃-C₆cycloalkyl. In some embodiments, R⁶ is —C₁-C₆alkyl, for example, methyl, ethyl, propyl, isopropyl, butyl, isobutyl, s-butyl, t-butyl, pentyl, and the like. Thus, in some embodiments, R⁶ is methyl (i.e., —CH₃, or Me). In other embodiments, R⁶ is —C₀-C₆alk-C₃-C₆cycloalkyl, for example, —C₀alk-C₃cycloalkyl, —C₁alk-C₃cycloalkyl, —C₂alk-C₃cycloalkyl, —C₃alk-C₃cycloalkyl, —C₄alk-C₃cycloalkyl, —C₅alk-C₃cycloalkyl, —C₆alk-C₃cycloalkyl, —C₀alk-C₄cycloalkyl, —C₁alk-C₄cycloalkyl, —C₂alk-C₄cycloalkyl, —C₃alk-C₄cycloalkyl, —C₄alk-C₄cycloalkyl, —C₅alk-C₄cycloalkyl, —C₆alk-C₄cycloalkyl, —C₀alk-C₅cycloalkyl, —C₁alk-C₅cycloalkyl, —C₂alk-C₅cycloalkyl, —C₃alk-C₅cycloalkyl, —C₄alk-C₅cycloalkyl, —C₅alk-C₅cycloalkyl, —C₀alk-C₅cycloalkyl, —C₀alk-C₆cycloalkyl, —C₁alk-C₆cycloalkyl, —C₂alk-C₆cycloalkyl, —C₃alk-C₆cycloalkyl, —C₄alk-C₆cycloalkyl, —C₅alk-C₆cycloalkyl, —C₆alk-C₆cycloalkyl,

In some aspects of Formula III and Formula IV, R¹¹ is H, —C₁-C₆alkyl, —C₁-C₆haloalkyl, —C₀-C₆alk-C₃-C₆cycloalkyl, —C₀-C₆alk-C₃-C₆halocycloalkyl, —C₀-C₆alk-OH, —C₀-C₆alk-NH₂, —C₀-C₆alk-NH—C₁-C₆alkyl, —C₀-C₆alk-N(C₁-C₆alkyl)-C₁-C₆alkyl, —C₀-C₆alk-NH—C₃-C₆cycloalkyl, —C₀-C₆alk-N(C₁-C₆alkyl)-C₃-C₆cycloalkyl; or R¹¹ and R¹, together with the atom to which they are attached, form a C₃-C₆cycloalkyl ring or a heterocycloalkyl ring.

In some embodiments, R¹¹ in Formula III and Formula IV is H.

In some embodiments, R¹¹ in Formula III and Formula IV is —C₁-C₆alkyl, for example, methyl, ethyl, propyl, isopropyl, butyl, isobutyl, s-butyl, t-butyl, pentyl, and the like.

In some embodiments, R¹¹ in Formula III and Formula IV is —C₁-C₆haloalkyl, for example, C₁haloalkyl, C₂haloalkyl, C₃haloalkyl, C₄haloalkyl, C₅haloalkyl, or C₆haloalkyl.

In some aspects, R¹¹ in Formula III and Formula IV is —C₀-C₆alk-C₃-C₆cycloalkyl, for example —C₀alk-C₃cycloalkyl, —C₁alk-C₃cycloalkyl, —C₂alk-C₃cycloalkyl, —C₃alk-C₃cycloalkyl, —C₄alk-C₃cycloalkyl, —C₅alk-C₃cycloalkyl, —C₆alk-C₃cycloalkyl, —C₀alk-C₄cycloalkyl, —C₁alk-C₄cycloalkyl, —C₂alk-C₄cycloalkyl, —C₃alk-C₄cycloalkyl, —C₄alk-C₄cycloalkyl, —C₅alk-C₄cycloalkyl, —C₆alk-C₄cycloalkyl, —C₀alk-C₅cycloalkyl, —C₁alk-C₅cycloalkyl, —C₂alk-C₅cycloalkyl, —C₃alk-C₅cycloalkyl, —C₄alk-C₅cycloalkyl, —C₅alk-C₅cycloalkyl, —C₆alk-C₅cycloalkyl, —C₀alk-C₆cycloalkyl, —C₁alk-C₆cycloalkyl, —C₂alk-C₆cycloalkyl, —C₃alk-C₆cycloalkyl, —C₄alk-C₆cycloalkyl, —C₅alk-C₆cycloalkyl, and —C₀alk-C₆cycloalkyl. In some embodiments, R¹¹ is —C₁alk-C₃cycloalkyl. Thus, in some embodiments, R¹¹ is —CH₂-cyclopropyl.

In some aspects, R¹¹ in Formula III and Formula IV is —C₀-C₆alk-C₃-C₆halocycloalkyl, for example —C₀alk-C₃halocycloalkyl, —C₁alk-C₃halocycloalkyl, —C₂alk-C₃halocycloalkyl, —C₃alk-C₃halocycloalkyl, —C₄alk-C₃halocycloalkyl, —C₅alk-C₃halocycloalkyl, —C₆alk-C₃halocycloalkyl, —C₀alk-C₄halocycloalkyl, —C₁alk-C₄halocycloalkyl, —C₂alk-C₄halocycloalkyl, —C₃alk-C₄halocycloalkyl, —C₄alk-C₄halocycloalkyl, —C₅alk-C₄halocycloalkyl, —C₆alk-C₄halocycloalkyl, —C₀alk-C₅halocycloalkyl, —C₁alk-C₅halocycloalkyl, —C₂alk-C₅halocycloalkyl, —C₃alk-C₅halocycloalkyl, —C₄alk-C₅halocycloalkyl, —C₅alk-C₅halocycloalkyl, —C₆alk-C₅halocycloalkyl, —C₀alk-C₆halocycloalkyl, —C₁alk-C₆halocycloalkyl, —C₂alk-C₆halocycloalkyl, —C₃alk-C₆halocycloalkyl, —C₄alk-C₆halocycloalkyl, —C₅alk-C₆halocycloalkyl, and —C₀alk-C₆halocycloalkyl.

In some aspects, R¹¹ in Formula III and Formula IV is —C₀-C₆alk-OH, for example, —C₀alk-OH (i.e., —OH), —C₁alk-OH, —C₂alk-OH, —C₃alk-OH, —C₄alk-OH, —C₅alk-OH, —C₆alk-OH, and the like. In some embodiments, R¹¹ is —C₁alk-OH. Thus, in some embodiments, R¹¹ is hydroxymethyl (i.e., —CH₂OH).

In some aspects, R¹¹ in Formula III and Formula IV is —C₀-C₆alk-NH₂, for example, —C₀alk-NH₂ (i.e., —NH₂), —C₁alk-NH₂, —C₂alk-NH₂, —C₃alk-NH₂, —C₄alk-NH₂, —C₅alk-NH₂, —C₆alk-NH₂, and the like. In some embodiments, R¹¹ is —C₁alk-NH₂. Thus, in some embodiments, R¹¹ is aminomethyl (i.e., —CH₂NH₂).

In some aspects, R¹¹ in Formula III and Formula IV is —C₀-C₆alk-NH—C₁-C₆alkyl, for example, —C₀alk-NH—C₁alkyl, —C₁alk-NH—C₁alkyl, —C₂alk-NH—C₁alkyl, —C₃alk-NH—C₁alkyl, —C₄alk-NH—C₁alkyl, —C₅alk-NH—C₁alkyl, —C₆alk-NH—C₁alkyl, —C₀alk-NH—C₂alkyl, —C₁alk-NH—C₂alkyl, —C₂alk-NH—C₂alkyl, —C₃alk-NH—C₂alkyl, —C₄alk-NH—C₂alkyl, —C₅alk-NH—C₂alkyl, —C₆alk-NH—C₂alkyl, —C₀alk-NH—C₃alkyl, —C₁alk-NH—C₃alkyl, —C₂alk-NH—C₃alkyl, —C₃alk-NH—C₃alkyl, —C₄alk-NH—C₃alkyl, —C₅alk-NH—C₃alkyl, —C₆alk-NH—C₃alkyl, —C₀alk-NH—C₄alkyl, —C₁alk-NH—C₄alkyl, —C₂alk-NH—C₄alkyl, —C₃alk-NH—C₄alkyl, —C₄alk-NH—C₄alkyl, —C₅alk-NH—C₄alkyl, —C₆alk-NH—C₄alkyl, —C₀alk-NH—C₅alkyl, —C₁alk-NH—C₅alkyl, —C₂alk-NH—C₅alkyl, —C₃alk-NH—C₅alkyl, —C₄alk-NH—C₅alkyl, —C₅alk-NH—C₅alkyl, —C₆alk-NH—C₅alkyl, —C₀alk-NH—C₆alkyl, —C₁alk-NH—C₆alkyl, —C₂alk-NH—C₆alkyl, —C₃alk-NH—C₆alkyl, —C₄alk-NH—C₆alkyl, —C₅alk-NH—C₆alkyl, and —C₆alk-NH—C₆alkyl.

In some aspects, R¹¹ in Formula III and Formula IV is —C₀-C₆alk-N(C₁-C₆alkyl)-C₁-C₆alkyl, for example, —C₀alk-N(C₁-C₆alkyl)-C₁alkyl, —C₁alk-N(C₁-C₆alkyl)-C₁alkyl, —C₂alk-N(C₁-C₆alkyl)-C₁alkyl, —C₃alk-N(C₁-C₆alkyl)-C₁alkyl, —C₄alk-N(C₁-C₆alkyl)-C₁alkyl, —C₅alk-N(C₁-C₆alkyl)-C₁alkyl, —C₀alk-N(C₁-C₆alkyl)-C₁alkyl, —C₀alk-N(C₁-C₆alkyl)-C₂alkyl, —C₁alk-N(C₁-C₆alkyl)-C₂alkyl, —C₂alk-N(C₁-C₆alkyl)-C₂alkyl, —C₃alk-N(C₁-C₆alkyl)-C₂alkyl, —C₄alk-N(C₁-C₆alkyl)-C₂alkyl, —C₅alk-N(C₁-C₆alkyl)-C₂alkyl, —C₆alk-N(C₁-C₆alkyl)-C₂alkyl, —C₀alk-N(C₁-C₆alkyl)-C₃alkyl, —C₁alk-N(C₁-C₆alkyl)-C₃alkyl, —C₂alk-N(C₁-C₆alkyl)-C₃alkyl, —C₃alk-N(C₁-C₆alkyl)-C₃alkyl, —C₄alk-N(C₁-C₆alkyl)-C₃alkyl, —C₅alk-N(C₁-C₆alkyl)-C₃alkyl, —C₆alk-N(C₁-C₆alkyl)-C₃alkyl, —C₀alk-N(C₁-C₆alkyl)-C₄alkyl, —C₁alk-N(C₁-C₆alkyl)-C₄alkyl, —C₂alk-N(C₁-C₆alkyl)-C₄alkyl, —C₃alk-N(C₁-C₆alkyl)-C₄alkyl, —C₄alk-N(C₁-C₆alkyl)-C₄alkyl, —C₅alk-N(C₁-C₆alkyl)-C₄alkyl, —C₆alk-N(C₁-C₆alkyl)-C₄alkyl, —C₀alk-N(C₁-C₆alkyl)-C₅alkyl, —C₁alk-N(C₁-C₆alkyl)-C₅alkyl, —C₂alk-N(C₁-C₆alkyl)-C₅alkyl, —C₃alk-N(C₁-C₆alkyl)-C₅alkyl, —C₄alk-N(C₁-C₆alkyl)-C₅alkyl, —C₅alk-N(C₁-C₆alkyl)-C₅alkyl, —C₆alk-N(C₁-C₆alkyl)-C₅alkyl, —C₀alk-N(C₁-C₆alkyl)-C₆alkyl, —C₁alk-N(C₁-C₆alkyl)-C₆alkyl, —C₂alk-N(C₁-C₆alkyl)-C₆alkyl, —C₃alk-N(C₁-C₆alkyl)-C₆alkyl, —C₄alk-N(C₁-C₆alkyl)-C₆alkyl, —C₅alk-N(C₁-C₆alkyl)-C₆alkyl, —C₆alk-N(C₁-C₆alkyl)-C₆alkyl and the like.

In some aspects, R¹¹ in Formula III and Formula IV is —C₀-C₆alk-NH—C₃-C₆cycloalkyl, for example, —C₀alk-NH—C₃cycloalkyl, —C₁alk-NH—C₃cycloalkyl, —C₂alk-NH—C₃cycloalkyl, —C₃alk-NH—C₃cycloalkyl, —C₄alk-NH—C₃cycloalkyl, —C₅alk-NH—C₃cycloalkyl, —C₆alk-NH—C₃cycloalkyl, —C₀alk-NH—C₄cycloalkyl, —C₁alk-NH—C₄cycloalkyl, —C₂alk-NH—C₄cycloalkyl, —C₃alk-NH—C₄cycloalkyl, —C₄alk-NH—C₄cycloalkyl, —C₅alk-NH—C₄cycloalkyl, —C₆alk-NH—C₄cycloalkyl, —C₀alk-NH—C₅cycloalkyl, —C₁alk-NH—C₅cycloalkyl, —C₂alk-NH—C₅cycloalkyl, —C₃alk-NH—C₅cycloalkyl, —C₄alk-NH—C₅cycloalkyl, —C₅alk-NH—C₅cycloalkyl, —C₆alk-NH—C₅cycloalkyl, —C₀alk-NH—C₆cycloalkyl, —C₁alk-NH—C₆cycloalkyl, —C₂alk-NH—C₆cycloalkyl, —C₃alk-NH—C₆cycloalkyl, —C₄alk-NH—C₆cycloalkyl, —C₅alk-NH—C₆cycloalkyl, —C₆alk-NH—C₆cycloalkyl, and the like.

In some aspects, R¹¹ in Formula III and Formula IV is —C₀-C₆alk-N(C₁-C₆alkyl)-C₃-C₆cycloalkyl, for example, —C₀alk-N(C₁-C₆alkyl)-C₃cycloalkyl, —C₁alk-N(C₁-C₆alkyl)-C₃cycloalkyl, —C₂alk-N(C₁-C₆alkyl)-C₃cycloalkyl, —C₃alk-N(C₁-C₆alkyl)-C₃cycloalkyl, —C₄alk-N(C₁-C₆alkyl)-C₃cycloalkyl, —C₅alk-N(C₁-C₆alkyl)-C₃cycloalkyl, —C₆alk-N(C₁-C₆alkyl)-C₃cycloalkyl, —C₀alk-N(C₁-C₆alkyl)-C₄cycloalkyl, —C₁alk-N(C₁-C₆alkyl)-C₄cycloalkyl, —C₂alk-N(C₁-C₆alkyl)-C₄cycloalkyl, —C₃alk-N(C₁-C₆alkyl)-C₄cycloalkyl, —C₄alk-N(C₁-C₆alkyl)-C₄cycloalkyl, —C₅alk-N(C₁-C₆alkyl)-C₄cycloalkyl, —C₆alk-N(C₁-C₆alkyl)-C₄cycloalkyl, —C₀alk-N(C₁-C₆alkyl)-C₅cycloalkyl, —C₁alk-N(C₁-C₆alkyl)-C₅cycloalkyl, —C₂alk-N(C₁-C₆alkyl)-C₅cycloalkyl, —C₃alk-N(C₁-C₆alkyl)-C₅cycloalkyl, —C₄alk-N(C₁-C₆alkyl)-C₅cycloalkyl, —C₅alk-N(C₁-C₆alkyl)-C₅cycloalkyl, —C₆alk-N(C₁-C₆alkyl)-C₅cycloalkyl, —C₀alk-N(C₁-C₆alkyl)-C₆cycloalkyl, —C₁alk-N(C₁-C₆alkyl)-C₆cycloalkyl, —C₂alk-N(C₁-C₆alkyl)-C₆cycloalkyl, —C₃alk-N(C₁-C₆alkyl)-C₆cycloalkyl, —C₄alk-N(C₁-C₆alkyl)-C₆cycloalkyl, —C₅alk-N(C₁-C₆alkyl)-C₆cycloalkyl, —C₆alk-N(C₁-C₆alkyl)-C₆cycloalkyl, and the like.

In some aspects of Formula III and Formula IV, R¹¹ and R¹, together with the atom to which they are attached, form a C₃-C₆cycloalkyl ring or a heterocycloalkyl ring.

In some aspects of Formula III, R¹⁰ is -halo or —C₁-C₆alkyl. Thus in some embodiments of the compound of Formula III, R¹⁰ is halo (e.g., —F, —Cl, —Br, or —I). Thus, in some embodiments of the compound of Formula III, R¹⁰ is —F. In other embodiments of the compound of Formula III, R¹⁰ is —Cl.

In other aspects of Formula III, R¹⁰ is —C₁-C₆alkyl (e.g., methyl, ethyl, propyl, isopropyl, butyl, isobutyl, s-butyl, t-butyl, pentyl, and the like). Thus in some embodiments of Formula III, R¹⁰ is methyl (i.e., —CH₃, or Me).

In some aspects of Formula IV, R^{10a} is H, halo, or —C₁-C₆alkyl. Thus in some embodiments of the compounds of Formula IV, R^{10a} is H. In other embodiments of the compound of Formula IV, R^{10a} is halo, for example, —F, —Cl, —Br, or —I. Thus, in some embodiments, R^{10a} is —F. In yet other embodiments of the compound of Formula IV, R^{10a} is —C₁. In other embodiments of the compounds of Formula IV, R^{10a} is —C₁-C₆alkyl, for example, methyl, ethyl, propyl, isopropyl, butyl, isobutyl, s-butyl, t-butyl, pentyl, and the like. Thus, in some embodiments, R^{10a} is methyl (i.e., —CH₃, or Me).

In preferred embodiments of the compounds of Formula III or Formula IV, R¹ is —C₀-C₆alk-C₁-C₆alkyl, —C₀-C₆alk-C₁-C₆haloalkyl, —C₀-C₆alk-C≡CH, —C₀-C₆alk-C≡C—C₁-C₆alkyl, —C₀-C₆alk-C≡C—C₁-C₆haloalkyl, —C₀-C₆alk-C≡C—C₃-C₆cycloalkyl, —C₁-C₆alk-aryl, —C₀-C₆alk-S-aryl, —C₀-C₆alk-S(O)-aryl, —C₀-C₆alk-S(O)₂-aryl, or —C₀-C₆alk-O-aryl.

More preferred embodiments of the compounds of Formula III or Formula IV are those wherein R¹ is —CH(OH)—C₁-C₆alkyl, —CH(F)—C₁-C₆alkyl, —CH(NH₂)—C₁-C₆alkyl, —CH(Me)-C₁-C₆alkyl, —C(Me)(OH)—C₁-C₆alkyl, —CH(OH)—C₁-C₆ haloalkyl, —CH(F)—C₁-C₆ haloalkyl, —CH(NH₂)—C₁-C₆ haloalkyl, —CH(Me)-C₁-C₆ haloalkyl, —C(Me)(OH)—C₁-C₆ haloalkyl, —CH(OH)—C≡CH, —CH(F)—C≡CH, —CH(NH₂)—C≡CH, —CH(Me)-C≡CH, —C(Me)(OH)—C≡CH, —CH(OH)—C≡C—C₁-C₆alkyl, —CH(F)—C≡C—C₁-C₆alkyl, —CH(NH₂)—C≡C—C₁-C₆alkyl, —CH(Me)-C≡C—C₁-C₆alkyl, —C(Me)(OH)—C≡C—C₁-C₆alkyl, —CH(OH)—C≡C—C₁-C₆haloalkyl, —CH(F)—C≡C—C₁-C₆haloalkyl, —CH(NH₂)—C≡C—C₁-C₆haloalkyl, —CH(Me)-C≡C— C₁-C₆haloalkyl, —C(Me)(OH)—C≡C—C₁-C₆haloalkyl, —CH(OH)—C≡C—C₃-C₆cycloalkyl, —CH(F)—C≡C—C₃-C₆cycloalkyl, —CH(NH₂)—C≡C—C₃-C₆cycloalkyl, —CH(Me)-C≡C—C₃-C₆cycloalkyl, —C(Me)(OH)—C≡C—C₃-C₆cycloalkyl, —CH₂-aryl, —CH(OH)-aryl, —CH(F)-aryl, —CH(NH₂)-aryl, —CH(Me)-aryl, —C(Me)(OH)-aryl, —S-aryl, —S(O)-aryl, —S(O)₂-aryl, or —O-aryl.

Most preferred embodiments of the compounds of Formula III or Formula IV are those wherein R¹ is —CH(OH)—C≡C—CH₃, —CH(F)—C≡C—CH₃, —CH(NH₂)—C≡C—CH₃, —CH(Me)-C≡C—CH₃, or —C(Me)(OH)—C≡C—CH₃, —CH(OH)—C≡C—CH₃, —CH(OH)—C≡C—CF₃, —CH(F)—C≡C—CF₃, —CH(NH₂)—C≡C—CF₃, —CH(Me)-C≡C—CF₃, or —C(Me)(OH)—C≡C—CF₃, —CH(OH)—C≡C-cyclopropyl, —CH(F)—C≡C-cyclopropyl, —CH(NH₂)—C≡C-cyclopropyl, —CH(Me)-C≡C-cyclopropyl, or —C(Me)(OH)—C—C-cyclopropyl, —CH₂-4-chlorophenyl, —CH₂-3,4-dichlorophenyl, —CH₂-3,4-difluorophenyl, —CH₂-3-fluoro-4-chlorophenyl, —CH₂-3-chloro-4-fluorophenyl, —CH(OH)-4-chlorophenyl, —CH(OH)-3,4-dichlorophenyl, —CH(OH)-3,4-difluorophenyl, —CH(OH)-3-fluoro-4-chlorophenyl, —CH(OH)-3-chloro-4-fluorophenyl, —CH(F)-aryl-4-chlorophenyl, —CH(F)-3,4-dichlorophenyl, —CH(F)-3,4-difluorophenyl, —CH(F)-3-fluoro-4-chlorophenyl, —CH(F)-3-chloro-4-fluorophenyl, —CH(NH₂)-aryl-4-chlorophenyl, —CH(NH₂)-3,4-dichlorophenyl, —CH(NH₂)-3,4-difluorophenyl, —CH(NH₂)-3-fluoro-4-chlorophenyl, —CH(NH₂)-3-chloro-4-fluorophenyl, —CH(Me)-aryl-4-chlorophenyl, —CH(Me)-3,4-dichlorophenyl, —CH(Me)-3,4-difluorophenyl, —CH(Me)-3-fluoro-4-chlorophenyl, —CH(Me)-3-chloro-4-fluorophenyl, —C(Me)(OH)-aryl-4-chlorophenyl, —C(Me)(OH)-3,4-dichlorophenyl, —C(Me)(OH)-3,4-difluorophenyl, —C(Me)(OH)-3-fluoro-4-chlorophenyl, or —C(Me)(OH)-3-chloro-4-fluorophenyl, —S-4-chlorophenyl, —S-3,4-dichlorophenyl, —S-3,4-difluorophenyl, —S-3-fluoro-4-chlorophenyl, or —S-3-chloro-4-fluorophenyl, —S(O)-4-chlorophenyl, —S(O)-3,4-dichlorophenyl, —S(O)-3,4-difluorophenyl, —S(O)-3-fluoro-4-chlorophenyl, or —S(O)-3-chloro-4-fluorophenyl, —S(O)₂-4-chlorophenyl, —S(O)₂-3,4-dichlorophenyl, —S(O)₂-3,4-difluorophenyl, —S(O)₂-3-fluoro-4-chlorophenyl, or —S(O)₂-3-chloro-4-fluorophenyl, —O-4-chlorophenyl, —O-3,4-dichlorophenyl, —O-3,4-difluorophenyl, —O-3-fluoro-4-chlorophenyl, or —O-3-chloro-4-fluorophenyl.

In some preferred embodiments, R¹ is ((cyclopropylmethyl)amino)quinolin-7-yl-ethyl, 2-(methylamino)quinolin-7-yl)ethyl, 2-(2-aminoquinolin-7-yl)ethyl, 2-(2-amino-3-chloroquinolin-7-yl)ethyl, R¹ is —CH(OH)-4-chlorophenyl, —CH(OH)-3,4-difluorophenyl, —CH(OH)-3,4-dichlorophenyl, or —C(Me)(OH)-3,4-dichlorophenyl.

Some aspects of the disclosure are directed to compounds of Formula IIID or IVD:

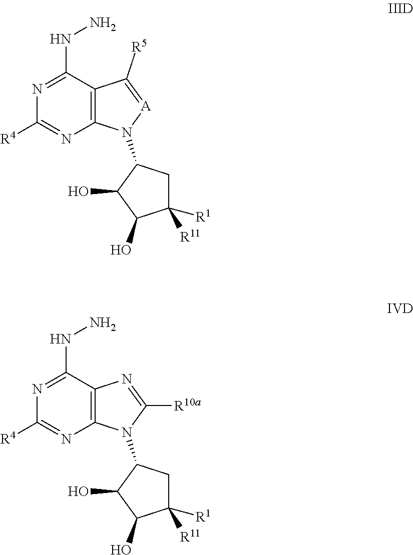

In some embodiments, the disclosure is directed to compounds of Formula IIID, wherein A is CH, R¹ is —C₁-C₆alk-aryl, R⁴ is H, R⁵ is H or F, and R¹¹ is H. In some embodiments, the compounds of Formula IIID are those wherein A is CH, R¹ is —CH(OH)-4-chlorophenyl, —CH(OH)-3,4-difluorophenyl, —CH(OH)-3,4-dichlorophenyl, or —C(Me)(OH)-3,4-dichlorophenyl, R⁴ is H, R⁵ is H or F, and R¹¹ is H.

Some aspects of the disclosure are directed to compounds of Formula IIIE or IVE:

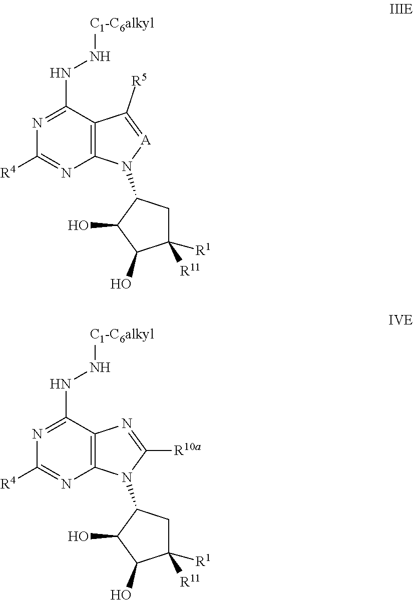

In some embodiments, the compounds of Formula IIIE are those wherein A is CH, R¹ is —C₀-C₆alk-heteroaryl, R⁴ is H, R⁵ is H, R¹¹ is H, and —C₁-C₆alkyl is methyl. In some embodiments, the compounds of Formula IIIE are those wherein A is CH, R¹ is ((cyclopropylmethyl)amino)quinolin-7-yl-ethyl, 2-(methylamino)quinolin-7-yl)ethyl, 2-(2-aminoquinolin-7-yl)ethyl, or 2-(2-amino-3-chloroquinolin-7-yl)ethyl; R⁴ is H, R⁵ is H, R¹¹ is H, and —C₁-C₆alkyl is methyl.

Some aspects of the disclosure are directed to compounds of Formula IIIF or IVF:

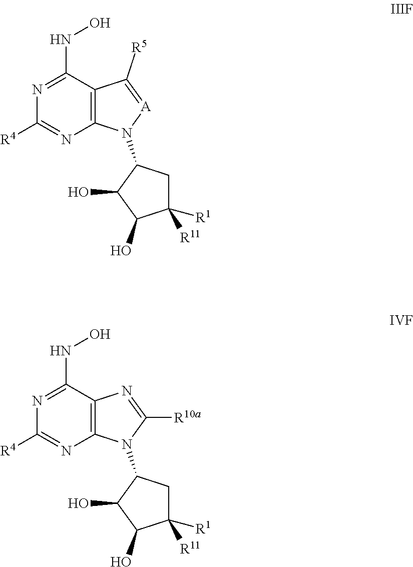

In some embodiments, the disclosure is directed to compounds of Formula IIIF, wherein A is CH, R¹ is —C₁-C₆alk-aryl or —C₀-C₆alk-heteroaryl, R⁴ is H, R⁵ is H, and R¹¹ is H. In some embodiments, the compounds of Formula IIIF are those wherein A is CH, R¹ is —C₁-C₆alk-aryl, R⁴ is H, R⁵ is H or F, and R¹¹ is H. In some embodiments, the compounds of Formula IIIF are those wherein A is CH, R¹ is —CH(OH)-4-chlorophenyl, —CH(OH)-3,4-difluorophenyl, —CH(OH)-3,4-dichlorophenyl, or —C(Me)(OH)-3,4-dichlorophenyl; R⁴ is H, R⁵ is H or F, and R¹¹ is H. In some embodiments, the compounds of Formula IIIF are those wherein A is CH, R¹ is —C₀-C₆alk-heteroaryl, R⁴ is H, R⁵ is H or F, and R¹¹ is H. In some embodiments, the compounds of Formula IIIF are those wherein A is CH, R¹ is ((cyclopropylmethyl)amino)quinolin-7-yl-ethyl, 2-(methylamino)quinolin-7-yl)ethyl, 2-(2-aminoquinolin-7-yl)ethyl, or 2-(2-amino-3-chloroquinolin-7-yl)ethyl, R⁴ is H, R⁵ is H or F, and R¹¹ is H.

Some aspects of the disclosure are directed to compounds of Formula IIIG or IVG:

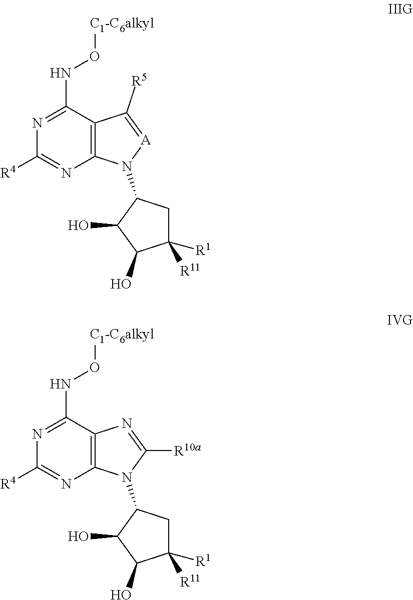

In some embodiments, the disclosure is directed to compounds of Formula IIIG, wherein A is CH, R¹ is —C₁-C₆alk-aryl or —C₀-C₆alk-heteroaryl, R⁴ is H, R⁵ is H, R¹¹ is H, and —C₁-C₆alkyl is methyl. In some embodiments, the compounds of Formula IIIG are those wherein A is CH, R¹ is —C₁-C₆alk-aryl, R⁴ is H, R⁵ is H, R¹¹ is H, and —C₁-C₆alkyl is methyl. In some embodiments, the compounds of Formula IIIG are those wherein A is CH, R¹ is —CH(OH)-4-chlorophenyl, —CH(OH)-3,4-difluorophenyl, —CH(OH)-3,4-dichlorophenyl, or —C(Me)(OH)-3,4-dichlorophenyl; R⁴ is H, R⁵ is H, R¹¹ is H, and —C₁-C₆alkyl is methyl. In some embodiments, the compounds of Formula IIIG are those wherein A is CH, R¹ is —C₀-C₆alk-heteroaryl, R⁴ is H, R⁵ is H, R¹¹ is H, and —C₁-C₆alkyl is methyl. In some embodiments, the compounds of Formula IIIG are those wherein A is CH, R¹ is ((cyclopropylmethyl)amino)quinolin-7-yl-ethyl, 2-(methylamino)quinolin-7-yl)ethyl, 2-(2-aminoquinolin-7-yl)ethyl, or 2-(2-amino-3-chloroquinolin-7-yl)ethyl; R⁴ is H, R⁵ is H, R¹¹ is H, and —C₁-C₆alkyl is methyl.

Some aspects of the disclosure are directed to compounds of Formula IIIH or IVH:

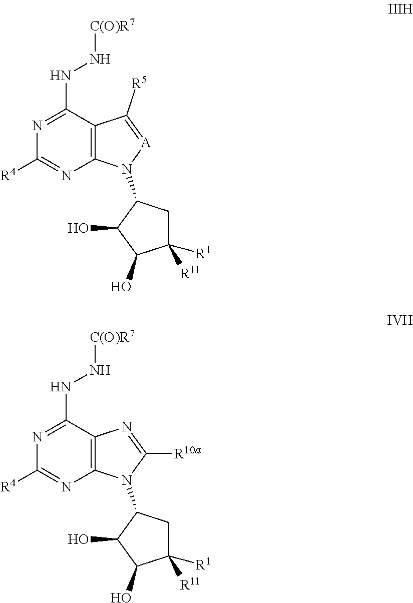

Some aspects of the disclosure are directed to compounds of Formula IIIB

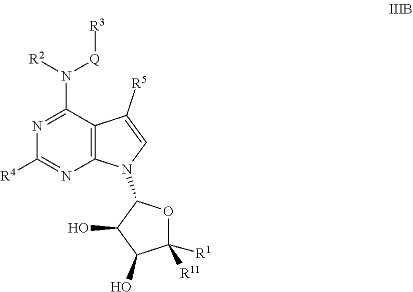

wherein Q is NH, R¹ is —C₁-C₆alk-aryl or —C₀-C₆alk-heteroaryl, R² is methyl, R³ is H, R⁴ is H, R⁵ is H, and R¹¹ is H. In some embodiments, the compounds of Formula IIIB are those wherein Q is NH, R¹ is —C₁-C₆alk-aryl, R² is methyl, R³ is H, R⁴ is H, R⁵ is H, and R¹¹ is H. In some embodiments, the compounds of Formula IIIB are those wherein Q is NH, R¹ is —CH(OH)-4-chlorophenyl, —CH(OH)-3,4-difluorophenyl, —CH(OH)-3,4-dichlorophenyl, or —C(Me)(OH)-3,4-dichlorophenyl; R² is methyl, R³ is H, R⁴ is H, R⁵ is H, and R¹¹ is H. In some embodiments, the compounds of Formula IIIB are those wherein Q is NH, R¹ is —C₀-C₆alk-heteroaryl, R² is methyl, R³ is H, R⁴ is H, R⁵ is H, and R¹¹ is H. In some embodiments, the compounds of Formula IIIB are those wherein Q is NH, R¹ is ((cyclopropylmethyl)amino)quinolin-7-yl-ethyl, 2-(methylamino)quinolin-7-yl)ethyl, 2-(2-aminoquinolin-7-yl)ethyl, or 2-(2-amino-3-chloroquinolin-7-yl)ethyl; R² is methyl, R³ is H, R⁴ is H, R⁵ is H, and R¹¹ is H.

Stereoisomers of compounds of Formula III or Formula IV are also contemplated.

Pharmaceutically acceptable salts and solvates of the compounds of Formula III or Formula IV are also within the scope of the disclosure.

The disclosure is also directed to compounds of Formula V or Formula VI, as well as pharmaceutically acceptable salts of compounds of Formula V and of Formula VI. In some aspects, the disclosure is directed to compounds and salts of Formula V:

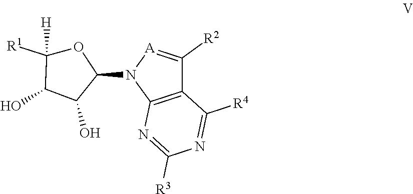

In other aspects, the disclosure is directed to compounds and salts of Formula VI:

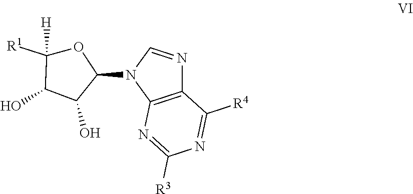

According to the disclosure, A in Formula V is N or CH. In some aspects, A is N and the compounds of Formula V are of Formula VA:

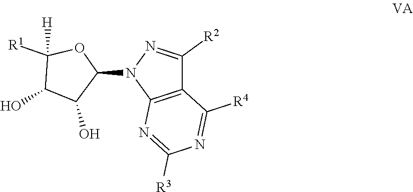

In some aspects, A is CH and the compounds of Formula V are of Formula VB:

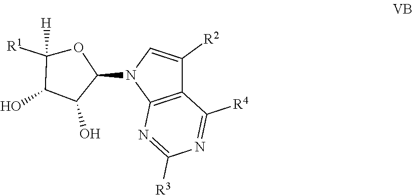

According to the disclosure, R¹ in Formula V or Formula VI is —C₁-C₆alk-aryl, —C₁-C₆alk-heteroaryl, —C₁-C₆alk-C≡CH, —C₁-C₆alk-C≡C—C₁-C₆alkyl, —C₁-C₆alk-C≡C—C₁-C₆haloalkyl, or —C₁-C₆alk-C≡C—C₃-C₆cycloalkyl.

In some aspects, R¹ in Formula V or Formula VI is —C₁-C₆alk-aryl, for example, —C₁alk-aryl, —C₂alk-aryl, —C₃alk-aryl, —C₄alk-aryl, —C₅alk-aryl, —C₆alk-aryl, —CH₂aryl, —CH(OH)-aryl, —CH(F)-aryl, —CH(NH₂)-aryl, —CH(Me)-aryl, —C(Me)(OH)-aryl, —CH(CH₂OH)-aryl and the like. In some embodiments the -aryl is -4-chlorophenyl, -3,4-dichlorophenyl, -3,4-difluorophenyl, -3-fluoro-4-chlorophenyl, 3-methyl-4-chlorophenyl, 3-fluoro-4-trifluoromethylphenyl, 4-trifluoromethylphenyl, 3-methyl-4-trifluoromethylphenyl, or -3-chloro-4-fluorophenyl.

Thus in some embodiments, R¹ in Formula V or Formula VI is —CH₂-aryl, for example, —CH₂-difluorophenyl, —CH₂-3,4-difluorophenyl, —CH₂-4-chlorophenyl, —CH₂-3-chloro-4-fluorophenyl, —CH₂-4-chloro-3-fluorophenyl, —CH₂-dichlorophenyl, —CH₂-3,4-dichlorophenyl, —CH₂-3-methyl-4-chlorophenyl, —CH₂-3-fluoro-4-trifluoromethylphenyl, —CH₂-4-trifluoromethylphenyl, —CH₂-3-methyl-4-trifluoromethylphenyl, or —CH₂-3-chloro-4-fluorophenyl.

In other embodiments, R¹ in Formula V or Formula VI is CH(OH)-aryl, for example, —CH(OH)-4-chlorophenyl, —CH(OH)-3,4-dichlorophenyl, —CH(OH)-3,4-difluorophenyl, —CH(OH)-3-fluoro-4-chlorophenyl, —CH(OH)-3-chloro-4-fluorophenyl, —CH(OH)-3-methyl-4-chlorophenyl, —CH(OH)-3-fluoro-4-trifluoromethylphenyl, —CH(OH)-4-trifluoromethylphenyl, —CH(OH)-3-methyl-4-trifluoromethylphenyl, or —CH(OH)-3-chloro-4-fluorophenyl.

In other embodiments, R¹ in Formula V or Formula VI is —CH(halo)-aryl, for example, —CH(F)-4-chlorophenyl, —CH(F)-3,4-dichlorophenyl, —CH(F)-3,4-difluorophenyl, —CH(F)-3-fluoro-4-chlorophenyl, —CH(F)-3-chloro-4-fluorophenyl, —CH(F)-3-methyl-4-chlorophenyl, —CH(F)-3-fluoro-4-trifluoromethylphenyl, —CH(F)-4-trifluoromethylphenyl, —CH(F)-3-methyl-4-trifluoromethylphenyl, or —CH(F)-3-chloro-4-fluorophenyl.

In other embodiments, R¹ in Formula V or Formula VI is —CH(NH₂)-aryl, for example, —CH(NH₂)-4-chlorophenyl, —CH(NH₂)-3,4-dichlorophenyl, —CH(NH₂)-3,4-difluorophenyl, —CH(NH₂)-3-fluoro-4-chlorophenyl, —CH(NH₂)-3-chloro-4-fluorophenyl, —CH(NH₂)-3-methyl-4-chlorophenyl, —CH(NH₂)-3-fluoro-4-trifluoromethylphenyl, —CH(NH₂)-4-trifluoromethylphenyl, —CH(NH₂)-3-methyl-4-trifluoromethylphenyl, or —CH(NH₂)-3-chloro-4-fluorophenyl.

In other embodiments, R¹ in Formula V or Formula VI is —CH(Me)-aryl, for example, —CH(Me)-4-chlorophenyl, —CH(Me)-3,4-dichlorophenyl, —CH(Me)-3,4-difluorophenyl, —CH(Me)-3-fluoro-4-chlorophenyl, —CH(Me)-3-chloro-4-fluorophenyl, —CH(Me)-3-methyl-4-chlorophenyl, —CH(Me)-3-fluoro-4-trifluoromethylphenyl, —CH(Me)-4-trifluoromethylphenyl, —CH(Me)-3-methyl-4-trifluoromethylphenyl, or —CH(Me)-3-chloro-4-fluorophenyl.

In other embodiments, R¹ in Formula V or Formula VI is —C(Me)(OH)-aryl, for example, —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, —CH(Me)(OH)-3-methyl-4-chlorophenyl, —CH(Me)(OH)-3-fluoro-4-trifluoromethylphenyl, —CH(Me)(OH)-4-trifluoromethylphenyl, —CH(Me)(OH)-3-methyl-4-trifluoromethylphenyl, or —CH(Me)(OH)-3-chloro-4-fluorophenyl.

In other embodiments, R¹ in Formula V or Formula VI is —CH(CH₂OH)-aryl, for example, —CH(CH₂OH)-4-chlorophenyl, —CH(CH₂OH)-3,4-dichlorophenyl, —CH(CH₂OH)-3,4-difluorophenyl, —CH(CH₂OH)-3-fluoro-4-chlorophenyl, —CH(CH₂OH)-3-chloro-4-fluorophenyl, —CH(CH₂OH)-3-methyl-4-chlorophenyl, —CH(CH₂OH)-3-fluoro-4-trifluoromethylphenyl, —CH(CH₂OH)-4-trifluoromethylphenyl, —CH(CH₂OH)-3-methyl-4-trifluoromethylphenyl, or —CH(CH₂OH)-3-chloro-4-fluorophenyl.

In some aspects, R¹ in Formula V or Formula VI is —C₁-C₆alk-heteroaryl, for example, —C₁alk-heteroaryl, —C₂alk-heteroaryl, —C₃alk-heteroaryl, —C₄alk-heteroaryl, —C₅alk-heteroaryl, —C₆alk-heteroaryl, —CH₂heteroaryl, —CH(OH)-heteroaryl, —CH(F)-heteroaryl, —CH(NH₂)-heteroaryl, —CH(Me)-heteroaryl, —C(Me)(OH)-heteroaryl, —CH(CH₂OH)-heteroaryl and the like. In some embodiments the -heteroaryl is 5-chlorothiophen-2-yl, and R¹ is —CH₂-5-chlorothiophen-2-yl, —CH(OH)-5-chlorothiophen-2-yl, —CH(F)-5-chlorothiophen-2-yl, —CH(NH₂)-5-chlorothiophen-2-yl, —CH(Me)-5-chlorothiophen-2-yl, —C(Me)(OH)-5-chlorothiophen-2-yl, or —CH(CH₂OH)-5-chlorothiophen-2-yl.

In some aspects, R¹ in Formula V or Formula VI is —C₁-C₆alk-C≡CH, for example, —C₁alk-C≡CH, —C₂alk-C≡CH, —C₃alk-C≡CH, —C₄alk-C≡CH, —C₅alk-C≡CH, —C₆alk-C≡CH, ethynyl, propargyl, —CH(OH)—C≡CH, —CH(F)—C≡CH, —CH(NH₂)—C≡CH, —CH(Me)-C≡CH, —C(Me)(OH)—C≡CH, —CH(CH₂OH)—C≡CH and the like.

In some aspects, R¹ in Formula V or Formula VI is —C₁-C₆alk-C≡C—C₁-C₆alkyl, for example, —C₁alk-C≡C—C₁alkyl, —C₂alk-C≡C—C₁alkyl, —C₃alk-C≡C—C₁alkyl, —C₄alk-C≡C—C₁alkyl, —C₅alk-C≡C—C₁alkyl, —C₆alk-C≡C—C₁alkyl, —C₁alk-C≡C—C₂alkyl, —C₂alk-C≡C—C₂alkyl, —C₃alk-C≡C—C₂alkyl, —C₄alk-C≡C—C₂alkyl, —C₅alk-C≡C—C₂alkyl, —C₆alk-C≡C—C₂alkyl, —C₁alk-C≡C—C₃alkyl, —C₂alk-C≡C—C₃alkyl, —C₃alk-C≡C—C₃alkyl, —C₄alk-C≡C—C₃alkyl, —C₅alk-C≡C—C₃alkyl, —C₆alk-C≡C—C₃alkyl, —C₁alk-C≡C—C₄alkyl, —C₂alk-C≡C—C₄alkyl, —C₃alk-C≡C—C₄alkyl, —C₄alk-C≡C—C₄alkyl, —C₅alk-C≡C—C₄alkyl, —C₆alk-C≡C—C₄alkyl, —C₁alk-C≡C—C₅alkyl, —C₂alk-C≡C—C₅alkyl, —C₃alk-C≡C—C₅alkyl, —C₄alk-C≡C—C₅alkyl, —C₅alk-C≡C—C₅alkyl, —C₆alk-C≡C—C₅alkyl, —C₁alk-C≡C—C₆alkyl, —C₂alk-C≡C—C₆alkyl, —C₃alk-C≡C—C₆alkyl, —C₄alk-C≡C—C₆alkyl, —C₅alk-C≡C—C₆alkyl, —C₆alk-C≡C—C₆alkyl, propynyl, butynyl, —CH(OH)—C≡C—C₁-C₆alkyl, —CH(F)—C≡C—C₁-C₆alkyl, —CH(NH₂)—C≡C—C₁-C₆alkyl, —CH(Me)-C≡C—C₁-C₆alkyl, —C(Me)(OH)—C≡C—C₁-C₆alkyl, —CH(CH₂OH)—C≡C—C₁-C₆alkyl, and the like. In some embodiments wherein —C₁-C₆alk-C≡C—C₁-C₆alkyl is —C₁-C₆alk-C≡C—CH₃, R¹ is —CH(OH)—C≡C—CH₃, —CH(F)—C≡C—CH₃, —CH(NH₂)—C≡C—CH₃, —CH(Me)-C≡C—CH₃, or —C(Me)(OH)—C≡C—CH₃, —CH(CH₂OH)—C≡C—CH₃. In some embodiments, R¹ is —CH(OH)—C≡C—CH₃. In other embodiments, R¹ is —CH(F)—C≡C—CH₃. In yet other embodiments, R¹ is —CH(NH₂)—C≡C—CH₃. In some embodiments, R¹ is —CH(Me)-C≡C—CH₃. In other embodiments, R¹ is —CH(OH)(Me)-C≡C—CH₃. In yet other embodiments, R¹ is —CH(CH₂OH)(Me)-C≡C—CH₃.

In some aspects, R¹ in Formula V or Formula VI is —C₁-C₆alk-C≡C—C₁-C₆haloalkyl, for example, —C₁alk-C≡C—C₁haloalkyl, —C₂alk-C≡C—C₁haloalkyl, —C₃alk-C≡C—C₁haloalkyl, —C₄alk-C≡C—C₁haloalkyl, —C₅alk-C≡C—C₁haloalkyl, —C₆alk-C≡C—C₁haloalkyl, —C₁alk-C≡C—C₂haloalkyl, —C₂alk-C≡C—C₂haloalkyl, —C₃alk-C≡C—C₂haloalkyl, —C₄alk-C≡C—C₂haloalkyl, —C₅alk-C≡C—C₂haloalkyl, —C₆alk-C≡C—C₂haloalkyl, —C₁alk-C≡C—C₃haloalkyl, —C₂alk-C≡C—C₃haloalkyl, —C₃alk-C≡C—C₃haloalkyl, —C₄alk-C≡C—C₃haloalkyl, —C₅alk-C≡C—C₃haloalkyl, —C₆alk-C≡C—C₃haloalkyl, —C₁alk-C≡C—C₄haloalkyl, —C₂alk-C≡C—C₄haloalkyl, —C₃alk-C≡C—C₄haloalkyl, —C₄alk-C≡C—C₄haloalkyl, —C₅alk-C≡C—C₄haloalkyl, —C₆alk-C≡C—C₄haloalkyl, —C₁alk-C≡C—C₅haloalkyl, —C₂alk-C≡C—C₅haloalkyl, —C₃alk-C≡C—C₅haloalkyl, —C₄alk-C≡C—C₅haloalkyl, —C₅alk-C≡C—C₅haloalkyl, —C₆alk-C≡C—C₅haloalkyl, —C₁alk-C≡C—C₆haloalkyl, —C₂alk-C≡C—C₆haloalkyl, —C₃alk-C≡C—C₆haloalkyl, —C₄alk-C≡C—C₆haloalkyl, —C₅alk-C≡C—C₆haloalkyl, —C₆alk-C≡C—C₆haloalkyl, —CH(OH)—C≡C—C₁-C₆haloalkyl, —CH(F)—C≡C—C₁-C₆haloalkyl, —CH(NH₂)—C≡C—C₁-C₆haloalkyl, —CH(Me)-C≡C—C₁-C₆haloalkyl, —C(Me)(OH)—C≡C—C₁-C₆haloalkyl, —C(Me)(CH₂OH)—C≡C—C₁-C₆haloalkyl, and the like. In some embodiments wherein —C₁-C₆alk-C≡C—C₁-C₆haloalkyl is —C₁-C₆alk-C≡C—CF₃, R¹ is —CH(OH)—C≡C—CF₃, —CH(F)—C≡C—CF₃, —CH(NH₂)—C≡C—CF₃, —CH(Me)-C≡C—CF₃, —C(Me)(OH)—C≡C—CF₃, —C(Me)(CH₂OH)—C≡C—CF₃, and the like. Thus, in some embodiments, R¹ is —CH(OH)—C≡C—CF₃.

In some aspects, R¹ in Formula V or Formula VI is —C₁-C₆alk-C≡C—C₃-C₆cycloalkyl, for example, C₁alk-C≡C—C₃cycloalkyl, —C₁alk-C≡C—C₄cycloalkyl, —C₁alk-C≡C—C₅-cycloalkyl, —C₁alk-C≡C—C₆cycloalkyl, —C₂alk-C≡C—C₃cycloalkyl, —C₂alk-C≡C—C₄cycloalkyl, —C₂alk-C≡C—C₅cycloalkyl, —C₂alk-C≡C—C₆cycloalkyl, —C₃alk-C≡C—C₃cycloalkyl, —C₃alk-C≡C—C₄cycloalkyl, —C₃alk-C≡C—C₅cycloalkyl, —C₃alk-C≡C—C₆cycloalkyl, —C₄alk-C≡C—C₃cycloalkyl, —C₄alk-C≡C—C₄cycloalkyl, —C₄alk-C≡C—C₅cycloalkyl, —C₄alk-C≡C—C₆cycloalkyl, —C₅alk-C≡C—C₃cycloalkyl, —C₅alk-C≡C—C₄cycloalkyl, —C₅alk-C≡C—C₅cycloalkyl, —C₅alk-C≡C—C₆cycloalkyl, —C₆alk-C≡C—C₃cycloalkyl, —C₆alk-C≡C—C₄cycloalkyl, —C₆alk-C≡C—C₅cycloalkyl, —C₆alk-C≡C—C₆cycloalkyl, —CH(OH)—C≡C—C₃-C₆cycloalkyl, —CH(F)—C≡C—C₃-C₆cycloalkyl, —CH(NH₂)—C≡C—C₃-C₆cycloalkyl, —CH(Me)-C≡C—C₃-C₆cycloalkyl, —C(Me)(OH)—C≡C—C₃-C₆cycloalkyl, or —C(Me)(CH₂OH)—C≡C—C₃-C₆cycloalkyl. In some embodiments wherein —C₁-C₆alk-C≡C—C₃-C₆cycloalkyl is —C₁-C₆alk-C≡C-cyclopropyl, R¹ is —CH(OH)—C≡C-cyclopropyl, —CH(F)—C≡C-cyclopropyl, —CH(NH₂)—C≡C-cyclopropyl, —CH(Me)-C≡C-cyclopropyl, —C(Me)(OH)—C≡C-cyclopropyl, —C(Me)(CH₂OH)—C≡C— cyclopropyl, and the like. Thus, in some embodiments, R¹ is —CH(OH)—C≡C-cyclopropyl.

In compounds of the present disclosure that are compounds of Formula V, R² is H or halo.

Thus, in some embodiments, R² in Formula V is H.

In other embodiments, R² in Formula V is halo, for example F, Cl, Br, or I. In some embodiments, R² is F.

In compounds of the present disclosure of Formula V or Formula VI, R³ is H, halo, —C₁-C₆alkyl, or NH₂. Thus in some embodiments, R³ is H. In other embodiments, R³ is halo, for example F, Cl, Br, or I, with —Cl being preferred. In other embodiments, R³ is —C₁-C₆alkyl, for example, methyl, ethyl, propyl, isopropyl, butyl, isobutyl, s-butyl, t-butyl, pentyl, and the like. Thus, in some embodiments, R³ is methyl (Me). In yet other embodiments, R³ is NH₂.

In compounds of the present disclosure of Formula V or Formula VI, R⁴ is NH₂ or CH₃. In some embodiments, R⁴ is NH₂. In other embodiments, R⁴ is CH₃.

Preferred embodiments of the compounds of Formula V are those in which R¹ is —CH(OH)-aryl, —CH(Me)-aryl, —C(Me)(OH)-aryl, —CH(CH₂OH)-aryl, —C(Me)(OH)-heteroaryl, or —CH(OH)—C≡C—C₃-C₆cycloalkyl; R² is H or F; R³ is H, and R⁴ is NH₂.

Particularly preferred embodiments of the compounds of Formula V are those wherein R¹ is —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, —CH(Me)(OH)-3-methyl-4-chlorophenyl, —CH(Me)(OH)-3-fluoro-4-trifluoromethylphenyl, —CH(Me)(OH)-4-trifluoromethylphenyl, —CH(Me)(OH)-3-methyl-4-trifluoromethylphenyl, —CH(Me)(OH)-3-chloro-4-fluorophenyl, —CH(Me)-4-chlorophenyl, —CH(CH₂OH)-4-chlorophenyl, —C(Me)(OH)-5-chlorothiophen-2-yl, or —CH(OH)—C≡C-cyclopropyl; R² is H or F; R³ is H; and R⁴ is NH₂.

Preferred embodiments of the compounds of Formula VI are those in which R¹ is —CH(OH)-aryl, —CH(Me)-aryl, —C(Me)(OH)-aryl, —CH(CH₂OH)-aryl, —C(Me)(OH)-heteroaryl, or —CH(OH)—C≡C—C₃-C₆cycloalkyl; R³ is H; and R⁴ is NH₂.

Particularly preferred embodiments of the compounds of Formula VI are those wherein R¹ is —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, —CH(Me)(OH)-3-methyl-4-chlorophenyl, —CH(Me)(OH)-3-fluoro-4-trifluoromethylphenyl, —CH(Me)(OH)-4-trifluoromethylphenyl, —CH(Me)(OH)-3-methyl-4-trifluoromethylphenyl, —CH(Me)(OH)-3-chloro-4-fluorophenyl, —CH(Me)-4-chlorophenyl, —CH(CH₂OH)-4-chlorophenyl, —C(Me)(OH)-5-chlorothiophen-2-yl, or —CH(OH)—C≡C-cyclopropyl; R³ is H; and R⁴ is NH₂.

In some aspects, the present disclosure is directed to compounds of formula VA-1

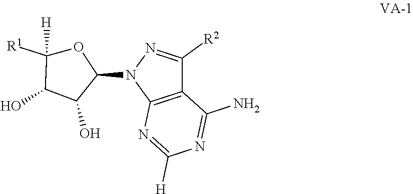

wherein R¹ is —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, —CH(Me)(OH)-3-methyl-4-chlorophenyl, —CH(Me)(OH)-3-fluoro-4-trifluoromethylphenyl, —CH(Me)(OH)-4-trifluoromethylphenyl, —CH(Me)(OH)-3-methyl-4-trifluoromethylphenyl, —CH(Me)(OH)-3-chloro-4-fluorophenyl, —CH(Me)-4-chlorophenyl, —CH(CH₂OH)-4-chlorophenyl, —C(Me)(OH)-5-chlorothiophen-2-yl, or —CH(OH)—C≡C-cyclopropyl; and R² is H or F.

In some aspects, the present disclosure is directed to compounds of formula VA-2

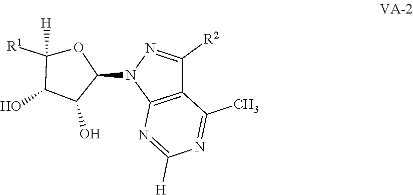

wherein R¹ is —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, —CH(Me)(OH)-3-methyl-4-chlorophenyl, —CH(Me)(OH)-3-fluoro-4-trifluoromethylphenyl, —CH(Me)(OH)-4-trifluoromethylphenyl, —CH(Me)(OH)-3-methyl-4-trifluoromethylphenyl, —CH(Me)(OH)-3-chloro-4-fluorophenyl, —CH(Me)-4-chlorophenyl, —CH(CH₂OH)-4-chlorophenyl, —C(Me)(OH)-5-chlorothiophen-2-yl, or —CH(OH)—C≡C-cyclopropyl; and R² is H or F.

In other aspects, the present disclosure is dureceted to compounds of formula VB-1

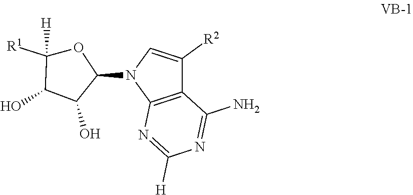

wherein R¹ is —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, —CH(Me)(OH)-3-methyl-4-chlorophenyl, —CH(Me)(OH)-3-fluoro-4-trifluoromethylphenyl, —CH(Me)(OH)-4-trifluoromethylphenyl, —CH(Me)(OH)-3-methyl-4-trifluoromethylphenyl, —CH(Me)(OH)-3-chloro-4-fluorophenyl, —CH(Me)-4-chlorophenyl, —CH(CH₂OH)-4-chlorophenyl, —C(Me)(OH)-5-chlorothiophen-2-yl, or —CH(OH)—C≡C-cyclopropyl; and R² is H or F.

In some embodiments, the compounds of formula VB-1 are those wherein R¹ is —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, —CH(Me)(OH)-3-methyl-4-chlorophenyl, —CH(Me)(OH)-3-fluoro-4-trifluoromethylphenyl, —CH(Me)(OH)-4-trifluoromethylphenyl, —CH(Me)(OH)-3-methyl-4-trifluoromethylphenyl, —CH(Me)(OH)-3-chloro-4-fluorophenyl, —CH(Me)-4-chlorophenyl, —CH(CH₂OH)-4-chlorophenyl, —C(Me)(OH)-5-chlorothiophen-2-yl, or —CH(OH)—C≡C-cyclopropyl; and R² is F.

In some embodiments, the compounds of formula VB-1 are those wherein R¹ is —C(Me)(OH)-3-chloro-4-fluorophenyl, —CH(Me)(OH)-3-methyl-4-chlorophenyl, —CH(Me)(OH)-3-fluoro-4-trifluoromethylphenyl, —CH(Me)(OH)-3-methyl-4-trifluoromethylphenyl, —CH(Me)(OH)-3-chloro-4-fluorophenyl, —CH(Me)-4-chlorophenyl, —CH(CH₂OH)-4-chlorophenyl, —C(Me)(OH)-5-chlorothiophen-2-yl, or —CH(OH)—C≡C-cyclopropyl; and R² is H or F.

In other aspects, the present disclosure is dureceted to compounds of formula VB-2

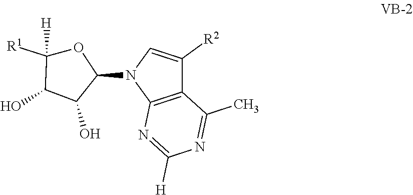

wherein R¹ is —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, —CH(Me)(OH)-3-methyl-4-chlorophenyl, —CH(Me)(OH)-3-fluoro-4-trifluoromethylphenyl, —CH(Me)(OH)-4-trifluoromethylphenyl, —CH(Me)(OH)-3-methyl-4-trifluoromethylphenyl, —CH(Me)(OH)-3-chloro-4-fluorophenyl, —CH(Me)-4-chlorophenyl, —CH(CH₂OH)-4-chlorophenyl, —C(Me)(OH)-5-chlorothiophen-2-yl, or —CH(OH)—C≡C-cyclopropyl; and R² is H or F.

In some embodiments, the compounds of formula VB-2 are those wherein R¹ is —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, —CH(Me)(OH)-3-methyl-4-chlorophenyl, —CH(Me)(OH)-3-fluoro-4-trifluoromethylphenyl, —CH(Me)(OH)-4-trifluoromethylphenyl, —CH(Me)(OH)-3-methyl-4-trifluoromethylphenyl, —CH(Me)(OH)-3-chloro-4-fluorophenyl, —CH(Me)-4-chlorophenyl, —CH(CH₂OH)-4-chlorophenyl, —C(Me)(OH)-5-chlorothiophen-2-yl, or —CH(OH)—C≡C-cyclopropyl; and R² is F.

In other embodiments, the compounds of formula VB-2 are those wherein R¹ is —C(Me)(OH)-3,4-difluorophenyl, —C(Me)(OH)-3-fluoro-4-chlorophenyl, —C(Me)(OH)-3-chloro-4-fluorophenyl, —CH(Me)(OH)-3-methyl-4-chlorophenyl, —CH(Me)(OH)-3-fluoro-4-trifluoromethylphenyl, —CH(Me)(OH)-4-trifluoromethylphenyl, —CH(Me)(OH)-3-methyl-4-trifluoromethylphenyl, —CH(Me)(OH)-3-chloro-4-fluorophenyl, —CH(Me)-4-chlorophenyl, —CH(CH₂OH)-4-chlorophenyl, —C(Me)(OH)-5-chlorothiophen-2-yl, or —CH(OH)—C≡C-cyclopropyl; and R² is H.

In yet other aspects, the present disclosure is directed to compounds of formula VI-A-1

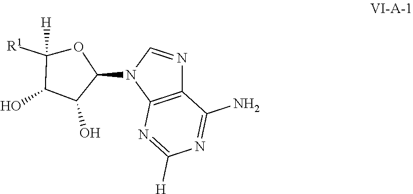

wherein R¹ is —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, —CH(Me)(OH)-3-methyl-4-chlorophenyl, —CH(Me)(OH)-3-fluoro-4-trifluoromethylphenyl, —CH(Me)(OH)-4-trifluoromethylphenyl, —CH(Me)(OH)-3-methyl-4-trifluoromethylphenyl, —CH(Me)(OH)-3-chloro-4-fluorophenyl, —CH(Me)-4-chlorophenyl, —CH(CH₂OH)-4-chlorophenyl, —C(Me)(OH)-5-chlorothiophen-2-yl, or —CH(OH)—C≡C-cyclopropyl.

In yet other aspects, the present disclosure is dureceted to compounds of formula VI-A-2

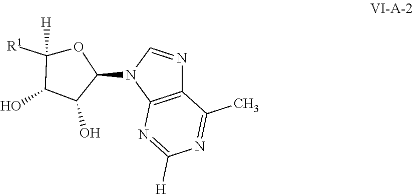

wherein R¹ is —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, —CH(Me)(OH)-3-methyl-4-chlorophenyl, —CH(Me)(OH)-3-fluoro-4-trifluoromethylphenyl, —CH(Me)(OH)-4-trifluoromethylphenyl, —CH(Me)(OH)-3-methyl-4-trifluoromethylphenyl, —CH(Me)(OH)-3-chloro-4-fluorophenyl, —CH(Me)-4-chlorophenyl, —CH(CH₂OH)-4-chlorophenyl, —C(Me)(OH)-5-chlorothiophen-2-yl, or —CH(OH)—C≡C-cyclopropyl.

Stereoisomers of compounds of Formula V and Formula VI are also contemplated.

Pharmaceutically acceptable salts and solvates of the compounds of Formula V and Formula VI are also within the scope of the disclosure.

The oximes of the present disclosure, i.e., the compounds of Formula I or II, wherein Q=O, and R¹, R², R³, R⁴, R⁵ have the values described above,

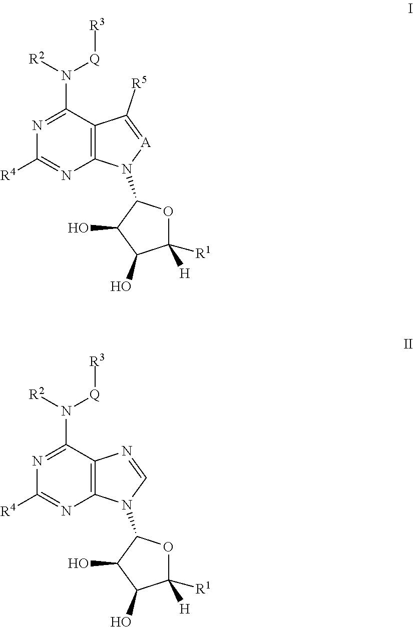

can be converted under physiological conditions, or by methods known to those skilled in the art, into the corresponding amino compounds, shown below.

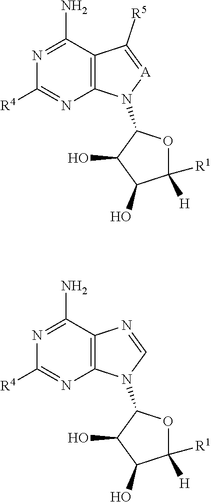

Thus, the amino compounds corresponding to oximes of Formula I or II, as well as the amino compounds corresponding to the oxime-containing subgenera of Formula I or II, described herein, are also encompassed by the disclosure. Subgenera of these amino compounds, corresponding to oxime containing subgenera of IA. IA-3, IA-4, IB, IB-3, IB-4, IB-5, IB-6, IE, and IF, IIA, IIB, IIE, IIF, IIG, IIG-3, and IIG-4 described herein, are also encompassed by the present disclosure. The individual amino compounds corresponding to the oximes set forth in Table A below are also encompassed by the disclosure.

### Pharmaceutical Compositions and Methods of Administration

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

A pharmaceutical composition of the invention typically contains an active ingredient (i.e., a compound of the disclosure) of the present invention or a pharmaceutically acceptable salt and/or coordination complex thereof, and one or more pharmaceutically acceptable excipients, carriers, including but not limited to inert solid diluents and fillers, diluents, sterile aqueous solution and various organic solvents, permeation enhancers, solubilizers and adjuvants.

Described below are non-limiting exemplary pharmaceutical compositions and methods for preparing the same.

### Pharmaceutical Compositions for Oral Administration.

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof, carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof, polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-lOoleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, F-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, F-caprolactone and isomers thereof, δ-valerolactone and isomers thereof, β-butyrolactone and isomers thereof, and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25% o, 50%), 100% o, or up to about 200%>by weight, based on the combined weight of the drug, and other excipients. If desired, very small amounts of solubilizer may also be used, such as 5%>, 2%>, 1%) or even less. Typically, the solubilizer may be present in an amount of about 1%>to about 100%, more typically about 5%>to about 25%>by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical Compositions for Topical (e.g. Transdermal) Delivery.

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins, 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent or through inhalation. Compounds can also be administered intraadiposally or intrathecally.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, a compound of the invention is administered in a single dose.

Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; U.S. Pat. Nos. 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

### Methods of Use

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

As used herein, the term “IC₅₀” refers to the half maximal inhibitory concentration of an inhibitor in inhibiting biological or biochemical function. This quantitative measure indicates how much of a particular inhibitor is needed to inhibit a given biological process (or component of a process, i.e. an enzyme, cell, cell receptor or microorganism) by half. In other words, it is the half maximal (50%) inhibitory concentration (IC) of a substance (50% IC, or IC50). EC50 refers to the plasma concentration required for obtaining 50%>of a maximum effect in vivo.

In some embodiments, the subject methods utilize a PRMT5 inhibitor with an IC50 value of about or less than a predetermined value, as ascertained in an in vitro assay. In some embodiments, the PRMT5 inhibitor inhibits PRMT5 a with an IC50 value of about 1 nM or less, 2 nM or less, 5 nM or less, 7 nM or less, 10 nM or less, 20 nM or less, 30 nM or less, 40 nM or less, 50 nM or less, 60 nM or less, 70 nM or less, 80 nM or less, 90 nM or less, 100 nM or less, 120 nM or less, 140 nM or less, 150 nM or less, 160 nM or less, 170 nM or less, 180 nM or less, 190 nM or less, 200 nM or less, 225 nM or less, 250 nM or less, 275 nM or less, 300 nM or less, 325 nM or less, 350 nM or less, 375 nM or less, 400 nM or less, 425 nM or less, 450 nM or less, 475 nM or less, 500 nM or less, 550 nM or less, 600 nM or less, 650 nM or less, 700 nM or less, 750 nM or less, 800 nM or less, 850 nM or less, 900 nM or less, 950 nM or less, 1 μM or less, 1.1 μM or less, 1.2 μM or less, 1.3 μM or less, 1.4 μM or less, 1.5 μM or less, 1.6 μM or less, 1.7 μM or less, 1.8 μM or less, 1.9 μM or less, 2 μM or less, 5 μM or less, 10 μM or less, 15 μM or less, 20 μM or less, 25 μM or less, 30 μM or less, M or less, 50 μM, 60 μM, 70 μM, 80 μM, 90 μM, 100 μM, 200 μM, 300 μM, 400 μM, or 500 μM, or less, (or a number in the range defined by and including any two numbers above).

In some embodiments, the PRMT5 inhibitor selectively inhibits PRMT5 a with an IC50 value that is at least 2, 3, 4, 5, 6, 7, 8, 9, 10, 15, 20, 25, 30, 35, 40, 45, 50, 100, or 1000 times less (or a number in the range defined by and including any two numbers above) than its IC50 value against one, two, or three other PRMTs.

In some embodiments, the PRMT5 inhibitor selectively inhibits PRMT5 a with an IC50 value that is less than about 1 nM, 2 nM, 5 nM, 7 nM, 10 nM, 20 nM, 30 nM, 40 nM, 50 nM, 60 nM, 70 nM, 80 nM, 90 nM, 100 nM, 120 nM, 140 nM, 150 nM, 160 nM, 170 nM, 180 nM, 190 nM, 200 nM, 225 nM, 250 nM, 275 nM, 300 nM, 325 nM, 350 nM, 375 nM, 400 nM, 425 nM, 450 nM, 475 nM, 500 nM, 550 nM, 600 nM, 650 nM, 700 nM, 750 nM, 800 nM, 850 nM, 900 nM, 950 nM, 1 μM, 1.1 μM, 1.2 μM, 1.3 μM, 1.4 μM, 1.5 μM, 1.6 μM, 1.7 μM, 1.8 μM, 1.9 μM, 2 μM, 5 μM, 10 μM, 15 μM, 20 μM, 25 μM, 30 μM, 40 μM, 50 μM, 60 μM, 70 μM, 80 μM, 90 μM, 100 μM, 200 μM, 300 μM, 400 μM, or 500 μM (or in the range defined by and including any two numbers above), and said IC50 value is at least 2, 3, 4, 5, 6, 7, 8, 9, 10, 15, 20, 25, 30, 35, 40, 45, 50, 100, or 1000 times less (or a number in the range defined by and including any two numbers above) than its IC50 value against one, two or three other PRMTs.

The subject methods are useful for treating a disease or disorder associated with PRMT5. Any disease or disorder that results directly or indirectly from an abnormal activity or expression level of PRMT5 can be an intended disease condition.

Examples of diseases or disorders associated with PRMT5 that may be treated with compounds of the disclosure include lupus erythematosus, ankylosing spondylitis, hidradenitis suppurativa, C3 glomerulopathy, ANCA associated vasculitis, focal segmental glomerulosclerosis, chronic inflammatory demyelinating polyneuropathy, amyotrophic lateral sclerosis, relapsing polychondroitis, polymyositis, bullous dermatoses, bronchiectasis, pulmonary hypertension, cystic fibrosis, pulmonary fibrosis, transplanted organs or tissue; graft-versus-host diseases brought about by transplantation; acute respiratory distress syndrome; adult respiratory distress syndrome; influenza; COVID-19 (coronavirus disease); systemic erythematosus; Hashimoto's thyroiditis; lymphocytic thyroiditis; multiple sclerosis; myasthenia gravis, uveitis; posterior uveitis; uveitis associated with Behcet's disease; uveomeningitis syndrome; allergic encephalomyelitis; chronic allograftvasculopathy; rheumatic fever and post-infectious glomerulonephritis, atopic dermatitis; osteomyelitis; contact dermatitis; eczematous dermatitis; seborrhoeic dermatitis; lichen planus; pemphigus; bullous pemphigoid; epidermolysis bullosa; urticaria; angioedema; vasculitis; erythema; cutaneous eosinophilia; acne; alopecia areata; keratoconjunctivitis; vernal conjunctivitis; keratitis; herpetic keratitis; dystrophia epithelialis corneae; corneal leukoma; ocular pemphigus; Mooren's ulcer; ulcerative keratitis; scleritis; Graves' ophthalmopathy; Vogt-Koyanagi-Harada syndrome; sarcoidosis; pollen allergies; reversible obstructive airway disease; bronchial asthma; allergic asthma; intrinsic asthma; extrinsic asthma; dust asthma; chronic or inveterate asthma; late asthma and airway hyper-responsiveness; bronchitis; gastric ulcers; ischemic bowel diseases, necrotizing enterocolitis; intestinal lesions associated with thermal burns; celiac diseases; proctitis; eosinophilic gastroenteritis, Crohn's disease; ulcerative colitis; vascular damage caused by ischemic diseases and thrombosis; atherosclerosis; fatty heart; myocarditis; cardiac infarction; arteriosclerosis, aortitis syndrome; cachexia due to viral disease; vascular thrombosis; migraine; rhinitis; interstitial nephritis; IgA-induced nephropathy; Goodpasture's syndrome; hemolytic-uremic syndrome; diabetic nephropathy; glomerulosclerosis; glomerulonephritis; multiple myositis; Guillain-Barre syndrome; Meniere's disease; polyneuritis; multiple neuritis; mononeuritis; radiculopathy; hyperthyroidism; Basedow's disease; thyrotoxicosis; pure red cell aplasia; aplastic anemia; hypoplastic anemia; idiopathic thrombocytopenic purpura; autoimmune hemolytic anemia; agranulocytosis; pernicious anemia; megaloblastic anemia; anerythroplasia; osteoporosis; sarcoidosis; fibroid lung; idiopathic interstitial pneumonia; dermatomyositis; leukoderma vulgaris; ichthyosis vulgaris; photoallergic sensitivity; polyarteritis nodosa; Huntington's chorea; Sydenham's chorea; myocardosis, Wegener's granuloma; Sjogren's syndrome; adiposis; eosinophilic fascitis; lesions of gingiva, periodontium, alveolar bone, substantia ossea dentis; male pattern alopecia or alopecia senihs; muscular dystrophy; pyoderma; Sezary's syndrome; chronic adrenal insufficiency; Addison's disease; ischemia-reperfusion injury of organs which occurs upon preservation; endotoxin shock; pseudomembranous colitis; colitis caused by drug or radiation; ischemic acute renal insufficiency; chronic renal insufficiency; inflammatory lung injury, pulmonary emphysema; cataracta; siderosis; retinitis pigmentosa; vitreal scarring; inflammatory eye disease; corneal alkali burn; dermatitis erythema; ballous dermatitis; cement dermatitis; gingivitis; periodontitis; sepsis; pancreatitis; hypobaropathy; autoimmune hepatitis; primary biliary cirrhosis; sclerosing cholangitis; partial liver resection; acute liver necrosis; cirrhosis; alcoholic cirrhosis; hepatic failure; fulminant hepatic failure; late-onset hepatic failure; “acute-on-chronic” liver failure.

In some embodiments, the disease or disorder is inflammatory and hyperproliferative skin diseases, or cutaneous manifestations of immunologically-mediated disorders.

In yet further embodiments, the disease or disorder is rejection of transplanted organs or tissue; graft-versus-host diseases brought about by transplantation; autoimmune syndromes, multiple sclerosis, myasthenia gravis; pollen allergies; type I diabetes; prevention of psoriasis; Crohn's disease; ulcerative colitis, acute respiratory distress syndrome; adult respiratory distress syndrome; influenza; or post-infectious autoimmune diseases including rheumatic fever and post-infectious glomerulonephritis.

In yet further embodiments the disease or disorder is one of influenza, COVID-19 (coronavirus disease); ulcerative colitis, multiple sclerosis, transplant rejection, acute respiratory distress syndrome or adult respiratory distress syndrome.

In yet further embodiments the disease or disorder is type I or type II diabetes.

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

Compounds of the disclosure can be prepared, for example, by reference to the following schemes.

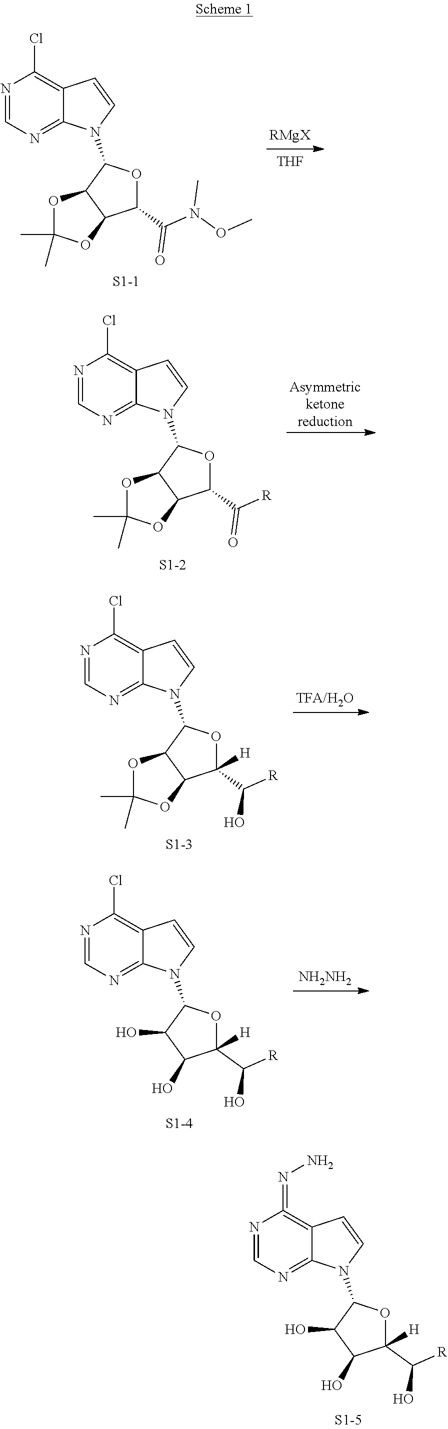

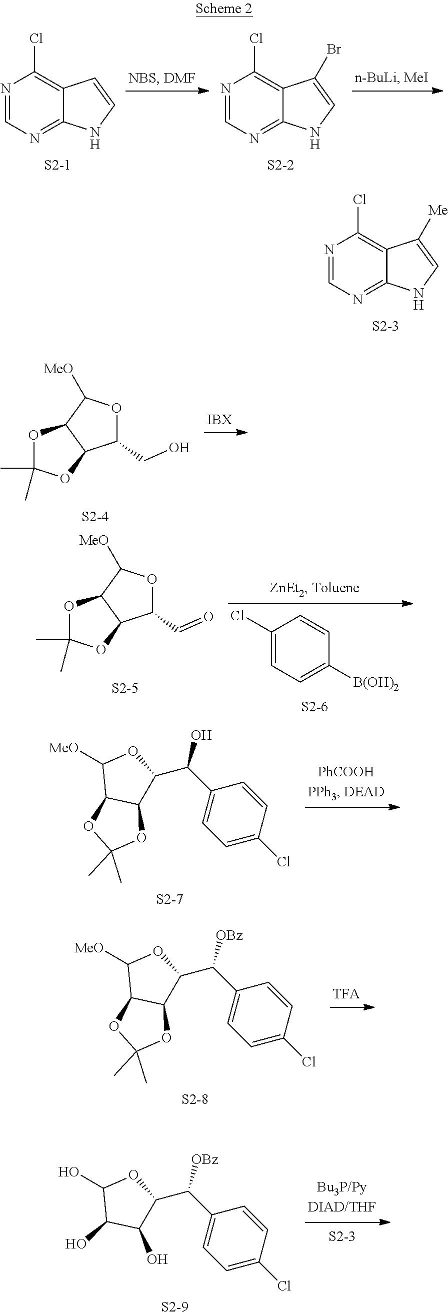

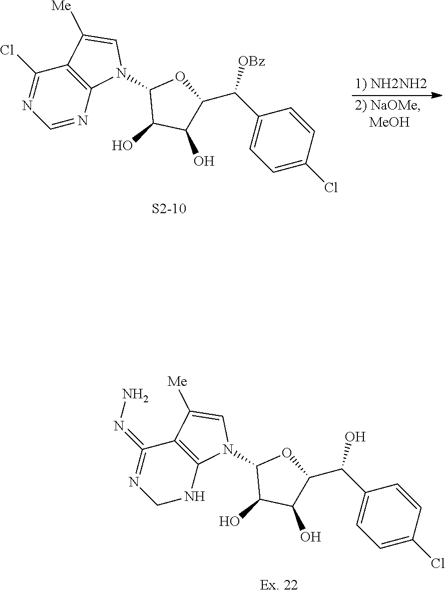

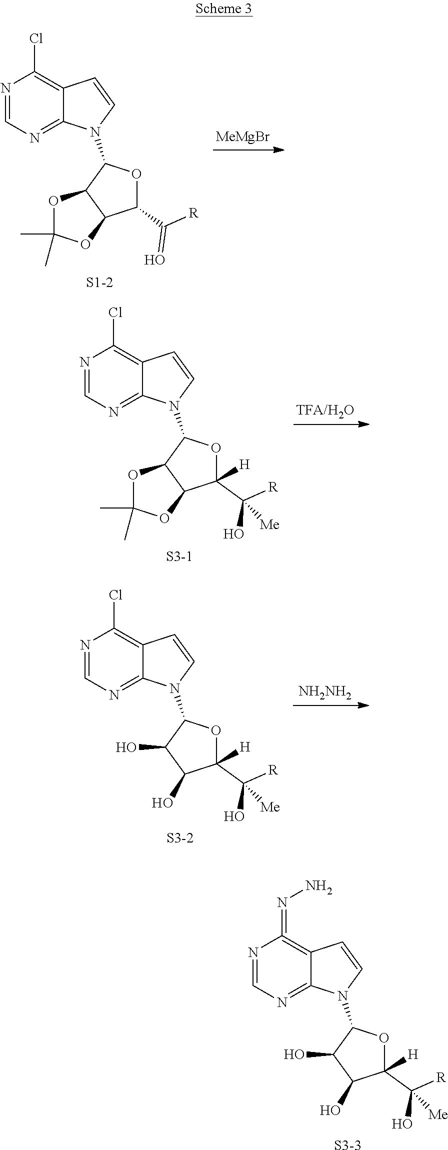

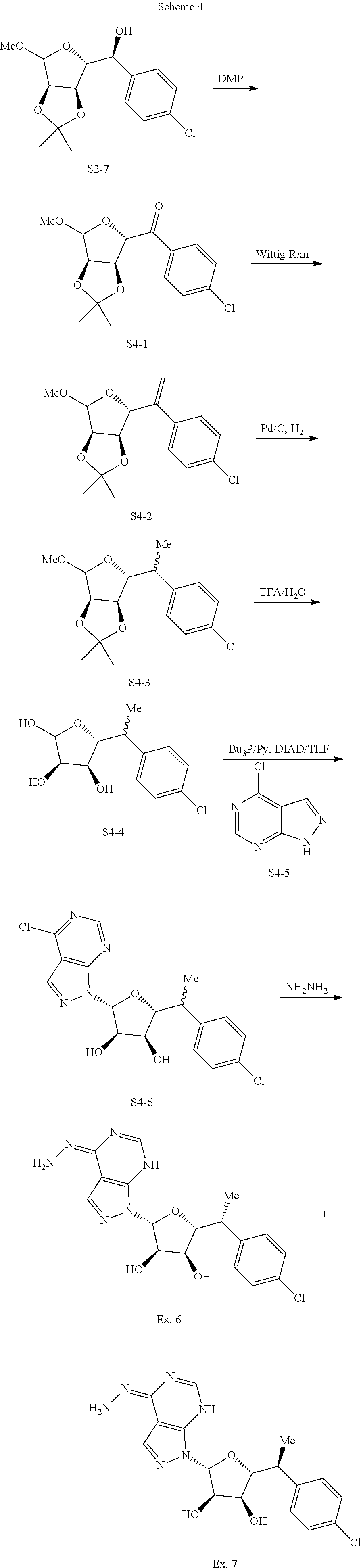

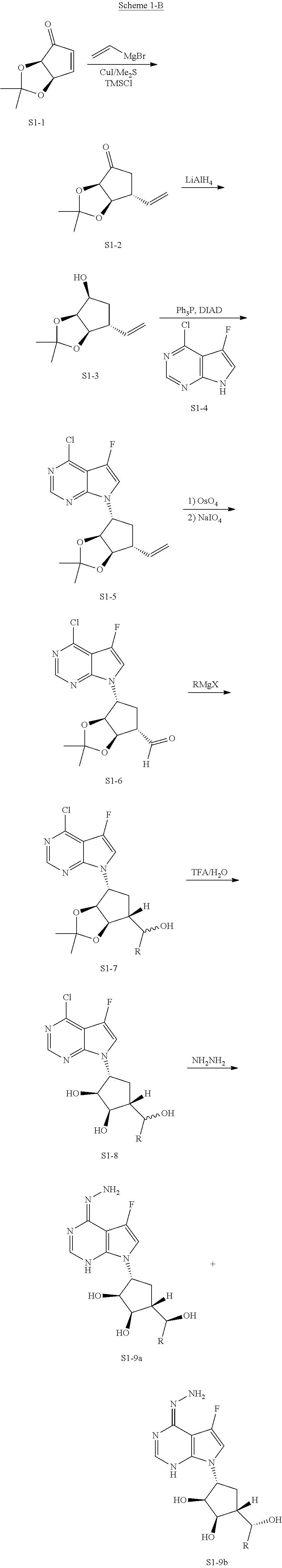

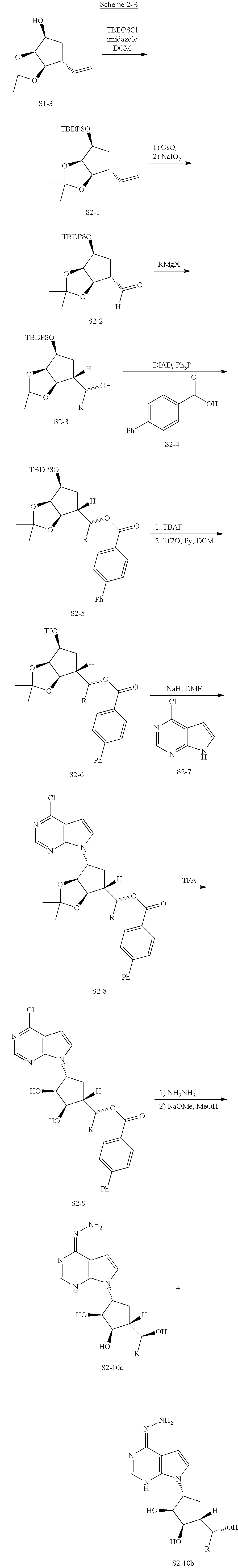

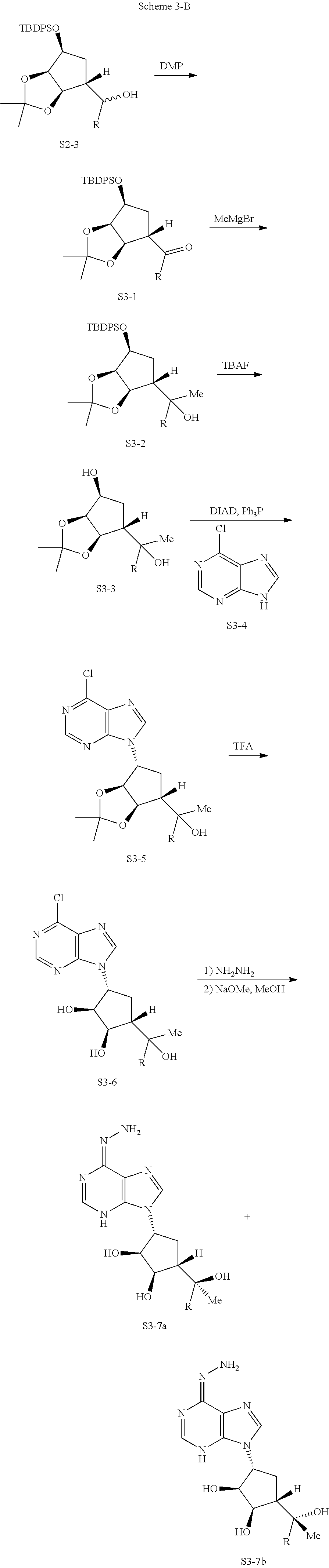

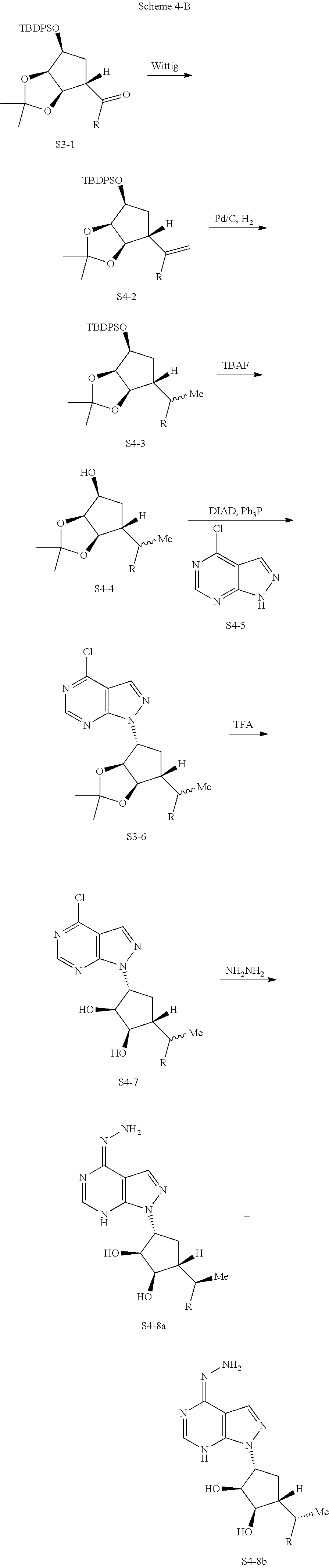

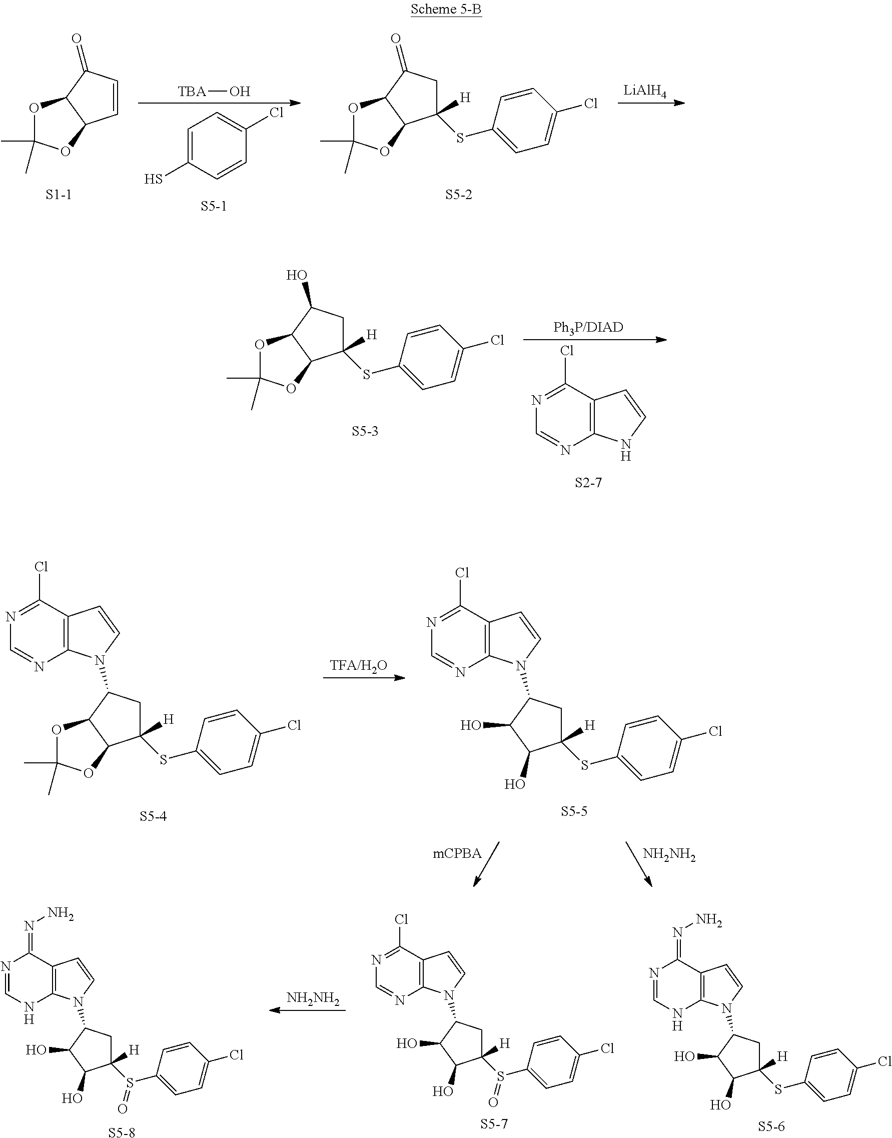

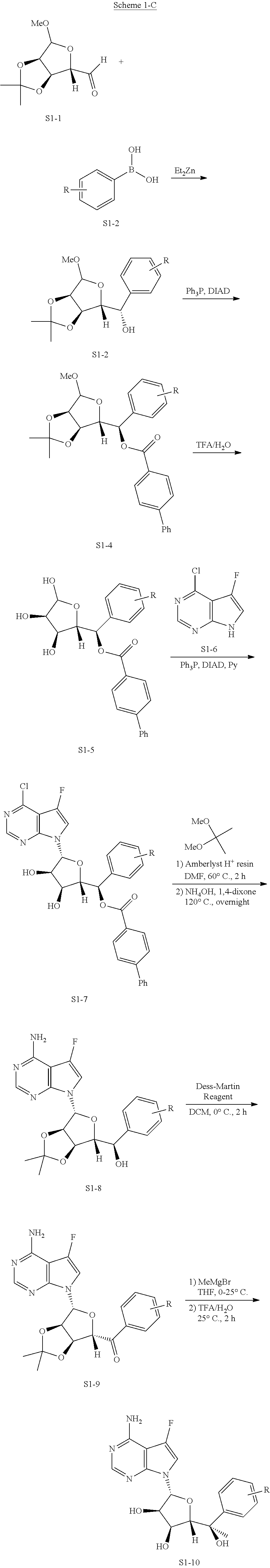

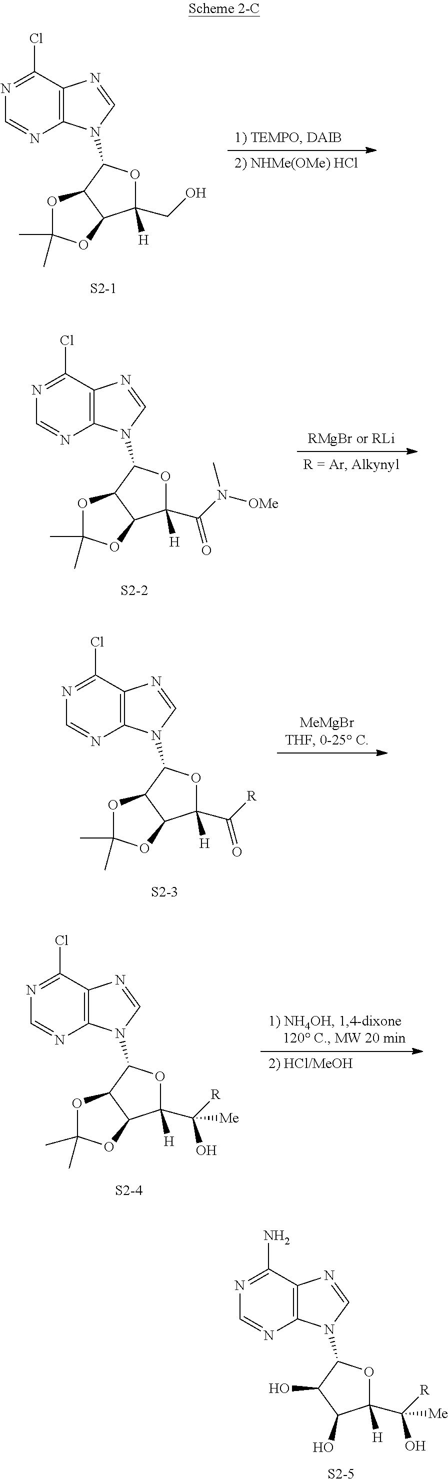

Compounds of the disclosure of Formula I and Formula II include, for example, the compounds identified in Table A.

TABLE A

| Ex. # | Structures | MW | Chemical Name |
|---|---|---|---|
| 1 | 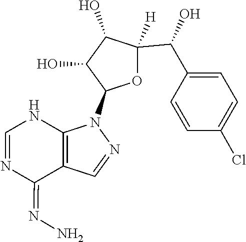 [SMILES: N/N=c1/nc[nH]c2c1cnn2[C@@H]1O[C@H]([C@H](O)c2ccc(Cl)cc2)[C@@H](O)[C@H]1O] | 392.8 | (2R,3S,4R,5R)-2-((R)- (4-chlorophenyl) (hydroxy)methyl)-5-(4- hydrazineylidene-4,7-dihydro-1H- pyrazolo[3,4-d]pyrimidin-1- yl)tetrahydrofuran-3,4-diol |
| 2 | 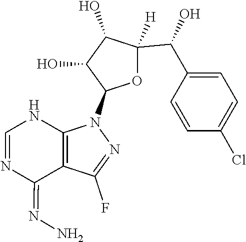 [SMILES: N/N=c1/nc[nH]c2c1c(F)nn2[C@@H]1O[C@H]([C@H](O)c2ccc(Cl)cc2)[C@@H](O)[C@H]1O] | 410.8 | (2R,3S,4R,5R)-2-((R)-(4- chlorophenyl)(hydroxy)methyl)- 5-(3-fluoro-4-hydrazineylidene- 4,7-dihydro-1H- pyrazolo[3,4-d]pyrimidin-1- yl)tetrahydrofuran-3,4-diol |
| 3 | 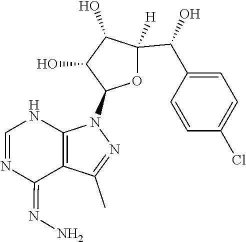 [SMILES: Cc1nn([C@@H]2O[C@H]([C@H](O)c3ccc(Cl)cc3)[C@@H](O)[C@H]2O)c2[nH]cn/c(=N/N)c12] | 406.8 | (2R,3S,4R,5R)-2-((R)-(4- chlorophenyl)(hydroxy)methyl)-5-(4- hydrazineylidene-3-methyl-4,7- dihydro-1H-pyrazolo[3,4-d]pyrimidin- 1-yl)tetrahydrofuran-3,4-diol |
| 4 | 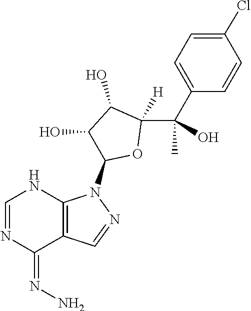 [SMILES: C[C@@](O)(c1ccc(Cl)cc1)[C@H]1O[C@@H](n2ncc3/c(=N\N)nc[nH]c32)[C@H](O)[C@@H]1O] | 406.8 | (2S,3S,4R,5R)-2-((R)-1- (4-chlorophenyl)-1-hydroxyethyl)- 5-(4-hydrazineylidene-4,7- dihydro-1H-pyrazolo[3,4-d]pyrimidin- 1-yl)tetrahydrofuran-3,4-diol |
| 5 | 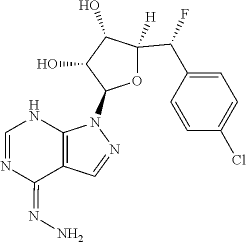 [SMILES: N/N=c1/nc[nH]c2c1cnn2[C@@H]1O[C@H]([C@H](F)c2ccc(Cl)cc2)[C@@H](O)[C@H]1O] | 394.8 | (2S,3S,4R,5R)-2-((R)-(4- chlorophenyl)fluoromethyl)-5- (4-hydrazineylidene-4,7-dihydro- 1H-pyrazolo[3,4-d]pyrimidin-1- yl)tetrahydrofuran-3,4-diol |
| 6 | 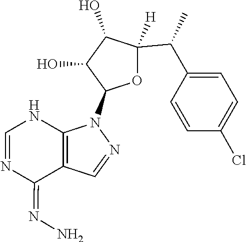 [SMILES: C[C@H](c1ccc(Cl)cc1)[C@H]1O[C@@H](n2ncc3/c(=N\N)nc[nH]c32)[C@H](O)[C@@H]1O] | 390.8 | (2R,3S,4R,5R)-2-((R)-1-(4- chlorophenyl)ethyl)-5- (4-hydrazineylidene- 4,7-dihydro-1H-pyrazolo[3,4-d] pyrimidin-1- yl)tetrahydrofuran-3,4-diol |
| 7 | 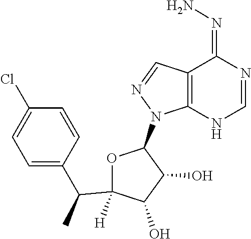 [SMILES: C[C@@H](c1ccc(Cl)cc1)[C@H]1O[C@@H](n2ncc3/c(=N\N)nc[nH]c32)[C@H](O)[C@@H]1O] | 390.8 | (2R,3S,4R,5R)-2-((S)-1-(4- chlorophenyl)ethyl)-5- (4-hydrazineylidene-4,7-dihydro- 1H-pyrazolo[3,4-d]pyrimidin-1- yl)tetrahydrofuran-3,4-diol |
| 8 | 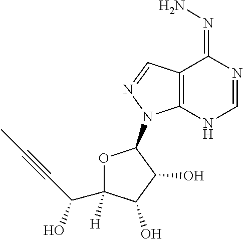 [SMILES: CC#C[C@@H](O)[C@H]1O[C@@H](n2ncc3/c(=N\N)nc[nH]c32)[C@H](O)[C@@H]1O] | 320.3 | (2R,3R,4S,5R)-2- (4-hydrazineylidene-4,7- dihydro-1H-pyrazolo[3,4-d]pyrimidin- 1-yl)-5-((R)-1-hydroxybut-2- yn-1-yl)tetrahydrofuran-3,4-diol |
| 9 | 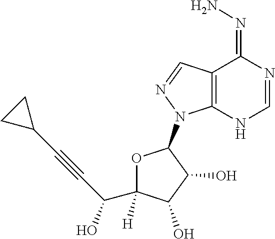 [SMILES: N/N=c1/nc[nH]c2c1cnn2[C@@H]1O[C@H]([C@H](O)C#CC2CC2)[C@@H](O)[C@H]1O] | 346.3 | (2R,3S,4R,5R)-2-((R)-3- cyclopropy1-1- hydroxyprop-2-yn-1-yl)-5-(4- hydrazineylidene-4,7-dihydro-1H- pyrazolo[3,4-d]pyrimidin-1- yl)tetrahydrofuran-3,4-diol |
| 10 | 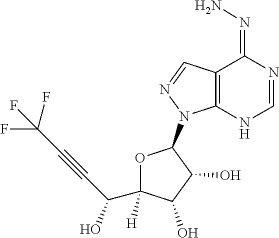 [SMILES: N/N=c1/nc[nH]c2c1cnn2[C@@H]1O[C@H]([C@H](O)C#CC(F)(F)F)[C@@H](O)[C@H]1O] | 374.3 | (2R,3R,4S,5R)-2- (4-hydrazineylidene-4,7-dihydro- 1H-pyrazolo[3,4-d]pyrimidin-1-yl)- 5-((R)-4,4,4-trifluoro-1-hydroxybut- 2-yn-1-yl)tetrahydrofuran-3,4-diol |
| 11 | 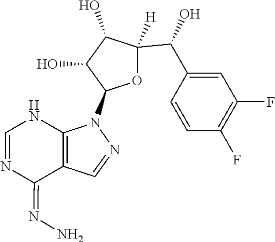 [SMILES: N/N=c1/nc[nH]c2c1cnn2[C@@H]1O[C@H]([C@H](O)c2ccc(F)c(F)c2)[C@@H](O)[C@H]1O] | 394.3 | (2R,3S,4R,5R)-2-((R)-(3,4- difluorophenyl)(hydroxy)methyl)- 5-(4-hydrazineylidene-4,7- dihydro-1H- pyrazolo[3,4-d]pyrimidin-1- yl)tetrahydrofuran-3,4-diol |
| 12 | 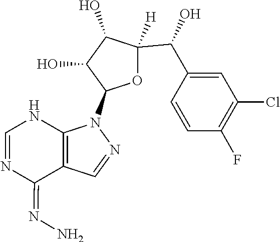 [SMILES: N/N=c1/nc[nH]c2c1cnn2[C@@H]1O[C@H]([C@H](O)c2ccc(F)c(Cl)c2)[C@@H](O)[C@H]1O] | 410.8 | (2R,3S,4R,5R)-2-((R)-(3-chloro-4- fluorophenyl)(hydroxy)methyl)-5-(4- hydrazineylidene-4,7-dihydro-1H- pyrazolo[3,4-d]pyrimidin-1- yl)tetrahydrofuran-3,4-diol |
| 13 | 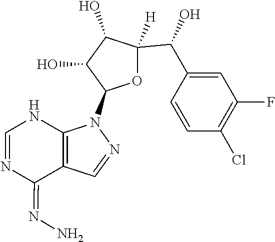 [SMILES: N/N=c1/nc[nH]c2c1cnn2[C@@H]1O[C@H]([C@H](O)c2ccc(Cl)c(F)c2)[C@@H](O)[C@H]1O] | 410.8 | (2R,3S,4R,5R)-2-((R)-(4-chloro-3- fluorophenyl)(hydroxy)methyl)-5-(4- hydrazineylidene-4,7-dihydro-1H- pyrazolo[3,4-d]pyrimidin-1- yl)tetrahydrofuran-3,4-diol |
| 14 | 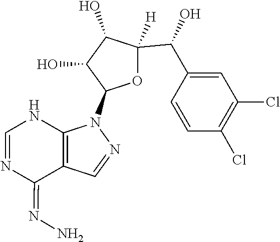 [SMILES: N/N=c1/nc[nH]c2c1cnn2[C@@H]1O[C@H]([C@H](O)c2ccc(Cl)c(Cl)c2)[C@@H](O)[C@H]1O] | 427.2 | (2R,3S,4R,5R)-2-((R)-(3,4- dichlorophenyl)(hydroxy)methyl)- 5-(4-hydrazineylidene-4,7-dihydro- 1H-pyrazolo[3,4-d]pyrimidin-1- yl)tetrahydrofuran-3,4-diol |
| 15 | 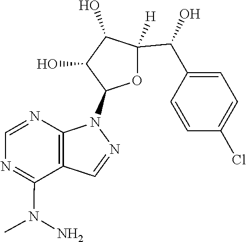 [SMILES: CN(N)c1ncnc2c1cnn2[C@@H]1O[C@H]([C@H](O)c2ccc(Cl)cc2)[C@@H](O)[C@H]1O] | 406.8 | (2R,3S,4R,5R)-2-((R)-(4- chlorophenyl)(hydroxy)methyl)- 5-(4-(1-methylhydrazineyl)-1H- pyrazolo[3,4-d] pyrimidin-1-yl)ltetrahydrofuran-3,4-diol |
| 16 | 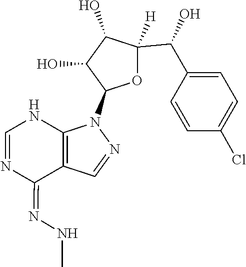 [SMILES: CN/N=c1/nc[nH]c2c1cnn2[C@@H]1O[C@H]([C@H](O)c2ccc(Cl)cc2)[C@@H](O)[C@H]1O] | 406.8 | (2R,3S,4R,5R)-2-((R)-(4- chlorophenyl)(hydroxy)methyl)- 5-(4-(2-methylhydrazineylidene)- 4,7-dihydro-1H-pyrazolo[3,4-d] pyrimidin-1-yl)tetrahydrofuran-3,4-diol |
| 17 | 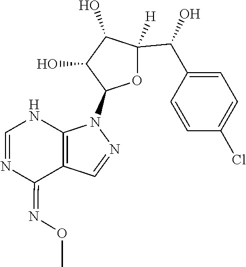 [SMILES: CO/N=c1/nc[nH]c2c1cnn2[C@@H]1O[C@H]([C@H](O)c2ccc(Cl)cc2)[C@@H](O)[C@H]1O] | 407.8 | 1-((2R,3R,4S,5R)-5-((R)-(4- chlorophenyl)(hydroxy)methyl)- 3,4-dihydroxytetrahydrofuran-2-yl)- 1,7-dihydro-4H- pyrazolo[3,4-d]pyrimidin- 4-one O-methyl oxime |
| 18 | 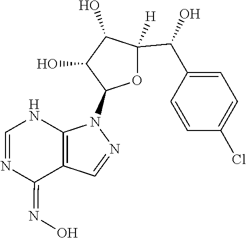 [SMILES: O/N=c1/nc[nH]c2c1cnn2[C@@H]1O[C@H]([C@H](O)c2ccc(Cl)cc2)[C@@H](O)[C@H]1O] | 393.8 | 1-((2R,3R,4S,5R)-5-((R)-(4- chlorophenyl)(hydroxy)methyl)-3,4- dihydroxytetrahydrofuran-2-yl)-1,7- dihydro- 4H-pyrazolo[3,4-d]pyrimidin- 4-one oxime |
| 19 | 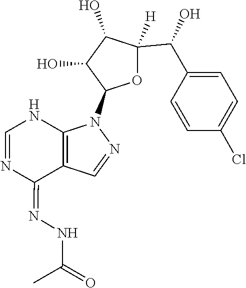 [SMILES: CC(=O)N/N=c1/nc[nH]c2c1cnn2[C@@H]1O[C@H]([C@H](O)c2ccc(Cl)cc2)[C@@H](O)[C@H]1O] | 434.8 | N′-1-((2R,3R,4S,5R)-5-((R)-(4- chlorophenyl)(hydroxy)methyl)-3,4- dihydroxytetrahydrofuran-2-yl)-1,7- dihydro-4H-pyrazolo[3,4-d]pyrimidin- 4-ylidene)acetohydrazide |
| 20 | 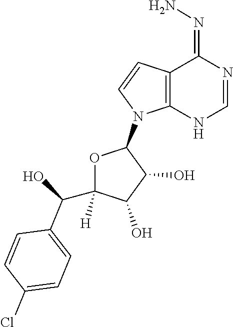 [SMILES: N/N=c1/nc[nH]c2c1ccn2[C@@H]1O[C@H]([C@H](O)c2ccc(Cl)cc2)[C@@H](O)[C@H]1O] | 391.8 | (2R,3S,4R,5R)-2-((R)-(4- chlorophenyl)(hydroxy)methyl)- 5-(4-hydrazineylidene-1,4-dihydro- 7H-pyrrolo[2,3-d]pyrimidin-7- yl)tetrahydrofuran-3,4-diol |
| 21 | 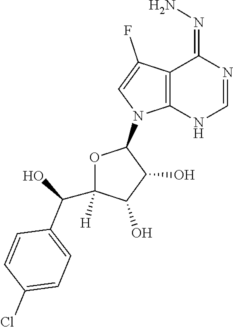 [SMILES: N/N=c1/nc[nH]c2c1c(F)cn2[C@@H]1O[C@H]([C@H](O)c2ccc(Cl)cc2)[C@@H](O)[C@H]1O] | 409.8 | (2R,3S,4R,5R)-2-((R)-(4- chlorophenyl)(hydroxy)methyl)- 5-(5-fluoro-4-hydrazineylidene- 1,4-dihydro-7H-pyrrolo[2,3-d] pyrimidin-7-yl)tetrahydrofuran-3,4-diol |
| 22 | 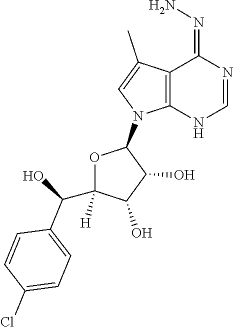 [SMILES: Cc1cn([C@@H]2O[C@H]([C@H](O)c3ccc(Cl)cc3)[C@@H](O)[C@H]2O)c2[nH]cn/c(=N/N)c12] | 405.8 | (2R,3S,4R,5R)-2-((R)-(4- chlorophenyl)(hydroxy)methyl)- 5-(4-hydrazineylidene-5- methyl-1,4-dihydro-7H- pyrrolo[2,3-d]pyrimidin-7- yl)tetrahydrofuran-3,4-diol |
| 23 | 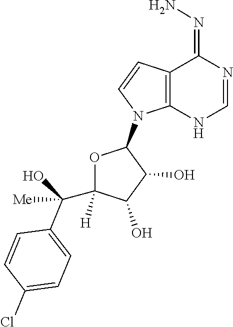 [SMILES: C[C@@](O)(c1ccc(Cl)cc1)[C@H]1O[C@@H](n2ccc3/c(=N\N)nc[nH]c32)[C@H](O)[C@@H]1O] | 405.8 | (2S,3S,4R,5R)-2-((R)-1- (4-chlorophenyl)- 1-hydroxyethyl)-5- (4-hydrazineylidene- 1,4-dihydro-7H- pyrrolo[2,3-d]pyrimidin- 7-yl)tetrahydrofuran-3,4-diol |
| 24 | 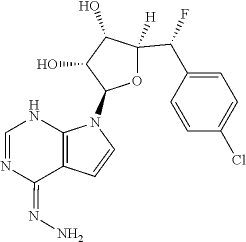 [SMILES: N/N=c1/nc[nH]c2c1ccn2[C@@H]1O[C@H]([C@H](F)c2ccc(Cl)cc2)[C@@H](O)[C@H]1O] | 393.8 | (2S,3S,4R,5R)-2-((R)-(4- chlorophenyl)fluoromethyl)- 5-(4-hydrazineylidene-1,4- dihydro-7H-pyrrolo[2,3-d]pyrimidin- 7-yl)tetrahydrofuran-3,4-diol |
| 25 | 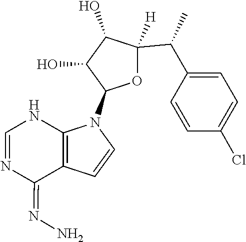 [SMILES: C[C@H](c1ccc(Cl)cc1)[C@H]1O[C@@H](n2ccc3/c(=N\N)nc[nH]c32)[C@H](O)[C@@H]1O] | 389.8 | (2R,3S,4R,5R)-2-((R)-1-(4- chlorophenyl)ethyl)-5- (4-hydrazineylidene-1,4-dihydro- 7H-pyrrolo[2,3-d]pyrimidin-7- yl)tetrahydrofuran-3,4-diol |
| 26 | 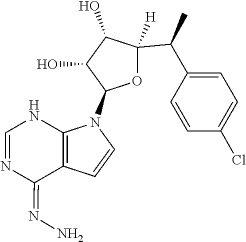 [SMILES: C[C@@H](c1ccc(Cl)cc1)[C@H]1O[C@@H](n2ccc3/c(=N\N)nc[nH]c32)[C@H](O)[C@@H]1O] | 389.8 | (2R,3S,4R,5R)-2-((S)-1-(4- chlorophenyl)ethyl)-5- (4-hydrazineylidene-1,4-dihydro- 7H-pyrrolo[2,3-d]pyrimidin-7- yl)tetrahydrofuran-3,4-diol |
| 27 | 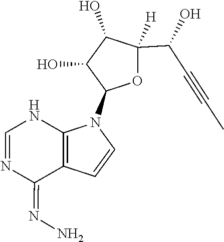 [SMILES: CC#C[C@@H](O)[C@H]1O[C@@H](n2ccc3/c(=N\N)nc[nH]c32)[C@H](O)[C@@H]1O] | 319.3 | (2R,3R,4S,5R)-2-(4-hydrazineylidene- 1,4-dihydro-7H- pyrrolo[2,3-d]pyrimidin-7-yl)-5-((R)- 1-hydroxybut-2-yn-1-yl)tetrahydrofuran- 3,4-diol |
| 28 | 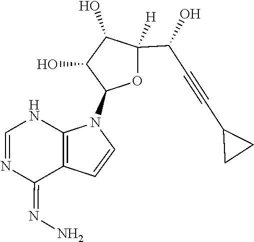 [SMILES: N/N=c1/nc[nH]c2c1ccn2[C@@H]1O[C@H]([C@H](O)C#CC2CC2)[C@@H](O)[C@H]1O] | 345.4 | (2R,3S,4R,5R)-2-((R)-3-cyclopropyl- 1-hydroxyprop-2-yn-1-yl)-5-(4- hydrazineylidene-1,4-dihydro-7H- pyrrolo[2,3-d]pyrimidin-7- yl)tetrahydrofuran-3,4-diol |
| 29 | 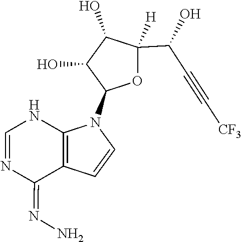 [SMILES: N/N=c1/nc[nH]c2c1ccn2[C@@H]1O[C@H]([C@H](O)C#CC(F)(F)F)[C@@H](O)[C@H]1O] | 373.3 | (2R,3R,4S,5R)-2- (4-hydrazineylidene-1,4- dihydro-7H- pyrrolo[2,3-d]pyrimidin-7-yl)-5- ((R)-4,4,4-trifluoro- 1-hydroxybut-2-yn-1- yl)tetrahydrofuran-3,4-diol |
| 30 | 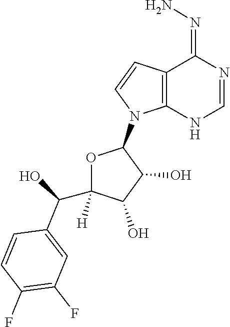 [SMILES: N/N=c1/nc[nH]c2c1ccn2[C@@H]1O[C@H]([C@H](O)c2ccc(F)c(F)c2)[C@@H](O)[C@H]1O] | 393.4 | (2R,3S,4R,5R)-2-((R)-(3,4- difluorophenyl)(hydroxy)methyl)-5-(4- hydrazineylidene-1,4-dihydro-7H- pyrrolo[2,3-d]pyrimidin-7- yl)tetrahydrofuran-3,4-diol |
| 31 | 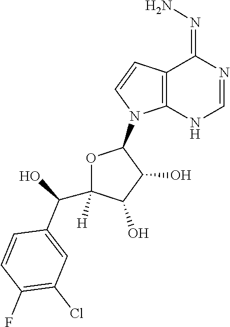 [SMILES: N/N=c1/nc[nH]c2c1ccn2[C@@H]1O[C@H]([C@H](O)c2ccc(F)c(Cl)c2)[C@@H](O)[C@H]1O] | 409.8 | (2R,3S,4R,5R)-2-((R)-(3-chloro-4- fluorophenyl)(hydroxy)methyl)-5-(4- hydrazineylidene-1,4-dihydro-7H- pyrrolo[2,3-d]pyrimidin-7- yl)tetrahydrofuran-3,4-diol |
| 32 | 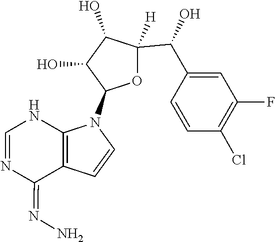 [SMILES: N/N=c1/nc[nH]c2c1ccn2[C@@H]1O[C@H]([C@H](O)c2ccc(Cl)c(F)c2)[C@@H](O)[C@H]1O] | 409.8 | (2R,3S,4R,5R)-2-((R)-(4-chloro-3- fluorophenyl)(hydroxy)methyl)-5-(4- hydrazineylidene-1,4-dihydro-7H- pyrrolo[2,3-d]pyrimidin-7- yl)tetrahydrofuran-3,4-diol |
| 33 | 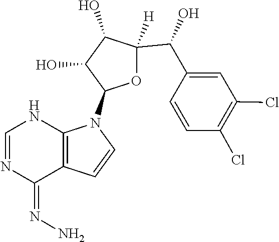 [SMILES: N/N=c1/nc[nH]c2c1ccn2[C@@H]1O[C@H]([C@H](O)c2ccc(Cl)c(Cl)c2)[C@@H](O)[C@H]1O] | 426.3 | (2R,3S,4R,5R)-2-((R)-(3,4- dichlorophenyl)(hydroxy)methyl)- 5-(4-hydrazineylidene-1,4-dihydro- 7H-pyrrolo[2,3-d]pyrimidin-7- yl)tetrahydrofuran-3,4-diol |
| 34 | 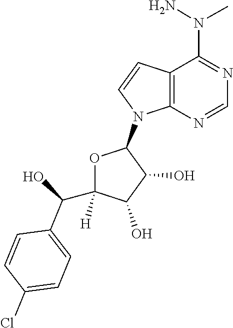 [SMILES: CN(N)c1ncnc2c1ccn2[C@@H]1O[C@H]([C@H](O)c2ccc(Cl)cc2)[C@@H](O)[C@H]1O] | 405.8 | (2R,3S,4R,5R)-2-((R)-(4- chlorophenyl)(hydroxy)methyl)- 5-(4-(1-methylhydrazineyl)- 7H-pyrrolo[2,3d]pyrimidin- 7-yl)tetrahydrofuran-3,4-diol |
| 35 | 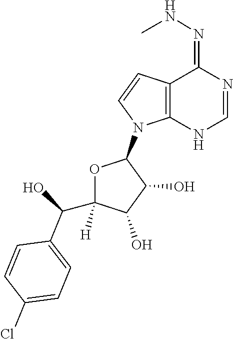 [SMILES: CN/N=c1/nc[nH]c2c1ccn2[C@@H]1O[C@H]([C@H](O)c2ccc(Cl)cc2)[C@@H](O)[C@H]1O] | 405.8 | (2R,3S,4R,5R)-2-((R)-(4- chlorophenyl)(hydroxy)methyl)- 5-(4-(2-methylhydrazineylidene)- 1,4-dihydro-7H- pyrrolo[2,3-d]pyrimidin-7- yl)tetrahydrofuran-3,4-diol |
| 36 | 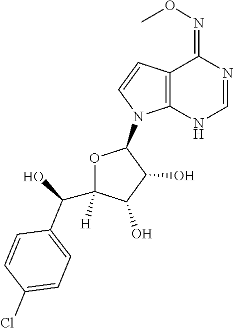 [SMILES: CO/N=c1/nc[nH]c2c1ccn2[C@@H]1O[C@H]([C@H](O)c2ccc(Cl)cc2)[C@@H](O)[C@H]1O] | 406.8 | 7-((2R,3R,4S,5R)-5-((R)-(4- chlorophenyl)(hydroxy)methyl)-3,4- dihydroxytetrahydrofuran-2-yl)- 1,7-dihydro-4H- pyrrolo[2,3-d]pyrimidin- 4-one O-methyl |
| 37 | 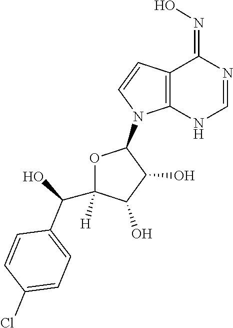 [SMILES: O/N=c1/nc[nH]c2c1ccn2[C@@H]1O[C@H]([C@H](O)c2ccc(Cl)cc2)[C@@H](O)[C@H]1O] | 392.8 | 7-((2R,3R,4S,5R)-5-((R)-(4- chlorophenyl)(hydroxy)methyl)-3,4- dihydroxytetrahydrofuran-2-yl)- 1,7-dihydro-4H- pyrrolo[2,3-d]pyrimidin-4-one oxime |
| 38 | 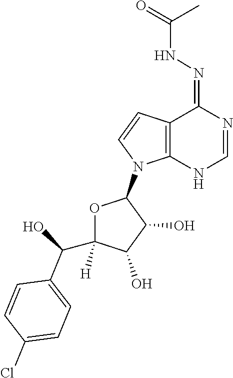 [SMILES: CC(=O)N/N=c1/nc[nH]c2c1ccn2[C@@H]1O[C@H]([C@H](O)c2ccc(Cl)cc2)[C@@H](O)[C@H]1O] | 433.8 | N′-(7-((2R,3R,4S,5R)-5-((R)-(4- chlorophenyl)(hydroxy)methyl)- 3,4-dihydroxytetrahydrofuran- 2-yl)-1,7-dihydro- 4H-pyrrolo[2,3-d]pyrimidin-4- ylidene)acetohydrazide |
| 39 | 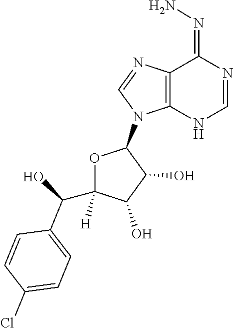 [SMILES: N/N=c1/nc[nH]c2c1ncn2[C@@H]1O[C@H]([C@H](O)c2ccc(Cl)cc2)[C@@H](O)[C@H]1O] | 392.8 | (2R,3S,4R,5R)-2-((R)-(4- chlorophenyl)(hydroxy)methyl)-5-(6- hydrazineylidene-3,6-dihydro-9H- purin-9-yl)tetrahydrofuran-3,4-diol |
| 40 | 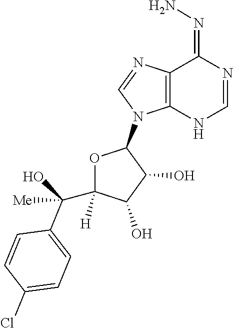 [SMILES: C[C@@](O)(c1ccc(Cl)cc1)[C@H]1O[C@@H](n2cnc3/c(=N\N)nc[nH]c32)[C@H](O)[C@@H]1O] | 406.8 | (2S,3S,4R,5R)-2-((R)-1- (4-chlorophenyl)- 1-hydroxyethyl)-5- (6-hydrazineylidene- 3,6-dihydro-9H-purin-9-yl) tetrahydrofuran-3,4-diol |
| 41 | 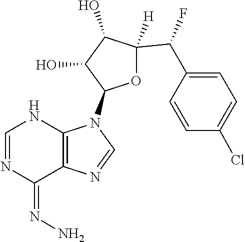 [SMILES: N/N=c1/nc[nH]c2c1ncn2[C@@H]1O[C@H]([C@H](F)c2ccc(Cl)cc2)[C@@H](O)[C@H]1O] | 394.8 | (2S,3S,4R,5R)-2-((R)-(4- chlorophenyl)fluoromethyl)- 5-(6-hydrazineylidene-3,6- dihydro-9H-purin-9- yl)tetrahydrofuran-3,4-diol |
| 42 | 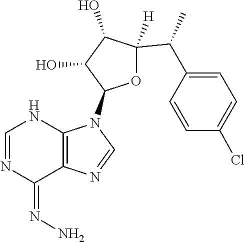 [SMILES: C[C@H](c1ccc(Cl)cc1)[C@H]1O[C@@H](n2cnc3/c(=N\N)nc[nH]c32)[C@H](O)[C@@H]1O] | 390.8 | (2R,3S,4R,5R)-2-((R)-1-(4- chlorophenyl)ethyl)-5- (6-hydrazineylidene-3,6- dihydro-9H-purin-9- yl)tetrahydrofuran-3,4-diol |
| 43 | 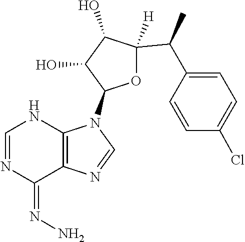 [SMILES: C[C@@H](c1ccc(Cl)cc1)[C@H]1O[C@@H](n2cnc3/c(=N\N)nc[nH]c32)[C@H](O)[C@@H]1O] | 390.8 | (2R,3S,4R,5R)-2-((S)-1-(4- chlorophenyl)ethyl)-5-(6- hydrazineylidene-3,6-dihydro- 9H-purin-9-yl)tetrahydrofuran- 3,4-diol |
| 44 | 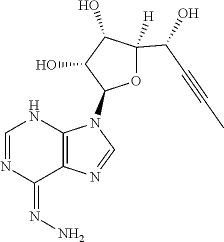 [SMILES: CC#C[C@@H](O)[C@H]1O[C@@H](n2cnc3/c(=N\N)nc[nH]c32)[C@H](O)[C@@H]1O] | 320.3 | (2R,3R,4S,5R)-2-(6- hydrazineylidene-3,6- dihydro-9H-purin-9-yl)-5- ((R)-1-hydroxybut- 2-yn-1-yl)tetrahydrofuran- 3,4-diol |
| 45 | 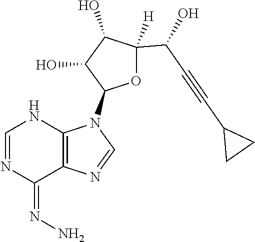 [SMILES: N/N=c1/nc[nH]c2c1ncn2[C@@H]1O[C@H]([C@H](O)C#CC2CC2)[C@@H](O)[C@H]1O] | 346.3 | (2R,3S,4R,5R)-2-((R)-3-cyclopropyl- 1-hydroxyprop-2-yn-1-yl)-5-(6- hydrazineylidene-3,6-dihydro- 9H-purin-9- yl)tetrahydrofuran-3,4-diol |
| 46 | 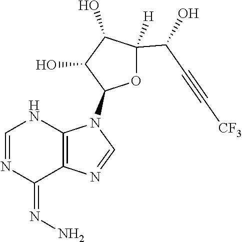 [SMILES: N/N=c1/nc[nH]c2c1ncn2[C@@H]1O[C@H]([C@H](O)C#CC(F)(F)F)[C@@H](O)[C@H]1O] | 374.3 | (2R,3R,4S,5R)-2-(-6- hydrazineylidene-3,6- dihydro-9H-purin-9-yl)-5-((R)- 4,4,4-trifluoro-1-hydroxybut- 2-yn-1-yl)tetrahydrofuran-3,4-diol |
| 47 | 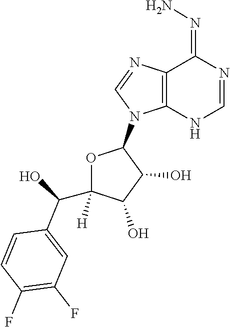 [SMILES: N/N=c1/nc[nH]c2c1ncn2[C@@H]1O[C@H]([C@H](O)c2ccc(F)c(F)c2)[C@@H](O)[C@H]1O] | 394.3 | (2R,3S,4R,5R)-2-((R)-(3,4- difluorophenyl)(hydroxy)methyl)- 5-(6-hydrazineylidene-3,6-dihydro- 9H-purin-9-yl)tetrahydrofuran- 3,4-diol |
| 48 | 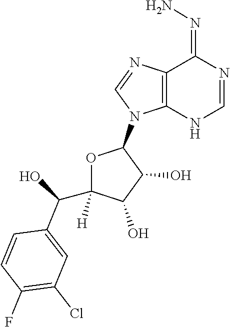 [SMILES: N/N=c1/nc[nH]c2c1ncn2[C@@H]1O[C@H]([C@H](O)c2ccc(F)c(Cl)c2)[C@@H](O)[C@H]1O] | 410.8 | (2R,3S,4R,5R)-2-((R)-(3-chloro-4- fluorophenyl)(hydroxy)methyl)-5-(6- hydrazineylidene-3,6-dihydro-9H- purin-9- yl)tetrahydrofuran-3,4-diol |
| 49 | 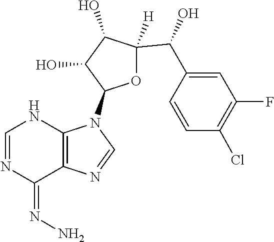 [SMILES: N/N=c1/nc[nH]c2c1ncn2[C@@H]1O[C@H]([C@H](O)c2ccc(Cl)c(F)c2)[C@@H](O)[C@H]1O] | 410.8 | (2R,3S,4R,5R)-2-((R)-(4-chloro-3- fluorophenyl)(hydroxy)methyl)- 5-(6-hydrazineylidene- 3,6-dihydro-9H-purin-9- yl)tetrahydrofuran-3,4-diol |
| 50 | 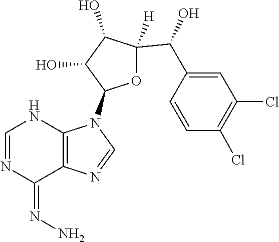 [SMILES: N/N=c1/nc[nH]c2c1ncn2[C@@H]1O[C@H]([C@H](O)c2ccc(Cl)c(Cl)c2)[C@@H](O)[C@H]1O] | 427.2 | (2R,3S,4R,5R)-2-((R)-(3,4- dichlorophenyl)(hydroxy)methyl)- 5-(6-hydrazineylidene- 3,6-dihydro-9H-purin-9- yl)tetrahydrofuran-3,4-diol |
| 51 | 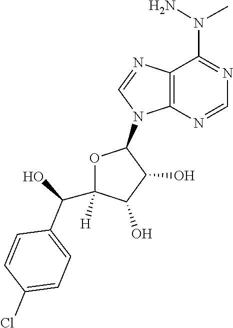 [SMILES: CN(N)c1ncnc2c1ncn2[C@@H]1O[C@H]([C@H](O)c2ccc(Cl)cc2)[C@@H](O)[C@H]1O] | 406.8 | (2R,3S,4R,5R)-2-((R)-(4- chlorophenyl)(hydroxy)methyl)- 5-(6-(1-methylhydrazineyl)- 9H-purin-9- yl)tetrahydrofuran-3,4-diol |
| 52 | 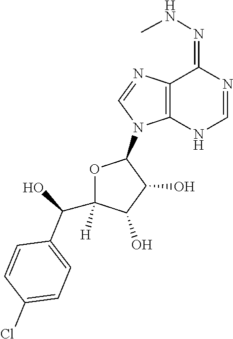 [SMILES: CN/N=c1/nc[nH]c2c1ncn2[C@@H]1O[C@H]([C@H](O)c2ccc(Cl)cc2)[C@@H](O)[C@H]1O] | 406.8 | (2R,3S,4R,5R)-2-((R)-(4- chlorophenyl)(hydroxy)methyl)- 5-(6-(2-methylhydrazineylidene)- 3,6-dihydro-9H- purin-9-yl)tetrahydrofuran-3,4-diol |
| 53 | 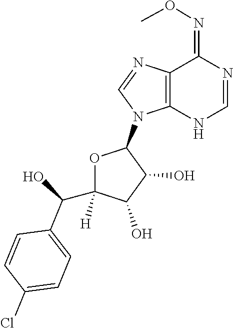 [SMILES: CO/N=c1/nc[nH]c2c1ncn2[C@@H]1O[C@H]([C@H](O)c2ccc(Cl)cc2)[C@@H](O)[C@H]1O] | 407.8 | 9-((2R,3R,4S,5R)-5-((R)-(4- chlorophenyl)(hydroxy)methyl)-3,4- dihydroxytetrahydrofuran-2-yl)- 3,9-dihydro- 6H-purin-6-one O-methyl oxime |
| 54 | 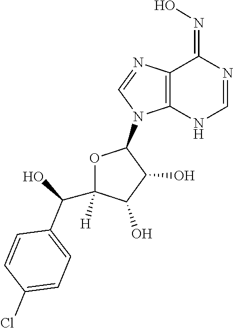 [SMILES: O/N=c1/nc[nH]c2c1ncn2[C@@H]1O[C@H]([C@H](O)c2ccc(Cl)cc2)[C@@H](O)[C@H]1O] | 393.8 | 9-((2R,3R,4S,5R)-5-((R)-(4- chlorophenyl)(hydroxy)methyl)-3,4- dihydroxytetrahydrofuran-2-yl)-3,9- dihydro-6H-purin-6-one oxime |
| 55 | 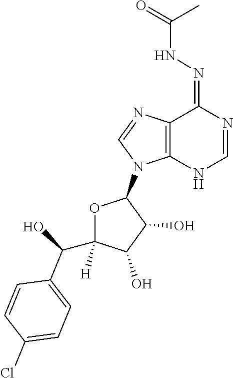 [SMILES: CC(=O)N/N=c1/nc[nH]c2c1ncn2[C@@H]1O[C@H]([C@H](O)c2ccc(Cl)cc2)[C@@H](O)[C@H]1O] | 434.8 | N′-(9-((2R,3R,4S,5R)-5-((R)-(4- chlorophenyl)(hydroxy)methyl)-3,4- dihydroxytetrahydrofuran-2-yl)-3,9- dihydro-6H-purin- 6-ylidene)acetohydrazide |
| 56 | 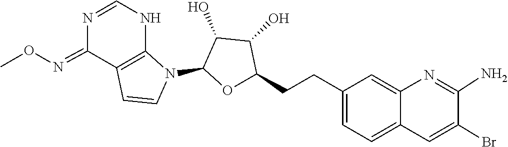 [SMILES: CO/N=c1\nc[nH]c2c1ccn2[C@@H]1O[C@H](CCc2ccc3cc(Br)c(N)nc3c2)[C@@H](O)[C@H]1O] | 515.4 | -7-((2R,3R,4S,5R)-5-(2-(2-amino-3- bromoquinolin-7-yl)ethyl)-3,4- dihydroxytetrahydrofuran-2-yl)- 1,7-dihydro- 4H-pyrrolo[2,3-d]pyrimidin- 4-one O-methyl oxime |
| 57 | 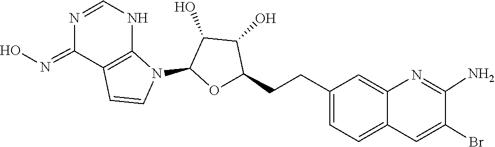 [SMILES: Nc1nc2cc(CC[C@H]3O[C@@H](n4ccc5/c(=N/O)nc[nH]c54)[C@H](O)[C@@H]3O)ccc2cc1Br] | 501.3 | 7-((2R,3R,4S,5R)-5-(2-(2-amino-3- bromoquinolin-7-yl)ethyl)-3,4- dihydroxytetrahydrofuran-2-yl)- 1,7-dihydro- 4H-pyrrolo[2,3-d]pyrimidin- 4-one oxime |
| 59 | 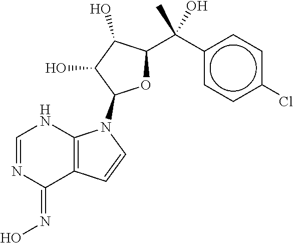 | 406.8 | 7-((2R,3R,4S,5S)-5-((R)-1- (4-chlorophenyl)-1- hydroxyethyl)-3,4- dihydroxytetrahydrofuran- 2-yl)-1,7-dihydro-4H- pyrrolo[2,3-d]pyrimidin- 4-one oxime |
| 60 | 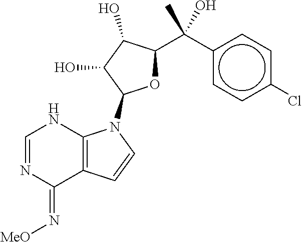 | 420.9 | 7-((2R,3R,4S,5S)-5-((R)-1- (4-chlorophenyl)-1- hydroxyethyl)-3,4- dihydroxytetrahydrofuran- 2-yl)-1,7-dihydro-4H- pyrrolo[2,3-d] pyrimidin-4- one O-methyl oxime |
| 61 | 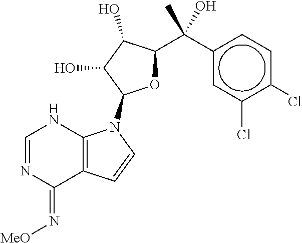 | 455.3 | 7-((2R,3R,4S,5S)-5-((R)-1- (3,4-dichlorophenyl)-1- hydroxyethyl)-3,4- dihydroxytetrahydrofuran- 2-yl)-1,7-dihydro-4H- pyrrolo[2,3-d]pyrimidin- 4-one O-methyl oxime |
| 62 | 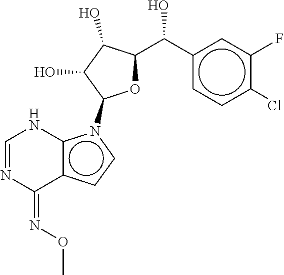 | 424.8 | 7-((2R,3R,4S,5R)-5-((R)-(4-chloro-3- fluorophenyl)(hydroxy)methyl)-3,4- dihydroxytetrahydrofuran-2-yl)-1,7- dihydro-4H- pyrrolo[2,3-d]pyrimidin- 4-one O-methyl oxime |
| 63 | 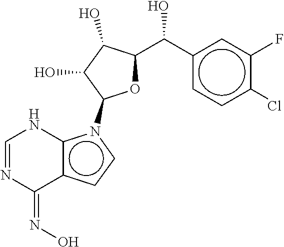 | 410.8 | 7-((2R,3R,4S,5R)-5-((R)-(4-chloro-3- fluorophenyl)(hydroxy)methyl)-3,4- dihydroxytetrahydrofuran-2-yl)-1,7- dihydro-4H-pyrrolo[2,3-d]pyrimidin- 4-one oxime |
| 64 | 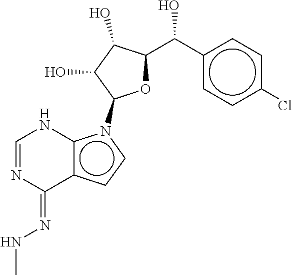 | 405.8 | (2R,3S,4R,5R)-2-((R)-(4- chlorophenyl)(hydroxy)methyl)- 5-((Z)-4-(2- methylhydrazineylidene)- 1,4-dihydro-7H- pyrrolo[2,3-d]pyrimidin-7- yl)tetrahydrofuran-3,4-diol |
| 65 | 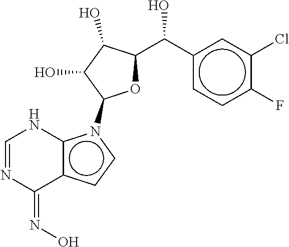 | 410.8 | 7-((2R,3R,4S,5R)-5-((R)- (3-chloro-4- fluorophenyl)(hydroxy)methyl)- 3,4-dihydroxytetrahydrofuran- 2-yl)-1,7-dihydro-4H- pyrrolo[2,3-d]pyrimidin- 4-one oxime |
| 66 | 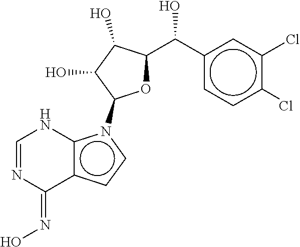 | 427.2 | 7-((2R,3R,4S,5R)-5-((R)-(3,4- dichlorophenyl)(hydroxy)methyl)- 3,4-dihydroxytetrahydrofuran- 2-yl)-1,7-dihydro-4H- pyrrolo[2,3-d]pyrimidin- 4-one oxime |
| 67 | 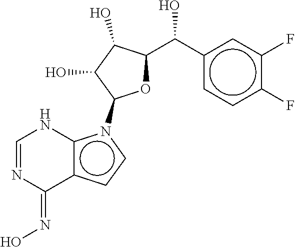 | 394.3 | 7-((2R,3R,4S,5R)-5-((R)-(3,4- difluorophenyl)(hydroxy)methyl)- 3,4-dihydroxytetrahydrofuran- 2-yl)-1,7-dihydro-4H- pyrrolo[2,3-d]pyrimidin- 4-one oxime |
| 68 | 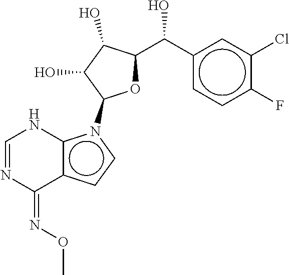 | 424.8 | 7-((2R,3R,4S,5R)-5- ((R)-(3-chloro-4- fluorophenyl)(hydroxy)methyl)- 3,4-dihydroxytetrahydrofuran- 2-yl)-1,7-dihydro- 4H-pyrrolo[2,3-d]pyrimidin- 4-one O-methyl oxime |
| 69 | 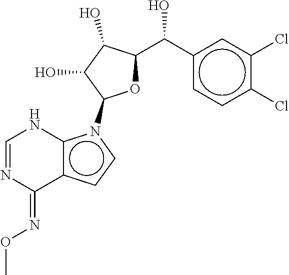 | 441.3 | 7-((2R,3R,4S,5R)-5-((R)-(3,4- dichlorophenyl)(hydroxy)methyl)- 3,4-dihydroxytetrahydrofuran- 2-yl)-1,7-dihydro-4H- pyrrolo[2,3-d]pyrimidin- 4-one O-methyl oxime |
| 70 | 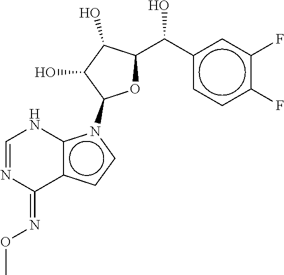 | 408.4 | 7-((2R,3R,4S,5R)-5-((R)-(3,4- difluorophenyl)(hydroxy)methyl)- 3,4-dihydroxytetrahydrofuran- 2-yl)-1,7-dihydro-4H- pyrrolo[2,3-d]pyrimidin- 4-one O-methyl oxime |
| 71 | 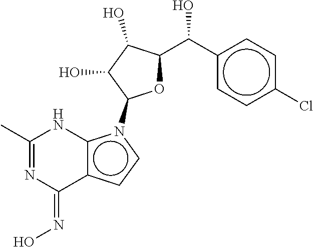 | 406.8 | 7-((2R,3R,4S,5R)-5-((R)-(4- chlorophenyl)(hydroxy)methyl)- 3,4-dihydroxytetrahydrofuran- 2-yl)-2-methyl-1,7-dihydro-4H- pyrrolo[2,3-d]pyrimidin- 4-one oxime |
| 72 | 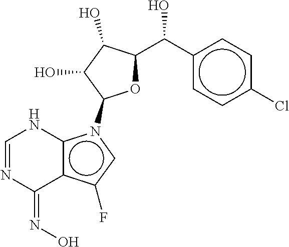 | 410.8 | 7-((2R,3R,4S,5R)-5-((R)-(4- chlorophenyl)(hydroxy)methyl)- 3,4-dihydroxytetrahydrofuran- 2-yl)-5-fluoro-1,7-dihydro-4H- pyrrolo[2,3-d]pyrimidin-4-one oxime |
| 73 | 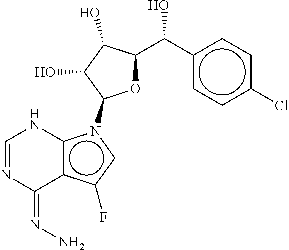 | 409.8 | (2R,3S,4R,5R)-2-((R)-(4- chlorophenyl)(hydroxy)methyl)- 5-(5-fluoro-4- hydrazineylidene-1,4-dihydro-7H- pyrrolo[2,3-d]pyrimidin-7- yl)tetrahydrofuran-3,4-diol |
| 74 | 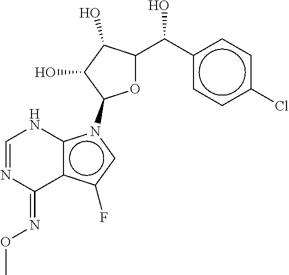 | 424.8 | 7-((2R,3R,4S)-5-((R)-(4- chlorophenyl)(hydroxy)methyl)- 3,4-dihydroxytetrahydrofuran- 2-yl)-5-fluoro-1,7-dihydro-4H- pyrrolo[2,3-d]pyrimidin- 4-one O-methyl oxime |
| 75 | 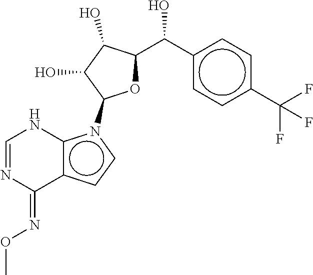 | 440.4 | 7-((2R,3R,4S,5R)-3,4-dihydroxy- 5-((R)- (trifluoromethyl)phenyl)methyl) tetrahydrofuran- 2-yl)-1,7-dihydro-4H-pyrrolo[2,3-d] pyrimidin-4-one O-methyl oxime |
| 76 | 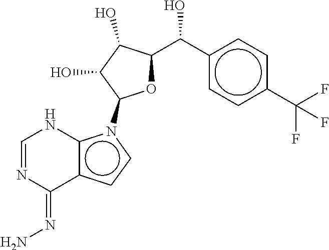 | 425.4 | (2R,3R,4S,5R)-2-(4-hydrazineylidene- 1,4-dihydro-7H- pyrrolo[2,3-d]pyrimidin-7-y1)-5- ((R)-hydroxy(4- (trifluoromethyl)phenyl)methyl) tetrahydrofuran-3,4-diol |
| 77 | 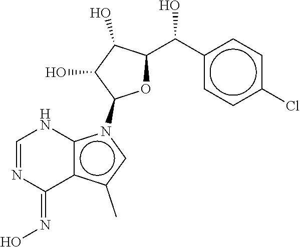 | 406.8 | 7-((2R,3R,4S,5R)-5-((R)-(4- chlorophenyl)(hydroxy)methyl)- 3,4-dihydroxytetrahydrofuran- 2-yl)-5-methyl-1,7-dihydro- 4H-pyrrolo[2,3-d]pyrimidin- 4-one oxime |
| 78 | 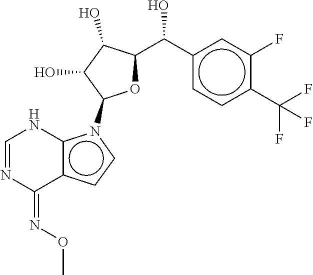 | 458.4 | 7-((2R,3R,4S,5R)-5-((R)- (3-fluoro-4- (trifluoromethyl)phenyl)(hydroxy) methyl)- 3,4-dihydroxytetrahydrofuran- 2-yl)-1,7- dihydro-4H- pyrrolo[2,3-d]pyrimidin- 4-one O-methyl oxime |
| 79 | 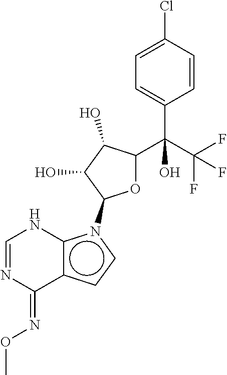 | 475.8 | 7-((2R,3R,4S)-5-((R)-1- (4-chlorophenyl)- 2,2,2-trifluoro-1-hydroxyethyl)-3,4- dihydroxytetrahydrofuran-2-yl)- 1,7-dihydro-4H- pyrrolo[2,3-d]pyrimidin- 4-one O-methyl oxime |
| 80 | 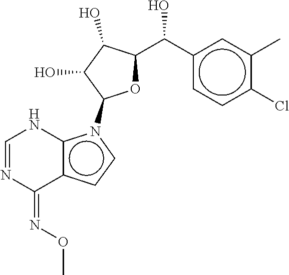 | 420.9 | 7-((2R,3R,4S,5R)-5-((R)-(4-chloro-3- methylphenyl)(hydroxy)methyl)-3,4- dihydroxytetrahydrofuran-2-yl)- 1,7-dihydro-4H- pyrrolo[2,3-d]pyrimidin- 4-one O-methyl oxime |
| 81 | 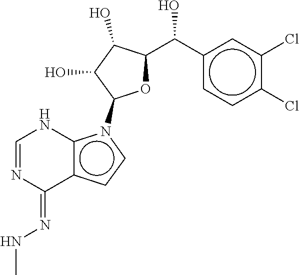 | 440.3 | (2R,3S,4R,5R)-2-((R)-(3,4- dichlorophenyl)(hydroxy)methyl)- 5-((2)-4-(2- methylhydrazineylidene)-1,4- dihydro-7H- pyrrolo[2,3-d]pyrimidin- 7-yl)tetrahydrofuran-3,4-diol |
| 82 | 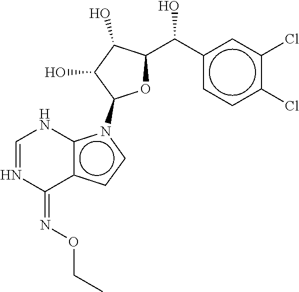 | 455.3 | 7-((2R,3R,4S,5R)-5-((R)-(3,4- dichlorophenyl)(hydroxy)methyl)- 3,4-dihydroxytetrahydrofuran- 2-yl)-3,7-dihydro- 4H-pyrrolo[2,3-d]pyrimidin-4- one O-ethyl oxime |
| 83 | 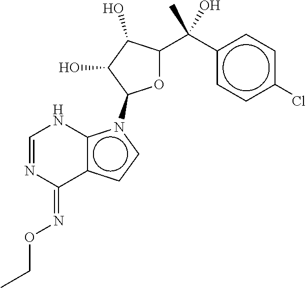 | 434.9 | 7-((2R,3R,4S)-5-((R)-1- (4-chlorophenyl)- 1-hydroxyethyl)-3,4- dihydroxytetrahydrofuran- 2-yl)-1,7-dihydro-4H- pyrrolo[2,3-d]pyrimidin- 4-one O-ethyl oxime |
| 84 | 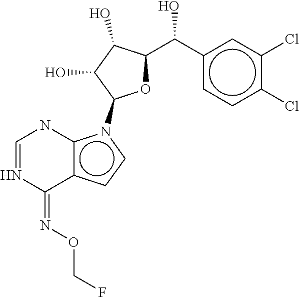 | 459.3 | 7-((2R,3R,4S,5R)-5-((R)-(3,4- dichlorophenyl)(hydroxy)methyl)- 3,4-dihydroxytetrahydrofuran- 2-yl)-3,7-dihydro-4H- pyrrolo[2,3-d]pyrimidin-4- one O-fluoromethyl oxime |
| 85 | 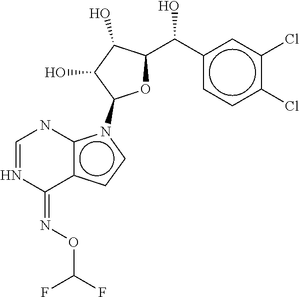 | 477.2 | 7-((2R,3R,4S,5R)-5-((R)-(3,4- dichlorophenyl)(hydroxy)methyl)- 3,4-dihydroxytetrahydrofuran- 2-yl)-3,7-dihydro-4H- pyrrolo[2,3-d]pyrimidin- 4-one O-difluoromethyl oxime |
| 86 | 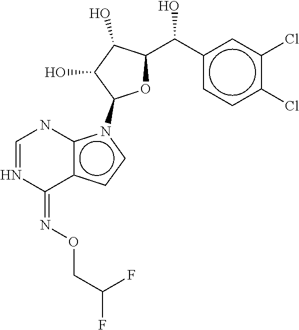 | 491.3 | 7-((2R,3R,4S,5R)-5-((R)-(3,4- dichlorophenyl)(hydroxy)methyl)- 3,4-dihydroxytetrahydrofuran- 2-yl)-3,7-dihydro-4H- pyrrolo[2,3-d]pyrimidin- 4-one O-(2,2-difluoroethyl) oxime |
| 87 | 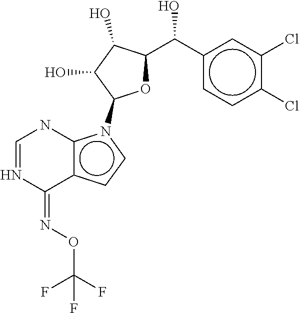 | 495.2 | 7-((2R,3R,4S,5R)-54(R)-(3,4- dichlorophenyl)(hydroxy)methyl)- 3,4-dihydroxytetrahydrofuran- 2-yl)-3,7-dihydro-4H- pyrrolo[2,3-d]pyrimidin- 4-one O-trifluoromethyl oxime |
| 88 | 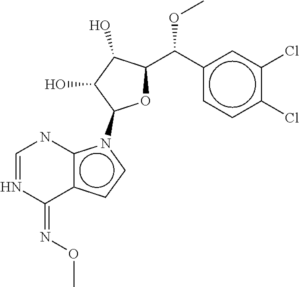 | 455.3 | 7-((2R,3R,4S,5R)-5-((R)-(3,4- dichlorophenyl)(methoxy)methyl)- 3,4-dihydroxytetrahydrofuran- 2-yl)-3,7-dihydro-4H- pyrrolo[2,3-d]pyrimidin- 4-one O-methyl oxime |
| 89 | 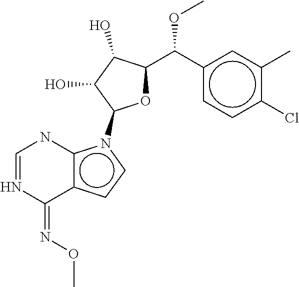 | 434.9 | 7-((2R,3R,4S,5R)-5-((R)-(4-chloro-3- methylphenyl)(methoxy)methyl)- 3,4-dihydroxytetrahydrofuran- 2-yl)-3,7-dihydro-4H- pyrrolo[2,3-d]pyrimidin- 4-one O-methyl oxime |
| 90 | 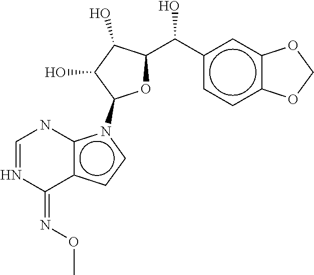 | 416.4 | 7-((2R,3R,4S,5R)-5-((R)- benzo[d][1,3]dioxol- 5-yl(hydroxy)methyl)-3,4- dihydroxytetrahydrofuran- 2-yl)-3,7-dihydro- 4H-pyrrolo[2,3-d]pyrimidin- 4-one O-methyl oxime |
| 91 | 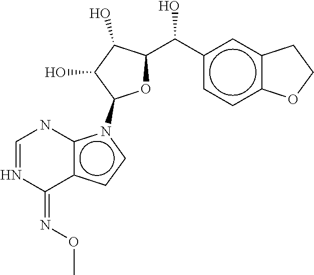 | 414.4 | 7-((2R,3R,4S,5R)-5-((R)-(2,3- dihydrobenzofuran-5- yl)(hydroxy)methyl)- 3,4-dihydroxytetrahydrofuran- 2-yl)-3,7-dihydro-4H- pyrrolo[2,3-d]pyrimidin- 4-one O-methyl oxime |
| 92 | 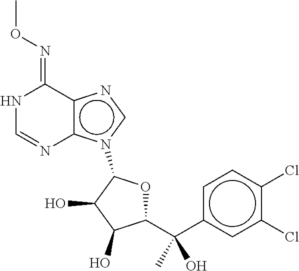 | 456.28 | (2)-9-((2R,3R,4S,5S)-5- ((R)-1-(3,4- dichlorophenyl)-1-hydroxyethyl)- 3,4-dihydroxytetrahydrofuran-2- yl)-1,9-dihydro- 6H-purin-6-one O-methyl oxime |
| 93 | 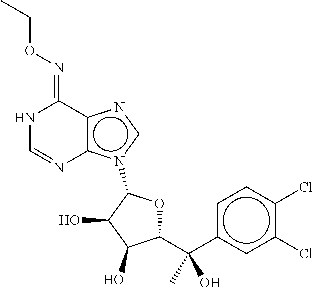 | 470.31 | (Z)-9-((2R,3R,4S,5S)-5-((R)-1-(3,4- dichlorophenyl)-1-hydroxyethyl)-3,4- dihydroxytetrahydrofuran-2-yl)-1,9- dihydro- 6H-purin-6-one O-ethyl oxime |
| 94 | 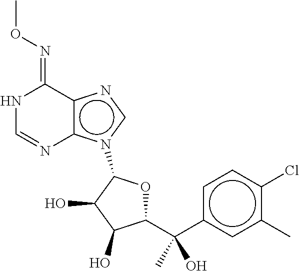 | 435.86 | (Z)-9-((2R,3R,4S,5S)-5- ((R)-1-(4-chloro-3- methylphenyl)-1-hydroxyethyl)- 3,4-dihydroxytetrahydrofuran- 2-yl)-1,9-dihydro- 6H-purin-6-one O-methyl oxime |
| 95 | 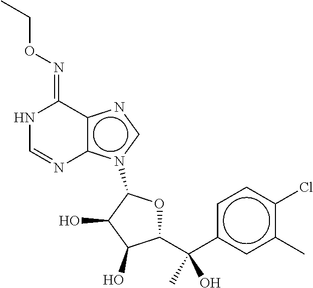 | 449.89 | (Z)-9-((2R,3R,4S,5S)-5-((R)- 1-(4-chloro-3-methylphenyl)-1- hydroxyethyl)-3,4- dihydroxytetrahydrofuran- 2-yl)-1,9-dihydro- 6H-purin-6-one O-ethyl oxime |
| 96 | 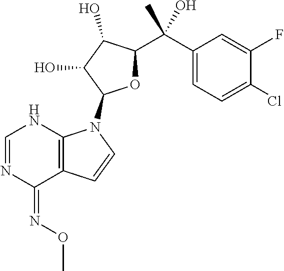 [SMILES: CO/N=c1/nc[nH]c2c1ccn2[C@@H]1O[C@H]([C@](C)(O)c2ccc(Cl)c(F)c2)[C@@H](O)[C@H]1O] | 438.84 | (E)-7-((2R,3R,4S,5S)-5-((R)- 1-(4-chloro-3-fluorophenyl)- 1-hydroxyethyl)-3,4- dihydroxytetrahydrofuran- 2-yl)-1,5,6,7- tetrahydro-4H- pyrrolo[2,3-d]pyrimidin- 4-one O-methyl oxime |
| 97 | 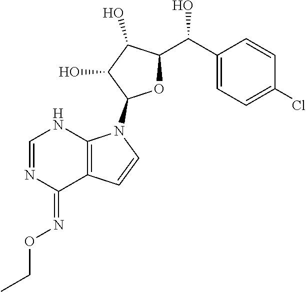 [SMILES: CCO/N=c1\nc[nH]c2c1ccn2[C@@H]1O[C@H]([C@H](O)c2ccc(Cl)cc2)[C@@H](O)[C@H]1O] | 420.85 | (Z)-7-((2R,3R,4S,5R)-5-((R)- (4-chlorophenyl)(hydroxy)methyl)- 3,4-dihydroxytetrahydrofuran- 2-yl)-1,7-dihydro-4H- pyrrolo[2,3-d]pyrimidin- 4-one O-ethyl oxime |
| 98 | 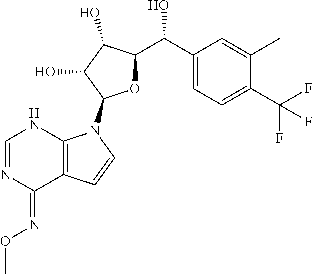 [SMILES: CO/N=c1\nc[nH]c2c1ccn2[C@@H]1O[C@H]([C@H](O)c2ccc(C(F)(F)F)c(C)c2)[C@@H](O)[C@H]1O] | 454.4 | (Z)-7-((2R,3R,4S,5R)- 3,4-dihydroxy-5-((R)- hydroxy(3-methyl-4- (trifluoromethyl)phenyl)methyl) tetrahydrofuran-2- yl)-1,7-dihydro-4H-pyrrolo[2,3-d] pyrimidin-4-one O-methyl oxime |
| 99 | 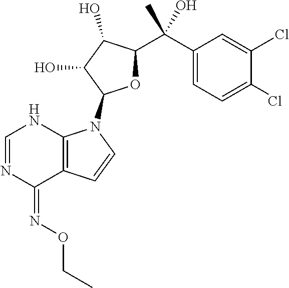 [SMILES: CCO/N=c1/nc[nH]c2c1ccn2[C@@H]1O[C@H]([C@](C)(O)c2ccc(Cl)c(Cl)c2)[C@@H](O)[C@H]1O] | 469.32 | (E)-7-((2R,3R,4S,5S)-5- ((R)-1-(3,4-dichlorophenyl)- 1-hydroxyethyl)-3,4- dihydroxytetrahydrofuran- 2-yl)-1,7-dihydro- 4H-pyrrolo[2,3-d]pyrimidin- 4-one O-ethyl oxime |
| 100 | 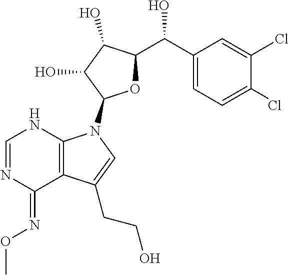 [SMILES: CO/N=c1\nc[nH]c2c1c(CCO)cn2[C@@H]1O[C@H]([C@H](O)c2ccc(Cl)c(Cl)c2)[C@@H](O)[C@H]1O] | 485.32 | (Z)-7-((2R,3R,4S,5R)-5- ((R)-(3,4- dichlorophenyl)(hydroxy)methyl)- 3,4-dihydroxytetrahydrofuran- 2-yl)-5-(2-hydroxyethyl)- 1,5-dihydro-4H-714- pyrrolo[2,3-d]pyrimidin- 4-one O-methyl oxime |
| 101 | 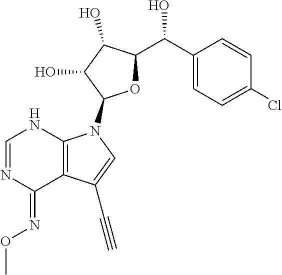 [SMILES: C#Cc1cn([C@@H]2O[C@H]([C@H](O)c3ccc(Cl)cc3)[C@@H](O)[C@H]2O)c2[nH]cn/c(=N\OC)c12] | 430.84 | (Z)-7-((2R,3R,4S,5R)-5-((R)-(4- chlorophenyl)(hydroxy)methyl)-3,4- dihydroxytetrahydrofuran-2-yl)-5- ethynyl-1,7-dihydro-4H- pyrrolo[2,3-d]pyrimidin- 4-one O-methyl oxime |
| 102 | 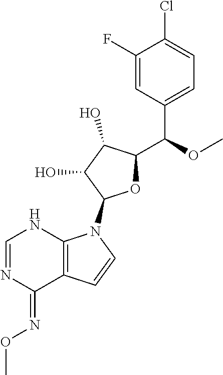 [SMILES: CO/N=c1\nc[nH]c2c1ccn2[C@@H]1O[C@H]([C@H](OC)c2ccc(Cl)c(F)c2)[C@@H](O)[C@H]1O] | 438.84 | (Z)-7-((2R,3R,4S,5S)-5- ((R)-(4-chloro-3- fluorophenyl)(methoxy)methyl)- 3,4-dihydroxytetrahydrofuran- 2-yl)-1,7-dihydro- 4H-pyrrolo[2,3-d]pyrimidin- 4-one O-methyl oxime |
| 103 | 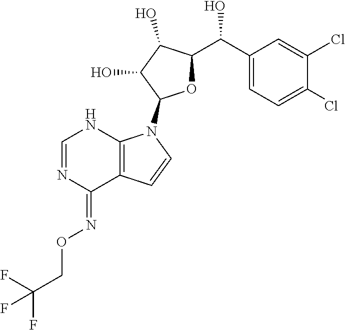 [SMILES: O[C@@H]1[C@H](O)[C@@H]([C@H](O)c2ccc(Cl)c(Cl)c2)O[C@H]1n1ccc2/c(=N/OCC(F)(F)F)nc[nH]c21] | 509.26 | (Z)-7-((2R,3R,4S,5R)-5- ((R)-(3,4- dichlorophenyl)(hydroxy)methyl)- 3,4-dihydroxytetrahydrofuran- 2-yl)-1,4a,7,7a- tetrahydro-4H- pyrrolo[2,3-d]pyrimidin-4- one O-(2,2,2-trifluoroethyl) oxime |
| 104 | 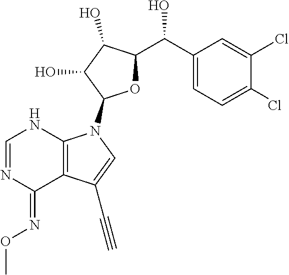 [SMILES: C#Cc1cn([C@@H]2O[C@H]([C@H](O)c3ccc(Cl)c(Cl)c3)[C@@H](O)[C@H]2O)c2[nH]cn/c(=N\OC)c12] | 465.29 | (Z)-7-((2R,3R,4S,5R)-5- ((R)-(3,4- dichlorophenyl)(hydroxy)methyl)- 3,4-dihydroxytetrahydrofuran- 2-yl)-5-ethynyl-1,5-dihydro-4H- 714-pyrrolo[2,3-d]pyrimidin- 4-one O-methyl oxime |
| 105 | 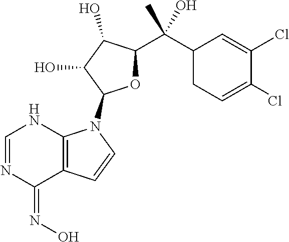 [SMILES: C[C@@](O)(C1C=C(Cl)C(Cl)=CC1)[C@H]1O[C@@H](n2ccc3/c(=N\O)nc[nH]c32)[C@H](O)[C@@H]1O] | 441.27 | (E)-7-((2R,3R,4S,5S)-5- ((1R)-1-(3,4- dichlorocyclohexa-2,4- dien-1-yl)-1- hydroxyethyl)-3,4- dihydroxytetrahydrofuran- 2-yl)-1,7-dihydro-4H- pyrrolo[2,3-d]pyrimidin- 4-one oxime |
| 106 | 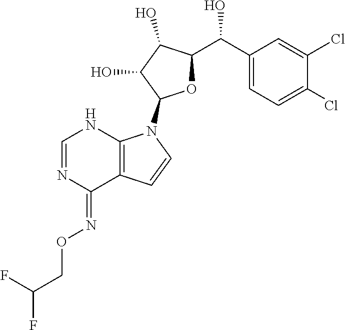 [SMILES: O[C@@H]1[C@H](O)[C@@H]([C@H](O)c2ccc(Cl)c(Cl)c2)O[C@H]1n1ccc2/c(=N/OCC(F)F)nc[nH]c21] | 491.27 | (Z)-7-((2R,3R,45,5R)-5- ((R)-(3,4- dichlorophenyl)(hydroxy)methyl)- 3,4-dihydroxytetrahydrofuran- 2-yl)-1,4a,7,7a- tetrahydro-4H- pyrrolo[2,3-d]pyrimidin-4- one O-(2,2-difluoroethyl) oxime |
| 107 | 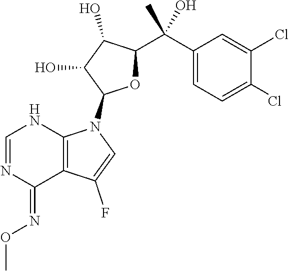 [SMILES: CO/N=c1\nc[nH]c2c1c(F)cn2[C@@H]1O[C@H]([C@](C)(O)c2ccc(Cl)c(Cl)c2)[C@@H](O)[C@H]1O] | 473.28 | (Z)-7-((2R,3R,4S,5S)-5- ((1)-1-(3,4- dichlorophenyl)-1-hydroxyethyl)- 3,4-dihydroxytetrahydrofuran- 2-yl)-5-fluoro-1,7-dihydro-4H- pyrrolo[2,3-d]pyrimidin- 4-one O-methyl oxime |
| 108 | 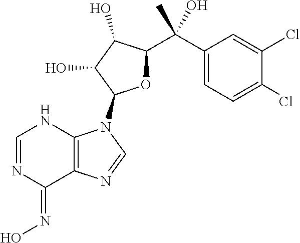 [SMILES: C[C@@](O)(c1ccc(Cl)c(Cl)c1)[C@H]1O[C@@H](n2cnc3/c(=N/O)nc[nH]c32)[C@H](O)[C@@H]1O] | 442.25 | (Z)-9-((2R,3R,4S,5S)-5- ((1)-1-(3,4- dichlorophenyl)-1- hydroxyethyl)-3,4- dihydroxytetrahydrofuran- 2-yl)-3,9-dihydro-6H-purin- 6-one oxime |

Compounds of the disclosure of Formula III and Formula IV include, for example, the compounds identified in Table B.

TABLE B

| Ex. | | | |
|---|---|---|---|
| No. | Structure | MW | Chemical Name |
| 1-B | 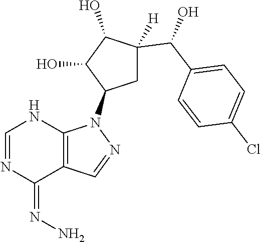 [SMILES: N/N=c1/nc[nH]c2c1cnn2[C@@H]1C[C@H]([C@H](O)c2ccc(Cl)cc2)[C@@H](O)[C@H]1O] | 390.828 | (1S,2R,3R,5R)-3-((S)-(4- chlorophenyl)(hydroxy)methyl)-5- ((E)-4-hydrazineylidene-4,7- dihydro-1H-pyrazolo[3,4- d]pyrimidin-1-yl)cyclopentane-1,2- diol |
| 2-B | 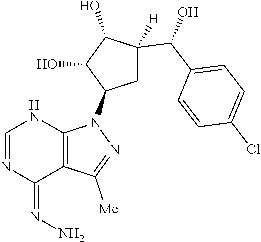 [SMILES: Cc1nn([C@@H]2C[C@H]([C@H](O)c3ccc(Cl)cc3)[C@@H](O)[C@H]2O)c2[nH]cn/c(=N/N)c12] | 404.855 | (1S,2R,3R,5R)-3-((S)-(4- chlorophenyl)(hydroxy)methyl)-5- ((E)-4-hydrazineylidene-3-methyl- 4,7-dihydro-1H-pyrazolo[3,4- d]pyrimidin-1-yl)cyclopentane-1,2- diol |
| 3-B | 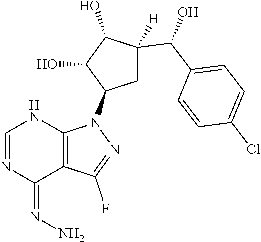 [SMILES: N/N=c1/nc[nH]c2c1c(F)nn2[C@@H]1C[C@H]([C@H](O)c2ccc(Cl)cc2)[C@@H](O)[C@H]1O] | 408.8184 | (1S,2R,3R,5R)-3-((S)-(4- chlorophenyl)(hydroxy)methyl)-5- ((E)-3-fluoro-4-hydrazineylidene- 4,7-dihydro-1H-pyrazolo[3,4- d]pyrimidin-1-yl)cyclopentane-1,2- diol |
| 4-B | 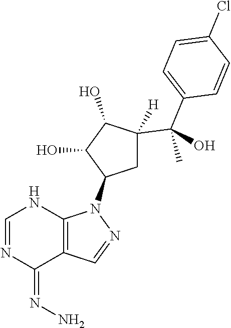 [SMILES: C[C@@](O)(c1ccc(Cl)cc1)[C@H]1C[C@@H](n2ncc3/c(=N\N)nc[nH]c32)[C@H](O)[C@@H]1O] | 404.855 | (1S,2R,3S,5R)-3-((S)-1-(4- chlorophenyl)-1-hydroxyethyl)-5- ((E)-4-hydrazineylidene-4,7- dihydro-1H-pyrazolo[3,4- d]pyrimidin-1-yl)cyclopentane-1,2- diol |
| 5-B | 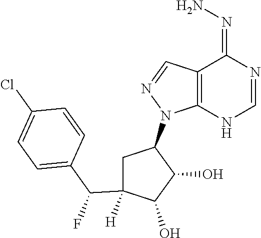 [SMILES: N/N=c1/nc[nH]c2c1cnn2[C@@H]1C[C@H]([C@H](F)c2ccc(Cl)cc2)[C@@H](O)[C@H]1O] | 392.8194 | (1S,2R,3S,5R)-3-((S)-(4- chlorophenyl)fluoromethyl)-5-((E)- 4-hydrazineylidene-4,7-dihydro-1H- pyrazolo[3,4-d]pyrimidin-1- yl)cyclopentane-1,2-diol |
| 6-B | 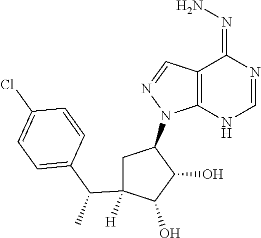 [SMILES: C[C@H](c1ccc(Cl)cc1)[C@H]1C[C@@H](n2ncc3/c(=N\N)nc[nH]c32)[C@H](O)[C@@H]1O] | 388.856 | (1S,2R,3R,5R)-3-((S)-1-(4- chlorophenyl)ethyl)-5-((E)-4- hydrazineylidene-4,7-dihydro-1H- pyrazolo[3,4-d]pyrimidin-1- yl)cyclopentane-1,2-diol |
| 7-B | 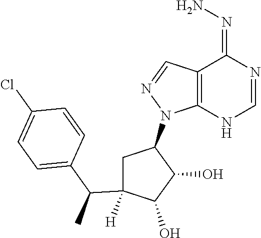 [SMILES: C[C@@H](c1ccc(Cl)cc1)[C@H]1C[C@@H](n2ncc3/c(=N\N)nc[nH]c32)[C@H](O)[C@@H]1O] | 388.856 | (1S,2R,3R,5R)-3-((R)-1-(4- chlorophenyl)ethyl)-5-((E)-4- hydrazineylidene-4,7-dihydro-1H- pyrazolo[3,4-d]pyrimidin-1- yl)cyclopentane-1,2-diol |
| 8-B | 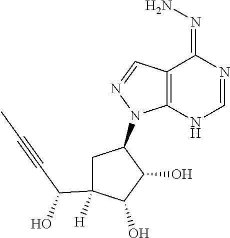 [SMILES: CC#C[C@@H](O)[C@H]1C[C@@H](n2ncc3/c(=N\N)nc[nH]c32)[C@H](O)[C@@H]1O] | 318.337 | (1R,2S,3R,5R)-3-((E)-4- hydrazineylidene-4,7-dihydro-1H- pyrazolo[3,4-d]pyrimidin-1-yl)-5- ((S)-1-hydroxybut-2-yn-1- yl)cyclopentane-1,2-diol |
| 9-B | 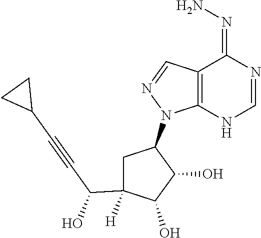 [SMILES: N/N=c1/nc[nH]c2c1cnn2[C@@H]1C[C@H]([C@H](O)C#CC2CC2)[C@@H](O)[C@H]1O] | 344.375 | (1S,2R,3R,5R)-3-((S)-3-cyclopropyl- 1-hydroxyprop-2-yn-1-yl)-5-((E)-4- hydrazineylidene-4,7-dihydro-1H- pyrazolo[3,4-d]pyrimidin-1- yl)cyclopentane-1,2-diol |
| 10-B | 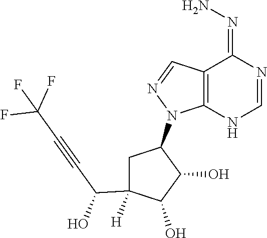 [SMILES: N/N=c1/nc[nH]c2c1cnn2[C@@H]1C[C@H]([C@H](O)C#CC(F)(F)F)[C@@H](O)[C@H]1O] | 372.3082 | (1R,2S,3R,5R)-3-((E)-4- hydrazineylidene-4,7-dihydro-1H- pyrazolo[3,4-d]pyrimidin-1-yl)-5- ((S)-4,4,4-trifluoro-1-hydroxybut-2- yn-1-yl)cyclopentane-1,2-diol |
| 11-B | 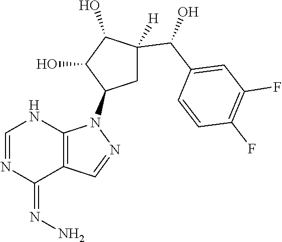 [SMILES: N/N=c1/nc[nH]c2c1cnn2[C@@H]1C[C@H]([C@H](O)c2ccc(F)c(F)c2)[C@@H](O)[C@H]1O] | 392.3668 | (1S,2R,3R,5R)-3-((S)-(3,4- difluorophenyl)(hydroxy)methyl)-5- ((E)-4-hydrazineylidene-4,7- dihydro-1H-pyrazolo[3,4- d]pyrimidin-1-yl)cyclopentane-1,2- diol |
| 12-B | 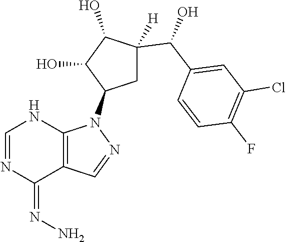 [SMILES: N/N=c1/nc[nH]c2c1cnn2[C@@H]1C[C@H]([C@H](O)c2ccc(F)c(Cl)c2)[C@@H](O)[C@H]1O] | 408.8184 | (1S,2R,3R,5R)-3-((S)-(3-chloro-4- fluorophenyl)(hydroxy)methyl)-5- ((E)-4-hydrazineylidene-4,7- dihydro-1H-pyrazolo[3,4- d]pyrimidin-1-yl)cyclopentane-1,2- diol |
| 13-B | 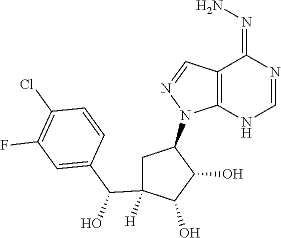 [SMILES: N/N=c1/nc[nH]c2c1cnn2[C@@H]1C[C@H]([C@H](O)c2ccc(Cl)c(F)c2)[C@@H](O)[C@H]1O] | 408.8184 | (1S,2R,3R,5R)-3-((S)-(4-chloro-3- fluorophenyl)(hydroxy)methyl)-5- ((E)-4-hydrazineylidene-4,7- dihydro-1H-pyrazolo[3,4- d]pyrimidin-1-yl)cyclopentane-1,2- diol |
| 14-B | 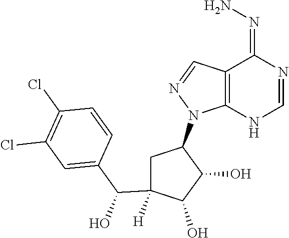 [SMILES: N/N=c1/nc[nH]c2c1cnn2[C@@H]1C[C@H]([C@H](O)c2ccc(Cl)c(Cl)c2)[C@@H](O)[C@H]1O] | 425.27 | (1S,2R,3R,5R)-3-((S)-(3,4- dichlorophenyl)(hydroxy)methyl)-5- ((E)-4-hydrazineylidene-4,7- dihydro-1H-pyrazolo[3,4- d]pyrimidin-1-yl)cyclopentane-1,2- diol |
| 15-B | 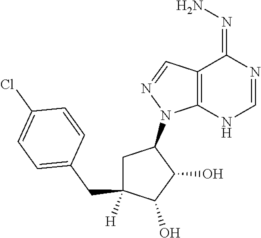 [SMILES: N/N=c1/nc[nH]c2c1cnn2[C@@H]1C[C@H](Cc2ccc(Cl)cc2)[C@@H](O)[C@H]1O] | 374.829 | (1S,2R,3S,5R)-3-(4-chlorobenzyl)-5- ((E)-4-hydrazineylidene-4,7- dihydro-1H-pyrazolo[3,4- d]pyrimidin-1-yl)cyclopentane-1,2- diol |
| 16-B | 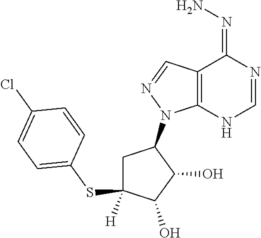 [SMILES: N/N=c1/nc[nH]c2c1cnn2[C@@H]1C[C@H](Sc2ccc(Cl)cc2)[C@@H](O)[C@H]1O] | 392.862 | (1S,2S,3S,5R)-3-((4- chlorophenyl)thio)-5-((E)-4- hydrazineylidene-4,7-dihydro-1H- pyrazolo[3,4-d]pyrimidin-1- yl)cyclopentane-1,2-diol |
| 17-B | 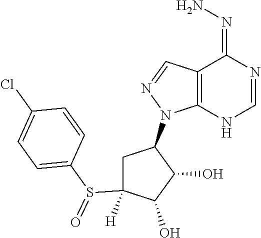 [SMILES: N/N=c1/nc[nH]c2c1cnn2[C@@H]1C[C@H](S(=O)c2ccc(Cl)cc2)[C@@H](O)[C@H]1O] | 408.861 | (1S,2S,3S,5R)-3-((4- chlorophenyl)sulfinyl)-5-((E)-4- hydrazineylidene-4,7-dihydro-1H- pyrazolo[3,4-d]pyrimidin-1- yl)cyclopentane-1,2-diol |
| 18-B | 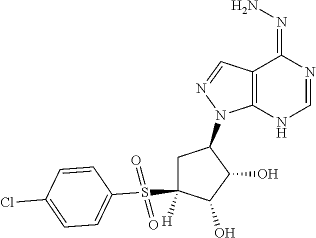 [SMILES: N/N=c1/nc[nH]c2c1cnn2[C@@H]1C[C@H](S(=O)(=O)c2ccc(Cl)cc2)[C@@H](O)[C@H]1O] | 424.86 | (1S,2S,3S,5R)-3-((4- chlorophenyl)sulfonyl)-5-((E)-4- hydrazineylidene-4,7-dihydro-1H- pyrazolo[3,4-d]pyrimidin-1- yl)cyclopentane-1,2-diol |
| 19-B | 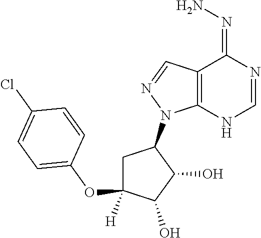 [SMILES: N/N=c1/nc[nH]c2c1cnn2[C@@H]1C[C@H](Oc2ccc(Cl)cc2)[C@@H](O)[C@H]1O] | 376.801 | (1S,2S,3S,5R)-3-(4-chlorophenoxy)- 5-((E)-4-hydrazineylidene-4,7- dihydro-1H-pyrazolo[3,4- d]pyrimidin-1-yl)cyclopentane-1,2- diol |
| 20-B | 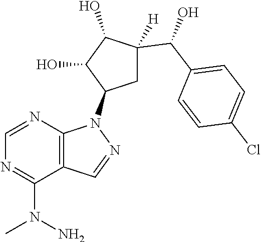 [SMILES: CN(N)c1ncnc2c1cnn2[C@@H]1C[C@H]([C@H](O)c2ccc(Cl)cc2)[C@@H](O)[C@H]1O] | 404.855 | (1S,2R,3R,5R)-3-((S)-(4- chlorophenyl)(hydroxy)methyl)-5- (4-(1-methylhydrazineyl)-1H- pyrazolo[3,4-d]pyrimidin-1- yl)cyclopentane-1,2-diol |
| 21-B | 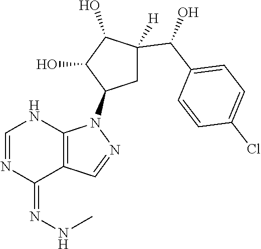 [SMILES: CN/N=c1/nc[nH]c2c1cnn2[C@@H]1C[C@H]([C@H](O)c2ccc(Cl)cc2)[C@@H](O)[C@H]1O] | 404.855 | (1S,2R,3R,5R)-3-((S)-(4- chlorophenyl)(hydroxy)methyl)-5- ((E)-4-(2-methylhydrazineylidene)- 4,7-dihydro-1H-pyrazolo[3,4- d]pyrimidin-1-yl)cyclopentane-1,2- diol |
| 22-B | 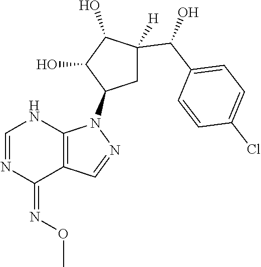 [SMILES: CO/N=c1/nc[nH]c2c1cnn2[C@@H]1C[C@H]([C@H](O)c2ccc(Cl)cc2)[C@@H](O)[C@H]1O] | 405.839 | (E)-1-((1R,2S,3R,4R)-4-((S)-(4- chlorophenyl)(hydroxy)methyl)-2,3- dihydroxycyclopentyl)-1,7-dihydro- 4H-pyrazolo[3,4-d]pyrimidin-4-one O-methyl oxime |
| 23-B | 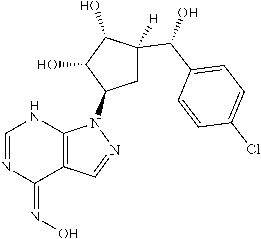 [SMILES: O/N=c1/nc[nH]c2c1cnn2[C@@H]1C[C@H]([C@H](O)c2ccc(Cl)cc2)[C@@H](O)[C@H]1O] | 391.812 | (E)-1-((1R,2S,3R,4R)-4-((S)-(4- chlorophenyl)(hydroxy)methyl)-2,3- dihydroxycyclopentyl)-1,7-dihydro- 4H-pyrazolo[3,4-d]pyrimidin-4-one oxime |
| 24-B | 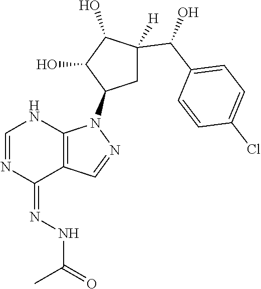 [SMILES: CC(=O)N/N=c1/nc[nH]c2c1cnn2[C@@H]1C[C@H]([C@H](O)c2ccc(Cl)cc2)[C@@H](O)[C@H]1O] | 432.865 | N′-((E)-1-((1R,2S,3R,4R)-4-((S)-(4- chlorophenyl)(hydroxy)methyl)-2,3- dihydroxycyclopentyl)-1,7-dihydro- 4H-pyrazolo[3,4-d]pyrimidin-4- ylidene)acetohydrazide |
| 25-B | 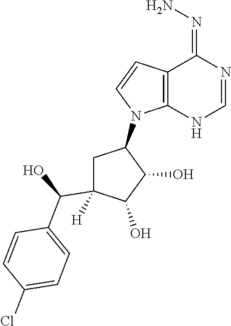 [SMILES: N/N=c1/nc[nH]c2c1ccn2[C@@H]1C[C@H]([C@H](O)c2ccc(Cl)cc2)[C@@H](O)[C@H]1O] | 389.84 | (1S,2R,3R,5R)-3-((S)-(4- chlorophenyl)(hydroxy)methyl)-5- ((E)-4-hydrazineylidene-1,4- dihydro-7H-pyrrolo[2,3- d]pyrimidin-7-yl)cyclopentane-1,2- diol |
| 26-B | 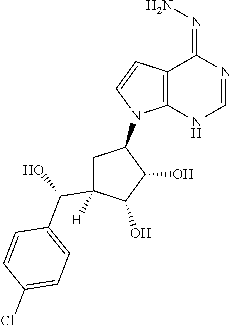 [SMILES: N/N=c1/nc[nH]c2c1ccn2[C@@H]1C[C@H]([C@@H](O)c2ccc(Cl)cc2)[C@@H](O)[C@H]1O] | 389.84 | (1S,2R,3R,5R)-3-((R)-(4- chlorophenyl)(hydroxy)methyl)-5- ((E)-4-hydrazineylidene-1,4- dihydro-7H-pyrrolo[2,3- d]pyrimidin-7-yl)cyclopentane-1,2- diol |
| 27-B | 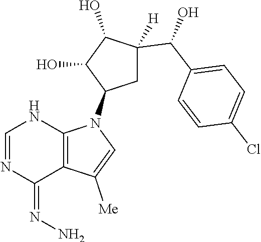 [SMILES: Cc1cn([C@@H]2C[C@H]([C@H](O)c3ccc(Cl)cc3)[C@@H](O)[C@H]2O)c2[nH]cn/c(=N/N)c12] | 403.867 | (1S,2R,3R,5R)-3-((S)-(4- chlorophenyl)(hydroxy)methyl)-5- ((E)-4-hydrazineylidene-5-methyl- 1,4-dihydro-7H-pyrrolo[2,3- d]pyrimidin-7-yl)cyclopentane-1,2- diol |
| 28-B | 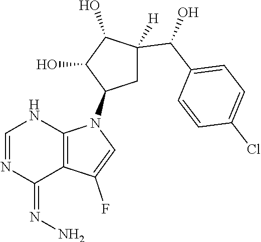 [SMILES: N/N=c1/nc[nH]c2c1c(F)cn2[C@@H]1C[C@H]([C@H](O)c2ccc(Cl)cc2)[C@@H](O)[C@H]1O] | 407.8304 | (1S,2R,3R,5R)-3-((S)-(4- chlorophenyl)(hydroxy)methyl)-5- ((E)-5-fluoro-4-hydrazineylidene- 1,4-dihydro-7H-pyrrolo[2,3- d]pyrimidin-7-yl)cyclopentane-1,2- diol |
| 29-B | 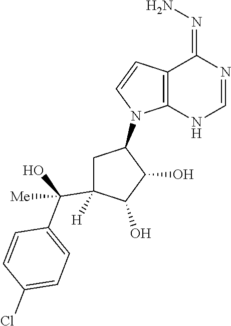 [SMILES: C[C@@](O)(c1ccc(Cl)cc1)[C@H]1C[C@@H](n2ccc3/c(=N\N)nc[nH]c32)[C@H](O)[C@@H]1O] | 403.867 | (1S,2R,3S,5R)-3-((S)-1-(4- chlorophenyl)-1-hydroxyethyl)-5- ((E)-4-hydrazineylidene-1,4- dihydro-7H-pyrrolo[2,3- d]pyrimidin-7-yl)cyclopentane-1,2- diol |
| 30-B | 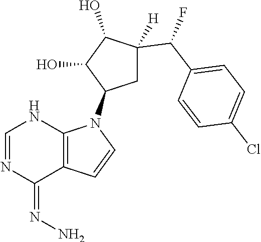 [SMILES: N/N=c1/nc[nH]c2c1ccn2[C@@H]1C[C@H]([C@H](F)c2ccc(Cl)cc2)[C@@H](O)[C@H]1O] | 391.8314 | (1S,2R,3S,5R)-3-((S)-(4- chlorophenyl)fluoromethyl)-5-((E)- 4-hydrazineylidene-1,4-dihydro-7H- pyrrolo[2,3-d]pyrimidin-7- yl)cyclopentane-1,2-diol |
| 31-B | 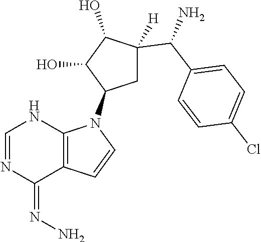 [SMILES: N/N=c1/nc[nH]c2c1ccn2[C@@H]1C[C@H]([C@H](N)c2ccc(Cl)cc2)[C@@H](O)[C@H]1O] | 388.856 | (1S,2R,3R,5R)-3-((S)-amino(4- chlorophenyl)methyl)-5-((E)-4- hydrazineylidene-1,4-dihydro-7H- pyrrolo[2,3-d]pyrimidin-7- yl)cyclopentane-1,2-diol |
| 32-B | 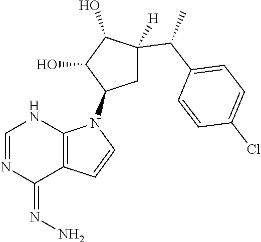 [SMILES: C[C@H](c1ccc(Cl)cc1)[C@H]1C[C@@H](n2ccc3/c(=N\N)nc[nH]c32)[C@H](O)[C@@H]1O] | 387.868 | (1S,2R,3R,5R)-3-((S)-1-(4- chlorophenyl)ethyl)-5-((E)-4- hydrazineylidene-1,4-dihydro-7H- pyrrolo[2,3-d]pyrimidin-7- yl)cyclopentane-1,2-diol |
| 33-B | 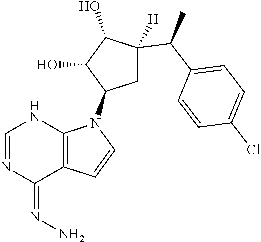 [SMILES: C[C@@H](c1ccc(Cl)cc1)[C@H]1C[C@@H](n2ccc3/c(=N\N)nc[nH]c32)[C@H](O)[C@@H]1O] | 387.868 | (1S,2R,3R,5R)-3-((R)-1-(4- chlorophenyl)ethyl)-5-((E)-4- hydrazineylidene-1,4-dihydro-7H- pyrrolo[2,3-d]pyrimidin-7- yl)cyclopentane-1,2-diol |
| 34-B | 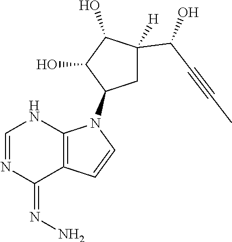 [SMILES: CC#C[C@@H](O)[C@H]1C[C@@H](n2ccc3/c(=N\N)nc[nH]c32)[C@H](O)[C@@H]1O] | 317.349 | (1R,2S,3R,5R)-3-((E)-4- hydrazineylidene-1,4-dihydro-7H- pyrrolo[2,3-d]pyrimidin-7-yl)-5-((S)- 1-hydroxybut-2-yn-1- yl)cyclopentane-1,2-diol |
| 35-B | 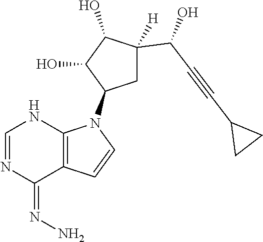 [SMILES: N/N=c1/nc[nH]c2c1ccn2[C@@H]1C[C@H]([C@H](O)C#CC2CC2)[C@@H](O)[C@H]1O] | 343.387 | (1S,2R,3R,5R)-3-((S)-3-cyclopropyl- 1-hydroxyprop-2-yn-1-yl)-5-((E)-4- hydrazineylidene-1,4-dihydro-7H- pyrrolo[2,3-d]pyrimidin-7- yl)cyclopentane-1,2-diol |
| 36-B | 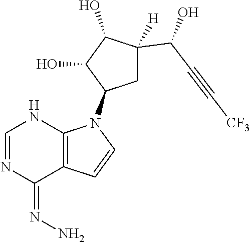 [SMILES: N/N=c1/nc[nH]c2c1ccn2[C@@H]1C[C@H]([C@H](O)C#CC(F)(F)F)[C@@H](O)[C@H]1O] | 371.3202 | (1R,2S,3R,5R)-3-((E)-4- hydrazineylidene-1,4-dihydro-7H- pyrrolo[2,3-d]pyrimidin-7-yl)-5-((S)- 4,4,4-trifluoro-1-hydroxybut-2-yn-1- yl)cyclopentane-1,2-diol |
| 37-B | 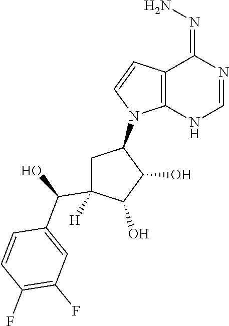 [SMILES: N/N=c1/nc[nH]c2c1ccn2[C@@H]1C[C@H]([C@H](O)c2ccc(F)c(F)c2)[C@@H](O)[C@H]1O] | 391.3788 | (1S,2R,3R,5R)-3-((S)-(3,4- difluorophenyl)(hydroxy)methyl)-5- ((E)-4-hydrazineylidene-1,4- dihydro-7H-pyrrolo[2,3- d]pyrimidin-7-yl)cyclopentane-1,2- diol |
| 38-B | 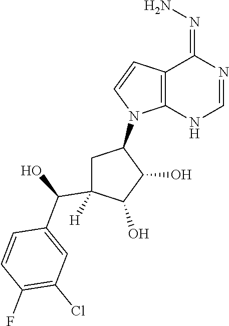 [SMILES: N/N=c1/nc[nH]c2c1ccn2[C@@H]1C[C@H]([C@H](O)c2ccc(F)c(Cl)c2)[C@@H](O)[C@H]1O] | 407.8304 | (1S,2R,3R,5R)-3-((S)-(3-chloro-4- fluorophenyl)(hydroxy)methyl)-5- ((E)-4-hydrazineylidene-1,4- dihydro-7H-pyrrolo[2,3- d]pyrimidin-7-yl)cyclopentane-1,2- diol |
| 39-B | 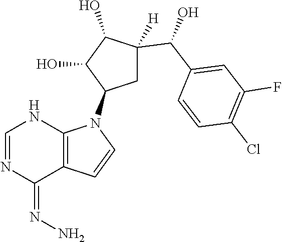 [SMILES: N/N=c1/nc[nH]c2c1ccn2[C@@H]1C[C@H]([C@H](O)c2ccc(Cl)c(F)c2)[C@@H](O)[C@H]1O] | 407.8304 | (1S,2R,3R,5R)-3-((S)-(4-chloro-3- fluorophenyl)(hydroxy)methyl)-5- ((E)-4-hydrazineylidene-1,4- dihydro-7H-pyrrolo[2,3- d]pyrimidin-7-yl)cyclopentane-1,2- diol |
| 40-B | 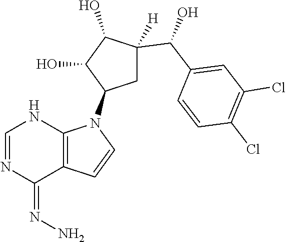 [SMILES: N/N=c1/nc[nH]c2c1ccn2[C@@H]1C[C@H]([C@H](O)c2ccc(Cl)c(Cl)c2)[C@@H](O)[C@H]1O] | 424.282 | (1S,2R,3R,5R)-3-((S)-(3,4- dichlorophenyl)(hydroxy)methyl)-5- ((E)-4-hydrazineylidene-1,4- dihydro-7H-pyrrolo[2,3- d]pyrimidin-7-yl)cyclopentane-1,2- diol |
| 41-B | 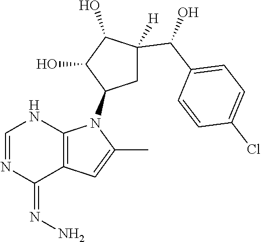 [SMILES: Cc1cc2/c(=N\N)nc[nH]c2n1[C@@H]1C[C@H]([C@H](O)c2ccc(Cl)cc2)[C@@H](O)[C@H]1O] | 403.867 | (1S,2R,3R,5R)-3-((S)-(4- chlorophenyl)(hydroxy)methyl)-5- ((E)-4-hydrazineylidene-6-methyl- 1,4-dihydro-7H-pyrrolo[2,3- d]pyrimidin-7-yl)cyclopentane-1,2- diol |
| 42-B | 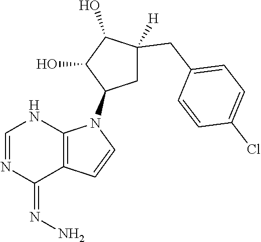 [SMILES: N/N=c1/nc[nH]c2c1ccn2[C@@H]1C[C@H](Cc2ccc(Cl)cc2)[C@@H](O)[C@H]1O] | 373.841 | (1S,2R,3S,5R)-3-(4-chlorobenzyl)-5- ((E)-4-hydrazineylidene-1,4- dihydro-7H-pyrrolo[2,3- d]pyrimidin-7-yl)cyclopentane-1,2- diol |
| 43-B | 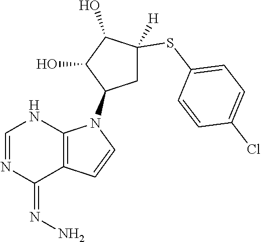 [SMILES: N/N=c1/nc[nH]c2c1ccn2[C@@H]1C[C@H](Sc2ccc(Cl)cc2)[C@@H](O)[C@H]1O] | 391.874 | (1S,2S,3S,5R)-3-((4- chlorophenyl)thio)-5-((E)-4- hydrazineylidene-1,4-dihydro-7H- pyrrolo[2,3-d]pyrimidin-7- yl)cyclopentane-1,2-diol |
| 44-B | 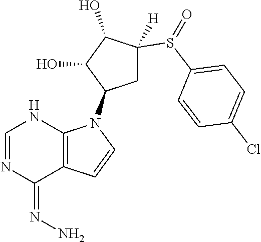 [SMILES: N/N=c1/nc[nH]c2c1ccn2[C@@H]1C[C@H](S(=O)c2ccc(Cl)cc2)[C@@H](O)[C@H]1O] | 407.873 | (1S,2S,3S,5R)-3-((4- chlorophenyl)sulfinyl)-5-((E)-4- hydrazineylidene-1,4-dihydro-7H- pyrrolo[2,3-d]pyrimidin-7- yl)cyclopentane-1,2-diol |
| 45-B | 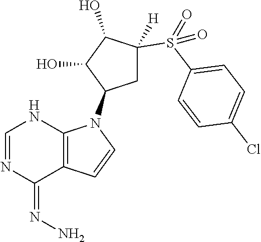 [SMILES: N/N=c1/nc[nH]c2c1ccn2[C@@H]1C[C@H](S(=O)(=O)c2ccc(Cl)cc2)[C@@H](O)[C@H]1O] | 423.872 | (1S,2S,3S,5R)-3-((4- chlorophenyl)sulfonyl)-5-((E)-4- hydrazineylidene-1,4-dihydro-7H- pyrrolo[2,3-d]pyrimidin-7- yl)cyclopentane-1,2-diol |
| 46-B | 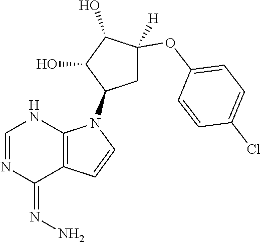 [SMILES: N/N=c1/nc[nH]c2c1ccn2[C@@H]1C[C@H](Oc2ccc(Cl)cc2)[C@@H](O)[C@H]1O] | 375.813 | (1S,2S,3S,5R)-3-(4-chlorophenoxy)- 5-((E)-4-hydrazineylidene-1,4- dihydro-7H-pyrrolo[2,3- d]pyrimidin-7-yl)cyclopentane-1,2- diol |
| 47-B | 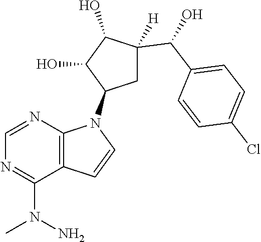 [SMILES: CN(N)c1ncnc2c1ccn2[C@@H]1C[C@H]([C@H](O)c2ccc(Cl)cc2)[C@@H](O)[C@H]1O] | 403.867 | (1S,2R,3R,5R)-3-((S)-(4- chlorophenyl)(hydroxy)methyl)-5- (4-(1-methylhydrazineyl)-7H- pyrrolo[2,3-d]pyrimidin-7- yl)cyclopentane-1,2-diol |
| 48-B | 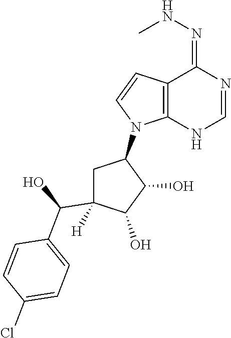 [SMILES: CN/N=c1/nc[nH]c2c1ccn2[C@@H]1C[C@H]([C@H](O)c2ccc(Cl)cc2)[C@@H](O)[C@H]1O] | 403.867 | (1S,2R,3R,5R)-3-((S)-(4- chlorophenyl)(hydroxy)methyl)-5- ((E)-4-(2-methylhydrazineylidene)- 1,4-dihydro-7H-pyrrolo[2,3- d]pyrimidin-7-yl)cyclopentane-1,2- diol |
| 49-B | 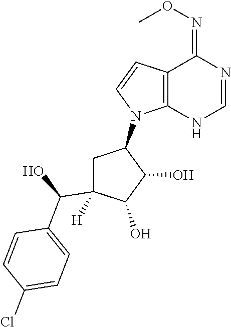 [SMILES: CO/N=c1/nc[nH]c2c1ccn2[C@@H]1C[C@H]([C@H](O)c2ccc(Cl)cc2)[C@@H](O)[C@H]1O] | 404.851 | (E)-7-((1R,2S,3R,4R)-4-((S)-(4- chlorophenyl)(hydroxy)methyl)-2,3- dihydroxycyclopentyl)-1,7-dihydro- 4H-pyrrolo[2,3-d]pyrimidin-4-one O-methyl oxime |
| 50-B | 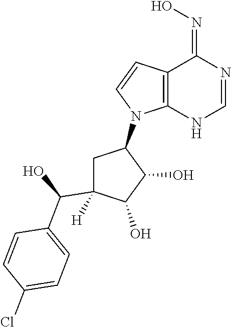 [SMILES: O/N=c1/nc[nH]c2c1ccn2[C@@H]1C[C@H]([C@H](O)c2ccc(Cl)cc2)[C@@H](O)[C@H]1O] | 390.824 | (E)-7-((1R,2S,3R,4R)-4-((S)-(4- chlorophenyl)(hydroxy)methyl)-2,3- dihydroxycyclopentyl)-1,7-dihydro- 4H-pyrrolo[2,3-d]pyrimidin-4-one oxime |
| 51-B | 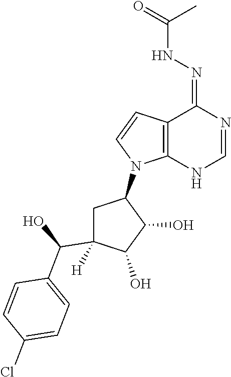 [SMILES: CC(=O)N/N=c1/nc[nH]c2c1ccn2[C@@H]1C[C@H]([C@H](O)c2ccc(Cl)cc2)[C@@H](O)[C@H]1O] | 431.877 | N′-((E)-7-((1R,2S,3R,4R)-4-((S)-(4- chlorophenyl)(hydroxy)methyl)-2,3- dihydroxycyclopentyl)-1,7-dihydro- 4H-pyrrolo[2,3-d]pyrimidin-4- ylidene)acetohydrazide |
| 52-B | 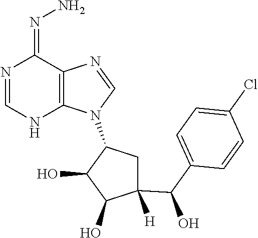 [SMILES: N/N=c1/nc[nH]c2c1ncn2[C@@H]1C[C@H]([C@H](O)c2ccc(Cl)cc2)[C@@H](O)[C@H]1O] | 390.828 | (1S,2R,3R,5R)-3-((S)-(4- chlorophenyl)(hydroxy)methyl)-5- ((E)-6-hydrazineylidene-3,6- dihydro-9H-purin-9- yl)cyclopentane-1,2-diol |
| 53-B | 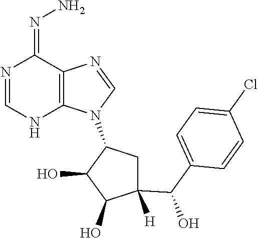 [SMILES: N/N=c1/nc[nH]c2c1ncn2[C@@H]1C[C@H]([C@@H](O)c2ccc(Cl)cc2)[C@@H](O)[C@H]1O] | 390.828 | (1S,2R,3R,5R)-3-((R)-(4- chlorophenyl)(hydroxy)methyl)-5- ((E)-6-hydrazineylidene-3,6- dihydro-9H-purin-9- yl)cyclopentane-1,2-diol |
| 54-B | 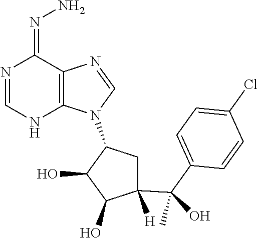 [SMILES: C[C@@](O)(c1ccc(Cl)cc1)[C@H]1C[C@@H](n2cnc3/c(=N\N)nc[nH]c32)[C@H](O)[C@@H]1O] | 404.855 | (1S,2R,3S,5R)-3-((S)-1-(4- chlorophenyl)-1-hydroxyethyl)-5- ((E)-6-hydrazineylidene-3,6- dihydro-9H-purin-9- yl)cyclopentane-1,2-diol |
| 55-B | 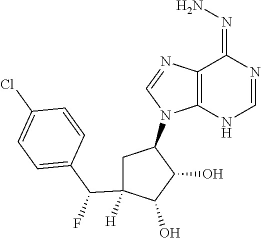 [SMILES: N/N=c1/nc[nH]c2c1ncn2[C@@H]1C[C@H]([C@H](F)c2ccc(Cl)cc2)[C@@H](O)[C@H]1O] | 392.8194 | (1S,2R,3S,5R)-3-((S)-(4- chlorophenyl)fluoromethyl)-5-((E)- 6-hydrazineylidene-3,6-dihydro-9H- purin-9-yl)cyclopentane-1,2-diol |
| 56-B | 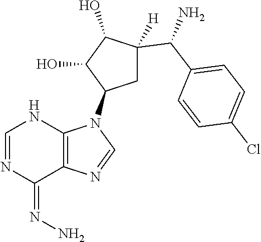 [SMILES: N/N=c1/nc[nH]c2c1ncn2[C@@H]1C[C@H]([C@H](N)c2ccc(Cl)cc2)[C@@H](O)[C@H]1O] | 389.844 | (1S,2R,3R,5R)-3-((S)-amino(4- chlorophenyl)methyl)-5-((E)-6- hydrazineylidene-3,6-dihydro-9H- purin-9-yl)cyclopentane-1,2-diol |
| 57-B | 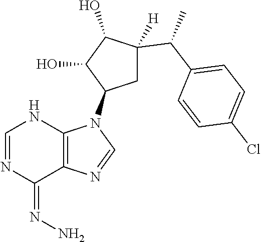 [SMILES: C[C@H](c1ccc(Cl)cc1)[C@H]1C[C@@H](n2cnc3/c(=N\N)nc[nH]c32)[C@H](O)[C@@H]1O] | 388.856 | (1S,2R,3R,5R)-3-((S)-1-(4- chlorophenyl)ethyl)-5-((E)-6- hydrazineylidene-3,6-dihydro-9H- purin-9-yl)cyclopentane-1,2-diol |
| 58-B | 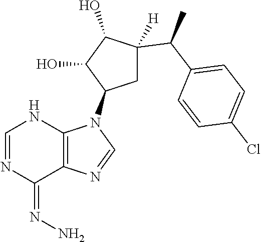 [SMILES: C[C@@H](c1ccc(Cl)cc1)[C@H]1C[C@@H](n2cnc3/c(=N\N)nc[nH]c32)[C@H](O)[C@@H]1O] | 388.856 | (1S,2R,3R,5R)-3-((R)-1-(4- chlorophenyl)ethyl)-5-((E)-6- hydrazineylidene-3,6-dihydro-9H- purin-9-yl)cyclopentane-1,2-diol |
| 59-B | 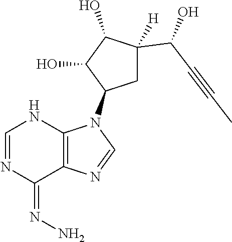 [SMILES: CC#C[C@@H](O)[C@H]1C[C@@H](n2cnc3/c(=N\N)nc[nH]c32)[C@H](O)[C@@H]1O] | 318.337 | (1R,2S,3R,5R)-3-((E)-6- hydrazineylidene-3,6-dihydro-9H- purin-9-yl)-5-((S)-1-hydroxybut-2- yn-1-yl)cyclopentane-1,2-diol |
| 60-B | 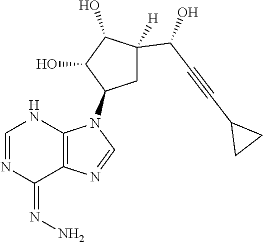 [SMILES: N/N=c1/nc[nH]c2c1ncn2[C@@H]1C[C@H]([C@H](O)C#CC2CC2)[C@@H](O)[C@H]1O] | 344.375 | (1S,2R,3R,5R)-3-((S)-3-cyclopropyl- 1-hydroxyprop-2-yn-1-yl)-5-((E)-6- hydrazineylidene-3,6-dihydro-9H- purin-9-yl)cyclopentane-1,2-diol |
| 61-B | 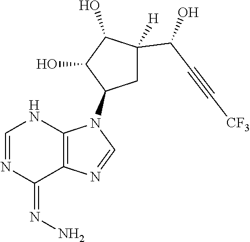 [SMILES: N/N=c1/nc[nH]c2c1ncn2[C@@H]1C[C@H]([C@H](O)C#CC(F)(F)F)[C@@H](O)[C@H]1O] | 372.3082 | (1R,2S,3R,5R)-3-((E)-6- hydrazineylidene-3,6-dihydro-9H- purin-9-yl)-5-((S)-4,4,4-trifluoro-1- hydroxybut-2-yn-1-yl)cyclopentane- 1,2-diol |
| 62-B | 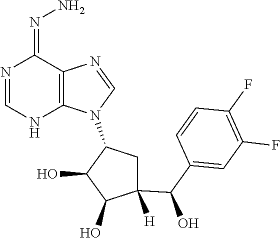 [SMILES: N/N=c1/nc[nH]c2c1ncn2[C@@H]1C[C@H]([C@H](O)c2ccc(F)c(F)c2)[C@@H](O)[C@H]1O] | 392.3668 | (1S,2R,3R,5R)-3-((S)-(3,4- difluorophenyl)(hydroxy)methyl)-5- ((E)-6-hydrazineylidene-3,6- dihydro-9H-purin-9- yl)cyclopentane-1,2-diol |
| 63-B | 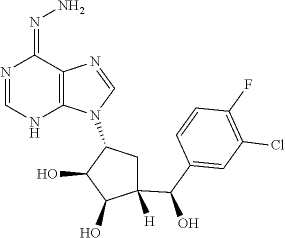 [SMILES: N/N=c1/nc[nH]c2c1ncn2[C@@H]1C[C@H]([C@H](O)c2ccc(F)c(Cl)c2)[C@@H](O)[C@H]1O] | 408.8184 | (1S,2R,3R,5R)-3-((S)-(3-chloro-4- fluorophenyl)(hydroxy)methyl)-5- ((E)-6-hydrazineylidene-3,6- dihydro-9H-purin-9- yl)cyclopentane-1,2-diol |
| 64-B | 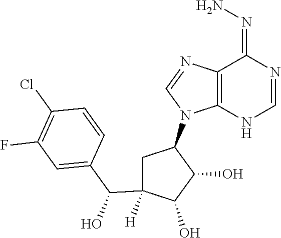 [SMILES: N/N=c1/nc[nH]c2c1ncn2[C@@H]1C[C@H]([C@H](O)c2ccc(Cl)c(F)c2)[C@@H](O)[C@H]1O] | 408.8184 | (1S,2R,3R,5R)-3-((S)-(4-chloro-3- fluorophenyl)(hydroxy)methyl)-5- ((E)-6-hydrazineylidene-3,6- dihydro-9H-purin-9- yl)cyclopentane-1,2-diol |
| 65-B | 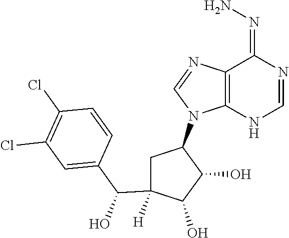 [SMILES: N/N=c1/nc[nH]c2c1ncn2[C@@H]1C[C@H]([C@H](O)c2ccc(Cl)c(Cl)c2)[C@@H](O)[C@H]1O] | 425.27 | (1S,2R,3R,5R)-3-((S)-(3,4- dichlorophenyl)(hydroxy)methyl)-5- ((E)-6-hydrazineylidene-3,6- dihydro-9H-purin-9- yl)cyclopentane-1,2-diol |
| 66-B | 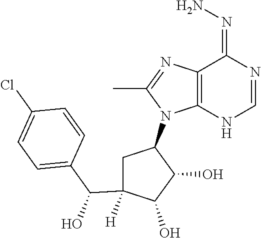 [SMILES: Cc1nc2/c(=N\N)nc[nH]c2n1[C@@H]1C[C@H]([C@H](O)c2ccc(Cl)cc2)[C@@H](O)[C@H]1O] | 404.855 | (1S,2R,3R,5R)-3-((S)-(4- chlorophenyl)(hydroxy)methyl)-5- ((E)-6-hydrazineylidene-8-methyl- 3,6-dihydro-9H-purin-9- yl)cyclopentane-1,2-diol |
| 67-B | 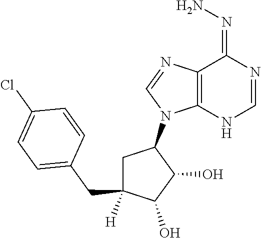 [SMILES: N/N=c1/nc[nH]c2c1ncn2[C@@H]1C[C@H](Cc2ccc(Cl)cc2)[C@@H](O)[C@H]1O] | 374.829 | (1S,2R,3S,5R)-3-(4-chlorobenzyl)-5- ((E)-6-hydrazineylidene-3,6- dihydro-9H-purin-9- yl)cyclopentane-1,2-diol |
| 68-B | 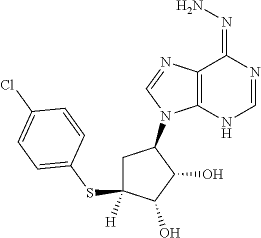 [SMILES: N/N=c1/nc[nH]c2c1ncn2[C@@H]1C[C@H](Sc2ccc(Cl)cc2)[C@@H](O)[C@H]1O] | 392.862 | (1S,2S,3S,5R)-3-((4- chlorophenyl)thio)-5-((E)-6- hydrazineylidene-3,6-dihydro-9H- purin-9-yl)cyclopentane-1,2-diol |
| 69-B | 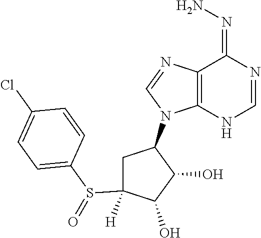 [SMILES: N/N=c1/nc[nH]c2c1ncn2[C@@H]1C[C@H](S(=O)c2ccc(Cl)cc2)[C@@H](O)[C@H]1O] | 408.861 | (1S,2S,3S,5R)-3-((4- chlorophenyl)sulfinyl)-5-((E)-6- hydrazineylidene-3,6-dihydro-9H- purin-9-yl)cyclopentane-1,2-diol |
| 70-B | 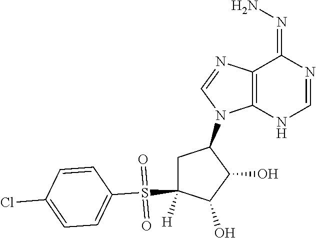 [SMILES: N/N=c1/nc[nH]c2c1ncn2[C@@H]1C[C@H](S(=O)(=O)c2ccc(Cl)cc2)[C@@H](O)[C@H]1O] | 424.86 | (1S,2S,3S,5R)-3-((4- chlorophenyl)sulfonyl)-5-((E)-6- hydrazineylidene-3,6-dihydro-9H- purin-9-yl)cyclopentane-1,2-diol |
| 71-B | 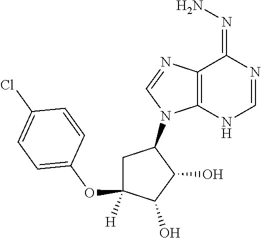 [SMILES: N/N=c1/nc[nH]c2c1ncn2[C@@H]1C[C@H](Oc2ccc(Cl)cc2)[C@@H](O)[C@H]1O] | 376.801 | (1S,2S,3S,5R)-3-(4-chlorophenoxy)- 5-((E)-6-hydrazineylidene-3,6- dihydro-9H-purin-9- yl)cyclopentane-1,2-diol |
| 72-B | 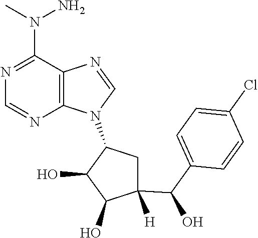 [SMILES: CN(N)c1ncnc2c1ncn2[C@@H]1C[C@H]([C@H](O)c2ccc(Cl)cc2)[C@@H](O)[C@H]1O] | 404.855 | (1S,2R,3R,5R)-3-((S)-(4- chlorophenyl)(hydroxy)methyl)-5- (6-(1-methylhydrazineyl)-9H-purin- 9-yl)cyclopentane-1,2-diol |
| 73-B | 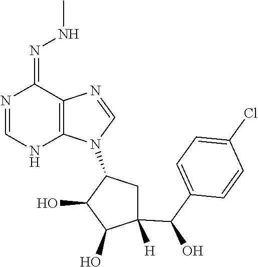 [SMILES: CN/N=c1/nc[nH]c2c1ncn2[C@@H]1C[C@H]([C@H](O)c2ccc(Cl)cc2)[C@@H](O)[C@H]1O] | 404.855 | (1S,2R,3R,5R)-3-((S)-(4- chlorophenyl)(hydroxy)methyl)-5- (6-(1-methylhydrazineyl)-9H-purin- 9-yl)cyclopentane-1,2-diol |
| 74-B | 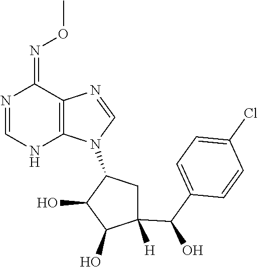 [SMILES: CO/N=c1/nc[nH]c2c1ncn2[C@@H]1C[C@H]([C@H](O)c2ccc(Cl)cc2)[C@@H](O)[C@H]1O] | 405.839 | (E)-9-((1R,2S,3R,4R)-4-((S)-(4- chlorophenyl)(hydroxy)methyl)-2,3- dihydroxycyclopentyl)-3,9-dihydro- 6H-purin-6-one O-methyl oxime |
| 75-B | 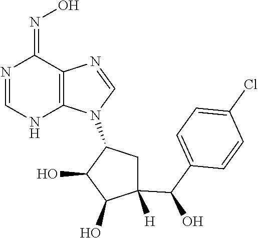 [SMILES: O/N=c1/nc[nH]c2c1ncn2[C@@H]1C[C@H]([C@H](O)c2ccc(Cl)cc2)[C@@H](O)[C@H]1O] | 391.812 | (E)-9-((1R,2S,3R,4R)-4-((S)-(4- chlorophenyl)(hydroxy)methyl)-2,3- dihydroxycyclopentyl)-3,9-dihydro- 6H-purin-6-one oxime |
| 76-B | 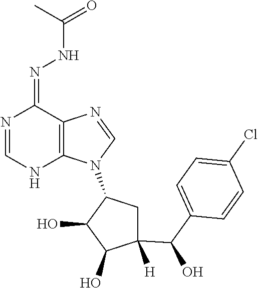 [SMILES: CC(=O)N/N=c1/nc[nH]c2c1ncn2[C@@H]1C[C@H]([C@H](O)c2ccc(Cl)cc2)[C@@H](O)[C@H]1O] | 432.865 | N′-((E)-9-((1R,2S,3R,4R)-4-((S)-(4- chlorophenyl)(hydroxy)methyl)-2,3- dihydroxycyclopentyl)-3,9-dihydro- 6H-purin-6-ylidene)acetohydrazide |
| 77-B | 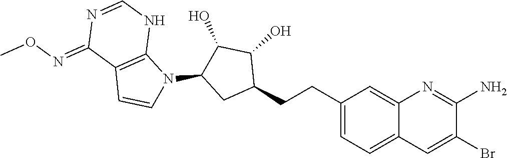 [SMILES: CO/N=c1\nc[nH]c2c1ccn2[C@@H]1C[C@H](CCc2ccc3cc(Br)c(N)nc3c2)[C@@H](O)[C@H]1O] | 513.40 | (Z)-7-((1R,2S,3R,4S)-4-(2-(2-amino- 3-bromoquinolin-7-yl)ethyl)-2,3- dihydroxycyclopentyl)-1,7-dihydro- 4H-pyrrolo[2,3-d]pyrimidin-4-one O-methyl oxime |
| 78-B | 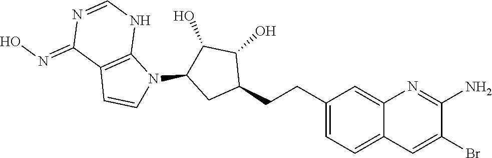 [SMILES: Nc1nc2cc(CC[C@H]3C[C@@H](n4ccc5/c(=N/O)nc[nH]c54)[C@H](O)[C@@H]3O)ccc2cc1Br] | 499.37 | (Z)-7-((1R,2S,3R,4S)-4-(2-(2-amino- 3-bromoquinolin-7-yl)ethyl)-2,3- dihydroxycyclopentyl)-1,7-dihydro- 4H-pyrrolo[2,3-d]pyrimidin-4-one oxime |
| 79-B | 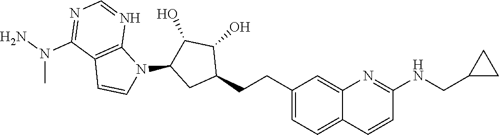 [SMILES: CN(N)c1ncnc2c1ccn2[C@@H]1C[C@H](CCc2ccc3ccc(NCC4CC4)nc3c2)[C@@H](O)[C@H]1O] | 487.61 | (1S,2R,3S,5R)-3-(2-(2- ((cyclopropylmethyl)amino)quinolin- 7-yl)ethyl)-5-(4-(1- methylhydrazineyl)-7H-pyrrolo[2,3- d]pyrimidin-7-yl)cyclopentane-1,2- diol |
| 80-B | 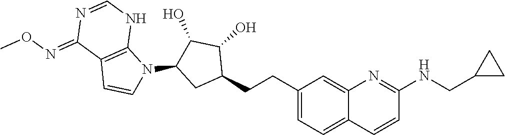 [SMILES: CO/N=c1\nc[nH]c2c1ccn2[C@@H]1C[C@H](CCc2ccc3ccc(NCC4CC4)nc3c2)[C@@H](O)[C@H]1O] | 488.59 | (Z)-7-((1R,2S,3R,4S)-4-(2-(2- ((cyclopropylmethyl)amino)quinolin- 7-yl)ethyl)-2,3- dihydroxycyclopentyl)-1,7-dihydro- 4H-pyrrolo[2,3-d]pyrimidin-4-one O-methyl oxime |
| 81-B | 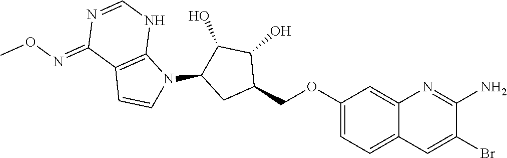 [SMILES: CO/N=c1\nc[nH]c2c1ccn2[C@@H]1C[C@H](COc2ccc3cc(Br)c(N)nc3c2)[C@@H](O)[C@H]1O] | 515.37 | (Z)-7-((1R,2S,3R,4R)-4-(((2-amino-3- bromoquinolin-7-yl)oxy)methyl)- 2,3-dihydroxycyclopentyl)-1,7- dihydro-4H-pyrrolo[2,3- d]pyrimidin-4-one O-methyl oxime |
| 82-B | 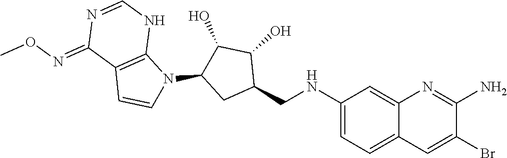 [SMILES: CO/N=c1\nc[nH]c2c1ccn2[C@@H]1C[C@H](CNc2ccc3cc(Br)c(N)nc3c2)[C@@H](O)[C@H]1O] | 514.38 | (Z)-7-((1R,2S,3R,4R)-4-(((2-amino-3- bromoquinolin-7-yl)amino)methyl)- 2,3-dihydroxycyclopentyl)-1,7- dihydro-4H-pyrrolo[2,3- d]pyrimidin-4-one O-methyl oxime |
| 83-B | 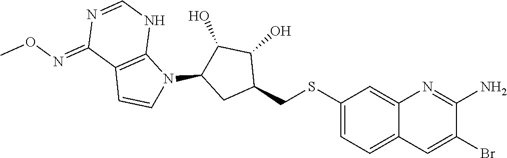 [SMILES: CO/N=c1\nc[nH]c2c1ccn2[C@@H]1C[C@H](CSc2ccc3cc(Br)c(N)nc3c2)[C@@H](O)[C@H]1O] | 531.43 | (Z)-7-((1R,2S,3R,4S)-4-(((2-amino-3- bromoquinolin-7-yl)thio)methyl)- 2,3-dihydroxycyclopentyl)-1,7- dihydro-4H-pyrrolo[2,3- d]pyrimidin-4-one O-methyl oxime |
| 84-B | 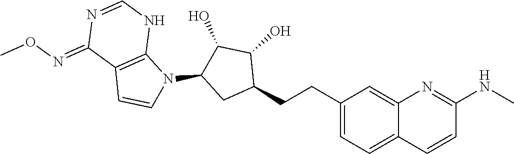 | 448.53 | (Z)-7-((1R,2S,3R,4S)-2,3-dihydroxy- 4-(2-(2-(methylamino)quinolin-7- yl)ethyl)cyclopentyl)-1,7-dihydro- 4H-pyrrolo[2,3-d]pyrimidin-4-one O-methyl oxime |
| 85-B | 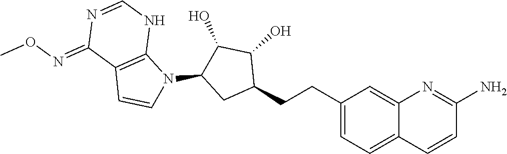 [SMILES: CO/N=c1\nc[nH]c2c1ccn2[C@@H]1C[C@H](CCc2ccc3ccc(N)nc3c2)[C@@H](O)[C@H]1O] | 434.5 | (Z)-7-((1R,2S,3R,4S)-4-(2-(2- aminoquinolin-7-yl)ethyl)-2,3- dihydroxycyclopentyl)-1,7-dihydro- 4H-pyrrolo[2,3-d]pyrimidin-4-one O-methyl oxime |
| 86-B | 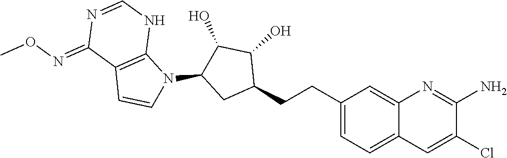 [SMILES: CO/N=c1\nc[nH]c2c1ccn2[C@@H]1C[C@H](CCc2ccc3cc(Cl)c(N)nc3c2)[C@@H](O)[C@H]1O] | 468.94 | (Z)-7-((1R,2S,3R,4S)-4-(2-(2-amino- 3-chloroquinolin-7-yl)ethyl)-2,3- dihydroxycyclopentyl)-1,7-dihydro- 4H-pyrrolo[2,3-d]pyrimidin-4-one O-methyl oxime |
| 87-B | 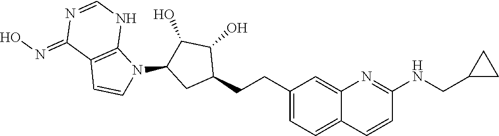 [SMILES: O/N=c1\nc[nH]c2c1ccn2[C@@H]1C[C@H](CCc2ccc3ccc(NCC4CC4)nc3c2)[C@@H](O)[C@H]1O] | 474.57 | (Z)-7-((1R,2S,3R,4S)-4-(2-(2- ((cyclopropylmethyl)amino)quinolin- 7-yl)ethyl)-2,3- dihydroxycyclopentyl)-1,7-dihydro- 4H-pyrrolo[2,3-d]pyrimidin-4-one oxime |
| 88-B | 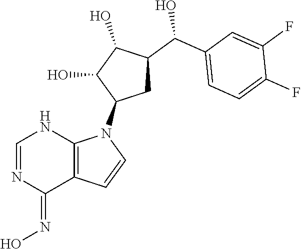 [SMILES: O/N=c1\nc[nH]c2c1ccn2[C@@H]1C[C@H]([C@H](O)c2ccc(F)c(F)c2)[C@@H](O)[C@H]1O] | 392.36 | (Z)-7-((1R,2S,3R,4R)-4-((S)-(3,4- difluorophenyl)(hydroxy)methyl)- 2,3-dihydroxycyclopentyl)-1,7- dihydro-4H-pyrrolo[2,3- d]pyrimidin-4-one oxime |
| 89-B | 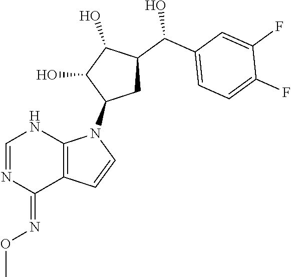 [SMILES: CO/N=c1\nc[nH]c2c1ccn2[C@@H]1C[C@H]([C@H](O)c2ccc(F)c(F)c2)[C@@H](O)[C@H]1O] | 406.38 | (Z)-7-((1R,2S,3R,4R)-4-((S)-(3,4- difluorophenyl)(hydroxy)methyl)- 2,3-dihydroxycyclopentyl)-1,7- dihydro-4H-pyrrolo[2,3- d]pyrimidin-4-one O-methyl oxime |
| 90-B | 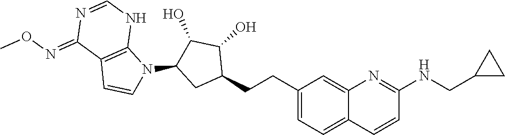 [SMILES: CO/N=c1\nc[nH]c2c1ccn2[C@@H]1C[C@H](CCc2ccc3ccc(NCC4CC4)nc3c2)[C@@H](O)[C@H]1O] | 488.58 | (Z)-7-((1R,2S,3R,4S)-4-(2-(2- ((cyclopropylmethyl)amino)quinolin- 7-yl)ethyl)-2,3- dihydroxycyclopentyl)-1,7-dihydro- 4H-pyrrolo[2,3-d]pyrimidin-4-one O-methyl oxime |
| 91-B | 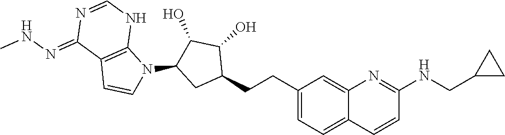 [SMILES: CN/N=c1\nc[nH]c2c1ccn2[C@@H]1C[C@H](CCc2ccc3ccc(NCC4CC4)nc3c2)[C@@H](O)[C@H]1O] | 487.6 | (1S,2R,3S,5R)-3-(2-(2- ((cyclopropylmethyl)amino)quinolin- 7-yl)ethyl)-5-((Z)-4-(2- methylhydrazineylidene)-1,4- dihydro-7H-pyrrolo[2,3- d]pyrimidin-7-yl)cyclopentane-1,2- diol |
| 92-B | 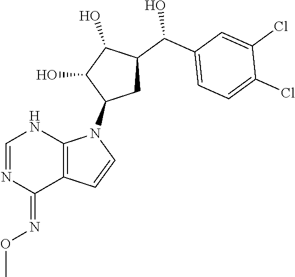 [SMILES: CO/N=c1\nc[nH]c2c1ccn2[C@@H]1C[C@H]([C@H](O)c2ccc(Cl)c(Cl)c2)[C@@H](O)[C@H]1O] | 439.29 | (Z)-7-((1R,2S,3R,4R)-4-((S)-(3,4- dichlorophenyl)(hydroxy)methyl)- 2,3-dihydroxycyclopentyl)-1,7- dihydro-4H-pyrrolo[2,3- d]pyrimidin-4-one O-methyl oxime |
| 93-B | 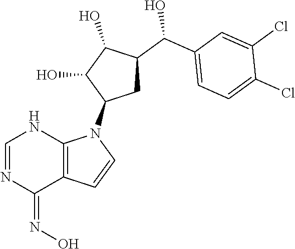 [SMILES: O/N=c1/nc[nH]c2c1ccn2[C@@H]1C[C@H]([C@H](O)c2ccc(Cl)c(Cl)c2)[C@@H](O)[C@H]1O] | 425.27 | (E)-7-((1R,2S,3R,4R)-4-((S)-(3,4- dichlorophenyl)(hydroxy)methyl)- 2,3-dihydroxycyclopentyl)-1,7- dihydro-4H-pyrrolo[2,3- d]pyrimidin-4-one oxime |
| 94-B | 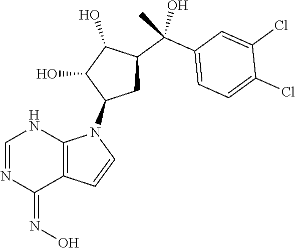 [SMILES: C[C@@](O)(c1ccc(Cl)c(Cl)c1)[C@H]1C[C@@H](n2ccc3/c(=N\O)nc[nH]c32)[C@H](O)[C@@H]1O] | 439.29 | (E)-7-((1R,2S,3R,4S)-4-((S)-1-(3,4- dichlorophenyl)-1-hydroxyethyl)- 2,3-dihydroxycyclopentyl)-1,7- dihydro-4H-pyrrolo[2,3- d]pyrimidin-4-one oxime |

Compounds of the disclosure of Formula V and Formula VI include, for example, the compounds identified in Table C.

TABLE C

| Ex. | | | |
|---|---|---|---|
| No. | Structure | MW | Chemical Name |
| 1-C | 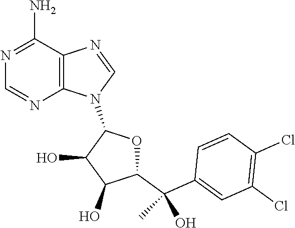 [SMILES: C[C@@](O)(c1ccc(Cl)c(Cl)c1)[C@H]1O[C@@H](n2cnc3c(N)ncnc32)[C@H](O)[C@@H]1O] | 426.254 | (2R,3R,4S,5S)-2-(6-amino-9H-purin-9-yl)- 5-((R)-1-(3,4-dichlorophenyl)-1- hydroxyethyl)tetrahydrofuran-3,4-diol |
| 2-C | 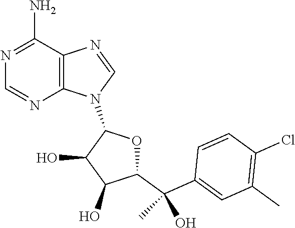 [SMILES: Cc1cc([C@@](C)(O)[C@H]2O[C@@H](n3cnc4c(N)ncnc43)[C@H](O)[C@@H]2O)ccc1Cl] | 405.839 | (2R,3R,4S,5S)-2-(6-amino-9H-purin-9-yl)- 5-((R)-1-(4-chloro-3-methylphenyl)-1- hydroxyethyl)tetrahydrofuran-3,4-diol |
| 3-C | 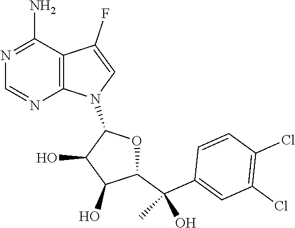 [SMILES: C[C@@](O)(c1ccc(Cl)c(Cl)c1)[C@H]1O[C@@H](n2cc(F)c3c(N)ncnc32)[C@H](O)[C@@H]1O] | 443.256 | (2R,3R,4S,5S)-2-(4-amino-5-fluoro-7H- pyrrolo[2,3-d]pyrimidin-7-yl)-5-((R)-1- (3,4-dichlorophenyl)-1- hydroxyethyl)tetrahydrofuran-3,4-diol |
| 4-C | 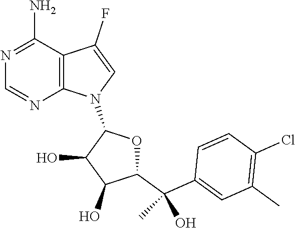 [SMILES: Cc1cc([C@@](C)(O)[C@H]2O[C@@H](n3cc(F)c4c(N)ncnc43)[C@H](O)[C@@H]2O)ccc1Cl] | 422.841 | (2R,3R,4S,5S)-2-(4-amino-5-fluoro-7H- pyrrolo[2,3-d]pyrimidin-7-yl)-5-((R)-1-(4- chloro-3-methylphenyl)-1- hydroxyethyl)tetrahydrofuran-3,4-diol |
| 5-C | 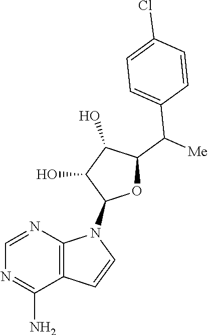 [SMILES: CC(c1ccc(Cl)cc1)[C@H]1O[C@@H](n2ccc3c(N)ncnc32)[C@H](O)[C@@H]1O] | 405.839 | (2R,3R,4S,5R)-2-(4-amino-7H- pyrrolo[2,3-d]pyrimidin-7-yl)-5-(1-(4- chlorophenyl)ethyl)tetrahydrofuran-3,4- diol |
| 6-C | 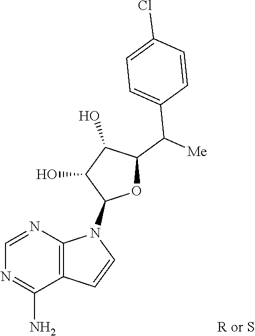 [SMILES: CC(c1ccc(Cl)cc1)[C@H]1O[C@@H](n2ccc3c(N)ncnc32)[C@H](O)[C@@H]1O] | 405.839 | (2R,3R,4S,5R)-2-(4-amino-7H- pyrrolo[2,3-d]pyrimidin-7-yl)-5-(1-(4- chlorophenyl)ethyl)tetrahydrofuran-3,4- diol |
| 7-C | 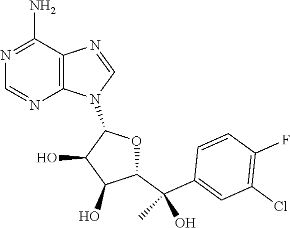 [SMILES: C[C@@](O)(c1ccc(F)c(Cl)c1)[C@H]1O[C@@H](n2cnc3c(N)ncnc32)[C@H](O)[C@@H]1O] | 4090802 | (2R,3R,4S,5S)-2-(6-amino-9H-purin-9-yl)- 5-((R)-1-(3-chloro-4-fluorophenyl)-1- hydroxyethyl)tetrahydrofuran-3,4-diol |
| 8-C | 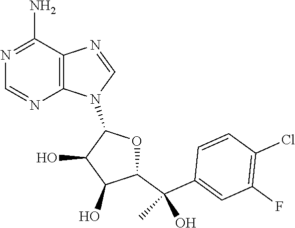 [SMILES: C[C@@](O)(c1ccc(Cl)c(F)c1)[C@H]1O[C@@H](n2cnc3c(N)ncnc32)[C@H](O)[C@@H]1O] | 409.802 | (2R,3R,4S,5S)-2-(6-amino-9H-purin-9-yl)- 5-((R)-1-(4-chloro-3-fluorophenyl)-1- hydroxyethyl)tetrahydrofuran-3,4-diol |
| 9-C | 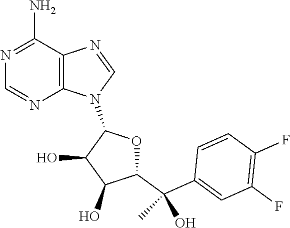 [SMILES: C[C@@](O)(c1ccc(F)c(F)c1)[C@H]1O[C@@H](n2cnc3c(N)ncnc32)[C@H](O)[C@@H]1O] | 393.351 | (2R,3R,4S,5S)-2-(6-amino-9H-purin-9-yl)- 5-((R)-1-(3,4-difluorophenyl)-1- hydroxyethyl)tetrahydrofuran-3,4-diol |
| 10-C | 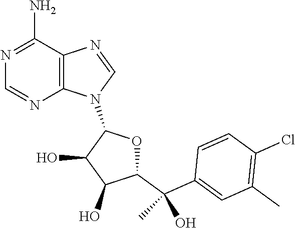 [SMILES: Cc1cc([C@@](C)(O)[C@H]2O[C@@H](n3cnc4c(N)ncnc43)[C@H](O)[C@@H]2O)ccc1Cl] | 405.839 | (2R,3R,4S,5S)-2-(6-amino-9H-purin-9-yl)- 5-((R)-1-(4-chloro-3-methylphenyl)-1- hydroxyethyl)tetrahydrofuran-3,4-diol |
| 11-C | 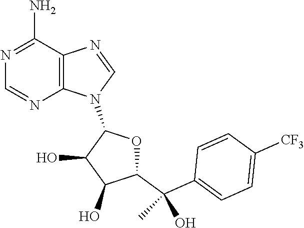 [SMILES: C[C@@](O)(c1ccc(C(F)(F)F)cc1)[C@H]1O[C@@H](n2cnc3c(N)ncnc32)[C@H](O)[C@@H]1O] | 425.368 | (2R,3R,4S,5S)-2-(6-amino-9H-purin-9-yl)- 5-((R)-1-hydroxy-1-(4- (trifluoromethyl)phenyl)ethyl)tetrahydro furan-3,4-diol |
| 12-C | 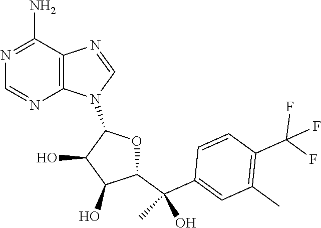 [SMILES: Cc1cc([C@@](C)(O)[C@H]2O[C@@H](n3cnc4c(N)ncnc43)[C@H](O)[C@@H]2O)ccc1C(F)(F)F] | 439.395 | (2R,3R,4S,5S)-2-(6-amino-9H-purin-9-yl)- 5-((R)-1-hydroxy-1-(3-methyl-4- (trifluoromethyl)phenyl)ethyl)tetrahydro furan-3,4-diol |
| 13-C | 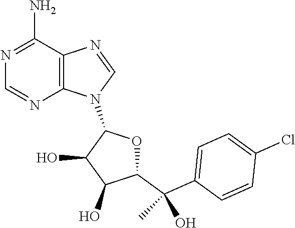 [SMILES: C[C@@](O)(c1ccc(Cl)cc1)[C@H]1O[C@@H](n2cnc3c(N)ncnc32)[C@H](O)[C@@H]1O] | 391.812 | (2R,3R,4S,5S)-2-(6-amino-9H-purin-9-yl)- 5-((R)-1-(4-chlorophenyl)-1- hydroxyethyl)tetrahydrofuran-3,4-diol |
| 14-C | 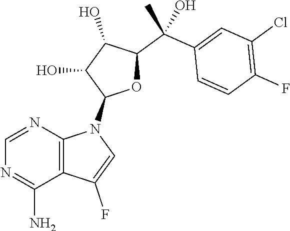 [SMILES: C[C@@](O)(c1ccc(F)c(Cl)c1)[C@H]1O[C@@H](n2cc(F)c3c(N)ncnc32)[C@H](O)[C@@H]1O] | 426.805 | (2R,3R,4S,5S)-2-(4-amino-5-fluoro-7H- pyrrolo[2,3-d]pyrimidin-7-yl)-5-((R)-1-(3- chloro-4-fluorophenyl)-1- hydroxyethyl)tetrahydrofuran-3,4-diol |
| 15-C | 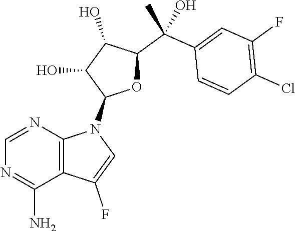 [SMILES: C[C@@](O)(c1ccc(Cl)c(F)c1)[C@H]1O[C@@H](n2cc(F)c3c(N)ncnc32)[C@H](O)[C@@H]1O] | 426.805 | (2R,3R,4S,5S)-2-(4-amino-5-fluoro-7H- pyrrolo[2,3-d]pyrimidin-7-yl)-5-((R)-1-(4- chloro-3-fluorophenyl)-1- hydroxyethyl)tetrahydrofuran-3,4-diol |
| 16-C | 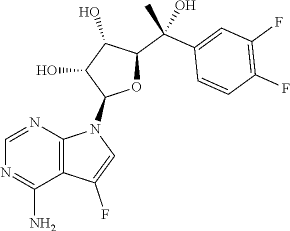 [SMILES: C[C@@](O)(c1ccc(F)c(F)c1)[C@H]1O[C@@H](n2cc(F)c3c(N)ncnc32)[C@H](O)[C@@H]1O] | 410.353 | (2R,3R,4S,5S)-2-(4-amino-5-fluoro-7H- pyrrolo[2,3-d]pyrimidin-7-yl)-5-((R)-1- (3,4-difluorophenyl)-1- hydroxyethyl)tetrahydrofuran-3,4-diol |
| 17-C | 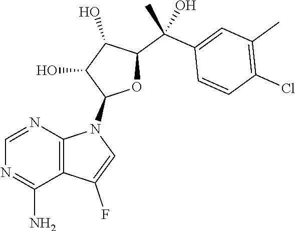 [SMILES: Cc1cc([C@@](C)(O)[C@H]2O[C@@H](n3cc(F)c4c(N)ncnc43)[C@H](O)[C@@H]2O)ccc1Cl] | 422.841 | (2R,3R,4S,5S)-2-(4-amino-5-fluoro-7H- pyrrolo[2,3-d]pyrimidin-7-yl)-5-((R)-1-(4- chloro-3-methylphenyl)-1- hydroxyethyl)tetrahydrofuran-3,4-diol |
| 18-C | 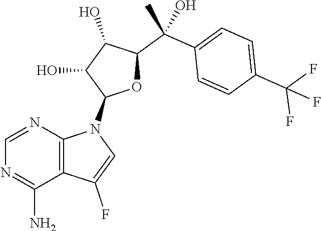 [SMILES: C[C@@](O)(c1ccc(C(F)(F)F)cc1)[C@H]1O[C@@H](n2cc(F)c3c(N)ncnc32)[C@H](O)[C@@H]1O] | 442.371 | (2R,3R,4S,5S)-2-(4-amino-5-fluoro-7H- pyrrolo[2,3-d]pyrimidin-7-yl)-5-((R)-1- hydroxy-1-(4- (trifluoromethyl)phenyl)ethyl)tetrahydro furan-3,4-diol |
| 19-C | 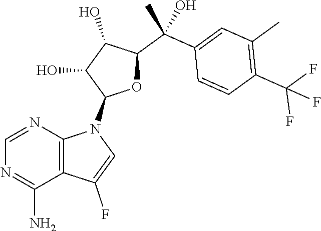 [SMILES: Cc1cc([C@@](C)(O)[C@H]2O[C@@H](n3cc(F)c4c(N)ncnc43)[C@H](O)[C@@H]2O)ccc1C(F)(F)F] | 456.398 | (2R,3R,4S,5S)-2-(4-amino-5-fluoro-7H- pyrrolo[2,3-d]pyrimidin-7-yl)-5-((R)-1- hydroxy-1-(3-methyl-4- (trifluoromethyl)phenyl)ethyl)tetrahydro furan-3,4-diol |
| 20-C | 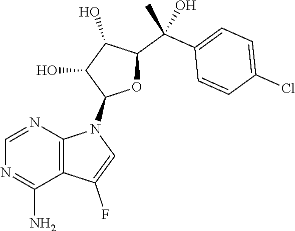 [SMILES: C[C@@](O)(c1ccc(Cl)cc1)[C@H]1O[C@@H](n2cc(F)c3c(N)ncnc32)[C@H](O)[C@@H]1O] | 408.814 | (2R,3R,4S,5S)-2-(4-amino-5-fluoro-7H- pyrrolo[2,3-d]pyrimidin-7-yl)-5-((R)-1-(4- chlorophenyl)-1- hydroxyethyl)tetrahydrofuran-3,4-diol |
| 21-C | 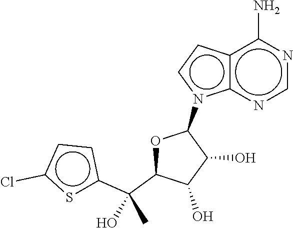 | 396.846 | (2R,3R,4S,5S)-2-(4-amino-7H- pyrrolo[2,3-d]pyrimidin-7-yl)-5-((S)-1-(5- chlorothiophen-2-yl)-1- hydroxyethyl)tetrahydrofuran-3,4-diol |
| 22-C | 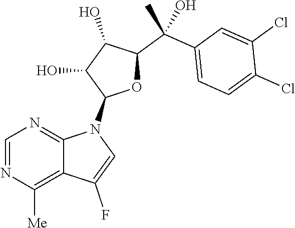 [SMILES: Cc1ncnc2c1c(F)cn2[C@@H]1O[C@H]([C@](C)(O)c2ccc(Cl)c(Cl)c2)[C@@H](O)[C@H]1O] | 442.268403 | (2S,3S,4R,5R)-2-((R)-1-(3,4- dichlorophenyl)-1-hydroxyethyl)-5-(5- fluoro-4-methyl-7H-pyrrolo[2,3- d]pyrimidin-7-yl)tetrahydrofuran-3,4-diol |
| 23-C | 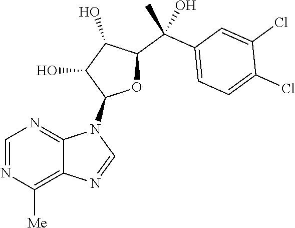 [SMILES: Cc1ncnc2c1ncn2[C@@H]1O[C@H]([C@](C)(O)c2ccc(Cl)c(Cl)c2)[C@@H](O)[C@H]1O] | 425.266 | (2S,3S,4R,5R)-2-((R)-1-(3,4- dichlorophenyl)-1-hydroxyethyl)-5-(6- methyl-9H-purin-9-yl)tetrahydrofuran- 3,4-diol |

### EXPERIMENTAL PROCEDURES

### Synthesis of [(R)-[(2S,3S,4R,5R)-5-(4-chloropyrrolo[2,3-d]pyrimidin-7-yl)-3,4-dihydroxy-tetrahydrofuran-2-yl]-[4-(trifluoromethyl)phenyl]methyl] 4-phenylbenzoate (Int-1)

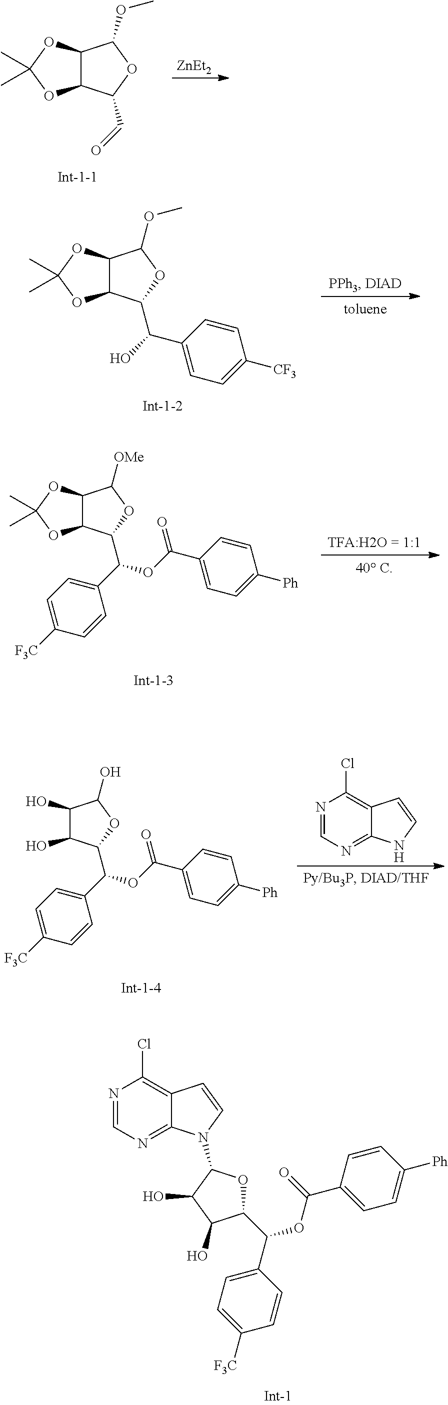

### Step 1. Preparation of (S)-[(3aR,4R,6R,6aR)-4-methoxy-2,2-dimethyl-3a,4,6,6a-tetrahydrofuro[3,4-d][1,3]dioxol-6-yl]-(4-chlorophenyl)methanol (Int-1-2)

To a solution of [4-(trifluoromethyl)phenyl]boronic acid (10 g, 52.65 mmol) in toluene (100 mL) was added diethylzine (157.95 mL, 157.95 mmol) at 25° C. The reaction mixture was stirred at 60° C. for 1 h. Then, (3aR,4R,6S,6aR)-4-methoxy-2,2-dimethyl-3a,4,6,6a-tetrahydrofuro[3,4-d][1,3]dioxole-6-carbaldehyde (Int-1-1) (10.65 g, 52.65 mmol) was added to the mixture and stirred at 60° C. for 3 hh. LCMS showed the reaction was completed and no Int-1-1 was left. The reaction was quenched with water and filtered. The mixture was concentrated in vacuum to give crude product, which was purified on a silica gel column (PE:EA=10:1 to 4:1) to afford (S)-[(3aR,4R,6R,6aR)-4-methoxy-2,2-dimethyl-3a,4,6,6a-tetrahydrofuro[3,4-d][1,3]dioxol-6-yl]-(4-chlorophenyl)methanol (Int-1-2) (4.7 g, 14.2 mmol, 26.9% yield) as white solid. ¹H NMR (400 MHz, CDCl₃) δ 7.63 (d, J=8.0 Hz, 2H), 7.49 (d, J=8.0 Hz, 2H), 4.96 (s, 1H), 4.93 (d, J=6.0 Hz, 1H), 4.77-4.80 (m, 1H), 4.71 (d, J=3.6 Hz, 1H), 4.68 (d, J=6.0 Hz, 1H), 4.12 (d, J=9.6 Hz, 1H), 3.37 (s, 3H), 1.47 (s, 3H), 1.33 (s, 3H).

### Step 2. Preparation of [(R)-[(3aR,4R,6R,6aR)-4-methoxy-2,2-dimethyl-3a,4,6,6a-tetrahydrofuro[3,4-d][1,3]dioxol-6-yl]-[4-(trifluoromethyl)phenyl]methyl] 4-phenylbenzoate (Int-1-3)

To a solution of (S)-[(3aR,4R,6R,6aR)-4-methoxy-2,2-dimethyl-3a,4,6,6a-tetrahydrofuro[3,4-d][1,3]dioxol-6-yl]-[4-(trifluoromethyl)phenyl]methanol (Int-1-2) (4.7 g, 12.9 mmol) in toluene (50 mL) was added triphenylphosphine (5 g, 19.4 mmol) and 4-biphenylcarboxylic acid (3.85 g, 19.4 mmol), then DIAD (3.83 mL, 19.4 mmol) was added at 0° C. The reaction mixture was stirred at room temperature for 3 h. TLC (PE:EA=5:1) showed the reaction was completed. The solvent was concentrated under reduced pressure. The residue was purified on a silica gel column (PE:EA=50:1 to PE:EA=30:1) to give [(R)-[(3aR,4R,6R,6aR)-4-methoxy-2,2-dimethyl-3a,4,6,6a-tetrahydrofuro[3,4-d][1,3]dioxol-6-yl]-[4-(trifluoromethyl)phenyl]methyl] 4-phenylbenzoate (Int-1-3) (5.7 g, 10.6 mmol, 81.6% yield) as a white solid. ¹H NMR (400 MHz, DMSO-d6) δ 8.13 (d, J=8.0 Hz, 2H), 7.86 (d, J=8.4 Hz, 2H), 7.74-7.78 (m, 6H), 7.52 (t, J=7.2 Hz, 2H), 7.44 (t, J=7.2 Hz, 1H), 5.97 (d, J=8.0 Hz, 1H), 5.03 (d, J=5.2 Hz, 1H), 4.94 (s, 1H), 4.69-4.72 (m, 2H), 3.15 (s, 3H), 1.40 (s, 3H), 1.27 (s, 3H).

### Step 3. Preparation of [(R)-[4-(trifluoromethyl)phenyl]-[(2S,3S,4R)-3,4,5-trihydroxytetrahydrofuran-2-yl]methyl] 4-phenylbenzoate (Int-1-4)

A solution of [(R)-[(3aR,4R,6R,6aR)-4-methoxy-2,2-dimethyl-3a,4,6,6a-tetrahydrofuro[3,4-d][1,3]dioxol-6-yl]-[4-(trifluoromethyl)phenyl]methyl] 4-phenylbenzoate (5.7 g, 10.6 mmol) in TFA (114 mL, 1.54 mol) and water (114 mL) was stirred at 40° C. for 18 hh. LCMS showed the reaction was completed. The solvent was concentrated under reduced pressure, and the residue was washed with NaHCO₃aqueous, extracted with EA. The combined organic layers were washed with brine, dried over anhydrous Na₂SO₄. The solvent was concentrated under reduced pressure and purified by silica gel column chromatography (PE:EA=10:1 to PE:EA=2:1) to give impure product (7.2 g) which was purified again by reversed-phase combi-flash eluting with H₂O:CH₃CN from 90:10 to 5:95 to give [(R)-[4-(trifluoromethyl)phenyl]-[(2S,3S,4R)-3,4,5-trihydroxytetrahydrofuran-2-yl]methyl] 4-phenylbenzoate (Int-1-4) (4.35 g, 8.99 mmol, 85.0% yield) as a white solid. ¹H NMR (400 MHz, DMSO-d6+D₂O) δ 8.16 (d, J=8.0 Hz, 2H), 7.86 (d, J=8.0 Hz, 2H), 7.69-7.77 (m, 6H), 7.53 (t, J=7.6 Hz, 2H), 7.45 (t, J=7.2 Hz, 1H), 6.05 (d, J=6.0 Hz, 1H), 4.99 (s, 1H), 4.22 (t, J=7.2 Hz, 1H), 4.15 (d, J=6.0 Hz, 1H), 3.70 (d, J=4.4 Hz, 1H), 4.06 (d, J=4.4 Hz, 1H), 2.76 (s, 3H).

### Step 4. Preparation of [(R)-[(2S,3S,4R,5R)-5-(4-chloropyrrolo[2,3-d]pyrimidin-7-yl)-3,4-dihydroxy-tetrahydrofuran-2-yl]-[4-(trifluoromethyl)phenyl]methyl] 4-phenylbenzoate (Int-1)

To a solution of 4-Chloro-7H-pyrrolo[2,3-d]pyrimidine (0.32 g, 2.1 mmol) and pyridine (0.17 mL, 2.1 mmol) in dry THE (10 mL) was added tributylphosphane (1.03 mL, 4.13 mmol) and DIAD (0.85 mL, 4.34 mmol) at 30° C. [(R)-[4-(trifluoromethyl)phenyl]-[(2S,3S,4R)-3,4,5-trihydroxytetrahydrofuran-2-yl]methyl] 4-phenylbenzoate (1 g, 2.1 mmol) in dry THE (10 mL) was added in one portion. The reaction mixture was stirred at 30° C. for 1 h. LCMS showed the reaction was completed. The crude product was purified by prep-HPLC (0.1% TFA) eluting with H₂O:CH₃CN from 90:10 to 5:95 to give [(R)-[(2S,3S,4R,5R)-5-(4-chloropyrrolo[2,3-d]pyrimidin-7-yl)-3,4-dihydroxy-tetrahydrofuran-2-yl]-[4-(trifluoromethyl)phenyl]methyl] 4-phenylbenzoate (Int-1) (330 mg, 0.53 mmol, 25.7% yield) as pale yellow solid. LCMS [M+H]: 610.3.

### Synthesis of (R)-((2S,3S,4R,5R)-5-(6-chloro-9H-purin-9-yl)-3,4-dihydroxytetrahydrofuran-2-yl)(4-chlorophenyl)methyl [1,1′-biphenyl]-4-carboxylate (Int-2)

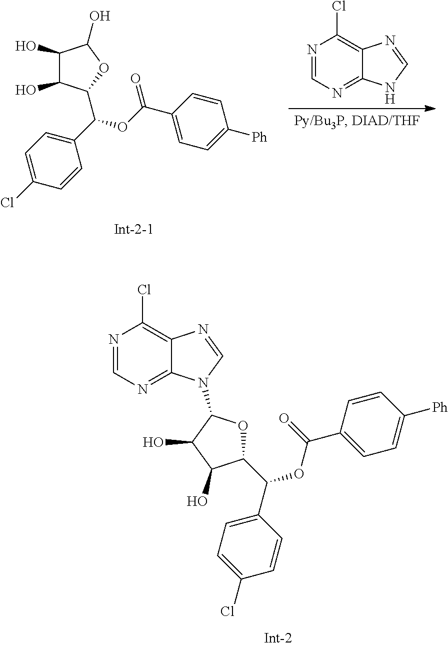

### Step 1. Preparation of (1R)-(4-chlorophenyl)((2S,3S,4R)-3,4,5-trihydroxytetrahydrofuran-2-yl)methyl[1,1′-biphenyl]-4-carboxylate (Int-2-1)

Int-2-1 was prepared similar to that of Int-1-4 except substituting [4-(trifluoromethyl)phenyl]boronic acid with (4-chlorophenyl)boronic acid.

### Step 2. Preparation of (R)-((2S,3S,4R,5R)-5-(6-chloro-9H-purin-9-yl)-3,4-dihydroxytetrahydrofuran-2-yl)(4-chlorophenyl)methyl [1,1′-biphenyl]-4-carboxylate (Int-2)

To a mixture of 6-Chloropurine (1.6 g, 10.6 mmol) in THE (40.0 mL) was added pyridine (0.8 mL, 10.6 mmol), tributyl phosphine (5.2 mL, 21.1 mmol) and DIAD (4.6 mL, 23.2 mmol). The mixture was cooled with ice-bath and [(R)-(4-chlorophenyl)-[(2S,3S,4R)-3,4,5-trihydroxy tetrahydrofuran-2-yl]methyl]4-phenylbenzoate (Int-2-1) (5.0 g, 10.6 mmol) in THE (30.0 mL) was added. The mixture was stirred at 25° C. for 2 h. The solvent was removed and the residue was purified by reversed phase combi-flash eluting with CH₃CN/H₂O (neutral condition) from 30/70 to 70/30 to give [(R)-(4-chlorophenyl)-[(2S,3S,4R,5R)-5-(6-chloropurin-9-yl)-3,4-dihydroxy-tetrahydrofuran-2-yl]methyl] 4-phenylbenzoate (2.6 g, 4.22 mmol, 40.0% yield) as yellow solid. LCMS: no MS signal. ¹H NMR (400 M Hz, DMSO-d6): δ 8.75 (s, 1H), 8.54 (s, 1H), 8.20 (d, J=4.4 Hz, 2H), 7.87 (d, J=4.4 Hz, 2H), 7.77 (d, J=4.0 Hz, 2H), 7.50-7.55 (m, 2H), 7.43-7.47 (m, 3H), 7.29 (d, J=4.0 Hz, 2H), 6.25 (d, J=5.2 Hz, 1H), 6.04 (d, J=5.6 Hz, 1H), 5.69 (d, J=6.0 Hz, 1H), 5.57 (d, J=5.2 Hz, 1H), 4.91-4.96 (m, 1H), 4.47-4.50 (m, 1H), 4.41-4.44 (m, 1H). ¹H NMR (400 M Hz, DMSO-d6+D₂O): δ 8.67 (s, 1H), 8.51 (s, 1H), 8.11 (d, J=4.4 Hz, 2H), 7.86 (d, J=8.4 Hz, 2H), 7.77 (d, J=7.6 Hz, 2H), 7.52-7.56 (m, 2H), 7.43-7.49 (m, 3H), 7.27 (d, J=8.4 Hz, 2H), 6.23 (d, J=4.4 Hz, 1H), 6.04 (d, J=5.2 Hz, 1H), 4.96 (t, J=4.8 Hz, 1H), 4.54 (t, J=4.8 Hz, 1H), 4.51 (t, J=4.8 Hz, 1H).

### Synthesis of (R)-(4-chloro-3-fluorophenyl)((3aR,4R,6R,6aR)-6-(4-chloro-7H-pyrrolo[2,3-d]pyrimidin-7-yl)-2,2-dimethyltetrahydrofuro[3[(R)-(4-chlorophenyl)-[(2R,3R,4R)-3,4,5-triacetoxytetra-hydrofuran-2-yl]methyl] 4-phenylbenzoate,4-d][1,3]dioxol-4-yl)methanol (Int-3)

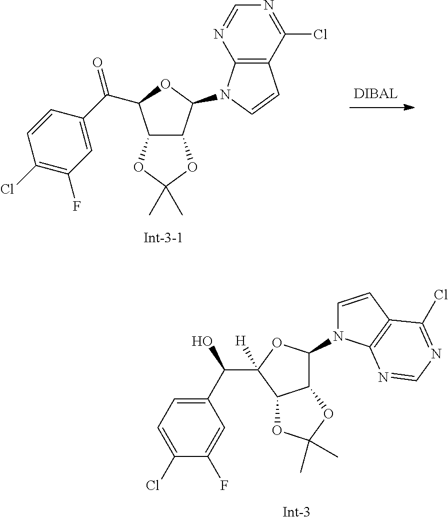 [SMILES: CC1(C)O[C@@H]2[C@H](O1)[C@@H](C(=O)c1ccc(Cl)c(F)c1)O[C@H]2n1ccc2c(Cl)ncnc21 ; CC1(C)O[C@H]2[C@@H](O1)[C@H](n1ccc3c(Cl)ncnc31)O[C@@H]2[C@H](O)c1ccc(Cl)c(F)c1]

Diisobutylalumanylium; hydride (DIBAL, 1.08 mL, 1.08 mmol) (1M in toluene) was added a solution of [(3aR,4R,6S,6aS)-4-(4-chloropyrrolo[2,3-d]pyrimidin-7-yl)-2,2-dimethyl-3a,4,6,6a-tetrahydrofuro[3,4-d][1,3]dioxol-6-yl]-(4-chloro-3-fluoro-phenyl)methanone (Int-3-1) (245 mg, 0.54 mmol) in Toluene (5 mL) at −78° C. The resulting mixture was stirred at −78° C. for 1 h. TLC showed small amount of remaining starting material, major desired product and small amount (more polar spot) wrong epimer. This was warmed up to room temperature, stirred for another 30 min, then cooled to 0° C. EtOAc was added, then the reaction mixture was poured into ice cold sat. aq. Rocher's salt. The aq. layer was extracted with EtOAc and the combined organic layers were washed with brine, dried over Na₂SO₄, filtered and concentrated. The residue was purified on a 20 g column which was eluted with 0-50% of EA/hexane to recover 20 mg starting ketone (8%); and to give (R)-[(3aR,4R,6R,6aR)-4-(4-chloropyrrolo[2,3-d]pyrimidin-7-yl)-2,2-dimethyl-3a,4,6,6a-tetrahydrofuro[3,4-d][1,3]dioxol-6-yl]-(4-chloro-3-fluoro-phenyl)methanol (Int-3) (163 mg, 0.359 mmol, 66.2% yield) as a white foamy solid.

### Synthesis of [(R)-(4-chlorophenyl)-[(2R,3R,4R,5R)-3,4-diacetoxy-5-(4-chloro-5-fluoro-pyrrolo[2,3-d]pyrimidin-7-yl)tetrahydrofuran-2-yl]methyl] 4-phenylbenzoate (Int-4)

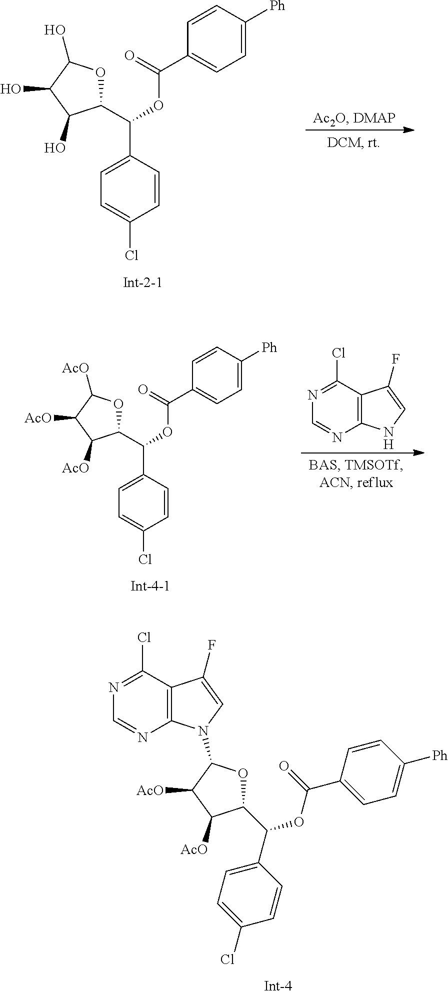

### Step 1. Preparation of [(R)-(4-chlorophenyl)-[(2R,3R,4R)-3,4,5-triacetoxytetra-hydrofuran-2-yl]methyl] 4-phenylbenzoate (Int-4-1)

To a solution of [(R)-(4-chlorophenyl)-[(2S,3S,4R)-3,4,5-trihydroxytetrahydrofuran-2-yl]methyl] 4-phenylbenzoate (Int-2-1) (4.0 g, 8.17 mmol) in pyridine (50 mL) was added DMAP (200.0 mg, 1.62 mmol), followed by acetic anhydride (3.86 mL, 40.7 mmol). The mixture was added at 23° C. for 16 h. After the reaction was complete, the solvent was removed under vacuum. The residue was purified by silica gel column chromatography (PE:EtOAc=4:1) to afford (3.6 g, 5.86 mmol, 71.8% yield) as white solid. LCMS [M+H]: 589.2.

### Step 2. Preparation of [(R)-(4-chlorophenyl)-[(2R,3R,4R,5R)-3,4-diacetoxy-5-(4-chloro-5-fluoro-pyrrolo[2,3-d]pyrimidin-7-yl)tetrahydrofuran-2-yl]methyl] 4-phenylbenzoate (Int-4)

To a solution of 4-chloro-5-fluoro-7H-pyrrolo[2,3-d]pyrimidine (600.0 mg, 3.46 mmol) in dry MeCN (30 mL) was added N,O-Bis(trimethylsilyl)acetamide (1 mL, 4.09 mmol). The mixture was stirred at 23° C. for 15 min. Then [(R)-(4-chlorophenyl)-[(2R,3R,4R)-3,4,5-triacetoxytetrahydrofuran-2-yl]methyl] 4-phenylbenzoate (Int-4-1) (2.15 g, 3.49 mmol) was added, followed by TMSOTf (1.0 mL, 5.53 mmol). The mixture was stirred at 82° C. for 16 h. After the reaction was complete, the solvent was removed in vacuo. The residue was purified by silica gel column chromatography (PE:EtOAc=3:1) to afford [(R)-(4-chlorophenyl)-[(2R,3R,4R,5R)-3,4-diacetoxy-5-(4-chloro-5-fluoro-pyrrolo[2,3-d]pyrimidin-7-yl)tetrahydrofuran-2-yl]methyl] 4-phenylbenzoate (Int-4) (300 mg, 0.385 mmol, 11% yield) as white solid. LCMS [M+H]: 680.3.

### Example 20. 7-[(2R,3R,4S,5R)-5-[(R)-(4-chlorophenyl)-hydroxy-methyl]-3,4-dihydroxy-tetrahydrofuran-2-yl]-1H-pyrrolo[2,3-d]pyrimidin-4-one hydrazone (20)

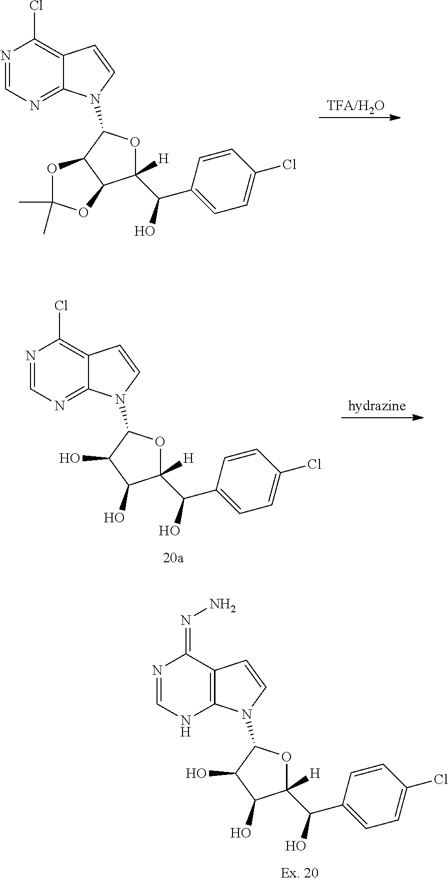 [SMILES: CC1(C)O[C@H]2[C@@H](O1)[C@H](n1ccc3c(Cl)ncnc31)O[C@@H]2[C@H](O)c1ccc(Cl)cc1 ; "TFA/H2O" ; O[C@@H]1[C@H](O)[C@@H]([C@H](O)c2ccc(Cl)cc2)O[C@H]1n1ccc2c(Cl)ncnc21 ; "hydrazine" ; N/N=c1/nc[nH]c2c1ccn2[C@@H]1O[C@H]([C@H](O)c2ccc(Cl)cc2)[C@@H](O)[C@H]1O]

### a) (2R,3S,4R,5R)-2-[(R)-(4-chlorophenyl)-hydroxy-methyl]-5-(4-chloropyrrolo[2,3-d]pyrimidin-7-yl)tetrahydrofuran-3,4-diol (20a)

A 50 mL RBF and septum containing (R)-[(3aR,4R,6R,6aR)-4-(4-chloropyrrolo[2,3-d]pyrimidin-7-yl)-2,2-dimethyl-3a,4,6,6a-tetrahydrofuro[3,4-d][1,3]dioxol-6-yl]-(4-chlorophenyl)methanol (Ref. PCT Int. Appl., 2016178870) (455 .mg, 1.04 mmol) was charged with a RT mixture of 2,2,2-trifluoroacetic acid (2.5 mL, 32.45 mmol) and Water (2.5 mL), sonicated for 10 s, blanketed with Ar, and stirred at RT for 2 h. The reaction mixture was concentrated under reduced pressure to remove the water and most of the TFA. The reaction was then diluted in MeOH (20 mL), and quenched with Amberlyst IRA-67 until a neutral pH was obtained. The mixture was then filtered through a cotton plug, rinsed with additional MeOH and DCM, and concentrated under reduced pressure to light brown foam. The crude product was purified by FCC (40 g SiO2, 3->4% MeOH in DCM, wet-loaded in DCM) to yield (2R,3S,4R,5R)-2-[(R)-(4-chlorophenyl)-hydroxy-methyl]-5-(4-chloropyrrolo[2,3-d]pyrimidin-7-yl)tetrahydrofuran-3,4-diol (20a) (128 mg, 0.31 mmol, 30.0% yield) as a white powder. Rf=0.26 (3% MeOH in DCM). LCMS (ESI) m/z calcd for [M+H]⁺ C₁₇H₁₆Cl₂N₃O₄: 396.051. Found: 396.0. ¹H NMR (400 MHz, DMSO-d₆) δ 8.67 (s, 1H), 8.02 (d, J=3.8 Hz, 1H), 7.44-7.32 (m, 4H), 6.79 (d, J=3.7 Hz, 1H), 6.19 (d, J=7.7 Hz, 1H), 6.02 (d, J=4.1 Hz, 1H), 5.39 (s, 1H), 5.21 (d, J=4.0 Hz, 1H), 4.80 (t, J=4.1 Hz, 1H), 4.58 (s, 1H), 4.12 (t, J=3.3 Hz, 1H), 4.00 (dd, J=5.3, 1.3 Hz, 1H). ¹H NMR of (400 MHz, DMSO-d6+D₂O) δ 8.66-8.60 (m, 1H), 7.96-7.87 (m, 1H), 7.42-7.29 (m, 4H), 6.76 (t, J=3.1 Hz, 1H), 6.15 (dd, J=7.6, 3.5 Hz, 1H), 4.76 (t, J=3.9 Hz, 1H), 4.55 (t, J=6.5 Hz, 1H), 4.11 (d, J=5.1 Hz, 1H), 4.01 (dd, J=5.1, 1.1 Hz, 1H).

### b) Synthesis of 7-[(2R,3R,4S,5R)-5-[(R)-(4-chlorophenyl)-hydroxy-methyl]-3,4-dihydroxy-tetrahydrofuran-2-yl]-1H-pyrrolo[2,3-d]pyrimidin-4-one hydrazine (20)

A 4 mL vial with septum containing (2R,3S,4R,5R)-2-[(R)-(4-chlorophenyl)-hydroxy-methyl]-5-(4-chloropyrrolo[2,3-d]pyrimidin-7-yl)tetrahydrofuran-3,4-diol (20a) (122 .mg, 0.3100 mmol, ˜9% 6-methoxy impurity) in IPA (0.6000 mL) was sparged with Ar for 1 min, then charged with hydrazine solution, 1 μM in THE (1.2 mL, 1.2 mmol). The vial was heated at 50° C. for 2 h, then stirred at rt for 16 h. The reaction mixture was dry loaded on Celite and purified by FCC (12 g SiO₂, 20->100% of 1:15:85 NH₄OH:MeOH:DCM in DCM). Desired fractions were combined, charged with 0.5 mL of 1 μM hydrazine in THF, stirred at RT for 5 min, and concentrated under reduced pressure to yield 7-[(2R,3R,4S,5R)-5-[(R)-(4-chlorophenyl)-hydroxy-methyl]-3,4-dihydroxy-tetrahydrofuran-2-yl]-1H-pyrrolo[2,3-d]pyrimidin-4-one hydrazone (20) (107.5 mg, 0.2661 mmol, 86.4% yield) as a white solid. Rf=0.2 (1:15:85 NH₄OH:MeOH:DCM). LCMS (ESI) m/z calcd for [M+H]⁺ C₁₇H₁₉ClN₅O₄: 392.112. Found: 392.1. ¹H NMR (400 MHz, DMSO-d₆) δ 8.75 (s, 1H), 8.13 (s, 1H), 7.47-7.34 (m, 4H), 7.32 (d, J=3.6 Hz, 1H), 6.69 (d, J=3.5 Hz, 2H), 5.92 (d, J=7.9 Hz, 1H), 5.23 (d, J=7.1 Hz, 1H), 5.04 (d, J=3.8 Hz, 1H), 4.81 (t, J=3.9 Hz, 1H), 4.62 (td, J=7.4, 4.8 Hz, 1H), 4.52 (s, 2H), 4.04-3.97 (m, 2H). ¹H NMR (400 MHz, DMSO-d₆+D₂O) δ 8.12 (s, 1H), 7.45-7.33 (m, 4H), 7.30 (d, J=3.6 Hz, 1H), 6.79-6.56 (m, 1H), 5.91 (d, J=7.9 Hz, 1H), 4.79 (d, J=4.3 Hz, 1H), 4.59 (dd, J=7.9, 5.0 Hz, 1H), 4.00 (d, J=4.5 Hz, 2H).

### Example 36 (2R,3S,4R,5R)-2-[(R)-(4-chlorophenyl)-hydroxy-methyl]-5-(-4-methoxyimino-1H-pyrrolo[2,3-d]pyrimidin-7-yl]tetrahydrofuran-3,4-diol (36)

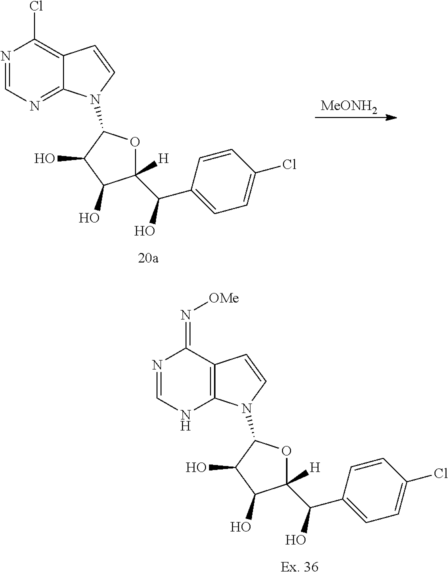 [SMILES: "MeONH2" ; O[C@@H]1[C@H](O)[C@@H]([C@H](O)c2ccc(Cl)cc2)O[C@H]1n1ccc2c(Cl)ncnc21 ; CO/N=c1/nc[nH]c2c1ccn2[C@@H]1O[C@H]([C@H](O)c2ccc(Cl)cc2)[C@@H](O)[C@H]1O]

A 4 mL vial containing a mixture of (R)-[(3aR,4R,6R,6aR)-2,2-dimethyl-4-(4-methylpyrrolo[2,3-d]pyrimidin-7-yl)-3a,4,6,6a-tetrahydrofuro[3,4-d][1,3]dioxol-6-yl]-(4-chlorophenyl)methanol (20a) (21 .mg, 0.050 mmol) and O-Methylhydroxylamine hydrochloride (22 .mg, 0.26 mmol) in 1,4-Dioxane (0.4 mL) and IPA (0.2 mL) was charged with Triethylamine (0.04 mL, 0.29 mmol). The vial was blanketed with Ar, sealed, and heated at 100° C. for 10 h. TLC showed consumption of SM. The mixture was concentrated under reduced pressure and purified by FCC (4 g SiO₂, 5->20% MeOH in DCM, wet-loaded in DCM+eluent). Fractions containing product were concentrated under reduced pressure and heat (50° C.) to yield (2R,3S,4R,5R)-2-[(R)-(4-chlorophenyl)-hydroxy-methyl]-5-(4-methoxyimino-1H-pyrrolo[2,3-d]pyrimidin-7-yl]tetrahydrofuran-3,4-diol (36) (20.4 mg, 0.0466 mmol, 88% yield) as a yellow/tan powder. Rf=0.4 (10% MeOH in DCM). LCMS (ESI) m/z calcd for [M+H]+C₁₈H₂₀ClN₄O₅: 407.112. Found: 407.0. ¹H NMR (400 MHz, DMSO-d₆) δ 10.71 (s, 1H), 8.11 (s, 1H), 7.66-7.52 (m, 1H), 7.44-7.33 (m, 4H), 6.68 (s, 1H), 6.03 (d, J=7.7 Hz, 1H), 4.77 (d, J=5.3 Hz, 1H), 4.50 (dd, J=7.8, 4.9 Hz, 1H), 4.08 (d, J=4.9 Hz, 1H), 3.97 (dd, J=5.3, 1.2 Hz, 1H), 3.82 (s, 3H), 3.76 (m, 2H), 3.07 (m, 1H).

### Example 37. 7-[(2R,3R,4S,5R)-5-[(R)-(4-chlorophenyl)-hydroxy-methyl]-3,4-dihydroxy-tetrahydrofuran-2-yl]-1H-pyrrolo[2,3-d]pyrimidin-4-one oxime hydrochloride (37)

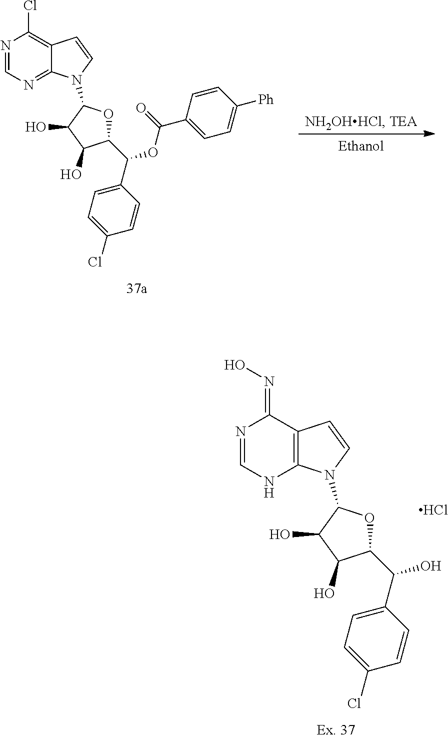 [SMILES: O=C(O[C@H](c1ccc(Cl)cc1)[C@H]1O[C@@H](n2ccc3c(Cl)ncnc32)[C@H](O)[C@@H]1O)c1ccc(-c2ccccc2)cc1 ; "NH2OH·HCl, TEA" ; O/N=c1\nc[nH]c2c1ccn2[C@@H]1O[C@H]([C@H](O)c2ccc(Cl)cc2)[C@@H](O)[C@H]1O ; Cl]

### a) Preparation of [(R)-(4-chlorophenyl)-[(2S,3S,4R,5R)-5-(4-chloropyrrolo[2,3-d]pyrimidin-7-yl)-3,4-dihydroxy-tetrahydrofuran-2-yl]methyl] 4-phenylbenzoate (37a)

Compound 37a was prepared similar to that of Int-1 except substituting [4-(trifluoromethyl)phenyl]boronic acid with (4-chlorophenyl)boronic acid.

### b) Preparation of 7-[(2R,3R,4S,5R)-5-[(R)-(4-chlorophenyl)-hydroxy-methyl]-3,4-dihydroxy-tetrahydrofuran-2-yl]-1H-pyrrolo[2,3-d]pyrimidin-4-one oxime hydrochloride (37)

To a solution of [(R)-(4-chlorophenyl)-[(2S,3S,4R,5R)-5-(4-chloropyrrolo[2,3-d]pyrimidin-7-yl)-3,4-dihydroxy-tetrahydrofuran-2-yl]methyl] 4-phenylbenzoate (500 mg, 0.65 mmol) in ethanol (10 mL) was added hydroxylamine hydrochloride (37a) (904.14 mg, 13.01 mmol) and TEA (2.26 mL, 16.26 mmol). The reaction mixture was heated to 90° C. and stirred for 5 hh. LCMS showed the reaction was completed. The solvent was concentrated under reduced pressure, and the residue was purified by prep-HPLC (0.1% TFA) eluting with H₂O:CH₃CN from 95:5 to 5:95 to give [(R)-(4-chlorophenyl)-[(2S,3S,4R,5R)-3,4-dihydroxy-5-(4-hydroxyimino-1H-pyrrolo[2,3-d]pyrimidin-7-yl]tetrahydrofuran-2-yl]methyl] 4-phenylbenzoate (146 mg, 0.25 mmol, 39.17% yield) and impure 7-[(2R,3R,4S,5R)-5-[(R)-(4-chlorophenyl)-hydroxy-methyl]-3,4-dihydroxy-tetrahydrofuran-2-yl]-1H-pyrrolo[2,3-d]pyrimidin-4-one oxime which was further purified by prep-TLC (DCM:MeOH=5:1). The product was dissolved in 1 M HCl, filtered and lyophilized to afford 7-[(2R,3R,4S,5R)-5-[(R)-(4-chlorophenyl)-hydroxy-methyl]-3,4-dihydroxy-tetrahydrofuran-2-yl]-1H-pyrrolo[2,3-d]pyrimidin-4-one oxime hydrochloride (37) (59.4 mg, 0.14 mmol, 20.88% yield) as pale yellow solid. LCMS [M+H]: 393.3. ¹H NMR (400 MHz, DMSO-d6) δ 8.28 (s, 1H), 7.75 (d, J=3.6 Hz, 1H), 7.35-7.42 (m, 4H), 7.01-7.03 (m, 1H), 6.11 (d, J=7.6 Hz, 1H), 4.77 (d, J=5.2 Hz, 1H), 4.49 (m, 1H), 4.13 (d, J=4.8 Hz, 1H), 3.99 (d, J=5.6 Hz, 1H). ¹H NMR (400 MHz, DMSO-d6+D₂O) δ 8.33 (s, 1H), 7.75 (d, J=3.6 Hz, 1H), 7.35-7.42 (m, 4H), 6.91 (d, J=3.6 Hz, 1H), 6.12 (d, J=7.2 Hz, 1H), 4.77 (d, J=5.2 Hz, 1H), 4.50 (m, 1H), 4.14 (d, J=4.8 Hz, 1H), 4.02 (d, J=5.2 Hz, 1H).

### Example 39. (2R,3S,4R,5R)-2-[(R)-(4-chlorophenyl)-hydroxy-methyl]-5-(6-hydrazinopurin-9-yl) tetrahydrofuran-3,4-diol; 2,2,2-trifluoroacetic acid (39)

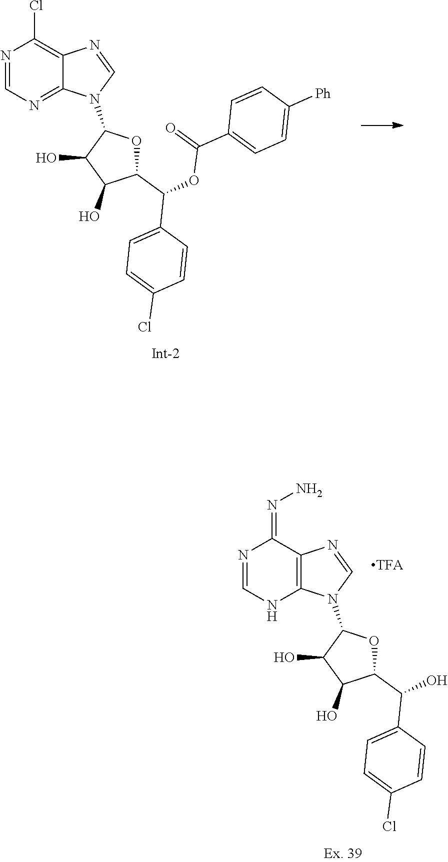 [SMILES: O=C(O[C@H](c1ccc(Cl)cc1)[C@H]1O[C@@H](n2cnc3c(Cl)ncnc32)[C@H](O)[C@@H]1O)c1ccc(-c2ccccc2)cc1 ; N/N=c1/nc[nH]c2c1ncn2[C@@H]1O[C@H]([C@H](O)c2ccc(Cl)cc2)[C@@H](O)[C@H]1O]

To a solution of [(R)-(4-chlorophenyl)-[(2S,3S,4R,5R)-5-(6-chloropurin-9-yl)-3,4-dihydroxy-tetrahydrofuran-2-yl]methyl] 4-phenylbenzoate (Int-2) (150 mg, 0.23 mmol) in ethanol (2 mL) was added hydrazine hydrate (2 mL) and stirred at 25° C. for 1 h. LCMS showed the reaction was completed. The solution was purified by prep-HPLC (0.1% TFA) eluting with H₂O:CH₃CN from 85:15 to 5:95 to give (2R,3S,4R,5R)-2-[(R)-(4-chlorophenyl)-hydroxy-methyl]-5-(6-hydrazinopurin-9-yl) tetrahydrofuran-3,4-diol; 2,2,2-trifluoroacetic acid (39) (43.1 mg, 0.083 mmol, 36.28% yield) as white solid. LCMS [M+H]: 393.3. ¹H NMR (400 MHz, DMSO-d6) δ 8.57 (s, 1H), 8.47 (s, 1H), 7.34-7.42 (m, 4H), 6.29 (s, 1H), 5.94 (d, J=7.6 Hz, 1H), 5.46 (s, 1H), 5.22 (s, 1H), 4.85 (d, J=5.2 Hz, 1H), 4.72-4.77 (m, 1H), 4.13 (d, J=5.2 Hz, 1H), 4.06 (d, J=4.4 Hz, 1H). ¹H NMR (400 MHz, DMSO-d6+D₂O) δ 8.56 (s, 1H), 8.48 (s, 1H), 7.35-7.42 (m, 4H), 5.95 (d, J=7.6 Hz, 1H), 4.85 (d, J=4.8 Hz, 1H), 4.72-4.77 (m, 1H), 4.14 (d, J=5.2 Hz, 1H), 4.09 (d, J=4.8 Hz, 1H).

### Example 52. (2R,3S,4R,5R)-2-[(R)-(4-chlorophenyl)-hydroxy-methyl]-5-[6-(methylhydrazono)-3H-purin-9-yl]tetrahydrofuran-3,4-diol; 2,2,2-trifluoroacetic acid (52)

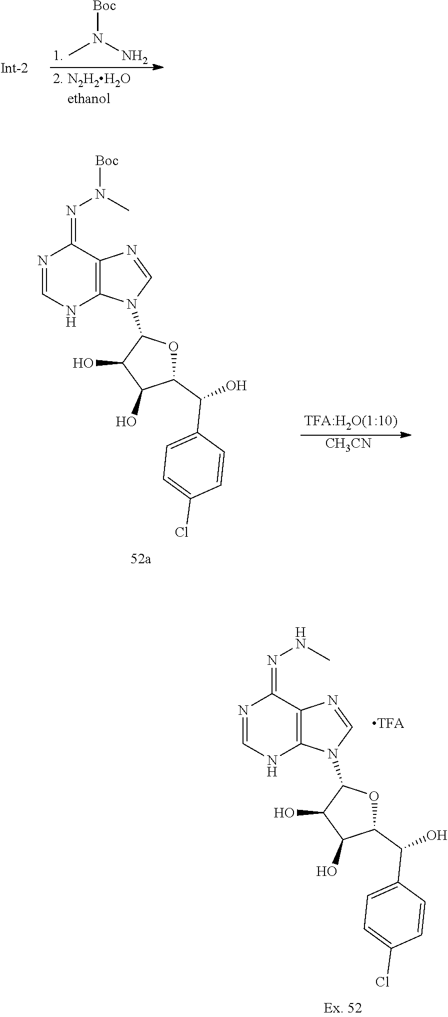

### a) Preparation of tert-butyl-2-(9-((2R,3R,4S,5R)-5-((R)-(4-chlorophenyl)(hydroxy)methyl)-3,4-dihydroxytetrahydrofuran-2-yl)-3H-purin-6(9H)-ylidene)-1-methylhydrazinecarboxylate (52a)

To a solution of (R)-((2S,3S,4R,5R)-5-(6-chloro-9H-purin-9-yl)-3,4-dihydroxytetrahydrofuran-2-yl) (4-chlorophenyl)methyl [1,1′-biphenyl]-4-carboxylate (Int-2) (375 mg, 0.65 mmol) in ethanol (2 mL) was added tert-butyl 1-methylhydrazinecarboxylate (2 mL). The reaction mixture was heated to 90° C. and stirred for 12 hh. LCMS showed the reaction was completed. The reaction mixture was cooled to rt. Hydrazine hydrate (2 mL) was added and stirred at 25° C. for 1.5 hh. LCMS showed the reaction was completed. The solvent was concentrated under reduced pressure to give crude product which was purified by silica gel column chromatography (DCM to DCM:CH₃CN=10:1 to DCM:CH₃OH=10:1) to give tert-butyl-2-(9-((2R,3R,4S,5R)-5-((R)-(4-chlorophenyl)(hydroxy)methyl)-3,4-dihydroxytetrahydrofuran-2-yl)-3H-purin-6(9H)-ylidene)-1-methylhydrazinecarboxylate (52a) (300 mg, 88% purity, 80% yield) as solid. LCMS [M+H]: 507.3.

### b) Preparation of (2R,3S,4R,5R)-2-[(R)-(4-chlorophenyl)-hydroxy-methyl]-5-[6-(methylhydrazono)-3H-purin-9-yl]tetrahydrofuran-3,4-diol; 2,2,2-trifluoroacetic acid (52)

To a solution of tert-butyl N-[[9-[(2R,3R,4S,5R)-5-[(R)-(4-chlorophenyl)-hydroxy-methyl]-3,4-dihydroxy-tetrahydrofuran-2-yl]-3H-purin-6-ylidene]amino]-N-methyl-carbamate (52a) (150 mg, 0.26 mmol) in MeCN (1 mL) and water (5 mL) was added 2,2,2-trifluoroacetic acid (0.5 mL). The reaction mixture was stirred at 25° C. for 18 h. LCMS showed the reaction was completed. The solution was purified by prep-HPLC (0.1% TFA) eluting with H₂O:CH₃CN from 85:15 to 5:95 to give (2R,3S,4R,5R)-2-[(R)-(4-chlorophenyl)-hydroxy-methyl]-5-[6-(methylhydrazono)-3H-purin-9-yl]tetrahydrofuran-3,4-diol; 2,2,2-trifluoroacetic acid (52) (30 mg, 0.0556 mmol, 21.36% yield) as white solid. LCMS [M+H]: 407.3. ¹H NMR (400 MHz, DMSO-d6) δ 8.61 (s, 1H), 8.45 (s, 1H), 7.34-7.42 (m, 4H), 6.22 (s, 1H), 5.94 (d, J=7.6 Hz, 1H), 5.47 (s, 1H), 5.24 (s, 1H), 4.84 (d, J=5.2 Hz, 1H), 4.71-4.73 (m, 1H), 4.14 (d, J=4.8 Hz, 1H), 4.06 (d, J=4.4 Hz, 1H), 2.76 (s, 3H). ¹H NMR (400 MHz, DMSO-d6+D₂O) δ 8.62 (s, 1H), 8.47 (s, 1H), 7.34-7.42 (m, 4H), 5.95 (d, J=7.6 Hz, 1H), 4.84 (d, J=5.2 Hz, 1H), 4.71-4.73 (m, 1H), 4.14 (d, J=5.2 Hz, 1H), 4.07 (d, J=5.2 Hz, 1H), 2.77 (s, 3H).

### Example 53. (2R,3S,4R,5R)-2-[(R)-(4-chlorophenyl)-hydroxy-methyl]-5-[6-methoxyimino-3H-purin-9-yl]tetrahydrofuran-3,4-diol trifluoroacetic acid salt (53)

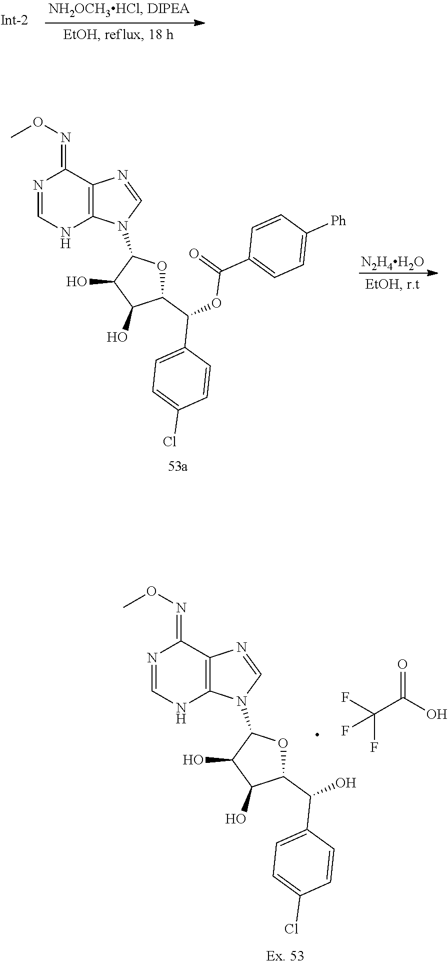

### a) Preparation of [(R)-(4-chlorophenyl)-[(2S,3S,4R,5R)-3,4-dihydroxy-5-[6-methoxyimino-3H-purin-9-yl]tetrahydrofuran-2-yl]methyl]-4-phenylbenzoate (53a)

To a solution of [(R)-(4-chlorophenyl)-[(2S,3S,4R,5R)-5-(6-chloropurin-9-yl)-3,4-dihydroxy-tetrahydrofuran-2-yl]methyl] 4-phenylbenzoate (Int-2) (100.0 mg, 0.16 mmol) in ethanol (4.0 mL), DIPEA (0.9 mL, 5.65 mmol) and methoxyammonium chloride (404.3 mg, 4.84 mmol) was added in portions. The mixture was stirred at 80° C. for 24 hh. LCMS showed the starting material was consumed completely. The mixture was used directly for the next step without further purification. LCMS [M+H]: 588.1.

### b) Preparation of (2R,3S,4R,5R)-2-[(R)-(4-chlorophenyl)-hydroxy-methyl]-5-[6-methoxyimino-3H-purin-9-yl]tetrahydrofuran-3,4-diol trifluoroacetic acid salt (53)

To the reaction mixture of last step (containing [(R)-(4-chlorophenyl)-[(2S,3 S,4R,5R)-3,4-dihydroxy-5-[6-methoxyimino-3H-purin-9-yl]tetrahydrofuran-2-yl]methyl]-4-phenylbenzoate (53a), hydrazine hydrate (3.0 mL, 61.73 mmol) was added. The mixture was stirred at 25° C. for 16 h. LCMS showed the starting material was consumed completely. The mixture was purified by prep-HPLC eluting with CH₃CN/H₂O (0.1% TFA contained) from 5/95 to 95/5 to give (2R,3S,4R,5R)-2-[(R)-(4-chlorophenyl)-hydroxy-methyl]-5-[6-methoxyimino-3H-purin-9-yl]tetrahydrofuran-3,4-diol (53, TFA salt, 22.0 mg, 0.04 mmol, 24.8% yield) as white solid. LCMS [M+H]: 408.1. ¹H NMR (400 M Hz, DMSO-d6): δ 8.19 (s, 1H), 7.80 (s, 1H), 7.36-7.42 (m, 4H), 5.78 (d, J=7.6 Hz, 1H), 4.81 (d, J=5.2 Hz, 1H), 4.62-4.65 (m, 1H), 4.08 (d, J=5.2 Hz, 1H), 4.01 (d, J=5.2 Hz, 1H), 3.77 (s, 3H). H NMR (400 M Hz, DMSO-d6+D₂O): δ 8.19 (s, 1H), 7.84 (s, 1H), 7.36-7.42 (m, 4H), 5.79 (d, J=7.6 Hz, 1H), 4.82 (d, J=4.8 Hz, 1H), 4.63 (m, 1H), 4.09 (d, J=5.2 Hz, 1H), 4.04 (d, J=5.2 Hz, 1H), 3.77 (s, 3H).

### Example 54. 9-[(2R,3R,4S,5R)-5-[(R)-(4-chlorophenyl)-hydroxy-methyl]-3,4-dihydroxy-tetrahydrofuran-2-yl]-3H-purin-6-one oxime trifluoroacetic acid salt (54)

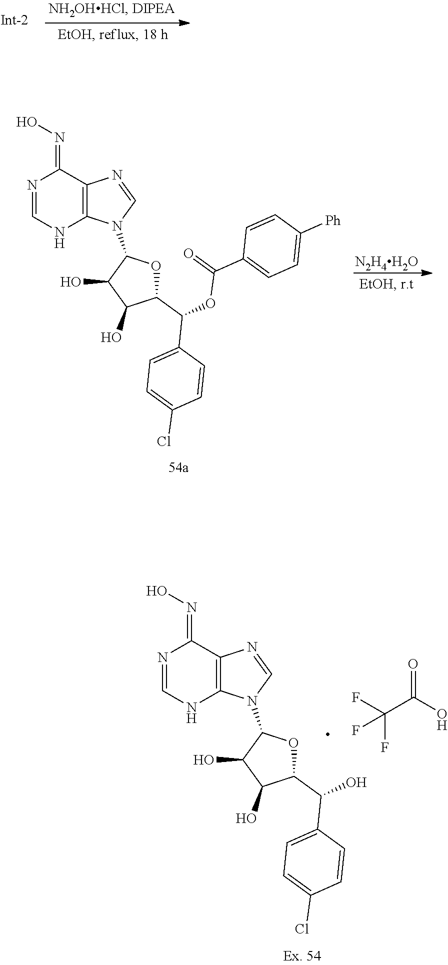

### a) Preparation of [(R)-(4-chlorophenyl)-[(2S,3S,4R,5R)-3,4-dihydroxy-5-[6-hydroxyimino-3H-purin-9-yl]tetrahydrofuran-2-yl]methyl]-4-phenylbenzoate (54a)

To a solution of [(R)-(4-chlorophenyl)-[(2S,3S,4R,5R)-5-(6-chloropurin-9-yl)-3,4-dihydroxy-tetrahydrofuran-2-yl]methyl] 4-phenylbenzoate (200.0 mg, 0.32 mmol) in ethanol (2.0 mL) was added DIPEA (0.4 mL, 2.58 mmol) and hydroxylamine hydrochloride (134.5 mg, 1.94 mmol). The mixture was stirred at 80° C. for 4 hh. LCMS showed the starting material was consumed completely and 30% desired product was detected in 254 nm. The mixture was used directly in the next step without further purification. LCMS [M+H]: 574.3.

### b) Preparation of 9-[(2R,3R,4S,5R)-5-[(R)-(4-chlorophenyl)-hydroxy-methyl]-3,4-dihydroxy-tetrahydrofuran-2-yl]-3H-purin-6-one oxime trifluoroacetic acid salt (54)

To the reaction mixture of last step (containing [(R)-(4-chlorophenyl)-[(2S,3 S,4R,5R)-3,4-dihydroxy-5-[6-hydroxyimino-3H-purin-9-yl]tetrahydrofuran-2-yl]methyl]-4-phenylbenzoate (54a), hydrazine hydrate (2.0 mL, 41.15 mmol) was added, the mixture was stirred at 25° C. for 16 h. LCMS showed the starting material was consumed completely. The mixture was purified by prep-HPLC eluting with CH₃CN/H₂O (0.1% TFA contained) from 5/95 to 95/5 to give 9-[(2R,3R,4S,5R)-5-[(R)-(4-chlorophenyl)-hydroxy-methyl]-3,4-dihydroxy-tetrahydrofuran-2-yl]-3H-purin-6-one oxime (54, TFA salt, 11.0 mg, 0.02 mmol, 22% yield) as white solid. LCMS [M+H]: 394.3. ¹H NMR (400 M Hz, DMSO-d6): δ 8.31 (s, 1H), 8.02 (s, 1H), 7.35-7.43 (m, 4H), 5.83 (d, J=7.6 Hz, 1H), 4.82 (d, J=4.8 Hz, 1H), 4.65-4.68 (m, 1H), 4.09 (d, J=4.8 Hz, 1H), 4.03 (d, J=4.8 Hz, 1H). ¹H NMR (400 M Hz, DMSO-d6+D₂O): δ 8.30 (s, 1H), 8.05 (s, 1H), 7.36-7.43 (m, 4H), 5.84 (d, J=7.6 Hz, 1H), 4.83 (d, J=4.8 Hz, 1H), 4.66 (m, 1H), 4.11 (d, J=4.8 Hz, 1H), 4.07 (d, J=4.8 Hz, 1H).

### Example 59. 7-((2R,3R,4S)-5-((R)-1-(4-chlorophenyl)-1-hydroxyethyl)-3,4-dihydroxytetrahydrofuran-2-yl)-1,7-dihydro-4H-pyrrolo[2,3-d]pyrimidin-4-one oxime hydrochloride (59)

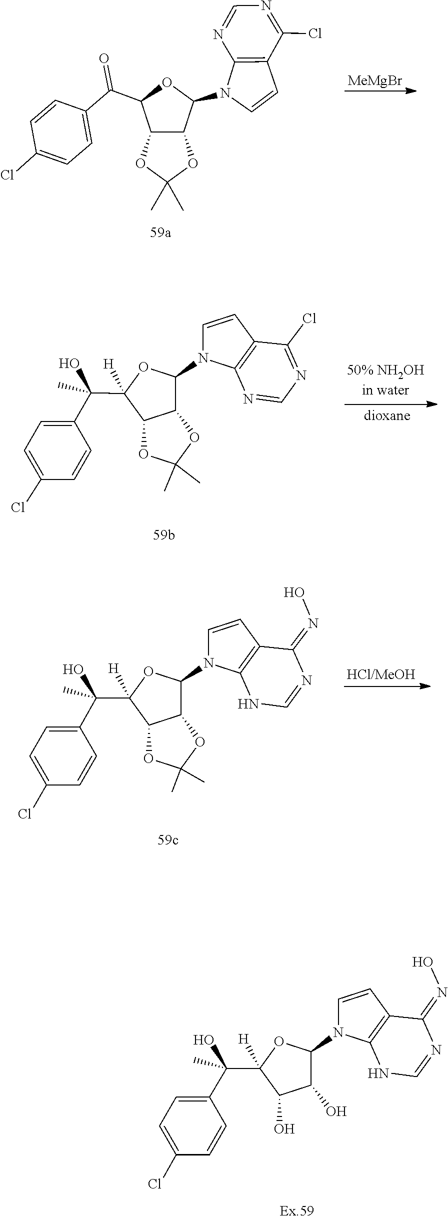 [SMILES: "MeMgBr" ; CC1(C)O[C@@H]2[C@H](O1)[C@@H](C(=O)c1ccc(Cl)cc1)O[C@H]2n1ccc2c(Cl)ncnc21 ; CC1(C)O[C@@H]2[C@H](O1)[C@@H]([C@](C)(O)c1ccc(Cl)cc1)O[C@H]2n1ccc2c(Cl)ncnc21 ; CC1(C)O[C@@H]2[C@H](O1)[C@@H]([C@](C)(O)c1ccc(Cl)cc1)O[C@H]2n1ccc2/c(=N\O)nc[nH]c21 ; C[C@@](O)(c1ccc(Cl)cc1)[C@H]1O[C@@H](n2ccc3/c(=N\O)nc[nH]c32)[C@H](O)[C@@H]1O]

### a) Preparation of (R)-1-((3aR,4S,6R,6aR)-6-(4-chloro-7H-pyrrolo[2,3-d]pyrimidin-7-yl)-2,2-dimethyltetrahydrofuro[3,4-d][1,3]dioxol-4-yl)-1-(4-chlorophenyl)ethan-1-ol (59b)

A mixture of compound 59a (300 mg, 691 umol, 1 eq.) in THE (3 mL) was degassed and purged with N₂ for 3 times, and then the mixture was cooled to 0° C. MeMgBr (3 M, 460.54 uL, 2 eq.) was added at 0° C., then the solution was stirred at 0° C. for 2 h under N₂ atmosphere. LC-MS showed compound 59a was consumed completely and one main peak with desired MS was detected. The reaction was quenched by sat. aq. NH₄Cl (5 mL), and extracted with EtOAc (5 mL*3), and the organic phase was concentrated in vacuo. The residue was purified by Prep-TLC (SiO₂, Petroleum ether:Ethyl acetate=5:1). Compound 59b (160 mg, crude) was obtained as a white solid. LCMS: (M+H⁺): 450.0; TLC (Petroleum ether:Ethyl acetate=5:1) R_{f}=0.28.

### b) Preparation of 7-((3aR,4R,6S,6aR)-6-((R)-1-(4-chlorophenyl)-1-hydroxyethyl)-2,2-dimethyltetrahydrofuro[3,4-d][1,3]dioxol-4-yl)-1,7-dihydro-4H-pyrrolo[2,3-d]pyrimidin-4-one oxime (59c)

To a solution of compound 59b (0.16 g, 355 umol, 1 eq.) in dioxane (5 mL) was added hydroxylamine (555 mg, 8.40 mmol, 0.5 mL, 23.6 eq.). The mixture was stirred at 120° C. for 12 h. LC-MS showed compound 59b was consumed completely and one main peak with desired MS was detected. The reaction was concentrated in vacuo. No purification. The crude product compound 59c (158 mg, crude) was used into the next step without further purification as a white solid. LCMS: (M−H⁺): 447.0;

### c) Preparation of 7-((2R,3R,4S,5S)-5-((R)-1-(4-chlorophenyl)-1-hydroxyethyl)-3,4-dihydroxytetrahydrofuran-2-yl)-1,7-dihydro-4H-pyrrolo[2,3-d]pyrimidin-4-one oxime (59)

A solution of compound 59c (158 mg, 354 umol, 1 eq.) in HCl/MeOH (4 M, 3 mL, 34 eq.) was stirred at 25° C. for 2 h. LC-MS showed compound 59c was consumed completely and one main peak with desired MS was detected. The mixture was concentrated in vacuo. The residue was purified by prep-HPLC (HCl condition). Compound 59 (130 mg, 292. umol, 83% yield, 99.65% purity, HCl salt) was obtained as a white solid. ¹H NMR (400 MHz, DMSO-d6) δ=8.29 (s, 1H), 7.77 (br d, J=3.1 Hz, 1H), 7.54 (d, J=8.8 Hz, 2H), 7.40 (d, J=8.8 Hz, 2H), 6.90 (br d, J=3.9 Hz, 1H), 6.10 (d, J=7.9 Hz, 1H), 4.37 (dd, J=5.0, 7.7 Hz, 1H), 4.06 (s, 1H), 3.71 (d, J=5.3 Hz, 1H), 1.42 (s, 3H); ¹H NMR (400 MHz, DMSO-d6+D₂O) δ=8.33 (s, 1H), 7.79 (d, J=3.5 Hz, 1H), 7.54 (d, J=8.8 Hz, 2H), 7.40 (d, J=8.3 Hz, 2H), 6.88 (d, J=3.5 Hz, 1H), 6.12 (d, J=7.9 Hz, 1H), 4.38 (dd, J=5.3, 7.9 Hz, 1H), 4.07 (s, 1H), 3.72 (d, J=4.8 Hz, 1H), 1.42 (s, 3H); LCMS: (M+H⁺): 407.0;

### Example 60. 7-((2R,3R,4S)-5-((R)-1-(4-chlorophenyl)-1-hydroxyethyl)-3,4-dihydroxytetrahydrofuran-2-yl)-1,7-dihydro-4H-pyrrolo[2,3-d]pyrimidin-4-one O-methyl oxime (60)

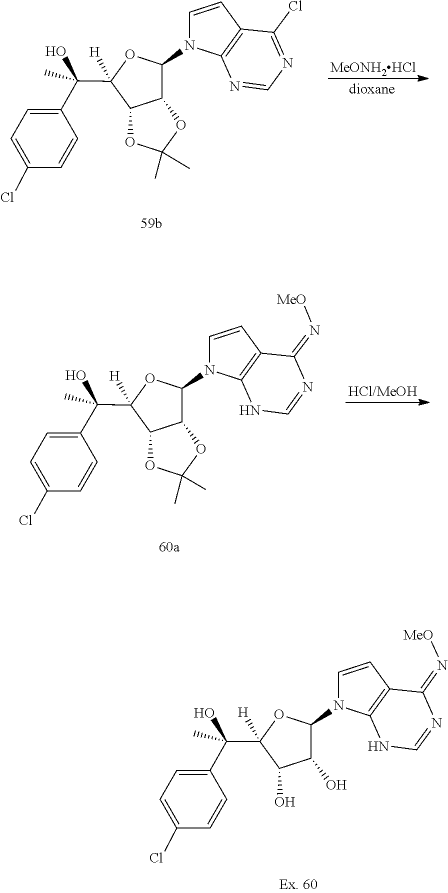 [SMILES: "MeONH2·HCl" ; CC1(C)O[C@@H]2[C@H](O1)[C@@H]([C@](C)(O)c1ccc(Cl)cc1)O[C@H]2n1ccc2c(Cl)ncnc21 ; CO/N=c1/nc[nH]c2c1ccn2[C@@H]1O[C@H]([C@](C)(O)c2ccc(Cl)cc2)[C@H]2OC(C)(C)O[C@H]21 ; CO/N=c1/nc[nH]c2c1ccn2[C@@H]1O[C@H]([C@](C)(O)c2ccc(Cl)cc2)[C@@H](O)[C@H]1O]

### a) Preparation of 7-((3aR,4R,6S,6aR)-6-((R)-1-(4-chlorophenyl)-1-hydroxyethyl)-2,2-dimethyltetrahydrofuro[3,4-d][1,3]dioxol-4-yl)-1,7-dihydro-4H-pyrrolo[2,3-d]pyrimidin-4-one O-methyl oxime (60a)

To a solution of compound 59b (30 mg, 66.32 umol, 1 eq.) in dioxane (6 mL) was added K₂CO₃ (274.99 mg, 1.99 mmol, 30 eq.) and O-methylhydroxylamine hydrochloride (110.78 mg, 1.33 mmol, 100.71 uL, 20 eq.). The mixture was stirred at 100° C. for 12 h in a sealed tube. LC-MS showed compound 59b was consumed completely and one main peak with desired MS was detected. The reaction was filtered and the filtrate was concentrated in vacuo. No purification. The crude product compound 60a (30 mg, crude) was used into the next step without further purification as a yellow oil. LCMS: (M+H⁺): 461.0;

### b) Preparation of 7-((2R,3R,4S)-5-((R)-1-(4-chlorophenyl)-1-hydroxyethyl)-3,4-dihydroxytetrahydrofuran-2-yl)-1,7-dihydro-4H-pyrrolo[2,3-d]pyrimidin-4-one O-methyl oxime (60)

To a solution of compound 60a (30 mg, 65 umol, 1 eq.) in MeOH (1 mL) was added HCl/MeOH (4 M, 1 mL, 61 eq.). The mixture was stirred at 25° C. for 1 h. LC-MS showed compound 60a was consumed completely and one main peak with desired MS was detected. The reaction was concentrated in vacuo at 25° C. The residue was purified by prep-HPLC. Ex. 60 (4.8 mg, 9.9 umol, 15% yield, 94.67% purity, HCl salt) was obtained as a white solid. ¹H NMR (400 MHz, DMSO-d6)=8.24 (s, 1H), 7.70 (br d, J=3.3 Hz, 1H), 7.55 (d, J=8.6 Hz, 2H), 7.40 (d, J=8.6 Hz, 2H), 6.82 (br d, J=2.9 Hz, 1H), 6.06 (d, J=7.7 Hz, 1H), 4.41 (dd, J=5.1, 7.7 Hz, 1H), 4.08 (s, 1H), 3.86 (s, 3H), 3.72 (br d, J=5.1 Hz, 1H); ¹H NMR (400 MHz, DMSO-d6+D₂O) δ=8.26 (s, 1H), 7.70 (d, J=3.7 Hz, 1H), 7.52 (d, J=8.6 Hz, 2H), 7.38 (d, J=8.6 Hz, 2H), 6.77 (d, J=3.5 Hz, 1H), 6.06 (d, J=7.9 Hz, 1H), 4.38 (dd, J=5.2, 7.8 Hz, 1H), 4.06 (s, 1H), 3.84 (s, 3H), 3.70 (d, J=5.3 Hz, 1H); LCMS: (M+H⁺): 421.1.

### Example 61. 7-((2R,3R,4S,5S)-5-((R)-1-(3,4-dichlorophenyl)-1-hydroxyethyl)-3,4-dihydroxytetrahydrofuran-2-yl)-1,7-dihydro-4H-pyrrolo[2,3-d]pyrimidin-4-one O-methyl oxime (61)

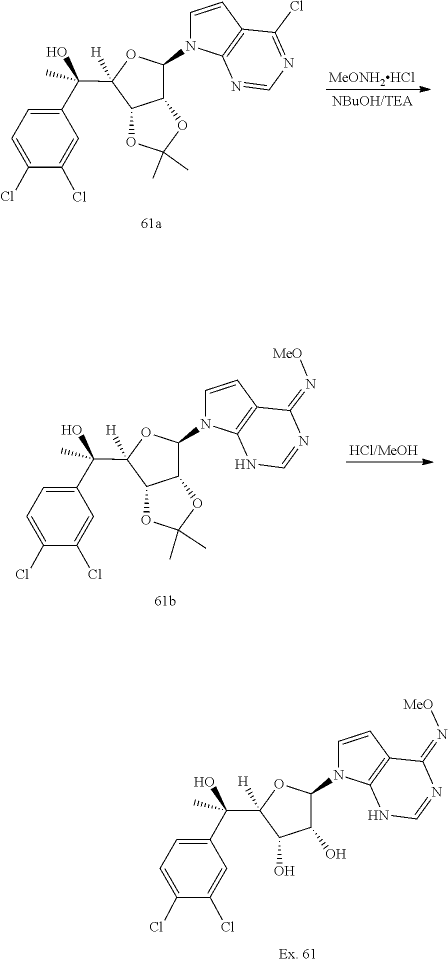 [SMILES: "MeONH2·HCl" ; CC1(C)O[C@@H]2[C@H](O1)[C@@H]([C@](C)(O)c1ccc(Cl)c(Cl)c1)O[C@H]2n1ccc2c(Cl)ncnc21 ; CO/N=c1/nc[nH]c2c1ccn2[C@@H]1O[C@H]([C@](C)(O)c2ccc(Cl)c(Cl)c2)[C@H]2OC(C)(C)O[C@H]21 ; CO/N=c1/nc[nH]c2c1ccn2[C@@H]1O[C@H]([C@](C)(O)c2ccc(Cl)c(Cl)c2)[C@@H](O)[C@H]1O]

### a) Preparation of ((1R)-1-[(3aR,4R,6S,6aR)-4-(4-chloropyrrolo[2,3-d]pyrimidin-7-yl)-2,2-dimethyl-3a,4,6,6a-tetrahydrofuro[3,4-d][1,3]dioxol-6-yl]-1-(3,4-dichlorophenyl)ethanol (61a)

Compound 61a was prepared following the procedure of (4-chloro-3-fluorophenyl)((3aS,4S,6R,6aR)-6-(4-chloro-7H-pyrrolo[2,3-d]pyrimidin-7-yl)-2,2-dimethyltetrahydrofuro[3,4-d][1,3]dioxol-4-yl)methanone (59b) except for substituting (4-chlorophenyl)((3aS,4S,6R,6aR)-6-(4-chloro-7H-pyrrolo[2,3-d]pyrimidin-7-yl)-2,2-dimethyltetrahydrofuro[3,4-d][1,3]dioxol-4-yl)methanone (59a) with (3,4-dichlorophenyl)((3aS,4S,6R,6aR)-6-(4-chloro-7H-pyrrolo[2,3-d]pyrimidin-7-yl)-2,2-dimethyltetrahydrofuro[3,4-d][1,3]dioxol-4-yl)methanone.

### b) Preparation of 7-((3aR,4R,6S,6aR)-6-((R)-1-(3,4-dichlorophenyl)-1-hydroxyethyl)-2,2-dimethyltetrahydrofuro[3,4-d][1,3]dioxol-4-yl)-1,7-dihydro-4H-pyrrolo[2,3-d]pyrimidin-4-one O-methyl oxime (61b)

A mixture of ((1R)-1-[(3aR,4R,6S,6aR)-4-(4-chloropyrrolo[2,3-d]pyrimidin-7-yl)-2,2-dimethyl-3a,4,6,6a-tetrahydrofuro[3,4-d][1,3]dioxol-6-yl]-1-(3,4-dichlorophenyl)ethanol (61a) (450 .mg, 0.7900 mmol), O-Methylhydroxylamine hydrochloride (347 mg, 3.95 mmol), and Triethylamine; TEA (0.67 mL, 4.73 mmol) in 1-Butanol (6 mL) was stirred in a sealed tube at 110° C. for 20 h. The reaction mixture was diluted with EtOAc and filtered. The filtrates were concentrated and purified on a 24 g column, which was eluted with 0-30% EA/DCM to give (1R)-1-[(3aR,4R,6S,6aR)-4-[(4E)-4-methoxyimino-1H-pyrrolo[2,3-d]pyrimidin-7-yl]-2,2-dimethyl-3a,4,6,6a-tetrahydrofuro[3,4-d][1,3]dioxol-6-yl]-1-(3,4-dichlorophenyl)ethanol (61b) (175 mg, 0.35 mmol, 44.8% yield).

### c) Preparation of 7-((2R,3R,4S,5S)-5-((R)-1-(3,4-dichlorophenyl)-1-hydroxyethyl)-3,4-dihydroxytetrahydrofuran-2-yl)-1,7-dihydro-4H-pyrrolo[2,3-d]pyrimidin-4-one O-methyl oxime (61)

To a solution of (1R)-1-[(3aR,4R,6S,6aR)-4-[(4E)-4-methoxyimino-1H-pyrrolo[2,3-d]pyrimidin-7-yl]-2,2-dimethyl-3a,4,6,6a-tetrahydrofuro[3,4-d][1,3]dioxol-6-yl]-1-(3,4-dichlorophenyl)ethanol (61b) (175 .mg, 0.35 mmol) in Methanol (5 mL) was added Hydrochloric acid (0.5 mL, 5.8 mmol) and stirred at room temperature for 3 h. The reaction was concentrated, the crude was treated with saturated NaHCO₃, extracted with ethyl acetate and the layers separated. The ethyl acetate layer was washed with water, brine, dried over sodium sulfate, filtered and concentrated. The crude was purified by silica gel chromatography on a 12 g Agela column using 0-30% EtOAc in DCM to give the product as free base, which was treated with 1M HCl and concentrated to give (2S,3S,4R,5R)-2-[(1R)-1-(3,4-dichlorophenyl)-1-hydroxy-ethyl]-5-[4-methoxyimino-1H-pyrrolo[2,3-d]pyrimidin-7-yl]tetrahydrofuran-3,4-diol hydrochloride (61) (130 mg, 0.264 mmol, 74.8% yield) as a light yellow solid. ¹H NMR (400 MHz, DMSO-d6+D₂O) δ 8.04 (s, 1H), 7.58-7.45 (m, 4H), 6.58 (d, J=3.6 Hz, 1H), 5.94 (d, J=7.2 Hz, 1H), 4.38 (m, 1H), 4.07 (s, 1H), 3.78 (s, 3H), 3.70 (d, J=5.2 Hz, 1H), 1.40 (S, 3H). LCMS [M+H]: 455.0/457.0.

### Example 62. 7-((2R,3R,4S,5R)-5-((R)-(4-chloro-3-fluorophenyl)(hydroxy)methyl)-3,4-dihydroxytetrahydrofuran-2-yl)-1,7-dihydro-4H-pyrrolo[2,3-d]pyrimidin-4-one O-methyl oxime (62)

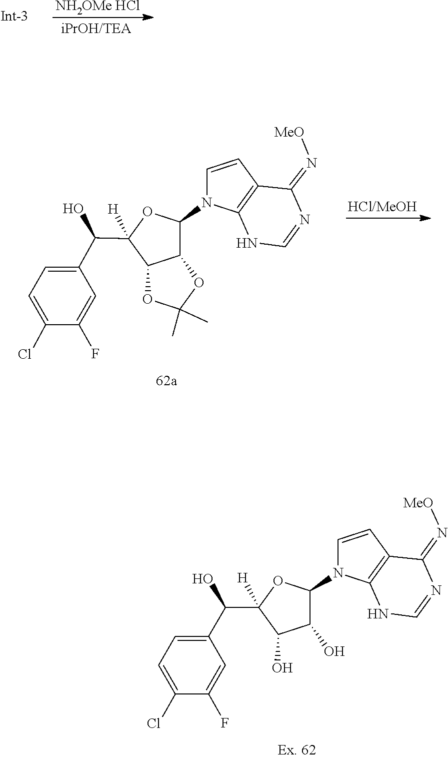 [SMILES: CO/N=c1/nc[nH]c2c1ccn2[C@@H]1O[C@H]([C@H](O)c2ccc(Cl)c(F)c2)[C@H]2OC(C)(C)O[C@H]21 ; "NH2OMe HCl" ; CO/N=c1/nc[nH]c2c1ccn2[C@@H]1O[C@H]([C@H](O)c2ccc(Cl)c(F)c2)[C@@H](O)[C@H]1O]

### a) Preparation of 7-((3aR,4R,6S,6aR)-6-((R)-1-(4-chloro-3-fluorophenyl)-1-hydroxyethyl)-2,2-dimethyltetrahydrofuro[3,4-d][1,3]dioxol-4-yl)-1,7-dihydro-4H-pyrrolo[2,3-d]pyrimidin-4-one O-methyl oxime (62a)

A mixture of (R)-[(3aR,4R,6R,6aR)-4-(4-chloropyrrolo[2,3-d]pyrimidin-7-yl)-2,2-dimethyl-3a,4,6,6a-tetrahydrofuro[3,4-d][1,3]dioxol-6-yl]-(4-chloro-3-fluoro-phenyl)methanol (Int-3) (70 .mg, 0.1500 mmol), O-Methylhydroxylamine hydrochloride (40.6 mg, 0.46 mmol) and Triethylamine; TEA (0.05 mL, 0.39 mmol) in IPA (1.5 mL) was heated at 110° C. Reaction was completed in 2 h by TLC (1:1 hexane/EA, product Rf˜0.3). The reaction mixture was diluted with EtOAc, filtered, concentrated, and the residue was purified on a 12 g column, eluted with 0-100 EA/hexane to give (R)-[(3aR,4R,6R,6aR)-4-[(4E)-4-methoxyimino-1H-pyrrolo[2,3-d]pyrimidin-7-yl]-2,2-dimethyl-3a,4,6,6a-tetrahydrofuro[3,4-d][1,3]dioxol-6-yl]-(4-chloro-3-fluoro-phenyl)methanol (62a) (37 mg, 0.080 mmol, 52% yield) as an off white solid.

### b) Preparation of 7-((2R,3R,4S,5R)-5-((R)-(4-chloro-3-fluorophenyl)(hydroxy)methyl)-3,4-dihydroxytetrahydrofuran-2-yl)-1,7-dihydro-4H-pyrrolo[2,3-d]pyrimidin-4-one O-methyl oxime (62)

A solution of (R)-[(3aR,4R,6R,6aR)-4-[4-(methoxyamino)pyrrolo[2,3-d]pyrimidin-7-yl]-2,2-dimethyl-3a,4,6,6a-tetrahydrofuro[3,4-d][1,3]dioxol-6-yl]-(4-chloro-3-fluoro-phenyl)methanol (62a) (37 .mg, 0.080 mmol) in pre-mixed TFA (0.9 mL, 12 mmol) and water (0.10 mL) was stirred at RT for 30 min. TLC showed most st.m. was consumed and a new spot was formed (10:1 DCM/MeOH, Rf˜0.4). The reaction mixture was concentrated and the residue was purified by C18 5.5 g column. The product fractions were combined, concentrated, and was re-dissolved in MeOH. A few drops of 1N HCl (aq.) was added and the mixture was concentrated to give 22 mg of compound 62 as an off-white solid. 19F NMR showed TFA was replaced. This was HCl salt. ¹H NMR (400 MHz, DMSO-d6+D₂O) δ 8.05 (br s, 1H), 7.58-7.45 (m, 4H), 6.60 (br s, 1H), 6.02 (d, J=8 Hz, 1H), 4.49 (m, 1H), 4.07 (d, J=4 Hz, 1H), 3.97 (m, 1H), 3.81 (s, 3H).

### Example 63. 7-((2R,3R,4S,5R)-5-((R)-(4-chloro-3-fluorophenyl)(hydroxy)methyl)-3,4-dihydroxytetrahydrofuran-2-yl)-1,7-dihydro-4H-pyrrolo[2,3-d]pyrimidin-4-one oxime (63)

Example 63 was prepared following the same procedures of Example 62 except for substituting NH₂OMe.HCl with NH₂OH.HCl. ¹H NMR (400 MHz, DMSO-d6+D₂O) δ 8.28 (s, 1H), 7.69 (br s, 1H), 7.49 (dd, J=8.8 Hz, 1H), 7.33 (d, J=8 Hz, 1H), 7.23 (m, 1H), 6.79 (d, J=4 Hz, 1H), 6.08 (d, J=8 Hz, 1H), 4.75 (d, J=4 Hz, 1H), 4.46 (dd, J=8, 4 Hz, 1H), 4.09 (m, 1H), 4.00 (m, 1H). LCMS [M+H]: 411.1/413.1.

### Example 64. (2R,3S,4R,5R)-2-[(R)-(4-chlorophenyl)-hydroxy-methyl]-5-[4-(methylhydrazono)-1H-pyrrolo[2,3-d]pyrimidin-7-yl]tetrahydrofuran-3,4-diol hydrochloride (64)

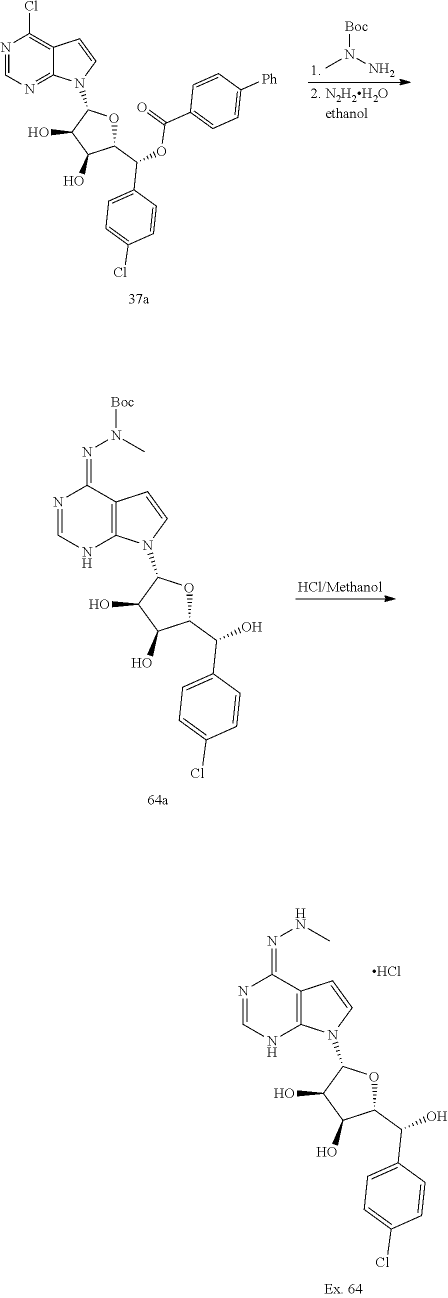

### a) Preparation of tert-butyl N-[7-[(2R,3R,4S,5R)-5-[(R)-(4-chlorophenyl)-hydroxy-methyl]-3,4-dihydroxy-tetrahydrofuran-2-yl]-1H-pyrrolo[2,3-d]pyrimidin-4-ylidene]amino-N-methyl-carbamate (64a)

To a solution of [(R)-(4-chlorophenyl)-[(2S,3S,4R,5R)-5-(4-chloropyrrolo[2,3-d]pyrimidin-7-yl)-3,4-dihydroxy-tetrahydrofuran-2-yl]methyl] 4-phenylbenzoate (37a) (300 mg, 0.39 mmol) in ethanol (2 mL) was added 1-Boc-1-methylhydrazine (2 mL, 13.48 mmol). The reaction mixture was heated to 90° C. and stirred for 18 hh. LCMS showed the reaction was complete. The reaction mixture was cold to 25° C., and hydrazine hydrate (2 mL) was added and stirred for 1 h. LCMS showed the reaction was completed. The solvent was concentrated under reduced pressure, the residue was washed with water, extracted with EA. The combined organic layers were washed with brine, dried over anhydrous Na₂SO₄, concentrated under reduced pressure to give crude tert-butyl N-[7-[(2R,3R,4S,5R)-5-[(R)-(4-chlorophenyl)-hydroxy-methyl]-3,4-dihydroxy-tetrahydrofuran-2-yl]-1H-pyrrolo[2,3-d]pyrimidin-4-ylidene]amino-N-methyl-carbamate (64a) (2 g, 0.23 mmol, 81.37% yield) which was used in the next step without further purification. LCMS [M+H]: 506.3.

### b) Preparation of (2R,3S,4R,5R)-2-[(R)-(4-chlorophenyl)-hydroxy-methyl]-5-[4-(methylhydrazono)-1H-pyrrolo[2,3-d]pyrimidin-7-yl]tetrahydrofuran-3,4-diol hydrochloride (64)

To a solution of tert-butyl N-[7-[(2R,3R,4S,5R)-5-[(R)-(4-chlorophenyl)-hydroxy-methyl]-3,4-dihydroxy-tetrahydrofuran-2-yl]-1H-pyrrolo[2,3-d]pyrimidin-4-ylidene]amino-N-methyl-carbamate (64a) (2 g, 0.24 mmol) in methanol (5 mL) was added HCl/MeOH (4 mol/L, 5 mL). The reaction mixture was heated to 90° C. and stirred for 2 h. LCMS showed the reaction was completed. The solvent was concentrated under reduced pressure, and the residue was purified by prep-HPLC (0.1% TFA) twice eluting with H₂O:CH₃CN from 95:5 to 5:95 to give (2R,3S,4R,5R)-2-[(R)-(4-chlorophenyl)-hydroxy-methyl]-5-[(4Z)-4-(methylhydrazono)-1H-pyrrolo[2,3-d]pyrimidin-7-yl]tetrahydrofuran-3,4-diol hydrochloride (64) (40 mg, 0.088 mmol, 37.02% yield) as white solid. LCMS [M+H]: 406.2. ¹H NMR (400 MHz, DMSO-d6) δ 10.90 (s, 1H), 8.28 (s, 1H), 7.72 (s, 1H), 7.35-7.42 (m, 4H), 6.88 (d, J=3.2 Hz, 1H), 6.10 (d, J=7.6 Hz, 1H), 6.00 (s, 1H), 5.31 (s, 1H), 5.15 (s, 1H), 4.77 (d, J=4.8 Hz, 1H), 4.49-4.52 (m, 1H), 4.11 (d, J=4.4 Hz, 1H), 4.00 (d, J=5.2 Hz, 1H), 2.67 (s, 3H). ¹H NMR (400 MHz, DMSO-d6+D₂O) δ 8.30 (s, 1H), 7.73 (d, J=3.2 Hz, 1H), 7.35-7.42 (m, 4H), 6.89 (d, J=3.6 Hz, 1H), 6.11 (d, J=7.6 Hz, 1H), 4.77 (d, J=5.2 Hz, 1H), 4.48-4.52 (m, 1H), 4.12 (d, J=4.8 Hz, 1H), 4.01 (d, J=4.4 Hz, 1H), 2.67 (s, 3H).

### Example 65. 7-((2R,3R,4S,5R)-5-((R)-(3-chloro-4-fluorophenyl)(hydroxy)methyl)-3,4-dihydroxytetrahydrofuran-2-yl)-1,7-dihydro-4H-pyrrolo[2,3-d]pyrimidin-4-one oxime (65)

Example 65 was prepared following similar procedures as those for Example 63 except for substituting [(3aR,4R,6S,6aS)-4-(4-chloropyrrolo[2,3-d]pyrimidin-7-yl)-2,2-dimethyl-3a,4,6,6a-tetrahydrofuro[3,4-d][1,3]dioxol-6-yl]-(4-chloro-3-fluoro-phenyl)methanone (Int-3-1) with [(3aR,4R,6S,6aS)-4-(4-chloropyrrolo[2,3-d]pyrimidin-7-yl)-2,2-dimethyl-3a,4,6,6a-tetrahydrofuro[3,4-d][1,3]dioxol-6-yl]-(3-chloro-4-fluoro-phenyl)methanone. ¹H NMR (400 MHz, DMSO-d₆) δ 8.30 (s, 1H), 7.77 (d, J=3.7 Hz, 1H), 7.55 (dd, J=7.3, 1.9 Hz, 1H), 7.44-7.31 (m, 2H), 6.89 (d, J=3.6 Hz, 1H), 6.10 (d, J=7.5 Hz, 1H), 4.78 (d, J=5.6 Hz, 1H), 4.48 (dd, J=7.6, 4.9 Hz, 1H), 4.12 (dd, J=4.9, 1.5 Hz, 1H), 3.98 (dd, J=5.7, 1.4 Hz, 1H).

### Example 66. 7-((2R,3R,4S,5R)-5-((R)-(3,4-dichlorophenyl)(hydroxy)methyl)-3,4-dihydroxytetrahydrofuran-2-yl)-1,7-dihydro-4H-pyrrolo[2,3-d]pyrimidin-4-one oxime (66)

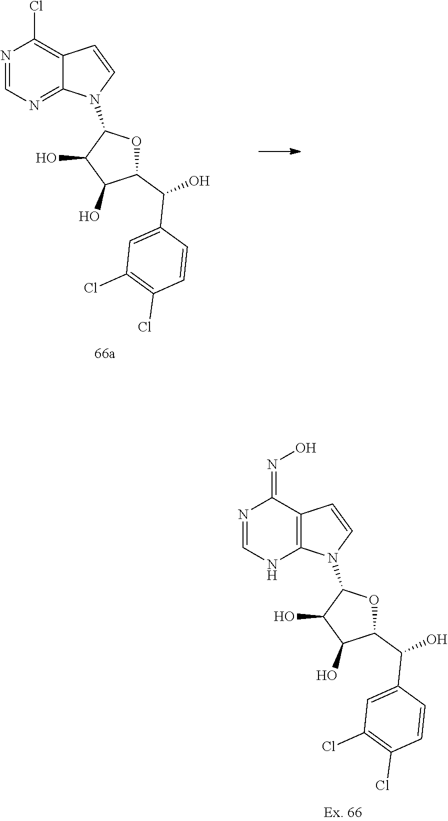 [SMILES: O[C@@H]1[C@H](O)[C@@H]([C@H](O)c2ccc(Cl)c(Cl)c2)O[C@H]1n1ccc2c(Cl)ncnc21 ; O/N=c1/nc[nH]c2c1ccn2[C@@H]1O[C@H]([C@H](O)c2ccc(Cl)c(Cl)c2)[C@@H](O)[C@H]1O]

### a) Preparation of (2R,3S,4R,5R)-2-[(R)-(3,4-dichlorophenyl)-hydroxy-methyl]-5-(4-chloropyrrolo[2,3-d]pyrimidin-7-yl)tetrahydrofuran-3,4-diol (66a)

Compound 66a was prepared following a similar procedure as that of compound 20a except substituting (R)-[(3aR,4R,6R,6aR)-4-(4-chloropyrrolo[2,3-d]pyrimidin-7-yl)-2,2-dimethyl-3a,4,6,6a-tetrahydrofuro[3,4-d][1,3]dioxol-6-yl]-(4-chlorophenyl)methanol with (R)-[(3aR,4R,6R,6aR)-4-(4-chloropyrrolo[2,3-d]pyrimidin-7-yl)-2,2-dimethyl-3a,4,6,6a-tetrahydrofuro[3,4-d][1,3]dioxol-6-yl]-(3,4-dichlorophenyl)methanol.

### b) Preparation of 7-((2R,3R,4S,5R)-5-((R)-(3,4-dichlorophenyl)(hydroxy)methyl)-3,4-dihydroxytetrahydrofuran-2-yl)-1,7-dihydro-4H-pyrrolo[2,3-d]pyrimidin-4-one oxime (66)

A 4 mL vial with septum containing (2R,3R,4S,5R)-2-(4-chloro-7H-pyrrolo[2,3-d]pyrimidin-7-yl)-5-((R)-(3,4-dichlorophenyl)(hydroxy)methyl)tetrahydrofuran-3,4-diol (66a) (56.5 mg, 0.130 mmol) and hydroxylamine; hydrochloride (45 mg, 0.65 mmol) was charged with IPA (2 mL) and then sparged with Ar for 1 min. The vial was then charged with Triethylamine; TEA (0.4 mL, 2.87 mmol) and heated at 100° C. for 5 h. The reaction mixture was concentrated under reduced pressure and purified by FCC (20 g C18, 5→35% MeCN in H₂O, wet-loaded in H₂O with TFA). Fractions containing product were combined and concentrated under reduced pressure. This material was further purified by FCC (30 g C18, 5→35% MeCN in H₂O, wet-loaded in H₂O+DMSO). Fractions containing only product by HPLC were combined, concentrated under reduced pressure, and repeatedly co-evaporated with 1 N HCl_{(aq)} and MeOH to remove TFA and yield the HCl salt of 7-((2R,3R,4S,5R)-5-((R)-(3,4-dichlorophenyl)(hydroxy)methyl)-3,4-dihydroxytetrahydrofuran-2-yl)-1,7-dihydro-4H-pyrrolo[2,3-d]pyrimidin-4-one oxime (66) (20. mg, 0.043 mmol, 33% yield) as a yellow solid. LRMS (ESI) m/z calcd for [M+H]⁺ C₁₇H₁₇C₁₂N₄O₅: 427.06/429.05. Found: 427.1/429.1; ¹H NMR (600 MHz, DMSO-d₆) δ 13.57 (s, 1H), 12.90 (s, 1H), 10.96 (s, 1H), 8.27 (s, 1H), 7.73 (s, 1H), 7.60 (d, J=1.9 Hz, 1H), 7.57 (d, J=8.3 Hz, 1H), 7.38 (dd, J=8.4, 2.0 Hz, 1H), 6.85 (d, J=3.6 Hz, 1H), 6.09 (d, J=7.5 Hz, 2H), 5.31 (s, 2H), 4.79 (d, J=5.5 Hz, 1H), 4.49 (dd, J=7.5, 5.0 Hz, 1H), 4.11 (dd, J=4.9, 1.6 Hz, 1H), 3.99 (dd, J=5.6, 1.6 Hz, 1H).

### Example 67. 7-((2R,3R,4S,5R)-5-((R)-(3,4-difluorophenyl)(hydroxy)methyl)-3,4-dihydroxytetrahydrofuran-2-yl)-1,7-dihydro-4H-pyrrolo[2,3-d]pyrimidin-4-one oxime (67)

Example 67 was prepared following similar procedures as those for Example 66 except for substituting (2R,3R,4S,5R)-2-(4-chloro-7H-pyrrolo[2,3-d]pyrimidin-7-yl)-5-((R)-(3,4-dichlorophenyl)(hydroxy)methyl)tetrahydrofuran-3,4-diol with (2R,3R,4S,5R)-2-(4-chloro-7H-pyrrolo[2,3-d]pyrimidin-7-yl)-5-((R)-(3,4-difluorophenyl)(hydroxy)methyl)tetrahydrofuran-3,4-diol. LRMS (ESI) m/z calcd for [M+H]⁺ C₁₇H₁₇F₂N₄O₅: 395.12. Found: 395.1; ¹H NMR (400 MHz, DMSO-d₆) δ 13.60 (s, 1H), 13.00 (s, 1H), 10.97 (s, 1H), 8.28 (s, 1H), 7.75 (d, J=3.9 Hz, 1H), 7.46-7.31 (m, 2H), 7.27-7.21 (m, 1H), 6.85 (d, J=3.6 Hz, 1H), 6.20-5.92 (m, 2H), 5.35 (s, 2H), 4.77 (d, J=5.5 Hz, 1H), 4.48 (dd, J=7.7, 5.0 Hz, 1H), 4.11 (dd, J=4.9, 1.4 Hz, 1H), 3.99 (dd, J=5.5, 1.4 Hz, 1H).

### Example 68. 7-((2R,3R,4S,5R)-5-((R)-(3-chloro-4-fluorophenyl)(hydroxy)methyl)-3,4-dihydroxytetrahydrofuran-2-yl)-1,7-dihydro-4H-pyrrolo[2,3-d]pyrimidin-4-one O-methyl oxime (68)

Example 68 was prepared following similar procedures as those for Example 62 except for substituting Int-3 with (R)-[(3aR,4R,6R,6aR)-4-(4-chloropyrrolo[2,3-d]pyrimidin-7-yl)-2,2-dimethyl-3a,4,6,6a-tetrahydrofuro[3,4-d][1,3]dioxol-6-yl]-(3-chloro-4-fluorophenyl)methanol. ¹H NMR (400 MHz, Methanol-d4) δ 8.15 (s, 1H), 7.62-7.48 (m, 2H), 7.43-7.29 (m, 1H), 7.20 (t, J=8.8 Hz, 1H), 6.67 (d, J=3.7 Hz, 1H), 6.15 (d, J=6.8 Hz, 1H), 4.60 (dd, J=4.9, 6.8 Hz, 1H), 4.29-4.19 (m, 2H), 3.94 (s, 3H).

### Example 69. 7-((2R,3R,4S,5R)-5-((R)-(3,4-dichlorophenyl)(hydroxy)methyl)-3,4-dihydroxytetrahydrofuran-2-yl)-1,7-dihydro-4H-pyrrolo[2,3-d]pyrimidin-4-one O-methyl oxime hydrochloride (69)

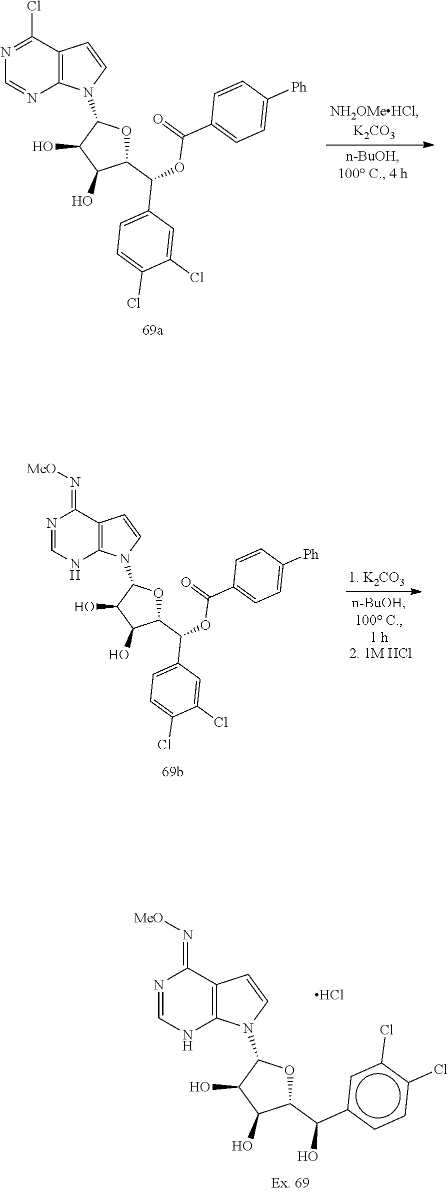

### a) Preparation of [(R)-[(2S,3S,4R,5R)-5-(4-chloropyrrolo[2,3-d]pyrimidin-7-yl)-3,4-dihydroxy-tetrahydrofuran-2-yl]-(3,4-dichlorophenyl)methyl] 4-phenylbenzoate (69a)

Compound 69a was prepared similar to that of Int-1 except substituting [4-(trifluoromethyl)phenyl]boronic acid with (3,4-dichlorophenyl)boronic acid.

### b) Preparation of [(R)-(3,4-dichlorophenyl)-[(2S,3S,4R,5R)-3,4-dihydroxy-5-[4-methoxyimino-1H-pyrrolo[2,3-d]pyrimidin-7-yl]tetrahydrofuran-2-yl]methyl] 4-phenylbenzoate (69b)

To a solution of [(R)-[(2S,3S,4R,5R)-5-(4-chloropyrrolo[2,3-d]pyrimidin-7-yl)-3,4-dihydroxy-tetrahydrofuran-2-yl]-(3,4-dichlorophenyl)methyl] 4-phenylbenzoate (69a) (10 g, 13.1 mmol) in 1-butanol (200 mL) was added methoxyammonium chloride (7.66 g, 91.7 mmol) and K₂CO₃ (18.07 g, 131 mmol). The reaction mixture was stirred at 100° C. for 4 hh. LCMS showed the reaction was completed. The solvent was concentrated under reduced pressure, and the residue was washed with water, extracted with EtOAc, dried over anhydrous Na₂SO₄. The solvent was removed in vacuum to give crude [(R)-(3,4-dichlorophenyl)-[(2S,3S,4R,5R)-3,4-dihydroxy-5-[4-methoxyimino-1H-pyrrolo[2,3-d]pyrimidin-7-yl]tetrahydrofuran-2-yl]methyl] 4-phenylbenzoate (10 g, 8.05 mmol, 61.4% yield) as oil (69b). LCMS [M+H]: 621.3.

### c) Preparation of (2R,3S,4R,5R)-2-[(R)-(3,4-dichlorophenyl)-hydroxy-methyl]-5-[(4Z)-4-methoxyimino-1H-pyrrolo[2,3-d]pyrimidin-7-yl]tetrahydrofuran-3,4-diol hydrochloride (69)

To a solution of [(R)-(3,4-dichlorophenyl)-[(2S,3S,4R,5R)-3,4-dihydroxy-5-[4-methoxyimino-1H-pyrrolo[2,3-d]pyrimidin-7-yl]tetrahydrofuran-2-yl]methyl] 4-phenylbenzoate (69a) (10 g, 8.05 mmol) in 1-butanol (100 mL) was added potassium carbonate (1.67 g, 12.1 mmol). The reaction mixture was stirred at 100° C. for 1.5 h. LCMS showed the reaction was completed. HCl aqueous (1M) was added to adjust pH=7.0, and the solvent was concentrated under reduced pressure. The residue was dissolved in DMSO, filtered. The filtrate was purified by reversed-phase combi-flash to give the impure product (5.5 g). The impure product was further purified by recrystallization (the material was suspended in the hot methanol and then filtered after the mixture was cooled to room temperature) from CH₃OH to afford pure (2R,3S,4R,5R)-2-[(R)-(3,4-dichlorophenyl)-hydroxy-methyl]-5-[4-methoxyimino-1H-pyrrolo[2,3-d]pyrimidin-7-yl]tetrahydrofuran-3,4-diol (2.4 g, 5.40 mmol, 67% yield, free base) as a pale yellow solid. Mp. 131.3° C. The mother liquid was purified by prep-HPLC (0.1% TFA) eluting with H₂O:CH₃CN from 90:10 to 5:95 to give crude TFA salt which was dissolved in 1M HCl and lyophilized to give (2R,3 S,4R,5R)-2-[(R)-(3,4-dichlorophenyl)-hydroxy-methyl]-5-[4-methoxyimino-1H-pyrrolo[2,3-d]pyrimidin-7-yl]tetrahydrofuran-3,4-diol hydrochloride (69) (1.1 g, 2.19 mmol, 27.2% yield). Mp. 131.3° C.; LCMS [M+H]: 441.3. ¹H NMR (400 MHz, DMSO-d6) δ 10.95 (d, J=3.2 Hz, 1H), 10.64 (s, 0.5H), 8.17 (s, 0.5H), 7.56-7.62 (m, 4H), 7.47 (d, J=4.0 Hz, 1H), 7.36-7.40 (m, 2H), 7.18 (d, J=3.6 Hz, 1H), 6.59 (d, J=3.6 Hz, 0.6H), 6.25 (d, J=3.2 Hz, 1H), 6.14 (d, J=4.4 Hz, 1H), 6.02 (d, J=7.2 Hz, 0.6H), 5.86 (d, J=7.6 Hz, 1H), 5.21-5.24 (m, 1.6H), 5.08 (d, J=4.4 Hz, 0.6H), 5.05 (d, J=4.0 Hz, 1H), 4.81 (t, J=4.8 Hz, 0.7H), 4.76 (t, J=4.8 Hz, 1H), 4.56 (b, 0.7H), 4.40-4.45 (m, 1H), 4.02-4.06 (m, 1.8H), 3.99 (d, J=4.8 Hz, 0.7H), 3.93 (d, J=4.8 Hz, 1H), 3.75 (s, 2H), 3.72 (s, 3H). ¹H NMR (400 MHz, DMSO-d6+D₂O) δ 8.20 (b, 1H), 7.56-7.62 (m, 3.7H), 7.49 (s, 1H), 7.36-7.40 (m, 1.8H), 7.17 (d, J=3.2 Hz, 1H), 6.59 (d, J=3.6 Hz, 1H), 6.26 (d, J=3.2 Hz, 1H), 6.04 (b, 0.6H), 5.86 (d, J=7.6 Hz, 1H), 4.81 (d, J=4.8 Hz, 0.7H), 4.75 (d, J=5.2 Hz, 1H), 4.56 (b, 1H), 4.43 (dd, J=7.6 Hz, J=5.2 Hz, 1H), 4.02-4.06 (m, 1.8H), 3.99 (d, J=4.8 Hz, 0.7H), 3.93 (d, J=4.0 Hz, 1H), 3.75 (m, 2H), 3.72 (s, 3H).

### Example 70. 7-((2R,3R,4S,5R)-5-((R)-(3,4-difluorophenyl)(hydroxy)methyl)-3,4-dihydroxytetrahydrofuran-2-yl)-1,7-dihydro-4H-pyrrolo[2,3-d]pyrimidin-4-one O-methyl oxime (70)

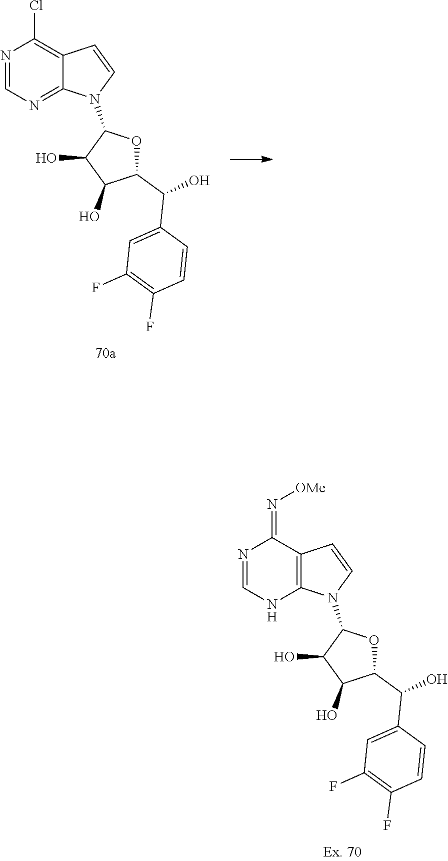 [SMILES: O[C@@H]1[C@H](O)[C@@H]([C@H](O)c2ccc(F)c(F)c2)O[C@H]1n1ccc2c(Cl)ncnc21 ; CO/N=c1/nc[nH]c2c1ccn2[C@@H]1O[C@H]([C@H](O)c2ccc(F)c(F)c2)[C@@H](O)[C@H]1O]

### a) Preparation of (2R,3S,4R,5R)-2-[(R)-(3,4-dichlorophenyl)-hydroxy-methyl]-5-(4-chloropyrrolo[2,3-d]pyrimidin-7-yl)tetrahydrofuran-3,4-diol (70a)

Compound 70a was prepared following a similar procedure as that of compound 20a except substituting (R)-[(3aR,4R,6R,6aR)-4-(4-chloropyrrolo[2,3-d]pyrimidin-7-yl)-2,2-dimethyl-3a,4,6,6a-tetrahydrofuro[3,4-d][1,3]dioxol-6-yl]-(4-chlorophenyl)methanol with (R)-[(3aR,4R,6R,6aR)-4-(4-chloropyrrolo[2,3-d]pyrimidin-7-yl)-2,2-dimethyl-3a,4,6,6a-tetrahydrofuro[3,4-d][1,3]dioxol-6-yl]-(3,4-difluorophenyl)methanol.

### b) Preparation of 7-((2R,3R,4S,5R)-5-((R)-(3,4-difluorophenyl)(hydroxy)methyl)-3,4-dihydroxytetrahydrofuran-2-yl)-1,7-dihydro-4H-pyrrolo[2,3-d]pyrimidin-4-one O-methyl oxime (70)

A 2 mL microwave vial with septum containing a solution of (2R,3R,4S,5R)-2-(4-chloro-7H-pyrrolo[2,3-d]pyrimidin-7-yl)-5-((R)-(3,4-difluorophenyl)(hydroxy)methyl)tetrahydrofuran-3,4-diol (70a) (50.3 mg, 0.130 mmol) and O-methylhydroxylamine hydrochloride (76 mg, 0.91 mmol) in IPA (0.5 mL) and N-ethyl-N-isopropyl-propan-2-amine (0.4 mL, 2.3 mmol) was sparged with Ar for 2 min, then heated in a microwave reactor for 1 h at 120° C. TLC showed minimal conversion of product. The vial was then heated conventionally at 110° C. for 2 d. The mixture was charged with IPA (1.5 mL), then O-methylhydroxylamine hydrochloride (90 .mg, 1.02 mmol), and N-ethyl-N-isopropyl-propan-2-amine (0.3 mL, 1.72 mmol). The vial was blanketed with Ar, then heated at 110° C. for 3 d. The reaction mixture was concentrated under reduced pressure and heat (50° C.), then attempted purification by FCC (30 g C18, 5→25% MeCN in H₂O, wet-loaded in H₂O+DMSO). Fractions containing product by HPLC were combined, concentrated, neutralized with Amberlite IRA-67 resin, filtered, and repurified by FCC (12 g SiO₂, 0→4% MeOH in DCM, wet-loaded in eluent). Fraction containing pure product were combined, concentrated under reduced pressure, and twice co-evaporated with 1N HCl_{(aq)} and methanol to yield the HCl salt of (Z)-7-((2R,3R,4S,5R)-5-((R)-(3,4-difluorophenyl)(hydroxy)methyl)-3,4-dihydroxytetrahydrofuran-2-yl)-1,7-dihydro-4H-pyrrolo[2,3-d]pyrimidin-4-one O-methyl oxime (70) (17.9 mg, 0.0398 mmol, 31.5% yield) as an off-white powder. Rf=0.20 (6% MeOH in DCM); LRMS (ESI) m/z calcd for [M+H]⁺ C₁₈H₁₉F₂N₄O₅: 409.13. Found: 409.2; ¹H NMR (400 MHz, DMSO-d₆+D₂O) δ 8.18 (s, 1H), 7.66 (s, 1H), 7.46-7.31 (m, 2H), 7.28-7.19 (m, 1H), 6.71 (s, 1H), 6.06 (d, J=7.6 Hz, 1H), 4.77 (d, J=5.4 Hz, 1H), 4.48 (dd, J=7.7, 5.0 Hz, 1H), 4.09 (dd, J=5.0, 1.4 Hz, 1H), 3.98 (dd, J=5.3, 1.3 Hz, 1H), 3.84 (s, 3H).

### Example 71. 7-((2R,3R,4S,5R)-5-((R)-(4-chlorophenyl)(hydroxy)methyl)-3,4-dihydroxytetrahydrofuran-2-yl)-2-methyl-1,7-dihydro-4H-pyrrolo[2,3-d]pyrimidin-4-one oxime (71)

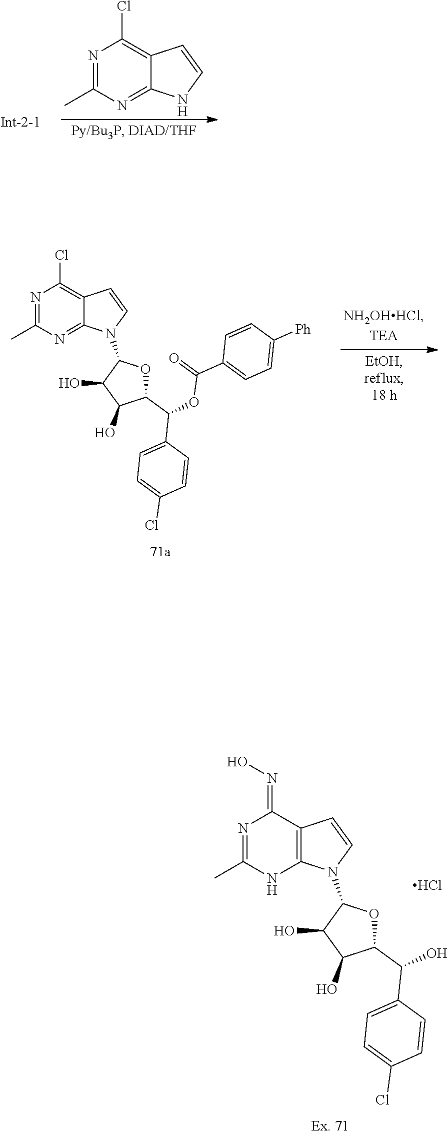 [SMILES: Cc1nc(Cl)c2cc[nH]c2n1 ; Cc1nc(Cl)c2ccn([C@@H]3O[C@H]([C@H](OC(=O)c4ccc(-c5ccccc5)cc4)c4ccc(Cl)cc4)[C@@H](O)[C@H]3O)c2n1 ; Cc1n/c(=N\O)c2ccn([C@@H]3O[C@H]([C@H](O)c4ccc(Cl)cc4)[C@@H](O)[C@H]3O)c2[nH]1 ; Cl]

### a) Preparation of [(R)-[(2S,3S,4R,5R)-5-(4-chloro-2-methyl-pyrrolo [2,3-d]pyrimidin-7-yl)-3,4-dihydroxy-tetrahydrofuran-2-yl]-(4-chlorophenyl)methyl]4-phenylbenzoate (71a)

To a solution of 4-chloro-2-methyl-7H-pyrrolo[2,3-d]pyrimidine (190.08 mg, 1.11 mmol) and pyridine (0.09 mL, 1.11 mmol) in dry THF (5 mL) was added tributylphosphane (0.56 mL, 2.22 mmol) and DIAD (0.46 mL, 2.33 mmol) at 30° C. Then [(R)-(4-chlorophenyl)-[(2S,3S,4R)-3,4,5-trihydroxytetrahydrofuran-2-yl]methyl]4-phenylbenzoate (Int-2-1) (500 mg, 1.11 mmol) in dry THF (10 mL) was added in one portion. The reaction mixture was stirred at 30° C. for 1 h. LCMS showed the reaction was completed. The solution was purified by prep-HPLC (0.1% TFA) eluting with H₂O:CH₃CN from 90:10 to 5:95 to give [(R)-[(2S,3S,4R,5R)-5-(4-chloro-2-methyl-pyrrolo [2,3-d]pyrimidin-7-yl)-3,4-dihydroxy-tetrahydrofuran-2-yl]-(4-chlorophenyl)methyl]4-phenylbenzoate (71a) (140 mg, 0.2324 mmol, 20.907% yield) as pale yellow solid. LCMS [M+H]: 590.3.

### b) Preparation of 7-((2R,3R,4S,5R)-5-((R)-(4-chlorophenyl)(hydroxy)methyl)-3,4-dihydroxytetrahydrofuran-2-yl)-2-methyl-1,7-dihydro-4H-pyrrolo[2,3-d]pyrimidin-4-one oxime (71)

To a solution of [(R)-[(2S,3S,4R,5R)-5-(4-chloro-2-methyl-pyrrolo[2,3-d]pyrimidin-7-yl)-3,4-dihydroxy-tetrahydrofuran-2-yl]-(4-chlorophenyl)methyl] 4-phenylbenzoate (71a) (210 mg, 0.36 mmol) in ethanol (5 mL) was added hydroxylamine hydrochloride (0.22 mL, 7.11 mmol) and TEA (1.07 g, 10.67 mmol). The reaction mixture was heated to 90° C. and stirred for 18 hh. The reaction mixture was concentrated under reduced pressure. The residue was washed with water, extracted with EtOAc. The combined organic layers were washed with brine, dried over anhydrous Na₂SO₄. The solvent was concentrated under reduced pressure to give impure product, which was purified by prep-HPLC (0.1% TFA) eluting with H₂O:CH₃CN from 95:5 to 5:95. The product was dissolved in 1M HCl, lyophilized to give 7-[(2R,3R,4S,5R)-5-[(R)-(4-chlorophenyl)-hydroxy-methyl]-3,4-dihydroxy-tetrahydrofuran-2-yl]-2-methyl-1H-pyrrolo[2,3-d]pyrimidin-4-one oxime hydrochloride (71) (21.3 mg, 0.0471 mmol, 13.2% yield) as yellow solid. LCMS [M+H]: 407.3. ¹H NMR (400 MHz, DMSO-d6) δ 11.11 (s, 1H), 7.65 (d, J=2.8 Hz, 1H), 7.35-7.41 (m, 4H), 6.90 (d, J=3.2 Hz, 1H), 6.06 (d, J=7.2 Hz, 1H), 4.76 (d, J=5.2 Hz, 1H), 4.48-4.51 (m, 1H), 4.11 (d, J=4.4 Hz, 1H), 3.97 (d, J=5.2 Hz, 1H), 2.60 (s, 3H). ¹H NMR (400 MHz, DMSO-d6+D₂O) δ 7.65 (d, J=3.6 Hz, 1H), 7.35-7.41 (m, 4H), 6.82 (d, J=3.6 Hz, 1H), 6.07 (d, J=8.0 Hz, 1H), 4.75 (d, J=5.2 Hz, 1H), 4.49-4.51 (m, 1H), 4.12 (d, J=5.2 Hz, 1H), 3.99 (d, J=5.2 Hz, 1H), 2.61 (s, 3H).

### Example 72. 7-((2R,3R,4S,5R)-5-((R)-(4-chlorophenyl)(hydroxy)methyl)-3,4-dihydroxytetrahydrofuran-2-yl)-5-fluoro-1,7-dihydro-4H-pyrrolo[2,3-d]pyrimidin-4-one oxime (72)

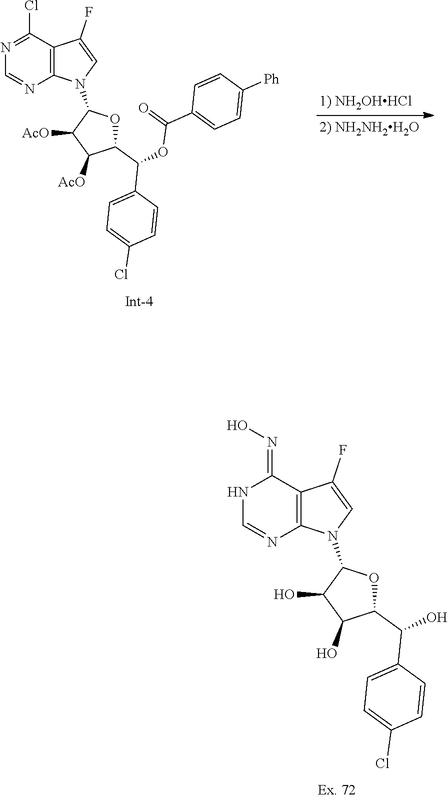 [SMILES: CC(=O)O[C@H]1[C@@H](OC(C)=O)[C@H](n2cc(F)c3c(Cl)ncnc32)O[C@@H]1[C@H](OC(=O)c1ccc(-c2ccccc2)cc1)c1ccc(Cl)cc1 ; O/N=c1\[nH]cnc2c1c(F)cn2[C@@H]1O[C@H]([C@H](O)c2ccc(Cl)cc2)[C@@H](O)[C@H]1O]

To a solution of [(R)-(4-chlorophenyl)-[(2R,3R,4R,5R)-3,4-diacetoxy-5-(4-chloro-5-fluoro-pyrrolo [2,3-d]pyrimidin-7-yl)tetrahydrofuran-2-yl]methyl] 4-phenylbenzoate (Int-4) (185.0 mg, 0.24 mmol) in ethanol (5 mL) was added hydroxylamine hydrochloride (135.0 mg, 1.93 mmol) and triethylamine (0.4 mL, 2.87 mmol). The mixture was heated to reflux and stirred for 3 hh. LCMS showed the material was consumed, but the protection groups were remained. The solvent was removed in vacuo, and the residue was dissolved in 1,4-dioxane (2 mL). Then hydrazine hydrate (1.01 mL, 20.58 mmol) was added. The mixture was stirred at 24° C. for 16 hh. LCMS showed the benzoate was removed. The mixture was diluted by EtOAc (25 mL), dried over anhydrous Na₂SO₄, filtered, concentrated in vacuo. The residue was purified by prep-HPLC (0.1% TFA eluting with H₂O:CH₃CN from 90:10 to 5:95), then 0.05 mL of conc. HCl was added. The mixture was lyophilized to afford 7-[(2R,3R,4S,5R)-5-[(R)-(4-chlorophenyl)-hydroxy-methyl]-3,4-dihydroxy-tetrahydrofuran-2-yl]-5-fluoro-1H-pyrrolo[2,3-d]pyrimidin-4-one oxime hydrochloride (72) (17.5 mg, 0.037 mmol, 15.5% yield) as white solid. ¹H NMR (400 MHz, DMSO-d₆) δ 8.14 (s, 1H), 7.63 (s, 1H), 7.36-7.42 (m, 4H), 6.10 (d, J=7.2 Hz, 1H), 4.76-4.77 (m, 1H), 4.38-4.41 (m, 1H), 4.09-4.11 (m, 1H), 3.96-3.97 (m, 1H). LCMS [M+H]: m/z 411.2.

### Example 73. (2R,3S,4R,5R)-2-((R)-(4-chlorophenyl)(hydroxy)methyl)-5-(5-fluoro-4-hydrazineylidene-1,4-dihydro-7H-pyrrolo[2,3-d]pyrimidin-7-yl)tetrahydrofuran-3,4-diol (73)

To a solution of [(R)-(4-chlorophenyl)-[(2R,3R,4R,5R)-3,4-diacetoxy-5-(4-chloro-5-fluoro-pyrrolo[2,3-d]pyrimidin-7-yl)tetrahydrofuran-2-yl]methyl] 4-phenylbenzoate (Int-4) (68.97 mg, 0.09 mmol) in 1,4-dioxane (2 mL) was added hydrazine monohydrate (9 μL, 0.29 mmol). The mixture was stirred at 24° C. for 1 h. LCMS showed the material was consumed, but biphenyl was remained. More hydrazine monohydrate (2 mL, 64 mmol) was added, and the mixture was stirred at 24° C. for 1 h. LCMS show the reaction was complete and EtOAc (5 mL) was added. The mixture was dried over Na₂SO₄, and filtered. The filtrate was concentrated in vacuo. The residue was purified by prep-HPLC (0.1% TFA) eluting with H₂O:CH₃CN from 90:10 to 5:95, then 0.1 mL of conc. HCl was added and the resulting mixture was lyophilized to afford 7-[(2R,3R,4S,5R)-5-[(R)-(4-chlorophenyl)-hydroxy-methyl]-3,4-dihydroxy-tetrahydrofuran-2-yl]-5-fluoro-1H-pyrrolo[2,3-d]pyrimidin-4-one hydrazone hydrochloride (73) (12 mg, 0.0248 mmol, 28% yield) as a white solid. ¹H NMR (400 MHz, DMSO-d₆) δ 8.39 (s, 1H), 7.70 (s, 1H), 7.30-7.45 (m, 4H), 6.16 (d, J=7.2 Hz, 1H), 4.76-4.77 (m, 1H), 4.43-4.46 (m, 1H), 4.09-4.11 (m, 1H), 3.96-3.97 (m, 1H). LCMS [M+H]: 416.1.

### Example 74. 7-((2R,3R,4S)-5-((R)-(4-chlorophenyl)(hydroxy)methyl)-3,4-dihydroxytetrahydrofuran-2-yl)-5-fluoro-1,7-dihydro-4H-pyrrolo[2,3-d]pyrimidin-4-one O-methyl oxime (74)

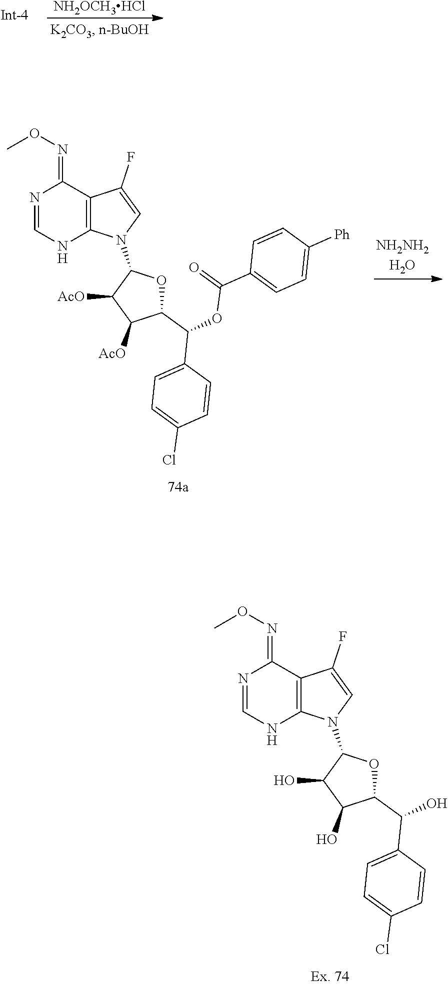 [SMILES: "NH2OCH3·HCl" ; CO/N=c1\nc[nH]c2c1c(F)cn2[C@@H]1O[C@H]([C@H](OC(=O)c2ccc(-c3ccccc3)cc2)c2ccc(Cl)cc2)[C@@H](OC(C)=O)[C@H]1OC(C)=O ; CO/N=c1\nc[nH]c2c1c(F)cn2[C@@H]1O[C@H]([C@H](O)c2ccc(Cl)cc2)[C@@H](O)[C@H]1O]

### a) Preparation of [(R)-(4-chlorophenyl)-[(2R,3R,4R,5R)-3,4-diacetoxy-5-[5-fluoro-4-(methoxyamino)pyrrolo[2,3-d]pyrimidin-7-yl]tetrahydrofuran-2-yl]methyl] 4-phenylbenzoate (74a)

To a solution of [(R)-(4-chlorophenyl)-[(2R,3R,4R,5R)-3,4-diacetoxy-5-(4-chloro-5-fluoro-pyrrolo[2,3-d]pyrimidin-7-yl)tetrahydrofuran-2-yl]methyl] 4-phenylbenzoate (Int-4) (120 mg, 0.17 mmol) in 1-butanol (5 mL) was added methoxyammonium chloride (120 mg, 1.42 mmol) and potassium carbonate (120.0 mg, 0.86 mmol). The mixture was stirred at 100° C. for 4.5 h. The mixture was diluted with EtOAc (40 mL), washed with brine (20 mL), dried over Na₂SO₄ and filtered. The filtrate was concentrated in vacuo to afford [(R)-(4-chlorophenyl)-[(2R,3R,4R,5R)-3,4-diacetoxy-5-[5-fluoro-4-(methoxyamino)pyrrolo[2,3-d]pyrimidin-7-yl]tetrahydrofuran-2-yl]methyl] 4-phenylbenzoate (74a) (105 mg, 0.090 mmol, 51.8% yield) as yellow solid, which used in the next step without further purification. LCMS [M+H]: m/z 689.3.

### b) Preparation of (2R,3S,4R,5R)-2-[(R)-(4-chlorophenyl)-hydroxy-methyl]-5-[(4Z)-5-fluoro-4-methoxyimino-1H-pyrrolo[2,3-d]pyrimidin-7-yl]tetrahydrofuran-3,4-diol hydrochloride (74)

To a solution of [(R)-(4-chlorophenyl)-[(2R,3R,4R,5R)-3,4-diacetoxy-5-[5-fluoro-4-(methoxyamino)pyrrolo[2,3-d]pyrimidin-7-yl]tetrahydrofuran-2-yl]methyl] 4-phenylbenzoate (74a) (159 mg, 0.15 mmol) in ethanol (2 mL) was added hydrazine hydrate (1 mL, 20.6 mmol). The mixture was stirred at 24° C. for 2 hh. The mixture was diluted with EtOAc (30 mL), washed with brine (25 mL), dried over Na₂SO₄ and filtered. The filtrate was concentrated in vacuo. The residue was first purified prep-TLC, then further purified by prep-HPLC (0.1% TFA) eluting with H₂O:CH₃CN from 90:10 to 5:95. The product was treated with 1N HCl to afford (2R,3 S,4R,5R)-2-[(R)-(4-chlorophenyl)-hydroxy-methyl]-5-[(4Z)-5-fluoro-4-methoxyimino-1H-pyrrolo[2,3-d]pyrimidin-7-yl]tetrahydrofuran-3,4-diol hydrochloride (74) (7 mg, 0.015 mmol, 9.8% yield) as white solid. ¹H NMR (400 MHz, DMSO-d6) δ 7.58 (s, 1H), 7.35-7.41 (m, 4H), 7.21 (s, 1H), 5.94 (d, J=6.8 Hz, 1H), 4.71-4.73 (m, 1H), 4.33-4.37 (m, 1H), 3.99-4.05 (m, 1H), 3.90-3.92 (m, 1H), 3.74 (s, 3H). LCMS [M+H]: m/z 411.2.

### Example 75. 7-((2R,3R,4S,5R)-3,4-dihydroxy-5-((R)-hydroxy(4-(trifluoromethyl)phenyl)methyl)tetrahydrofuran-2-yl)-1,7-dihydro-4H-pyrrolo[2,3-d]pyrimidin-4-one O-methyl oxime (75)

Example 75 was prepared following the similar procedures as those of Example 74 except substituting Int-4 with Int-1. LCMS [M+H]: 441.3. ¹H NMR (400 MHz, DMSO-d₆) δ 8.26 (s, 1H), 7.72 (d, J=2.4 Hz, 1H), 7.60-7.68 (m, 4H), 6.84 (s, 1H), 6.09 (d, J=8.0 Hz, 1H), 4.88 (d, J=5.2 Hz, 1H), 4.50-4.54 (m, 1H), 4.13 (d, J=4.8 Hz, 1H), 4.03 (d, J=4.8 Hz, 1H), 3.86 (s, 3H). ¹H NMR (400 MHz, DMSO-d₆+D₂O) δ 8.30 (s, 1H), 7.72 (d, J=3.6 Hz, 1H), 7.61-7.69 (m, 4H), 6.79 (d, J=3.6 Hz, 1H), 6.11 (d, J=8.0 Hz, 1H), 4.87 (d, J=5.2 Hz, 1H), 4.50-4.54 (m, 1H), 4.13 (d, J=4.8 Hz, 1H), 4.05 (d, J=5.2 Hz, 1H), 3.87 (s, 3H).

### Example 76. (2R,3R,4S,5R)-2-((Z)-4-hydrazineylidene-1,4-dihydro-7H-pyrrolo[2,3-d]pyrimidin-7-yl)-5-((R)-hydroxy(4-(trifluoromethyl)phenyl)methyl)tetrahydrofuran-3,4-diol (76)

Example 76 was prepared following the similar procedures as those of Example 73 except substituting Int-4 with Int-1. LCMS [M+H]: 426.4. ¹H NMR (400 MHz, DMSO-d6) δ 11.13 (s, 1H), 8.31 (s, 1H), 7.72 (d, J=3.6 Hz, 1H), 7.61-7.68 (m, 4H), 6.97 (d, J=3.2 Hz, 1H), 6.11 (d, J=7.6 Hz, 1H), 4.88 (d, J=5.2 Hz, 1H), 4.52-4.56 (m, 1H), 4.14 (d, J=4.8 Hz, 1H), 4.03 (d, J=5.2 Hz, 1H). ¹H NMR (400 MHz, DMSO-d₆+D₂O) δ 8.32 (s, 1H), 7.61-7.72 (m, 5H), 6.93 (d, J=3.6 Hz, 1H), 6.12 (d, J=7.6 Hz, 1H), 4.87 (d, J=5.2 Hz, 1H), 4.52-4.56 (m, 1H), 4.13 (d, J=4.8 Hz, 1H), 4.05 (d, J=5.6 Hz, 1H).

### Example 77. 7-((2R,3R,4S,5R)-5-((R)-(4-chlorophenyl)(hydroxy)methyl)-3,4-dihydroxytetrahydrofuran-2-yl)-5-methyl-1,7-dihydro-4H-pyrrolo[2,3-d]pyrimidin-4-one oxime (77)

Example 77 was prepared following the similar procedures as those of Example 37 except for substituting 4-chloro-7H-pyrrolo[2,3-d]pyrimidine with 4-chloro-5-methyl-7H-pyrrolo[2,3-d]pyrimidine. LCMS [M+H]: 407.1. ¹H NMR (400 M Hz, DMSO-d6): δ 13.55 (s, 1H), 11.02 (s, 1H), 8.26 (s, 1H), 7.36-7.44 (m, 5H), 6.07 (d, J=7.2 Hz, 1H), 4.76 (d, J=5.2 Hz, 1H), 4.38-4.42 (m, 1H), 4.08 (d, J=4.8 Hz, 1H), 3.99 (d, J=4.0 Hz, 1H), 2.37 (s, 3H). ¹H NMR (400 M Hz, DMSO-d6+D₂O): δ 8.29 (s, 1H), 7.36-7.43 (m, 5H), 6.08 (d, J=7.2 Hz, 1H), 4.76 (d, J=5.2 Hz, 1H), 4.39-4.43 (m, 1H), 4.08 (d, J=4.8 Hz, 1H), 4.01 (d, J=4.0 Hz, 1H), 2.37 (s, 3H).

### Example 78. 7-((2R,3R,4S,5R)-5-((R)-(3-fluoro-4-(trifluoromethyl)phenyl)(hydroxy)methyl)-3,4-dihydroxytetrahydrofuran-2-yl)-1,7-dihydro-4H-pyrrolo[2,3-d]pyrimidin-4-one O-methyl oxime (78)

Example 78 was prepared following the same procedure as Example 75 except for substituting (4-(trifluoromethyl)phenyl)boronic acid with (3-fluoro-4-(trifluoromethyl)phenyl)boronic acid. LCMS [M+H]: 459.2. ¹H NMR (400 M Hz, DMSO-d6+D₂O): δ 8.13 (s, 1H), 7.73 (t, J=7.6 Hz, 1H), 7.59 (d, J=3.4 Hz, 1H), 7.48-7.44 (m, 2H), 6.66 (d, J=3.2 Hz, 1H), 6.05 (d, J=7.4 Hz, 1H), 4.88 (d, J=4.8 Hz, 1H), 4.52-4.49 (m, 1H), 4.11-4.10 (m, 1H), 4.05-4.04 (m, 1H), 3.83 (s, 3H).

### Example 79. 7-((2R,3R,4S)-5-((R)-1-(4-chlorophenyl)-2,2,2-trifluoro-1-hydroxyethyl)-3,4-dihydroxytetrahydrofuran-2-yl)-1,7-dihydro-4H-pyrrolo[2,3-d]pyrimidin-4-one O-methyl oxime (79)

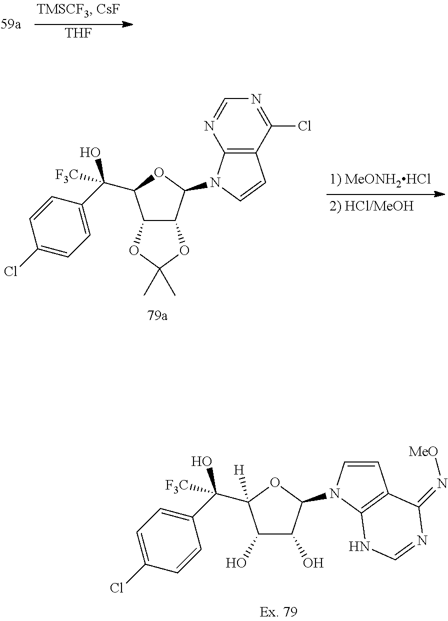 [SMILES: CC1(C)O[C@@H]2[C@H](O1)[C@@H]([C@](O)(c1ccc(Cl)cc1)C(F)(F)F)O[C@H]2n1ccc2c(Cl)ncnc21 ; CO/N=c1/nc[nH]c2c1ccn2[C@@H]1O[C@H]([C@](O)(c2ccc(Cl)cc2)C(F)(F)F)[C@@H](O)[C@H]1O ; "THF"]

### a) Preparation of (R)-1-((3aR,4S,6R,6aR)-6-(4-chloro-7H-pyrrolo[2,3-d]pyrimidin-7-yl)-2,2-dimethyltetrahydrofuro[3,4-d][1,3]dioxol-4-yl)-1-(4-chlorophenyl)-2,2,2-trifluoroethan-1-ol (79a)

To a solution of compound 59a (50 mg, 115.14 umol, 1 eq.) in THE (2 mL) was added CsF (87.45 mg, 575.68 umol, 21.22 uL, 5 eq.), TMSCF₃ (32.74 mg, 230.27 umol, 2 eq.). The mixture was stirred at −20° C. for 3 h. LC-MS showed compound 4 was consumed completely and one main peak with desired MS was detected. The reaction was quenched by H₂O (4 mL), and extracted with EtOAc (4 mL*3), and the organic phase was concentrated in vacuo. The residue was purified by prep-TLC (SiO2, Petroleum ether:Ethyl acetate=5:1). Compound 79a (10 mg) was obtained as a white solid (and 8 mg of the diastereomer). 1H NMR (400 MHz, CHLOROFORM-d) δ=8.63 (s, 1H), 8.07 (s, 1H), 7.65-7.53 (m, 2H), 7.36 (d, J=8.3 Hz, 2H), 7.26-7.20 (m, 1H), 6.57 (d, J=3.5 Hz, 1H), 5.78 (d, J=5.0 Hz, 1H), 5.09-4.90 (m, 2H), 4.48 (br d, J=6.4 Hz, 1H), 1.48 (s, 3H), 1.09-1.04 (m, 3H); LCMS: (M+H+): 503.9, 505.9; TLC (Petroleum ether:Ethyl acetate=5:1) R_{f}=0.43.

### b) Preparation of 7-((2R,3R,4S)-5-((R)-1-(4-chlorophenyl)-2,2,2-trifluoro-1-hydroxyethyl)-3,4-dihydroxytetrahydrofuran-2-yl)-1,7-dihydro-4H-pyrrolo[2,3-d]pyrimidin-4-one O-methyl oxime (79)

Example 79 was prepared using the similar procedures as those of Example 60 except for substituting (R)-1-((3aR,4S,6R,6aR)-6-(4-chloro-7H-pyrrolo[2,3-d]pyrimidin-7-yl)-2,2-dimethyltetrahydrofuro[3,4-d][1,3]dioxol-4-yl)-1-(4-chlorophenyl)ethan-1-ol (59b) with (R)-1-((3aR,4S,6R,6aR)-6-(4-chloro-7H-pyrrolo[2,3-d]pyrimidin-7-yl)-2,2-dimethyltetrahydrofuro[3,4-d][1,3]dioxol-4-yl)-1-(4-chlorophenyl)-2,2,2-trifluoroethan-1-ol (79a). ¹H NMR (400 MHz, DMSO-d6) δ=8.20 (s, 1H), 7.67 (d, J=8.4 Hz, 2H), 7.59 (br d, J=3.1 Hz, 1H), 7.55 (d, J=8.6 Hz, 2H), 6.71 (br d, J=3.1 Hz, 1H), 5.97 (d, J=8.2 Hz, 1H), 4.66 (s, 1H), 4.47 (br dd, J=5.5, 7.9 Hz, 1H), 3.84 (s, 3H), 3.60 (d, J=5.5 Hz, 1H); ¹H NMR (400 MHz, DMSO-d6+D₂O) δ=8.22 (s, 1H), 7.65 (d, J=8.8 Hz, 2H), 7.60 (d, J=3.5 Hz, 1H), 7.55 (d, J=8.8 Hz, 2H), 6.69 (d, J=3.5 Hz, 1H), 5.97 (d, J=8.2 Hz, 1H), 4.65 (s, 1H), 4.45 (dd, J=5.4, 8.0 Hz, 1H), 3.84 (s, 3H); LCMS: (M+H⁺): 475.0; HPLC purity: 97.36%;

### Example 80. 7-((2R,3R,4S,5R)-5-((R)-(4-chloro-3-methylphenyl)(hydroxy)methyl)-3,4-dihydroxytetrahydrofuran-2-yl)-1,7-dihydro-4H-pyrrolo[2,3-d]pyrimidin-4-one O-methyl oxime (80)

Example 80 was prepared following the same procedure as Example 75 except for substituting (4-(trifluoromethyl)phenyl)boronic acid with (3-methyl-4-(trifluoromethyl)phenyl)boronic acid. LCMS [M+H]: 421.3; ¹H NMR (400 MHz, DMSO-d6+D₂O) δ 8.08 (s, 1H), 7.49 (d, J=3.4 Hz, 1H), 7.32-7.30 (m, 2H), 7.19 (d, J=8.2 Hz, 1H), 6.64 (d, J=3.5 Hz, 1H), 6.00 (d, J=7.6 Hz, 1H), 4.70 (d, J=4.8 Hz, 1H), 4.48-4.45 (m, 1H), 4.08-4.07 (m, 1H), 4.01-3.99 (m, 1H), 3.80 (s, 3H), 2.26 (s, 3H).

### Example 81. (2R,3S,4R,5R)-2-((R)-(3,4-dichlorophenyl)(hydroxy)methyl)-5-(4-(2-methylhydrazineylidene)-1,4-dihydro-7H-pyrrolo[2,3-d]pyrimidin-7-yl)tetrahydrofuran-3,4-diol (81)

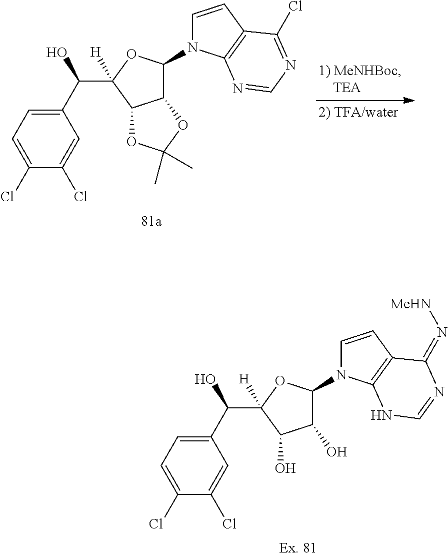 [SMILES: CC1(C)O[C@H]2[C@@H](O1)[C@H](n1ccc3c(Cl)ncnc31)O[C@@H]2[C@H](O)c1ccc(Cl)c(Cl)c1 ; CN/N=c1/nc[nH]c2c1ccn2[C@@H]1O[C@H]([C@H](O)c2ccc(Cl)c(Cl)c2)[C@@H](O)[C@H]1O]

### a) Preparation of (R)-[(3aR,4R,6R,6aR)-4-(4-chloropyrrolo[2,3-d]pyrimidin-7-yl)-2,2-dimethyl-3a,4,6,6a-tetrahydrofuro[3,4-d][1,3]dioxol-6-yl]-(3,4-dichlorophenyl)methanol (81a)

Compound 81a was prepared following the same procedure as that of Int-3 except for substituting [(3aR,4R,6S,6aS)-4-(4-chloropyrrolo[2,3-d]pyrimidin-7-yl)-2,2-dimethyl-3a,4,6,6a-tetrahydrofuro[3,4-d][1,3]dioxol-6-yl]-(4-chloro-3-fluoro-phenyl)methanone (Int-3-1) with [(3aR,4R,6S,6aS)-4-(4-chloropyrrolo[2,3-d]pyrimidin-7-yl)-2,2-dimethyl-3a,4,6,6a-tetrahydrofuro[3,4-d][1,3]dioxol-6-yl]-(3,4-dichlorophenyl)methanone.

### b) Preparation of (2R,3S,4R,5R)-2-((R)-(3,4-dichlorophenyl)(hydroxy)methyl)-5-(4-(2-methylhydrazineylidene)-1,4-dihydro-7H-pyrrolo[2,3-d]pyrimidin-7-yl)tetrahydrofuran-3,4-diol (81)

A microwave tube containing a mixture of (R)-[(3aR,4R,6R,6aR)-4-(4-chloropyrrolo[2,3-d]pyrimidin-7-yl)-2,2-dimethyl-3a,4,6,6a-tetrahydrofuro[3,4-d][1,3]dioxol-6-yl]-(3,4-dichlorophenyl)methanol (81a) (110 .mg, 0.2300 mmol) was charged with 1-Boc-1-methylhydrazine (0.5 mL, 3.27 mmol) and Triethylamine; TEA (120.51 uL, 0.8600 mmol). Then, it was blanketed with N₂, heated at 95° C. After 24 h LCMS showed 1:1 ratio between the final compound and the starting material. The reaction was purged with N₂ and stirred for another 21 h at 95° C. LCMS showed 2:1 ratio. Another equiv of Et₃N and 3.7 equiv of 1-Boc-1-methylhydrazine were added and the reaction was stirred for 30 min at 95° C. in the microwave. LCMS showed the same ratio as before so the reaction was stirred at 100° C. for another 3 h. This time the ratio was 2.5:1 so the reaction was stirred for another 16 h at 100° C. Although there still remained some starting material, the crude was concentrated and treated with 1.5 mL of a 9:1 (TFA:water) solution for 2 h (LCMS showed no more starting material). The crude was concentrated, dissolved in DMSO, loaded onto a 30 g C18 column and purified using H₂O/ACN (2 min 5% AcCN then ramp up for 5 min until 40%, then 8 min at 40% and finally ramp up to 80%) to give (2R,3S,4R,5R)-2-[(R)-(3,4-dichlorophenyl)-hydroxy-methyl]-5-[4-(methylhydrazono)-1H-pyrrolo[2,3-d]pyrimidin-7-yl]tetrahydrofuran-3,4-diol TFA salt (81) (48 mg, 0.095 mmol, 41% yield). ¹H NMR (400 MHz, Methanol-d₄) δ 8.22 (s, 1H), 7.64 (d, J=3.8 Hz, 1H), 7.57 (d, J=1.9 Hz, 1H), 7.47 (d, J=8.3 Hz, 1H), 7.34 (dd, J=2.0, 8.3 Hz, 1H), 6.84 (d, J=3.8 Hz, 1H), 6.23 (d, J=6.4 Hz, 1H), 4.59 (dd, J=5.2, 6.4 Hz, 1H), 4.26 (dd, J=2.9, 5.2 Hz, 1H), 4.23 (t, J=3.2 Hz, 1H), 2.78 (s, 3H).

### Example 82. 7-((2R,3R,4S,5R)-5-((R)-(3,4-dichlorophenyl)(hydroxy)methyl)-3,4-dihydroxytetrahydrofuran-2-yl)-3,7-dihydro-4H-pyrrolo[2,3-d]pyrimidin-4-one O-ethyl oxime (82)

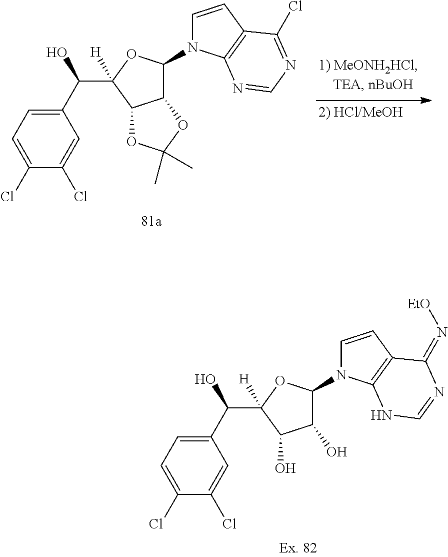 [SMILES: CC1(C)O[C@H]2[C@@H](O1)[C@H](n1ccc3c(Cl)ncnc31)O[C@@H]2[C@H](O)c1ccc(Cl)c(Cl)c1 ; CCO/N=c1/nc[nH]c2c1ccn2[C@@H]1O[C@H]([C@H](O)c2ccc(Cl)c(Cl)c2)[C@@H](O)[C@H]1O]

To a solution of (R)-[(3aR,4R,6R,6aR)-4-(4-chloropyrrolo[2,3-d]pyrimidin-7-yl)-2,2-dimethyl-3a,4,6,6a-tetrahydrofuro[3,4-d][1,3]dioxol-6-yl]-(3,4-dichlorophenyl)methanol (81a) (102 .mg, 0.22 mmol) in 1-Butanol (1 mL) was added the Triethylamine; TEA (0.18 mL, 1.3 mmol) and ethoxyamine hydrochloride (108.95 mg, 1.08 mmol). The reaction was sparged with nitrogen and heated in an oil bath for 16 h at 110° C. The reaction mixture was concentrated and the crude was dissolved in 1 mL of methanol and treated with few drops of conc HCl. The reaction was stirred for 2 h, concentrated, the crude redissolved back in 1 mL of MeOH and 1 mL of water, treated with Amberlite IRA 67 and stirred for 30 mins. The reaction mixture was filtered, concentrated and the crude purified by silica gel chromatography using 4 g Agela column and gradient of 0-10% MeOH in DCM. The product was treated with 1N HCl to give (2R,3S,4R,5R)-2-[(R)-(3,4-dichlorophenyl)-hydroxy-methyl]-5-[4-ethoxyimino-3H-pyrrolo[2,3-d]pyrimidin-7-yl]tetrahydrofuran-3,4-diol hydrochloride (82) (9 mg, 0.02 mmol, 9% yield). ¹H NMR (400 MHz, DMSO-d₆) δ 8.15 (d, J=7.7 Hz, 1H), 7.68-7.53 (m, 4H), 7.38 (dd, J=2.0, 8.4 Hz, 1H), 6.69 (s, 1H), 6.05 (d, J=7.5 Hz, 1H), 4.79 (d, J=5.4 Hz, 1H), 4.50 (dd, J=4.9, 7.6 Hz, 1H), 4.13-3.94 (m, 4H), 1.30 (t, J=7.0 Hz, 3H).

### Example 83. 7-((2R,3R,4S)-5-((R)-1-(4-chlorophenyl)-1-hydroxyethyl)-3,4-dihydroxytetrahydrofuran-2-yl)-1,7-dihydro-4H-pyrrolo[2,3-d]pyrimidin-4-one O-ethyl oxime (83)

Example 83 was prepared following the same procedure as Example 60 except for substituting MeONH₂.HCl with EtONH₂.HCl. ¹H NMR (400 MHz, DMSO-d6) δ=8.22 (br s, 1H), 7.67 (br s, 1H), 7.55 (d, J=8.6 Hz, 2H), 7.40 (d, J=8.6 Hz, 2H), 6.76 (br s, 1H), 6.04 (br d, J=7.9 Hz, 1H), 4.47-4.38 (m, 1H), 4.09-4.03 (m, 3H), 3.71 (br d, J=5.1 Hz, 1H), 1.42 (s, 3H), 1.31 (t, J=6.9 Hz, 3H); ¹H NMR (400 MHz, DMSO-d6+D₂O) δ=8.20 (s, 1H), 7.64 (d, J=3.3 Hz, 1H), 7.52 (d, J=8.6 Hz, 2H), 7.39 (d, J=8.6 Hz, 2H), 6.70 (d, J=3.7 Hz, 1H), 6.02 (d, J=7.7 Hz, 1H), 4.41 (dd, J=5.1, 7.9 Hz, 1H), 4.07-4.01 (m, 3H), 3.70 (br d, J=5.3 Hz, 1H), 1.40 (s, 3H), 1.29 (t, J=6.9 Hz, 3H); LCMS: (M+H⁺): 435.1

### Example 92. (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol (92)

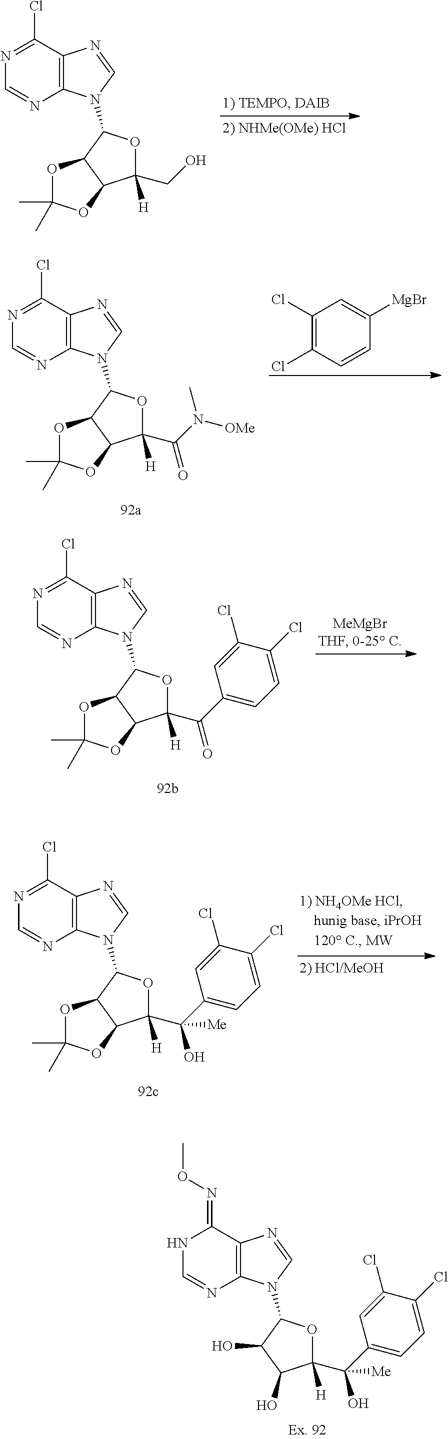

### a) Synthesis of (3aR,4R,6S,6aS)-4-(6-chloropurin-9-yl)-N-methoxy-N,2,2-trimethyl-3a,4,6,6a-tetrahydrofuro[3,4-d][1,3]dioxole-6-carboxamide (92a)

A mixture of 6-Chloro-9-(2,3-O-isopropylidene-beta-D-ribofuranosyl)-9H-purine (5 .g, 14.69 mmol) in MeCN (15 mL) and Water (15 mL) was cooled to 0° C. (Diacetoxyiodo)benzene (10.4 g, 32.32 mmol) and TEMPO (462 mg, 2.94 mmol) was added portionwise. The resulting mixture was stirred at RT overnight. TLC (9:1 DCM/MeOH) showed completion of the reaction.

The solid was filtered and quickly rinsed with EtOAc to give (3aR,4R,6S,6aS)-4-(6-chloropurin-9-yl)-2,2-dimethyl-3a,4,6,6a-tetrahydrofuro[3,4-d][1,3]dioxole-6-carboxylic acid (2.68 g, 7.87 mmol, 53.5% yield) as an off-white solid. The aq. layer of the filtrates was extracted with EtOAc and the combined organic layers were washed with sat. aq. sodium thiosulfate, water, brine, dried over sodium sulfate, filtered and concentrated to give (3aR,4R,6S,6aS)-4-(6-chloropurin-9-yl)-2,2-dimethyl-3a,4,6,6a-tetrahydrofuro[3,4-d][1,3]dioxole-6-carboxylic acid (6.7 g, 7.9 mmol, 54% yield) as a brown solid, estimated to be ˜40% purity based on TLC

N-ethyl-N-isopropyl-propan-2-amine (2.74 mL, 15.73 mmol) was added to a solution of (3aR,4R,6S,6aS)-4-(6-chloropurin-9-yl)-2,2-dimethyl-3a,4,6,6a-tetrahydrofuro[3,4-d][1,3]dioxole-6-carboxylic acid (2.68 g, 7.87 mmol) and 1-PROPANEPHOSPHONIC ACID CYCLIC ANHYDRIDE (8 .mL, 15.73 mmol) in Ethyl acetate (30 mL) at 0 C. Note that the patent procedure missed the base. The reaction doesn't proceed without base, typically TEA or hunig base. The resolution solution was stirred at RT for 1 hr. TLC showed the completion of the reaction (9:1 DCM/MeOH).

The reaction was poured into ice-cold water, the aq. layer was extracted with EtOAc 3×. Note that the product was difficult to extract under strong acidic conditions. The combined organic layers were washed with sat. aq. NaHCO3, water, brine, dried over sodium sulfate, filtered and concentrated. The crude product was purified on a 40 g column to give (3aR,4R,6S,6aS)-4-(6-chloropurin-9-yl)-N-methoxy-N,2,2-trimethyl-3a,4,6,6a-tetrahydrofuro[3,4-d][1,3]dioxole-6-carboxamide (92a) (1.72 g, 4.48 mmol, 57.0% yield) as a white foamy solid.

The impure portion of acid was converted to the Weinreb amide following the same procedure as shown above to give another 3.6 g of the product, which made the total yield of these two steps over 80%.

### b) Synthesis of [(3aR,4R,6S,6aS)-4-(6-chloropurin-9-yl)-2,2-dimethyl-3a,4,6,6a-tetrahydrofuro[3,4-d][1,3]dioxol-6-yl]-(3,4-dichlorophenyl)methanone (92b)

To an ice-cold solution of (3aR,4R,6S,6aS)-4-(6-chloropurin-9-yl)-N-methoxy-N,2,2-trimethyl-3a,4,6,6a-tetrahydrofuro[3,4-d][1,3]dioxole-6-carboxamide (92a) (1.72 g, 4.48 mmol) in THE (30 mL) was added a solution of (3,4-Dichlorophenyl)magnesium bromide, 0.50 μM in 2-MeTHF (17.93 mL, 8.96 mmol) dropwise. The resulting mixture was warmed to rt, stirred for 2 hr. LCMS showed the completion of the reaction. The reaction mixture was poured onto ice-cold sat. aq. NH₄Cl solution, which was extracted with EtOAc. The organic layer was washed with brine, dried over Na₂SO₄, filtered and concentrated. The crude product was purified on a 20 g column, which was eluted with 0-50% EA/hexane to give [(3aR,4R,6S,6aS)-4-(6-chloropurin-9-yl)-2,2-dimethyl-3a,4,6,6a-tetrahydrofuro[3,4-d][1,3]dioxol-6-yl]-(3,4-dichlorophenyl)methanone (92b) (1.82 g, 3.87 mmol, 86.5% yield) as a light yellow solid. LCMS (M+H+) 469/471/473

### c) Synthesis of (1R)-1-[(3aR,4R,6S,6aR)-4-(6-chloropurin-9-yl)-2,2-dimethyl-3a,4,6,6a-tetrahydrofuro[3,4-d][1,3]dioxol-6-yl]-1-(3,4-dichlorophenyl)ethanol (92c)

To an ice-cold solution of [(3aR,4R,6S,6aS)-4-(6-chloropurin-9-yl)-2,2-dimethyl-3a,4,6,6a-tetrahydrofuro[3,4-d][1,3]dioxol-6-yl]-(3,4-dichlorophenyl)methanone (92b) (1.82 g, 3.87 mmol) in THE (30 mL) was added a solution of bromo(methyl)magnesium (3 M, 2.42 mL, 7.75 mmol) dropwise. The resulting mixture was warmed to rt, stirred for 2 hr. LCMS showed the completion of the reaction. The reaction mixture was poured onto ice-cold sat. aq. NH₄Cl solution, which was extracted with EtOAc. The organic layer was washed with brine, dried over Na₂SO₄, filtered and concentrated. The crude product was purified on a 20 g column, which was eluted with 0-50% EA/hexane to give (1R)-1-[(3aR,4R,6S,6aR)-4-(6-chloropurin-9-yl)-2,2-dimethyl-3a,4,6,6a-tetrahydrofuro[3,4-d][1,3]dioxol-6-yl]-1-(3,4-dichlorophenyl)ethanol (92c) (1.5 g, 3.1 mmol, 80% yield) as a light yellow solid and (1S)-1-[(3aR,4R,6S,6aR)-4-(6-chloropurin-9-yl)-2,2-dimethyl-3a,4,6,6a-tetrahydrofuro[3,4-d][1,3]dioxol-6-yl]-1-(3,4-dichlorophenyl)ethanol (171 mg, 0.352 mmol, 9% yield) as a yellow foamy solid. LCMS (M+H+) 475/477/479.

### d) Synthesis of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol hydrochloride (Ex. 92)

A mixture of N-ethyl-N-isopropyl-propan-2-amine (0.14 mL, 0.8200 mmol), O-Methylhydroxylamine hydrochloride (72.39 mg, 0.8200 mmol), (1R)-1-[(3aR,4R,6S,6aR)-4-(6-chloropurin-9-yl)-2,2-dimethyl-3a,4,6,6a-tetrahydrofuro[3,4-d][1,3]dioxol-6-yl]-1-(3,4-dichlorophenyl)ethanol (92c) (200 .mg, 0.4100 mmol) in IPA (5 mL) was purged with N2, sealed and irradiated with MW at 120 C for 20 min. LCMS showed the consumption of the st.m. The reaction mixture was diluted with DCM and water. The aq. layer was extracted with DCM several times until not floating yellow solids visible. All organic layers were combined, dried over Na2SO4, filtered and concentrated to give crude (1R)-1-[(3aR,4R,6S,6aR)-4-[(6Z)-6-methoxyimino-1H-purin-9-yl]-2,2-dimethyl-3a,4,6,6a-tetrahydrofuro[3,4-d][1,3]dioxol-6-yl]-1-(3,4-dichlorophenyl)ethanol (200 mg, 0.4029 mmol, 98% yield) as a light yellow solid

A mixture of (1R)-1-[(3aR,4R,6S,6aR)-4-[(6Z)-6-methoxyimino-1H-purin-9-yl]-2,2-dimethyl-3a,4,6,6a-tetrahydrofuro[3,4-d][1,3]dioxol-6-yl]-1-(3,4-dichlorophenyl)ethanol (200 .mg, 0.4000 mmol) and HCl (0.16 mL, 2.16 mmol) in Methanol (2 mL) (pre-mixed) was stirred at RT overnight. White precipitate was formed, filtered and dried to give (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol hydrochloride (Ex. 92) (148 mg, 0.29 mmol, 72% yield). HPLC 97% pure; LCMS 456.0/458.0; ¹HNMR (400 MHz, CD₃OD) δ 7.75 (d, J=2.0 Hz, 1H), 7.53-7.45 (m, 2H), 6.05 (d, J=6.8 Hz), 4.67 (dd, J=6.8, 5.2 Hz, 1H), 4.32 (d, J=2.0 Hz, 1H), 4.00 (m, 1H), 3.98 (s, 3H), 1.58 (s, 3H).

### Example 92A. (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol, free base crystals (92A)

9.5 g of (2S,3S,4R,5R)-2-[(1R)-1-(3,4-dichlorophenyl)-1-hydroxy-ethyl]-5-[(6Z)-6-methoxyimino-1H-purin-9-yl]tetrahydrofuran-3,4-diol, white solid, with 99.0% purity was suspended in deionized water (30.0 mL) and stirred at 100° C. for 2 h, cooled, filtered, dried in vacuum to give 92A (8.3 g) as a white crystalline solid, m.p. 238.5° C.

### Example 93. (2S,3S,4R,5R)-2-[(1R)-Example 931-(3,4-dichlorophenyl)-1-hydroxy-ethyl]-5-[(6Z)-6-ethoxyimino-1H-purin-9-yl]tetrahydrofuran-3,4-diol (93)

Example 93 was prepared following the similar procedures as Ex. 92 except for substituting O-Methylhydroxylamine hydrochloride with O-Ethylhydroxylamine hydrochloride. ¹H NMR (400 MHz, DMSO-d₆+D₂O) δ 8.45 (s, 1H), 8.09 (s, 1H), 7.77-7.78 (m, 1H), 7.61-7.63 (m, 1H), 7.51-7.54 (m, 1H), 5.88-5.90 (m, 1H), 4.47-4.50 (m, 1H), 4.18-4.19 (m, 1H), 4.04-4.09 (q, 2H), 3.72-3.73 (m, 1H), 1.45 (s, 3H), 1.27-1.30 (t, 3H).

### Example 94. (2S,3S,4R,5R)-2-[(1R)-1-(4-chloro-3-methylphenyl)-1-hydroxy-ethyl]-5-[(6Z)-6-methoxyimino-1H-purin-9-yl]tetrahydrofuran-3,4-diol (94)

Example 94 (HCl salt) was prepared following the similar procedures as Ex. 92 except for substituting 3,4-dichlorophenylmagnesium bromide with 4-chloro-3-methylphenylmagnesium bromide. ¹H NMR (600 MHz, Methanol-d4) δ 8.76 (s, 1H), 8.31 (s, 1H), 7.48 (d, J=2.1 Hz, 1H), 7.39-7.29 (m, 2H), 6.10 (d, J=6.9 Hz, 1H), 4.68 (dd, J=5.2, 6.9 Hz, 1H), 4.34 (d, J=1.9 Hz, 1H), 4.03 (dd, J=1.9, 5.2 Hz, 1H), 4.01 (s, 3H), 2.40 (s, 3H), 1.58 (s, 3H).

### Example 95. (2S,3S,4R,5R)-2-[(1R)-1-(4-chloro-3-methylphenyl)-1-hydroxy-ethyl]-5-[(6Z)-6-ethoxyimino-1H-purin-9-yl]tetrahydrofuran-3,4-diol (95)

Example 95 (HCl salt) was prepared following the similar procedures as Ex. 94 except for substituting O-Methylhydroxylamine hydrochloride with O-Ethylhydroxylamine hydrochloride. ¹H NMR (600 MHz, Methanol-d₄) δ 8.76 (s, 1H), 8.36 (s, 1H), 7.49 (d, J=2.1 Hz, 1H), 7.40-7.32 (m, 2H), 6.12 (d, J=6.9 Hz, 1H), 4.68 (dd, J=5.2, 6.9 Hz, 1H), 4.34 (d, J=1.9 Hz, 1H), 4.25 (q, J=7.0 Hz, 2H), 4.03 (dd, J=1.9, 5.2 Hz, 1H), 2.40 (s, 3H), 1.58 (s, 3H), 1.45 (t, J=7.0 Hz, 3H).

### Example 96. 7-((2R,3R,4S,5S)-5-((R)-1-(4-chloro-3-fluorophenyl)-1-hydroxyethyl)-3,4-dihydroxytetrahydrofuran-2-yl)-1,5,6,7-tetrahydro-4H-pyrrolo[2,3-d]pyrimidin-4-one O-methyl oxime (96)

Example 96 (TFA salt) was prepared similarly to that of Ex. 60. ¹H NMR (400 MHz, Methanol-d4) δ 8.24 (s, 1H), 7.68 (d, J=3.7 Hz, 1H), 7.51-7.43 (m, 2H), 7.35 (dd, J=2.0, 8.5 Hz, 1H), 6.73 (d, J=3.6 Hz, 1H), 6.16 (d, J=7.5 Hz, 1H), 4.60 (dd, J=5.3, 7.5 Hz, 1H), 4.24 (d, J=1.5 Hz, 1H), 4.00-3.90 (m, 4H), 1.55 (s, 3H).

### Example 97. 7-((2R,3R,4S,5R)-5-((R)-(4-chlorophenyl)(hydroxy)methyl)-3,4-dihydroxytetrahydrofuran-2-yl)-1,7-dihydro-4H-pyrrolo[2,3-d]pyrimidin-4-one O-ethyl oxime (97)

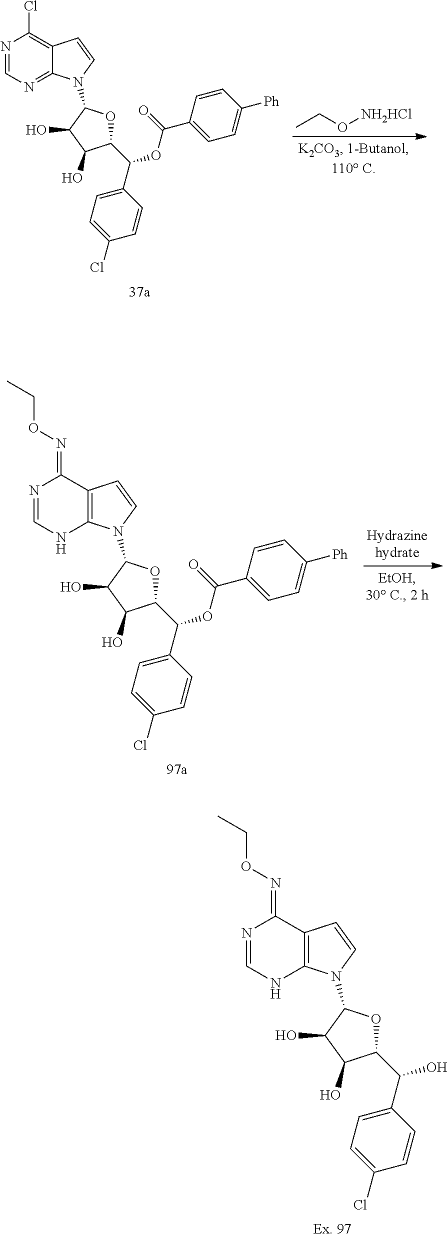

### a) Synthesis of [(R)-(4-chlorophenyl)-[(2S,3S,4R,5R)-5-[(4Z)-4-ethoxyimino-1H-pyrrolo[2,3-d]pyrimidin-7-yl]-3,4-dihydroxy-tetrahydrofuran-2-yl]methyl] 4-phenylbenzoate (97a)

To a solution of [(R)-(4-chlorophenyl)-[(2S,3S,4R,5R)-5-(4-chloropyrrolo[2,3-d]pyrimidin-7-yl)-3,4-dihydroxy-tetrahydrofuran-2-yl]methyl] 4-phenylbenzoate (37a) (390.0 mg, 0.68 mmol) in 1-Butanol (20.0 mL) was added Ethoxyamine hydrochloride (330.0 mg, 3.38 mmol) and Triethylamine (0.8 mL, 5.41 mmol). The reaction mixture was stirred at 110° C. for 120 h. The reaction mixture was concentrated to give crude which was purified by silica gel column chromatography (EA:PE=5:1 to 1:1) to give [(R)-(4-chlorophenyl)-[(2S,3S,4R,5R)-5-[(4Z)-4-ethoxyimino-1H-pyrrolo[2,3-d] pyrimidin-7-yl]-3,4-dihydroxy-tetrahydrofuran-2-yl]methyl] 4-phenylbenzoate (97a) (40.0 mg, 0.07 mmol, 9.6% yield) as a yellow solid. LCMS [M+H]: 601.4.

### b) Synthesis of (2R,3S,4R,5R)-2-[(R)-(4-chlorophenyl)-hydroxy-methyl]-5-[(4Z)-4-ethoxyimino-1H-pyrrolo[2,3-d]-pyrimidin-7-yl]tetrahydrofuran-3,4-diol hydrochloride (97)

To a mixture of Hydrazine hydrate (1.0 mL, 20.58 mmol) and Ethanol (1.0 mL) was added [(R)-(4-chlorophenyl)-[(2S,3S,4R,5R)-5-[(4Z)-4-ethoxyimino-1H-pyrrolo[2,3-d]pyrimidin-7-yl]-3,4-dihydroxy-tetrahydrofuran-2-yl]methyl] 4-phenylbenzoate (97a) (40.0 mg, 0.07 mmol). The reaction mixture was stirred at 30° C. for 2 h. The mixture reaction was concentrated to give crude product which was purified by prep-HPLC, eluted with CH₃CN in H₂O (0.1% TFA) from 10.0% to 95.0% to obtain (2R,3S,4R,5R)-2-[(R)-(4-chlorophenyl)-hydroxy-methyl]-5-[(4Z)-4-ethoxyimino-1H-pyrrolo[2,3-d]-pyrimidin-7-yl]tetrahydrofuran-3,4-diol hydrochloride (97) (15.9 mg, 0.03 mmol, 51.6% yield) as a white solid. LCMS [M+H]: 421.2. ¹H NMR (400 M Hz, DMSO-d₆): δ 8.05 (s, 1H), 7.57 (s, 1H), 7.35-7.42 (m, 4H), 6.71 (s, 1H), 6.03 (d, J=8.0 Hz, 1H), 4.77 (d, J=4.0 Hz, 1H), 4.49-4.52 (m, 1H), 4.09 (d, J=4 Hz, 1H), 4.01-4.07 (m, 2H), 3.97-3.99 (m, 1H), 1.28-1.32 (m, 3H). ¹H NMR (400 MHz, DMSO-d₆+D₂O): δ 8.20 (s, 1H), 7.62 (s, 1H), 7.36-7.42 (m, 4H), 6.74 (s, 1H), 6.06 (d, J=8.0 Hz, 1H), 4.77 (d, J=4.0 Hz, 1H), 4.49-4.52 (m, 1H), 4.10 (d, J=4 Hz, 1H), 4.04-4.07 (m, 2H), 4.00-4.01 (d, J=4.0 Hz, 1H), 1.29-1.33 (m, 3H).

### Example 98. 7-((2R,3R,4S,5R)-3,4-dihydroxy-5-((R)-hydroxy(3-methyl-4-(trifluoromethyl)phenyl)methyl)tetrahydrofuran-2-yl)-1,7-dihydro-4H-pyrrolo[2,3-d]pyrimidin-4-one O-methyl oxime (98)

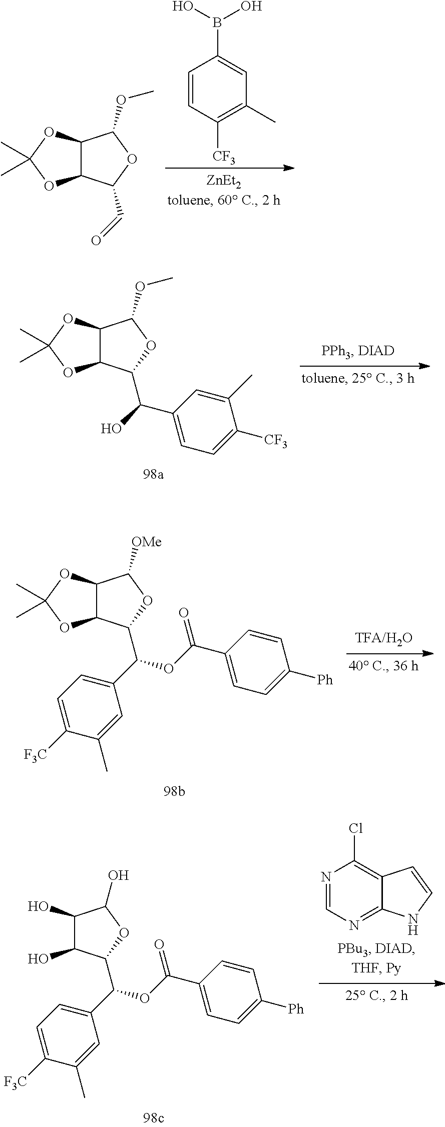

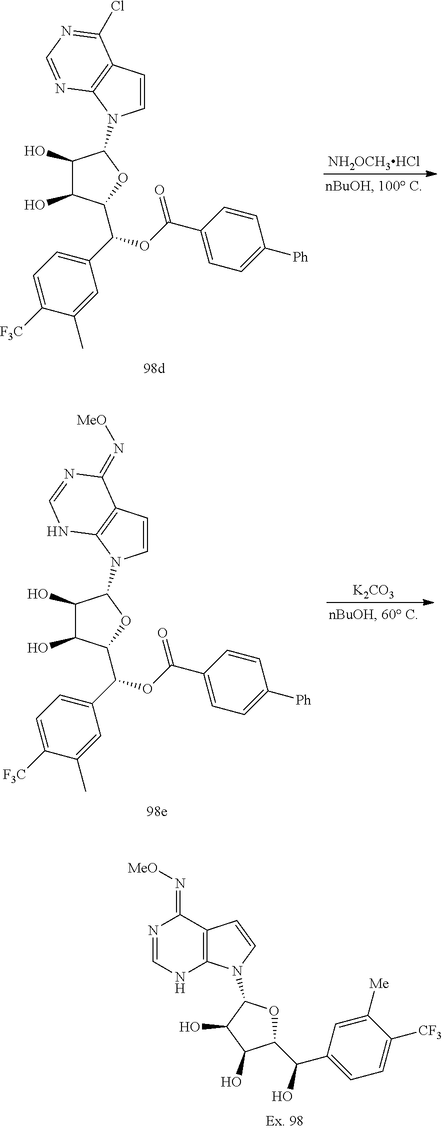 [SMILES: Cc1cc([C@@H](OC(=O)c2ccc(-c3ccccc3)cc2)[C@H]2O[C@@H](n3ccc4c(Cl)ncnc43)[C@H](O)[C@@H]2O)ccc1C(F)(F)F ; "NH2OCH3·HCl" ; CO/N=c1\nc[nH]c2c1ccn2[C@@H]1O[C@H]([C@H](OC(=O)c2ccc(-c3ccccc3)cc2)c2ccc(C(F)(F)F)c(C)c2)[C@@H](O)[C@H]1O ; "K2CO3" ; CO/N=c1\nc[nH]c2c1ccn2[C@@H]1O[C@H]([C@H](O)c2ccc(C(F)(F)F)c(C)c2)[C@@H](O)[C@H]1O]

### a) Synthesis of (S)-[(3aR,4R,6R,6aR)-4-methoxy-2,2-dimethyl-3a,4,6,6a-tetrahydrofuro[3,4-d][1,3]dioxol-6-yl]-[3-methyl-4-(trifluoromethyl)phenyl]methanol (98a)

To a solution of [3-methyl-4-(trifluoromethyl)phenyl]boronic acid (484.1 mg, 2.37 mmol) in Toluene (10.0 mL), Diethylzine (3.6 mL, 7.12 mmol) was added slowly at 25° C. The mixture was stirred at 60° C. for 1 h. (3aR,4R,6S,6aR)-4-methoxy-2,2-dimethyl-3a,4,6,6a-tetrahydrofuro[3,4-d] [1,3]dioxole-6-carbaldehyde (400.0 mg, 1.98 mmol) in Toluene (6.0 mL) was added slowly at 60° C. The mixture was stirred at 60° C. for 2 h. TLC (PE:EA=5:1) showed the reaction was completed. Water (4.0 mL) was added to quench the reaction. The mixture was filtered. The filtrate was concentrated and purified by reversed-phase combi-flash, eluted with CH₃CN in H₂O (neutral condition) from 5.0% to 85.0% to give (S)-[(3aR,4R,6R,6aR)-4-methoxy-2,2-dimethyl-3a,4,6,6a-tetrahydrofuro[3,4-d][1,3]dioxol-6-yl]-[3-methyl-4-(trifluoromethyl)phenyl]methanol (98a) (350.0 mg, 0.96 mmol, 48.6% yield) as a white solid. ¹H NMR (400 M Hz, DMSO-d₆): δ 7.64 (d, J=8.0 Hz, 1H), 7.45 (s, 1H), 7.39 (d, J=8.0 Hz, 1H), 5.38 (d, J=5.6 Hz, 1H), 4.96 (s, 1H), 4.48-4.56 (m, 3H), 4.19 (d, J=8.4 Hz, 1H), 3.32 (s, 3H), 2.45 (s, 3H), 1.34 (s, 3H), 1.17 (s, 3H). ¹⁹F NMR (376 μM Hz, DMSO-d₆): δ −59.99 (s, 3F).

### b) Synthesis of [(R)-[(3aR,4R,6R,6aR)-4-methoxy-2,2-dimethyl-3a,4,6,6a-tetrahydrofuro[3,4-d][1,3]dioxol-6-yl]-[3-methyl-4-(trifluoromethyl)phenyl]methyl]-4-phenylbenzoate (98b)

To a mixture of (S)-[(3aR,4R,6R,6aR)-4-methoxy-2,2-dimethyl-3a,4,6,6a-tetrahydrofuro[3,4-d][1,3] dioxol-6-yl]-[3-methyl-4-(trifluoromethyl)phenyl]methanol (98a) (3200.0 mg, 8.83 mmol), 4-phenylbenzoic acid (2625.9 mg, 13.25 mmol) and Triphenylphosphine (3474.6 mg, 13.25 mmol) in Toluene (50.0 mL) was added DIAD (2.6 mL, 13.25 mmol) at 0° C. The mixture was stirred at 25° C. for 3 h. The mixture was concentrated and purified by reversed-phase combi-flash, eluted with CH₃CN in H₂O (neutral) from 5.0% to 95.0% to give [(R)-[(3aR,4R,6R,6aR)-4-methoxy-2,2-dimethyl-3a,4,6,6a-tetrahydrofuro[3,4-d][1,3]dioxol-6-yl]-[3-methyl-4-(trifluoromethyl)phenyl]methyl]-4-phenylbenzoate (98b) (2700.0 mg, 4.98 mmol, 56.4% yield) as a white solid. ¹H NMR (400 M Hz, DMSO-d₆): δ 8.12 (d, J=8.0 Hz, 2H), 7.86 (d, J=8.0 Hz, 2H), 7.52 (d, J=8.0 Hz, 2H), 7.70 (d, J=8.0 Hz, 1H), 7.62 (s, 1H), 7.50-7.56 (m, 3H), 7.45 (d, J=7.6 Hz, 1H), 5.90 (d, J=8.4 Hz, 1H), 5.01 (d, J=6.0 Hz, 1H), 4.93 (s, 1H), 4.67-4.71 (m, 2H), 3.15 (s, 3H), 2.47 (s, 3H), 1.40 (s, 3H), 1.27 (s, 3H). ¹⁹F NMR (376 M Hz, DMSO-d6): δ −60.22 (s, 3F).

### c) Synthesis of [(R)-[3-methyl-4-(trifluoromethyl)phenyl]-[(2S,3S,4R,5R)-3,4,5-trihydroxytetrahydrofuran-2-yl]methyl] 4-phenylbenzoate (98c)

A mixture of [(R)-[(3aR,4R,6R,6aR)-4-methoxy-2,2-dimethyl-3a,4,6,6a-tetrahydrofuro[3,4-d][1,3] dioxol-6-yl]-[3-methyl-4-(trifluoromethyl)phenyl]methyl] 4-phenylbenzoate (98b) (2700.0 mg, 4.98 mmol) in water (30.0 mL, 1664.8 mmol) and TFA (30.0 mL, 405.19 mmol) was heated to 40° C. and stirred for 36 h. LCMS showed the reaction was completed. The mixture was concentrated and purified by reversed-phase combi-flash, eluted with CH₃CN in H₂O (neutral) from 5.0% to 95.0% to give [(R)-[3-methyl-4-(trifluoromethyl)phenyl]-[(2S,3S,4R,5R)-3,4,5-trihydroxytetrahydrofuran-2-yl]methyl] 4-phenylbenzoate (98c) (1600.0 mg, 3.28 mmol, 65.8% yield) as white solid. LCMS [M−H]: 487.1.

### d) Synthesis of [(R)-[(2S,3S,4R,5R)-5-(4-chloropyrrolo[2,3-d]pyrimidin-7-yl)-3,4-dihydroxy-tetrahydrofuran-2-yl]-[3-methyl-4-(trifluoromethyl)phenyl]methyl] 4-phenylbenzoate (98d)

To a solution of 4-Chloro-7H-pyrrolo[2,3-d]pyrimidine (380.4 mg, 2.48 mmol) in dry THE (60.0 mL) was added Pyridine (0.2 mL, 2.48 mmol), Tributylphosphane (1.2 mL, 4.95 mmol) and DIAD (1.1 mL, 5.45 mmol) at 25° C. [(R)-[3-methyl-4-(trifluoromethyl)phenyl]-[(2S,3S,4R,5R)-3,4,5-trihydroxytetrahydrofuran-2-yl]methyl] 4-phenylbenzoate (98c) (1210.0 mg, 2.48 mmol)) in dry THE (20.0 mL) was added at once. The reaction mixture was stirred at 25° C. for 2 h. LCMS showed the reaction was completed. The reaction mixture was purified by reversed-phase combi-flash, eluted with CH₃CN in H₂O (neutral condition) from 10.0% to 95.0% to afford [(R)-[(2S,3S,4R,5R)-5-(4-chloropyrrolo[2,3-d]pyrimidin-7-yl)-3,4-dihydroxy-tetrahydrofuran-2-yl]-[3-methyl-4-(trifluoromethyl)phenyl]methyl] 4-phenylbenzoate (98d) (1070.0 mg, 1.71 mmol, 69.2% yield) as a pale yellow solid. LCMS [M+H]: 624.3.

### e) Synthesis of [(R)-[(2S,3S,4R,5R)-3,4-dihydroxy-5-[(4Z)-4-methoxyimino-1H-pyrrolo[2,3-d]pyrimidin-7-yl]tetrahydrofuran-2-yl]-[3-methyl-4-(trifluoromethyl)phenyl]methyl] 4-phenylbenzoate (98e)

To a solution of [(R)-[(2S,3S,4R,5R)-5-(4-chloropyrrolo[2,3-d]pyrimidin-7-yl)-3,4-dihydroxy-tetra hydrofuran-2-yl]-[3-methyl-4-(trifluoromethyl)phenyl]methyl] 4-phenylbenzoate (98d) (520.0 mg, 0.83 mmol) in 1-Butanol (20.0 mL), Potassium carbonate (921.4 mg, 6.67 mmol) and Methoxy ammonium chloride (348.0 mg, 4.17 mmol) was added. The mixture was stirred at 100° C. for 6 h. LCMS showed the reaction was completed. The mixture was filtered and concentrated to give crude product which was used for the next step directly.

### f) Synthesis of (2R,3S,4R,5R)-2-[(R)-hydroxy-[3-methyl-4-(trifluoromethyl)phenyl]methyl]-5-[(4Z)-4-methoxyimino-1H-pyrrolo[2,3-d]pyrimidin-7-yl]tetrahydrofuran-3,4-diol hydrochloride (98)

To a solution of [(R)-[(2S,3S,4R,5R)-3,4-dihydroxy-5-[(4Z)-4-methoxyimino-1H-pyrrolo[2,3-d] pyrimidin-7-yl]tetrahydrofuran-2-yl]-[3-methyl-4-(trifluoromethyl)phenyl]methyl] 4-phenylbenzoate (98e) (520.0 mg, 0.60 mmol) in 1-Butanol (30.0 mL), Potassium carbonate (249.4 mg, 1.80 mmol) was added. The mixture was stirred at 60° C. for 2 h. LCMS showed the reaction was completed. The mixture was filtered and washed with EA (50.0 mL). The combined filtrate was concentrated and acidified with 1 M HCl to pH≈2 and purified by prep-HPLC, eluted with CH₃CN in H₂O (0.1% TFA) from 5.0% to 95.0% to give the solution of the desired product. 5 drops con. HCl was added to the solution and the solution was lyophilized to give (2R,3S,4R,5R)-2-[(R)-hydroxy-[3-methyl-4-(trifluoromethyl)phenyl]methyl]-5-[(4Z)-4-methoxyimino-1H-pyrrolo[2,3-d]pyrimidin-7-yl]tetrahydrofuran-3,4-diol hydrochloride (98) (79.4 mg, 0.16 mmol, 26.8% yield) as a yellow solid. ¹H NMR (400 M Hz, DMSO-d₆): δ 8.13 (s, 1H), 7.75-7.62 (m, 2H), 7.45 (s, 1H), 7.40 (d, J=8.0 Hz, 1H), 6.67 (s, 1H), 6.05 (d, J=7.2 Hz, 1H), 4.82 (d, J=5.2 Hz, 1H), 4.49-4.53 (m, 1H), 4.10 (d, J=4.8 Hz, 1H), 4.04 (d, J=4.8 Hz, 1H), 3.83 (s, 3H), 2.41 (s, 3H). ¹H NMR (400 M Hz, DMSO-d6+D₂O): δ 8.13 (s, 1H), 7.62 (d, J=7.6 Hz, 1H), 7.53 (d, J=3.6 Hz, 1H), 7.39-7.43 (m, 2H), 6.64 (d, J=3.6 Hz, 1H), 6.05 (d, J=7.6 Hz, 1H), 4.82 (d, J=4.8 Hz, 1H), 4.49-4.53 (m, 1H), 4.11 (d, J=5.2 Hz, 1H), 4.07 (d, J=4.8 Hz, 1H), 3.83 (s, 3H), 2.41 (s, 3H). ¹⁹F NMR (376 M Hz, DMSO-d₆): δ −59.93 (s, 3F).

### Example 99. 7-((2R,3R,4S,5S)-5-((R)-1-(3,4-dichlorophenyl)-1-hydroxyethyl)-3,4-dihydroxytetrahydrofuran-2-yl)-1,7-dihydro-4H-pyrrolo[2,3-d]pyrimidin-4-one O-ethyl oxime (99)

Example 99 was prepared similarly to that of Example 60. ¹H NMR (400 M Hz, DMSO-d₆): δ 8.14 (br s, 1H), 7.77 (d, J=1.2 Hz, 1H), 7.62-7.51 (m, 3H), 6.67 (br s, 1H), 6.01 (br s, 1H), 4.42 (br s, 1H), 4.10 (d, J=0.8 Hz, 1H), 4.04 (m, 2H), 3.71 (d, J=3.2 Hz, 1H), 1.44 (s, 3H), 1.30 (t, J=4.8 Hz, 3H).

### Example 100. 7-((2R,3R,4S,5R)-5-((R)-(3,4-dichlorophenyl)(hydroxy)methyl)-3,4-dihydroxytetrahydrofuran-2-yl)-5-(2-hydroxyethyl)-1,5-dihydro-4H-714-pyrrolo[2,3-d]pyrimidin-4-one O-methyl oxime (100)

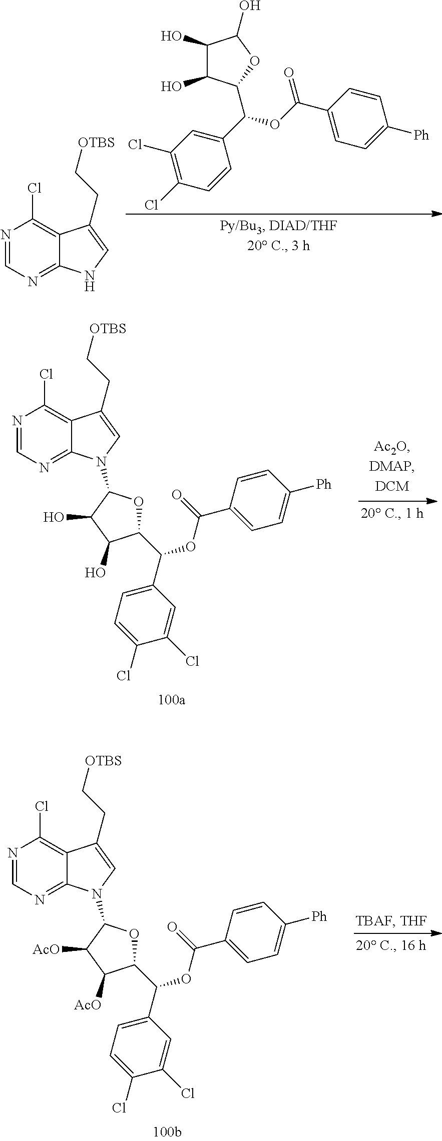

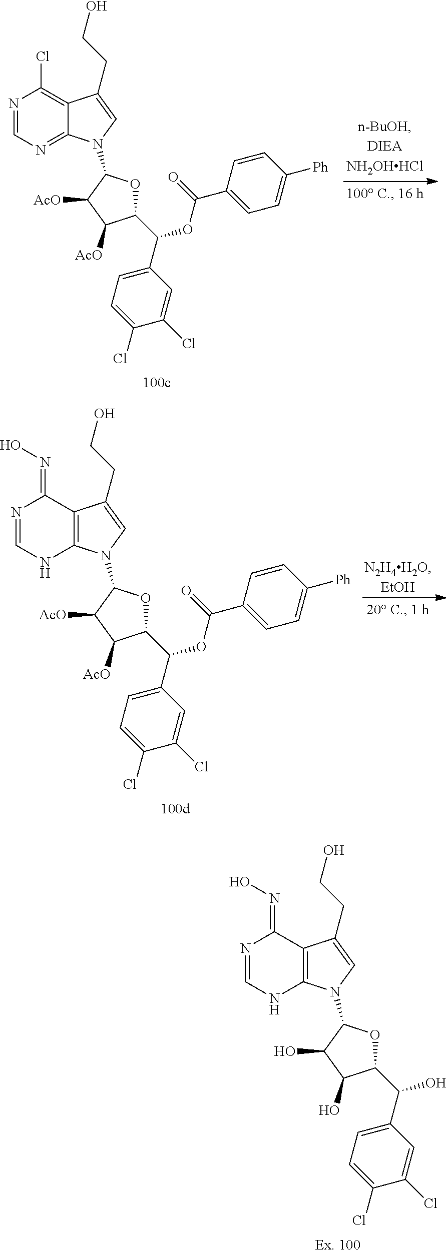 [SMILES: CC(=O)O[C@H]1[C@@H](OC(C)=O)[C@H](n2cc(CCO)c3c(Cl)ncnc32)O[C@@H]1[C@H](OC(=O)c1ccc(-c2ccccc2)cc1)c1ccc(Cl)c(Cl)c1 ; CC(=O)O[C@H]1[C@@H](OC(C)=O)[C@H](n2cc(CCO)c3/c(=N/O)nc[nH]c32)O[C@@H]1[C@H](OC(=O)c1ccc(-c2ccccc2)cc1)c1ccc(Cl)c(Cl)c1 ; OCCc1cn([C@@H]2O[C@H]([C@H](O)c3ccc(Cl)c(Cl)c3)[C@@H](O)[C@H]2O)c2[nH]cn/c(=N\O)c12]

### a) Synthesis of [(R)-[(2S,3S,4R,5R)-5-[5-[2-[tert-butyl(dimethyl)silyl]oxyethyl]-4-chloro-pyrrolo[2,3-d]pyrimidin-7-yl]-3,4-dihydroxy-tetrahydrofuran-2-yl]-(3,4-dichlorophenyl)methyl] 4-phenylbenzoate (100a)

To a solution of [(R)-(3,4-dichlorophenyl)-[(2S,3S,4R)-3,4,5-trihydroxytetrahydrofuran-2-yl]methyl] 4-phenylbenzoate (2.00 g, 4.21 mmol) in THE (35.0 mL) was added PBu₃ (2.1 mL, 8.42 mmol), pyridine (0.3 mL, 4.21 mmol), DIAD (1.6 mL, 8.42 mmol) and tert-butyl-[2-(4-chloro-7H-pyrrolo [2,3-d]pyrimidin-5-yl)ethoxy]-dimethyl-silane (1.31 g, 4.21 mmol) under N₂. The mixture was stirred at 20° C. for 3 h. The solvent was removed in vacuum to give crude product which was purified by silica chromatography (PE:EA=10:1 to 3:1 to give the [(R)-[(2S,3S,4R,5R)-5-[5-[2-[tert-butyl(dimethyl)silyl]oxyethyl]-4-chloro-pyrrolo[2,3-d]pyrimidin-7-yl]-3,4-dihydroxy-tetrahydrofuran-2-yl]-(3,4-dichlorophenyl)methyl] 4-phenylbenzoate (100a) (1.20 g, 1.56 mmol, 37.1% yield) as a yellow solid. LCMS [M+H]: 768.2.

### b) Synthesis of [(R)-[(2R,3R,4R,5R)-3,4-diacetoxy-5-[5-[2-[tert-butyl(dimethyl)silyl]oxyethyl]-4-chloro-pyrrolo[2,3-d]pyrimidin-7-yl]tetrahydrofuran-2-yl]-(3,4-dichlorophenyl)methyl] 4-phenylbenzoate (100b)

To a solution of [(R)-[(2S,3S,4R,5R)-5-[5-[2-[tert-butyl(dimethyl)silyl]oxyethyl]-4-chloro-pyrrolo [2,3-d]pyrimidin-7-yl]-3,4-dihydroxy-tetrahydrofuran-2-yl]-(3,4-dichlorophenyl)methyl]4-phenylbenzoate (100a) (1.20 g, 1.56 mmol) in DCM (15.0 mL) was added Pyridine (0.6 mL, 7.80 mmol), DMAP (19.0 mg, 0.16 mmol) and Ac₂O (0.80 g, 7.80 mmol). The mixture was stirred at 20° C. for 1 h. The solvent was removed in vacuum and EA (100.0 mL) was added. The mixture was washed with NH₄Cl solution (50.0 mL), brine (50.0 mL), dried over Na₂SO₄, concentrated in vacuum to give [(R)-[(2R,3R,4R,5R)-3,4-diacetoxy-5-[5-[2-[tert-butyl(dimethyl)silyl]oxyethyl]-4-chloro-pyrrolo[2,3-d]pyrimidin-7-yl]tetrahydrofuran-2-yl]-(3,4-dichlorophenyl)methyl] 4-phenylbenzoate (100b) (1.20 g, 1.40 mmol, 90.1% yield) as a white solid. LCMS [M+H]: 852.2.

### c) Synthesis of [(R)-[(2R,3R,4R,5R)-3,4-diacetoxy-5-[4-chloro-5-(2-hydroxyethyl)pyrrolo[2,3-d]pyrimidin-7-yl]tetrahydrofuran-2-yl]-(3,4-dichlorophenyl)methyl] 4-phenylbenzoate (100c)

To a solution of [(R)-[(2R,3R,4R,5R)-3,4-diacetoxy-5-[5-[2-[tert-butyl(dimethyl)silyl]oxyethyl]-4-chloro-pyrrolo[2,3-d]pyrimidin-7-yl]tetrahydrofuran-2-yl]-(3,4-dichlorophenyl)methyl]4-phenylbenzoate (100b) (1.20 g, 1.41 mmol) in THE (10.0 mL) was added TBAF (0.5 mL, 4.0 M, 2.11 mmol). The mixture was stirred at 20° C. for 16 h. The solvent was removed in vacuum and the residue was purified by silica chromatography (PE:EA=10:1 to 2:1 to give [(R)-[(2R,3R,4R,5R)-3,4-diacetoxy-5-[4-chloro-5-(2-hydroxyethyl)pyrrolo[2,3-d]pyrimidin-7-yl]tetrahydrofuran-2-yl]-(3,4-dichlorophenyl)methyl] 4-phenylbenzoate (100c) (0.80 g, 1.08 mmol, 76.9% yield) as a gray solid. LCMS [M+H]: 738.1.

### d) Synthesis of [(R)-(3,4-dichlorophenyl)-[(2S,3S,4R,5R)-3,4-dihydroxy-5-[(4Z)-5-(2-hydroxyethyl)-4-methoxyimino-1H-pyrrolo[2,3-d]pyrimidin-7-yl]tetrahydrofuran-2-yl]methyl] 4-phenylbenzoate (100d)

To a solution of [(R)-[(2R,3R,4R,5R)-3,4-diacetoxy-5-[4-chloro-5-(2-hydroxyethyl)pyrrolo[2,3-d]-pyrimidin-7-yl]tetrahydrofuran-2-yl]-(3,4-dichlorophenyl)methyl] 4-phenylbenzoate (100c) (0.30 g, 0.41 mmol) in 1-Butanol (6.0 mL) was added DIEA (1.31 g, 10.15 mmol) and O-Methylhydroxylamine hydrochloride (0.68 g, 8.12 mmol). The mixture was stirred at 100° C. for 16 h. The solvent was removed in vacuum to give crude product which was purified by prep-HPLC, eluted with MeCN in H₂O (0.1% TFA) from 5.0% to 80.0%1 to give [(R)-(3,4-dichlorophenyl)-[(2S,3S,4R,5R)-3,4-dihydroxy-5-[(4Z)-5-(2-hydroxyethyl)-4-methoxyimino-1H-pyrrolo[2,3-d]pyrimidin-7-yl]tetrahydrofuran-2-yl]methyl] 4-phenylbenzoate (100d) (40.0 mg, 0.06 mmol, 14.8% yield) as a white solid. LCMS [M+H]: 735.1.

### e) Synthesis of (2R,3S,4R,5R)-2-[(R)-(3,4-dichlorophenyl)-hydroxy-methyl]-5-[(4Z)-5-(2-hydroxyethyl)-4-methoxyimino-1H-pyrrolo[2,3-d]pyrimidin-7-yl]tetrahydrofuran-3,4-diol (100)

To a solution of [(R)-(3,4-dichlorophenyl)-[(2S,3S,4R,5R)-3,4-dihydroxy-5-[(4Z)-5-(2-hydroxyethyl)-4-methoxyimino-1H-pyrrolo[2,3-d]pyrimidin-7-yl]tetrahydrofuran-2-yl]methyl]4-phenylbenzoate (100d) (40.0 mg, 0.06 mmol) in Ethanol (2.0 mL) was added N₂H₄.H₂O (15.0 mg, 0.30 mmol). The mixture was stirred at 20° C. for 1 h. The solvent was removed in vacuum to give crude product which was purified by prep-HPLC, eluted with MeCN in H₂O (0.1% TFA) from 10.0% to 70.0% to afford (2R,3S,4R,5R)-2-[(R)-(3,4-dichlorophenyl)-hydroxy-methyl]-5-[(4Z)-5-(2-hydroxyethyl)-4-methoxyimino-1H-pyrrolo[2,3-d]pyrimidin-7-yl]tetrahydrofuran-3,4-diol (Ex. 100) (2.5 mg, 0.005 mmol, 8.4% yield) as a white solid. LCMS [M+H]: 485.3. ¹H NMR (400 M Hz, DMSO-d₆): δ 10.87 (s, 1H), 7.59-7.55 (m, 2H), 7.43 (s, 1H), 7.37 (d, J=9.0 Hz, 1H), 6.90 (s, 1H), 6.11 (d, J=4.2 Hz, 1H), 5.81 (d, J=7.5 Hz, 1H), 5.19 (d, J=6.8 Hz, 1H), 5.06 (d, J=3.8 Hz, 1H), 4.75-4.73 (m, 1H), 4.60-4.57 (m, 1H), 4.37-4.36 (m, 1H), 4.02 (brs, 1H), 3.91-3.90 (m, 1H), 3.71 (s, 3H), 3.64-3.59 (m, 2H), 2.75 (t, J=7.0 Hz, 2H).

### Example 101. 7-((2R,3R,4S,5R)-5-((R)-(4-chlorophenyl)(hydroxy)methyl)-3,4-dihydroxytetrahydrofuran-2-yl)-5-ethynyl-1,7-dihydro-4H-pyrrolo[2,3-d]pyrimidin-4-one O-methyl oxime (101)

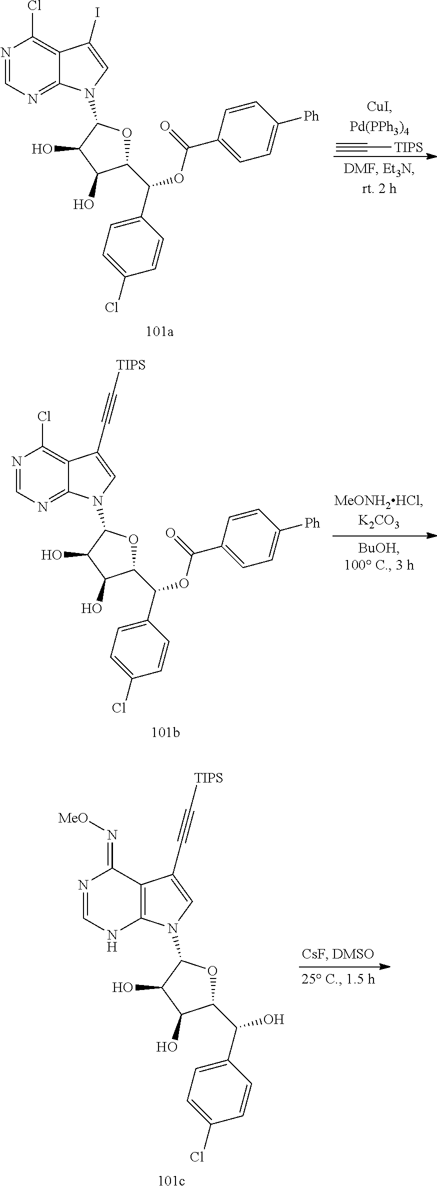

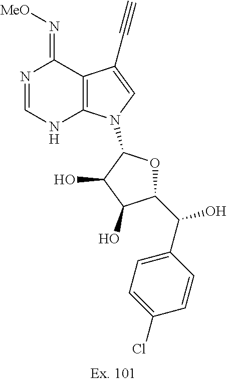 [SMILES: C#Cc1cn([C@@H]2O[C@H]([C@H](O)c3ccc(Cl)cc3)[C@@H](O)[C@H]2O)c2[nH]cn/c(=N\OC)c12]

### a) Preparation of [(R)-[(2S,3S,4R,5R)-5-(4-chloro-5-iodo-pyrrolo[2,3-d]pyrimidin-7-yl)-3,4-dihydroxy-tetrahydrofuran-2-yl]-(4-chlorophenyl)methyl] 4-phenylbenzoate (101a)

Compound 101a was prepared similarly to that of Int-2 except for substituting 6-chloropurine with 4-chloro-5-iodo-7H-pyrrolo[2,3-d]pyrimidine.

### b) Synthesis of [(R)-(4-chlorophenyl)-[(2S,3S,4R,5R)-5-[4-chloro-5-(2-triisopropylsilylethynyl)pyrrolo[2,3-d]pyrimidin-7-yl]-3,4-dihydroxy-tetrahydrofuran-2-yl]methyl] 4-phenylbenzoate (101b)

To a solution of [(R)-[(2S,3 S,4R,5R)-5-(4-chloro-5-iodo-pyrrolo[2,3-d]pyrimidin-7-yl)-3,4-dihydroxy-tetrahydrofuran-2-yl]-(4-chlorophenyl)methyl] 4-phenylbenzoate (101a) (3.00 g, 3.42 mmol), Pd(PPh₃)₄ (197.4 mg, 0.17 mmol) and CuI (65.1 mg, 0.34 mmol) in DMF (30.00 mL) was added (Triisopropylsilyl)acetylene (1.15 mL, 5.13 mmol) and TEA (1.42 mL, 10.25 mmol) under N₂. The reaction mixture was stirred at 25° C. for 18 h under N₂. LCMS showed the reaction was completed. The solution was filtered and the filtrate was poured into water, extracted with EA (100.0 mL×3). The organic layers were washed with brine (50.0 mL×3), dried over anhydrous Na₂SO₄, filtered and concentrated in vacuum to give crude product which was purified by silica gel column chromatography (PE:EA=10:1 to PE:EA=2:1) to give [(R)-(4-chlorophenyl)-[(2S,3S,4R,5R)-5-[4-chloro-5-(2-triisopropylsilylethynyl)pyrrolo[2,3-d]pyrimidin-7-yl]-3,4-dihydroxy-tetrahydrofuran-2-yl]methyl]4-phenylbenzoate (101b) (1.94 g, 2.31 mmol, 67.5% yield) as a white solid. LCMS [M+H]: 756.4.

### c) Synthesis of (2R,3S,4R,5R)-2-[(R)-(4-chlorophenyl)-hydroxy-methyl]-5-[(4Z)-4-methoxyimino-5-(2-tri-isopropyl silylethynyl)-1H-pyrrolo[2,3-d]pyrimidin-7-yl]tetrahydrofuran-3,4-diol (101c)

To a solution of [(R)-(4-chlorophenyl)-[(2S,3S,4R,5R)-5-[4-chloro-5-(2-triisopropylsilylethynyl) pyrrolo[2,3-d]pyrimidin-7-yl]-3,4-dihydroxy-tetrahydrofuran-2-yl]methyl]4-phenylbenzoate (101b) (0.90 g, 0.95 mmol) in 1-Butanol (10.00 mL) was added Methoxyammonium chloride (0.40 g, 4.76 mmol) and K₂CO₃ (1.18 g, 8.56 mmol). The reaction mixture was stirred at 100° C. for 3 h. LCMS showed the reaction was completed. The reaction mixture was adjusted to pH=7.0 and purified by reversed-phase combi-flash (neutral condition), eluted with MeCN in H₂O from 10.0% to 95.0% to give (2R,3S,4R,5R)-2-[(R)-(4-chlorophenyl)-hydroxy-methyl]-5-[(4Z)-4-methoxyimino-5-(2-tri-isopropyl silylethynyl)-1H-pyrrolo[2,3-d]pyrimidin-7-yl]tetrahydrofuran-3,4-diol (101c) (214.0 mg, 0.33 mmol, 34.5% yield) as a white solid. LCMS [M+H]: 587.4.

### d) Synthesis of (2R,3S,4R,5R)-2-[(R)-(4-chlorophenyl)-hydroxy-methyl]-5-[(4Z)-5-ethynyl-4-methoxyimino-1H-pyrrolo[2,3-d]pyrimidin-7-yl]tetrahydrofuran-3,4-diol (101)

To a solution of (2R,3S,4R,5R)-2-[(R)-(4-chlorophenyl)-hydroxy-methyl]-5-[(4Z)-4-methoxyimino-5-(2-triisopropylsilylethynyl)-1H-pyrrolo[2,3-d]pyrimidin-7-yl]tetrahydrofuran-3,4-diol (101c) (214.0 mg, 0.36 mmol) in DMSO (5.00 mL) and Methanol (0.10 mL) was added CsF (54.3 mg, 0.36 mmol) under N₂. The reaction mixture was stirred at 25° C. for 1.5 h. LCMS showed the reaction was completed. The reaction mixture was filtered and the filtrate was purified by prep-HPLC, eluted with MeCN in H₂O (0.1% NH₃.H₂O) from 10.0% to 95.0% to give crude product (95.0 mg, HPLC: 95.6%), the crude product was purified by prep-TLC (DCM:CH₃OH=10:1) to give 105.0 mg of crude product and further purified by reversed-phase combi-flash, eluted with MeCN in H₂O (neutral condition) from 10.0% to 95.0% to give crude product (95.0 mg, HPLC: 95.6%) to give (2R,3 S,4R,5R)-2-[(R)-(4-chlorophenyl)-hydroxy-methyl]-5-[(4Z)-5-ethynyl-4-methoxyimino-1H-pyrrolo[2,3-d]pyrimidin-7-yl]tetrahydrofuran-3,4-diol (Ex. 101) (55.0 mg, 0.13 mmol, 35.7% yield) as a white solid. LCMS [M+H]: 431.3. ¹H NMR (400 MHz, DMSO-d₆) δ 11.02 (d, J=3.2 Hz, 1H), 7.50 (s, 1H), 7.47 (d, J=3.6 Hz, 1H), 7.36-7.42 (m, 4H), 6.00 (d, J=4.4 Hz, 1H), 5.85 (d, J=7.6 Hz, 1H), 5.25 (d, J=6.8 Hz, 1H), 5.01 (d, J=4.4 Hz, 1H), 4.76 (t, J=4.4 Hz, 1H), 4.37-4.42 (m, 1H), 4.02 (t, J=4.0 Hz, 1H), 3.94 (d, J=4.4 Hz, 1H), 3.90 (s, 1H), 3.73 (s, 3H). ¹H NMR (400 MHz, DMSO-d₆+D₂O) δ 7.51 (s, 1H), 7.46 (s, 1H), 7.37-7.42 (m, 4H), 5.85 (d, J=7.6 Hz, 1H), 4.76 (d, J=4.4 Hz, 1H), 4.38-4.41 (m, 1H), 4.03 (d, J=4.8 Hz, 1H), 3.97 (d, J=4.8 Hz, 1H), 3.87 (s, 1H), 3.74 (s, 3H).

### Example 102. 7-((2R,3R,4S,5S)-5-((R)-(4-chloro-3-fluorophenyl)(methoxy)methyl)-3,4-dihydroxytetrahydrofuran-2-yl)-1,7-dihydro-4H-pyrrolo[2,3-d]pyrimidin-4-one O-methyl oxime (102)

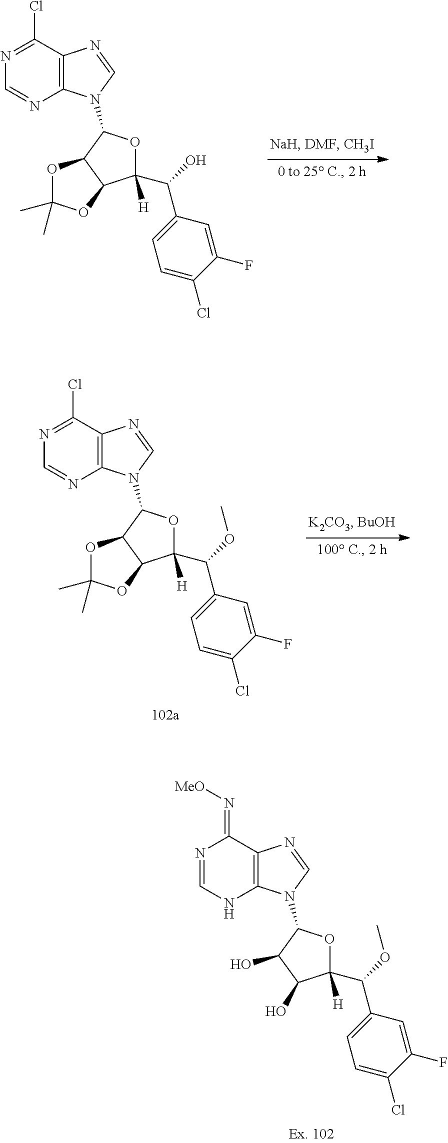 [SMILES: CC1(C)O[C@H]2[C@@H](O1)[C@H](n1cnc3c(Cl)ncnc31)O[C@@H]2[C@H](O)c1ccc(Cl)c(F)c1 ; CO[C@H](c1ccc(Cl)c(F)c1)[C@H]1O[C@@H](n2cnc3c(Cl)ncnc32)[C@@H]2OC(C)(C)O[C@@H]21 ; CO/N=c1\nc[nH]c2c1ncn2[C@@H]1O[C@H]([C@H](OC)c2ccc(Cl)c(F)c2)[C@@H](O)[C@H]1O]

### a) Synthesis of 7-[(3aR,4R,6R,6aR)-6-[(4-chloro-3-fluoro-phenyl)-methoxy-methyl]-2,2-dimethyl-3a,4,6,6a-tetrahydrofuro[3,4-d][1,3]dioxol-4-yl]-4-chloro-pyrrolo[2,3-d]pyrimidine (102a)

To a solution of (R)-[(3aR,4R,6R,6aR)-4-(4-chloropyrrolo[2,3-d]pyrimidin-7-yl)-2,2-dimethyl-3a,4,6,6a-tetrahydrofuro[3,4-d][1,3]dioxol-6-yl]-(4-chloro-3-fluoro-phenyl)methanol (800.0 mg, 1.76 mmol) in DMF (12.0 mL) was added iodomethane (299.9 mg, 2.11 mmol), then added sodium hydride (105.66 mg, 2.64 mmol) at 0° C., then the mixture warmed to 25° C. naturally and stirred at 25° C. for 2 h. LCMS showed the reaction was completed. The reaction mixture was added NH₄Cl aqueous (20.00 mL), EA (150.00 mL). The reaction mixture was washed with H₂O (30.00 mL×4), dried over Na₂SO₄, filtered and concentrated in vacuum to give crude product which was purified by silica gel column chromatography (PE:EA=12:1) to give 7-[(3aR,4R,6R,6aR)-6-[(4-chloro-3-fluoro-phenyl)-methoxy-methyl]-2,2-dimethyl-3a,4,6,6a-tetrahydrofuro[3,4-d][1,3]dioxol-4-yl]-4-chloro-pyrrolo[2,3-d]pyrimidine (102a) (690.0 mg, 1.47 mmol, 83.7% yield). LCMS [M+H]: 468.3.

### b) Synthesis of compound (2S,3S,4R,5R)-2-[(R)-(4-chloro-3-fluoro-phenyl)-methoxy-methyl]-5-[(4Z)-4-methoxyimino-1H-pyrrolo[2,3-d]pyrimidin-7-yl]tetrahydrofuran-3,4-diol hydrochloride (102)

To a solution of 7-[(3aR,4R,6R,6aR)-6-[(R)-(4-chloro-3-fluoro-phenyl)-methoxy-methyl]-2,2-dimethyl-3a,4,6,6a-tetrahydrofuro[3,4-d][1,3]dioxol-4-yl]-4-chloro-pyrrolo[2,3-d]pyrimidine (102a) (250.0 mg, 0.53 mmol) in 1-butanol (8.0 mL) was added O-Methylhydroxylamine hydrochloride (222.9 mg, 2.67 mmol), K₂CO₃ (589.35 mg, 4.27 mmol). The reaction mixture was stirred at 100° C. for 2 h. LCMS showed the reaction was completed. The reaction mixture was sent to pre-HPLC to give (2S,3S,4R,5R)-2-[(R)-(4-chloro-3-fluoro-phenyl)-methoxy-methyl]-5-[(4Z)-4-methoxyimino-1H-pyrrolo[2,3-d]pyrimidin-7-yl]tetrahydrofuran-3,4-diol hydrochloride (Ex. 102) (120.0 mg, 0.25 mmol, 47.1% yield) as a white solid. LCMS [M+H]: 439.1. ¹H NMR (400 MHz, DMSO-d₆+D₂O): δ 8.21 (s, 1H), 7.53-7.57 (m, 2H), 7.31-7.34 (m, 1H), 7.20-7.22 (m, 1H), 6.73-6.74 (m, 1H), 6.07-6.09 (m, 1H), 4.46-4.51 (m, 2H), 4.14-4.15 (m, 1H), 3.99-4.00 (m, 1H), 3.85 (s, 3H), 3.24 (s, 3H).

### Example 103. 7-((2R,3R,4S,5R)-5-((R)-(3,4-dichlorophenyl)(hydroxy)methyl)-3,4-dihydroxytetrahydrofuran-2-yl)-1,4a,7,7a-tetrahydro-4H-pyrrolo[2,3-d]pyrimidin-4-one 0-(2,2,2-trifluoroethyl) oxime (103)

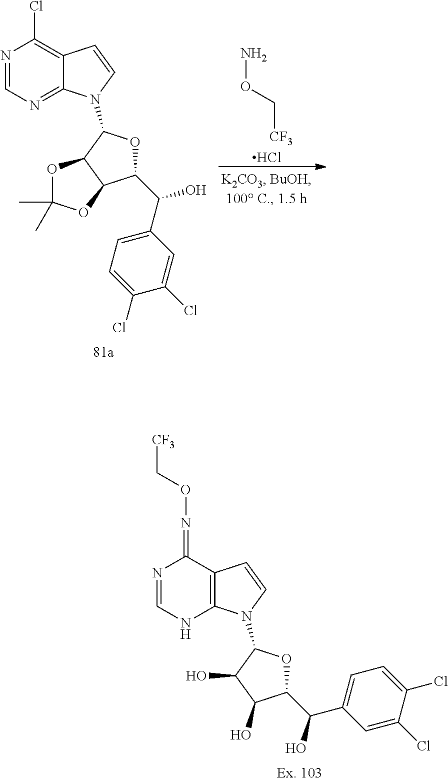 [SMILES: CC1(C)O[C@H]2[C@@H](O1)[C@H](n1ccc3c(Cl)ncnc31)O[C@@H]2[C@H](O)c1ccc(Cl)c(Cl)c1 ; NOCC(F)(F)F ; Cl ; O[C@@H]1[C@H](O)[C@@H]([C@H](O)c2ccc(Cl)c(Cl)c2)O[C@H]1n1ccc2c(=NOCC(F)(F)F)nc[nH]c21]

To a solution of (R)-[(3aR,4R,6R,6aR)-4-(4-chloropyrrolo[2,3-d]pyrimidin-7-yl)-2,2-dimethyl-3a, 4,6,6a-tetrahydrofuro[3,4-d][1,3]dioxol-6-yl]-(3,4-dichlorophenyl)methanol (81a) (199.6 mg, 0.42 mmol) in 1-Butanol (16.0 mL), Potassium carbonate (351.6 mg, 2.54 mmol) was added. The mixture was stirred at 100° C. for 1.5 h. LCMS showed the reaction was completed. The mixture was filtered and concentrated. The residue was purified by prep-HPLC, eluted with CH₃CN in H₂O (0.1% TFA) from 5.0% to 95.0% to give the TFA salt of the desired product (132.1 mg). The TFA salt was dissolved in CH₃CN (3.0 mL) and was added 1 M HCl aq. (4.0 mL). The solution was concentrated and lyophilized to give (2R,3S,4R,5R)-2-[(R)-(3,4-dichlorophenyl)-hydroxy-methyl]-5-[(4Z)-4-(2,2,2-trifluoroethoxyimino)-1H-pyrrolo[2,3-d]pyrimidin-7-yl]tetrahydrofuran-3,4-diol hydrochloride (Ex. 103) (107.4 mg, 0.20 mmol, 46.4% yield) as an off-white solid. LCMS [M+H]: 509.1. ¹H NMR (400 M Hz, DMSO-d₆): δ 7.70 (s, 1H), 7.60 (d, J=1.6 Hz, 1H), 7.57 (d, J=8.4 Hz, 1H), 7.36-7.39 (m, 1H), 7.32 (d, J=3.2 Hz, 1H), 6.41 (s, 1H), 5.93 (d, J=7.6 Hz, 1H), 4.77 (d, J=5.2 Hz, 1H), 4.55 (q, J=9.2 Hz, 2H), 4.43-4.46 (m, 1H), 4.06 (d, J=4.8 Hz, 1H), 3.94 (d, J=4.8 Hz, 1H). ¹H NMR (400 M Hz, DMSO-d6+D₂O): δ 7.67 (s, 1H), 7.56-7.59 (m, 2H), 7.36-7.39 (m, 1H), 7.22 (d, J=3.2 Hz, 1H), 6.39 (d, J=3.2 Hz, 1H), 5.90 (d, J=7.2 Hz, 1H), 4.76 (d, J=5.2 Hz, 1H), 4.43-4.54 (m, 3H), 4.07 (d, J=4.8 Hz, 1H), 3.97 (d, J=5.2 Hz, 1H). ¹⁹F NMR (376 M Hz, DMSO-d₆): δ −71.85 (s, 3F).

### Example 104. 7-((2R,3R,4S,5R)-5-((R)-(3,4-dichlorophenyl)(hydroxy)methyl)-3,4-dihydroxytetrahydrofuran-2-yl)-5-ethynyl-1,5-dihydro-4H-714-pyrrolo[2,3-d]pyrimidin-4-one O-methyl oxime (104)

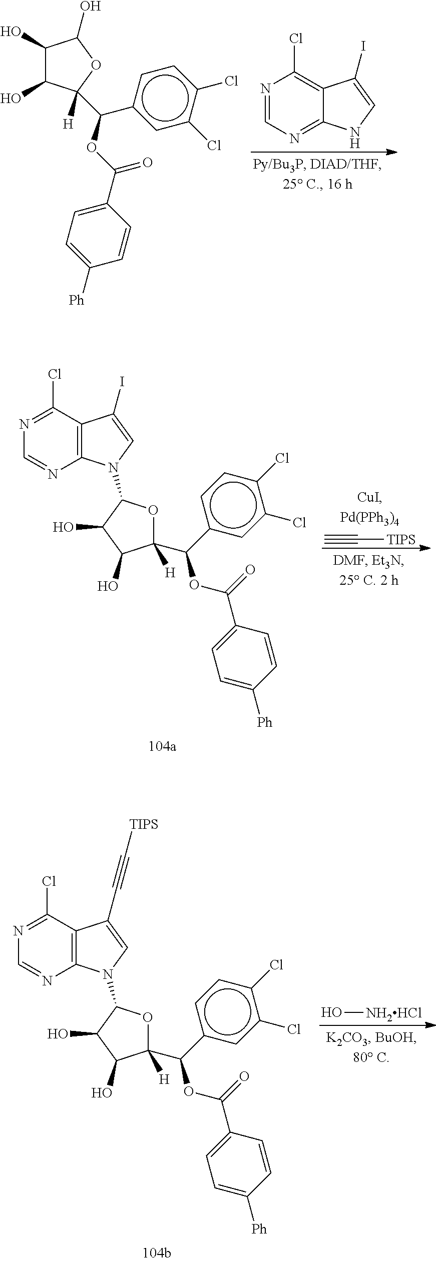

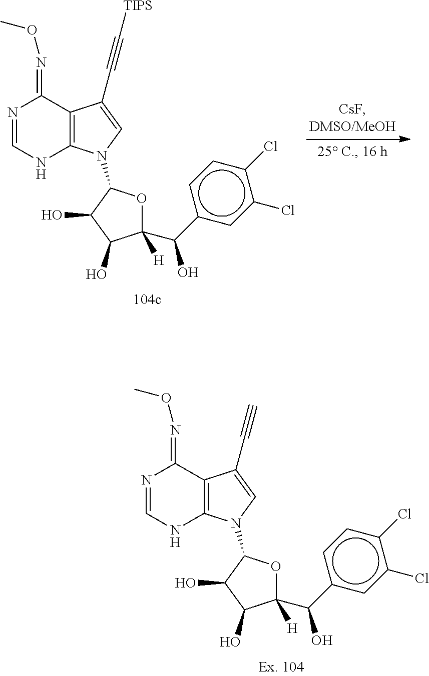

### a) Synthesis of [(R)-[(2S,3S,4R,5R)-5-(4-chloro-5-iodo-pyrrolo[2,3-d]pyrimidin-7-yl)-3,4-dihydroxy-tetrahydrofuran-2-yl]-(3,4-dichloro-phenyl)methyl] 4-phenylbenzoate (104a)

To a solution of 4-chloro-5-iodo-7H-pyrrolo[2,3-d]pyrimidine (1.76 g, 6.31 mmol) in THE (20.0 mL) was added Pyridine (0.5 mL, 6.31 mmol), Diisopropyl azodicarboxylate (2.68 mL, 13.25 mmol), followed by Tributylphosphine (3.2 mL, 12.62 mmol). [(R)-(3,4-dichlorophenyl)-[(2S,3S,4R,5R)-3,4,5-trihydroxytetrahydrofuran-2-yl]methyl]-4-phenyl-benzoate (3.0 g, 6.31 mmol) was added all at once at 25° C. The reaction mixture was stirred at 25° C. for 16 h under N₂. The solvent was removed in vacuum and the residue was purified by prep-HPLC to give [(R)-[(2S,3S,4R,5R)-5-(4-chloro-5-iodo-pyrrolo[2,3-d]pyrimidin-7-yl)-3,4-dihydroxy-tetrahydrofuran-2-yl]-(3,4-dichloro-phenyl)methyl] 4-phenylbenzoate (104a) (2.80 g, 2.81 mmol, 44.6% yield) as a pale yellow solid. LCMS [M+H]: 736.2

### b) Synthesis of [(R)-[(2S,3S,4R,5R)-5-[4-chloro-5-(2-triisopropylsilylethynyl)pyrrolo[2,3-d]pyrimidin-7-yl]-3,4-dihydroxy-tetrahydrofuran-2-yl]-(3,4-dichlorophenyl)methyl]4-phenylbenzoate (104b)

To a solution of [(R)-[(2S,3 S,4R,5R)-5-(4-chloro-5-iodo-pyrrolo[2,3-d]pyrimidin-7-yl)-3,4-dihydroxy-tetrahydrofuran-2-yl]-(3,4-dichlorophenyl)methyl]-4-phenylbenzoate (104a) (1.5 g, 2.04 mmol) in DMF (20.0 mL) was added TEA (0.62 g, 6.11 mmol), CuI (0.04 g, 0.20 mmol) and ethynyl(triisopropyl)silane (0.56 g, 3.05 mmol), Pd(PPh₃)₄ (0.12 g, 0.10 mmol). The reaction mixture was stirred at 25° C. for 2 h under N₂. LCMS showed the reaction was completed and no SM was left. The reaction mixture was diluted with EA (150.0 mL) and washed with water (100.0 mL×3) and brine (100 mL×3). The organic layer was dried over anhydrous Na₂SO₄, filtered and concentrated in vacuum to give crude product, which was purified by silica gel column chromatography (EA:PE=50:1 to 20:1) to give [(R)-[(2S,3S,4R,5R)-5-[4-chloro-5-(2-triisopropylsilylethynyl)pyrrolo[2,3-d]pyrimidin-7-yl]-3,4-dihydroxy-tetrahydrofuran-2-yl]-(3,4-dichloro phenyl)methyl]4-phenylbenzoate (104b) (1.0 g, 1.26 mmol, 62.1% yield) as a yellow oil. LCMS [M+H]: 790.0.

### c) Synthesis of (2R,3S,4R,5R)-2-[(R)-(3,4-dichlorophenyl)-hydroxy-methyl]-5-[(4Z)-4-methoxyimino-5-(2-triisopropylsilylethynyl)-1H-pyrrolo[2,3-d]pyrimidin-7-yl]tetrahydrofuran-3,4-diol (104c)

To a solution of [(R)-[(2S,3S,4R,5R)-5-[4-chloro-5-(2-triisopropylsilylethynyl)pyrrolo[2,3-d]pyrimidin-7-yl]-3,4-dihydroxy-tetrahydrofuran-2-yl]-(3,4-dichlorophenyl)methyl]4-phenylbenzoate (104b) (1.0 g, 1.26 mmol) in 1-Butanol (20.0 mL) was added O-Methylhydroxylamine hydrochloride (0.56 g, 6.32 mmol) and K₂CO₃ (1.4 g, 10.11 mmol). The reaction mixture was stirred at 80° C. for 6 h. The reaction was monitored by TLC (PE:EA=1:1, R_{f}=0.4), it showed the reaction was completed. The reaction mixture was filtered and concentrated in vacuum to give (2R,3S,4R,5R)-2-[(R)-(3,4-dichlorophenyl)-hydroxy-methyl]-5-[(4Z)-4-methoxyimino-5-(2-triisopropylsilylethynyl)-1H-pyrrolo[2,3-d]pyrimidin-7-yl]tetrahydrofuran-3,4-diol (104c) (1.25 g, 0.80 mmol, 63.6% yield) as a pale yellow solid.

### d) Synthesis of (2R,3S,4R,5R)-2-[(R)-(3,4-dichlorophenyl)-hydroxy-methyl]-5-[(4Z)-5-ethynyl-4-methoxyimino-1H-pyrrolo[2,3-d]pyrimidin-7-yl]tetrahydrofuran-3,4-diol (Ex. 104)

To a solution of (2R,3S,4R,5R)-2-[(R)-(3,4-dichlorophenyl)-hydroxy-methyl]-5-[(4Z)-4-methoxyimino-5-(2-triisopropylsilylethynyl)-1H-pyrrolo[2,3-d]pyrimidin-7-yl]tetrahydrofuran-3,4-diol (104c) (1.25 g, 0.80 mmol) in DMSO (20.0 mL) and Methanol (0.10 mL) was added CsF (122.18 mg, 0.80 mmol). The reaction mixture was stirred at 25° C. for 16 h. LCMS showed the reaction was completed. The reaction mixture was filtered and concentrated in vacuum to give crude product which was purified by prep-HPLC, eluted with CH₃CN in H₂O (0.1% NH₃.H₂O) from 10.0% to 95.0%) to obtain (2R,3S,4R,5R)-2-[(R)-(3,4-dichlorophenyl)-hydroxy-methyl]-5-[(4Z)-5-ethynyl-4-methoxyimino-1H-pyrrolo[2,3-d]pyrimidin-7-yl]tetrahydrofuran-3,4-diol (Ex. 104) (130.0 mg, 0.26 mmol, 32.2% yield) as a pale yellow solid. LCMS [M+H]: 465.3. ¹H NMR (400 M Hz, DMSO-d₆): δ 11.03 (s, 1H), 7.61-7.62 (m, 1H), 7.57-7.59 (m, 2H), 7.48-7.52 (m, 1H), 7.37-7.39 (m, 1H), 6.13-6.14 (m, 1H), 5.84-5.86 (d, J=7.6 Hz, 1H), 5.28-5.30 (d, J=6.4 Hz, 1H), 5.08-5.09 (d, J=3.6 Hz, 1H), 4.77-4.78 (m, 1H), 3.36-4.40 (m, 1H), 4.02 (m, 1H), 3.93-3.94 (m, 1H), 3.91 (s, 1H), 3.73 (s, 3H). ¹H NMR (400 M Hz, DMSO-d₆+D₂O): δ 7.61 (m, 1H), 7.57-7.60 (d, J=8.4 Hz, 1H), 7.50 (m, 2H), 7.37-7.39 (m, 1H), 5.84-5.86 (d, J=7.2 Hz, 1H), 4.76-4.78 (d, J=9.2 Hz, 1H), 4.37-4.40 (m, 1H), 4.02-4.03 (m, 1H), 3.94-3.96 (m, 1H), 3.89 (s, 1H), 3.74 (s, 3H).

### Example 105. 7-((2R,3R,4S,5S)-5-((1R)-1-(3,4-dichlorocyclohexa-2,4-dien-1-yl)-1-hydroxyethyl)-3,4-dihydroxytetrahydrofuran-2-yl)-1,7-dihydro-4H-pyrrolo[2,3-d]pyrimidin-4-one oxime (105)

Example 105 (TFA salt) was prepared similarly to that of Ex. 61. ¹H NMR (600 MHz, Methanol-d₄) δ 8.27 (s, 1H), 7.73 (dd, J=2.9, 10.1 Hz, 2H), 7.51 (d, J=8.4 Hz, 1H), 7.45 (dd, J=2.1, 8.4 Hz, 1H), 6.77 (d, J=3.7 Hz, 1H), 6.20 (d, J=7.2 Hz, 1H), 4.57 (dd, J=5.3, 7.2 Hz, 1H), 4.23 (d, J=1.8 Hz, 1H), 3.97 (dd, J=1.8, 5.3 Hz, 1H), 1.56 (s, 3H).

### Example 106. 7-((2R,3R,4S,5R)-5-((R)-(3,4-dichlorophenyl)(hydroxy)methyl)-3,4-dihydroxytetrahydrofuran-2-yl)-1,4a,7,7a-tetrahydro-4H-pyrrolo[2,3-d]pyrimidin-4-one 0-(2,2-difluoroethyl) oxime (106)

Example 106 was prepared similarly to that of Ex. 66. LCMS [M+H]: 491.2. ¹H NMR (400 MHz, DMSO-d₆+D₂O) δ 7.85 (s, 1H), 7.56-7.61 (m, 2H), 7.36-7.39 (m, 2H), 6.51 (d, J=3.2 Hz, 1H), 6.16-6.46 (m, 1H), 5.96 (d, J=7.6 Hz, 1H), 4.77 (d, J=5.2 Hz, 1H), 4.45-4.48 (m, 1H), 4.18-4.26 (m, 2H), 4.07 (d, J=4.8 Hz, 1H) 3.97 (d, J=4.8 Hz, 1H). ¹⁹FNMR (376 MHz, DMSO-d6) δ −125.497 (s, 2F).

### Example 107. 7-((2R,3R,4S,5S)-5-((R)-1-(3,4-dichlorophenyl)-1-hydroxyethyl)-3,4-dihydroxytetrahydrofuran-2-yl)-5-fluoro-1,7-dihydro-4H-pyrrolo[2,3-d]pyrimidin-4-one O-methyl oxime (107)

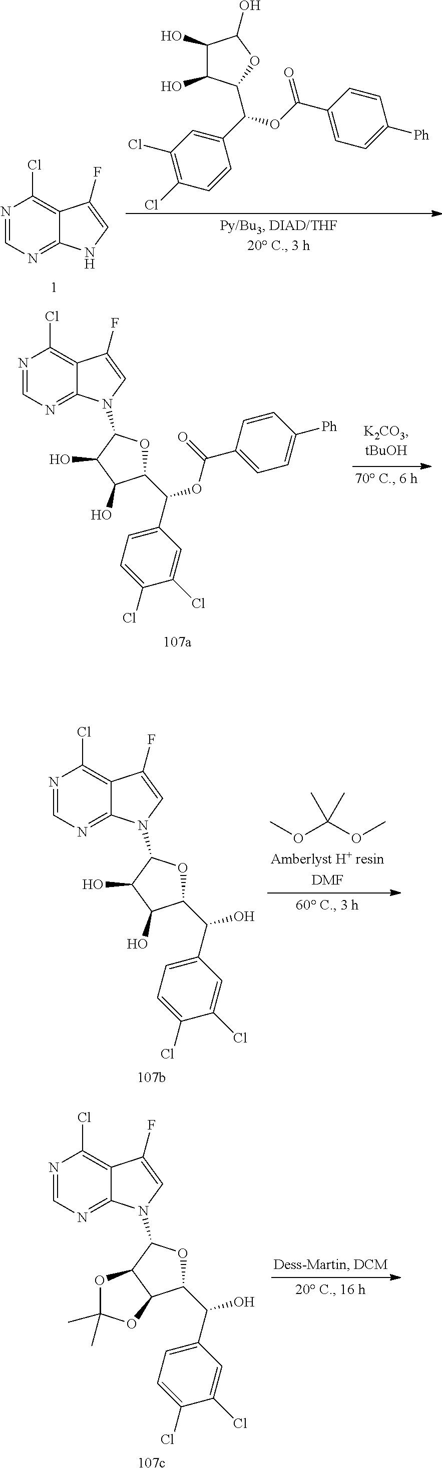

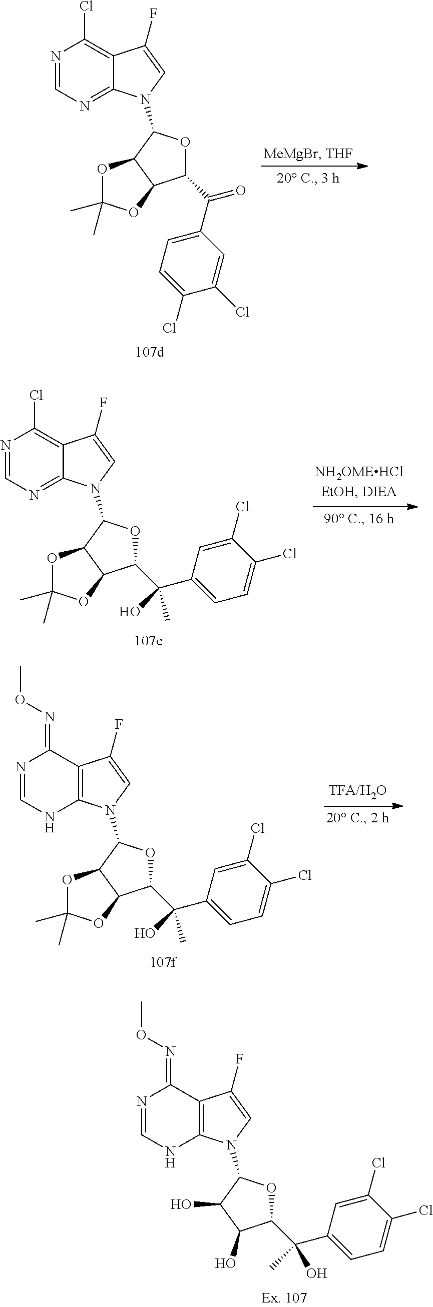 [SMILES: CC1(C)O[C@@H]2[C@H](O1)[C@@H](C(=O)c1ccc(Cl)c(Cl)c1)O[C@H]2n1cc(F)c2c(Cl)ncnc21 ; CC1(C)O[C@@H]2[C@H](O1)[C@@H]([C@](C)(O)c1ccc(Cl)c(Cl)c1)O[C@H]2n1cc(F)c2c(Cl)ncnc21 ; CO/N=c1\nc[nH]c2c1c(F)cn2[C@@H]1O[C@H]([C@](C)(O)c2ccc(Cl)c(Cl)c2)[C@H]2OC(C)(C)O[C@H]21 ; CO/N=c1\nc[nH]c2c1c(F)cn2[C@@H]1O[C@H]([C@](C)(O)c2ccc(Cl)c(Cl)c2)[C@@H](O)[C@H]1O]

### a) Synthesis of [(R)-[(2S,3S,4R,5R)-5-(4-chloro-5-fluoro-pyrrolo[2,3-d]pyrimidin-7-yl)-3,4-dihydroxy-tetrahydrofuran-2-yl]-(3,4-dichlorophenyl)methyl] 4-phenylbenzoate (107a)

To a solution of 4-chloro-5-fluoro-7H-pyrrolo[2,3-d]pyrimidine (1.08 g, 6.31 mmol), pyridine (0.5 mL, 6.31 mmol), PBu₃ (3.1 mL, 12.62 mmol) in THF (35.0 mL) was quickly added DIAD (2.4 mL, 12.62 mmol) and [(R)-(3,4-dichlorophenyl)-[(2S,3S,4R)-3,4,5-trihydroxytetrahydrofuran-2-yl]-methyl] 4-phenylbenzoate (3.00 g, 6.31 mmol) in THF at 0° C. under N₂. The mixture was stirred at 20° C. for 3 h. The solvent was removed in vacuum. The residue was purified by reversed-phase combi-flash, eluted with MeCN in H₂O from 20.0% to 80.0% to give [(R)-[(2S,3S,4R,5R)-5-(4-chloro-5-fluoro-pyrrolo[2,3-d]pyrimidin-7-yl)-3,4-dihydroxy-tetrahydrofuran-2-yl]-(3,4-dichlorophenyl)methyl] 4-phenylbenzoate (107a) (2.50 g, 3.97 mmol, 62.9% yield) as a light yellow solid. LCMS [M+H]: 628.1.

### b) Synthesis of (2R,3R,4S,5R)-2-(4-chloro-5-fluoro-pyrrolo[2,3-d]pyrimidin-7-yl)-5-[(R)-(3,4dichlorophenyl)-hydroxy-methyl]tetrahydrofuran-3,4-diol (107b)

To a solution of [(R)-[(2S,3S,4R,5R)-5-(4-chloro-5-fluoro-pyrrolo[2,3-d]pyrimidin-7-yl)-3,4-dihydroxy-tetrahydrofuran-2-yl]-(3,4-dichlorophenyl)methyl] 4-phenylbenzoate (107a) (1.50 g, 2.39 mmol) in tert-butanol (20.0 mL) was added K₂CO₃ (1.97 g, 14.31 mmol) under N₂. The mixture was stirred at 70° C. for 6 h. The solvent was removed in vacuum to give crude product which was purified by reversed-phase combi-flash, eluted with CH₃CN in H₂O (neutral condition) from 10.0% to 85.0% to afford (2R,3R,4S,5R)-2-(4-chloro-5-fluoro-pyrrolo[2,3-d]pyrimidin-7-yl)-5-[(R)-(3,4-dichlorophenyl)-hydroxy-methyl]tetrahydrofuran-3,4-diol (107b) (0.75 g, 1.67 mmol, 70.1% yield) as a yellow solid. LCMS [M+H]: 448.1.

### c) Synthesis of (R)-[(3aR,4R,6R,6aR)-4-(4-chloro-5-fluoro-pyrrolo[2,3-d]pyrimidin-7-yl)-2,2-dimethyl-3a,4,6,6a-tetrahydrofuro[3,4-d][1,3]dioxol-6-yl]-(3,4-dichlorophenyl)methanol (107c))

To a solution of (2R,3R,4S,5R)-2-(4-chloro-5-fluoro-pyrrolo[2,3-d]pyrimidin-7-yl)-5-[(R)-(3,4-dichlorophenyl)-hydroxy-methyl]tetrahydrofuran-3,4-diol (107b) (0.53 g, 1.18 mmol) in DMF (5.0 mL) was added amberlyst H+ resin (0.53 g) and 2,2-Dimethoxypropane (1.84 g, 17.72 mmol) under N₂. The mixture was stirred at 60° C. for 3 h. The solvent was removed in vacuum to give crude product which was purified by silica chromatography (PE:EA=10:1 to 2:1) to give (R)-[(3aR,4R,6R,6aR)-4-(4-chloro-5-fluoro-pyrrolo[2,3-d]pyrimidin-7-yl)-2,2-dimethyl-3a,4,6,6a-tetrahydrofuro[3,4-d][1,3]dioxol-6-yl]-(3,4-dichlorophenyl)methanol (107c) (0.23 g, 0.47 mmol, 39.8% yield) as a yellow solid. LCMS [M+H]: 488.1.

### d) Synthesis of [(3aR,4R,6S,6aS)-4-(4-chloro-5-fluoro-pyrrolo[2,3-d]pyrimidin-7-yl)-2,2-dimethyl-3a,4,6,6a-tetrahydrofuro[3,4-d][1,3]dioxol-6-yl]-(3,4-dichlorophenyl)methanone (107d)

To a solution of (R)-[(3aR,4R,6R,6aR)-4-(4-chloro-5-fluoro-pyrrolo[2,3-d]pyrimidin-7-yl)-2,2-dimethyl-3a,4,6,6a-tetrahydrofuro[3,4-d][1,3]dioxol-6-yl]-(3,4-dichlorophenyl)methanol (107c) (230.0 mg, 0.47 mmol) in DCM (5.0 mL) was added Dess-Martin periodinane (499.0 mg, 1.18 mmol) under N₂. The mixture was stirred at 20° C. for 16 h. The solvent was removed in vacuum to give the crude product, which was purified by silica chromatography (PE:EA=10:1 to 2:1) to give [(3aR,4R,6S,6aS)-4-(4-chloro-5-fluoro-pyrrolo[2,3-d]pyrimidin-7-yl)-2,2-dimethyl-3a,4,6,6a-tetrahydrofuro[3,4-d][1,3]dioxol-6-yl]-(3,4-dichlorophenyl)methanone (107d) (200.0 mg, 0.41 mmol, 87.3% yield) as a yellow solid. LCMS [M+H]: 486.1.

### e) Synthesis of (1R)-1-[(3aR,4R,6S,6aR)-4-(4-chloro-5-fluoro-pyrrolo[2,3-d]pyrimidin-7-yl)-2,2-dimethyl-3a,4,6,6a-tetrahydrofuro[3,4-d][1,3]dioxol-6-yl]-1-(3,4-dichlorophenyl)ethanol (107e)

To a solution of [(3aR,4R,6S,6aS)-4-(4-chloro-5-fluoro-pyrrolo[2,3-d]pyrimidin-7-yl)-2,2-dimethyl-3a,4,6,6a-tetrahydrofuro[3,4-d][1,3]dioxol-6-yl]-(3,4-dichlorophenyl)methanone (107d) (200.0 mg, 0.41 mmol) in THE (2.0 mL) was added MeMgBr (0.4 mL, 1.23 mmol) under N₂. The mixture was stirred at 20° C. for 3 h. The reaction was quenched with sat. aq. NH₄Cl and extracted with EA. The organic layers were dried with Na₂SO₄ and filtered. The filtrates were concentrated in vacuum to give the crude product, which was purified by silica chromatography (PE:EA=10:1 to 2:1) to afford (1R)-1-[(3aR,4R,6S,6aR)-4-(4-chloro-5-fluoro-pyrrolo[2,3-d]pyrimidin-7-yl)-2,2-dimethyl-3a,4,6,6a-tetrahydrofuro[3,4-d][1,3]dioxol-6-yl]-1-(3,4-dichlorophenyl)ethanol (107e) (70.0 mg, 0.14 mmol, 33.9% yield) as a yellow solid. LCMS [M+H]: 502.1.

### f) Synthesis of (1R)-1-[(3aR,4R,6S,6aR)-4-[(4Z)-5-fluoro-4-methoxyimino-1H-pyrrolo[2,3-d]pyrimidin-7-yl]-2,2-dimethyl-3a,4,6,6a-tetrahydrofuro[3,4-d][1,3]dioxol-6-yl]-1-(3,4-dichlorophenyl)ethanol (107f)

To a solution of (1R)-1-[(3aR,4R,6S,6aR)-4-(4-chloro-5-fluoro-pyrrolo[2,3-d]pyrimidin-7-yl)-2,2-dimethyl-3a,4,6,6a-tetrahydrofuro[3,4-d][1,3]dioxol-6-yl]-1-(3,4-dichlorophenyl)ethanol (107e) (70.0 mg, 0.14 mmol) in Ethanol (3.0 mL) was added O-Methylhydroxylamine hydrochloride (69.7 mg, 0.84 mmol) and DIEA (143.9 mg, 1.11 mmol). The reaction mixture was stirred at 90° C. for 16 h. The solvent was removed in vacuum to give crude product which was purified by reversed-phase combi-flash, eluted with MeCN in H₂O from 10.0% to 70.0% to give (1R)-1-[(3aR,4R,6S,6aR)-4-[(4Z)-5-fluoro-4-methoxyimino-1H-pyrrolo[2,3-d]pyrimidin-7-yl]-2,2-dimethyl-3a,4,6,6a-tetrahydrofuro[3,4-d][1,3]dioxol-6-yl]-1-(3,4-dichlorophenyl)ethanol (107f) (40.0 mg, 0.08 mmol, 55.9% yield) as a gray solid. LCMS [M+H]: 513.1.

### g) Synthesis of (2S,3S,4R,5R)-2-[(1R)-1-(3,4-dichlorophenyl)-1-hydroxy-ethyl]-5-[(4Z)-5-fluoro-4-methoxyimino-1H-pyrrolo[2,3-d]pyrimidin-7-yl]tetrahydrofuran-3,4-diol hydrochloride (Ex. 107)

A mixture of (1R)-1-[(3aR,4R,6S,6aR)-4-[(4Z)-5-fluoro-4-methoxyimino-1H-pyrrolo[2,3-d] pyrimidin-7-yl]-2,2-dimethyl-3a,4,6,6a-tetrahydrofuro[3,4-d][1,3]dioxol-6-yl]-1-(3,4-dichlorophenyl)ethanol (107f) (40.0 mg, 0.08 mmol) in Water (1.0 mL) and TFA (0.5 mL, 6.73 mmol) was stirred at 20° C. for 2 h. The solvent was removed in vacuum to give the crude product, which was purified by prep-HPLC, eluted with MeCN in H₂O (0.1% TFA) from 5.0% to 80.0%. The fractions were combined and 5 drops of 1M HCl were added, then lyophilized to afford (2S,3 S,4R,5R)-2-[(1R)-1-(3,4-dichlorophenyl)-1-hydroxy-ethyl]-5-[(4Z)-5-fluoro-4-methoxyimino-1H-pyrrolo[2,3-d]pyrimidin-7-yl]tetrahydrofuran-3,4-diol hydrochloride (Ex. 107) (9.5 mg, 0.018 mmol, 23.8% yield) as a white solid. LCMS [M+H]: 473.1. ¹H NMR (400 MHz, DMSO-d₆+D₂O) δ 7.73-7.70 (m, 2H), 7.61 (d, J=8.4 Hz, 1H), 7.49 (d, J=8.4 Hz, 1H), 7.23 (s, 1H), 5.93 (d, J=7.7 Hz, 1H), 4.28-4.25 (m, 1H), 4.04 (brs, 1H), 3.77 (s, 3H), 3.69 (d, J=5.1 Hz, 1H), 1.44 (s, 3H). ¹⁹FNMR (377 MHz, DMSO-d₆) δ −163.6 (s, 1F)

### Example 108. 9-((2R,3R,4S,5S)-5-((R)-1-(3,4-dichlorophenyl)-1-hydroxyethyl)-3,4-dihydroxytetrahydrofuran-2-yl)-3,9-dihydro-6H-purin-6-one oxime (108)

Example 108 was prepared similarly to that of Ex. 92. LCMS [M+H]: 442.2. ¹H NMR (400 M Hz, DMSO-d₆): δ 8.92 (s, 1H), 8.33 (s, 1H), 8.07 (s, 1H), 7.79 (d, J=2.0 Hz, 1H), 7.61-7.63 (m, 1H), 7.52-7.55 (m, 1H), 5.87 (d, J=7.6 Hz, 1H), 4.50-4.54 (m, 1H), 4.19 (s, 1H), 3.70 (d, J=4.8 Hz, 1H), 1.44 (s, 3H). ¹H NMR (400 M Hz, DMSO-d₆+D₂O): δ 8.37 (s, 1H), 8.12 (s, 1H), 7.77 (d, J=2.0 Hz, 1H), 7.61-7.63 (m, 1H), 7.51-7.54 (m, 1H), 5.89 (d, J=7.6 Hz, 1H), 4.49-4.53 (m, 1H), 4.18 (s, 1H), 3.72 (d, J=4.8 Hz, 1H), 1.45 (s, 3H).

### X-Ray Crystal Structure of Example 69

Compound of Example 69 was recrystallized from ethanol. Crystals suitable for X-ray diffraction studies were obtained as clear colorless prisms. A clear colourless block-like specimen of C20H24Cl₂N4O6, approximate dimensions 0.144 mm×0.214 mm×0.451 mm, was used for the X-ray crystallographic analysis. Table 1 shows the corresponding data collection details.

TABLE 1
Data collection details for Example 69.

| | | | | | | | | | wavelength/ | | | |
|---|---|---|---|---|---|---|---|---|---|---|---|---|
| axis | dx/mm | 20/° | ω/° | φ/° | χ/° | width/° | frames | time/s | Å | volt/kV | current/mA | temp/K |
| Omega | 49.466 | 118.90 | −75.10 | 24.00 | 54.74 | 1.30 | 160 | 20.00 | 1.54184 | 45 | 0.7 | n/a |
| Omega | 49.466 | −58.14 | 107.86 | 270.00 | 54.74 | 1.30 | 160 | 20.00 | 1.54184 | 45 | 0.7 | n/a |
| Omega | 49.466 | 118.90 | −75.10 | 168.00 | 54.74 | 1.30 | 160 | 20.00 | 1.54184 | 45 | 0.7 | n/a |
| Omega | 49.466 | −58.14 | 107.86 | 180.00 | 54.74 | 1.30 | 160 | 20.00 | 1.54184 | 45 | 0.7 | n/a |
| Omega | 49.466 | 118.00 | −76.00 | 81.35 | 54.79 | 1.30 | 160 | 20.00 | 1.54184 | 45 | 0.7 | n/a |
| Omega | 49.466 | 118.90 | −75.10 | −24.00 | 54.74 | 1.30 | 160 | 20.00 | 1.54184 | 45 | 0.7 | n/a |
| Omega | 49.466 | −73.90 | 93.40 | 0.00 | 54.74 | 1.30 | 159 | 20.00 | 1.54184 | 45 | 0.7 | n/a |
| Omega | 49.466 | 118.90 | −75.10 | 48.00 | 54.74 | 1.30 | 160 | 20.00 | 1.54184 | 45 | 0.7 | n/a |
| Omega | 49.466 | 118.90 | −75.10 | 144.00 | 54.74 | 1.30 | 160 | 20.00 | 1.54184 | 45 | 0.7 | n/a |
| Omega | 49.466 | −28.84 | 137.15 | −120.00 | 54.74 | 1.30 | 160 | 20.00 | 1.54184 | 45 | 0.7 | n/a |
| Omega | 49.466 | −73.14 | 92.86 | 135.00 | 54.74 | 1.30 | 160 | 20.00 | 1.54184 | 45 | 0.7 | n/a |
| Omega | 49.466 | 118.90 | −75.10 | −96.00 | 54.74 | 1.30 | 160 | 20.00 | 1.54184 | 45 | 0.7 | n/a |
| Omega | 49.466 | 118.90 | −75.10 | 0.00 | 54.74 | 1.30 | 160 | 20.00 | 1.54184 | 45 | 0.7 | n/a |

A total of 2571 frames were collected. The total exposure time was 14.28 hours. The frames were integrated with the Bruker SAINT software package using a narrow-frame algorithm. The integration of the data using a monoclinic unit cell yielded a total of 14043 reflections to a maximum θ angle of 75.35° (0.80 Å resolution), of which 4554 were independent (average redundancy 3.084, completeness=99.3%, Rint=2.75%, Rsig=2.86%) and 4447 (97.65%) were greater than 2σ(F2). The final cell constants of a=25.6495(5) Å, b=6.93520(10) Å, c=15.3927(3) Å, β=125.5400(10)°, volume=2228.04(7) Å3, are based upon the refinement of the XYZ-centroids of 9909 reflections above 20 σ(I) with 7.057°<2θ<150.8°. Data were corrected for absorption effects using the Multi-Scan method (SADABS). The ratio of minimum to maximum apparent transmission was 0.734. The calculated minimum and maximum transmission coefficients (based on crystal size) are 0.5532 and 0.7539.

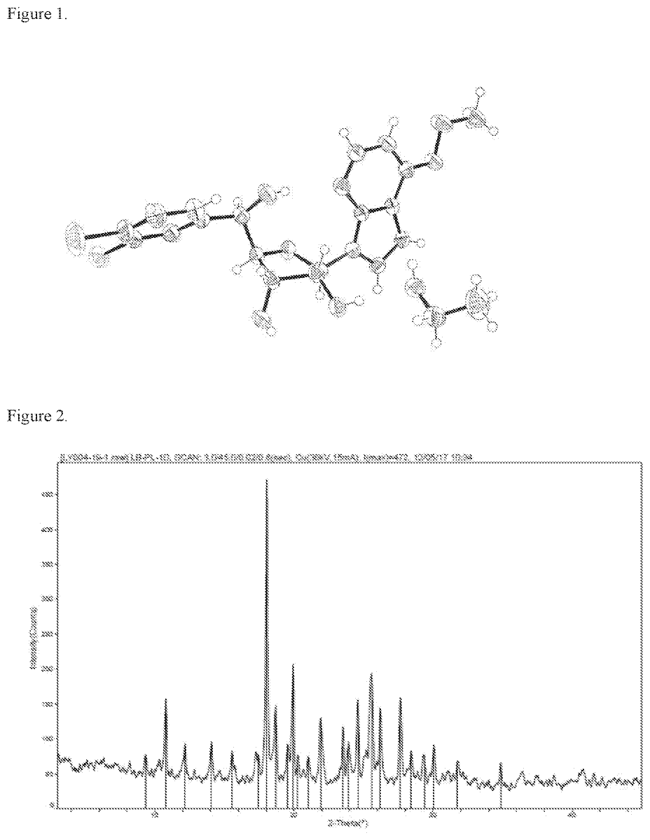

FIG. 1 is an ORTEP representation of Example 69. The structure was solved and refined using the Bruker SHELXTL Software Package, using the space group C 1 2 1, with Z=4 for the formula unit, C20H24Cl2N4O6. The final anisotropic full-matrix least-squares refinement on F2 with 307 variables converged at R1=2.97%, for the observed data and wR2=9.11% for all data. The goodness-of-fit was 1.016. The largest peak in the final difference electron density synthesis was 0.191 e−/Å3 and the largest hole was −0.210 e−/Å3 with an RMS deviation of 0.033 e−/Å3. On the basis of the final model, the calculated density was 1.453 g/cm3 and F(000), 1016 e−.

TABLE 2
Sample and crystal data for Example 69.

| Chemical formula | C20H24Cl2N4O6 | |
|---|---|---|
| Formula weight | 487.33 g/mol | |
| Temperature | 296(2) K | |
| Wavelength | 1.54178 Å | |
| Crystal size | 0.144 × 0.214 × 0.451 mm | |
| Crystal habit | clear colourless block | |
| Crystal system | monoclinic | |
| Space group | C 1 2 1 | |
| Unit cell dimensions | a = 25.6495(5) Å | α = 90° |
| | b = 6.93520(10) Å | β = 125.5400(10)° |
| | c = 15.3927(3) Å | γ = 90° |
| Volume | 2228.04(7) Å3 | |
| Z | 4 | |
| Density (calculated) | 1.453 g/cm3 | |
| Absorption coefficient | 3.018 mm−1 | |
| F(000) | 1016 | |

TABLE 3
Data collection and structure refinement for Example 69.

| | |
|---|---|
| Theta range for data collection | 3.53 to 75.35° |
| Index ranges | −32 <= h <= 32, −8 <= k <= 8, −19 <= 1 <= 18 |
| Reflections collected | 14043 |
| Independent reflections | 4554 [R(int) = 0.0275] |
| Coverage of independent reflections | 99.3% |
| Absorption correction | Multi-Scan |
| Max. and min. transmission | 0.7539 and 0.5532 |
| Structure solution technique | direct methods |
| Structure solution program | SHELXS-97 (Sheldrick 2008) |
| Refinement method | Full-matrix least-squares on F2 |
| Refinement program | SHELXL-2016/6 (Sheldrick, 2016) |
| Function minimized | Σ w(Fo2-Fc2)2 |
| Data/restraints/parameters | 4554/1/307 |
| Goodness-of-fit on F2 | 1.016 |
| Final R indices | 4447 data; I>2σ(I) R1 = 0.0297, wR2 = 0.0903 |
| | all data R1 = 0.0303, wR2 = 0.0911 |
| Weighting scheme | w = l/[σ2(Fo2) + (0.0672P)2 + 0.2961P] |
| where P = (Fo2 + 2Fc2)/3 | |
| Absolute structure parameter | −0.013(7) |
| Extinction coefficient | 0.0029(3) |
| Largest diff. peak and hole | 0.191 and −0.210 eÅ-3 |
| R.M.S. deviation from mean | 0.033 eÅ-3 |

TABLE 4
Atomic coordinates and equivalent isotropic atomic displacement parameters (Å2) for
Example 69. (U(eq) is defined as one third of the trace of the orthogonalized Uij tensor).

| | x/a | y/b | z/c | U(eq) |
|---|---|---|---|---|
| Cl1 | 0.35444(4) | 0.57767(14) | 0.82068(5) | 0.0688(2) |
| Cl2 | 0.42821(5) | 0.97062(19) | 0.86236(7) | 0.0947(4) |
| N1 | 0.28071(8) | 0.2013(3) | 0.20080(13) | 0.0347(3) |
| N2 | 0.14374(9) | 0.5353(3) | 0.96656(16) | 0.0418(4) |
| N3 | 0.22705(9) | 0.5149(3) | 0.15096(15) | 0.0397(4) |
| N4 | 0.11352(9) | 0.2768(3) | 0.84518(15) | 0.0425(4) |
| O1 | 0.28645(10) | 0.6992(3) | 0.35704(14) | 0.0540(4) |
| O2 | 0.28926(7) | 0.2759(2) | 0.35549(12) | 0.0393(3) |
| O3 | 0.43543(8) | 0.2818(3) | 0.54089(13) | 0.0501(4) |
| O4 | 0.43071(7) | 0.3157(3) | 0.36167(13) | 0.0467(4) |
| O5 | 0.06795(10) | 0.4174(3) | 0.77306(15) | 0.0608(5) |
| O6 | 0.42197(8) | 0.3845(3) | 0.17727(15) | 0.0467(4) |
| C1 | 0.32247(10) | 0.6008(4) | 0.62135(19) | 0.0431(5) |
| C2 | 0.35505(11) | 0.6879(4) | 0.72081(18) | 0.0469(5) |
| C3 | 0.38713(12) | 0.8593(5) | 0.7383(2) | 0.0547(6) |
| C4 | 0.38647(14) | 0.9446(4) | 0.6564(2) | 0.0571(6) |
| C5 | 0.35449(12) | 0.8564(4) | 0.5575(2) | 0.0484(5) |
| C6 | 0.32297(10) | 0.6826(3) | 0.53989(17) | 0.0395(4) |
| C7 | 0.29073(10) | 0.5765(3) | 0.43443(17) | 0.0410(4) |
| C8 | 0.32804(9) | 0.3923(3) | 0.44995(16) | 0.0348(4) |
| C9 | 0.39239(9) | 0.4216(3) | 0.46593(16) | 0.0375(4) |
| C10 | 0.37585(9) | 0.3794(3) | 0.35449(16) | 0.0350(4) |
| C11 | 0.32502(9) | 0.2233(3) | 0.31546(16) | 0.0342(4) |
| C12 | 0.27246(10) | 0.0310(3) | 0.14751(18) | 0.0389(4) |
| C13 | 0.22389(10) | 0.0518(3) | 0.04226(17) | 0.0377(4) |
| C14 | 0.19972(9) | 0.2416(3) | 0.02951(15) | 0.0328(4) |
| C15 | 0.14938(9) | 0.3491(3) | 0.94056(16) | 0.0357(4) |
| C16 | 0.18155(11) | 0.6061(3) | 0.06833(18) | 0.0410(4) |
| C17 | 0.23567(9) | 0.3300(3) | 0.12866(16) | 0.0327(4) |
| C18 | 0.03657(16) | 0.3483(5) | 0.6682(2) | 0.0659(8) |
| C19 | 0.48310(13) | 0.3875(5) | 0.1943(2) | 0.0589(7) |
| C20 | 0.47807(19) | 0.4426(9) | 0.0974(3) | 0.0883(12) |

TABLE 5
Hydrogen atomic coordinates and isotropic atomic
displacement parameters (Å2) for Example 69.

| | x/a | y/b | z/c | U(eq) |
|---|---|---|---|---|
| H2N | 0.1159(15) | 0.607(5) | −0.080(3) | 0.05 |
| H1O | 0.2679(19) | 0.649(6) | 0.306(3) | 0.065 |
| H3O | 0.4679(17) | 0.307(5) | 0.544(3) | 0.06 |
| H4O | 0.4277(16) | 0.339(5) | 0.309(3) | 0.056 |
| H6O | 0.4087(15) | 0.513(5) | 0.162(3) | 0.056 |
| H1A | 0.3002 | 0.4866 | 0.6093 | 0.052 |
| H4A | 0.4075 | 1.0612 | 0.6678 | 0.069 |
| H5A | 0.3541 | 0.9138 | 0.5025 | 0.058 |
| H7A | 0.2473 | 0.5409 | 0.4104 | 0.049 |
| H8A | 0.3354 | 0.3213 | 0.5112 | 0.042 |
| H9A | 0.4087 | 0.5530 | 0.4895 | 0.045 |
| H10A | 0.3575 | 0.4940 | 0.3090 | 0.042 |
| H11A | 0.3462 | 0.0995 | 0.3468 | 0.041 |
| H12A | 0.2964 | −0.0806 | 0.1788 | 0.047 |
| H13A | 0.2093 | −0.0405 | −0.0110 | 0.045 |
| H16A | 0.1736 | 0.7317 | 0.0788 | 0.049 |
| H18A | 0.0072 | 0.4443 | −0.3810 | 0.099 |
| H18B | 0.0135 | 0.2328 | −0.3400 | 0.099 |
| H18C | 0.0676 | 0.3205 | −0.3460 | 0.099 |
| H19A | 0.5025 | 0.2608 | 0.2172 | 0.071 |
| H19B | 0.5109 | 0.4782 | 0.2509 | 0.071 |
| H20A | 0.5186 | 0.4229 | 0.1086 | 0.133 |
| H20B | 0.4663 | 0.5761 | 0.0818 | 0.133 |
| H20C | 0.4460 | 0.3648 | 0.0385 | 0.133 |

### X-Ray Powder Diffraction (XRPD) of Example 92A

Characterization of the crystalline form prepared in Example 92A by an X-ray diffraction pattern using CuKa radiation as having diffraction peaks (2-theta values) (SCAN: 3.0/45.0/0.02/0.6 (sec), Cu(30 kV, 15 mA), J(max)=472) as described in Table 6 below.

TABLE 6
X-ray powder diffraction peaks of crystalline Example 92A
PEAK: 21-pts/Parabolic Filter, Threshold = 3.0, Cutoff = 0.1%, BG = 3/1.0, Peak-Top = Summit

| 2-Theta | d(A) | BG | Height | I % | Area | I % | FWHM |
|---|---|---|---|---|---|---|---|
| 9.321 | 9.4806 | 46 | 30 | 7 | 474 | 7.4 | 0.269 |
| 10.78 | 8.2 | 45 | 112 | 26.1 | 1591 | 24.9 | 0.241 |
| 12.178 | 7.2618 | 42 | 50 | 11.7 | 723 | 11.3 | 0.246 |
| 14.078 | 6.2857 | 40 | 54 | 12.6 | 869 | 13.6 | 0.274 |
| 15.56 | 5.6902 | 39 | 43 | 10 | 637 | 10 | 0.252 |
| 17.44 | 5.0807 | 49 | 28 | 6.5 | 492 | 7.7 | 0.299 |
| 18.058 | 4.9082 | 43 | 429 | 100 | 6401 | 100 | 0.254 |
| 18.679 | 4.7465 | 43 | 103 | 24 | 1639 | 25.6 | 0.271 |
| 19.559 | 4.5349 | 43 | 49 | 11.4 | 1075 | 16.8 | 0.373 |
| 19.939 | 4.4492 | 44 | 163 | 38 | 2583 | 40.4 | 0.269 |
| 20.281 | 4.375 | 44 | 32 | 7.5 | 1094 | 17.1 | 0.581 |
| 21.04 | 4.2189 | 42 | 32 | 7.5 | 452 | 7.1 | 0.24 |
| 21.92 | 4.0515 | 37 | 92 | 21.4 | 1366 | 21.3 | 0.252 |
| 23.539 | 3.7763 | 50 | 67 | 15.6 | 599 | 9.4 | 0.152 |
| 23.921 | 3.717 | 44 | 50 | 11.7 | 1097 | 17.1 | 0.373 |
| 24.599 | 3.616 | 47 | 109 | 25.4 | 1272 | 19.9 | 0.198 |
| 25.579 | 3.4796 | 44 | 149 | 34.7 | 3200 | 50 | 0.365 |
| 26.22 | 3.396 | 53 | 91 | 21.2 | 838 | 13.1 | 0.157 |
| 27.659 | 3.2225 | 39 | 119 | 27.7 | 1890 | 29.5 | 0.27 |
| 28.421 | 3.1378 | 43 | 39 | 9.1 | 697 | 10.9 | 0.304 |
| 29.358 | 3.0397 | 40 | 36 | 8.4 | 412 | 6.4 | 0.195 |
| 30.061 | 2.9702 | 37 | 54 | 12.6 | 656 | 10.2 | 0.207 |
| 31.761 | 2.815 | 39 | 28 | 6.5 | 570 | 8.9 | 0.346 |
| 34.859 | 2.5716 | 27 | 38 | 8.9 | 559 | 8.7 | 0.25 |

### Examples of Formula III and Formula IV

### Synthesis of Int-1

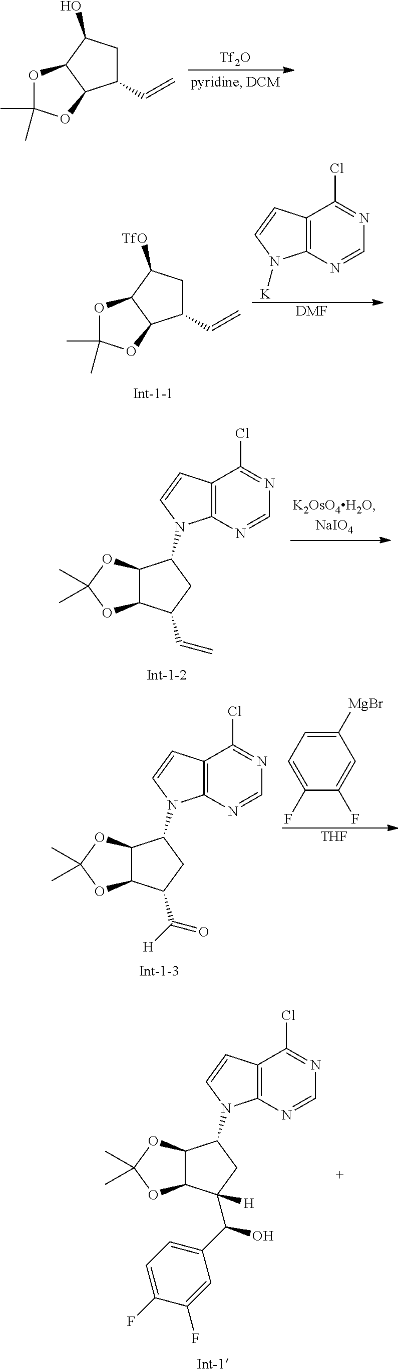

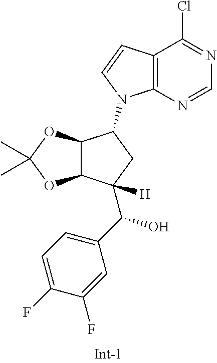 [SMILES: CC1(C)O[C@@H]2[C@@H]([C@H](O)c3ccc(F)c(F)c3)C[C@@H](n3ccc4c(Cl)ncnc43)[C@@H]2O1]

### Step 1. Synthesis of [(3aR,4S,6R, 6aR)-2,2-dimethyl-6-vinyl-4,5,6,6a-tetrahydro-3aH-cyclopenta[d][1,3]dioxol-4-yl] trifluorometh-anesulfonate (Int-1-1)

To a mixture of (3aS,4S,6R,6aR)-2,2-dimethyl-6-vinyl-4,5,6,6a-tetrahydro-3aH-cyclopenta[d][1,3]dioxol-4-ol (2000.00 mg, 10.86 mmol, prepared according to J. Org. Chem., 2004, vol. 69, 3993-3996) in DCM (20 mL) was added the pyridine (4293.55 mg, 54.28 mmol), then Tf₂O (4594.04 mg, 16.28 mmol) in DCM (10.00 mL) was added slowly to the mixture at 0° C. and the reaction was stirred for 0.5 hour at rt. TLC (PE:EA=10:1) showed the start materials was consumed completely and a new spot was on TLC. The mixture was washed with NH₄Cl solution and the organic layer was dried over Na₂SO₄ and concentrated in vacuo. The residue was purified by silica gel column (PE:EA=22:1) to give the desired product [(3aR,4S,6R, 6aR)-2,2-dimethyl-6-vinyl-4,5,6,6a-tetrahydro-3aH-cyclopenta[d][1,3]dioxol-4-yl] trifluorometh-anesulfonate (Int-1-1) (2.89 g, 9.14 mmol, 84.17% yield) and used directly in the next step.

### Step 2. Synthesis of 7-[(3aS,4R,6R,6aR)-2,2-dimethyl-6-vinyl-4,5,6,6a-tetrahydro-3aH-cyclopenta[d][1,3]dioxol-4-yl]-4-chloro-pyrrolo[2,3-d]pyrimidine (Int-1-2)

To a solution of (4-chloropyrrolo[2,3-d]pyrimidin-7-yl)potassium (2166.92 mg, 11.31 mmol) in DMF (22.5 mL) was added [(3aR,4S,6R,6aR)-2,2-dimethyl-6-vinyl-4,5,6,6a-tetrahydro-3aH-cyclopenta[d][1,3]dioxol-4-yl] trifluoromethanesulfonate (Int-1-1) (2.98 g, 9.42 mmol) in DMF (7.5 mL) dropwise at 0° C. Then the mixture was stirred at 25° C. for 16 hrs. LCMS showed Int-1-2 is the major product in the reaction mixture. The mixture was diluted by EA (300.00 mL), washed with H₂O (30.00 mL×5) and NaCl aqueous solution (50.00 mL). The combined organic layers were dried over Na₂SO₄ and concentrated in vacuo. The residue was purified by silica gel column (PE:EA=15:1) to give the desired product 7-[(3aS,4R,6R,6aR)-2,2-dimethyl-6-vinyl-4,5,6,6a-tetrahydro-3aH-cyclopenta[d][1,3]dioxol-4-yl]-4-chloro-pyrrolo[2,3-d]pyrimidine (Int-1-2) (2.00 g, 6.13 mmol, 65.09% yield) as white solid. LCMS [M+H]: 320.1.

### Step 3. Synthesis of (3aS,4R,6S,6aR)-4-(4-chloropyrrolo[2,3-d]pyrimidin-7-yl)-2,2-dimethyl-4,5,6,6a-tetrahydro-3aH-cyclopenta[d][1,3]dioxole-6-carbaldehyde (Int-1-3)

To a mixture of 7-[(3aS,4R,6R,6aR)-2,2-dimethyl-6-vinyl-4,5,6,6a-tetrahydro-3aH-cyclopenta [d][1,3]dioxol-4-yl]-4-chloro-pyrrolo[2,3-d]pyrimidine (Int-1-2) (1.6 g, 5.00 mmol) in acetone (25.0 mL), methanol (25.0 mL), water (25.0 mL) was added K₂OsO₄.2H₂O (184.3 mg, 0.5 mmol) and NaIO₄ (2140.3 mg, 10.01 mmol), then the mixture was stirred at 20° C. for 18 hrs. The mixture was filtered and the filtrate was concentrated in vacuo. The residue was dissolved in THF/H₂O (75.00 mL, v:v=2:1) and the NaIO₄ (2140.0 mg) was added. The mixture was stirred at 25° C. for 1 h. LCMS showed 39% 4 was in the mixture. The mixture was filtered and the aqueous phase was extracted with EA (50.0 mL×2). The combined organic layers were dried over Na₂SO₄, filtered and concentrated under vacuum. The crude (3aS,4R,6S,6aR)-4-(4-chloropyrrolo[2,3-d]pyrimidin-7-yl)-2,2-dimethyl-4,5,6,6a-tetrahydro-3aH-cyclopenta[d][1,3]dioxole-6-carbaldehyde (Int-1-3) (2000.00 mg, 2.75 mmol, 55.06% yield) was obtained. LCMS [M+H]: 322.1.

### Step 4. Synthesis of (S)-[(3aS,4R,6R,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]-(3,4-difluorophenyl)methanol (Int-1)

To a solution of (3aS,4R,6S,6aR)-4-(4-chloropyrrolo[2,3-d]pyrimidin-7-yl)-2,2-dimethyl-4,5,6,6a-tetrahydro-3aH-cyclopenta[d][1,3]dioxole-6-carbaldehyde (Int-1-3) (1.95 g, 2.67 mmol) in THE (20.00 mL) was added bromo-(3,4-difluorophenyl)magnesium (8.00 mL, 8.00 mmol) at 0° C. The solution was stirred at 0° C. for 1 h. LC-MS showed desired products are the major peak. The mixture was diluted by H₂O (30.00 mL) and EA (60.00 mL). EA layer was separated, washed with H₂O (30.00 mL) and NaCl aqueous solution (saturated, 30.00 mL), dried over Na₂SO₄, filtered, and concentrated under vacuum. The residue was purified by column (PE:EA=5:1 to PE:EA=3:1) to afford (R)-[(3aS,4R,6R,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]-(3,4-difluorophenyl)methanol (Int-1′) (380 mg, 0.87 mmol, 32.70% yield), and (S)-[(3aS,4R,6R,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]-(3,4-difluorophenyl)methanol (Int-1) (450 mg, 1.03 mmol, 38.7% yield).

### Synthesis of Int-2

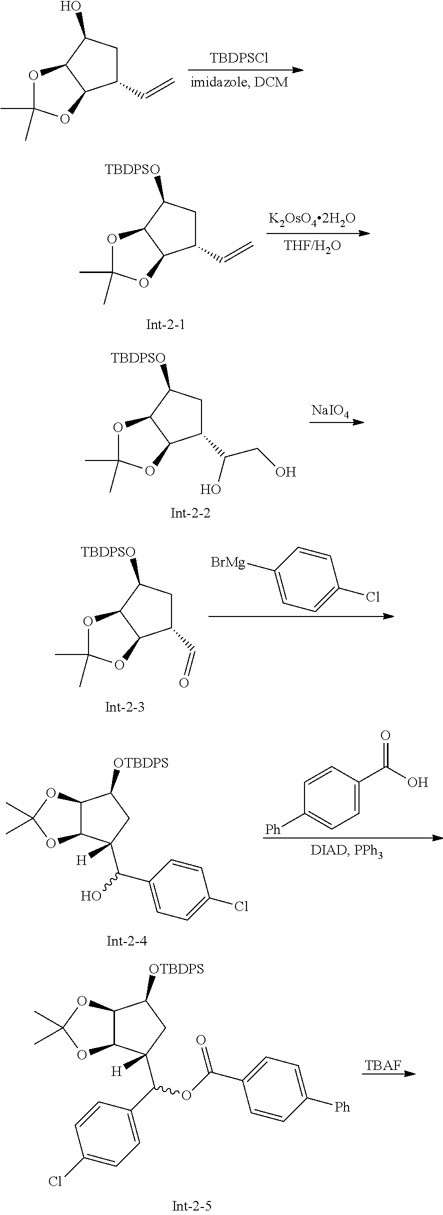

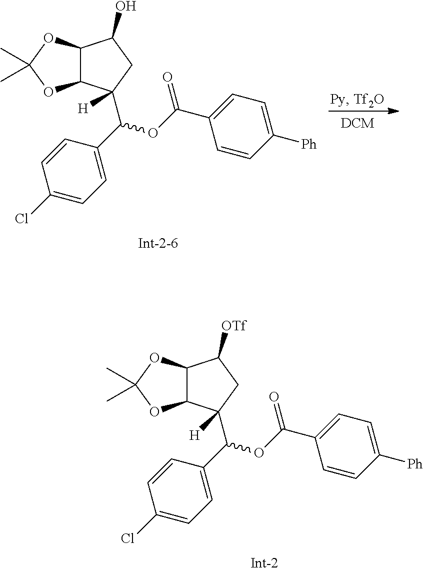

### Step 1: Synthesis of Compound Int-2-1

To a solution of (3aS,4S,6R,6aR)-2,2-dimethyl-6-vinyl-4,5,6,6a-tetrahydro-3aH-cyclopenta[d][1,3]dioxol-4-ol (2.5 g, 13.6 mmol, prepared according to J. Org. Chem., 2004, vol. 69, 3993-3996) in DCM (10 mL) was added the imidazole (1.85 g, 27.1 mmol) and TBDPSCl (5.6 g, 20 mmol), then the mixture was stirred at 25° C. for 2 h. TLC (PE:EtOAc=10:1) showed the starting materials was consumed completed and a new spot was detected. The reaction was concentrated to dryness and the residue was diluted with EtOAc (100 ml), which was washed with water (50 ml×2) and brine (40 ml). The combined organic layers were dried over Na₂SO₄ before concentration to dryness. The residue was purified by flash column chromatography (PE:EtOAc=40:1 to 20:1) to afford Int-2-1 (5.6 g, 13.25 mmol, 97.6%) as a colorless oil. ¹H NMR (400 MHz, CDCl₃): δ 7.76-7.71 (m, 4H), 7.43-7.35 (m, 6H), 5.57-5.49 (m, 1H), 4.83 (d, 1H), 4.74 (d, 1H), 4.27-4.21 (m, 2H), 4.04-3.99 (m, 1H), 2.56 (t, 1H), 2.08-2.00 (m, 1H), 1.59-1.52 (m, 1H), 1.59 (s, 3H), 1.30 (s, 3H), 1.07 (s, 9H).

### Step 2: Synthesis of Compound Int-2-2

To a solution of compound Int-2-1 (5.6 g, 13 mmol) in THE (100 mL) and H₂O (50 mL) was added NMO (2.3 g, 20 mmol) and K₂OsO₄.2H₂O (488 mg, 1.33 mmol), then the reaction was stirred at 25° C. overnight. TLC (PE:EtOAc=1:1) showed the starting materials was consumed completely and a new spot was detected. EtOAc (20 mL) was added to the mixture and the organic layer was washed with saturated Na₂SO₃ solution (2 mL) and brine (10 mL). The organic layer was dried over Na₂SO₄, concentrated in vacuo to afford a residue. The residue was purified by silica gel chromatography eluted with petroleum ether:EtOAc=3:1 to 1:1 to afford Int-2-2 (4.6 g, 9.44 mmol, 71.25%) as a colorless oil. LCMS [M+23] 479.2; ¹H NMR (400 MHz, CDCl₃): δ 7.76-7.71 (m, 4H), 7.44-7.35 (m, 6H), 4.28-4.11 (m, 3H), 3.50-3.33 (m, 3H), 2.10-2.04 (m, 1H), 1.94-1.88 (m, 1H), 1.58-1.52 (m, 1H), 1.54 (s, 3H), 1.31 (s, 3H), 1.08 (s, 9H).

### Step 3: Synthesis of Compound Int-2-3

To a solution of compound Int-2-2 (4.6 g, 10. mmol) in THE (50 mL) and water (25 mL) was added NaIO₄ (6.46 g, 30.2 mmol), then the mixture was stirred at 25° C. for 2 h. TLC (PE:EtOAc=3:1) showed the starting materials was consumed and a new spot was detected. The mixture was poured into EtOAc (100 ml) and the organic layer was washed with water (50 ml×2) and brine (50 ml). The organic layer was dried over Na₂SO₄ and concentrated in vacuo to give the crude Int-2-3 (4.0 g, 9.42 mmol, 93.52%) as a colorless oil, which was used in the next step without further purification. ¹H NMR (400 MHz, CDCl₃): δ 9.47 (s, 1H), 7.74-7.68 (m, 4H), 7.45-7.34 (m, 6H), 4.70 (d, 1H), 4.18-4.13 (m, 1H), 3.81-3.76 (m, 3H), 2.77 (d, 1H), 3.50-3.33 (m, 3H), 2.16-2.04 (m, 1H), 1.92-1.88 (m, 1H), 1.53 (s, 3H), 1.30 (s, 3H), 1.07 (s, 9H).

### Step 4: Synthesis of Compound Int-2-4

To a solution of compound Int-2-3 (4.0 g, 9.42 mmol) in dry THE (20 mL) was added Bromo (4-chlorophenyl)magnesium (1M in THF, 47.1 mL, 47.1 mmol), then the reaction was stirred at 25° C. for 1 h. TLC (PE:EtOAc=3:1) showed the starting material was consumed completely and a new spot was detected. H₂O (1 ml) was added to quench the reaction and the mixture was diluted with EtOAc (50 ml), washed with H₂O (40 ml×2) and brine (30 ml×2). The organic layer was separated and dried over Na₂SO₄ before concentration to dryness. The residue was purified by silica gel chromatography eluted with petroleum ether:EtOAc=5:1 to 3:1 to afford Int-2-4 (3.5 g, 6.52 mmol, 69.17%) as a white solid. ¹H NMR (400 MHz, CDCl₃): δ 7.75-7.68 (m, 4H), 7.46-7.30 (m, 6H), 7.24-6.97 (m, 4H), 4.63-4.55 (m, 1H), 4.38-4.03 (m, 3H), 2.25-2.23 (m, 1H), 1.83-1.75 (m, 2H), 1.54 (s, 3H, two peaks from epimers), 1.31 (s, 3H, two peaks from epimers), 1.03 (s, 9H, two peaks from epimers).

### Step 5: Synthesis of Compound Int-2-5

To a solution of compound Int-2-4 (3.5 g, 6.52 mmol) in toluene (100 mL) was added PPh₃ (2.56 g, 9.77 mmol), 4-phenylbenzoic acid (1.94 g, 9.77 mmol), DIAD (1.98 g, 9.77 mmol) at 0° C., then the mixture was stirred at 20° C. for 2 h. TLC (PE:EtOAc=10:1) showed the starting materials was consumed completely and a new spot was detected. The mixture was concentrated in vacuo and the residue was purified by silica gel chromatography eluted with petroleum ether:EtOAc=30:1 to 20:1 to afford Int-2-5 (4.60 g, 6.41 mmol, 98.4%) as a white solid. ¹H NMR (400 MHz, CDCl₃): δ 8.25-7.01 (m, 23H), 5.85-5.37 (m, 1H), 4.45-4.27 (m, 2H), 4.14-4.09 (m, 1H), 2.54-2.49 (m, 1H), 2.04-1.97 (m, 1H), 1.67-1.63 (m, 1H), 1.56 (s, 3H, two peaks from epimers), 1.28 (s, 3H, two peaks from epimers), 1.04 (s, 9H, two peaks from epimers).

### Step 6: Synthesis of Compound Int-2-6

To a solution of compound Int-2-5 (4.60 g, 6.41 mmol) in THE (20 mL) was added tetrabutylammonium fluoride (1 μM in THF, 32 mL, 32 mmol), then the mixture was stirred at 25° C. overnight. TLC (PE:EtOAc=3:1) showed the starting materials was almost consumed completely. The solution was concentrated in vacuo and purified by silica gel chromatography eluted with petroleum ether:EtOAc=10:1 to 5:1 to afford Int-2-6 (1.60 g, 3.34 mmol, 52.1%) as a white solid. ¹H NMR (400 MHz, CDCl₃): δ 8.13-7.26 (m, 13H), 6.11-5.73 (m, 1H), 4.63-4.46 (m, 2H), 4.29-4.11 (m, 1H), 2.78-2.75 (m, 1H), 2.50-2.39 (m, 1H), 2.04-1.91 (m, 1H), 1.53 (s, 3H, two peaks from epimers), 1.33 (s, 3H, two peaks from epimers).

### Step 7: Synthesis of Compound Int-2

To a solution of compound Int-2-6 (1.60 g, 3.34 mmol) in DCM (20 mL) was added the pyridine (1.4 mL, 16.7 mmol), then Tf₂O (1.41 g, 5.01 mmol) was added slowly to the mixture and stirred at rt for 2 h. TLC (PE:EtOAc=10:1) showed the starting materials was consumed and a new spot was detected. The mixture was purified by silica gel chromatography eluted with petroleum ether:EtOAc=20:1 to 10:1 to give the desired product Int-2 (1.80 g, 2.95 mmol, 88.2%) as a white solid. ¹H NMR (400 MHz, CDCl₃): δ 8.13-7.28 (m, 13H), 6.16-5.82 (m, 1H), 5.34-5.07 (m, 1H), 4.72-4.54 (m, 2H), 2.84-2.82 (m, 1H), 2.34-2.12 (m, 2H), 1.53 (s, 3H, two peaks from epimers), 1.31 (s, 3H, two peaks from epimers).

### Synthesis of Int-3

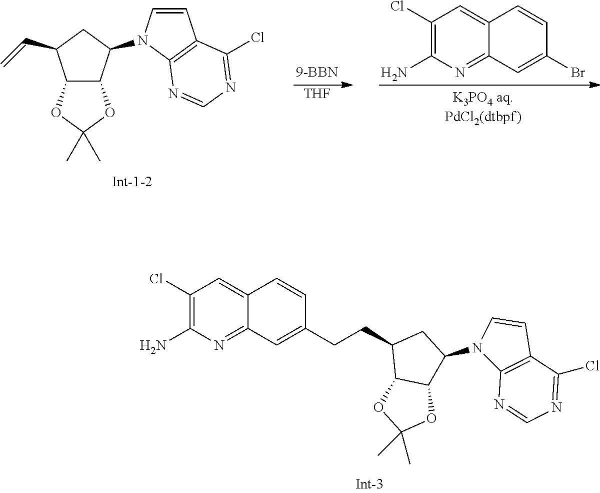

A solution of compound Int-1-2 (0.65 g, 2.03 mmol, 1 eq.) and 9-BBN (0.5 M, 12.20 mL, 3 eq.) was stirred at 75° C. for 45 min. LC-MS showed compound Int-1-2 was consumed completely. To the reaction solution was added K₃PO₄ (1.24 g, 5.82 mmol, 3.04 eq.), 7-bromo-3-chloro-quinolin-2-amine (493.03 mg, 1.91 mmol, 1 eq.), H₂O (2.4 mL), THE (12 mL) and ditert-butyl(cyclopentyl)phosphane; dichloropalladium; iron (124.78 mg, 191.46 umol, 0.1 eq.). The mixture was sealed and stirred at 75° C. for 12 h. LC-MS showed several new peaks and desired compound was detected. The mixture was diluted with H₂O (50 mL), and extracted with EtOAc (50 mL*3). The combined organic phase was washed with saturated NaCl (50 mL*2), dried with anhydrous Na₂SO₄, and concentrated in vacuo. The residue was purified by column chromatography (SiO₂, Petroleum ether/Ethyl acetate=50:1 to 1:1). Compound Int-3 (0.2 g, 264.85 umol, 13.83% yield, 66% LCMS purity) was obtained as a yellow solid checked. LCMS: (M+H⁺): 498.1.

### Synthesis of Int-4

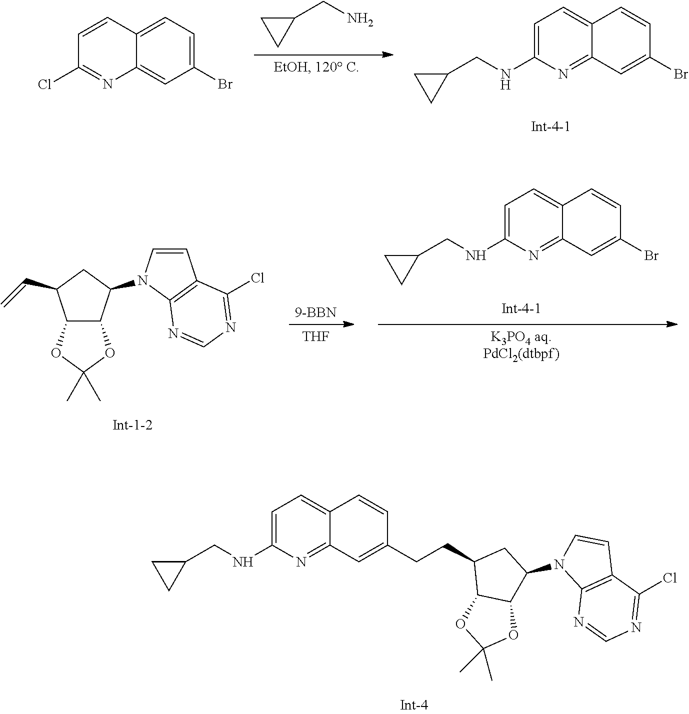

### Step 1. Synthesis of Int-4-1

A mixture of 7-bromo-2-chloro-quinoline (2 g, 8.25 mmol, 1 eq.) and cyclopropylmethanamine (6.15 g, 86.53 mmol, 6 mL, 10.49 eq.) in EtOH (10 mL) was stirred at 120° C. for 12 hrs. TLC showed the reaction was complete. The mixture was concentrated. The residue was purified by column chromatography (SiO₂, Petroleum ether/Ethyl acetate=10:1 to 1:1). Int-4-1 (2.2 g, 7.63 mmol, 92.52% yield, 96.127% purity) was obtained as a yellow solid. ¹H NMR (400 MHz, CHLOROFORM-d) δ=7.63 (d, J=1.8 Hz, 1H), 7.53 (d, J=8.9 Hz, 1H), 7.20 (d, J=8.4 Hz, 1H), 7.09-7.05 (m, 1H), 6.40 (d, J=8.9 Hz, 1H), 4.66 (br s, 1H), 3.13 (dd, J=5.3, 7.1 Hz, 2H), 0.91 (tquin, J=4.8, 7.5 Hz, 1H), 0.41-0.31 (m, 2H), 0.13-0.03 (m, 2H); LCMS: (M+H⁺): 276.9, 278.9; LCMS purity: 96.12%; TLC (Petroleum ether:Ethyl acetate=3:1) R_{f}=0.60.

### Step 2. Synthesis of Int-4

A mixture of Int-1-2 (1.5 g, 4.69 mmol, 1 eq.) in 9-BBN (0.5 M, 30 mL, 3.20 eq.) was stirred at 75° C. for 1 hr. LCMS showed the starting material was consumed. It was cooled to 25° C. The solution was used directly in the next step. To the above solution in 30 mL THE was added 7-bromo-N-(cyclopropylmethyl)quinolin-2-amine (Int-4-1) (1.56 g, 5.61 mmol, 1.2 eq.), K₃PO₄ (3.02 g, 14.22 mmol, 3.04 eq.) and ditert-butyl(cyclopentyl)phosphane; dichloropalladium; iron (0.2 g, 306.87 umol, 0.066 eq.), H₂O (7.8 mL) and THE (60 mL). The mixture was stirred at 75° C. for 12 hr under N₂. LCMS showed the reaction was completed. It was cooled to 20° C. and concentrated at 45° C. The residue was purified by column chromatography (SiO₂, Petroleum ether/Ethyl acetate=15/1 to 3/1). Int-4 (0.5 g, 965.16 umol, 20.64% yield) was obtained as a brown oil. ¹H NMR (400 MHz, CHLOROFORM-d) δ=8.56 (s, 1H), 7.70 (d, J=8.8 Hz, 1H), 7.48-7.38 (m, 2H), 7.21 (d, J=3.7 Hz, 1H), 7.01 (d, J=8.2 Hz, 1H), 6.57-6.49 (m, 2H), 4.98-4.81 (m, 2H), 4.75 (br s, 1H), 4.50-4.40 (m, 1H), 3.34-3.18 (m, 2H), 2.85-2.70 (m, 2H), 2.42 (td, J=6.0, 11.8 Hz, 1H), 2.23-2.13 (m, 1H), 2.03-1.94 (m, 1H), 1.86-1.73 (m, 2H), 1.50 (s, 3H), 1.24 (s, 3H), 1.11-1.00 (m, 1H), 0.54-0.45 (m, 2H), 0.23 (q, J=4.8 Hz, 2H); LCMS: (M+H+): 518.0; TLC (Petroleum ether:Ethyl acetate=2:1) Rf=0.24.

### Example 25-B. 7-[(1R,2S,3R,4R)-4-[(S)-(4-chlorophenyl)-hydroxy-methyl]-2,3-dihydroxy-cyclopentyl]-1H-pyrrolo[2,3-d]pyrimidin-4-one hydrazone hydrochloride (Ex. 25-B)

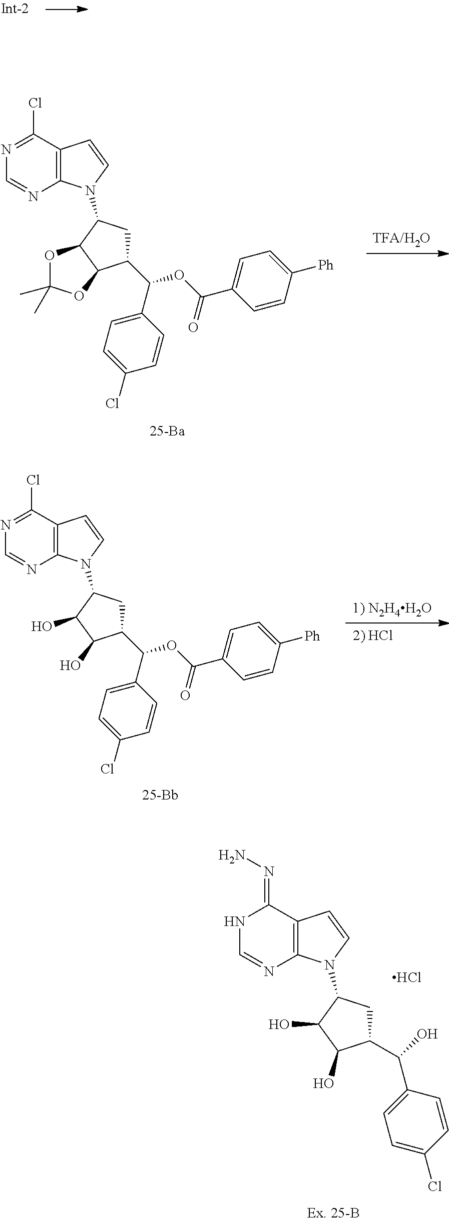

### a) Synthesis of [(S)-[(3aS,4R,6R,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]-(4-chlorophenyl)methyl] 4-phenylbenzoate (25-Ba)

Compound 25-Ba was prepared similarly to that of Int-1-2.

### b) Synthesis of [(S)-(4-chlorophenyl)-[(1S,2R,3S,4R)-4-(4-chloropyrrolo[2,3-d]pyrimidin-7-yl)-2,3-dihydroxy-cyclopentyl]methyl] 4-phenylbenzoate (25-Bb)

To a solution of [(S)-[(3aS,4R,6R,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]-(4-chlorophenyl)methyl] 4-phenylbenzoate (25-Ba) (210.0 mg, 0.34 mmol) in THE (2.0 mL), TFA (1.0 mL, 13.51 mmol) in water (1.0 mL, 55.49 mmol) was added. The mixture was stirred at 25° C. for 4 hrs. LCMS showed 83% desired product was detected in 254 nm. The solvent was removed, and the mixture was extracted with EA (30.0 mL×3). The combined organic layers were dried over Na₂SO₄ and concentrated in vacuum to give the crude [(S)-(4-chlorophenyl)-[(1S,2R,3S,4R)-4-(4-chloropyrrolo[2,3-d]pyrimidin-7-yl)-2,3-dihydroxy-cyclopentyl]methyl] 4-phenylbenzoate (25-Bb) (130.0 mg, 0.22 mmol, 66.12% yield) as yellow solid which was used in the next step directly. LCMS [M+H]: 574.1.

### c) Synthesis of 7-[(1R,2S,3R,4R)-4-[(S)-(4-chlorophenyl)-hydr oxy-methyl]-2,3-dihydroxy-cyclopentyl]-1H-pyrrolo[2,3-d]pyrimidin-4-one hydrazone hydro chloride (Ex. 25-B)

To a solution of [(S)-(4-chlorophenyl)-[(1S,2R,3S,4R)-4-(4-chloropyrrolo[2,3-d]pyrimidin-7-yl)-2,3-dihydroxy-cyclopentyl]methyl] 4-phenylbenzoate (25-Bb) (40.0 mg, 0.06 mmol) in ethanol (1.0 mL), hydrazine hydrate (2.5 mL, 51.23 mmol) was added. The mixture was stirred at 25° C. for 16 hrs. LCMS (SYZ001-50-R1) showed the reaction was completed. The mixture was adjusted to pH≈2 with 4 M HCl aqueous solution. The mixture was purified by prep-HPLC eluting with CH₃CN/H₂O (0.1% TFA contained) from 5/95 to 95/5 to give 7-[(1R,2S,3R,4R)-4-[(S)-(4-chlorophenyl)-hydr oxy-methyl]-2,3-dihydroxy-cyclopentyl]-1H-pyrrolo[2,3-d]pyrimidin-4-one hydrazone hydro chloride (Ex. 25-B) (3.7 mg, 0.01 mmol, 13.89% yield) as a white solid. LCMS [M+H]: 390.1. ¹H NMR (400 M Hz, CD₃OD): δ 8.19 (s, 1H), 7.51 (s, 1H), 7.32-7.43 (m, 4H), 6.79 (s, 1H), 5.04-5.11 (m, 1H), 4.68 (d, J=6.8 Hz, 1H), 4.40 (m, 1H), 4.16 (m, 1H), 2.40-2.44 (m, 1H), 2.11-2.19 (m, 1H), 1.73-1.81 (m, 1H).

### Example 28-B. 7-[(1R,2S,3R,4R)-4-[(S)-(4-chlorophenyl)-hydroxy-methyl]-2,3-dihydroxy-cyclopentyl]-5-fluoro-H-pyrrolo[2,3-d]pyrimidin-4-one hydrazone hydrochloride (Ex. 28-B)

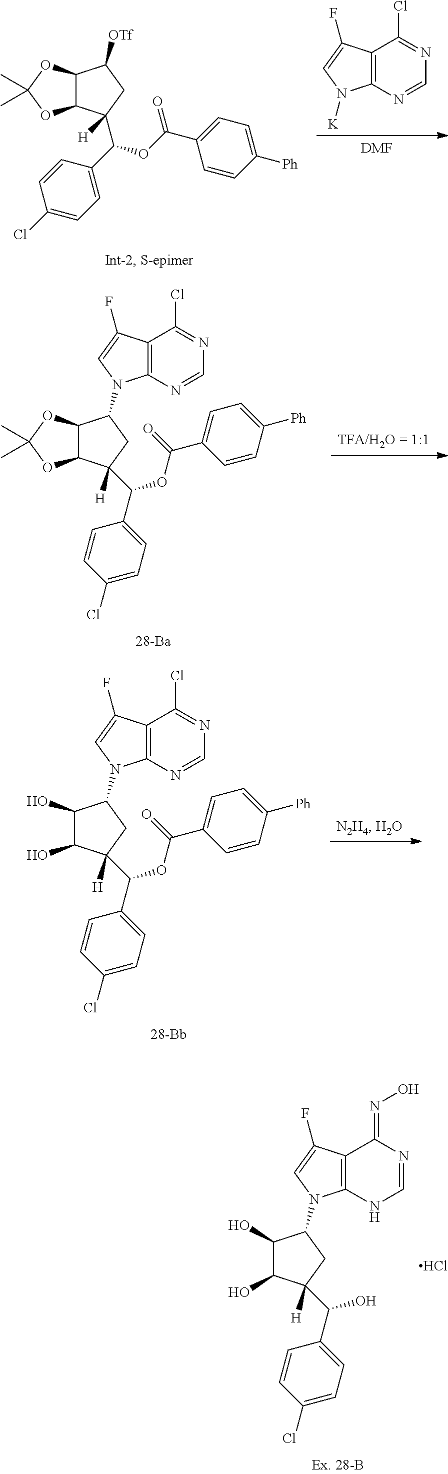

### a) Synthesis of 7-[(3aS,4S,6R,6aR)-6-[(S)-(4-chlorophenyl)-[(4-phenylphenyl)methoxy]methyl]-2,2-dimethyl-4, 5,6,6a-tetrahydro-3 aH-cyclopenta[d][1,3]dioxol-4-yl]-4-chloro-5-fluoro-pyrrolo[2,3-d]pyrimidine (28-Ba)

To a solution of [(3aR,4S,6R,6aR)-6-[(S)-(4-chlorophenyl)-[(4-phenylphenyl)methoxy]methyl]-2,2-dimethyl-4, 5,6,6a-tetrahydro-3 aH-cyclopenta[d][1,3]dioxol-4-yl]trifluoromethanesulfonate (Int-2, S-epimer) (199.4 mg, 0.33 mmol) in DMF (10.0 mL) was added (4-chloro-5-fluoropyrrolo[2,3-d]pyrimidin-7-yl)potassium (70.0 mg, 0.33 mmol), then stirred at 25° C. for 4 hrs. After the reaction was complete, the reaction mixture was concentrated in vacuum, taken into water, and extracted with EA (20.0 mL). The organic layer was concentrated in vacuum to give crude 7-[(3 aS,4 S,6R,6aR)-6-[(S)-(4-chlorophenyl)-[(4-phenylphenyl)methoxy]methyl]-2,2-dimethyl-4, 5,6,6a-tetrahydro-3 aH-cyclopenta[d][1,3]dioxol-4-yl]-4-chloro-5-fluoro-pyrrolo[2,3-d]pyrimidine (28-Ba) (200.0 mg, 0.27 mmol, 82.3% o yield) which was used in the next step directly. LCMS [M+H]: 632.2.

### b) Synthesis of [(S)-[(1S,2R,3S,4R)-4-(4-chloro-5-fluoro-pyrrolo[2,3-d]pyrimidin-7-yl)-2,3-dihydroxy-cyclopentyl]-(4-chlorophenyl)methyl] 4-phenylbenzoate (28-Bb)

To a solution of [(S)-[(3aS,4R,6R,6aR)-4-(4-chloro-5-fluoro-pyrrolo[2,3-d]pyrimidin-7-yl)-2,2-dimethyl-4, 5,6,6a-tetrahydro-3 aH-cyclopenta[d][1,3]dioxol-6-yl]-(4-chlorophenyl)methyl] 4-phenylbenzoate (28-Ba) (200.0 mg, 0.27 mmol) in THE (3.0 mL) and water (25.0 mL) was added TFA (5.0 mL, 67.53 mmol). The reaction mixture was stirred at 20° C. for 12 hrs. After the reaction was complete, the reaction mixture was concentrated in vacuum and extracted with EA (30.0 mL). The organic layer was dried over Na₂SO₄, filtered and concentrated in vacuum to give the crude [(S)-[(1S,2R,3S,4R)-4-(4-chloro-5-fluoro-pyrrolo[2,3-d]pyrimidin-7-yl)-2,3-dihydroxy-cyclopentyl]-(4-chlorophenyl)methyl] 4-phenylbenzoate (28-Bb) (70.0 mg, 0.07 mmol) as yellow oil which was used in the next step directly. LCMS [M+H]: 592.3.

### c) Synthesis of 7-[(1R,2S,3R,4R)-4-[(S)-(4-chlorophenyl)-hydroxy-methyl]-2,3-dihydroxy-cyclopentyl]-5-fluoro-H-pyrrolo[2,3-d]pyrimidin-4-one hydrazone hydrochloride (Ex. 28-B)

To a solution of [(S)-[(1S,2R,3S,4R)-4-(4-chloro-5-fluoro-pyrrolo[2,3-d]pyrimidin-7-yl)-2,3-dihydroxy-cyclopentyl]-(4-chlorophenyl)methyl] 4-phenylbenzoate (70.0 mg, 0.07 mmol) in ethanol (3.0 mL) was added N₂H₄.H₂O (3.0 mL, 61.73 mmol). The mixture was stirred at 20° C. for 2 hrs. After the reaction was complete, the mixture was concentrated in vacuum to give the residue, which was purified by prep-HPLC (0.1% TFA, 10-40% MeCN/H₂O) to afford the pure product as TFA salt. HCl (0.5 mL, 1 μM) was added to the solution and lyophilized to afford 7-[(1R,2S,3R,4R)-4-[(S)-(4-chlorophenyl)-hydroxy-methyl]-2,3-dihydroxy-cyclopentyl]-5-fluoro-H-pyrrolo[2,3-d]pyrimidin-4-one hydrazone hydrochloride (Ex. 28-B) (9.5 mg, 0.02 mmol, 30.2% yield) as yellow solid. LCMS [M+H]: 409.3. ¹H NMR (400 MHz, CD₃OD-d4): δ 8.22 (s, 1H), 7.46-7.33 (m, 5H), 5.13-5.14 (m, 1H), 4.68 (d, J=6.8 Hz, 1H), 4.37 (m, 1H), 4.18 (d, J=3.2 Hz, 1H), 2.43 (m, 1H), 2.13 (m, 1H), 1.73 (m, 1H).

### Example 37-B. 7-[(1R,2S,3R,4R)-4-[(S)-(3,4-difluorophenyl)-hydroxy-methyl]-2,3-dihydroxy-cyclopentyl]-1H-pyrrolo[2,3-d]pyrimidin-4-one hydrazone hydrochloride (Ex. 37-B)

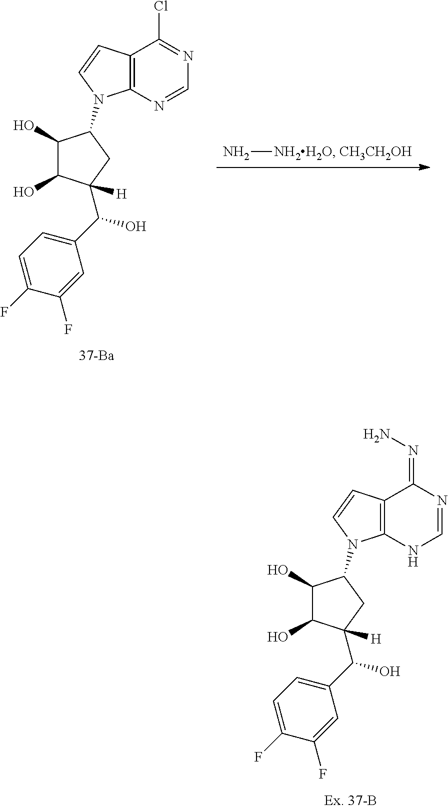

### a) Synthesis of (1R,2S,3R,5R)-3-(4-chloropyrrolo[2,3-d]pyrimidin-7-yl)-5-[(S)-(3,4-difluorophen-yl)-hydroxy-methyl]cyclopentane-1,2-diol (37-Ba)

Compound 37-Ba was prepared by treated Int-1 with either TFA/H₂O or aq. HCl in methanol.

### b) Synthesis of 7-[(1R,2S,3R,4R)-4-[(S)-(3,4-difluorophenyl)-hydroxy-methyl]-2,3-dihydroxy-cyclopentyl]-1H-pyrrolo[2,3-d]pyrimidin-4-one hydrazone hydrochloride (Ex. 37-B)

To a solution of (1R,2S,3R,5R)-3-(4-chloropyrrolo[2,3-d]pyrimidin-7-yl)-5-[(S)-(3,4-difluorophen-yl)-hydroxy-methyl]cyclopentane-1,2-diol (37-Ba) (79.00 mg, 0.20 mmol) in ethanol (3.00 mL) was added hydrazine hydrate (0.10 mL, 2.10 mmol). The mixture was stirred at 20° C. for 1 h. LC-MS showed the reaction was not complete, and additional hydrazine hydrate (0.20 mL, 4.12 mmol) was added. The mixture was stirred for another 1 h at 20° C. and LC-MS showed 55% product in the reaction mixture. Another batch of hydrazine hydrate (0.10 mL, 2.06 mmol) was added, and the mixture was stirred for another 1 h at 20° C. LC-MS showed the desired product is the major peak. The mixture was purified by prep-HPLC (0.1% TFA, H₂O:CH₃CN from 90:10 to 5:95), then 0.05 mL of conc. HCl was added. The mixture was lyophilized to afford 7-[(1R,2S,3R,4R)-4-[(S)-(3,4-difluorophenyl)-hydroxy-methyl]-2,3-dihydroxy-cyclopentyl]-1H-pyrrolo[2,3-d]pyrimidin-4-one hydrazone hydrochloride (Ex. 37-B) (30.6 mg, 0.07 mmol, 35.56% yield) as white solid. LCMS [M+H]: 392.3. ¹HNMR (DMSO-d6, 400 MHz): δ 10.99 (b, 1H), 8.24 (s, 1H), 7.61-7.62 (m, 1H), 7.34-7.44 (m, 2H), 7.24-7.25 (m, 1H), 6.91-6.92 (m, 1H), 4.92-4.99 (m, 1H), 4.56-4.57 (m, 1H), 4.19-4.23 (m, 1H), 3.92-3.93 (m, 1H), 2.23-2.25 (m, 1H), 1.97-2.02 (m, 1H), 1.54-1.60 (m, 1H).

### Example 49-B. (1S,2R,3R,5R)-3-[(S)-(4-chlorophenyl)-hydroxy-methyl]-5-[(4Z)-4-methoxyimino-1H-pyrrolo[2,3-d]pyrimidin-7-yl]cyclopentane-1,2-diol hydrochloride (Ex. 49-B)

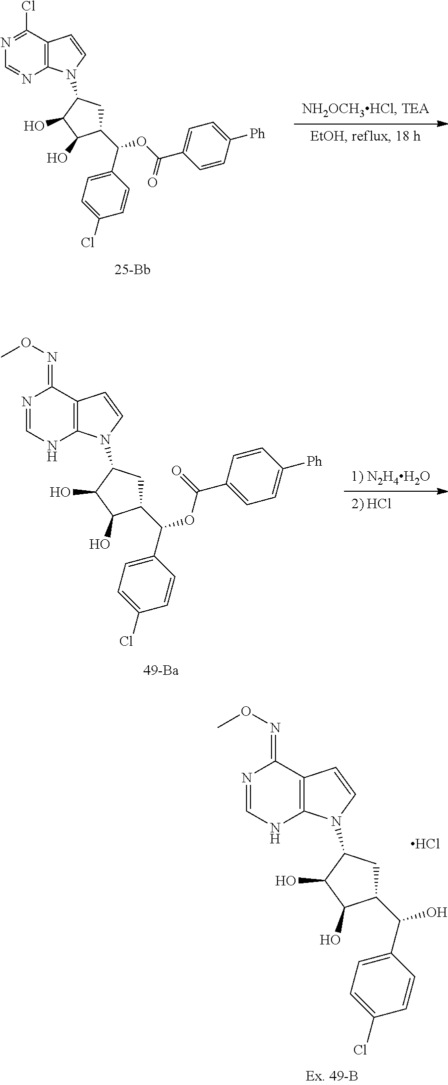

### a) Synthesis of [(S)-(4-chlorophenyl)-[(1S,2R,3S,4R)-2,3-dihydroxy-4-[(4E)-4-methoxyimino-1H-pyrrolo[2,3-d]pyrimidin-7-yl]cyclopentyl]methyl] 4-phenylbenzoate (49-Ba)

To a solution of [(S)-(4-chlorophenyl)-[(1S,2R,3S,4R)-4-(4-chloropyrrolo[2,3-d]pyrimidin-7-yl)-2,3-dihydroxy-cyclopentyl]methyl] 4-phenylbenzoate (25-Bb) (20.0 mg, 0.03 mmol) in ethanol (10.0 mL) was added the TEA (5.0 mL, 0.03 mmol) and NH₂OCH₃.HCl (90.0 mg, 1.08 mmol) in a seal vessel. The mixture was stirred at 90° C. for 16 hrs. LCMS showed the starting material was almost consumed and the desired product was detected. The mixture was concentrated in vacuum and the residue was extracted with EA (20.0 mL×3) and the organic layers were concentrated to give the crude product [(S)-(4-chlorophenyl)-[(1S,2R,3S,4R)-2,3-dihydroxy-4-[(4E)-4-methoxyimino-1H-pyrrolo[2,3-d]pyrimidin-7-yl]cyclopentyl]methyl] 4-phenylbenzoate (49-Ba) (22.0 mg, 0.02 mmol, 73.95% yield) and the crude product was used directly for the next steps. LCMS [M+H]: 585.2.

### b) Synthesis of (1S,2R,3R,5R)-3-[(S)-(4-chlorophenyl)-hydroxy-methyl]-5-[(4Z)-4-methoxyimino-1H-pyrrolo[2,3-d]pyrimidin-7-yl]cyclopentane-1,2-diol hydrochloride (Ex. 49-B)

To a solution of [(S)-(4-chlorophenyl)-[(1S,2R,3S,4R)-2,3-dihydroxy-4-[(4Z)-4-methoxyimino-1H-pyrrolo[2,3-d]pyrimidin-7-yl]cyclopentyl]methyl] 4-phenylbenzoate (49-Ba) (0.04 mL, 0.02 mmol) in ethanol (1.0 mL), hydrazine hydrate (0.2 mL, 4.12 mmol) was added, and the mixture was stirred at 25° C. for 16 hrs. LCMS showed the starting material was almost consumed. The mixture was purified by prep-HPLC eluting with CH₃CN/H₂O (0.1% TFA contained) from 5/95 to 95/5 to give the desired TFA salt as white solid. The solid was dissolved in 1 M HCl aq. (3.0 mL) and lyophilized to give (1S,2R,3R,5R)-3-[(S)-(4-chlorophenyl)-hydroxy-methyl]-5-[(4Z)-4-methoxyimino-1H-pyrrolo[2,3-d]pyrimidin-7-yl]cyclopentane-1,2-diol hydrochloride (Ex. 49-B) (2.8 mg, 0.0057 mmol, 29.13% yield) as white solid. LCMS [M+H]: 405.1. ¹H NMR (400 M Hz, CD₃OD-d4): δ 8.12 (s, 1H), 7.33-7.44 (m, 5H), 6.67 (d, J=3.6 Hz, 1H), 5.00-5.07 (m, 1H), 4.68 (d, J=2.8 Hz, 1H), 4.37-4.41 (m, 1H), 4.14-4.17 (m, 1H), 3.92 (s, 3H), 2.40-2.46 (m, 1H), 2.11-2.19 (m, 1H), 1.72-1.18 (m, 1H).

### Example 50-B. 7-[(1R,2S,3R,4R)-4-[(S)-(4-chlorophenyl)-hydroxy-methyl]-2,3-dihydroxy-cyclopentyl]-1H-pyrrolo[2,3-d]pyrimidin-4-one oxime hydrochloride (Ex. 50-B)

Example 50-B was prepared similarly to that of Ex. 49-B. LCMS [M+H]: 391.1. ¹H NMR (400 MHz, DMSO-d6) δ 8.87 (br, 2H), 8.23 (s, 1H), 7.62 (d, J=3.6 Hz, 1H), 7.42-7.36 (m, 4 H), 6.92 (d, J=3.6 Hz, 1H), 4.95 (m, 1H), 4.55 (d, J=6.8 Hz, 1H), 4.22 (m, 1H), 3.94 (d, J=4.8 Hz, 1H), 2.24-2.51 (m, 1H), 2.01-1.96 (m, 1H), 1.59-1.55 (m, 1H).

### Example 79-B. (1S,2R,3S,5R)-3-(2-(2-((cyclopropylmethyl)amino)quinolin-7-yl)ethyl)-5-(4-(1-methylhydrazineyl)-7H-pyrrolo[2,3-d]pyrimidin-7-yl)cyclopentane-1,2-diol (79-B)

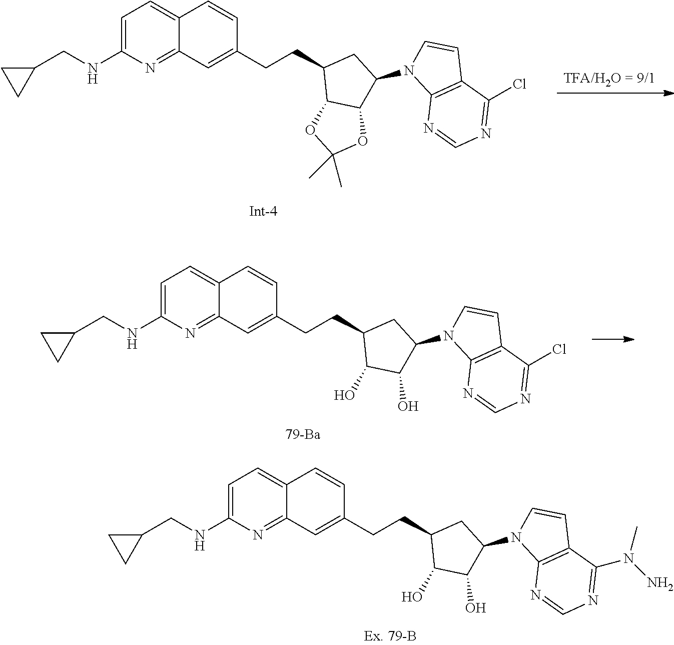

### a) Synthesis of Compound 79-Ba

A mixture of compound 1 (0.035 g, 67.56 umol, 1 eq.) in TFA (2 mL) and H₂O (0.22 mL) was stirred at 20° C. for 10 min. LCMS showed the reaction was completed, and the desired product was detected. The mixture was concentrated at 25° C. directly. The crude product compound 79-Ba (0.03 g, crude) was used into the next step without further purification (as a brown oil). LCMS: (M+H⁺): 478.2

### b) Synthesis of (1S,2R,3S,5R)-3-(2-(2-((cyclopropylmethyl)amino)quinolin-7-yl)ethyl)-5-(4-(1-methylhydrazineyl)-7H-pyrrolo[2,3-d]pyrimidin-7-yl)cyclopentane-1,2-diol (79-B)

To a solution of compound 2 (0.03 g, 62.76 umol, 1 eq.) in dioxane (4 mL) was added methylhydrazine (291.65 mg, 2.53 mmol, 333 uL, 40.3 eq.). The mixture was stirred at 100° C. for 12 hr. LCMS showed the reaction was completed. It was concentrated to give the crude product. It was purified by prep-HPLC (HCl condition) to give the product. Ex. 79-B (14 mg, 27.10 umol, 43.19% yield, 94.4% purity) was obtained as a colorless oil. 1H NMR (400 MHz, DMSO-d₆) δ=12.85 (br s, 1H), 9.63 (br s, 1H), 7.89 (s, 1H), 7.85 (br d, J=9.3 Hz, 1H), 7.79 (br s, 1H), 7.42 (br d, J=7.1 Hz, 2H), 7.00 (br d, J=7.5 Hz, 1H), 6.82 (br d, J=9.0 Hz, 1H), 6.75-6.65 (m, 1H), 4.67-4.54 (m, 1H), 3.86 (dd, J=5.8, 8.3 Hz, 1H), 2.56-2.32 (m, 2H), 2.02-1.84 (m, 1H), 1.66-1.46 (m, 2H), 1.45-1.28 (m, 1H), 1.23-1.07 (m, 1H), 0.82 (br s, 1H), 0.20 (br d, J=7.1 Hz, 2H), 0.01 (br d, J=4.2 Hz, 2H); ¹H NMR (400 MHz, DMSO-d₆+D₂O) δ=7.96-7.80 (m, 2H), 7.57 (br s, 1H), 7.45 (br d, J=8.2 Hz, 1H), 7.39 (br d, J=3.3 Hz, 1H), 7.02 (br d, J=8.2 Hz, 1H), 6.81-6.67 (m, 2H), 4.67-4.54 (m, 1H), 3.86 (dd, J=6.0, 8.4 Hz, 1H), 3.44-3.38 (m, 2H), 3.10 (br d, J=6.6 Hz, 2H), 2.55-2.35 (m, 2H), 2.02-1.85 (m, 1H), 1.68-1.47 (m, 2H), 1.46-1.31 (m, 1H), 1.27-1.11 (m, 1H), 0.82 (br s, 1H), 0.23 (br d, J=7.1 Hz, 2H), 0.01 (br d, J=4.6 Hz, 2H); LCMS: (M+H⁺): 488.2, LCMS purity: 97.61%; HPLC purity: 94.47%.

### Example 84-B. 7-((1R,2S,3R,4S)-2,3-dihydroxy-4-(2-(2-(methylamino)quinolin-7-yl)ethyl)cyclopentyl)-1,7-dihydro-4H-pyrrolo[2,3-d]pyrimidin-4-one O-methyl oxime (84-B)

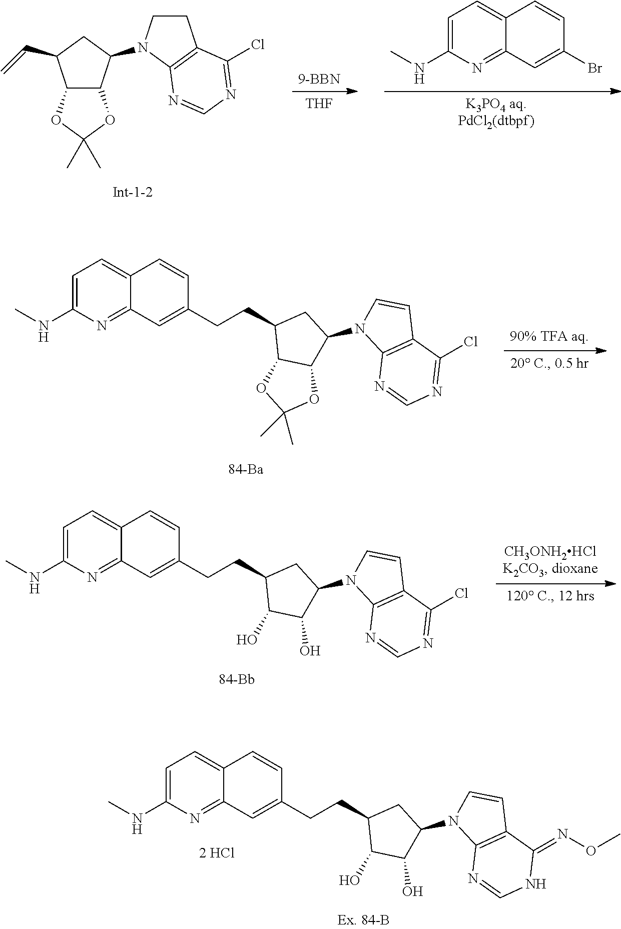

### a) Synthesis of Compound 84-Ba

A solution of Int-1-2 (0.2 g, 625 umol, 1 eq.) and 9-BBN (0.5 M, 3.75 mL, 3 eq.) was stirred at 75° C. for 45 min. LC-MS showed Int-1-2 was consumed completely. One main peak with desired MS was detected. Without work up and purification, the reaction solution was used in the next step directly. To the reaction solution was added K₃PO₄ (402 mg, 1.90 mmol, 3.04 eq.), 7-bromo-N-methyl-quinolin-2-amine (177.42 mg, 748.32 umol, 1.2 eq.), and ditert-butyl (cyclopentyl) phosphane; dichloropalladium; iron (40.64 mg, 62.36 umol, 0.1 eq.), H₂O (0.8 mL) and THE (4 mL). The mixture was stirred at 75° C. for 12 hr. LC-MS showed several new peaks and one main peak with desired MS was detected. The mixture was extracted with EtOAc (30 mL*3) and washed with saturated NaCl (20 mL). The combined organic phase was dried with anhydrous Na₂SO₄ and concentrated in vacuo. The residue was purified by column chromatography (SiO₂, Petroleum ether/Ethyl acetate=50:1 to 1:1). Compound 84-Ba (0.1 g, 171.55 umol, 27.51% yield, 82% purity) was obtained as a yellow oil. ¹H NMR (400 MHz, DMSO-d6) δ=8.63 (s, 1H) 7.93-7.99 (m, 1H) 7.74 (d, J=9.04 Hz, 1H) 7.49 (d, J=7.94 Hz, 1H) 7.35 (s, 1H) 7.01 (d, J=7.94 Hz, 1H) 6.90 (br d, J=4.85 Hz, 1H) 6.71 (d, J=3.53 Hz, 1H) 6.64 (d, J=8.82 Hz, 1H) 5.05 (br d, J=7.06 Hz, 1H) 4.87-4.94 (m, 1H) 3.57 (br t, J=6.50 Hz, 3H) 2.85 (d, J=4.85 Hz, 3H) 2.72 (br t, J=7.61 Hz, 2H) 2.64 (br d, J=1.76 Hz, 1H) 1.72-1.76 (m, 3H) 0.81 (dt, J=16.87, 6.89 Hz, 3H); LCMS: (M+H⁺): 478.0.

### b) Synthesis of compound 84-Bb

A solution of compound 84-Ba (0.1 g, 209.21 umol, 1 eq.) in TFA (0.45 mL) and H₂O (0.05 mL) was stirred at 20° C. for 0.5 hr. LC-MS showed compound 84-Ba was consumed completely. One main peak with desired MS was detected. The reaction was concentrated in vacuo. The residue was dissolved in EtOAc (5 mL), and saturated NaHCO₃ (10 mL) was added dropwise. The mixture was washed with saturated NaCl solution (10 mL*2), extracted with EtOAc (10 mL*3). The combined organic phase was dried with anhydrous Na₂SO₄, concentrated in vacuo. No purification, alkaline compound 84-Bb (0.1 g, crude) was obtained as a yellow solid. LCMS: (M+H⁺): 438.0.

### c) Synthesis of 7-((1R,2S,3R,4S)-2,3-dihydroxy-4-(2-(2-(methylamino)quinolin-7-yl)ethyl)cyclopentyl)-1,7-dihydro-4H-pyrrolo[2,3-d]pyrimidin-4-one O-methyl oxime (84-B)

To a solution of compound 84-Bb (0.07 g, 165.14 umol, 1 eq.) in dioxane (53 mL) was added O-methyl hydroxylamine; hydrochloride (1.38 g, 16.51 mmol, 1.25 mL, 100 eq.) and K₂CO₃ (3.42 g, 24.77 mmol, 150 eq.). The mixture was stirred at 120° C. for 12 hr. LC-MS showed compound 84-Bb was consumed completely. Several new peaks were shown on LC-MS desired compound was detected. The reaction was concentrated in vacuo. The residue was dissolved in MeOH (20 mL), filtered and concentrated in vacuo. The residue was purified by prep-HPLC. Ex. 84-B (0.0249 g, 43.93 umol, 26.60% yield, 92% purity, 2 HCl salt) was obtained as a brown solid. ¹H NMR (400 MHz, DMSO-d6) δ=12.86 (br s, 1H) 9.89 (br s, 1H) 8.09-8.30 (m, 2H) 8.01 (br s, 1H) 7.82 (br d, J=8.19 Hz, 1H) 7.64 (br s, 1H) 7.39 (br d, J=7.70 Hz, 1H) 6.97-7.17 (m, 1H) 6.80 (br s, 1H) 4.80-5.01 (m, 1H) 4.17-4.28 (m, 1H) 3.86 (s, 3H) 3.73-3.79 (m, 2H) 3.16 (br d, J=3.91 Hz, 2H) 2.71-2.89 (m, 3H) 2.68 (br s, 1H) 2.22-2.36 (m, 1H) 1.84-2.05 (m, 2H) 1.67-1.81 (m, 1H) 1.46-1.63 (m, 1H); ¹H NMR (400 MHz, DMSO-d6) δ=8.14-8.31 (m, 2H) 7.92 (br s, 1H) 7.82 (br d, J=7.83 Hz, 1H) 7.64 (br d, J=3.30 Hz, 1H) 7.39 (br d, J=7.95 Hz, 1H) 7.05 (br d, J=9.05 Hz, 1H) 6.76 (d, J=3.42 Hz, 1H) 4.80-5.00 (m, 1H) 4.20 (dd, J=8.01, 5.93 Hz, 1H) 3.86 (s, 3H) 3.73-3.78 (m, 1H) 3.13 (br s, 3H) 2.74-2.89 (m, 3H) 2.24-2.36 (m, 1H) 1.84-2.03 (m, 2H) 1.69-1.82 (m, 1H) 1.47-1.61 (m, 1H); LCMS: (M+H⁺): 449.2; HPLC purity: 88.74%.

### Example 85-B. 7-((1R,2S,3R,4S)-4-(2-(2-aminoquinolin-7-yl)ethyl)-2,3-dihydroxycyclopentyl)-1,7-dihydro-4H-pyrrolo[2,3-d]pyrimidin-4-one O-methyl oxime (85-B)

Ex. 85-B (2HCl salt), a brown solid, was prepared similarly to that of Ex. 84-B. ¹H NMR (400 MHz, DMSO-d6) δ=14.23 (br s, 1H) 8.78-9.33 (m, 1H) 8.34 (d, J=9.29 Hz, 1H) 8.28 (s, 1H) 7.85 (d, J=8.19 Hz, 1H) 7.67 (d, J=3.55 Hz, 1H) 7.55 (s, 1H) 7.40 (dd, J=8.13, 1.16 Hz, 1H) 7.06 (d, J=9.29 Hz, 1H) 6.83 (d, J=3.42 Hz, 1H) 4.81-5.01 (m, 1H) 4.15-4.28 (m, 1H) 3.87 (s, 3H) 3.70-3.80 (m, 1H) 2.75-2.90 (m, 2H) 2.65-2.70 (m, 1H) 2.23-2.36 (m, 1H) 1.83-2.01 (m, 3H) 1.70-1.81 (m, 1H) 1.68-1.68 (m, 1H) 1.51-1.61 (m, 1H); ¹H NMR (400 MHz, DMSO-d6) δ=8.30 (br d, J=9.26 Hz, 1H) 8.23 (s, 1H) 7.80 (br d, J=7.94 Hz, 1H) 7.62 (br d, J=3.53 Hz, 1H) 7.48 (s, 1H) 7.35 (br d, J=8.16 Hz, 1H) 6.98 (d, J=9.26 Hz, 1H) 6.72 (d, J=3.31 Hz, 1H) 4.74-5.01 (m, 1H) 4.15 (br dd, J=8.16, 5.95 Hz, 1H) 3.81 (s, 3H) 2.69-2.85 (m, 2H) 2.14-2.35 (m, 1H) 1.77-2.00 (m, 2H) 1.71 (br d, J=5.51 Hz, 1H) 1.41-1.60 (m, 1H); LCMS: (M+H⁺): 435.2; LCMS purity: 97.88%; HPLC purity: 100.00%.

### Example 86-B. (Z)-7-((1R,2S,3R,4S)-4-(2-(2-amino-3-chloroquinolin-7-yl)ethyl)-2,3-dihydroxycyclopentyl)-3,7-dihydro-4H-pyrrolo[2,3-d]pyrimidin-4-one O-methyl oxime (86-B)

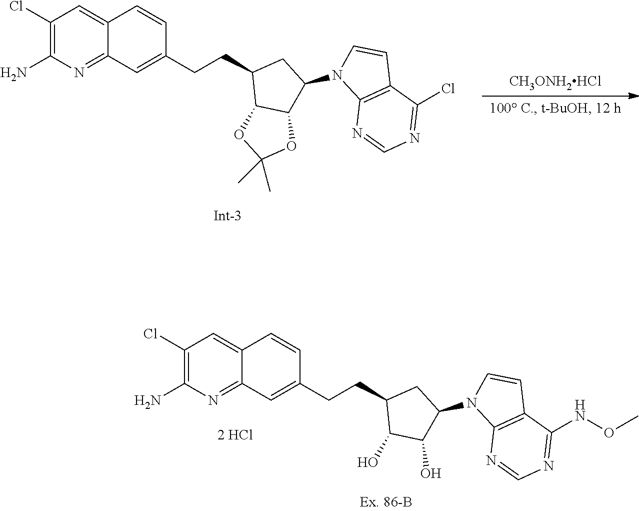 [SMILES: CC1(C)O[C@@H]2[C@@H](CCc3ccc4cc(Cl)c(N)nc4c3)C[C@@H](n3ccc4c(Cl)ncnc43)[C@@H]2O1 ; "CH3ONH2·HCl" ; "100° C., t-BuOH, 12 h" ; CONc1ncnc2c1ccn2[C@@H]1C[C@H](CCc2ccc3cc(Cl)c(N)nc3c2)[C@@H](O)[C@H]1O]

To a solution of Int-3 (0.04 g, 80.26 umol, 1 eq.) in t-BuOH (2 mL) was added O-methyl hydroxyl amine hydrochloride (53.62 mg, 642 umol, 48.75 uL, 8 eq.). The mixture was stirred at 100° C. for 12 h. LC-MS showed compound 15 was consumed completely. Several new peaks were shown on LC-MS and desired compound was detected. The mixture was concentrated in vacuo, and dissolved in THE (1 mL). The mixture was purified by prep-HPLC (HCl condition, column: Luna C18 100*30 5 u; mobile phase: [water (0.05% HCl)−ACN]; B %: 10%-40%, 10 min). Ex. 86-B (0.01786 g, 36.87 umol, 45.94% yield, 96.8% HPLC purity, 2HCl salt) was obtained as a brown solid. ¹H NMR (400 MHz, DMSO-d₆) δ=8.72 (br s, 1H), 8.69 (s, 1H), 8.24 (s, 1H), 7.81 (d, J=8.33 Hz, 1H), 7.64 (br d, J=3.51 Hz, 1H), 7.54 (s, 1H), 7.40 (d, J=8.33 Hz, 1H), 6.79 (d, J=3.07 Hz, 1H), 4.81-4.95 (m, 1H), 4.11-4.22 (m, 1H), 3.84 (s, 2H), 3.68-3.76 (m, 1H), 2.75-2.84 (m, 2H), 2.18-2.29 (m, 1H), 1.89 (br d, J=6.58 Hz, 2H), 1.66-1.76 (m, 1H), 1.46-1.59 (m, 1H); ¹H NMR (400 MHz, DMSO-d₆+D₂O) δ=8.67 (s, 1H), 8.24 (s, 1H), 7.81 (d, J=8.33 Hz, 1H), 7.63 (d, J=3.51 Hz, 1H), 7.54 (s, 1H), 7.40 (d, J=8.77 Hz, 1H), 6.72 (d, J=3.07 Hz, 1H), 4.78-4.95 (m, 1H), 4.13-4.20 (m, 1H), 3.83 (s, 3H), 3.69-3.75 (m, 1H), 2.73-2.84 (m, 2H), 2.16-2.28 (m, 1H), 1.88 (br s, 2H), 1.72 (br d, J=8.77 Hz, 1H), 1.44-1.57 (m, 1H); LCMS: (M+H⁺): 469.2; HPLC purity: 96.80%.

### Example 87-B. 7-((1R,2S,3R,4S)-4-(2-(2-((cyclopropylmethyl)amino)quinolin-7-yl)ethyl)-2,3-dihydroxycyclopentyl)-1,7-dihydro-4H-pyrrolo[2,3-d]pyrimidin-4-one oxime (87-B)

Ex. 87-B was prepared similarly to that of Ex. 79-B. ¹H NMR (400 MHz, DMSO-d₆) δ=7.80 (s, 1H), 7.53 (d, J=8.8 Hz, 1H), 7.27 (d, J=8.2 Hz, 1H), 7.09 (s, 1H), 7.03 (d, J=3.5 Hz, 1H), 6.84-6.73 (m, 2H), 6.67 (br s, 2H), 6.48 (d, J=9.0 Hz, 1H), 6.31 (d, J=3.5 Hz, 1H), 4.64-4.48 (m, 2H), 4.41 (br d, J=4.6 Hz, 1H), 4.02-3.91 (m, 1H), 3.52 (br d, J=4.6 Hz, 1H), 3.02 (t, J=6.1 Hz, 2H), 2.57-2.38 (m, 2H), 2.02 (td, J=7.8, 12.6 Hz, 1H), 1.80-1.57 (m, 2H), 1.54-1.40 (m, 1H), 1.36-1.21 (m, 1H), 0.94-0.77 (m, 1H), 0.28-0.17 (m, 2H), 0.07-0.05 (m, 2H); ¹H NMR (400 MHz, DMSO-d₆+D₂O) δ=7.80 (s, 1H), 7.55 (d, J=8.8 Hz, 1H), 7.29 (d, J=7.9 Hz, 1H), 7.11 (s, 1H), 7.04 (d, J=3.5 Hz, 1H), 6.80 (dd, J=1.2, 8.0 Hz, 1H), 6.49 (d, J=9.0 Hz, 1H), 6.34 (d, J=3.5 Hz, 1H), 4.65-4.52 (m, 1H), 4.03-3.92 (m, 1H), 3.52 (br t, J=5.1 Hz, 1H), 3.02 (d, J=6.8 Hz, 2H), 2.58-2.40 (m, 2H), 2.02 (td, J=7.7, 12.5 Hz, 1H), 1.80-1.58 (m, 2H), 1.56-1.41 (m, 1H), 1.35-1.22 (m, 1H), 0.94-0.81 (m, 1H), 0.29-0.19 (m, 2H), 0.06-0.03 (m, 2H); LCMS purity: 94.46%; HPLC purity: 94.92%.

### Example 88-B. 7-((1R,2S,3R,4R)-4-((S)-(3,4-difluorophenyl)(hydroxy)methyl)-2,3-dihydroxycyclopentyl)-1,7-dihydro-4H-pyrrolo[2,3-d]pyrimidin-4-one oxime (88-B)

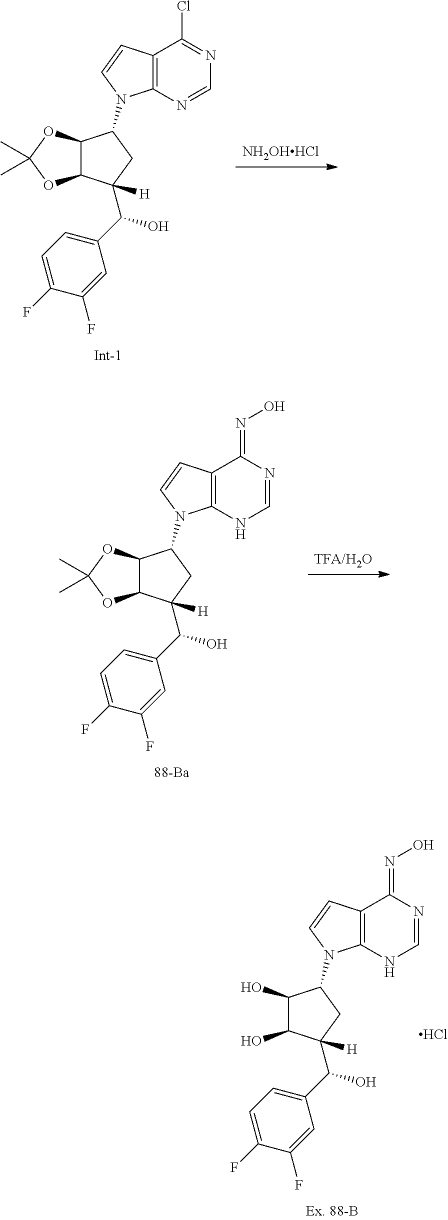 [SMILES: CC1(C)O[C@@H]2[C@@H]([C@H](O)c3ccc(F)c(F)c3)C[C@@H](n3ccc4c(Cl)ncnc43)[C@@H]2O1 ; "NH2OH·HCl" ; CC1(C)O[C@@H]2[C@@H]([C@H](O)c3ccc(F)c(F)c3)C[C@@H](n3ccc4/c(=N/O)nc[nH]c43)[C@@H]2O1 ; "TFA/H2O" ; O/N=c1\nc[nH]c2c1ccn2[C@@H]1C[C@H]([C@H](O)c2ccc(F)c(F)c2)[C@@H](O)[C@H]1O ; Cl]

### a) Synthesis of 7-[(3aS,4R,6R,6aR)-6-[(S)-(3,4-difluorophenyl)-hydroxy-methyl]-2,2-dimethyl-4,5,6,6a-tetrahydro-3aH-cyclopenta[d][1,3]dioxol-4-yl]-1H-pyrrolo[2,3-d]pyrimidin-4-one oxime (88-Ba)

To a solution of (S)-[(3aS,4R,6R,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]-(3,4-difluorophenyl)methanol (Int-1) (80.0 mg, 0.18 mmol) in ethanol (5.0 mL) was added TEA (370.8 mg, 3.67 mmol) and hydroxylamine hydrochloride (127.6 mg, 1.84 mmol). The mixture stirred at 80° C. for 16 hrs. LC-MS showed about 65% product was in the reaction mixture. The solvent was removed under vacuum, diluted by H₂O (10.0 mL) and DCM (30.0 mL), separated, the DCM layer was washed with H₂O (10.0 mL×2) and NaCl aqueous solution (saturated, 20.00 mL), dried over Na₂SO₄, filtered, and concentrated under vacuum to afford 7-[(3aS,4R,6R,6aR)-6-[(S)-(3,4-difluorophenyl)-hydroxy-methyl]-2,2-dimethyl-4,5,6,6a-tetrahydro-3aH-cyclopenta[d][1,3]dioxol-4-yl]-1H-pyrrolo[2,3-d]pyrimidin-4-one oxime (88-Ba) (80.00 mg, crude). LCMS [M+H]: 433.3.

### b) Synthesis of 7-((1R,2S,3R,4R)-4-((S)-(3,4-difluorophenyl)(hydroxy)methyl)-2,3-dihydroxycyclopentyl)-1,7-dihydro-4H-pyrrolo[2,3-d]pyrimidin-4-one oxime (88-B)

To a solution of 7-[(3aS,4R,6R,6aR)-6-[(S)-(3,4-difluorophenyl)-hydroxy-methyl]-2,2-dimethyl-4,5,6,6a-tetrahydro-3aH-cyclopenta[d][1,3]dioxol-4-yl]-1H-pyrrolo[2,3-d]pyrimidin-4-one oxime (88-Ba) (80.0 mg, 0.19 mmol) in water (2.00 mL) was added TFA (1.30 mL, 16.87 mmol). The mixture was stirred at 25° C. for 0.5 h. LC-MS showed the reaction was complete. The mixture was purified by prep-HPLC (0.1% TFA, H₂O:CH₃CN from 90:10 to 5:95) to afford 7-[(1R,2S,3R,4R)-4-[(S)-(3,4-difluorophenyl)-hydroxy-methyl]-2,3-dihydroxy-cyclopentyl]-1H-pyrrolo[2,3-d]pyrimidin-4-one oxime hydrochloride (Ex. 88-B) (12 mg, 0.03 mmol, 14.39% yield) as light yellow solid. LCMS [M+H]: 393.3. ¹HNMR (DMSO-d6+D₂O, 400 MHz): δ 8.26 (s, 1H), 7.63-7.64 (m, 1H), 7.32-7.42 (m, 2H), 7.23-7.24 (m, 1H), 6.85-6.86 (m, 1H), 4.91-4.99 (m, 1H), 4.55-4.57 (m, 1H), 4.19-4.23 (m, 1H), 3.91-3.93 (m, 1H), 2.22-2.28 (m, 1H), 1.96-2.03 (m, 1H), 1.53-1.61 (m, 1H).

### Example 89-B. 7-((1R,2S,3R,4R)-4-((S)-(3,4-difluorophenyl)(hydroxy)methyl)-2,3-dihydroxycyclopentyl)-1,7-dihydro-4H-pyrrolo[2,3-d]pyrimidin-4-one O-methyl oxime (89-B)

To a solution of (S)-[(3aS,4R,6R,6aR)-4-(4-chloropyrrolo[2,3-d]pyrimidin-7-yl)-2,2-dimethyl-4, 5,6,6a-tetrahydro-3aH-cyclopenta[d][1,3]dioxol-6-yl]-(3,4-difluorophenyl)methanol (Int-1) (80.00 mg, 0.18 mmol) in 1-butanol (2.00 mL) was added O-methylhydroxylamine hydrochloride (80.00 mg, 0.96 mmol) and K₂CO₃ (200.00 mg, 1.45 mmol). The mixture was stirred at 100° C. for 2 hrs. LC-MS (ZYX001-86-R1) showed 30% of product was in the reaction mixture. The mixture was purified by prep-HPLC (0.1% TFA, H₂O:CH₃CN from 90:10 to 5:95), then 0.05 mL of conc. HCl was added and lyophilized to afford (1S,2R,3R,5R)-3-[(S)-(3,4-difluorophenyl)-hydroxy-methyl]-5-[(4Z)-4-methoxyimino-1H-pyrrolo[2,3-d]pyrimidin-7-yl]cyclopentane-1,2-diol hydrochloride (Ex. 89-B) (4.20 mg, 0.009 mmol, 5.01% yield) as yellow solid. LCMS [M+H]: 407.2. ¹HNMR (DMSO-d6+D₂O, 400 MHz): δ 8.25 (s, 1H), 7.59-7.60 (m, 1H), 7.34-7.43 (m, 2H), 7.23-7.24 (m, 1H), 6.71-6.73 (m, 1H), 4.91-4.98 (m, 1H), 4.56-4.58 (m, 1H), 4.20-4.24 (m, 1H), 3.89-3.95 (m, 1H), 3.86 (s, 3H), 2.22-2.28 (m, 1H), 1.96-2.04 (m, 1H), 1.52-1.60 (m, 1H).

### Example 90-B. 7-((1R,2S,3R,4S)-4-(2-(2-((cyclopropylmethyl)amino)quinolin-7-yl)ethyl)-2,3-dihydroxycyclopentyl)-1,7-dihydro-4H-pyrrolo[2,3-d]pyrimidin-4-one O-methyl oxime (90-B)

Example 90-B (HCl salt) was prepared similarly to that of 87-B. ¹H NMR (400 MHz, DMSO-d₆) δ=12.98 (br s, 1H), 9.87 (br s, 1H), 8.32-8.16 (m, 2H), 8.04 (br s, 1H), 7.81 (br d, J=7.7 Hz, 1H), 7.66 (br d, J=2.3 Hz, 1H), 7.45-7.34 (m, 1H), 7.22-7.08 (m, 1H), 6.84 (br s, 1H), 5.00-4.85 (m, 1H), 4.21 (dd, J=5.9, 8.1 Hz, 1H), 3.87 (s, 2H), 3.78-3.73 (m, 2H), 2.90-2.71 (m, 2H), 2.35-2.23 (m, 1H), 2.35-2.23 (m, 1H), 2.03-1.83 (m, 2H), 1.81-1.67 (m, 1H), 1.62-1.48 (m, 1H), 1.25-1.08 (m, 1H), 0.59 (br d, J=7.3 Hz, 2H), 0.37 (q, J=4.6 Hz, 2H); ¹H NMR (400 MHz, DMSO-d₆+D₂O) δ=8.31-8.15 (m, 2H), 7.90 (br s, 1H), 7.79 (br d, J=8.3 Hz, 1H), 7.65 (d, J=3.5 Hz, 1H), 7.36 (br d, J=8.3 Hz, 1H), 7.08 (br d, J=9.2 Hz, 1H), 6.77 (d, J=3.5 Hz, 1H), 4.95-4.83 (m, 1H), 4.18 (dd, J=5.7, 8.3 Hz, 1H), 3.84 (s, 3H), 3.76-3.70 (m, 1H), 2.78 (dt, J=7.0, 14.0 Hz, 2H), 2.34-2.19 (m, 1H), 2.00-1.80 (m, 2H), 1.74 (br d, J=3.5 Hz, 1H), 1.59-1.45 (m, 1H), 1.16 (br s, 1H), 0.57 (br d, J=7.0 Hz, 2H), 0.39-0.27 (m, 2H); LCMS: (M+H⁺): 489.2; LCMS purity: 96.0%;

### Example 91-B. (1S,2R,3S,5R)-3-(2-(2-((cyclopropylmethyl)amino)quinolin-7-yl)ethyl)-5-((Z)-4-(2-methylhydrazineylidene)-1,4-dihydro-7H-pyrrolo[2,3-d]pyrimidin-7-yl)cyclopentane-1,2-diol (91-B)

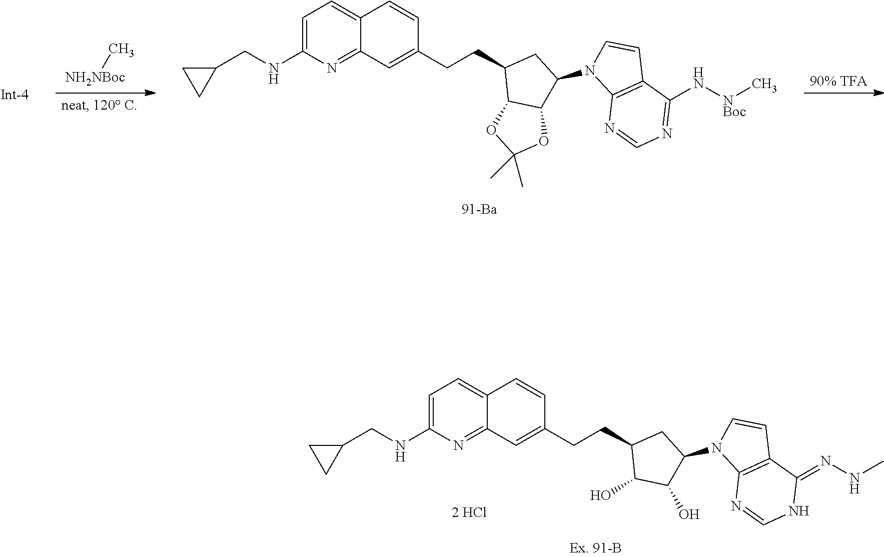

### a) Synthesis of Compound 91-Ba

A mixture of Int-4 (0.06 g, 115.82 umol, 1 eq), N-methyl-N-Boc hydrazine (2.96 g, 20.21 mmol, 3 mL, 174.53 eq) was purged with N₂ for 3 times, and then the mixture was stirred at 110° C. for 12 hr under N₂ atmosphere. LCMS showed it was almost completed and the desired product was detected. The mixture was lyophilized to give a brown oil (200 mg), which was purified with by prep-HPLC under alkaline condition to give a white solid (45 mg).

### b) Synthesis of (1S,2R,3S,5R)-3-(2-(2-((cyclopropylmethyl)amino)quinolin-7-yl)ethyl)-5-((Z)-4-(2-methylhydrazineylidene)-1,4-dihydro-7H-pyrrolo[2,3-d]pyrimidin-7-yl)cyclopentane-1,2-diol (91-B)

A mixture of compound 91-Ba (30 mg, 47.79 umol, 1 eq) in TFA (0.9 mL) and H₂O (0.1 mL) was degassed and purged with N₂ for 3 times, and then the mixture was stirred at 20° C. for 5 min under N₂ atmosphere. LCMS showed it was completed. It was concentrated at 25° C. and purified by prep-HPLC under acid condition to give a white solid (column: Boston Green ODS 150*30 5 u; mobile phase: [water(0.05% HCl)-ACN]; B %: 10%-40%, 11 min). 19.37 mg Ex. 91-B was obtained as a white solid. ¹H NMR (400 MHz, DMSO-d6) δ ppm 12.93 (br s, 1H) 11.27 (br s, 1H) 9.82 (br s, 1H) 8.16-8.28 (m, 2H) 8.01 (br s, 1H) 7.81 (br d, J=7.95 Hz, 1H) 7.70 (d, J=3.55 Hz, 1H) 7.38 (br d, J=8.31 Hz, 1H) 7.12 (br d, J=9.05 Hz, 1H) 6.98 (br d, J=3.30 Hz, 1H) 4.80-5.04 (m, 1H) 4.21 (dd, J=8.19, 5.87 Hz, 1H) 3.71-3.83 (m, 1H) 2.74-2.92 (m, 2H) 2.67 (s, 4H) 2.21-2.36 (m, 1H) 1.70-2.04 (m, 3H) 1.48-1.63 (m, 1H) 1.11-1.25 (m, 1H) 0.59 (br d, J=7.09 Hz, 2H) 0.37 (br d, J=4.89 Hz, 2H); ¹H NMR (400 MHz, DMSO-d6) δ ppm 8.17-8.31 (m, 2H) 7.89 (br s, 1H) 7.81 (br d, J=8.07 Hz, 1H) 7.69 (d, J=3.67 Hz, 1H) 7.38 (br d, J=7.95 Hz, 1H) 7.09 (br d, J=9.54 Hz, 1H) 6.93 (br d, J=3.18 Hz, 1H) 4.80-5.01 (m, 1H) 4.20 (dd, J=8.25, 5.93 Hz, 1H) 3.70-3.79 (m, 1H) 2.72-2.91 (m, 2H) 2.61-2.69 (m, 3H) 2.20-2.37 (m, 1H) 1.68-2.02 (m, 3H) 1.47-1.62 (m, 1H) 1.20 (br d, J=18.71 Hz, 1H) 0.59 (br d, J=6.97 Hz, 2H) 0.36 (q, J=4.69 Hz, 2H); LCMS: (M+H⁺): 488.2; LCMS purity: 97.0%; HPLC purity: 93.9%.

### Example 92-B. 7-((1R,2S,3R,4R)-4-((S)-(3,4-dichlorophenyl)(hydroxy)methyl)-2,3-dihydroxycyclopentyl)-1,7-dihydro-4H-pyrrolo[2,3-d]pyrimidin-4-one O-methyl oxime (92-B)

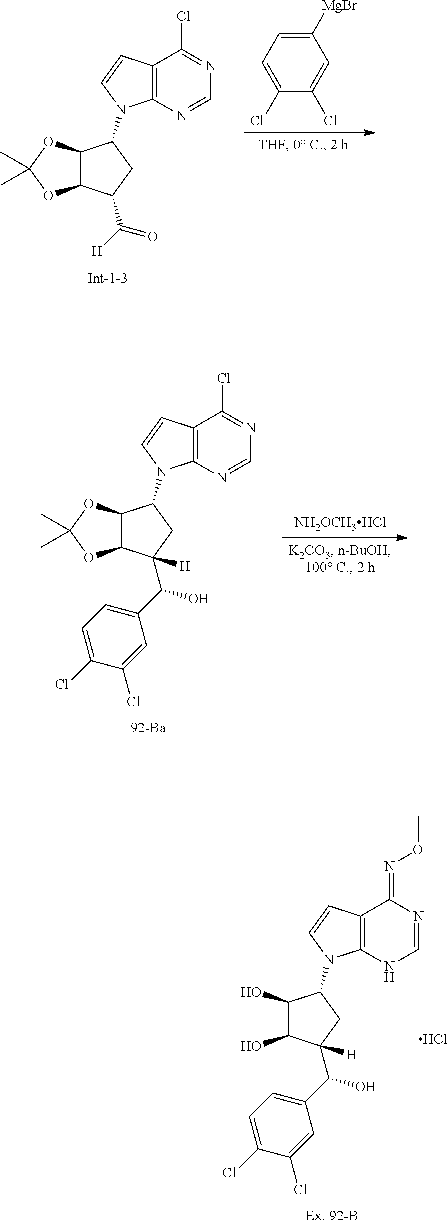

### a) Synthesis of (S)-[(3aS,4R,6R,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]-(3,4-dichlorophenyl)methanol (92-Ba)

To a solution of (3aS,4R,6S,6aR)-4-(4-chloropyrrolo[2,3-d]pyrimidin-7-yl)-2,2-dimethyl-4,5,6,6a-tetrahydro-3aH-cyclopenta[d][1,3]dioxole-6-carbaldehyde (Int-1-3) (2.63 g, 3.76 mmol) in THF (20.0 mL) was added bromo-(3,4-dichlorophenyl)magnesium (22.5 mL, 11.27 mmol) at 0° C. The reaction mixture was stirred at 0° C. for 1 h. LCMS showed the reaction was completed. The reaction mixture was added H₂O (30.0 mL) and EA (60.0 mL), washed with H₂O (30.0 mL), brine (30.0 mL), dried over Na₂SO₄, filtered, concentrated in vacuum to give crude product which was purified by silica gel column chromatography (PE:EA=5:1 to 3:1) to give (S)-[(3aS,4R,6R,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]-(3,4-dichlorophenyl)methanol (92-Ba) (450.0 mg, 0.96 mmol, 25.6% yield). LCMS [M+H]: 468.1.

### b) Synthesis of (1S,2R,3R,5R)-3-[(S)-(3,4-dichlorophenyl)-hydroxy-methyl]-5-[(4Z)-4-methoxyimino-1H-pyrrolo[2,3-d]pyrimidin-7-yl]cyclopentane-1,2-diol hydrochloride (Ex. 94-B)

To a solution of (S)-[(3aS,4R,6R,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]-(3,4-dichlorophenyl)methanol (150.0 mg, 0.32 mmol) in 1-butanol (5.0 mL) was added O-Methylhydroxylamine hydrochloride (133.6 mg, 1.60 mmol), K₂CO₃ (353.3 mg, 2.56 mmol). The mixture was stirred at 100° C. for 2 h. LCMS showed the reaction was completed. The mixture was purified by prep-HPLC to afford (1S,2R,3R,5R)-3-[(S)-(3,4-dichlorophenyl)-hydroxy-methyl]-5-[(4Z)-4-methoxyimino-1H-pyrrolo[2,3-d]pyrimidin-7-yl]cyclopentane-1,2-diol hydrochloride (Ex. 94-B) (16.0 mg, 0.03 mmol, 10.1% yield) as a white solid. LCMS [M+H]: 439.1. ¹H NMR (400 MHz, DMSO-d₆+D₂O): δ 8.59 (s, 1H), 7.58-7.63 (m, 3H), 7.38-7.40 (m, 1H), 6.72-6.73 (m, 1H), 4.91-4.98 (m, 1H), 4.58-4.60 (m, 1H), 4.19-4.23 (m, 1H), 3.89-3.90 (m, 1H), 3.86 (s, 3H), 2.22-2.28 (m, 1H), 1.99-2.07 (m, 1H), 1.55-1.63 (m, 1H).

### Example 93-B. 7-((1R,2S,3R,4R)-4-((S)-(3,4-dichlorophenyl)(hydroxy)methyl)-2,3-dihydroxycyclopentyl)-1,7-dihydro-4H-pyrrolo[2,3-d]pyrimidin-4-one oxime (93-B)

Example 93-B, a light yellow solid, was prepared similarly to that of Ex. 92-B. LCMS [M+H]: 425.1. ¹H NMR (400 MHz, DMSO-d₆+D₂O): δ 8.27 (s, 1H), 7.58-7.64 (m, 3H), 7.38-7.40 (m, 1H), 6.80-6.81 (m, 1H), 4.93-5.00 (m, 1H), 4.58-4.60 (m, 1H), 4.19-4.23 (m, 1H), 3.90-3.91 (m, 1H), 2.23-2.28 (m, 1H), 2.00-2.07 (m, 1H), 1.55-1.63 (m, 1H).

### Example 94-B. 7-((1R,2S,3R,4S)-4-((S)-1-(3,4-dichlorophenyl)-1-hydroxyethyl)-2,3-dihydroxycyclopentyl)-1,7-dihydro-4H-pyrrolo[2,3-d]pyrimidin-4-one oxime (94-B)

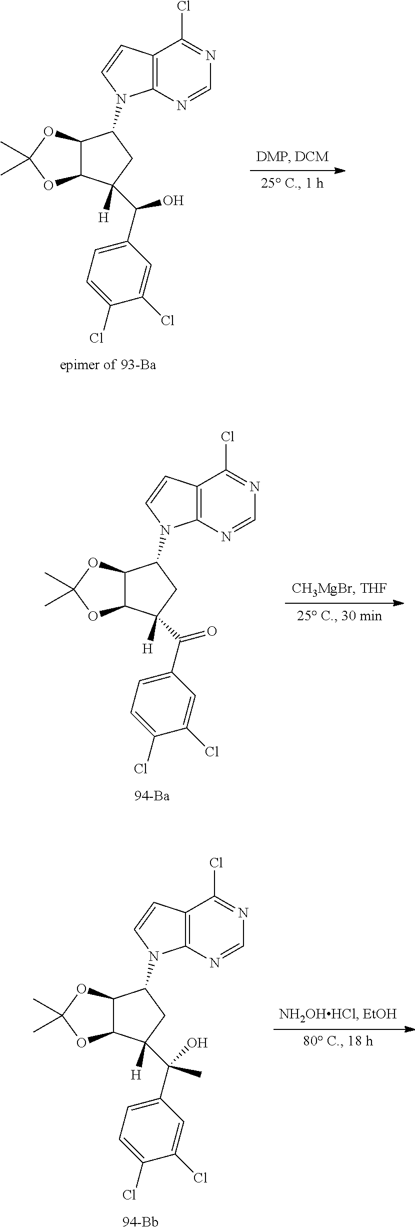

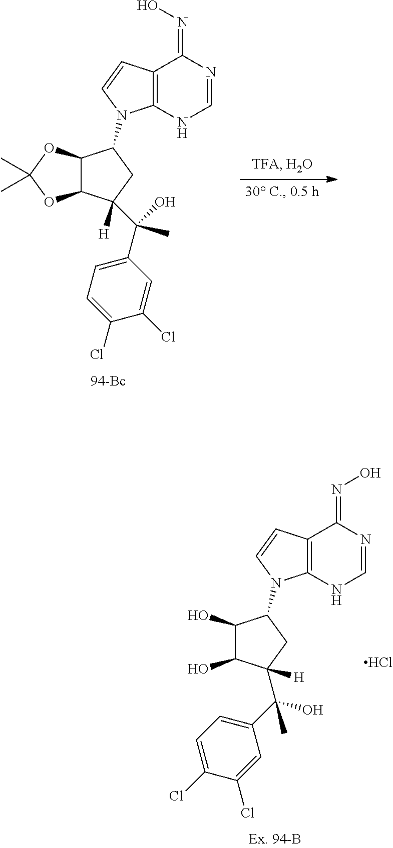

### a) Synthesis of [(3aS,4R,6S,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]-(3,4-dichlorophenyl)methanone (94-Ba)

To a solution of (R)-[(3aS,4R,6R,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]-(3,4-dichlorophenyl)methanol (epimer of 93-Ba, isolated from the reaction of step a, Ex. 93-B) (450.0 mg, 0.96 mmol) in DCM (10.0 mL) was added Dess-Martin periodinane (1221.5 mg, 2.88 mmol) at 0° C., then the mixture warmed to 25° C. naturally and stirred at 25° C. for 1 h. LCMS showed the reaction was completed. NaHCO₃aqueous (30.00 mL) was added to the mixture. The reaction mixture was extracted with DCM (50.0 mL×3). The organic layers were dried over Na₂SO₄, filtered, concentrated in vacuum to give crude product which was purified by column (PE:EA=8:1) to give [(3aS,4R,6S,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]-(3,4-dichlorophenyl)methanone (94-Ba) (340.0 mg, 0.73 mmol, 75.9% yield) as a white solid. LCMS [M+H]: 466.1.

### b) Synthesis of (1S)-1-[(3aS,4R,6S,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]-1-(3,4-dichlorophenyl)ethanol (94-Bb)

To a solution of [(3aS,4R,6S,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]-(3,4-dichlorophenyl)methanone (94-Ba) (340.0 mg, 0.73 mmol) in THE (7.00 mL) was dropwise added bromo(methyl)magnesium (0.7 mL, 2.19 mmol) at 0° C., then the mixture warmed to 25° C. naturally and stirred at 25° C. for 30 mins. TLC showed the reaction was completed. The reaction mixture was added NH₄Cl aqueous (10.00 mL), extracted with EA (30.0 mL×3). The organic layers were dried over Na₂SO₄, filtered, concentrated in vacuum to give crude product which was purified by column (PE:EA=7:1 to PE:EA=5:1) to give (1S)-1-[(3aS,4R,6S,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]-1-(3,4-dichlorophenyl)ethanol (94-Bb) (140.0 mg, 0.29 mmol, 39.8% yield). LCMS [M+H]: 482.1.

### c) Synthesis of 7-[(3aS,4R,6S,6aR)-6-[(1S)-1-(3,4-dichlorophenyl)-1-hydroxy-ethyl]-2,2-dimethyl-4,5,6,6a-tetrahydro-3aH-cyclopenta[d][1,3]dioxol-4-yl]-1H-pyrrolo [2,3-d]pyrimidin-4-one oxime (94-Bc)

To a solution of (1S)-1-[(3aS,4R,6S,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]-1-(3,4-dichlorophenyl)ethanol (94-Bb) (140.0 mg, 0.29 mmol) in ethanol (8.00 mL) was added TEA (586.9 mg, 5.80 mmol) and hydroxylamine hydrochloride (201.5 mg, 2.90 mmol), then the mixture was stirred at 80° C. for 16 h. LCMS showed the reaction mixture was done. The reaction mixture was concentrated in vacuum and added EA (50.0 mL). The organic layer was washed with brine (30.00 mL×3), dried over Na₂SO₄, concentrated in vacuum to give crude product 7-[(3aS,4R,6S,6aR)-6-[(1S)-1-(3,4-dichlorophenyl)-1-hydroxy-ethyl]-2,2-dimethyl-4,5,6,6a-tetrahydro-3aH-cyclopenta[d][1,3]dioxol-4-yl]-1H-pyrrolo [2,3-d] pyrimidin-4-one oxime (94-Bc) (140.0 mg, 0.23 mmol, 81.0% yield). LCMS [M+H]: 479.2.

### d) Synthesis of 7-[(1R,2S,3R,4S)-4-[(1S)-1-(3,4-dichlorophenyl)-1-hydroxy-ethyl]-2,3-dihydroxy-cyclopentyl]-1H-pyrrolo[2,3-d]pyrimidin-4-one oxime hydrochloride (Ex. 94-B)

To a solution of 7-[(3aS,4R,6S,6aR)-6-[(1S)-1-(3,4-dichlorophenyl)-1-hydroxy-ethyl]-2,2-dimethyl-4,5,6,6a-tetrahydro-3aH-cyclopenta[d][1,3]dioxol-4-yl]-1H-pyrrolo[2,3-d]pyrimidin-4-one oxime (94-Bc) (140.0 mg, 0.24 mmol) in Water (3.0 mL) was added TFA (2.0 mL, 25.96 mmol), the reaction was stirred at 30° C. for 0.5 h. LCMS showed the reaction was completed. The mixture was sent to pre-HPLC to give 7-[(1R,2S,3R,4S)-4-[(1S)-1-(3,4-dichlorophenyl)-1-hydroxy-ethyl]-2,3-dihydroxy-cyclopentyl]-1H-pyrrolo[2,3-d]pyrimidin-4-one oxime hydrochloride (Ex. 94-B) (23.0 mg, 0.048 mmol, 20.5% yield) as a white solid. LCMS [M+H]: 439.1. ¹H NMR (400 MHz, DMSO-d₆+D₂O): δ 8.27 (s, 1H), 7.73-7.7.75 (m, 1H), 7.59-7.61 (m, 2H), 7.48-7.50 (m, 1H), 6.81-6.82 (m, 1H), 4.97-4.99 (m, 1H), 4.02-4.05 (m, 1H), 3.50-3.51 (m, 1H), 2.36-2.41 (m, 1H), 2.18-2.24 (m, 1H), 1.93-1.95 (m, 1H), 1.36 (s, 3H).

### Examples of Formula V and Formula VI

### Example 1-C. (2R,3R,4S,5S)-2-(6-amino-9H-purin-9-yl)-5-((R)-1-(3,4-dichlorophenyl)-1-hydroxyethyl)tetrahydrofuran-3,4-diol (1-C)

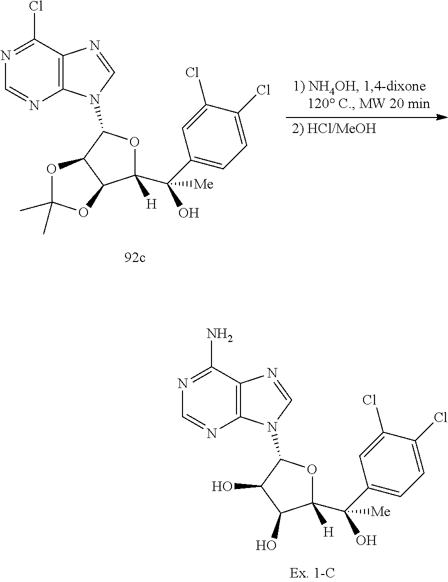

A mixture of (1R)-1-[(3aR,4R,6S,6aR)-4-(6-aminopurin-9-yl)-2,2-dimethyl-3a,4,6,6a-tetrahydrofuro[3,4-d][1,3]dioxol-6-yl]-1-(3,4-dichlorophenyl)ethanol (92c) (160 .mg, 0.3400 mmol) and 1 M HCl in MeOH (1 mL) was stirred at RT overnight. LCMS and TLC (9:1 DCM:MeOH) showed product formed and small amount of remaining st.m. The reaction mixture was concentrated and the crude product was purified on a 12 g column, eluted with 0-14% MeOH/DCM to give (2R,3R,4S,5S)-2-(6-aminopurin-9-yl)-5-[(1R)-1-(3,4-dichlorophenyl)-1-hydroxy-ethyl]tetrahydrofuran-3,4-diol (Ex 1-C) (117 mg, 0.26 mmol, 76% yield) as a light yellow solid. LCMS (M+H+) 426/428/430. ¹HNMR (400 MHz, Methanol-d₄) δ 8.64 (s, 1H), 8.46 (s, 1H), 7.75 (d, J=2.0 Hz, 1H), 7.53-7.45 (m, 2H), 6.12 (d, J=6.8 Hz, 1H), 4.68 (dd, J=6.8, 5.2 Hz, 1H), 4.31 (d, J=2.0 Hz, 1H), 4.03 (dd, J=5.2, 2.0 Hz, 1H), 1.59 (s, 3H).

### Example 2-C. (2R,3R,4S,5S)-2-(6-amino-9H-purin-9-yl)-5-((R)-1-(4-chloro-3-methylphenyl)-1-hydroxyethyl)tetrahydrofuran-3,4-diol (2-C)

Example 2-C was synthesized via similar procedures of Example 1-C except for substituting (3,4-Dichlorophenyl)magnesium bromide with (4-chloro-3-methylphenyl)magnesium bromide in step 2. LCMS (M+H+) 406/408. ¹H NMR (600 MHz, Methanol-d₄) δ 8.51 (s, 1H), 8.46-8.34 (m, 2H), 7.50 (d, J=2.1 Hz, 1H), 7.42-7.22 (m, 2H), 6.07 (d, J=7.3 Hz, 1H), 4.74 (dd, J=5.2, 7.3 Hz, 1H), 4.34 (d, J=1.5 Hz, 1H), 4.00 (dd, J=1.4, 5.2 Hz, 1H), 2.40 (s, 3H), 1.57 (s, 3H).

### Example 3-C. (2R,3R,4S,5S)-2-(4-amino-5-fluoro-7H-pyrrolo[2,3-d]pyrimidin-7-yl)-5-((R)-1-(3,4-dichlorophenyl)-1-hydroxyethyl)tetrahydrofuran-3,4-diol (3-C)

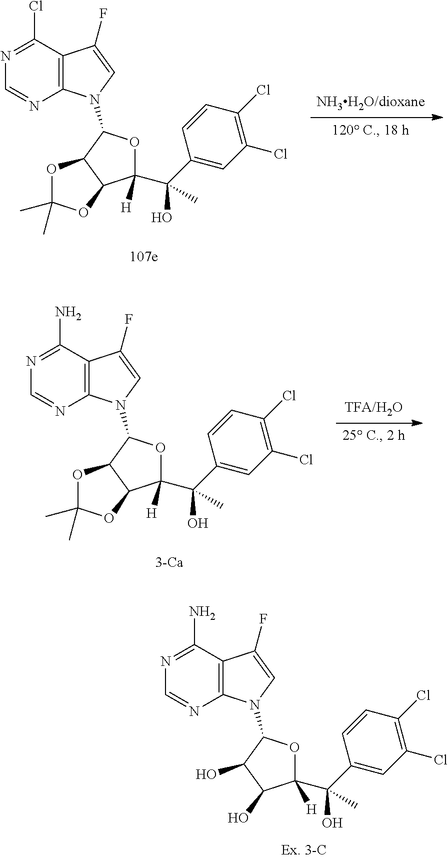

### Step 1. Synthesis of(1R)-1-[(3aR,4R,6S,6aR)-4-(4-amino-5-fluoro-pyrrolo[2,3-d]pyrimidin-7-yl)-2,2-dimethyl-3a,4,6,6a-tetrahydrofuro[3,4-d][1,3]dioxol-6-yl]-1-(3,4-dichlorophenyl) ethanol (3-Ca)

To a solution of (1R)-1-[(3aR,4R,6S,6aR)-4-(4-chloro-5-fluoro-pyrrolo[2,3-d]pyrimidin-7-yl)-2,2-dimethyl-3a,4,6,6a-tetrahydrofuro[3,4-d][1,3]dioxol-6-yl]-1-(3,4-dichlorophenyl)ethanol (107e) (100.0 mg, 0.20 mmol) in 1,4-Dioxane (1.0 mL) was added NH₄OH (2.0 mL, 30.0%, 52.0 mmol). The mixture was stirred at 120° C. for 16 h in sealed tube. The solvent was removed in vacuum to give crude product which was purified by reversed-phase combi-flash eluted with MeCN in water from 10.0% to 70.0 to give (1R)-1-[(3aR,4R,6S,6aR)-4-(4-amino-5-fluoro-pyrrolo[2,3-d]pyrimidin-7-yl)-2,2-dimethyl-3a,4,6,6a-tetrahydrofuro[3,4-d][1,3]dioxol-6-yl]-1-(3,4-dichlorophenyl) ethanol (3-Ca) (80.0 mg, 0.17 mmol, 83.2% yield) as a gray solid. LCMS [M+H]: 483.1.

### Step 2. Synthesis of(2R,3R,4S,5S)-2-(4-amino-5-fluoro-pyrrolo-[2,3-d]pyrimidin-7-yl)-5-[(1R)-1-(3,4-dichlorophenyl)-1-hydroxy-ethyl]tetrahydrofuran-3,4-diol (3-C)

To a solution of (1R)-1-[(3aR,4R,6S,6aR)-4-(4-amino-5-fluoro-pyrrolo[2,3-d]pyrimidin-7-yl)-2,2-dimethyl-3a,4,6,6a-tetrahydrofuro[3,4-d][1,3]dioxol-6-yl]-1-(3,4-dichlorophenyl)ethanol (80.0 mg, 0.17 mmol) in Water (1.0 mL) was added TFA (0.50 mL, 6.73 mmol). The mixture was stirred at 25° C. for 1 h. The solvent was removed in vacuum to give crude which was purified by pre-HPLC, eluted with MeCN in water from MeCN in water (0.1% NH₃.H₂O) from 10.0% to 80.0% to afford (2R,3R,4S,5S)-2-(4-amino-5-fluoro-pyrrolo-[2,3-d]pyrimidin-7-yl)-5-[(1R)-1-(3,4-dichlorophenyl)-1-hydroxy-ethyl]tetrahydrofuran-3,4-diol (Ex. 3-C) (51.1 mg, 0.11 mmol, 68.6% yield) as a white solid. LCMS [M+H]: 443.1. ¹H NMR (400 M Hz, DMSO-d6+D₂O): δ 8.10 (s, 1H), 7.77 (d, J=1.7 Hz, 1H), 7.62 (d, J=8.4 Hz, 1H), 7.52 (d, J=8.4 Hz, 1H), 7.34 (s, 1H), 5.92 (d, J=7.6 Hz, 1H), 4.45-4.42 (m, 1H), 4.09 (brs, 1H), 3.68 (d, J=5.1 Hz, 1H), 1.43 (s, 3H). ¹⁹F NMR (377 μM Hz, DMSO-d6): −168.17 (s, 1F).

### Example 5-C. (2R,3R,4S,5R)-2-(4-amino-7H-pyrrolo[2,3-d]pyrimidin-7-yl)-5-((S)-1-(4-chlorophenyl)ethyl)tetrahydrofuran-3,4-diol hydrochloride (5-C)

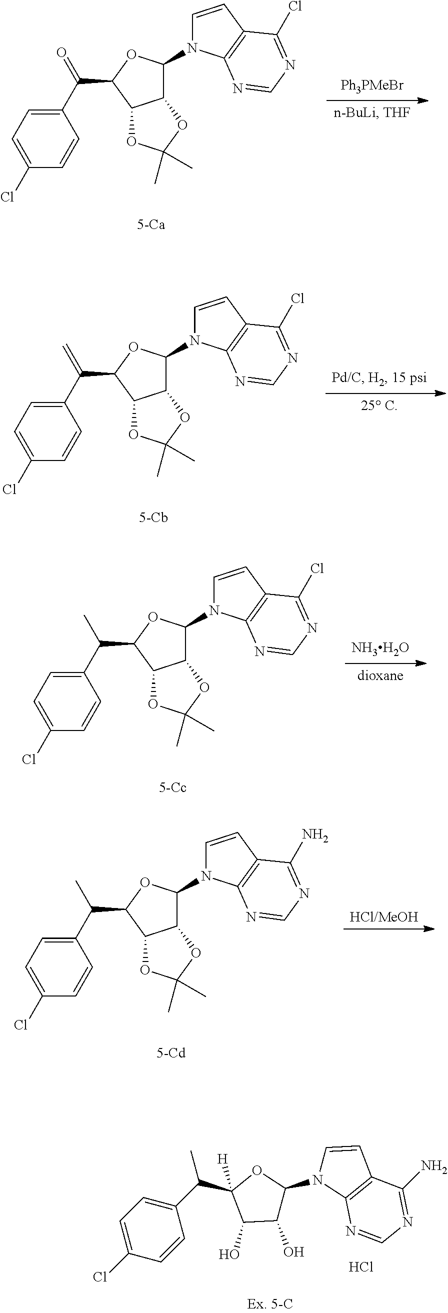

### Step 1. Preparation of 4-chloro-7-((3aR,4R,6R,6aR)-6-(1-(4-chlorophenyl)vinyl)-2,2-dimethyltetrahydrofuro[3,4-d][1,3]dioxol-4-yl)-7H-pyrrolo[2,3-d]pyrimidine (5-Cb)

To a solution of Ph₃PMeBr (428 mg, 1.20 mmol, 1.04 eq.) in THE (15 mL) at −78° C. is added n-BuLi (2.5 M, 479 uL, 1.04 eq.) slowly. The mixture is stirred at 0° C. for 30 minutes, and then cooled to −78° C. To the reaction mixture is added compound 5-Ca (0.5 g, 1.15 mmol, 1 eq.) in THE (4 mL). The reaction mixture is allowed to warm to 25° C. and stirred for 5.5 h. LC-MS showed no compound 5-Ca was remained. Several new peaks were shown on LC-MS and desired compound 5-Cb was detected. The reaction mixture was quenched by addition water (20 mL) at −60° C., and then diluted with EtOAc (20 mL) and extracted with EtOAc (20 mL*2). The combined organic layers were dried over Na₂SO₄, filtered and concentrated under reduced pressure to give a residue. The crude compound 5-Cb (750 mg, crude) was as yellow solid and used into the next step without further purification. LCMS: (M+H⁺): 432.0, 434.0.

### Step 2. Preparation of 4-chloro-7-((3aR,4R,6R,6aR)-6-((S)-1-(4-chlorophenyl)ethyl)-2,2-dimethyltetrahydrofuro[3,4-d][1,3]dioxol-4-yl)-7H-pyrrolo[2,3-d]pyrimidine (5-Cc)

To a solution of compound 5-Cb (50 mg, 116 umol, 1 eq) in THE (2 mL) was added Pd/C (0.01 g, 50% purity) under N₂. The suspension was degassed under vacuum and purged with H₂ several times. The mixture was stirred under H₂ (15 psi) at 25° C. for 15 min. LC-MS showed no compound 5-Cb was remained. Several new peaks were shown on LC-MS and desired compound was detected. The reaction mixture was filtered and the filtrate was concentrated. The crude compound 5-Cc (32 mg, crude) was as yellow oil and used into the next step without further purification. LCMS: (M+H⁺): 434.0, 436.0.

### Step 3. Preparation of 7-((3aR,4R,6R,6aR)-6-((S)-1-(4-chlorophenyl)ethyl)-2,2-dimethyltetrahydrofuro[3,4-d][1,3]dioxol-4-yl)-7H-pyrrolo[2,3-d]pyrimidin-4-amine (5-Cd)

A mixture of compound 5-Cc (32 mg, 74 umol, 1 eq.) in NH₃.H₂O (1.86 g, 13.3 mmol, 2.04 mL, 25% purity, 180 eq.) and dioxane (2 mL) was stirred at 100° C. for 12 h. LC-MS showed no compound 5-Cc was remained. Several new peaks were shown on LC-MS and ˜64% of desired compound was detected. The reaction mixture was concentrated under reduced pressure to remove solvent. The residue was diluted with MeOH (5 mL*5) and concentrated under reduced pressure to give a residue. The crude product compound 5-Cd (30 mg, crude) was as yellow oil and used into the next step without further purification. LCMS: (M+H⁺): 415.1.

### Step 4. Preparation of (2R,3R,4S,5R)-2-(4-amino-7H-pyrrolo[2,3-d]pyrimidin-7-yl)-5-((S)-1-(4-chlorophenyl)ethyl)tetrahydrofuran-3,4-diol hydrochloride (5-C)

To compound 5-Cd (30 mg, 72.31 umol, 1 eq.) was added HCl/MeOH (4 M, 1.81 mL, 100 eq.) in one portion at 0° C. The mixture was stirred at 25° C. for 10 min. LC-MS showed no compound 5-Cd was remained. Several new peaks were shown on LC-MS and desired compound was detected. The reaction mixture was concentrated under reduced pressure to remove solvent. The residue was purified by prep-HPLC (HCl condition). HPLC column: Luna C18 100*30 5 u; mobile phase: [water (0.05% HCl)−ACN]; B %: 20%-40%, 10 min. Compound 5-C (3.54 mg, 8.32 umol, 11.5% yield, 96.63% LCMS purity, HCl) was obtained as a white gum. ¹H NMR (400 MHz, DMSO-d₆) δ=8.34 (s, 1H), 7.42 (d, J=3.1 Hz, 1H), 7.33-7.17 (m, 4H), 6.98 (br d, J=3.1 Hz, 1H), 5.99 (d, J=6.0 Hz, 1H), 4.26 (br t, J=5.4 Hz, 1H), 4.04 (br d, J=4.4 Hz, 1H), 4.01-3.94 (m, 1H), 3.12-3.02 (m, 1H), 1.26 (br d, J=6.8 Hz, 3H); 1H NMR (400 MHz, DMSO-d₆+D₂O) δ=8.32 (s, 1H), 7.45-7.37 (m, 1H), 7.29-7.19 (m, 4H), 6.94 (d, J=3.5 Hz, 1H), 5.98 (d, J=6.0 Hz, 1H), 4.25 (br t, J=5.6 Hz, 1H), 4.07-4.02 (m, 2H), 3.98 (br dd, J=3.7, 8.1 Hz, 3H), 3.10-3.01 (m, 2H), 1.25 (br d, J=7.0 Hz, 3H); LCMS: (M+H⁺): 375.1; HPLC purity: 97.35%; SFC purity: 100.0%.

### Example 15-C. (2R,3R,4S,5S)-2-(4-amino-5-fluoro-7H-pyrrolo[2,3-d]pyrimidin-7-yl)-5-((R)-1-(4-chloro-3-fluorophenyl)-1-hydroxyethyl)tetrahydrofuran-3,4-diol (15-C)

Example 15-C was prepared following the similar procedures of Ex. 3-C except for substituting 107e with (1R)-1-[(3aR,4R,6S,6aR)-4-(4-chloro-5-fluoro-pyrrolo[2,3-d]pyrimidin-7-yl)-2,2-dimethyl-3a,4,6,6a-tetrahydrofuro[3,4-d][1,3]dioxol-6-yl]-1-(4-chloro-3-fluorophenyl)ethanol. LCMS [M+H]: 427.2 ¹H NMR (400 M Hz, DMSO-d6): δ=8.09 (s, 1H), 7.54-7.58 (m, 2H), 7.41 (d, J=8.4 Hz, 1H), 7.37 (s, 1H), 7.12 (brs, 2H), 6.53 (s, 1H), 5.91 (d, J=8.0 Hz, 1H), 5.16 (brs, 1H), 4.84 (brs, 1H), 4.44 (brs, 1H), 4.10 (s, 1H), 3.66 (brs, 1H), 1.42 (s, 3H). ¹H NMR (400 M Hz, DMSO-d6+D₂O): S=8.09 (s, 1H), 7.54-7.58 (m, 2H), 7.41 (d, J=8.4 Hz, 1H), 7.37 (s, 1H), 5.92 (d, J=8.0 Hz, 1H), 4.42-4.46 (m, 1H), 4.09 (s, 1H), 3.66 (d, J=5.2 Hz, 1H), 1.42 (s, 3H). ¹⁹F NMR (377 μM Hz, DMSO-d6): δ −116.62 (s, 1F), −168.22 (s, 1F).

### Example 21-C. (2R,3R,4S,5S)-2-(4-amino-7H-pyrrolo[2,3-d]pyrimidin-7-yl)-5-((S)-1-(5-chlorothiophen-2-yl)-1-hydroxyethyl)tetrahydrofuran-3,4-diol (21-C)

Example 21-C was synthesized via similar procedures of Example 1-C. ¹H NMR (400 MHz, Methanol-d₄) δ 8.09 (s, 1H), 7.23 (d, J=3.7 Hz, 1H), 6.87 (d, J=3.8 Hz, 1H), 6.80 (d, J=3.8 Hz, 1H), 6.59 (d, J=3.6 Hz, 1H), 5.85 (d, J=8.0 Hz, 1H), 4.82 (dd, J=5.2, 8.0 Hz, 1H), 4.19 (s, 1H), 4.14 (d, J=5.3 Hz, 1H), 1.54 (s, 3H).

### Biochemical Assay Protocol

Compounds were solubilized, and 3-fold diluted in 100% DMSO. These diluted compounds were further diluted in the assay buffer (50 mM Tris-HCl, pH 8.5, 50 mM NaCl, 5 mM MgCl₂, 0.01% Brij35, 1 mM DTT, 1% DMSO) for 10-dose IC₅₀ mode at a concentration 10-fold greater than the desired assay concentration. Standard reactions were performed in a total volume of 50 μl in assay buffer, with histone H2A (5 μM final) as substrate. To this was added the PRMT5/MEP50 complex diluted to provide a final assay concentration of 5 nM and the compounds were allowed to preincubate for 15 to 20 minutes at room temperature. The reaction was initiated by adding S-[3H-methyl]-adenosyl-L-methionine (PerkinElmer) to final concentration of 1 μM. Following a 60 minutes incubation at 30° C., the reaction was stopped by adding 100 μL of 20% TCA. Each reaction was spotted onto filter plate (MultiScreen FB Filter Plate, Millipore), and washed 5 times with PBS buffer, Scintillation fluid was added to the filter plate and read in a scintillation counter. IC₅₀ values were determined by fitting the data to the standard 4 parameters with Hill Slope using GraphPad Prism software.

### Cellular Assay Protocol

Cell Treatment and Western Blotting for Detecting Symmetric Di-Methyl Arginine (sDMA) and Histone H3R8 Dimethyl Symmetric (H3R8me2s) Marks

Initial Compounds Screening in A549 Cells:

Compounds were dissolved in DMSO to make 10 mM stock and further diluted to 0.1, and 1 mM. A549 cells were maintained in PRMI 1640 (Corning Cellgro, Catalog #: 10-040-CV) medium supplemented with 10% v/v FBS (GE Healthcare, Catalog #: SH30910.03). One day before experiment, 1.25×10⁵ cells were seeded in 6 well plate in 3 mL medium and incubated overnight. The next day, medium was changed and 3 uL of compound solution was added (1:1,000 dilution, 0.1 and 1 uM final concentration; DMSO concentration: 0.1%), and incubated for 3 days. Cells incubated with DMSO was used as a vehicle control. Cells were washed once with PBS, trypsinized in 150 uL 0.25% Trypsin (Corning, Catalog #: 25-053-CI), neutralized with 1 mL complete medium, transferred to microcentrifuge tubes and collected. Cell pellet was then resuspended in 15 uL PBS, lysed in 4% SDS, and homogenized by passing through homogenizer column (Omega Biotek, Catalog #: HCR003). Total protein concentrations were determined by BCA assay (ThermoFisher Scientific, Catalog #: 23225). Lysates were mixed with 5× Laemmli buffer and boiled for 5 min. Forty ug of total protein was separated on SDS-PAGE gels (Bio-Rad, catalog #: 4568083, 4568043), transferred to PVDF membrane, blocked with 5% dry milk (Bio-Rad, Catalog #: 1706404) in TBS with 0.1% v/v Tween 20 (TBST) for 1 hour at room temperature (RT), and incubated with primary antibodies (sDMA: Cell signaling, Catalog #: 13222, 1:3,000; H3R8me2s: Epigentek, Catalog #: A-3706-100, 1:2,000; β-Actin: Abcam, Catalog #: ab8227, 1:10,000) in 5% dry milk in TBST at 4° C. for overnight. The next day, membranes were washed with TBST, 5×5 min, and incubated with hP conjugated seconded antibody (GE Healthcare; Catalog #: NA934-1ML; 1:5,000) for 2 hours at RT, followed by 5×5 min washes with TBST, and incubation with ECL substrates (Bio-Rad, Catalog #: 1705061, 1705062). Chemiluminescent signal was captured with FluoChem HD2 imager (Proteinsimple) and analyzed by ImageJ.

To determine enzyme inhibition IC₅₀ values using Western Blot analysis, Granta cells were seeded at density of 5×10⁵ cells/mL in 3 mL medium (PRMI+10% v/v FBS). Nine-point 3-fold serial dilutions of compound were added to cells (3 ul, 1:1,000 dilution, DMSO concentration was 0.1%; final top concentration was 10 or 1 uM, depending on compounds potency) and incubated for 3 days. Cells incubated with DMSO was used as a vehicle control. Cells were harvested and subjected to western blot analysis as described above. SmD3me2s and H3R8me2s bands were quantified by ImageJ. Signals were normalized to β-Actin and DMSO control. IC₅₀ values were calculated using Graphpad Prism.

TABLE 7
Biochemical and cellular potency (in Granta-519 cell line)

| Ex. | PRMT5/MEP | PRMT5/MEP | sDMA IC₅₀ | |
|---|---|---|---|---|
| No | 50 IC₅₀ (μM) | 50_N | (μM) | sDMA_N |
| 20 | 0.0006 | 2 | 120 | 2 |
| 36 | 17.3 | 3 | 102 | 9 |
| 37 | 0.0024 | 1 | 0.008 | 3 |
| 52 | 0.188 | 1 | 39% | 1 |
| | | | inhibition @ | |
| | | | 1 uM | |
| 39 | 0.18 | 1 | | |
| 62 | 0.0086 | 2 | 0.045 | 4 |
| 63 | 0.0007 | 2 | 0.0108 | 2 |
| 64 | 0.0083 | 2 | 0.093 | 1 |
| 54 | 0.0256 | 1 | 55% | |
| | | | inhibition @ | |
| | | | 1 uM | |
| 53 | 0.526 | 1 | | |
| 65 | 0.0019 | 1 | 0.016 | 2 |
| 66 | 0.0004 | 1 | 0.0018 | 1 |
| 67 | 0.0006 | 1 | 0.01 | 1 |
| 68 | 0.035 | 1 | 0.09 | 2 |
| 69 | 0.0018 | 3 | 0.028 | 3 |
| 70 | 0.0102 | 1 | 0.048 | 2 |
| 59 | 0.0028 | 2 | 0.045 | 1 |
| 71 | 0.0139 | 1 | 0.26 | 1 |
| 72 | 0.0028 | 1 | 0.041 | 1 |
| 73 | 0.0097 | 1 | 0.313 | 1 |
| 74 | 0.0756 | 1 | | |
| 75 | 0.0954 | 1 | | |
| 76 | 0.0027 | 1 | | |
| 77 | 0.1176 | 1 | | |
| 78 | 0.0031 | 1 | 0.033 | 1 |
| 61 | 0.0026 | 2 | 0.033 | 2 |
| 79 | 6.46 | 1 | | |
| 60 | 0.0377 | 1 | 0.438 | 1 |
| 80 | 0.0007 | 1 | 0.018 | 1 |
| 81 | 0.0003 | 1 | 0.009 | 1 |
| 82 | 0.0039 | 1 | 0.011 | 1 |
| 83 | 0.0291 | 1 | 0.097 | 1 |
| 92 | 1.97 | 2 | | |
| 93 | 0.92 | 3 | | |
| 94 | 1.62 | 1 | | |
| 95 | 0.699 | 1 | | |
| 96 | | | 0.142 | 1 |
| 97 | 0.042 | 1 | | |
| 98 | 0.0084 | 1 | 0.009 | |
| 99 | 0.0024 | 1 | 0.0084 | 1 |
| 100 | 1.77 | 1 | | |
| 101 | 0.308 | 1 | | |
| 102 | 0.436 | 1 | 3.36 | 1 |
| 103 | 0.0296 | 1 | 0.0579 | 1 |
| 104 | 0.0102 | 1 | | |
| 105 | 0.0002 | 1 | | |
| 106 | 0.113 | 1 | | |
| 107 | 0.028 | 1 | | |
| 108 | 0.0096 | 1 | 0.131 | 1 |

TABLE 8
Biochemical and cellular potency (in Granta cell line)—Compounds
of Formula III and Formula IV

| Ex. | PRTMT5/ME | PRTMT5/MEP5 | sDMA IC₅₀ | |
|---|---|---|---|---|
| No | P50 IC₅₀ μM | 0_N | μM | sDMA_N |
| 25-B | 0.047 | 1 | ~3 | 1 |
| 28-B | 1.16 | 1 | | |
| 37-B | 0.0063 | 1 | 100 | 1 |
| 49-B | 0.543 | 1 | | |
| 50-B | 0.0117 | 1 | ~0.6 | 1 |
| 79-B | 0.0082 | 1 | 0.097 | 1 |
| 84-B | 0.0027 | 1 | 0.016 | 1 |
| 85-B | 0.00064 | 1 | 0.0009 | 1 |
| 86-B | 0.0002 | 1 | 0.003 | 1 |
| 87-B | 0.00033 | 1 | 0.0017 | 1 |
| 88-B | 0.037 | 1 | | |
| 89-B | 0.095 | 1 | | |
| 90-B | 0.00082 | 1 | 0.028 | 1 |
| 91-B | 0.0032 | 1 | 0.0468 | 1 |
| 92-B | 0.0199 | 1 | 0.335 | 1 |
| 93-B | 0.0023 | 1 | | |
| 94-B | 0.0034 | 1 | 0.053 | 1 |

TABLE 9
Biochemical and cellular potency (in Granta cell line)—Compounds
of Formula V and Formula VI

| Ex. | PRTMT5/ME | PRTMT5/MEP5 | sDMA IC₅₀ | |
|---|---|---|---|---|
| No | P50 IC₅₀ μM | 0_N | μM | sDMA_N |
| 1-C | 0.0036 | 2 | 0.0082 | 2 |
| 2-C | 0.0135 | 1 | 0.013 | 1 |
| 3-C | 0.0009 | 1 | 0.0038 | 1 |
| 5-C | 0.037 | 1 | 0.091 | 1 |
| 6-C | 0.0069 | 1 | 0.093 | 1 |
| 15-C | 0.001 | 1 | 0.0019 | 1 |
| 21-C | 0.0009 | 1 | 0.007 | 1 |

### FaSSIF Solubility

Compounds were first dispersed in freshly prepared FaSSIF (http://biorelevant.com/site_media/upload/documents/How_to_make_FaSSIF_FeSSIF_and_FaSSGF.pdf) buffer in 1 mg/mL respectively, and the standard samples were prepared by preparing 1 mg/mL of test compounds in DMSO. The compounds were then sufficient mixed by vortex mixer for 30 sec, and agitated at 25° C. using 300 rpm form 4 hour in thermo mixer. After incubation, the prepared samples were centrifuged at 10000 rpm for 10 min to remove the undissolved solid, the resulting supernatants were applied to HPLC. The actual concentrations of the compounds were evaluated by measuring the peak area, and the solubility (S) of compounds was calculated according to following equation:

S=Cₛₘₚ=C_{std}*(Aₛₘₚ/A_{std})*(V_{std}/Vₛₘₚ)

Where C is the sample concentration in μg/mL, A is the peak area, and V is the injection volume.

Warfarin (10-25 μg/mL), Atovaquone (<2 μg/mL) and Nimesulide (100-200 μg/mL) are positive controls in this experiment.

Example 36 was measured to have a FaSSIF solubility of 912.1 μg/mL.

Example 92A was measured to have an average of FaSSIF solubility of 54.7 μg/mL (n=4).

### In Vivo Pharmacokinetic Properties of Example 36.

In a rat (SD, male, non-fasted) non-crossover cassette PK study, Example 36 was dosed at 0.25 mg/kg via i.v. administration (N=3) and 2 mg/kg via oral gauge (p.o.) (N=3) with other 3 compounds. It showed average TI/2 of 1.2 h, Vss of 1.1 L/kg, blood clearance of 12.3 mL/min/kg in the i.v. group; it showed average dose normalized AUC of 1738 ng*h*kg/mL/mg and >120% of oral bioavailability in the p.o. group.

### In Vivo Pharmacokinetic Properties of Example 92.

In a rat (SD, male, non-fasted) non-crossover PK study, Example 92 was dosed at 1 mg/kg (DMA: 20% HPBCD=5:95, solution) via i.v. administration (N=2) and 1 mg/kg (0.5% Na CMC+0.5% Tween80, solution) via oral gauge (p.o.) (N=2). It showed average T^{1/2} of 1.84 hr, Vss of 1.13 L/kg, blood clearance of 11.3 mL/min/kg in the i.v. group; it showed average dose normalized AUC_{0-inf} of 2184 ng*h*kg/mL/mg and >100% of oral bioavailability in the p.o. group; The metabolite (Ex. 1-C) was detected in blood samples. It showed T_{1/2} of 5.01 hr, Tₘₐₓ of 8.00 hr, average of AUC₀₋ᵢₙₜ of 284 ng*h*kg/mL/mg.

### In Vivo Efficacy of Example 92 in a Rat Type II Collagen-Induced Arthritis (CIA) Model

Female Lewis rats were injected intradermally/subcutaneously (ID/SC) with porcine type II collagen to induce arthritis on Study Days 0 & 7. Rats were then dosed once daily (QD) on Study Days 0-24 (developing) or Days 6-24 (semi-established) by the oral (PO) route with Example 92 (15 mg/kg) or vehicle (0.5% Na CMC+0.5% Tween80, suspension). Rats were euthanized for necropsy on Study Day 25. Efficacy evaluation was based daily ankle diameter caliper measurements, terminal hind paw weights, and histopathology of ankles and knees.

Administration of Example 92 at 15 mg/kg resulted in a 63% reduction in ankle diameter (P<0.001) and 67% reduction in hind paw weight (P<0.001) in developing mode as well as 34% reduction in ankle diameter (P<0.05) and 51% reduction in hind paw weight (P<0.01) in semi-established mode. Treatment in both modes also significantly inhibited all histopathology parameters in ankles and knees as compared to vehicle controls

### In Vivo Efficacy of Example 92 in a MOG₃₅₋₅₅/CFA Induced Mouse Model of Experimental Autoimmune Encephalomyelitis

Female C57BL/6 mice were injected subcutaneously at two sites in the back with the emulsion component (containing MOG₃₅₋₅₅) of Hooke Kit™ MOG₃₅₋₅₅/CFA Emulsion PTX, catalog number EK-2110 (Hooke Laboratories, Lawrence Mass.). Animals were then dosed orally once daily with Example 92 at 30 mg/kg on study days 0-8 and at 20 mg/kg on study days 13-28. Efficacy was assessed based on body weight change and EAE score.

Once daily oral administration of Example 92 was efficacious at postponing disease onset (Mean day of disease onset-24.6 versus 13.8 for vehicle, P<0.001) and reducing disease severity (Maximum score−1.38 versus 3.25 for vehicle, P<0.001) in this model.

1. A method of treating a disease or disorder that is rejection of transplanted organs or tissue; graft-versus-host diseases brought about by transplantation; autoimmune syndromes, multiple sclerosis, myasthenia gravis; pollen allergies; type I diabetes; prevention of psoriasis; Crohn's disease; ulcerative colitis, acute respiratory distress syndrome; adult respiratory distress syndrome; influenza; COVID-19 (coronavirus disease); or post-infectious autoimmune diseases including rheumatic fever and post-infectious glomerulonephritis; in a patient in need thereof, comprising administering to said patient an effective amount of a compound of Formula I or Formula II: 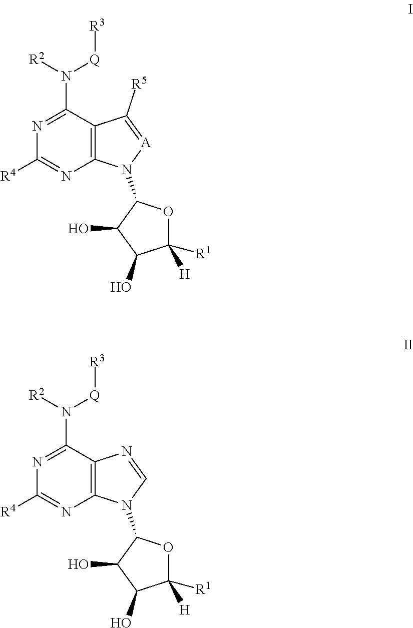
or a pharmaceutically acceptable salt or solvate thereof;
wherein
A is CH or N;
Q is NH, NR⁶ or O;
R¹ is —C₀-C₆alk-C₃-C₆cycloalkyl, —C₀-C₆alk-C₃-C₆halocycloalkyl; —C₂-C₆alkenyl, —C₂-C₆haloalkenyl, —C₀-C₆alk-C₁-C₆alkyl, —C₀-C₆alk-C₁-C₆haloalkyl, —C₀-C₆alk-C≡CH, —C₀-C₆alk-C≡C—C₁-C₆alkyl, —C₀-C₆alk-C≡C—C₁-C₆haloalkyl, —C₀-C₆alk-C≡C—C₃-C₆cycloalkyl, —C₁-C₆alk-aryl, —C₁-C₆alk-S—C₁-C₆alkyl, —C₁-C₆alk-S—C₁-C₆haloalkyl, —C₁-C₆alk-S—C₃-C₆cycloalkyl; —C₁-C₆alk-S—C₃-C₆halocycloalkyl; —C₁-C₆alk-O—C₁-C₆alkyl, —C₁-C₆alk-O—C₃-C₆cycloalkyl, —C₁-C₆alk-S—CH₂-aryl, —C₁-C₆alk-C(O)NH-aryl, —C₀-C₆alk-heteroaryl, —C₁-C₆alk-O-heteroaryl, —C₁-C₆alk-S-heteroaryl, or —C₁-C₆alk-NH-heteroaryl;
R² is H, —C₁-C₆alkyl, —C₁-C₆haloalkyl, or —C₀-C₆alk-C₃-C₆cycloalkyl;
R³ is H, —C₁-C₆alkyl, —C₁-C₆haloalkyl, —C₀-C₆alk-C₃-C₆cycloalkyl, —C(O)R⁷, —C(O)OR⁷, or —C(O)NR^{8a}R^{8b};
R⁴ is H, halo, —C₁-C₆alkyl, or NH₂;
R⁵ is H, halo, CN, —C₁-C₆alkyl, —C₂-C₄alkenyl, —C₂-C₄haloalkenyl, C₂-C₄cyanoalkenyl, —C₀-C₆alk-C≡CH, —C₀-C₆alk-C≡C—C₁-C₆alkyl, —C₁-C₄haloalkyl, —C₂-C₆heterocycloalkyl, oxo-substituted-C₂-C₆heterocycloalkyl, —C₃-C₆cycloalkyl, —C₀-C₃alk-C(O)R⁹, —CR⁸R^{8′}CN, —CH₂NR⁸R^{8′}, —C₀-C₆alk-OH, —NR⁸R^{8′}, —N(R⁹)CN, —O—C₁-C₄alkyl, —NR⁹CONR⁸R^{8′}, —OCONR⁸R^{8′}, or —NR⁹C(O)OR^{9a};
R⁶ is —C₁-C₆alkyl or —C₀-C₆alk-C₃-C₆cycloalkyl;
R⁷ is H, C₁-C₆alkyl, or C₀-C₆alk-C₃-C₆cycloalkyl;
R^{8a} and R^{8b} are each independently H, C₁-C₆alkyl, or —C₀-C₆alk-OC₁-C₆alkyl, or R^{8a} and R^{8b}, together with the atom to which they are attached, form a C₂-C₆heterocycloalkyl ring;
R⁸ and R^{8′} are each independently H, C₁-C₆alkyl, or —C₀-C₆alk-OC₁-C₆alkyl;
or R⁸ and R^{8′}, together with the atom to which they are attached, form a C₃-C₆cycloalkyl ring or a C₂-C₆heterocycloalkyl ring;
R⁹ is H, —C₁-C₆alkyl, or —C₀-C₆alk-C₃-C₆cycloalkyl; and
R^{9a} is —C₁-C₆alkyl, or C₀-C₆alk-C₃-C₆cycloalkyl.

2. The method of claim 1 wherein R¹ is —C₀-C₆alk-C₁-C₆alkyl, preferably —CH(OH)—C₁-C₆alkyl, —CH(F)—C₁-C₆alkyl, —CH(NH₂)—C₁-C₆alkyl, —CH(Me)-C₁-C₆alkyl, or —C(Me)(OH)—C₁-C₆alkyl.

3. The method of claim 1 wherein R¹ is —C₀-C₆alk-C₁-C₆haloalkyl, preferably —CH(OH)—C₁-C₆haloalkyl, —CH(F)—C₁-C₆haloalkyl, —CH(NH₂)—C₁-C₆haloalkyl, —CH(Me)-C₁-C₆haloalkyl, or —C(Me)(OH)—C₁-C₆haloalkyl.

4. The method of claim 1 wherein R¹ is —C₀-C₆alk-C≡CH, preferably —CH(OH)—C≡CH, —CH(F)—C≡CH, —CH(NH₂)—C≡CH, —CH(Me)-C≡CH, or —C(Me)(OH)—C≡CH.

5. The method of claim 1 wherein R¹ is —C₀-C₆alk-C≡C—C₁-C₆alkyl, preferably —CH(OH)—C≡C—C₁-C₆alkyl, —CH(F)—C≡C—C₁-C₆alkyl, —CH(NH₂)—C—C≡C₁-C₆alkyl, —CH(Me)-C≡C—C₁-C₆alkyl, or —C(Me)(OH)—C≡C—C₁-C₆alkyl, more preferably —CH(OH)—C≡C—CH₃, —CH(F)—C≡C—CH₃, —CH(NH₂)—C≡C—CH₃, —CH(Me)-C≡C—CH₃, or —C(Me)(OH)—C≡C—CH₃.

6. The method of claim 1 wherein R¹ is —C₀-C₆alk-C≡C—C₁-C₆haloalkyl, preferably —CH(OH)—C≡C—C₁-C₆haloalkyl, —CH(F)—C≡C—C₁-C₆haloalkyl, —CH(NH₂)—C≡C—C₁-C₆haloalkyl, —CH(Me)-C≡C—C₁-C₆haloalkyl, or —C(Me)(OH)—C≡C—C₁-C₆haloalkyl, more preferably —CH(OH)—C≡C—CF₃, —CH(F)—C≡C—CF₃, —CH(NH₂)—C≡C—CF₃, —CH(Me)-C≡C—CF₃, or —C(Me)(OH)—C≡C—CF₃.

7. The method of claim 1 wherein R¹ is —C₀-C₆alk-C≡C—C₃-C₆cycloalkyl, preferably —CH(OH)—C≡C—C₃-C₆cycloalkyl, —CH(F)—C≡C—C₃-C₆cycloalkyl, —CH(NH₂)—C≡C—C₃-C₆cycloalkyl, —CH(Me)-C≡C—C₃-C₆cycloalkyl, or —C(Me)(OH)—C≡C—C₃-C₆cycloalkyl, more preferably —CH(OH)—C≡C-cyclopropyl, —CH(F)—C≡C-cyclopropyl, —CH(NH₂)—C≡C-cyclopropyl, —CH(Me)-C≡C-cyclopropyl, or —C(Me)(OH)—C≡C-cyclopropyl.

8. The method of claim 1 wherein R¹ is —C₁-C₆alk-aryl, preferably —CH(OH)-aryl, —C(OCH₃)-aryl, —CH(F)-aryl, —CH(NH₂)-aryl, —CH(Me)-aryl, or —C(Me)(OH)-aryl, more preferably —CH(OH)-4-chlorophenyl, —CH(OH)-3,4-dichlorophenyl, —CH(OH)-3,4-difluorophenyl, —CH(OH)-3-fluoro-4-chlorophenyl, —CH(OH)-3-chloro-4-fluorophenyl, —CH(OH)-4-(trifluoromethyl)phenyl, —CH(OH)-3-fluoro-4-(trifluoromethyl)phenyl, —CH(OH)-3-methyl-4-(trifluoromethyl)phenyl, —C(CF₃)(OH)-4-chlorophenyl, —CH(OH)-3-methyl-4-chlorophenyl, —CH(OH)-2,3-dihydrobenzofuran-5-yl, —CH(OH)-benzo[d][1,3]dioxol-5-yl, —CH(F)-4-chlorophenyl, —CH(F)-3,4-dichlorophenyl, —CH(F)-3,4-difluorophenyl, —CH(F)-3-fluoro-4-chlorophenyl, —CH(F)-3-chloro-4-fluorophenyl, —CH(F)-4-(trifluoromethyl)phenyl, —CH(F)-3-fluoro-4-(trifluoromethyl)phenyl, —CH(F)-3-methyl-4-(trifluoromethyl)phenyl, —C(CF₃)(F)-4-chlorophenylphenyl, —CH(F)-3-methyl-4-chlorophenyl, —CH(F)-2,3-dihydrobenzofuran-5-yl, —CH(F)-benzo[d][1,3]dioxol-5-yl, —CH(NH₂)-4-chlorophenyl, —CH(NH₂)-3,4-dichlorophenyl, —CH(NH₂)-3,4-difluorophenyl, —CH(NH₂)-3-fluoro-4-chlorophenyl, —CH(NH₂)-3-chloro-4-fluorophenyl, —CH(NH₂)-4-(trifluoromethyl)phenyl, —CH(NH₂)-3-fluoro-4-(trifluoromethyl)phenyl, —CH(NH₂)-3-methyl-4-(trifluoromethyl)phenyl, —C(CF₃)(NH₂)-4-chlorophenylphenyl, CH(NH₂)-3-methyl-4-chlorophenyl, —CH(NH₂)-2,3-dihydrobenzofuran-5-yl, —CH(NH₂)-benzo[d][1,3]dioxol-5-yl, —CH(Me)-4-chlorophenyl, —CH(Me)-3,4-dichlorophenyl, —CH(Me)-3,4-difluorophenyl, —CH(Me)-3-fluoro-4-chlorophenyl, —CH(Me)-3-chloro-4-fluorophenyl, —CH(Me)-4-(trifluoromethyl)phenyl, —CH(Me)-3-methyl-4-(trifluoromethyl)phenyl, —CH(Me)-3-fluoro-4-(trifluoromethyl)phenyl, —CH(Me)-3-methyl-4-chlorophenyl, —C(CF₃)(Me)-4-chlorophenylphenyl, —CH(Me)-2,3-dihydrobenzofuran-5-yl, —CH(Me)-benzo[d][1,3]dioxol-5-yl, —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, —C(Me)(OH)-4-(trifluoromethyl)phenyl, —C(Me)(OH)-3-fluoro-4-(trifluoromethyl)phenyl, —C(Me)(OH)-3-methyl-4-(trifluoromethyl)phenyl, —C(Me)(OH)-3-methyl-4-chlorophenyl, —C(Me)(OH)-2,3-dihydrobenzofuran-5-yl, or —C(Me)(OH)-benzo[d][1,3]dioxol-5-yl.

9. The method of claim 1, wherein R¹ is —C₀-C₆alk-heteroaryl, —C₁-C₆alk-O-heteroaryl,
—C₁-C₆alk-S-heteroaryl, or —C₁-C₆alk-NH-heteroaryl, preferably wherein the —C₀-C₆alk-heteroaryl is 2-(2-amino-3-bromoquinolin-7-yl)ethyl or 2-(2-amino-3-chloroquinolin-7-yl)ethyl.

10.-24. (canceled)

25. A method of treating a disease or disorder that is rejection of transplanted organs or tissue; graft-versus-host diseases brought about by transplantation; autoimmune syndromes, multiple sclerosis, myasthenia gravis; pollen allergies; type I diabetes; prevention of psoriasis; Crohn's disease; ulcerative colitis, acute respiratory distress syndrome; adult respiratory distress syndrome; influenza; COVID-19 (coronavirus disease); or post-infectious autoimmune diseases including rheumatic fever and post-infectious glomerulonephritis in a patient in need thereof, comprising administering to said patient an effective amount of a compound of Formula III or Formula IV: 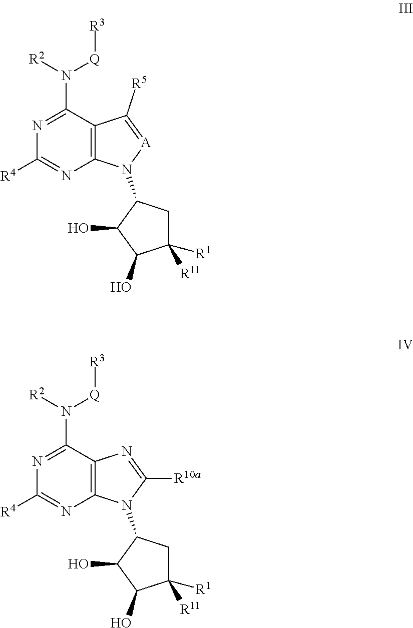
or a pharmaceutically acceptable salt or solvate thereof;
wherein
A is CH, CR¹⁰, or N;
Q is NH, NR⁶, or O;
R¹ is —C₀-C₆alk-C₃-C₆cycloalkyl, —C₀-C₆alk-C₃-C₆halocycloalkyl, —C₂-C₆alkenyl, —C₂-C₆haloalkenyl, —C₀-C₆alk-C₁-C₆alkyl, —C₀-C₆alk-C₁-C₆haloalkyl, —C₀-C₆alk-C≡CH, —C₀-C₆alk-C≡C—C₁-C₆alkyl, —C₀-C₆alk-C≡C—C₁-C₆haloalkyl, —C₀-C₆alk-C≡C—C₃-C₆cycloalkyl, —C₁-C₆alk-aryl, —C₁-C₆alk-S—C₁-C₆alkyl, —C₁-C₆alk-S—C₁-C₆haloalkyl, —C₁-C₆alk-S—C₃-C₆cycloalkyl; —C₁-C₆alk-S—C₃-C₆halocycloalkyl; —C₁-C₆alk-O—C₁-C₆alkyl, —C₁-C₆alk-O—C₃-C₆cycloalkyl, —C₁-C₆alk-S—CH₂-aryl, —C₁-C₆alk-C(O)NH-aryl, —C₀-C₆alk-S-aryl, —C₀-C₆alk-S(O)aryl, —C₀-C₆alk-S(O)₂aryl, —C₀-C₆alk-Oaryl, —C₀-C₆alk-heteroaryl, —C₁-C₆alk-O-heteroaryl, —C₁-C₆alk-S-heteroaryl, or —C₁-C₆alk-NH-heteroaryl;
R² is H, —C₁-C₆alkyl, —C₁-C₆haloalkyl, or —C₀-C₆alk-C₃-C₆cycloalkyl;
R³ is H, —C₁-C₆alkyl, —C₁-C₆haloalkyl, —C₀-C₆alk-C₃-C₆cycloalkyl, —C(O)R⁷, —C(O)OR⁷, or —C(O)NR^{8a}R^{8b};
R⁴ is H, halo, —C₁-C₆alkyl, or NH₂;
R⁵ is H, halo, CN, —C₁-C₆alkyl, —C₂-C₄alkenyl, —C₂-C₄haloalkenyl, C₂-C₄cyanoalkenyl, —C₀-C₆alk-C≡CH, —C₀-C₆alk-C≡C—C₁-C₆alkyl, —C₁-C₄haloalkyl, —C₂-C₆heterocycloalkyl, oxo-substituted-C₂-C₆heterocycloalkyl, —C₃-C₆cycloalkyl, —C₀-C₃alk-C(O)R⁹, —CR⁸R^{8′}CN, —CH₂NR⁸R^{8′}, —C₀-C₆alk-OH, —NR⁸R^{8′}, —N(R⁹)CN, —O—C₁-C₄alkyl, —NR⁹CONR⁸R^{8′}, —OCONR⁸R^{8′}, or —NR⁹C(O)OR^{9a};
R⁶ is C₁-C₆alkyl, or C₀-C₆alk-C₃-C₆cycloalkyl
R⁷ is H, C₁-C₆alkyl, or C₀-C₆alk-C₃-C₆cycloalkyl;
R^{8a} and R^{8b} are each independently H, C₁-C₆alkyl, or —C₀-C₆alk-OC₁-C₆alkyl, or R^{8a} and R^{8b}, together with the atom to which they are attached, form a C₂-C₆heterocycloalkyl ring;
R¹ and R^{8′} are each independently H, C₁-C₆alkyl, or —C₀-C₆alk-OC₁-C₆alkyl;
or R⁸ and R^{8′}, together with the atom to which they are attached, form a C₃-C₆cycloalkyl ring or a C₂-C₆heterocycloalky ring;
R⁹ is H, —C₁-C₆alkyl, or C₀-C₆alk-C₃-C₆cycloalkyl;
R^{9a} is —C₁-C₆alkyl, or C₀-C₆alk-C₃-C₆cycloalkyl;
R¹⁰ is halo or —C₁-C₆alkyl;
R^{10a} is H, halo, or —C₁-C₆alkyl; and
R¹¹ is H, —C₁-C₆alkyl, —C₁-C₆haloalkyl, —C₀-C₆alk-C₃-C₆cycloalkyl, —C₀-C₆alk-C₃-C₆halocycloalkyl, —C₀-C₆alk-OH, —C₀-C₆alk-NH₂, —C₀-C₆alk-NH—C₁-C₆alkyl, —C₀-C₆alk-N(C₁-C₆alkyl)-C₁-C₆alkyl, —C₀-C₆alk-NH—C₃-C₆cycloalkyl, or —C₀-C₆alk-N(C₁-C₆alkyl)-C₃-C₆cycloalkyl;
or R¹¹ and R¹, together with the atom to which they are attached, form a C₃-C₆cycloalkyl ring or a heterocycloalkyl ring.

26. The method of claim 25, wherein R₁ is —C₀-C₆alk-C₁-C₆alkyl, preferably —CH(OH)—C₁-C₆alkyl, —CH(F)—C₁-C₆alkyl, —CH(NH₂)—C₁-C₆alkyl, —CH(Me)-C₁-C₆alkyl, or —C(Me)(OH)—C₁-C₆alkyl.

27. The method of claim 25, wherein R₁ is —C₀-C₆alk-C₁-C₆haloalkyl, preferably —CH(OH)—C₁-C₆haloalkyl, —CH(F)—C₁-C₆haloalkyl, —CH(NH₂)—C₁-C₆haloalkyl, —CH(Me)-C₁-C₆haloalkyl, or —C(Me)(OH)—C₁-C₆haloalkyl.

28. The method of claim 25, wherein R₁ is —C₀-C₆alk-C≡CH, preferably —CH(OH)—C≡CH, —CH(F)—C≡CH, —CH(NH₂)—C≡CH, —CH(Me)-C≡CH, or —C(Me)(OH)—C≡CH.

29. The method of claim 25, wherein R₁ is —C₀-C₆alk-C≡C—C₁-C₆alkyl, preferably —CH(OH)—C≡C—C₁-C₆alkyl, —CH(F)—C≡C—C₁-C₆alkyl, —CH(NH₂)—C≡C—C₁-C₆alkyl, —CH(Me)-C≡C—C₁-C₆alkyl, or —C(Me)(OH)—C≡C—C₁-C₆alkyl, more preferably —CH(OH)—C≡C—CH₃, —CH(F)—C≡C—CH₃, —CH(NH₂)—C≡C—CH₃, —CH(Me)-C≡C—CH₃, or —C(Me)(OH)—C≡C—CH₃.

30. The method of claim 25, wherein R₁ is —C₀-C₆alk-C≡C—C₁-C₆haloalkyl, preferably —CH(OH)—C≡C—C₁-C₆haloalkyl, —CH(F)—C≡C—C₁-C₆haloalkyl, —CH(NH₂)—C≡C—C₁-C₆haloalkyl, —CH(Me)-C≡C—C₁-C₆haloalkyl, or —C(Me)(OH)—C≡C—C₁-C₆haloalkyl, more preferably —CH(OH)—C≡C—CF₃, —CH(F)—C≡C—CF₃, —CH(NH₂)—C≡C—CF₃, —CH(Me)-C≡C—CF₃, or —C(Me)(OH)—C≡C—CF₃.

31. The method of claim 25, wherein R₁ is —C₀-C₆alk-C≡C—C₃-C₆cycloalkyl, preferably —CH(OH)—C≡C—C₃-C₆cycloalkyl, —CH(F)—C≡C—C₃-C₆cycloalkyl, —CH(NH₂)—C≡C—C₃-C₆cycloalkyl, —CH(Me)-C≡C—C₃-C₆cycloalkyl, or —C(Me)(OH)—C≡C—C₃-C₆cycloalkyl, more preferably —CH(OH)—C≡C-cyclopropyl, —CH(F)—C≡C-cyclopropyl, —CH(NH₂)—C≡C-cyclopropyl, —CH(Me)-C≡C-cyclopropyl, or —C(Me)(OH)—C≡C-cyclopropyl.

32. The method of claim 25, wherein R₁ is —C₁-C₆alk-aryl, preferably —CH₂-aryl, —CH(OH)-aryl, —CH(F)-aryl, —CH(NH₂)-aryl, —CH(Me)-aryl, or —C(Me)(OH)-aryl, more preferably —CH₂-4-chlorophenyl, —CH₂-3,4-dichlorophenyl, —CH₂-3,4-difluorophenyl, —CH₂-3-fluoro-4-chlorophenyl, —CH₂-3-chloro-4-fluorophenyl, —CH(OH)-4-chlorophenyl, —CH(OH)-3,4-dichlorophenyl, —CH(OH)-3,4-difluorophenyl, —CH(OH)-3-fluoro-4-chlorophenyl, —CH(OH)-3-chloro-4-fluorophenyl, —CH(F)-4-chlorophenyl, —CH(F)-3,4-dichlorophenyl, —CH(F)-3,4-difluorophenyl, —CH(F)-3-fluoro-4-chlorophenyl, —CH(F)-3-chloro-4-fluorophenyl, —CH(NH₂)-4-chlorophenyl, —CH(NH₂)-3,4-dichlorophenyl, —CH(NH₂)-3,4-difluorophenyl, —CH(NH₂)-3-fluoro-4-chlorophenyl, —CH(NH₂)-3-chloro-4-fluorophenyl, —CH(Me)-4-chlorophenyl, —CH(Me)-3,4-dichlorophenyl, —CH(Me)-3,4-difluorophenyl, —CH(Me)-3-fluoro-4-chlorophenyl, —CH(Me)-3-chloro-4-fluorophenyl, —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, or —C(Me)(OH)-3-chloro-4-fluorophenyl.

33. The method of claim 25, wherein R¹ is —C₀-C₆alk-S-aryl, preferably —S-4-chlorophenyl, —S-3,4-dichlorophenyl, —S-3,4-difluorophenyl, —S-3-fluoro-4-chlorophenyl, or —S-3-chloro-4-fluorophenyl.

34. The method of claim 25, wherein R¹ is —C₀-C₆alk-S(O)-aryl, preferably —S(O)-4-chlorophenyl, —S(O)-3,4-dichlorophenyl, —S(O)-3,4-difluorophenyl, —S(O)-3-fluoro-4-chlorophenyl, or —S(O)-3-chloro-4-fluorophenyl.

35. The method of claim 25, wherein R¹ is —C₀-C₆alk-S(O)₂-aryl, preferably —S(O)₂-4-chlorophenyl, —S(O)₂-3,4-dichlorophenyl, —S(O)₂-3,4-difluorophenyl, —S(O)₂-3-fluoro-4-chlorophenyl, or —S(O)₂-3-chloro-4-fluorophenyl.

36. The method of claim 25, wherein R¹ is —C₀-C₆alk-O-aryl, preferably —O-4-chlorophenyl, —O-3,4-dichlorophenyl, —O-3,4-difluorophenyl, —O-3-fluoro-4-chlorophenyl, or —O-3-chloro-4-fluorophenyl.

37. The method of claim 25, wherein R¹ is —C₀-C₆alk-heteroaryl, preferably 2-(2-amino-3-bromoquinolin-7-yl)ethyl, 2-(2-amino-3-chloroquinolin-7-yl)ethyl, 2-(2-((cyclopropylmethyl)amino)quinolin-7-yl)ethyl, 2-(2-(methylamino)quinolin-7-yl)ethyl, or 2-(2-aminoquinolin-7-yl)ethyl.

38. The method of claim 25, wherein R¹ is —C₁-C₆alk-O-heteroaryl, preferably ((2-amino-3-bromoquinolin-7-yl)oxy)methyl.

39. The method of claim 25, wherein R¹ is —C₁-C₆alk-S-heteroaryl, preferably ((2-amino-3-bromoquinolin-7-yl)thio)methyl.

40. The method of claim 25, wherein R¹ is —C₁-C₆alk-NH-heteroaryl, preferably 2-amino-3-bromoquinolin-7-yl)amino)methyl.

41.-59. (canceled)

60. A method of treating a disease or disorder that is rejection of transplanted organs or tissue; graft-versus-host diseases brought about by transplantation; autoimmune syndromes, multiple sclerosis, myasthenia gravis; pollen allergies; type I diabetes; prevention of psoriasis; Crohn's disease; ulcerative colitis, acute respiratory distress syndrome; adult respiratory distress syndrome; influenza; COVID-19 (coronavirus disease); or post-infectious autoimmune diseases including rheumatic fever and post-infectious glomerulonephritis in a patient in need thereof, comprising administering to said patient an effective amount of a compound of Formula V or Formula VI: 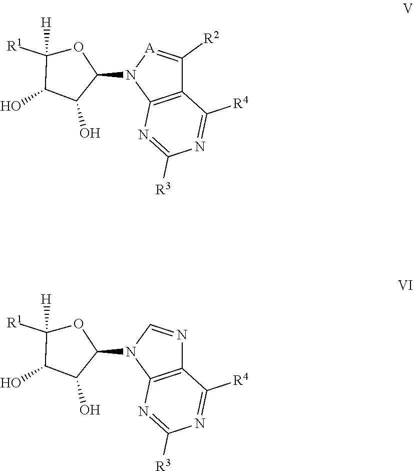
or a pharmaceutically acceptable salt or solvate thereof;
wherein
A is CH or N;
R¹ is —C₁-C₆alk-aryl, —C₁-C₆alk-heteroaryl, —C₁-C₆alk-C≡CH, —C₁-C₆alk-C≡C—C₁-C₆alkyl, —C₁-C₆alk-C≡C—C₁-C₆haloalkyl, or —C₁-C₆alk-C≡C—C₃-C₆cycloalkyl;
R² is H, or halo;
R³ is H, halo, NH₂, or C₁-C₆alkyl; and
R⁴ is NH₂ or CH₃.

61. The method of claim 60, wherein R¹ is —CH(OH)-aryl, —CH(Me)-aryl, —C(Me)(OH)-aryl, —CH(CH₂OH)-aryl, —C(Me)(OH)-heteroaryl, or —CH(OH)—C≡C—C₃-C₆cycloalkyl.

62. The method of claim 60, wherein R¹ is —C(Me)(OH)-4-chlorophenyl, —C(Me)(OH)-3,4-dichlorophenyl, —C(Me)(OH)-3,4-difluorophenyl, —C(Me)(OH)-3-fluoro-4-chlorophenyl, —C(Me)(OH)-3-chloro-4-fluorophenyl, —CH(Me)(OH)-3-methyl-4-chlorophenyl, —CH(Me)(OH)-3-fluoro-4-trifluoromethylphenyl, —CH(Me)(OH)-4-trifluoromethylphenyl, —CH(Me)(OH)-3-methyl-4-trifluoromethylphenyl, —CH(Me)(OH)-3-chloro-4-fluorophenyl, —CH(Me)-4-chlorophenyl, —CH(CH₂OH)-4-chlorophenyl, —C(Me)(OH)-5-chlorothiophen-2-yl, —CH(OH)—C≡C-cyclopropyl.

63.-69. (canceled)
